SEQUENCE LISTING (2 sequences)

SEQ ID NO: 1 - protein, 2414 aa, Homo sapiens
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSLGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 2 - DNA, 8779 bp, Homo sapiens
  source 1..8779: mol_type = unassigned DNA
gagaaggaggaggacagcgccgaggaggaagaggttgatggcggcggcggagctccgagagacctcggctgggcaggggccggccgtggcgggccggggactgcgcctctagagccgcgagttctcgggaattcgccgcagcggacgcgctcggcgaatttgtgctcttgtgccctcctccgggcttgggcccaggcccggcccctcgcacttgcccttaccttttctatcgagtccgcatccctctccagccactgcgacccggcgaagagaaaaaggaacttcccccaccccctcgggtgccgtcggagccccccagcccacccctgggtgcggcgcggggaccccgggccgaagaagagatttcctgaggattctggttttcctcgcttgtatctccgaaagaattaaaaatggccgagaatgtggtggaaccggggccgccttcagccaagcggcctaaactctcatctccggccctctcggcgtccgccagcgatggcacagattttggctctctatttgacttggagcacgacttaccagatgaattaatcaactctacagaattgggactaaccaatggtggtgatattaatcagcttcagacaagtcttggcatggtacaagatgcagcttctaaacataaacagctgtcagaattgctgcgatctggtagttcccctaacctcaatatgggagttggtggcccaggtcaagtcatggccagccaggcccaacagagcagtcctggattaggtttgataaatagcatggtcaaaagcccaatgacacaggcaggcttgacttctcccaacatggggatgggcactagtggaccaaatcagggtcctacgcagtcaacaggtatgatgaacagtccagtaaatcagcctgccatgggaatgaacacagggatgaatgcgggcatgaatcctggaatgttggctgcaggcaatggacaagggataatgcctaatcaagtcatgaacggttcaattggagcaggccgagggcgacagaatatgcagtacccaaacccaggcatgggaagtgctggcaacttactgactgagcctcttcagcagggctctccccagatgggaggacaaacaggattgagaggcccccagcctcttaagatgggaatgatgaacaaccccaatccttatggttcaccatatactcagaatcctggacagcagattggagccagtggccttggtctccagattcagacaaaaactgtactatcaaataacttatctccatttgctatggacaaaaaggcagttcctggtggaggaatgcccaacatgggtcaacagccagccccgcaggtccagcagccaggcctggtgactccagttgcccaagggatgggttctggagcacatacagctgatccagagaagcgcaagctcatccagcagcagcttgttctccttttgcatgctcacaagtgccagcgccgggaacaggccaatggggaagtgaggcagtgcaaccttccccactgtcgcacaatgaagaatgtcctaaaccacatgacacactgccagtcaggcaagtcttgccaagtggcacactgtgcatcttctcgacaaatcatttcacactggaagaattgtacaagacatgattgtcctgtgtgtctccccctcaaaaatgctggtgataagagaaatcaacagccaattttgactggagcacccgttggacttggaaatcctagctctctaggggtgggtcaacagtctgcccccaacctaagcactgttagtcagattgatcccagctccatagaaagagcctatgcagctcttggactaccctatcaagtaaatcagatgccgacacaaccccaggtgcaagcaaagaaccagcagaatcagcagcctgggcagtctccccaaggcatgcggcccatgagcaacatgagtgctagtcctatgggagtaaatggaggtgtaggagttcaaacgccgagtcttctttctgactcaatgttgcattcagccataaattctcaaaacccaatgatgagtgaaaatgccagtgtgccctccctgggtcctatgccaacagcagctcaaccatccactactggaattcggaaacagtggcacgaagatattactcaggatcttcgaaatcatcttgttcacaaactcgtccaagccatatttcctacgccggatcctgctgctttaaaagacagacggatggaaaacctagttgcatatgctcggaaagttgaaggggacatgtatgaatctgcaaacaatcgagcggaatactaccaccttctagctgagaaaatctataagatccagaaagaactagaagaaaaacgaaggaccagactacagaagcagaacatgctaccaaatgctgcaggcatggttccagtttccatgaatccagggcctaacatgggacagccgcaaccaggaatgacttctaatggccctctacctgacccaagtatgatccgtggcagtgtgccaaaccagatgatgcctcgaataactccacaatctggtttgaatcaatttggccagatgagcatggcccagccccctattgtaccccggcaaacccctcctcttcagcaccatggacagttggctcaacctggagctctcaacccgcctatgggctatgggcctcgtatgcaacagccttccaaccagggccagttccttcctcagactcagttcccatcacagggaatgaatgtaacaaatatccctttggctccgtccagcggtcaagctccagtgtctcaagcacaaatgtctagttcttcctgcccggtgaactctcctataatgcctccagggtctcaggggagccacattcactgtccccagcttcctcaaccagctcttcatcagaattcaccctcgcctgtacctagtcgtacccccacccctcaccatactcccccaagcataggggctcagcagccaccagcaacaacaattccagcccctgttcctacacctcctgccatgccacctgggccacagtcccaggctctacatccccctccaaggcagacacctacaccaccaacaacacaacttccccaacaagtgcagccttcacttcctgctgcaccttctgctgaccagccccagcagcagcctcgctcacagcagagcacagcagcgtctgttcctaccccaacagcaccgctgcttcctccgcagcctgcaactccactttcccagccagctgtaagcattgaaggacaggtatcaaatcctccatctactagtagcacagaagtgaattctcaggccattgctgagaagcagccttcccaggaagtgaagatggaggccaaaatggaagtggatcaaccagaaccagcagatactcagccggaggatatttcagagtctaaagtggaagactgtaaaatggaatctaccgaaacagaagagagaagcactgagttaaaaactgaaataaaagaggaggaagaccagccaagtacttcagctacccagtcatctccggctccaggacagtcaaagaaaaagattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggaccgctttgtctacacctgcaatgaatgcaagcaccatgtggagacacgctggcactgtactgtctgtgaggattatgacttgtgtatcacctgctataacactaaaaaccatgaccacaaaatggagaaactaggccttggcttagatgatgagagcaacaaccagcaggctgcagccacccagagcccaggcgattctcgccgcctgagtatccagcgctgcatccagtctctggtccatgcttgccagtgtcggaatgccaattgctcactgccatcctgccagaagatgaagcgggttgtgcagcataccaagggttgcaaacggaaaaccaatggcgggtgccccatctgcaagcagctcattgccctctgctgctaccatgccaagcactgccaggagaacaaatgcccggtgccgttctgcctaaacatcaagcagaagctccggcagcaacagctgcagcaccgactacagcaggcccaaatgcttcgcaggaggatggccagcatgcagcggactggtgtggttgggcagcaacagggcctcccttcccccactcctgccactccaacgacaccaactggccaacagccaaccaccccgcagacgccccagcccacttctcagcctcagcctacccctcccaatagcatgccaccctacttgcccaggactcaagctgctggccctgtgtcccagggtaaggcagcaggccaggtgacccctccaacccctcctcagactgctcagccaccccttccagggcccccacctgcagcagtggaaatggcaatgcagattcagagagcagcggagacgcagcgccagatggcccacgtgcaaatttttcaaaggccaatccaacaccagatgcccccgatgactcccatggcccccatgggtatgaacccacctcccatgaccagaggtcccagtgggcatttggagccagggatgggaccgacagggatgcagcaacagccaccctggagccaaggaggattgcctcagccccagcaactacagtctgggatgccaaggccagccatgatgtcagtggcccagcatggtcaacctttgaacatggctccacaaccaggattgggccaggtaggtatcagcccactcaaaccaggcactgtgtctcaacaagccttacaaaaccttttgcggactctcaggtctcccagctctcccctgcagcagcaacaggtgcttagtatccttcacgccaacccccagctgttggctgcattcatcaagcagcgggctgccaagtatgccaactctaatccacaacccatccctgggcagcctggcatgccccaggggcagccagggctacagccacctaccatgccaggtcagcagggggtccactccaatccagccatgcagaacatgaatccaatgcaggcgggcgttcagagggctggcctgccccagcagcaaccacagcagcaactccagccacccatgggagggatgagcccccaggctcagcagatgaacatgaaccacaacaccatgccttcacaattccgagacatcttgagacgacagcaaatgatgcaacagcagcagcaacagggagcagggccaggaataggccctggaatggccaaccataaccagttccagcaaccccaaggagttggctacccaccacagcagcagcagcggatgcagcatcacatgcaacagatgcaacaaggaaatatgggacagataggccagcttccccaggccttgggagcagaggcaggtgccagtctacaggcctatcagcagcgactccttcagcaacagatggggtcccctgttcagcccaaccccatgagcccccagcagcatatgctcccaaatcaggcccagtccccacacctacaaggccagcagatccctaattctctctccaatcaagtgcgctctccccagcctgtcccttctccacggccacagtcccagcccccccactccagtccttccccaaggatgcagcctcagccttctccacaccacgtttccccacagacaagttccccacatcctggactggtagctgcccaggccaaccccatggaacaagggcattttgccagcccggaccagaattcaatgctttctcagcttgctagcaatccaggcatggcaaacctccatggtgcaagcgccacggacctgggactcagcaccgataactcagacttgaattcaaacctctcacagagtacactagacatacactagagacaccttgtagtattttgggagcaaaaaaattattttctcttaacaagactttttgtactgaaaacaatttttttgaatctttcgtagcctaaaagacaattttccttggaacacataagaactgtgcagtagccgtttgtggtttaaagcaaacatgcaagatgaacctgagggatgatagaatacaaagaatatatttttgttatggctggttaccaccagcctttcttcccctttgtgtgtgtggttcaagtgtgcactgggaggaggctgaggcctgtgaagccaaacaatatgctcctgccttgcacctccaataggttttattattttttttaaattaatgaacatatgtaatattaatagttattatttactggtgcagatggttgacatttttccctattttcctcactttatggaagagttaaaacatttctaaaccagaggacaaaaggggttaatgttactttaaaattacattctatatatatataaatatatataaatatatattaaaataccagttttttttctctgggtgcaaagatgttcattcttttaaaaaatgtttaaaaaaaaaaaaaaactgcctttcttcccctcaagtcaacttttgtgctccagaaaattttctattctgtaagtctgagcgtaaaacttcaagtattaaaataatttgtacatgtagagagaaaaatgactttttcaaaaatatacaggggcagctgccaaattgatgtattatatattgtggtttctgtttcttgaaagaatttttttcgttatttttacatctaacaaagtaaaaaaattaaaaagagggtaagaaacgattccggtgggatgattttaacatgcaaaatgtccctgggggtttcttctttgcttgctttcttcctccttaccctaccccccactcacacacacacacacacacacacacacacacacacacacacactttctataaaacttgaaaatagcaaaaaccctcaactgttgtaaatcatgcaattaaagttgattacttataaatatgaactttggatcactgtatagactgttaaatttgatttcttattacctattgttaaataaactgtgtgagacagaca

(19) United States
(12) Patent Application Publication
(10) Pub. No.: US 2025/0115571 A1
(43) Pub. Date: Apr. 10, 2025

(54) OXOPIPERAZINE DERIVATIVES

(71) Applicant: Inthera Bioscience AG, Zurich (CH)

(72) Inventors: Marc LABELLE, Bedford, NH (US); Ulrich KESSLER, Zurich (CH); Valentino CATTORI, Horgen (CH); Cyril COOK, Winnipeg (CA); Ramkrishna Reddy VAKITI, Winnipeg (CA); Kevin R. D. JOHNSON, Winnipeg (CA); Matinder KAUR, Winnipeg (CA); Jean-d'Amour K. TWIBANIRE, Winnipeg (CA); Farman ULLAH, Winnipeg (CA)

(21) Appl. No.: 18/650,360

(22) Filed: Apr. 30, 2024

Related U.S. Application Data
(62) Division of application No. 17/570,959, filed on Jan. 7, 2022, now Pat. No. 12,012,393.
Application No. 17/570,959 is a continuation of application No. 16/891,417, filed on Jun. 3, 2020, now Pat. No. 11,306,068.
Application No. 16/891,417 is a division of application No. 16/222,479, filed on Dec. 17, 2018, now Pat. No. 10,710,975.
(60) Provisional application No. 62/599,336, filed on Dec. 15, 2017.

(30) Foreign Application Priority Data
Feb. 8, 2018 (CH) 00152/18

(51) Int. Cl. C07D 401/06; A61K 45/06; A61P 35/00; A61P 35/02; C07D 401/14; C07D 471/04; C07D 471/10; C07D 491/107; C07D 491/113; C07D 491/20; C07D 495/04; C07D 495/10; C07D 497/10

(52) U.S. Cl. CPC C07D 401/06; A61P 35/00; A61P 35/02; C07D 401/14; C07D 471/04; C07D 471/10; C07D 491/107; C07D 491/113; C07D 491/20; C07D 495/04; C07D 495/10; C07D 497/10; A61K 45/06

(57) ABSTRACT

The present invention relates to novel compounds of formula (I) or formula (Ia)

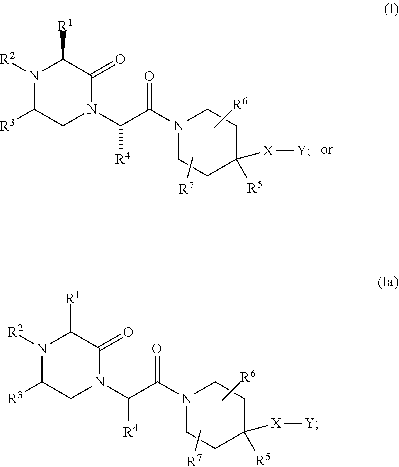

pharmaceutically-acceptable salts, hydrates, solvates, or stereoisomers thereof, and pharmaceutical compositions of these compounds which are useful for preventive and therapeutic use in human and veterinary medicine.

### CROSS-REFERENCE TO RELATED APPLICATIONS

This application is a division of U.S. application Ser. No. 17/570,959, filed Jan. 7, 2022, which is a continuation of U.S. application Ser. No. 16/891,417, filed Jun. 3, 2020 (now U.S. Pat. No. 11,306,068, issued Apr. 19, 2022), which is a division of U.S. application Ser. No. 16/222,479, filed Dec. 17, 2018 (now U.S. Pat. No. 10,710,975, issued Jul. 14, 2020), which claims priority to, and the benefit of U.S. Application No. 62/599,336, filed Dec. 15, 2017 and Swiss Application No. 00152/18, filed Feb. 8, 2018, the entire contents of each of which are incorporated herein by reference.

### INCORPORATION OF THE SEQUENCE LISTING

The instant application contains a Sequence Listing which has been submitted in XML format via Patent Center and is hereby incorporated by reference in its entirety. The contents of the electronic sequence listing (NTHR_001_D02US_SeqList_ST26.xml; Size: 13,962 bytes; and Date of Creation: Apr. 29, 2024) are herein incorporated by reference in its entirety.

### Field of the Invention

The present invention relates to novel compounds of formula (I) or formula (Ia):

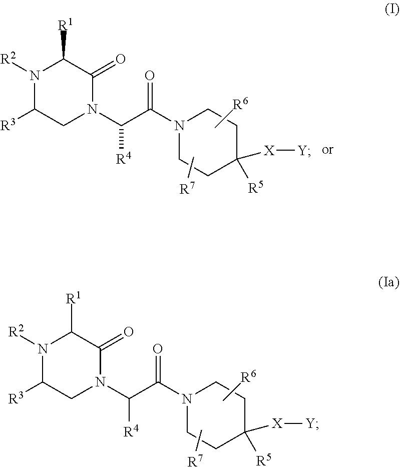

pharmaceutically-acceptable salts, hydrates, solvates, or stereoisomers thereof, and pharmaceutical compositions of these compounds which are useful for preventive and therapeutic use in human and veterinary medicine.

### BACKGROUND

Despite the ever increasing number of cancer therapies in general, and combination cancer therapies in particular, cancer is still the third most common cause of death worldwide after cardiovascular diseases and infectious/parasitic diseases; in absolute numbers, this corresponds to 7.6 million deaths (ca. 13% of all deaths) in any given year. The World Health Organization (WHO) estimates deaths due to cancer to increase to 13.1 million by 2030, while the American Cancer Society expects over 1,685,210 new cancer cases diagnosed and 595,690 cancer deaths in the U.S. in 2016. A 2012 survey by McMillan Cancer Support in the U.K. has revealed that the median survival time of cancer patients overall has increased from 1 year to 6 years since the 1970s. However, for many cancers including esophageal-, stomach-, lung-, brain- and pancreatic cancer, median survival has barely improved, remaining less than one year. These statistics illustrate the fact that cancer remains a critical health condition and that there is an urgent need for new anticancer drugs.

### SUMMARY

The present invention relates to novel compounds of formula (Ia). The present invention provides novel compounds according to formula (Ia):

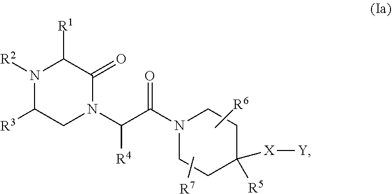

or a pharmaceutically acceptable salt, hydrate, solvate or stereoisomer thereof, wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by cycloalkyl, aryl or heteroaryl, wherein the cycloalkyl, aryl or the heteroaryl is optionally substituted by halogen, C₁₋₄ alkyl or C₃₋₅ cycloalkyl;
R² is selected from H, C(O)R¹⁴, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by cycloalkyl, aryl or heteroaryl, wherein the cycloalkyl, aryl or the heteroaryl is optionally substituted by halogen, C₁₋₄ alkyl or C₃₋₅ cycloalkyl; with the proviso that when R² is C(O)NR¹⁵R¹⁵, both R¹⁵ can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, or C₄₋₇ cycloalkenyl, all optionally substituted by halogen, OR⁸, NR⁸R¹¹, or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by cycloalkyl, aryl or heteroaryl, wherein the cycloalkyl, aryl or the heteroaryl is optionally substituted by halogen, C₁₋₄ alkyl or C₃₋₅ cycloalkyl;
R⁵ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸; and wherein R⁵ can form a ring with any part of X or Y, wherein the ring optionally contains a carbonyl group;
R⁶ is selected from H, C₁₋₇ alkyl, C₂₋₇ alkenyl, C₂₋₇ alkynyl, C₃₋₇ cycloalkyl, C₄₋₇ cycloakenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; and wherein R⁶ can form a ring with any part of X; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl;
X is selected from a bond, C₁₋₇ alkanediyl, C₂₋₇ alkenediyl, C₂₋₇ alkynediyl, C₃₋₉ cycloalkanediyl, C₄₋₆ cycloalkenediyl, —O—, C₁₋₃ alkanediyl-O—, —O—C₁₋₇ alkanediyl, —O—C₃₋₉ cycloalkanediyl, C₁₋₃ alkanediyl-O—C₁₋₇ alkanediyl, C₁₋₇ heteroalkanediyl, or —S—C₁₋₇ alkanediyl; and wherein X can form a ring or a polycyclic system with any part of R⁵, R⁶, or Y, wherein the ring optionally contains a carbonyl group;
Y is selected from H, C(O)NR¹⁰R¹², C(O)OR¹⁰, R¹⁰NC(O)NR¹⁰R¹², OC(O)R¹⁰, OC(O)NR¹⁰R¹², S(O)ₙR⁸ wherein n is 0, 1 or 2, SO₂NR¹⁰R¹², NR¹⁰SO₂R¹⁰, NR¹⁰R¹², HNCOR⁸, CN, C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or aryl, heteroaryl wherein the aryl or heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R⁹ is selected from H, halogen, C₁₋₅ alkyl, C₂₋₅ alkenyl, C₂₋₅ alkynyl, C₃₋₅ cycloalkyl, C₁₋₅ alkyl-OR⁸, C₁₋₅ alkyl-SR⁸, C₁₋₅ alkyl-NR⁸R¹¹, C₁₋₅ alkyl-C(O)OR⁸, C₁₋₅ alkyl-C(O)NR⁸R¹¹, C₁₋₅ alkyl-C(O)R¹⁰, CN, C(O)R⁸, C(O)NR⁸R¹¹, C(O)OR⁸, NR⁸C(O)NR⁸R¹¹, OC(O)NR⁸R¹¹, SO₂NR⁸R¹¹, NR⁸SO₂R⁸, OR⁸, NR⁸R¹¹, or S(O)ₙR⁸ wherein n is 0, 1 or 2;
R¹⁰ and R¹² are each independently selected from H, C₁₋₇ alkyl, C₂₋₇ alkenyl, C₂₋₇ alkynyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen, OR⁸, or NR⁸R¹¹;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl; and
each R¹⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.

In another aspect, the present invention relates to novel compounds of formula (I). The present invention provides novel compounds according to formula (I):

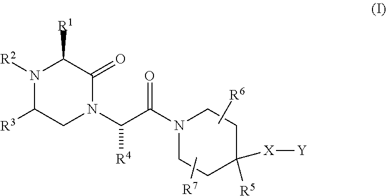

or a pharmaceutically acceptable salt, hydrate, solvate or stereoisomer thereof, wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R² is selected from H, C(O)R¹⁴, C(O)N¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by aryl or heteroaryl, wherein the aryl or the heteroaryl is optionally substituted by halogen, C₁₋₄ alkyl or C₃₋₅ cycloalkyl; with the proviso that when R² is C(O)NR¹⁵R¹⁵, both R¹⁵ can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R⁵ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸; and wherein R⁵ can form a ring with any part of X or Y, wherein the ring optionally contains a carbonyl group;
R⁶ is selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; or C₁₋₃ alkyl substituted by C(O)NR⁸R¹¹; C₁₋₃ alkyl substituted by aryl or heteroaryl, wherein the aryl or the heteroaryl is optionally substituted by halogen, C₁₋₄ alkyl or C₃₋₅ cycloalkyl; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl;
X is selected from a bond, C₁₋₇ alkanediyl, C₂₋₇ alkenediyl, C₂₋₇ alkynediyl, C₃₋₆ cycloalkanediyl, C₄₋₆ cycloalkenediyl, —O—, C₁₋₃ alkanediyl-O—, —O—C₁₋₇ alkanediyl, C₁₋₃ alkanediyl-O—C₁₋₇ alkanediyl, C₁₋₇ heteroalkanediyl, or —S—C₁₋₇ alkanediyl; and wherein X can form a ring with any part of R⁵ or Y, wherein the ring optionally contains a carbonyl group;
Y is selected from H, C(O)NR¹⁰R¹², C(O)OR¹⁰, R¹⁰NC(O)NR¹⁰R¹², OC(O)R¹⁰, OC(O)NR¹⁰R¹², S(O)ₙR⁸ wherein n is 0, 1 or 2, SO₂NR¹⁰R¹², NR¹⁰SO₂R¹⁰, NR¹⁰R¹², HNCOR⁸, CN, C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; aryl, or heteroaryl wherein the aryl or heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R⁹ is selected from H, halogen, C₁₋₅ alkyl, C₂₋₅ alkenyl, C₂₋₅ alkynyl, C₃₋₅ cycloalkyl, C₁₋₅ alkyl-OR⁸, C₁₋₅ alkyl-SR⁸, C₁₋₅ alkyl-NR⁸R¹¹, C₁₋₅ alkyl-C(O)OR⁸, C₁₋₅ alkyl-C(O)NR⁸R¹¹, C₁₋₅ alkyl-C(O)R¹⁰, CN, C(O)R⁸, C(O)NR⁸R¹¹, C(O)OR⁸, NR⁸C(O)NR⁸R¹¹, OC(O)NR⁸R¹¹, SO₂NR⁸R¹¹, NR⁸SO₂R⁸, OR⁸, NR⁸R¹¹, or S(O)ₙR⁸ wherein n is 0, 1 or 2;
R¹⁰ and R¹² are each independently selected from H, C₁₋₇ alkyl, C₂₋₇ alkenyl, C₂₋₇ alkynyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen, OR⁸, or NR⁸R¹¹;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl; and
each R¹⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.

The present invention also relates to pharmaceutical compositions useful for preventive and therapeutic use in human and veterinary medicine comprising compounds of the formula (I) and/or formula (Ia) and pharmaceutically-acceptable salts, hydrates, solvates, or stereoisomers thereof. The present invention is useful in methods for preventing and treating cancer.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to hose described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of compounds disclosed herein, the chemical structures will control.

Other features and advantages of the disclosure will be apparent from the following detailed description and claims.

### BRIEF DESCRIPTION OF THE FIGURES

FIG. 1 is a graph showing tumor growth inhibition in a patient-derived xenograft model of head and neck cancer. NMRI nude mice bearing HN11873 subcutaneous tumors were treated p.o. BID with either vehicle (control) or 30 mg/kg test Compound 57.

FIG. 2A-2F are graphs depicting characteristic 5-day cell proliferation inhibition curves. The figure shows the inhibition curves for the values reported for lymphoma cell lines and Compound 258 (light gray line) in TABLE 5. Concentrations are given in micromol/lt (μM), T₀=day 0 reading (proliferation reference). The cisplatin (quality control) inhibition curve is shown in dark grey.

FIG. 3 is a graph showing the tumor size development in a castration-resistant patient-derived xenograft mouse model of prostate cancer at various concentrations of Compound 258 and Compound 284, compared to standard-of-care treatment (Enzalutamide). The arrow indicates a concentration switch from Compound 258 10 mg/kg to 3 mg/kg at day 51.

FIG. 4 is a graph showing mice body weight development in a castration-resistant patient-derived xenograft mouse model of prostate cancer at various concentration of Compound 258 and Compound 284, compared to standard-of-care treatment (Enzalutamide). The arrow indicates a concentration switch from Compound 258 10 mg/kg to 3 mg/kg at day 51.

FIG. 5 is a graph showing tumor size development in a hormone-resistant cell-derived xenograft mouse model of prostate cancer (DU-145 cells) at various concentrations of Compound 258, Compound 279, Compound 253 and Compound 284.

FIG. 6 is a graph showing mice body weight development in a hormone-resistant cell-derived xenograft mouse model of prostate cancer (DU-145 cells) at various concentrations of Compound 258, Compound 279, Compound 253 and Compound 284.

FIG. 7 is a graph showing tumor size development in a colorectal cancer cell-derived xenograft mouse model (HCT116 cells) at various concentrations of Compound 258, Compound 279 and Compound 284, compared to standard-of-care treatment (Avastin, also called bevacizumab).

FIG. 8 is a graph showing mice body weight development in a colorectal cancer cell-derived xenograft mouse model (HCT116 cells) at various concentrations of Compound 258, Compound 279 and Compound 284, compared to standard-of-care treatment (Avastin).

FIG. 9 is a graph showing the tumor size development in a gastric cancer cell-derived xenograft mouse model (MKN45 cells) at various concentrations of Compound 258, Compound 253 and Compound 284, compared to standard-of-care treatment (Paclitaxel).

FIG. 10 is a graph showing mice body weight development in a gastric cancer cell-derived xenograft mouse model (MKN45 cells) at various concentrations of Compound 258, Compound 253 and Compound 284, compared to standard-of-care treatment (Paclitaxel).

FIG. 11 is a graph showing tumor size development in an HPV-positive cervical cancer cell-derived xenograft mouse model (SiHa cells) at various concentrations of Compound 248, Compound 273, Compound 318 and Compound 258.

FIG. 12 is a graph showing mice body weight development in an HPV-positive cervical cancer cell-derived xenograft mouse model (SiHa cells) at various concentrations of Compound 248, Compound 273, Compound 318 and Compound 258.

FIGS. 13A and 13B are graphs showing luciferase activity in a MOLM13-Luc mouse model for acute myeloid leukemia tumor spread. (A) Mean in vivo luciferase activity (photons/s) profile (whole body imaging): test compound is Compound 258, administered at 1, 3 and 6 mg/kg, displayed versus the corresponding vehicle control group. Data are displayed as mean values+/−SEM. (B) Luciferase activity (photons/s), measured in vivo on Day 19 (whole body imaging at necropsy): test compound is Compound 258, administered at 1, 3 and 6 mg/kg, displayed versus the corresponding vehicle control group. Data are displayed as individual data points together with their corresponding median values and interquartile ranges. P-values were calculated compared to the corresponding vehicle control group and between the 3 mg/kg and 6 mg/kg groups, using the Mann Whitney test and the unpaired t-test (in parentheses) as well as the one-way ANOVA with Dunnett's post test. *=p<0.05; **=p<0.01; ***=p<0.001.

FIG. 14 is a graph showing the mean animal weight (g) profile in a MOLM13-Luc mouse model for acute myeloid leukemia tumor spread. The test compound is Compound 258, administered at 1, 3 and 6 mg/kg, displayed versus the corresponding vehicle control group. Data are displayed as mean values+/−SEM.

FIG. 15 is a graph showing the ex vivo, post-necropsy organ/tissue luciferase activity (Photons/s/mg weight or Photon/s for lymph nodes) in a MOLM13-Luc mouse model for acute myeloid leukemia tumor spread. Test compound is Compound 258, administered at 1, 3 and 6 mg/kg is displayed versus the corresponding Vehicle Control Group, for femur, lumbar spine, peritoneal carcinomatosis (fat tissue) and lymph nodes (both axillary and inguinal). Data are displayed as means+/−SD. P values were calculated compared to the corresponding Vehicle Control Group using the Mann Whitney test.

FIG. 16 is a graph showing the tumor volume development in a patient-derived HPV-positive human head-and-neck squamous cell carcinoma xenograft mouse model for Compound 248 and Compound 282 (both 30 mg/kg, twice a day, administered orally).

FIG. 17 is a graph showing mice body weight development in a patient-derived HPV-positive human head-and-neck squamous cell carcinoma xenograft mouse model for Compound 248 and Compound 282 (both 30 mg/kg, twice a day, administered orally).

FIG. 18 is a graph showing the tumor volume development in a patient-derived HPV-positive human head-and-neck squamous cell carcinoma xenograft mouse model for Compound 57, Compound 248, Compound 282 and Compound 273, at variable dosages (TABLE 23). All sixteen mice treated with Compound 248 and Compound 273 were tumor-free at the end of the observation period.

FIG. 19 is a graph showing mice body weight development in a patient-derived HPV-positive human head-and-neck squamous cell carcinoma xenograft mouse model for Compound 57, Compound 248, Compound 282 and Compound 273, at variable dosages (TABLE 23).

FIG. 20 is a graph showing the tumor volume development in a cell-derived syngeneic mouse model for colorectal carcinoma (CT-26 cells) combined with an immuno-oncology treatment (anti-PD1 antibodies). Compound 258 was administered as single agent and as a combination. Data after day 21 are mean+/−SEM of mice still in the experiment. Only the combination therapies and anti-PD1 have data after day 28 (TABLE 26). Two mice displayed complete regression in the combination groups, hence the huge SEM-values.

FIG. 21 is a graph showing mice body weight development in a cell-derived syngeneic mouse model for colorectal carcinoma (CT-26 cells) combined with an immuno-oncology treatment (anti-PD1 antibodies). Compound 258 was administered as single agent and as a combination. Data after day 21 are mean+/−SEM of mice still in the experiment. Only the combination therapies and anti-PD1 have data after day 28 (TABLE 26).

FIG. 22 is a graph showing gene expression inhibition of three well-characterized androgen receptor (AR)-targets through Compound 258-mediated disruption of p300-CH1/TAZ1-AR signaling in the castration-resistant prostate cancer cell line LNCaP. Prostate-specific antigen (PSA/KLK3); transmembrane serine protease 2 (TMPRSS2); and prostein (SLC45A3) gene expression was measured in 4-hour dihydrotestosterone—stimulated cells (DHT, 100 nM) and compared to untreated cells. 300 nM Compound 258 was added concomitantly to DHT. Treatment with Compound 258 resulted in complete repression of PSA stimulation, and 85%, respectively 80% repression of TMPRSS2 and SLC45A3 stimulation.

FIG. 23 is a graph showing serum prostate-specific antigen (PSA) levels in a castration resistant prostate cancer (CRPC) patient-derived xenograft mouse model. Serum levels were determined in five mice that still had detectable tumors at experiment termination (following a 19 day treatment period, blood samples taken 3 h after the last dose was applied, FIG. 3, numbering on the x-axis of FIG. 23). Two mice were treated daily with 10 and 3 mg/kg and three mice daily with 6 mg/kg Compound 258 (FIG. 3). Minimal relative expected PSA-levels were calculated based on minimal PSA/tumor size ratio of vehicle-treated mice. All five mice had a clear reduction of the expected serum PSA levels.

FIG. 24 is a graph showing tumor Vascular Endothelial Growth Factor A (VEGF) protein levels in HCT-116 and MKN45 colorectal/gastric cancer cell-derived xenograft mouse model after approximately 4 and 3 weeks, respectively, of treatment with 3 mg/kg or 6 mg/kg Compound 258 (FIGS. 7 and 9). In accordance with the proposed mode of action of Compound 258, the p300/CBP-HIF1alpha transcriptional complex was disturbed, resulting in VEGF protein levels which were significantly reduced upon Compound 258-treatment. The effect is more evident in the HCT-116 than in the MKN45 xenograft, reflecting the higher VEGF-dependence of HCT-116 xenograft vascularization (described in Dang et al. Cancer Res 2008; 68(6):1872-80).

FIG. 25 is a pair of Western blots of Compound 258-treated HPV16-positive cervical cancer CaSki cells. The figure depicts a characteristic rescue of p53 protein expression and p53 lysine 382 acetylation (K382Ac-p53) after Compound 258-mediated inhibition of p300/CBP-HPVE6-p53 protein-protein-interactions. Cells were treated with the indicated concentrations (nM) for 72 h. Induction of p53 protein above baseline is evident at 7 nM already, acetylation of p53 lysine 382 is detectable at 20 nM. Equivalent amounts of protein were loaded on the blot and the loading quantity assessed by total protein detection of the same blot on a Bio-Rad ChemiDoc Touch imager.

### DETAILED DESCRIPTION

The present invention is directed to a series of compounds having strong activities against a broad variety of tumor types, including, but not limited to, prostate, colon, head-and-neck and cervical cancer as well as hematological malignancies.

The present invention is directed to a series of compounds having a strong activity as p300/CBP inhibitors, including stereoisomers, tautomers, pharmaceutically acceptable salts and prodrugs thereof, and the use of such compounds to treat p300/CBP-related conditions or diseases, such as cancer.

Exemplary conditions which can be treated with the disclosed compounds include cancer. The cancer types which can be treated include, but are not limited to, prostate cancer, renal cancer, pancreatic cancer, liver cancer, breast cancer, gastric cancer, colorectal cancer, cervical cancer, ovarian cancer, head-and-neck cancer, esophageal cancer, leukemia, lymphoma, lung cancer, brain cancer, cancer of the central nervous system and skin cancer.

The invention provides pharmaceutical compositions of the described compounds, comprising the described compounds and pharmaceutically acceptable carriers, diluents or excipients.

The invention provides pharmaceutical compositions of the described compounds, wherein the compounds are administered in combination with one or more anti-cancer treatments or anti-cancer therapeutic agents. In one aspect, the pharmaceutical composition consists of the combination of one of the compounds with an immune checkpoint inhibitor of programmed cell death protein 1 (PD-1).

### Definitions

The following are definitions of terms used in present application. The initial definition provided for a group or term herein applies to that group or term throughout the description and the claims, individually or as part of another group, unless otherwise indicated.

The term “alkyl” as used herein refers to a saturated straight or branched chain group of carbon atoms derived from an alkane by the removal of one hydrogen atom. C₁₋₃ alkyl includes, but is not limited to, for example methyl, ethyl, n-propyl, i-propyl. C₁₋₄ alkyl comprises for example methyl, ethyl, n-propyl, i-propyl, n-butyl, i-butyl, tert-butyl. C₁₋₅ alkyl comprises for example methyl, ethyl, n-propyl, i-propyl, n-butyl, i-butyl, tert-butyl, n-pentyl, C₁₋₇ alkyl comprises for example methyl, ethyl, n-propyl, i-propyl, n-butyl, i-butyl, tert-butyl, n-pentyl, n-hexyl or n-heptyl. The alkyl groups of this invention can be optionally substituted.

The term “C₂₋₅ alkenyl” and “C₂₋₇ alkenyl” as used herein refers to straight or branched chain hydrocarbon groups having 2 to 5 carbon atoms and 2 to 7 carbon atoms, respectively and at least one double bond.

The term “C₂₋₅ alkynyl” and “C₂₋₇ alkynyl” as used herein refers to straight or branched chain hydrocarbon groups having 2 to 5 carbon atoms and 2 to 7 carbon atoms, respectively and at least one triple bond.

The term “C₃₋₇ cycloalkyl” and “C₃₋₅ cycloalkyl” as used herein refers to a monovalent saturated cyclic or bicyclic hydrocarbon group of 3-7 or 3-5 carbons, respectively derived from a cycloalkane by the removal of a single hydrogen atom. “C₃₋₅ cycloalkyl” includes, but is not limited to, cyclopropyl, cyclobutyl, and cyclopentyl. “C₃₋₇ cycloalkyl” includes, but is not limited to, cyclopropyl, cyclobutyl, cyclopentyl, cyclohexyl and cycloheptyl. The term “C₃₋₇ cycloalkyl” and “C₃₋₅ cycloalkyl” as used herein also includes cycloalkyl groups that comprise a C₁₋₃-alkyl radical. Examples of such “C₃₋₇ cycloalkyl” groups comprise cyclopropylmethyl, 2-cyclopropylethyl, cyclobutylmethyl, 2-cyclobutylethyl, cyclopentylmethyl, 2-cyclopentylethyl. Examples of such “C₃₋₅ cycloalkyl” groups comprise cyclopropylmethyl, 2-cyclopropylethyl, cyclobutylmethyl. Cycloalkyl groups of this invention can be optionally substituted. Substitutents can be e.g. halogen, C₁₋₄ alkyl or C₃₋₅ cycloalkyl.

The term “C₄₋₇ cycloalkenyl” as used herein refers to a monovalent cyclic or bicyclic hydrocarbon group of 4-7 carbons having at least one double bond, derived from a cycloalkene by the removal of a single hydrogen atom. The term “C₄₋₇ cycloalkenyl” as used herein also includes cycloalkenyl groups that comprise a C₁₋₃-alkyl radical.

The term “C₁₋₃ alkanediyl”, “C₁₋₆ alkanediyl” and “C₁₋₇ alkanediyl” as used herein refers to a diradical of a saturated straight or branched chain hydrocarbon group, having 1 to 3, 1 to 6 carbon and 1 to 7 carbon atoms, respectively. Examples of alkanediyl groups include methane-diyl, ethane-1,2-diyl, and the like.

The term “C₂₋₆ alkenediyl” and “C₂₋₇ alkenediyl” as used herein refers to a diradical of a straight or branched chain hydrocarbon groups having 2 to 6 carbon atoms and 2 to 7 carbon atoms, respectively and at least one double bond. Examples of alkenediyl groups include ethene-1,2-diyl and the like.

The term “C₂₋₆ alkynediyl” and “C₂₋₇ alkynediyl” as used herein refers to a diradical of a straight or branched chain hydrocarbon groups having 2 to 6 carbon atoms and 2 to 7 carbon atoms, respectively and at least one triple bond. Examples of alkynediyl groups include ethine-1,2-diyl and the like.

The term “C₃₋₆ cycloalkanediyl” as used herein refers to a diradical saturated cyclic or bicyclic hydrocarbon group of 3-6 carbons.

The term “C₃₋₆ cycloalkenediyl” as used herein refers to a diradical cyclic or bicyclic hydrocarbon group of 3-6 carbons having at least one double bond.

The term “heteroalkyl” or “heteroalkanediyl” as used herein refers to an alkyl radical or an alkanediyl radical as defined herein wherein one, two, three or four hydrogen atoms have been replaced with a substituent independently selected from the group consisting of OH, NH₂ and halogen. Representative examples include, but are not limited to, 2-hydroxyethyl, 2-hydroxypropyl, 3-hydroxypropyl, 2-hydroxy-1-hydroxymethylethyl, 2-hydroxy-1-methylethyl, 2,3-dihydroxypropyl, 1-hydroxymethylethyl, 3-hydroxybutyl, 2,3-dihydroxybutyl, 1-hydroxy-2-methylpropyl, 3-hydroxy-1-(2-hydroxyethyl)-propyl, 2-hydroxy-1-methylpropyl, 1,1,1-trifluoroethyl, 2,2,3,3-tetrafluoropropyl.

The term “aryl” as used herein refers to a mono- or bicyclic carbocyclic ring system having one or two aromatic rings. The aryl group can also be fused to a cyclohexane, cyclohexene, cyclopentane, or cyclopentene ring or to a cyclohexane, cyclohexene, cyclopentane, or cyclopentene ring comprising a carbonyl group. Thus the aryl group includes e.g. indane or mono-oxo substituted indane rings. The aryl groups of this invention can be optionally substituted as further described below. A preferred aryl group and optionally substituted aryl group, respectively of this invention is a phenyl group or substituted phenyl group. Substitutents can be e.g. halogen, C₁₋₄ alkyl or C₃₋₅ cycloalkyl.

The term “heteroaryl” as used herein refers to substituted and unsubstituted aromatic 5-, or 6-membered monocyclic groups and 9- or 10-membered bicyclic groups, which have at least one heteroatom (O, S or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. Heteroaryl groups must include at least one fully aromatic ring but the other fused ring or rings may be aromatic or non-aromatic. The heteroaryl group may be attached at any available nitrogen or carbon atom of any ring. Heteroaryl groups of this invention can be optionally substituted as further described below. Usually, a heteroaryl group and optionally substituted heteroaryl group, respectively of this invention is selected from the group consisting of substituted and/or unsubstituted aromatic 5-, or 6-membered monocyclic groups, which have at least one heteroatom (O, S or N), preferably one heteroatom (O, S or N), more preferably one O or N in the ring, even more preferably two N in the ring. A preferred heteroaryl group and optionally substituted heteroaryl group, respectively of this invention is selected from the group consisting of a pyridinyl group, a substituted pyridinyl group, a imidazole group, a substituted imidazole group, a pyrazole group, a substituted pyrazole group, a triazole group, a substituted triazole group, a benzimidazole group and a substituted benzimidazole group. More preferably a substituted pyridinyl group, a pyridinyl group, a triazole group, a substituted triazole group, a imidazole group, and/or a substituted imidazole group, is used as heteroaryl group in the present invention.

Most preferably a substituted pyridinyl group, a pyridinyl group, an imidazole group, and/or a substituted imidazole group, is used as heteroaryl group in the present invention. Substitutents can be e.g. halogen, C₁₋₄ alkyl or C₃₋₅ cycloalkyl.

The term “S-aryl” as used herein refers to a radical —SR where R is an aryl as defined herein.

The term “O-aryl” as used herein refers to a radical —OR where R is an aryl as defined herein.

The term “S-heteroaryl” as used herein refers to a radical —SR where R is an heteroaryl as defined herein.

The term “O-heteroaryl” as used herein refers to a radical —OR where R is an heteroaryl as defined herein.

The term “C₁₋₃ alkyl-aryl” as used herein refers to a radical of C₁₋₃ alkyl as defined herein to which an aryl group as defined herein is bonded at any carbon of the alkyl.

The term “C₁₋₃ alkyl-heteroaryl” as used herein refers to a radical of C₁₋₃ alkyl as defined herein to which a heteroaryl group as defined herein is bonded at any carbon of the alkyl.

The terms “halo” or “halogen” as used herein refers to F, Cl, Br, or I and is preferably F, Cl, or Br.

### Compounds of the Present Disclosure

In some aspects, the present disclosure relates to a compound of Formula (Ia):

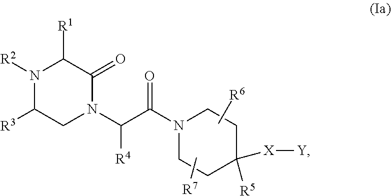

or a pharmaceutically acceptable salt, hydrate, solvate or stereoisomer thereof, wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R² is selected from H, C(O)R¹⁴, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by aryl or heteroaryl, wherein the aryl or the heteroaryl is optionally substituted by halogen, C₁₋₄ alkyl or C₃₋₅ cycloalkyl; with the proviso that when R² is C(O)NR¹⁵R¹⁵, both R¹⁵ can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, or C₄₋₇ cycloalkenyl, all optionally substituted by halogen, OR⁸, NR⁸R¹¹, or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R⁵ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸; and wherein R⁵ can form a ring with any part of X or Y, wherein the ring optionally contains a carbonyl group;
R⁶ is selected from H, C₁₋₇ alkyl, C₂₋₇ alkenyl, C₂₋₇ alkynyl, C₃₋₇ cycloalkyl, C₄₋₇ cycloakenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; and wherein R⁶ can form a ring with any part of X; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl;
X is selected from a bond, C₁₋₇ alkanediyl, C₂₋₇ alkenediyl, C₂₋₇ alkynediyl, C₃₋₉ cycloalkanediyl, C₄₋₆ cycloalkenediyl, —O—, C₁₋₃ alkanediyl-O—, —O—C₁₋₇ alkanediyl, —O—C₃₋₉ cycloalkanediyl, C₁₋₃ alkanediyl-O—C₁₋₇ alkanediyl, C₁₋₇ heteroalkanediyl, or —S—C₁₋₇ alkanediyl; and wherein X can form a ring or a polycyclic system with any part of R⁵, R⁶, or Y, wherein the ring optionally contains a carbonyl group;
Y is selected from H, C(O)NR¹⁰R¹², C(O)OR¹⁰, R¹⁰NC(O)NR¹⁰R¹², OC(O)R¹⁰, OC(O)NR¹⁰R¹², S(O)ₙR⁸ wherein n is 0, 1 or 2, SO₂NR¹⁰R¹², NR¹⁰SO₂R¹⁰, NR¹⁰R¹², HNCOR⁸, CN, C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or aryl, heteroaryl wherein the aryl or heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring optionally substituted by R⁹ or R¹⁴; wherein the ring contains the N of NR¹⁰R¹² and optionally one further heteroatom selected from O and N, wherein if the one further heteroatom is N, it is optionally substituted by R⁸;
R⁹ is selected from H, halogen, C₁₋₅ alkyl, C₂₋₅ alkenyl, C₂₋₅ alkynyl, C₃₋₅ cycloalkyl, C₁₋₅ alkyl-OR⁸, C₁₋₅ alkyl-SR⁸, C₁₋₅ alkyl-NR⁸R¹¹, C₁₋₅ alkyl-C(O)OR⁸, C₁₋₅ alkyl-C(O)NR⁸R¹¹, C₁₋₅ alkyl-C(O)R¹⁰, CN, C(O)R⁸, C(O)NR⁸R¹¹, C(O)OR⁸, NR⁸C(O)NR⁸R¹¹, OC(O)NR⁸R¹¹, SO₂NR⁸R¹¹, NR⁸SO₂R⁸, OR⁸, NR⁸R¹¹, or S(O)ₙR⁸ wherein n is 0, 1 or 2;
R¹⁰ and R¹² are each independently selected from H, C₁₋₇ alkyl, C₂₋₇ alkenyl, C₂₋₇ alkynyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, C₁₋₃ alkyl-heteroaryl, all these groups optionally substituted by halogen, OR⁸, or NR⁸R¹¹;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl; and
each R¹⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.

In some aspects, the present disclosure relates to a compound of Formula (I):

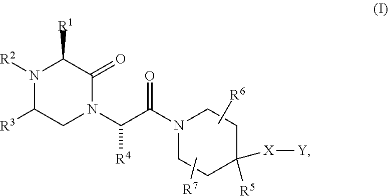

or a pharmaceutically acceptable salt, hydrate, solvate or stereoisomer thereof, wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R² is selected from H, C(O)R¹⁴, C(O)NR¹⁵R¹⁵, C(O)OR⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by aryl or heteroaryl, wherein the aryl or the heteroaryl is optionally substituted by halogen, C₁₋₄ alkyl or C₃₋₅ cycloalkyl; with the proviso that when R² is C(O)NR¹⁵R¹⁵, both R¹⁵ can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, or C₄₋₇ cycloalkenyl, all optionally substituted by halogen, OR⁸, NR⁸R¹¹, or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R⁵ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸; and wherein R⁵ can form a ring with any part of X or Y, wherein the ring optionally contains a carbonyl group;
R⁶ is selected from H, C₁₋₇ alkyl, C₂₋₇ alkenyl, C₂₋₇ alkynyl, C₃₋₇ cycloalkyl, C₄₋₇ cycloakenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl;
X is selected from a bond, C₁₋₇ alkanediyl, C₂₋₇ alkenediyl, C₂₋₇ alkynediyl, C₃₋₆ cycloalkanediyl, C₄₋₆ cycloalkenediyl, —O—, C₁₋₃ alkanediyl-O—, —O—C₁₋₇ alkanediyl, C₁₋₃ alkanediyl-O—C₁₋₇ alkanediyl, C₁₋₇ heteroalkanediyl, or —S—C₁₋₇ alkanediyl; and wherein X can form a ring with any part of R⁵ or Y, wherein the ring optionally contains a carbonyl group;
Y is selected from H, C(O)NR¹⁰R¹², C(O)OR¹⁰, R¹⁰NC(O)NR¹⁰R¹², OC(O)R¹⁰, OC(O)NR¹⁰R¹², S(O)ₙR⁸ wherein n is 0, 1 or 2, SO₂NR¹⁰R¹², NR¹⁰SO₂R¹⁰, NR¹⁰R¹², HNCOR⁸, CN, C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or aryl, heteroaryl wherein the aryl or heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R⁹ is selected from H, halogen, C₁₋₅ alkyl, C₂₋₅ alkenyl, C₂₋₅ alkynyl, C₃₋₅ cycloalkyl, C₁₋₅ alkyl-OR⁸, C₁₋₅ alkyl-SR⁸, C₁₋₅ alkyl-NR⁸R¹¹, C₁₋₅ alkyl-C(O)OR⁸, C₁₋₅ alkyl-C(O)NR⁸R¹¹, C₁₋₅ alkyl-C(O)R¹⁰, CN, C(O)R⁸, C(O)NR⁸R¹¹, C(O)OR⁸, NR⁸C(O)NR⁸R¹¹, OC(O)NR⁸R¹¹, SO₂NR⁸R¹¹, NR⁸SO₂R⁸, OR⁸, NR⁸R¹¹, or S(O)ₙR⁸ wherein n is 0, 1 or 2;
R¹⁰ and R¹² are each independently selected from H, C₁₋₇ alkyl, C₂₋₇ alkenyl, C₂₋₇ alkynyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen, OR⁸, or NR⁸R¹¹;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl; and
each R¹⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.

It is understood that, for a compound of Formula (I) or Formula (Ia), R¹, R², R³, R⁴, R⁵, R⁶, R⁷, R⁸, R⁹, R¹⁰, R¹¹, R¹², R¹³, R¹⁴, R¹⁵, X, and Y can each be, where applicable, selected from the groups described herein, and any group described herein for any of R¹, R², R³, R⁴, R⁵, R⁶, R⁷, R⁸, R⁹, R¹⁰, R¹¹, R¹², R¹³, R¹⁴, R¹⁵, X, and Y can be combined, where applicable, with any group described herein for one or more of the remainder of R¹, R², R³, R⁴, R⁵, R⁶, R⁷, R⁸, R⁹, R¹⁰, R¹¹, R¹², R¹³, R⁴, R¹⁵, X, and Y.

In some embodiments, R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by cycloalkyl, aryl or heteroaryl, wherein the cycloalkyl, aryl or the heteroaryl is optionally substituted by halogen, C₁₋₄ alkyl or C₃₋₅ cycloalkyl

In some embodiments, R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl.

In some embodiments, R¹ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl.

In some embodiments, R¹ is selected from C₂₋₇ alkyl, C₃₋₇ cycloalkyl, or C₁₋₃ alkyl substituted by aryl or heteroaryl, wherein the aryl or the heteroaryl is optionally substituted by halogen, C₁₋₄ alkyl or C₃₋₅ cycloalkyl.

In some embodiments, R¹ is selected from C₃₋₇ alkyl, C₃₋₇ cycloalkyl, or C₁₋₃ alkyl substituted by aryl or heteroaryl.

In some embodiments, R¹ is H.

In some embodiments, R¹ is C₃₋₇ cycloalkyl.

In some embodiments, R¹ is selected from cyclopropyl, cyclobutyl, cyclopentyl, cyclohexyl or cycloheptyl.

In some embodiments, R¹ is selected from cyclopropyl or cyclohexyl.

In some embodiments, R¹ is cyclopropyl.

In some embodiments, R¹ is cyclohexyl.

In some embodiments, R¹ is C₁₋₇ alkyl.

In some embodiments, R¹ is C₂₋₇ alkyl.

In some embodiments, R¹ is C₃₋₇ alkyl.

In some embodiments, R¹ is selected from methyl, ethyl, propyl, isopropyl, butyl, sec-butyl, isobutyl, or tert-butyl.

In some embodiments, R¹ is selected from ethyl, propyl, isopropyl, butyl, sec-butyl, isobutyl, or tert-butyl.

In some embodiments, R¹ is selected from propyl, isopropyl, butyl, sec-butyl, isobutyl, or tert-butyl.

In some embodiments, R¹ is isobutyl.

In some embodiments, R¹ is C₁₋₃ alkyl substituted by cycloalkyl.

In some embodiments, R¹ is methyl, ethyl, or propyl substituted by cyclopropyl, cyclobutyl, cyclopentyl, cyclohexyl or cycloheptyl.

In some embodiments, R¹ is ethyl or propyl substituted by cyclopropyl or cyclohexyl.

In some embodiments, R¹ is C₁₋₃ alkyl substituted by aryl or heteroaryl.

In some embodiments, R¹ is methyl, ethyl, or propyl substituted by phenyl, imidazole, pyridine, or triazole.

In some embodiments, R¹ is ethyl or propyl substituted by phenyl or pyridine.

In some embodiments, R¹ is ethyl substituted by phenyl.

In some embodiments, R¹ is

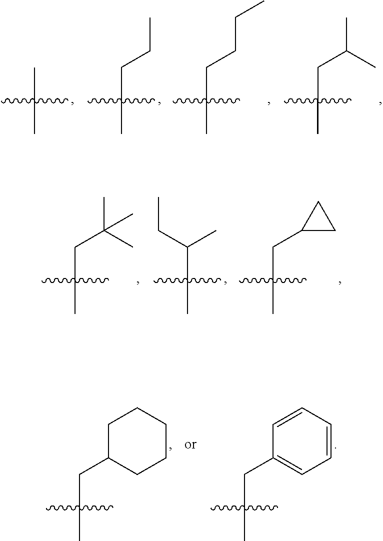

In some embodiments, R¹ is

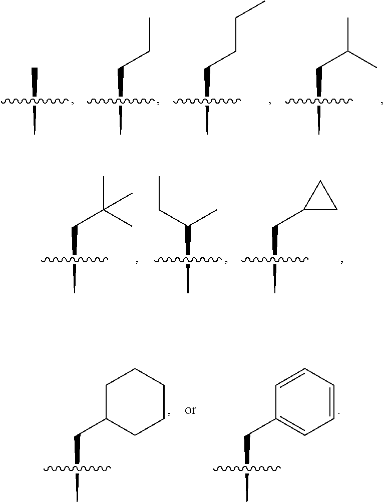

In some embodiments, R¹ is

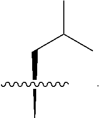

In some embodiments, R² is selected from H, C(O)R¹, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by cycloalkyl, aryl or heteroaryl, wherein the cycloalkyl, aryl or the heteroaryl is optionally substituted by halogen, C₁₋₄ alkyl or C₃₋₅ cycloalkyl; with the proviso that when R² is C(O)NR¹⁵R¹⁵, both R¹⁵ can form a ring wherein the ring contains the N of NR¹⁵R¹⁵ and optionally one further heteroatom selected from O and N, wherein if the one further heteroatom is N, it is optionally substituted by R⁸;

In some embodiments, R² is selected from H, C(O)R¹⁴, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by aryl or heteroaryl, wherein the aryl or the heteroaryl is optionally substituted by halogen, C₁₋₄ alkyl or C₃₋₅ cycloalkyl; with the proviso that when R² is C(O)NR¹⁵R¹⁵, both R¹⁵ can form a ring wherein the ring contains the N of NR¹⁵R¹⁵ and optionally one further heteroatom selected from O and N, wherein if the one further heteroatom is N, it is optionally substituted by R⁸;

In some embodiments, R² is selected from H, C(O)R¹⁴, C(O)OR¹⁵, C₁₋₇ alkyl, C₃₋₇ cycloalkyl, C₁₋₃ alkanediyl-O—C₁₋₃ alkanediyl-O—C₁₋₃ alkanediyl, C₁₋₅ alkyl-OR⁸, C₁₋₅ alkyl-NHCOR¹³, or C₁₋₃ alkyl substituted by aryl, wherein the aryl is optionally substituted by halogen, C₁₋₄ alkyl or C₃₋₅ cycloalkyl.

In some embodiments, R² is selected from H, C(O)R¹⁴, C₁₋₇ alkyl, C₃₋₇ cycloalkyl, C₁₋₅ alkyl-OR⁸, C₁₋₅ alkyl-NHCOR¹³, or C₁₋₃ alkyl substituted by aryl, wherein the aryl is optionally substituted by halogen, C₁₋₄ alkyl or C₃₋₅ cycloalkyl.

In some embodiments, R² is selected from H, C(O)R¹⁴, wherein R¹⁴ is C₁₋₇ alkyl; C₁₋₇ alkyl, C₃₋₇ cycloalkyl, C₁₋₅ alkyl-OR⁸; C₁₋₅ alkyl-NHCOR¹³, wherein R¹³ is pentylamino-5-oxopentyl-7-thia-2,4-diazabicyclo[3.3.0]octan-3-one; or C₁₋₃ alkyl substituted by aryl, wherein the aryl is optionally substituted by halogen, C₁₋₄ alkyl or C₃₋₅ cycloalkyl.

In some embodiments, R² is selected from H, C(O)R¹⁴, wherein R¹⁴ is C₁₋₇ alkyl; C₁₋₇ alkyl, C₃₋₇ cycloalkyl, C₁₋₅ alkyl-OR⁸, wherein R⁸ is C₁₋₇ alkyl; C₁₋₅ alkyl-NHCOR¹³, wherein R¹³ is pentylamino-5-oxopentyl-7-thia-2,4-diazabicyclo[3.3.0]octan-3-one; or C₁₋₃ alkyl substituted by aryl, wherein the aryl is optionally substituted by halogen, C₁₋₄ alkyl or C₃₋₅ cycloalkyl.

In some embodiments, R² is H.

In some embodiments, R² is C₁₋₇ alkyl.

In some embodiments, R² is selected from methyl, ethyl, propyl, isopropyl, butyl, sec-butyl, isobutyl, or tert-butyl.

In some embodiments, R² is selected from methyl, ethyl, or propyl.

In some embodiments, R² is C(O)R⁴, and R¹⁴ is methyl, ethyl, propyl, isopropyl, butyl, sec-butyl, isobutyl, or tert-butyl.

In some embodiments, R² is C(O)NR¹⁵R¹⁵, wherein each R⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.

In some embodiments, R² is C(O)NR¹⁵R¹⁵, wherein each R⁵ is independently selected from methyl, ethyl, propyl, isopropyl, butyl, sec-butyl, isobutyl, or tert-butyl.

In some embodiments, R² is C(O)NR¹⁵R¹⁵, wherein each R⁵ is independently selected from methyl or ethyl.

In some embodiments, R² is C(O)NR¹⁵R¹⁵, wherein each R¹⁵ is methyl.

In some embodiments, R² is C₃₋₇ cycloalkyl.

In some embodiments, R² is selected from cyclopropyl, cyclobutyl, cyclopentyl, cyclohexyl or cycloheptyl.

In some embodiments, R² is cyclopropyl.

In some embodiments, R² is C₁₋₅ alkyl-OR⁸, wherein R⁸ is C₁₋₇ alkyl

In some embodiments, R² is methyl-OR⁸, ethyl-OR⁸, propyl-OR⁸, or butyl-OR⁸ wherein R⁸ is methyl, ethyl, propyl, or butyl.

In some embodiments, R² is ethyl-OR⁸ wherein R⁸ is methyl, ethyl, propyl, or butyl.

In some embodiments, R² is ethyl-OR⁸ wherein R⁸ is methyl.

In some embodiments, R² is C₁₋₃ alkyl substituted by cycloalkyl.

In some embodiments, R² is methyl, ethyl, or propyl substituted by cyclopropyl, cyclobutyl, cyclopentyl, cyclohexyl or cycloheptyl.

In some embodiments, R² is ethyl or propyl substituted by cyclopropyl or cyclohexyl.

In some embodiments, R² is ethyl substituted by cyclopropyl.

In some embodiments, R² is C₁₋₃ alkyl substituted by aryl or heteroaryl.

In some embodiments, R² is methyl, ethyl, or propyl substituted by phenyl, imidazole, pyridine, or triazole.

In some embodiments, R² is ethyl or propyl substituted by phenyl or pyridine.

In some embodiments, R² is ethyl substituted by phenyl.

In some embodiments, R² is C₁₋₃ alkyl substituted by aryl or heteroaryl, wherein the aryl or heteroaryl is optionally substituted by halogen, C₁₋₄ alkyl or C₃₋₅ cycloalkyl.

In some embodiments, R² is methyl, ethyl, or propyl substituted by phenyl, imidazole, pyridine, or triazole substituted by fluoro, iodo, or bromo.

In some embodiments, R² is ethyl or propyl substituted by phenyl or pyridine substituted fluoro, iodo, or bromo.

In some embodiments, R² is ethyl substituted by phenyl substituted fluoro.

In some embodiments, R² is

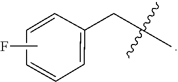

In some embodiments, R² is C₁₋₅ alkyl-NHCOR¹³, wherein R¹³ is pentylamino-5-oxopentyl-7-thia-2,4-diazabicyclo[3.3.0]octan-3-one.

In some embodiments, R² is methyl-NHCOR¹³, ethyl-NHCOR¹³, propyl-NHCOR¹³, butyl-NHCOR¹³, or pentyl-NHCOR¹³, wherein R¹³ is pentylamino-5-oxopentyl-7-thia-2,4-diazabicyclo[3.3.0]octan-3-one.

In some embodiments, R² is pentyl-NHCOR¹³, wherein R¹³ is pentylamino-5-oxopentyl-7-thia-2,4-diazabicyclo[3.3.0]octan-3-one.

In some embodiments, R² is

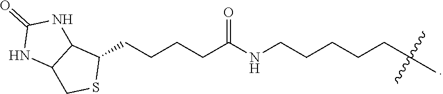 [SMILES: *CCCCCNC(=O)CCCC[C@@H]1SCC2NC(=O)NC21]

In some embodiments, R² is C₁₋₃ alkyl substituted by aryl, wherein the aryl is optionally substituted by halogen, C₁₋₄ alkyl or C₃₋₅ cycloalkyl.

In some embodiments, R² is

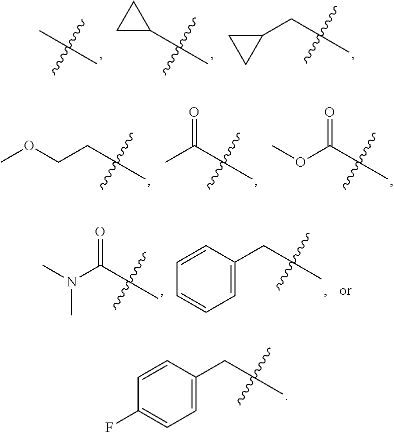

In some embodiments, R³ is selected from H, C₁₋₇ alkyl, C₂₋₇ alkenyl, C₂₋₇ alkynyl, C₃₋₇ cycloalkyl, or C₄₋₇ cycloalkenyl, all optionally substituted by halogen, OR⁸, NR⁸R¹¹, or C₁₋₃ alkyl substituted by aryl or heteroaryl, wherein the aryl or the heteroaryl is optionally substituted by halogen, C₁₋₄ alkyl or C₃₋₅ cycloalkyl.

In some embodiments, R³ is selected from H, C₁₋₇ alkyl, C₂₋₇ alkenyl, C₂₋₇ alkynyl, C₃₋₇ cycloalkyl, or C₄₋₇ cycloalkenyl.

In some embodiments, R³ is C₁₋₇ alkyl.

In some embodiments, R³ is selected from methyl, ethyl, propyl, isopropyl, butyl, sec-butyl, isobutyl, or tert-butyl.

In some embodiments, R³ is C₂₋₇ alkenyl.

In some embodiments, R³ is vinyl.

In some embodiments, R³ is C₃₋₇ cycloalkyl.

In some embodiments, R³ is selected from cyclopropyl, cyclobutyl, cyclopentyl, cyclohexyl or cycloheptyl.

In some embodiments, R³ is H.

In some embodiments, R³ and R⁷ are each independently selected from H, C₁₋₇ alkyl, C₂₋₇ alkenyl, C₂₋₇ alkynyl, C₃₋₇ cycloalkyl, or C₄₋₇ cycloalkenyl, all optionally substituted by halogen, OR⁸, NR⁸R¹¹, or C₁₋₃ alkyl substituted by aryl or heteroaryl, wherein the aryl or the heteroaryl is optionally substituted by halogen, C₁₋₄ alkyl or C₃₋₅ cycloalkyl

In some embodiments, R³ and R⁷ are each independently selected from H, C₁₋₇ alkyl, C₂₋₇ alkenyl, C₂₋₇ alkynyl, C₃₋₇ cycloalkyl, or C₄₋₇ cycloalkenyl.

In some embodiments, R³ and R⁷ are each independently C₁₋₇ alkyl.

In some embodiments, R³ and R⁷ are each independently selected from methyl, ethyl, propyl, isopropyl, butyl, sec-butyl, isobutyl, or tert-butyl.

In some embodiments, R³ and R⁷ are each independently C₂₋₇ alkenyl.

In some embodiments, R³ and R⁷ are each vinyl.

In some embodiments, R³ and R⁷ are each independently C₃₋₇ cycloalkyl.

In some embodiments, R³ and R⁷ are each independently selected from cyclopropyl, cyclobutyl, cyclopentyl, cyclohexyl or cycloheptyl.

In some embodiments, R³ and R⁷ are each H.

In some embodiments, R⁷ is selected from H, C₁₋₇ alkyl, C₂₋₇ alkenyl, C₂₋₇ alkynyl, C₃₋₇ cycloalkyl, or C₄₋₇ cycloalkenyl, all optionally substituted by halogen, OR⁸, NR⁸R¹¹, or C₁₋₃ alkyl substituted by aryl or heteroaryl, wherein the aryl or the heteroaryl is optionally substituted by halogen, C₁₋₄ alkyl or C₃₋₅ cycloalkyl.

In some embodiments, R⁷ is selected from H, C₁₋₇ alkyl, C₂₋₇ alkenyl, C₂₋₇ alkynyl, C₃₋₇ cycloalkyl, or C₄₋₇ cycloalkenyl.

In some embodiments, R⁷ is C₁₋₇ alkyl.

In some embodiments, R⁷ is selected from methyl, ethyl, propyl, isopropyl, butyl, sec-butyl, isobutyl, or tert-butyl.

In some embodiments, R⁷ is C₂₋₇ alkenyl.

In some embodiments, R⁷ is vinyl.

In some embodiments, R⁷ is C₃₋₇ cycloalkyl.

In some embodiments, R⁷ is selected from cyclopropyl, cyclobutyl, cyclopentyl, cyclohexyl or cycloheptyl.

In some embodiments, R⁷ is H.

In some embodiments, the group R⁷ is in position −5 of the piperidine ring.

In some embodiments, the group R⁷ is in position −6 of the piperidine ring.

In some embodiments, the group R⁶ is in position −2 of the piperidine ring.

In some embodiments, the group R⁶ is in position −2 of the piperidine ring and/or the group R⁷ is in position −5 of the piperidine ring.

In some embodiments, the group R⁶ is in position −2 of the piperidine ring and the group R⁷ is in position −5 of the piperidine ring.

In some embodiments, R⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by cycloalkyl, aryl or heteroaryl, wherein the cycloalkyl, aryl or the heteroaryl is optionally substituted by halogen, C₁₋₄ alkyl or C₃₋₅ cycloalkyl

In some embodiments, R⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl.

In some embodiments, R⁴ is selected from C₁₋₇ alkyl, C₃₋₇ cycloalkyl, or C₁₋₃ alkyl substituted by aryl or heteroaryl, wherein the aryl or the heteroaryl is optionally substituted by halogen, C₁₋₄ alkyl or C₃₋₅ cycloalkyl.

In some embodiments, R⁴ is selected from C₂₋₇ alkyl, C₃₋₇ cycloalkyl, or C₁₋₃ alkyl substituted by aryl or heteroaryl, wherein the aryl or the heteroaryl is optionally substituted by halogen, C₁₋₄ alkyl or C₃₋₅ cycloalkyl.

In some embodiments, R⁴ is selected from C₃₋₇ alkyl, C₃₋₇ cycloalkyl, or C₁₋₃ alkyl substituted by aryl or heteroaryl.

In some embodiments, R⁴ is H.

In some embodiments, R⁴ is C₃₋₇ cycloalkyl.

In some embodiments, R⁴ is selected from cyclopropyl, cyclobutyl, cyclopentyl, cyclohexyl or cycloheptyl.

In some embodiments, R⁴ is selected from cyclopropyl or cylcohexyl.

In some embodiments, R⁴ is cyclopropyl.

In some embodiments, R⁴ is cyclohexyl.

In some embodiments, R⁴ is C₁₋₇ alkyl.

In some embodiments, R⁴ is C₂₋₇ alkyl.

In some embodiments, R⁴ is C₃₋₇ alkyl.

In some embodiments, R⁴ is selected from methyl, ethyl, propyl, isopropyl, butyl, sec-butyl, isobutyl, or tert-butyl.

In some embodiments, R⁴ is selected from ethyl, propyl, isopropyl, butyl, sec-butyl, isobutyl, or tert-butyl.

In some embodiments, R⁴ is selected from propyl, isopropyl, butyl, sec-butyl, isobutyl, or tert-butyl.

In some embodiments, R⁴ is isobutyl.

In some embodiments, R⁴ is C₁₋₃ alkyl substituted by cycloalkyl.

In some embodiments, R⁴ is methyl, ethyl, or propyl substituted by cyclopropyl, cyclobutyl, cyclopentyl, cyclohexyl or cycloheptyl.

In some embodiments, R⁴ is ethyl or propyl substituted by cyclopropyl or cyclohexyl.

In some embodiments, R⁴ is C₁₋₃ alkyl substituted by aryl or heteroaryl.

In some embodiments, R⁴ is methyl, ethyl, or propyl substituted by phenyl, imidazole, pyridine, or triazole.

In some embodiments, R⁴ is ethyl or propyl substituted by phenyl or pyridine.

In some embodiments, R⁴ is ethyl substituted by phenyl.

In some embodiments, R⁴ is

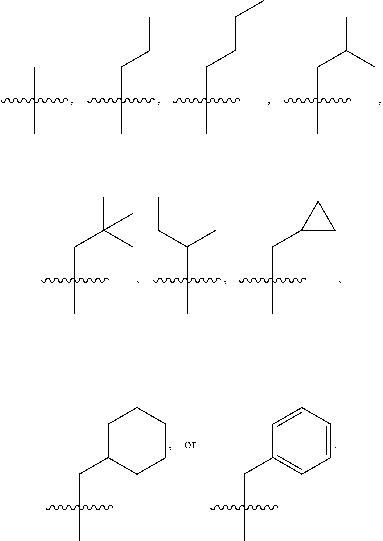

In some embodiments, R⁴ is

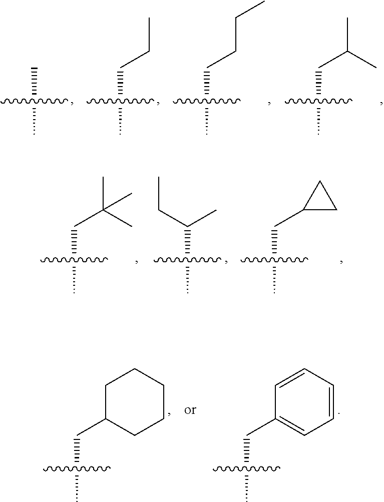

In some embodiments, R⁴ is

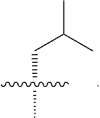

In some embodiments, the compound is of any one of Formulae (IIa), (JIb), or (IIc):

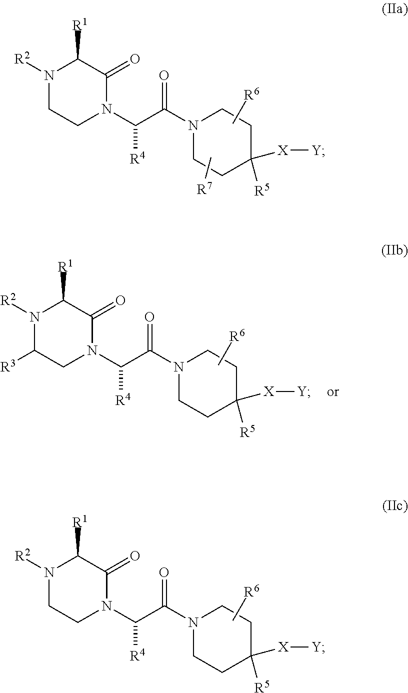

or a pharmaceutically acceptable salt, hydrate, solvate or stereoisomer thereof, wherein R¹, R², R³, R⁴, R⁵, R⁶, R⁷, X, and Y are as described herein.

In some embodiments, the compound is of Formula (IIa) or a pharmaceutically acceptable salt, hydrate, solvate or stereoisomer thereof, wherein R¹, R², R³, R⁴, R⁵, R⁶, R⁷, X, and Y are as described herein.

In some embodiments, the compound is of Formula (IIb) or a pharmaceutically acceptable salt, hydrate, solvate or stereoisomer thereof, wherein R¹, R², R³, R⁴, R⁵, R⁶, R⁷, X, and Y are as described herein.

In some embodiments, the compound is of Formula (IIc) or a pharmaceutically acceptable salt, hydrate, solvate or stereoisomer thereof, wherein R¹, R², R³, R⁴, R⁵, R⁶, R⁷, X, and Y are as described herein.

In some embodiments, the compound is of any one of Formulae (IIIa), (IIIb), (IIIc), or (IIId):

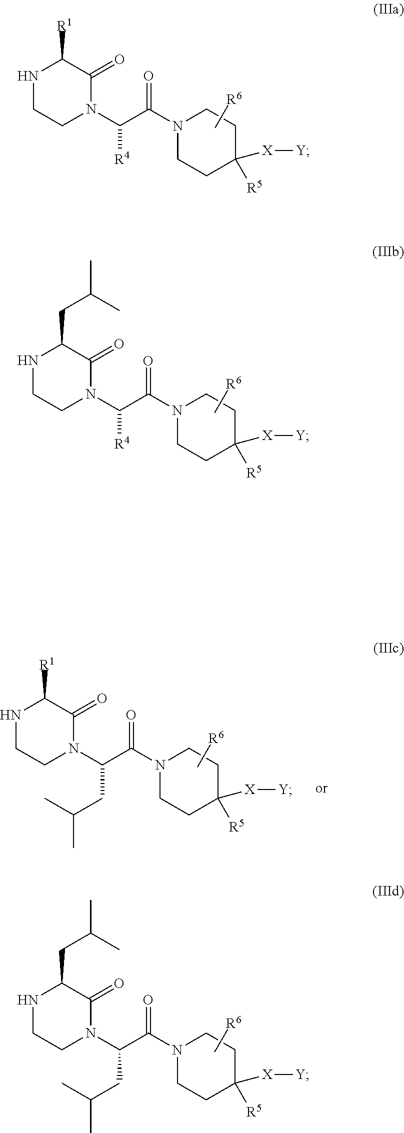

or a pharmaceutically acceptable salt, hydrate, solvate or stereoisomer thereof, wherein R¹, R⁴, R⁵, R⁶, X, and Y are as described herein.

In some embodiments, the compound is of Formula (IIIa) or a pharmaceutically acceptable salt, hydrate, solvate or stereoisomer thereof, wherein R¹, R⁴, R⁵, R⁶, X, and Y are as described herein.

In some embodiments, the compound is of Formula (IIIb) or a pharmaceutically acceptable salt, hydrate, solvate or stereoisomer thereof, wherein R¹, R⁴, R⁵, R⁶, X, and Y are as described herein.

In some embodiments, the compound is of Formula (IIIc) or a pharmaceutically acceptable salt, hydrate, solvate or stereoisomer thereof, wherein R¹, R⁴, R⁵, R⁶, X, and Y are as described herein.

In some embodiments, the compound is of Formula (IIId) or a pharmaceutically acceptable salt, hydrate, solvate or stereoisomer thereof, wherein R¹, R⁴, R⁵, R⁶, X, and Y are as described herein.

In some embodiments, the group R⁶ is in position −2 of the piperidine ring.

In some embodiments, the group R⁶ is in position −3 of the piperidine ring.

In some embodiments, the group R⁶ is in position −2 of the piperidine ring and/or the group R⁷ is in position −5 of the piperidine ring.

In some embodiments, the group R⁶ is in position −2 of the piperidine ring and the group R⁷ is in position −5 of the piperidine ring.

In some embodiments, R⁶ is selected from H, C₁₋₇ alkyl, C₂₋₇ alkenyl, C₂₋₇ alkynyl, C₃₋₇ cycloalkyl, C₄₋₇ cycloakenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; and wherein R⁶ can form a ring with any part of X; or is imidazolidinone.

In some embodiments, R⁶ is selected from H, C₁₋₇ alkyl, C₂₋₇ alkenyl, C₂₋₇ alkynyl, C₃₋₇ cycloalkyl, C₄₋₇ cycloakenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; or is imidazolidinone.

In some embodiments, R⁶ is selected from H, C₁₋₇ alkyl, C₂₋₇ alkenyl, C₂₋₇ alkynyl, C₃₋₇ cycloalkyl, or C₄₋₇ cycloalkenyl; or is imidazolidinone.

In some embodiments, R⁶ is H, C₁₋₇ alkyl, or imidazolidinone.

In some embodiments, R⁶ is H or C₁₋₇ alkyl.

In some embodiments, R⁶ is H.

In some embodiments, R⁶ is in position −2 of the piperidine ring and is H.

In some embodiments, R⁶ is in position −3 of the piperidine ring and is H.

In some embodiments, R⁶ is imidazolidinone.

In some embodiments, R⁶ is

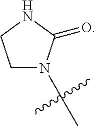 [SMILES: *N1CCNC1=O]

In some embodiments, R⁶ is C₁₋₇ alkyl.

In some embodiments, R⁶ is selected from methyl, ethyl, propyl, isopropyl, butyl, sec-butyl, isobutyl, or tert-butyl.

In some embodiments, R⁶ is methyl.

In some embodiments, R⁶ is in position −2 of the piperidine ring and is C₁₋₇ alkyl.

In some embodiments, R⁶ is in position −2 of the piperidine ring and is selected from methyl, ethyl, propyl, isopropyl, butyl, sec-butyl, isobutyl, or tert-butyl.

In some embodiments, R⁶ is in position −2 of the piperidine ring and is methyl.

In some embodiments, R⁶ is selected from the group consisting of H,

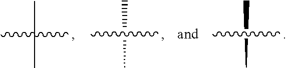

In some embodiments, R⁶ is in position −2 of the piperidine ring and is selected from the group consisting of H,

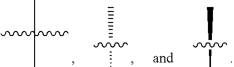

In some embodiments, R⁶ is

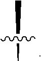

In some embodiments, R⁶ is in position −2 of the piperidine ring and is

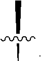

In some embodiments, R⁶ is C₁₋₃ alkyl substituted by C(O)NR⁸R¹¹.

In some embodiments, R⁶ is C₁₋₃ alkyl substituted by C(O)NR⁸R¹¹, wherein R⁸ is H.

In some embodiments, R⁶ is C₁₋₃ alkyl substituted by C(O)NR⁸R¹¹, wherein R¹¹ is H.

In some embodiments, R⁶ is C₁₋₃ alkyl substituted by C(O)NR⁸R¹¹, wherein R⁸ and R¹¹ is H.

In some embodiments, R⁶ is C₁₋₃ alkyl substituted by C(O)N₂.

In some embodiments, R⁶ is methyl, ethyl, or propyl substituted by C(O)N₂.

In some embodiments, R⁶ is ethyl substituted by C(O)N₂.

In some embodiments, R⁶ is propyl substituted by C(O)NH₂.

In some embodiments, R⁶ is selected from the group consisting of

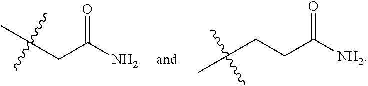 [SMILES: *CC(N)=O ; *CCC(N)=O]

In some embodiments, R⁶ is in position −3 of the piperidine ring and is selected from the group consisting of

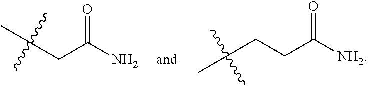 [SMILES: *CC(N)=O ; *CCC(N)=O]

In some embodiments, R⁶ forms a ring with any part of X.

In some embodiments, R⁶ is in position −3 of the piperidine ring and forms a ring with any part of X.

In some embodiments, R⁶ is in position −3 of the piperidine ring and forms a 3-membered, a 4-membered, 5-membered, or 6-membered ring with any part of X.

In some embodiments, R⁶ is in position −3 of the piperidine ring and forms a 4-membered or 6-membered ring with any part of X.

In some embodiments, R⁶ is in position −3 of the piperidine ring and forms a 4-membered or ring with any part of X.

In some embodiments, R⁶ is in position −3 of the piperidine ring and forms a 6-membered ring with any part of X.

In some embodiments, the compound is of any one of Formulae (IVa), (IVb), (IVc) or (IVd):

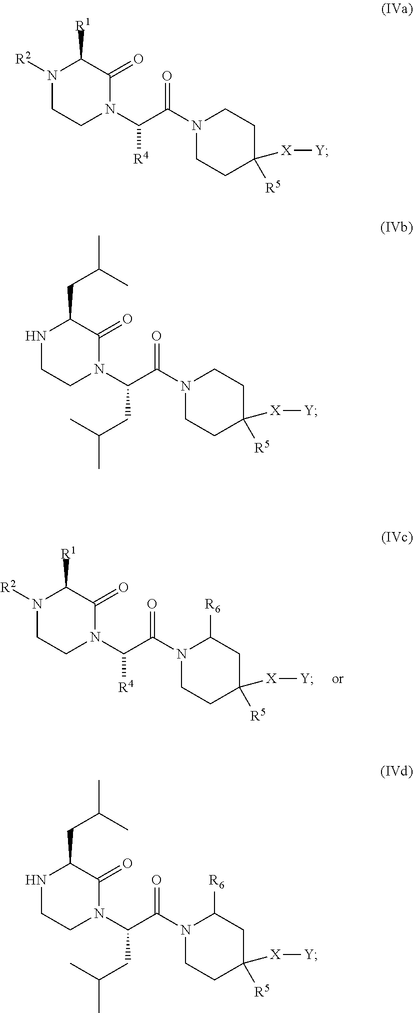

or a pharmaceutically acceptable salt, hydrate, solvate or stereoisomer thereof, wherein R¹, R², R⁴, R⁵, R⁶, X, and Y are as described herein.

In some embodiments, the compound is of Formula (IVa) or a pharmaceutically acceptable salt, hydrate, solvate or stereoisomer thereof, wherein R¹, R², R⁴, R⁵, R⁶, X, and Y are as described herein.

In some embodiments, the compound is of Formula (IVb) or a pharmaceutically acceptable salt, hydrate, solvate or stereoisomer thereof, wherein R¹, R², R⁴, R⁵, R⁶, X, and Y are as described herein.

In some embodiments, the compound is of Formula (IVc) or a pharmaceutically acceptable salt, hydrate, solvate or stereoisomer thereof, wherein R¹, R², R⁴, R⁵, R⁶, X, and Y are as described herein.

In some embodiments, the compound is of Formula (IVd) or a pharmaceutically acceptable salt, hydrate, solvate or stereoisomer thereof, wherein R¹, R², R⁴, R⁵, R⁶, X, and Y are as described herein.

In some embodiments, R⁵ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸; and wherein R⁵ can form a ring with any part of X or Y, wherein the ring optionally contains a carbonyl group;

In some embodiments, R⁵ is selected from H, C₁₋₇ alkyl, OR⁸, or SR⁸; and wherein C₁₋₇ alkyl, OR⁸ or SR⁸ of R⁵ can form a ring with any part of X or Y, wherein the ring optionally contains a carbonyl group.

In some embodiments, R⁵ is selected from H, C₁₋₇ alkyl, OR⁸, or SR⁸; and wherein C₁₋₇ alkyl, OR⁸ or SR⁸ of R⁵ can form a ring with any part of X or, when Y is C(O)NR¹⁰R¹² or NR¹⁰R¹², C₁₋₇ alkyl of R⁵ can form a ring with any part of Y, wherein the ring optionally contains a carbonyl group.

In some embodiments, R⁵ is selected from H, C₁₋₇ alkyl, or OR⁸; and wherein C₁₋₇ alkyl or OR⁸ can form a ring with any part of X or Y, wherein the ring optionally contains a carbonyl group.

In some embodiments, R⁵ is selected from H, C₁₋₇ alkyl, or OR⁸; and wherein C₁₋₇ alkyl or OR⁸ of R⁵ can form a ring with any part of X or, when Y is C(O)NR¹⁰R¹² or NR¹⁰R¹², C₁₋₇ alkyl of R⁵ can form a ring with any part of Y, wherein the ring optionally contains a carbonyl group.

In some embodiments, R⁵ is selected from C₁₋₇ alkyl, OR⁸, or SR⁸; wherein C₁₋₇ alkyl, OR⁸ or SR⁸ can form a ring with any part of X.

In some embodiments, R⁵ is OR⁸, wherein R⁸ of OR⁸ is C₁₋₇ alkyl, and wherein OR⁸ can form a ring with any part of X.

In some embodiments, R⁵ is selected from H and C₁₋₇ alkyl; and wherein C₁₋₇ alkyl can form a ring with any part of X or Y, wherein the ring optionally contains a carbonyl group.

In some embodiments, R⁵ is selected from H and C₁₋₇ alkyl; and wherein C₁₋₇ alkyl of R⁵ can form a ring with any part of X or, when Y is C(O)NR¹⁰R¹² or NR¹⁰R¹², C₁₋₇ alkyl of R⁵ can form a ring with any part of Y, wherein the ring optionally contains a carbonyl group.

In some embodiments, R⁵ is selected from H and C₁₋₇ alkyl.

In some embodiments, R⁵ is selected from H, methyl, and ethyl.

In some embodiments, R⁵ is H.

In some embodiments, R⁵ is methyl.

In some embodiments, R⁵ is ethyl.

In some embodiments, R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl.

In some embodiments, R⁸ and R¹¹ are each independently selected from H, C₁₋₇ alkyl, C₂₋₇ alkenyl, and C₃₋₇ cycloalkyl.

In some embodiments, R⁸ is C₁₋₇ alkyl and/or R¹¹ is selected from H, C₁₋₇ alkyl, C₂₋₇ alkenyl, and C₃₋₇ cycloalkyl.

In some embodiments, R⁸ is C₁₋₇ alkyl and/or R¹¹ is C₁₋₇ alkyl.

In some embodiments, R⁹ is selected from H, halogen, C₁₋₅ alkyl, C₂₋₅ alkenyl, C₂₋₅ alkynyl, C₃₋₅ cycloalkyl, C₁₋₅ alkyl-OR⁸, C₁₋₅ alkyl-SR⁸, C₁₋₅ alkyl-NR⁸R¹¹, C₁₋₅ alkyl-C(O)OR⁸, C₁₋₅ alkyl-C(O)NR⁸R¹¹, C₁₋₅ alkyl-C(O)R¹⁰, CN, C(O)R⁸, C(O)NR⁸R¹¹, C(O)OR⁸, NR⁸C(O)NR⁸R¹¹, OC(O)NR⁸R¹¹, SO₂NR⁸R¹¹, NR⁸SO₂R⁸, OR⁸, NR⁸R¹¹, or S(O)ₙR⁸ wherein n is 0, 1 or 2.

In some embodiments, R⁹ is selected from H, C₁₋₅ alkyl, halogen, C₁₋₅ alkyl-NR⁸R¹¹, C₁₋₅ alkyl-C(O)OR⁸, C₁₋₅ alkyl-C(O)NR⁸R¹¹, CN, C(O)R⁸, C(O)NR⁸R¹¹, C(O)OR⁸, and OR⁸.

In some embodiments, R¹⁰ and R¹² are each independently selected from H, C₁₋₇ alkyl, C₂₋₇ alkenyl, C₂₋₇ alkynyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen, OR⁸, or NR⁸R¹¹.

In some embodiments, R¹⁰ and R¹² are each independently selected from H, C₁₋₇ alkyl, C₂₋₇ alkenyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen or OR⁸.

In some embodiments, R¹⁰ and R¹² are each independently selected from H, C₁₋₇ alkyl, C₃₋₇ cycloalkyl, C₁₋₃ alkanediyl-O—C₁₋₃ alkanediyl-O—C₁₋₃ alkanediyl, or C₁₋₃ alkyl-aryl, all these groups optionally substituted by halogen.

In some embodiments, Y is C(O)NR¹⁰R¹² or NR¹⁰R¹², and R¹⁰ and R¹² can form a ring optionally substituted by R⁹ or R¹⁴; wherein the ring contains the N of NR¹⁰R¹² and optionally one further heteroatom selected from O and N, wherein if the one further heteroatom is N, it is optionally substituted by R⁸.

In some embodiments, R¹³ is C₁₋₅ alkyl substituted by a bicyclic ring optionally containing at least one heteroatom and a carbonyl group.

In some embodiments, R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl.

In some embodiments, R¹⁴ is selected from C₁₋₇ alkyl, C₃₋₇ cycloalkyl, or C₁₋₃ alkyl substituted by aryl or heteroaryl, wherein the aryl or the heteroaryl is optionally substituted by halogen, C₁₋₄ alkyl or C₃₋₅ cycloalkyl.

In some embodiments, R¹⁴ is selected from C₁₋₇ alkyl and C₃₋₇ cycloalkyl.

In some embodiments, R¹⁴ is C₁₋₇ alkyl.

In some embodiments, each R¹⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.

In some embodiments, each R¹⁵ is independently selected from H, C₁₋₇ alkyl, and C₃₋₇ cycloalkyl.

In some embodiments, each R¹⁵ is independently selected from H and C₁₋₇ alkyl.

In some embodiments, X is selected from a bond, C₁₋₇ alkanediyl, C₂₋₇ alkenediyl, C₂₋₇ alkynediyl, C₃₋₉ cycloalkanediyl, C₄₋₆ cycloalkenediyl, —O—, C₁₋₃ alkanediyl-O—, —O—C₁₋₇ alkanediyl, —O—C₃₋₉ cycloalkanediyl, C₁₋₃ alkanediyl-O—C₁₋₇ alkanediyl, C₁₋₇ heteroalkanediyl, or —S—C₁₋₇ alkanediyl; and wherein X can form a ring or a polycyclic system with any part of R⁵, R⁶, or Y, wherein the ring optionally contains a carbonyl group.

In some embodiments, X is selected from a bond, C₁₋₇ alkanediyl, C₂₋₇ alkenediyl, C₂₋₇ alkynediyl, C₃₋₆ cycloalkanediyl, C₄₋₆ cycloalkenediyl, —O—, C₁₋₃ alkanediyl-O—, —O—C₁₋₇ alkanediyl, C₁₋₃ alkanediyl-O—C₁₋₇ alkanediyl, C₁₋₇ heteroalkanediyl, or —S—C₁₋₇ alkanediyl; and wherein X can form a ring with any part of R⁵ or Y, wherein the ring optionally contains a carbonyl group.

In some embodiments, X is selected from a bond, C₁₋₇ alkanediyl, —O—, C₁₋₃ alkanediyl-O—, —O—C₁₋₇ alkanediyl, C₁₋₃ alkanediyl-O—C₁₋₇ alkanediyl, C₁₋₇ heteroalkanediyl, or —S—C₁₋₇ alkanediyl; and wherein X can form a ring with any part of R⁵ or Y, wherein the ring optionally contains a carbonyl group.

In some embodiments, X is selected from a bond, C₁₋₇ alkanediyl, —O—, C₁₋₃ alkanediyl-O—, —O—C₁₋₇ alkanediyl, C₁₋₃ alkanediyl-O—C₁₋₇ alkanediyl, C₁₋₇ heteroalkanediyl, or —S—C₁₋₇ alkanediyl; and wherein X can form a ring with any part of R⁵ or, when Y is C(O)NR¹⁰R¹² or NR¹⁰R¹², X can form a ring with any part of Y, wherein the ring optionally contains a carbonyl group.

In some embodiments, X is selected from a bond, —O—C₁₋₇ alkanediyl, —S—C₁₋₇ alkanediyl and C₁₋₇ alkanediyl; and wherein —O—C₁₋₇ alkanediyl, S—C₁₋₇ alkanediyl or C₁₋₇ alkanediyl of X can form a ring with any part of R⁵ or Y, wherein the ring optionally contains a carbonyl group.

In some embodiments, X is selected from a bond, —O—C₁₋₇ alkanediyl and C₁₋₇ alkanediyl; and wherein —O—C₁₋₇ alkanediyl or C₁₋₇ alkanediyl of X can form a ring with any part of R⁵ or, when Y is C(O)NR¹⁰R¹² or NR¹⁰R¹², C₁₋₇ alkanediyl of X can form a ring with any part of Y, wherein the ring optionally contains a carbonyl group.

In some embodiments, X is selected from a bond, —O—C₁₋₇ alkanediyl, S—C₁₋₇ alkanediyl and C₁₋₇ alkanediyl, and wherein —O—C₁₋₇ alkanediyl, S—C₁₋₇ alkanediyl or C₁₋₇ alkanediyl can form a ring with any part of R⁵, wherein the ring optionally contains a carbonyl group.

In some embodiments, X is selected from a bond and C₁₋₇ alkanediyl, wherein C₁₋₇ alkanediyl can form a ring with any part of R⁵ or Y.

In some embodiments, X is selected from a bond and C₁₋₇ alkanediyl, wherein C₁₋₇ alkanediyl of X can form a ring with any part of R⁵ or, when Y is C(O)NR¹⁰R¹² or NR¹⁰R¹², C₁₋₇ alkanediyl of X can form a ring with any part of Y.

In some embodiments, X is selected from a bond and C₁₋₇ alkanediyl, wherein C₁₋₇ alkanediyl can form a ring with any part of Y.

In some embodiments, X is selected from a bond and C₁₋₇ alkanediyl, wherein C₁₋₇ alkanediyl of X can form a ring with any part of Y when Y is C(O)NR¹⁰R¹² or NR¹⁰R¹².

In some embodiments, the ring which can be formed by R⁵ and any part of X or Y, the ring which can be formed by X and any part of R⁵ or Y, and/or the ring which can be formed by Y and any part of X or R⁵ is a non-aromatic ring, preferably a non-aromatic ring containing between four and six atoms e.g. between four and six carbon and heteroatoms, more preferably a non-aromatic ring containing between three and five carbon and one nitrogen atom or a non-aromatic ring containing between two and four carbon and one or two, preferably two, oxygen or sulfur, preferably oxygen, atoms.

In some embodiments, Y is C(O)NR¹⁰R¹² or NR¹⁰R¹² and R¹⁰ and R¹² can form a ring wherein the ring contains the N of NR¹⁰R¹² and optionally one further heteroatom selected from O and N, wherein if the one further heteroatom is N, it is optionally substituted by R⁸, wherein the ring, R¹⁰ and R¹² can form is a non-aromatic ring, preferably a non-aromatic ring containing between four and seven atoms e.g. between three and six carbon atoms and the N of NR¹⁰R¹², or between three and five carbon atoms and the N of NR¹⁰R¹² and optionally one further heteroatom selected from O and N, wherein if the one further heteroatom is N, it is optionally substituted by R⁸.

In some embodiments, R² is C(O)NR¹⁵R¹⁵ and both R¹⁵ can form a ring wherein the ring contains the N of NR¹⁵R¹⁵ and optionally one further heteroatom selected from O and N, wherein if the one further heteroatom is N, it is optionally substituted by R⁸, wherein the ring, both R⁵ can form is a non-aromatic ring, preferably a non-aromatic ring containing between four and seven atoms e.g. between three and six carbon atoms and the N of NR¹⁵R¹⁵, or between three and five carbon atoms and the N of NR¹⁵R¹⁵ and optionally one further heteroatom selected from O and N, wherein if the one further heteroatom is N, it is optionally substituted by R⁸.

In some embodiments, the integer n of S(O)ₙR⁸ is 1 or 2.

In some embodiments, Y is selected from H, C(O)NR¹⁰R¹², C(O)OR¹⁰, R¹⁰NC(O)NR¹⁰R¹², OC(O)R¹⁰, OC(O)NR¹⁰R¹², S(O)ₙR⁸ wherein n is 0, 1 or 2, SO₂NR¹⁰R¹², NR¹⁰SO₂R¹⁰, NR¹⁰R¹², HNCOR⁸, CN, C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or aryl, heteroaryl wherein the aryl or heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, Y is selected from NR¹⁰R¹² and C₃₋₇-cycloalkyl optionally containing a heteroatom in the ring selected from O and N wherein if the heteroatom is N it is optionally substituted by R⁸; and wherein Y can form a ring with any part of X or R⁵; with the proviso that when Y is NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, R⁵ is selected from H and C₁₋₇ alkyl; wherein C₁₋₇ alkyl of R⁵ can form a ring with any part of Y;
X is selected from a bond and C₁₋₇ alkanediyl, and wherein C₁₋₇ alkanediyl of X can form a ring with any part of Y;
Y is selected from NR¹⁰R¹² and C₃₋₇-cycloalkyl optionally containing a hetero atom in the ring wherein the heteroatom is N and is optionally substituted by R⁸ wherein R⁸ is C₁₋₇ alkyl; wherein Y can form a ring with any part of C₁₋₇ alkanediyl of X or with any part of C₁₋₇ alkyl of R⁵; with the proviso that when Y is NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸; and
R¹⁰ and R¹² are each independently selected from H, C₁₋₇ alkyl, C₃₋₇ cycloalkyl, or C₁₋₃ alkyl-aryl, all these groups optionally substituted by halogen.

In some embodiments, R⁵ is selected from C₁₋₇ alkyl, OR⁸, or SR⁸; wherein C₁₋₇ alkyl, OR⁸ or SR⁸ of R⁵ can form a ring with any part of X;
X is selected from —O—C₁₋₇ alkanediyl, —S—C₁₋₇ alkanediyl, or C₁₋₇ alkanediyl, and wherein —O—C₁₋₇ alkanediyl, —S—C₁₋₇ alkanediyl or C₁₋₇ alkanediyl of X can form a ring with any part of R⁵; and
Y is NR¹⁰R¹², wherein R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, R⁵ is OR⁸, wherein R⁸ of OR⁸ is C₁₋₇ alkyl, wherein OR⁸ of R⁵ can form a ring with any part of X;
X is —O—C₁₋₇ alkanediyl and wherein —O—C₁₋₇ alkanediyl of X can form a ring with any part of R⁵; and
Y is NR¹⁰R¹² wherein R¹⁰ and R¹² can form a ring wherein the ring contains the N of NR¹⁰R¹² and four or five carbon atoms.

In some embodiments, Y is aryl or heteroaryl, wherein the aryl or the heteroaryl is optionally substituted by one or more of R⁸; or S-heteroaryl, wherein the S-heteroaryl is optionally substituted by one or more R¹⁴.

In some embodiments, Y is heteroaryl, wherein the heteroaryl is optionally substituted by one or more of R⁸; or S-heteroaryl, wherein the S-heteroaryl is optionally substituted by one or more R¹⁴.

In some embodiments, R⁵ is selected from H and C₁₋₇ alkyl;
X is selected from a bond and C₁₋₇ alkanediyl; and
Y is aryl or heteroaryl, wherein the aryl or the heteroaryl is optionally substituted by one or more of R⁸; or S-heteroaryl wherein the S-heteroaryl is optionally substituted by one or more R¹⁴.

In some embodiments, R⁵ is selected from H and C₁₋₇ alkyl;
X is selected from a bond and C₁₋₇ alkanediyl; and
Y is heteroaryl, wherein the heteroaryl is optionally substituted by one or more of R⁸; or S-heteroaryl wherein the S-heteroaryl is optionally substituted by one or more R¹⁴.

In some embodiments, R⁵ is selected from H and C₁₋₇ alkyl;
X is selected from a bond and C₁₋₇ alkanediyl; and
Y is aryl or heteroaryl, wherein the aryl or the heteroaryl is optionally substituted by one of R⁸ wherein R⁸ is selected from C₁₋₇ alkyl, C₂₋₇ alkenyl, or C₃₋₇ cycloalkyl; or S-heteroaryl wherein the S-heteroaryl is optionally substituted by one of R⁴ wherein R¹⁴ is selected from C₁₋₇ alkyl, C₂₋₇ alkenyl, or C₃₋₇ cycloalkyl.

In some embodiments, R⁵ is selected from H and C₁₋₇ alkyl;
X is selected from a bond and C₁₋₇ alkanediyl; and
Y is heteroaryl, wherein the heteroaryl is optionally substituted by one of R⁸ wherein R⁸ is selected from C₁₋₇ alkyl, C₂₋₇ alkenyl, C₃₋₇ cycloalkyl; or S-heteroaryl wherein the S-heteroaryl is optionally substituted by one of R⁴ wherein R¹⁴ is selected from C₁₋₇ alkyl, C₂₋₇ alkenyl, C₃₋₇ cycloalkyl.

In some embodiments, the compound is of any one of Formulae (Va), (Vb), (Vc), or (Vd):

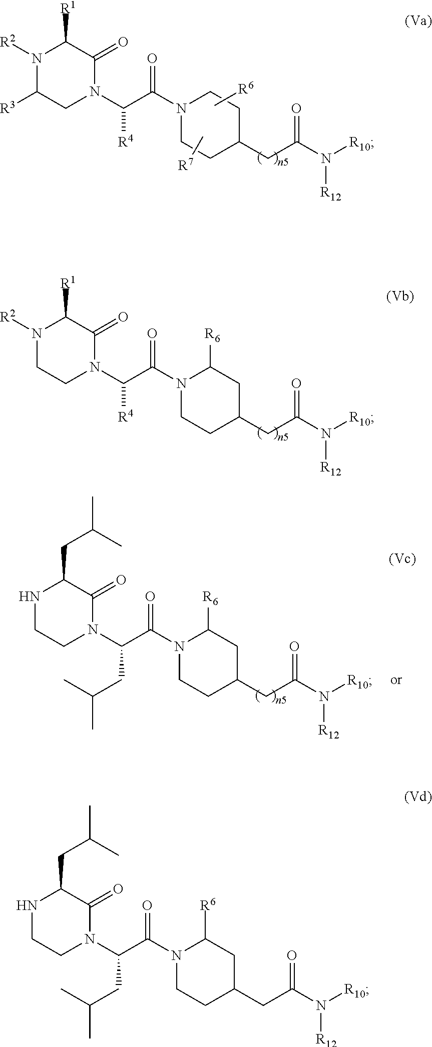

or a pharmaceutically acceptable salt, hydrate, solvate or stereoisomer thereof, wherein n5 is 0, 1, 2, 3, 4, 5, 6, or 7, preferably 1, 2, or 3, and R¹, R², R³, R⁴, R⁶, R⁷, R¹⁰ and R¹² are as described herein.

In some embodiments, the compound is of Formula (Va) or a pharmaceutically acceptable salt, hydrate, solvate or stereoisomer thereof, wherein n5 is 0, 1, 2, 3, 4, 5, 6, or 7, preferably 1, 2, or 3, and R¹, R², R³, R⁴, R⁶, R⁷, R¹⁰ and R¹² are as described herein.

In some embodiments, the compound is of Formula (Vb) or a pharmaceutically acceptable salt, hydrate, solvate or stereoisomer thereof, wherein n5 is 0, 1, 2, 3, 4, 5, 6, or 7, preferably 1, 2, or 3, and R¹, R², R³, R⁴, R⁶, R⁷, R¹⁰ and R¹² are as described herein.

In some embodiments, the compound is of Formula (Vc) or a pharmaceutically acceptable salt, hydrate, solvate or stereoisomer thereof, wherein n5 is 0, 1, 2, 3, 4, 5, 6, or 7, preferably 1, 2, or 3, and R¹, R², R³, R⁴, R⁶, R⁷, R¹⁰ and R¹² are as described herein.

In some embodiments, the compound is of Formula (Vd) or a pharmaceutically acceptable salt, hydrate, solvate or stereoisomer thereof, wherein n5 is 0, 1, 2, 3, 4, 5, 6, or 7, preferably 1, 2, or 3, and R¹, R², R³, R⁴, R⁶, R⁷, R¹⁰ and R¹² are as described herein.

In some embodiments, R⁵ is H and X—Y is

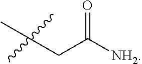 [SMILES: *CC(N)=O]

In some embodiments, the compound is of any one of Formulae (VIa), (VIb), (VIc), or (VId).

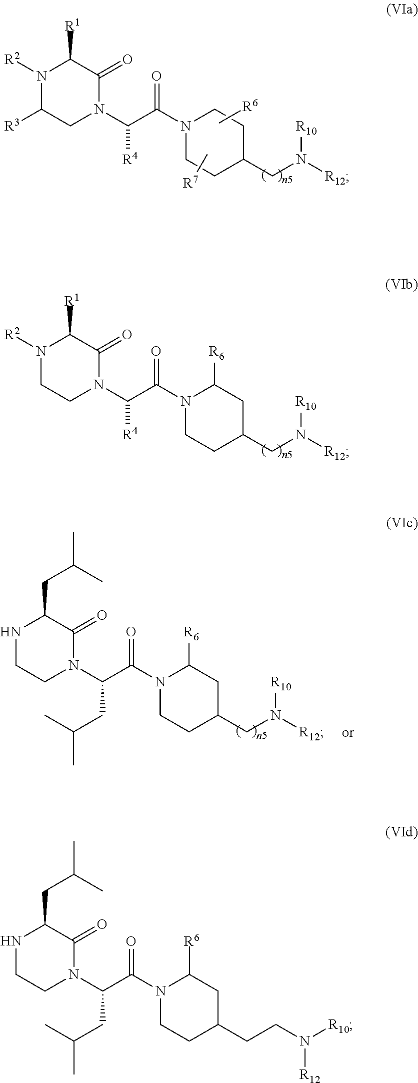

or a pharmaceutically acceptable salt, hydrate, solvate or stereoisomer thereof, wherein n5 is 0, 1, 2, 3, 4, 5, 6, or 7, preferably 1, 2, or 3, and R¹, R², R³, R⁴, R⁶, R⁷, R¹⁰ and R¹² are as described herein.

In some embodiments, the compound is of Formula (VIa) or a pharmaceutically acceptable salt, hydrate, solvate or stereoisomer thereof, wherein n5 is 0, 1, 2, 3, 4, 5, 6, or 7, preferably 1, 2, or 3, and R¹, R², R³, R⁴, R⁶, R⁷, R¹⁰ and R¹² are as described herein.

In some embodiments, the compound is of Formula (VIb) or a pharmaceutically acceptable salt, hydrate, solvate or stereoisomer thereof, wherein n5 is 0, 1, 2, 3, 4, 5, 6, or 7, preferably 1, 2, or 3, and R¹, R², R³, R⁴, R⁶, R⁷, R¹⁰ and R¹² are as described herein.

In some embodiments, the compound is of Formula (VIc) or a pharmaceutically acceptable salt, hydrate, solvate or stereoisomer thereof, wherein n5 is 0, 1, 2, 3, 4, 5, 6, or 7, preferably 1, 2, or 3, and R¹, R², R³, R⁴, R⁶, R⁷, R¹⁰ and R¹² are as described herein.

In some embodiments, the compound is of Formula (VId) or a pharmaceutically acceptable salt, hydrate, solvate or stereoisomer thereof, wherein n5 is 0, 1, 2, 3, 4, 5, 6, or 7, preferably 1, 2, or 3, and R¹, R², R³, R⁴, R⁶, R⁷, R¹⁰ and R¹² are as described herein.

In some embodiments, Y is C(O)NR¹⁰R¹², wherein R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, R⁸ is H and X—Y is

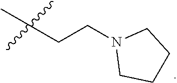 [SMILES: *CCN1CCCC1]

In some embodiments, R⁵ is selected from H and C₁₋₇ alkyl;
X is selected from a bond and C₁₋₇ alkanediyl;
Y is C(O)NR¹⁰R¹², wherein R¹⁰ and R¹² can form a ring wherein the ring contains the N of NR¹⁰R¹² and optionally one further heteroatom selected from O and N, wherein if the one further heteroatom is N, it is optionally substituted by R⁸; and
R¹⁰ and R¹² are each independently selected from H, C₁₋₇ alkyl, C₂₋₇ alkenyl, C₃₋₇ cycloalkyl, or C₁₋₃ alkyl-aryl.

In some embodiments, Y is selected from S-aryl, O-aryl, S-heteroaryl, or O-heteroaryl, wherein the S-aryl, O-aryl, S-heteroaryl, or O-heteroaryl are optionally substituted by one or more R⁹ or R¹⁴.

In some embodiments, Y is selected from O-aryl and O-heteroaryl, wherein the O-aryl and O-heteroaryl are optionally substituted by one or more R⁹ or R¹⁴.

In some embodiments, Y is selected from S-aryl, O-aryl, S-heteroaryl, or O-heteroaryl, wherein the S-aryl, O-aryl, S-heteroaryl, or O-heteroaryl are optionally substituted by one or more R⁹; wherein R⁹ is selected from H, C₁₋₅ alkyl, halogen, C₁₋₅ alkyl-NR⁸R¹¹, C₁₋₅ alkyl-C(O)OR⁸, C₁₋₅ alkyl-C(O)NR⁸R¹¹, CN, C(O)R⁸, C(O)NR⁸R¹¹, C(O)OR¹, and OR⁸.

In some embodiments, R⁵ is selected from H and C₁₋₇ alkyl;
X is selected from a bond and C₁₋₇ alkanediyl; and
Y is selected from O-aryl and O-heteroaryl, wherein the O-aryl and O-heteroaryl is optionally substituted by one or more R⁹; wherein R⁹ is selected from H, C₁₋₅ alkyl, halogen, C₁₋₅ alkyl-NR⁸R¹¹, C₁₋₅ alkyl-C(O)NR⁸R¹¹, C₁₋₅ alkyl-C(O)OR⁸, CN, C(O)R⁸, C(O)NR⁸R¹¹, C(O)OR⁸, and OR⁸.

In some embodiments, Y is C(O)OR¹⁰.

In some embodiments, R⁵ is selected from H and C₁₋₇ alkyl;
X is selected from a bond and C₁₋₇ alkanediyl;
Y is C(O)OR¹⁰; and
R¹⁰ is selected from H, C₁₋₇ alkyl, C₂₋₇ alkenyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by OR⁸.

In some embodiments, Y is H.

In some embodiments, R⁵ is C₁₋₇ alkyl; X is a bond; and Y is H.

In some embodiments, Y is CN.

In some embodiments, R⁵ is H; X is C₁₋₇ alkanediyl; and Y is CN.

In some embodiments, Y is selected from H, C(O)NR¹⁰R¹², C(O)OR¹⁰, NR¹⁰R¹², CN, C₃₋₇-cycloalkyl optionally containing a hetero atom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; aryl, or heteroaryl wherein the aryl or heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, the compound is a compound of Formula (I) or Formula (Ia), wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R² is selected from H, C(O)R¹⁴, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by aryl or heteroaryl, wherein the aryl or the heteroaryl is optionally substituted by halogen, C₁₋₄ alkyl or C₃₋₅ cycloalkyl; with the proviso that when R² is C(O)NR¹⁵R¹⁵, both R¹⁵ can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R⁸ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸; and wherein R⁵ can form a ring with any part of X or Y, wherein the ring optionally contains a carbonyl group;
R⁶ is selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl;
X is selected from a bond, C₁₋₇ alkanediyl, C₂₋₇ alkenediyl, C₂₋₇ alkynediyl, C₃₋₆ cycloalkanediyl, C₄₋₆ cycloalkenediyl, —O—, C₁₋₃ alkanediyl-O—, —O—C₁₋₇ alkanediyl, C₁₋₃ alkanediyl-O—C₁₋₇ alkanediyl, C₁₋₇ heteroalkanediyl, or —S—C₁₋₇ alkanediyl; and wherein X can form a ring with any part of R⁵ or Y, wherein the ring optionally contains a carbonyl group;
Y is selected from H, C(O)NR¹⁰R¹², C(O)OR¹⁰, NR¹⁰R¹², CN, C₃₋₇-cycloalkyl optionally containing a hetero atom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or aryl, heteroaryl wherein the aryl or heteroaryl is optionally substituted by one or more of R⁸; and wherein Y can form a ring with any part of X or R⁵, wherein the ring optionally contains a carbonyl group; with the proviso that when Y is C(O)NR¹⁰R¹¹ or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R⁹ is selected from H, halogen, C₁₋₅ alkyl, C₂₋₅ alkenyl, C₂₋₅ alkynyl, C₃₋₅ cycloalkyl, halogen, C₁₋₅ alkyl-OR⁸, C₁₋₅ alkyl-SR⁸, C₁₋₅ alkyl-NR⁸R¹¹, C₁₋₅ alkyl-C(O)OR⁸, C₁₋₅ alkyl-C(O)NR⁸R¹¹, C₁₋₅ alkyl-C(O)R¹⁰, CN, C(O)R⁸, C(O)NR⁸R¹¹, C(O)OR⁸, NR⁸C(O)NR⁸R¹¹, OC(O)NR⁸R¹¹, SO₂NR⁸R¹¹, NR⁸SO₂R⁸, OR⁸, NR⁸R¹¹, or S(O)ₙR⁸ wherein n is 0, 1 or 2;
R¹⁰ and R¹² are each independently selected from H, C₁₋₇ alkyl, C₂₋₇ alkenyl, C₂₋₇ alkynyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen, OR⁸, or NR⁸R¹¹;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl; each R¹⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.

In some embodiments, Y is selected from H, C(O)NR¹⁰R¹², C(O)OR¹⁰, NR¹⁰R¹², CN, C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or aryl, heteroaryl wherein the aryl or heteroaryl is optionally substituted by one or more of R⁸; and wherein, when Y is C(O)NR¹⁰R¹² or NR¹⁰R¹², Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, the compound is a compound of Formula (I) or Formula (Ia), wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R² is selected from H, C(O)R¹⁴, C(O)NR¹⁵R¹⁵, C(O)OR⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by aryl or heteroaryl, wherein the aryl or the heteroaryl is optionally substituted by halogen, C₁₋₄ alkyl or C₃₋₅ cycloalkyl; with the proviso that when R² is C(O)NR¹⁵R¹⁵, both R⁵ can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R⁵ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸; and wherein R⁵ can form a ring with any part of X or, when Y is C(O)NR¹⁰R¹² or NR¹⁰R¹², R⁵ can form a ring with any part of Y, wherein the ring optionally contains a carbonyl group;
R⁶ is selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl;
X is selected from a bond, C₁₋₇ alkanediyl, C₂₋₇ alkenediyl, C₂₋₇ alkynediyl, C₃₋₆ cycloalkanediyl, C₄₋₆ cycloalkenediyl, —O—, C₁₋₃ alkanediyl-O—, —O—C₁₋₇ alkanediyl, C₁₋₃ alkanediyl-O—C₁₋₇ alkanediyl, C₁₋₇ heteroalkanediyl, or —S—C₁₋₇ alkanediyl; and wherein X can form a ring with any part of R⁵ or, when Y is C(O)NR¹⁰R¹² or NR¹⁰R¹², X can form a ring with any part of Y, wherein the ring optionally contains a carbonyl group;
Y is selected from H, C(O)NR¹⁰R¹², C(O)OR¹⁰, NR¹⁰R¹², CN, C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or aryl, heteroaryl wherein the aryl or heteroaryl is optionally substituted by one or more of R⁸; and wherein, when Y is C(O)NR¹⁰R¹² or NR¹⁰R¹², Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R⁹ is selected from H, halogen, C₁₋₅ alkyl, C₂₋₅ alkenyl, C₂₋₅ alkynyl, C₃₋₅ cycloalkyl, C₁₋₅ alkyl-OR⁸, C₁₋₅ alkyl-SR⁸, C₁₋₅ alkyl-NR⁸R¹¹, C₁₋₅ alkyl-C(O)OR⁸, C₁₋₅ alkyl-C(O)NR⁸R¹¹, C₁₋₅ alkyl-C(O)R¹⁰, CN, C(O)R⁸, C(O)NR⁸R¹¹, C(O)OR⁸, NR⁸C(O)NR⁸R¹¹, OC(O)NR⁸R¹¹, SO₂NR⁸R¹¹, NR⁸SO₂R⁸, OR⁸, NR⁸R¹¹, or S(O)ₙR⁸ wherein n is 0, 1 or 2;
R¹⁰ and R¹² are each independently selected from H, C₁₋₇ alkyl, C₂₋₇ alkenyl, C₂₋₇ alkynyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen, OR⁸, or NR⁸R¹¹;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
each R⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.

In some embodiments, the compound is a compound of Formula (I) or Formula (Ia), wherein:
R¹ is selected from C₃₋₇ alkyl, C₃₋₇ cycloalkyl, or C₁₋₃ alkyl substituted by aryl or heteroaryl;
R² is selected from H, C(O)R¹⁴, C₁₋₇ alkyl, C₃₋₇ cycloalkyl, C₁₋₅ alkyl-OR⁸; C₁₋₅ alkyl-NHCOR¹³ wherein R¹³ is pentylamino-5-oxopentyl-7-thia-2,4-diazabicyclo[3.3.0]octan-3-one; or C₁₋₃ alkyl substituted by aryl, wherein the aryl is optionally substituted by halogen, C₁₋₄ alkyl or C₃₋₅ cycloalkyl;
R³ and R⁷ are H;
R⁴ is selected from C₃₋₇ alkyl, C₃₋₇ cycloalkyl, or C₁₋₃ alkyl substituted by aryl or heteroaryl;
R⁵ is selected from H, C₁₋₇ alkyl, or OR⁸; and wherein C₁₋₇ alkyl or OR⁸ of R⁵ can form a ring with any part of X or, when Y is C(O)NR¹⁰R¹² or NR¹⁰R¹², C₁₋₇ alkyl of R⁵ can form a ring with any part of Y, wherein the ring optionally contains a carbonyl group;
R⁶ is H, C₁₋₇ alkyl, or imidazolidinone;
R⁸ and R¹¹ are each independently selected from H, C₁₋₇ alkyl, C₂₋₇ alkenyl, and C₃₋₇ cycloalkyl;
X is selected from a bond, —O—C₁₋₇ alkanediyl and C₁₋₇ alkanediyl; and wherein —O—C₁₋₇ alkanediyl or C₁₋₇ alkanediyl of X can form a ring with any part of R⁵ or, when Y is C(O)NR¹⁰R¹² or NR¹⁰R¹², C₁₋₇ alkanediyl of X can form a ring with any part of Y, wherein the ring optionally contains a carbonyl group;
Y is selected from H, C(O)NR¹⁰R¹², C(O)OR¹⁰, NR¹⁰R¹², CN, C₃₋₇-cycloalkyl optionally containing a heteroatom in the ring selected from O and N wherein if the heteroatom is N it is optionally substituted by R⁸; O-aryl, S-heteroaryl, O-heteroaryl wherein the O-aryl or the O-heteroaryl are optionally substituted by one or more R⁹ and wherein the S-heteroaryl is optionally substituted by one or more R¹⁴; or aryl, heteroaryl wherein the aryl or the heteroaryl is optionally substituted by one or more of R⁸; and wherein, when Y is C(O)NR¹⁰R¹² or NR¹⁰R¹², Y can form a ring with any part of C₁₋₇ alkanediyl of X or any part of C₁₋₇ alkyl of R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R⁹ is selected from H, C₁₋₅ alkyl, halogen, C₁₋₅ alkyl-NR⁸R¹¹, C₁₋₅ alkyl-C(O)OR⁸, C₁₋₅ alkyl-C(O)NR⁸R¹¹, CN, C(O)R⁸, C(O)NR⁸R¹¹, C(O)OR⁸, and OR⁸;
R¹⁰ and R¹² are each independently selected from H, C₁₋₇ alkyl, C₂₋₇ alkenyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen or OR⁸; and
R¹⁴ is C₁₋₇ alkyl.

In some embodiments, Y is selected from H, C(O)NR¹⁰R¹², C(O)OR¹⁰, NR¹⁰R¹², C₃₋₇-cycloalkyl optionally containing a hetero atom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or aryl, heteroaryl wherein the aryl or heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, Y is selected from H, C(O)NR¹⁰R¹², C(O)OR¹⁰, NR¹⁰R¹², C₃₋₇-cycloalkyl optionally containing a hetero atom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or aryl, heteroaryl wherein the aryl or heteroaryl is optionally substituted by one or more of R⁸; and wherein, when Y is C(O)NR¹⁰R¹² or NR¹⁰R¹², Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, the compound is a compound of Formula (I) or Formula (Ia), wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R² is selected from H, C(O)R¹⁴, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by aryl or heteroaryl, wherein the aryl or the heteroaryl is optionally substituted by halogen, C₁₋₄ alkyl or C₃₋₅ cycloalkyl; with the proviso that when R² is C(O)NR¹⁵R¹⁵, both R¹⁵ can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R⁸ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸; and wherein R⁵ can form a ring with any part of X or Y, wherein the ring optionally contains a carbonyl group;
R⁶ is selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl;
X is selected from a bond, C₁₋₇ alkanediyl, C₂₋₇ alkenediyl, C₂₋₇ alkynediyl, C₃₋₆ cycloalkanediyl, C₄₋₆ cycloalkenediyl, —O—, C₁₋₃ alkanediyl-O—, —O—C₁₋₇ alkanediyl, C₁₋₃ alkanediyl-O—C₁₋₇ alkanediyl, C₁₋₇ heteroalkanediyl, or —S—C₁₋₇ alkanediyl; and wherein X can form a ring with any part of R⁵ or Y, wherein the ring optionally contains a carbonyl group;
Y is selected from H, C(O)NR¹⁰R¹², C(O)OR¹⁰, NR¹⁰R¹², C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or aryl, heteroaryl wherein the aryl or heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R⁹ is selected from H, halogen, C₁₋₅ alkyl, C₂₋₅ alkenyl, C₂₋₅ alkynyl, C₃₋₅ cycloalkyl, halogen, C₁₋₅ alkyl-OR⁸, C₁₋₅ alkyl-SR⁸, C₁₋₅ alkyl-NR⁸R¹¹, C₁₋₅ alkyl-C(O)NR⁸R¹¹, C₁₋₅ alkyl-C(O)OR⁸, C₁₋₅ alkyl-C(O)R¹⁰, CN, C(O)R⁸, C(O)NR⁸R¹¹, C(O)OR⁸, NR⁸C(O)NR⁸R¹¹, OC(O)NR⁸R¹¹, SO₂NR⁸R¹¹, NR⁸SO₂R⁸, OR⁸, NR⁸R¹¹, or S(O)ₙR⁸ wherein n is 0, 1 or 2;
R¹⁰ and R¹² are each independently selected from H, C₁₋₇ alkyl, C₂₋₇ alkenyl, C₂₋₇ alkynyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen, OR⁸, or NR⁸R¹¹;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl; and
each R¹⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.

In some embodiments, the compound is a compound of Formula (I) or Formula (Ia), wherein:
R¹ is selected from C₃₋₇ alkyl, C₃₋₇ cycloalkyl, or C₁₋₃ alkyl substituted by aryl or heteroaryl;
R² is selected from H, C(O)R¹⁴, C₁₋₇ alkyl, C₃₋₇ cycloalkyl, C₁₋₅ alkyl-OR⁸; C₁₋₅ alkyl-NHCOR¹³ wherein R¹³ is pentylamino-5-oxopentyl-7-thia-2,4-diazabicyclo[3.3.0]octan-3-one; or C₁₋₃ alkyl substituted by aryl, wherein the aryl is optionally substituted by halogen, C₁₋₄ alkyl or C₃₋₅ cycloalkyl;
R³ and R⁷ are H;
R⁴ is selected from C₃₋₇ alkyl, C₃₋₇ cycloalkyl, or C₁₋₃ alkyl substituted by aryl or heteroaryl;
R⁸ is selected from H, C₁₋₇ alkyl, or OR⁸; and wherein C₁₋₇ alkyl or OR⁸ of R⁵ can form a ring with any part of X or, when Y is C(O)NR¹⁰R¹¹ or NR¹⁰R¹¹, C₁₋₇ alkyl of R⁵ can form a ring with any part of Y, wherein the ring optionally contains a carbonyl group;
R⁶ is H, C₁₋₇ alkyl, or imidazolidinone;
R⁸ and R¹¹ are each independently selected from H, C₁₋₇ alkyl, C₂₋₇ alkenyl, and C₃₋₇ cycloalkyl;
X is selected from a bond, —O—C₁₋₇ alkanediyl and C₁₋₇ alkanediyl; and wherein —O—C₁₋₇ alkanediyl or C₁₋₇ alkanediyl of X can form a ring with any part of R⁸ or, when Y is C(O)NR¹⁰R¹² or NR¹⁰R¹², C₁₋₇ alkanediyl of X can form a ring with any part of Y, wherein the ring optionally contains a carbonyl group;
Y is selected from H, C(O)NR¹⁰R¹², C(O)OR¹⁰, NR¹⁰R¹², C₃₋₇-cycloalkyl optionally containing a heteroatom in the ring selected from O and N wherein if the heteroatom is N it is optionally substituted by R⁸; O-aryl, S-heteroaryl, O-heteroaryl wherein the O-aryl or the O-heteroaryl are optionally substituted by one or more R⁹ and wherein the S-heteroaryl is optionally substituted by one or more R¹⁴; or aryl, heteroaryl wherein the aryl or the heteroaryl is optionally substituted by one or more of R⁸; and wherein, when Y is C(O)NR¹⁰R¹² or NR¹⁰R¹², Y can form a ring with any part of C₁₋₇ alkanediyl of X or any part of C₁₋₇ alkyl of R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R⁹ is selected from H, C₁₋₅ alkyl, halogen, C₁₋₅ alkyl-NR⁸R¹¹, C₁₋₅ alkyl-C(O)NR⁸R¹¹, C₁₋₅ alkyl-C(O)OR⁸, CN, C(O)R⁸, C(O)NR⁸R¹¹, C(O)OR⁸, and OR⁸;
R¹⁰ and R¹² are each independently selected from H, C₁₋₇ alkyl, C₂₋₇ alkenyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen or OR⁸; and
R¹⁴ is C₁₋₇ alkyl.

In some embodiments, Y is selected from C(O)NR¹⁰R¹², C(O)OR¹⁰, NR¹⁰R¹², C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or aryl, heteroaryl wherein the aryl or heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, Y is selected from C(O)NR¹⁰R¹², C(O)OR¹⁰, NR¹⁰R¹², C₃₋₇-cycloalkyl optionally containing a hetero atom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or aryl, heteroaryl wherein the aryl or heteroaryl is optionally substituted by one or more of R⁸; and wherein, when Y is C(O)NR¹⁰R¹² or NR¹⁰R¹², Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, the compound is a compound of Formula (I) or Formula (Ia), wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R² is selected from H, C(O)R¹⁴, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by aryl or heteroaryl, wherein the aryl or the heteroaryl is optionally substituted by halogen, C₁₋₄ alkyl or C₃₋₅ cycloalkyl; with the proviso that when R² is C(O)NR¹⁵R¹⁵, both R¹⁵ can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R⁸ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸; and wherein R⁵ can form a ring with any part of X or Y, wherein the ring optionally contains a carbonyl group;
R⁶ is selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl;
X is selected from a bond, C₁₋₇ alkanediyl, C₂₋₇ alkenediyl, C₂₋₇ alkynediyl, C₃₋₆ cycloalkanediyl, C₄₋₆ cycloalkenediyl, —O—, C₁₋₃ alkanediyl-O—, —O—C₁₋₇ alkanediyl, C₁₋₃ alkanediyl-O—C₁₋₇ alkanediyl, C₁₋₇ heteroalkanediyl, or —S—C₁₋₇ alkanediyl; and wherein X can form a ring with any part of R⁵ or Y, wherein the ring optionally contains a carbonyl group;
Y is selected from C(O)NR¹⁰R¹², C(O)OR¹⁰, NR¹⁰R¹², C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or aryl, heteroaryl wherein the aryl or heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R⁹ is selected from H, halogen, C₁₋₅ alkyl, C₂₋₅ alkenyl, C₂₋₅ alkynyl, C₃₋₅ cycloalkyl, C₁₋₅ alkyl-OR⁸, C₁₋₅ alkyl-SR⁸, C₁₋₅ alkyl-NR⁸R¹¹, C₁₋₅ alkyl-C(O)OR⁸, C₁₋₅ alkyl-C(O)NR⁸R¹¹, C₁₋₅ alkyl-C(O)R¹⁰, CN, C(O)R⁸, C(O)NR⁸R¹¹, C(O)OR⁸, NR⁸C(O)NR⁸R¹¹, OC(O)NR⁸R¹¹, SO₂NR⁸R¹¹, NR⁸SO₂R⁸, OR⁸, NR⁸R¹¹, or S(O)ₙR⁸ wherein n is 0, 1 or 2;
R¹⁰ and R¹² are each independently selected from H, C₁₋₇ alkyl, C₂₋₇ alkenyl, C₂₋₇ alkynyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen, OR⁸, or NR⁸R¹¹;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl; and
each R⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.

In some embodiments, the compound is a compound of Formula (I) or Formula (Ia), wherein:
R¹ is selected from C₃₋₇ alkyl, C₃₋₇ cycloalkyl, or C₁₋₃ alkyl substituted by aryl or heteroaryl;
R² is selected from H, C(O)R¹⁴, C₁₋₇ alkyl, C₃₋₇ cycloalkyl, C₁₋₅ alkyl-OR⁸; C₁₋₅ alkyl-NHCOR¹³ wherein R¹³ is pentylamino-5-oxopentyl-7-thia-2,4-diazabicyclo[3.3.0]octan-3-one; or C₁₋₃ alkyl substituted by aryl, wherein the aryl is optionally substituted by halogen, C₁₋₄ alkyl or C₃₋₅ cycloalkyl;
R³ and R⁷ are H;
R⁴ is selected from C₃₋₇ alkyl, C₃₋₇ cycloalkyl, or C₁₋₃ alkyl substituted by aryl or heteroaryl;
R⁵ is selected from H, C₁₋₇ alkyl, or OR⁸; and wherein C₁₋₇ alkyl or OR⁸ of R⁵ can form a ring with any part of X or, when Y is C(O)NR¹⁰R¹² or NR¹⁰R¹², C₁₋₇ alkyl of R⁵ can form a ring with any part of Y, wherein the ring optionally contains a carbonyl group;
R⁶ is H or C₁₋₇ alkyl;
R⁸ and R¹¹ are each independently selected from H, C₁₋₇ alkyl, C₂₋₇ alkenyl, and C₃₋₇ cycloalkyl;
X is selected from a bond, —O—C₁₋₇ alkanediyl and C₁₋₇ alkanediyl; and wherein —O—C₁₋₇ alkanediyl or C₁₋₇ alkanediyl of X can form a ring with any part of R⁵ or, when Y is C(O)NR¹⁰R¹² or NR¹⁰R¹², C₁₋₇ alkanediyl of X can form a ring with any part of Y, wherein the ring optionally contains a carbonyl group;
Y is selected from C(O)NR¹⁰R¹², C(O)OR¹⁰, NR¹⁰R¹², C₃₋₇-cycloalkyl optionally containing a heteroatom in the ring selected from O and N wherein if the heteroatom is N it is optionally substituted by R⁸; O-aryl, S-heteroaryl, O-heteroaryl wherein the O-aryl or the O-heteroaryl are optionally substituted by one or more R⁹ and wherein the S-heteroaryl is optionally substituted by one or more R¹⁴; or aryl, heteroaryl wherein the aryl or the heteroaryl is optionally substituted by one or more of R⁸; and wherein, when Y is C(O)NR¹⁰R¹² or NR¹⁰R¹², Y can form a ring with any part of C₁₋₇ alkanediyl of X or any part of C₁₋₇ alkyl of R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R⁹ is selected from H, C₁₋₅ alkyl, halogen, C₁₋₅ alkyl-NR⁸R¹¹, C₁₋₅ alkyl-C(O)OR⁸, C₁₋₅ alkyl-C(O)NR⁸R¹¹, CN, C(O)R⁸, C(O)NR⁸R¹¹, C(O)OR⁸, and OR⁸;
R¹⁰ and R¹² are each independently selected from H, C₁₋₇ alkyl, C₂₋₇ alkenyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen or OR⁸; and
R¹⁴ is C₁₋₇ alkyl.

In some embodiments, Y is selected from C(O)NR¹⁰R¹², C(O)OR¹⁰, NR¹⁰R¹², C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or heteroaryl wherein the heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, Y is selected from C(O)NR¹⁰R¹², C(O)OR¹⁰, NR¹⁰R¹², C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or heteroaryl wherein the heteroaryl is optionally substituted by one or more of R⁸; and wherein, when Y is C(O)NR¹⁰R¹² or NR¹⁰R¹², Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, the compound is a compound of Formula (I) or Formula (Ia), wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R² is selected from H, C(O)R¹⁴, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by aryl or heteroaryl, wherein the aryl or the heteroaryl is optionally substituted by halogen, C₁₋₄ alkyl or C₃₋₅ cycloalkyl; with the proviso that when R² is C(O)NR¹⁵R¹⁵, both R¹⁵ can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R⁵ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸; and wherein R⁵ can form a ring with any part of X or Y, wherein the ring optionally contains a carbonyl group;
R⁶ is selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl;
X is selected from a bond, C₁₋₇ alkanediyl, C₂₋₇ alkenediyl, C₂₋₇ alkynediyl, C₃₋₆ cycloalkanediyl, C₄₋₆ cycloalkenediyl, —O—, C₁₋₃ alkanediyl-O—, —O—C₁₋₇ alkanediyl, C₁₋₃ alkanediyl-O—C₁₋₇ alkanediyl, C₁₋₇ heteroalkanediyl, or —S—C₁₋₇ alkanediyl; and wherein X can form a ring with any part of R⁵ or Y, wherein the ring optionally contains a carbonyl group;
Y is selected from C(O)NR¹⁰R¹², C(O)OR¹⁰, NR¹⁰R¹², C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or heteroaryl wherein the heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R⁹ is selected from H, halogen, C₁₋₅ alkyl, C₂₋₅ alkenyl, C₂₋₅ alkynyl, C₃₋₅ cycloalkyl, C₁₋₅ alkyl-OR⁸, C₁₋₅ alkyl-SR⁸, C₁₋₅ alkyl-NR⁸R¹¹, C₁₋₅ alkyl-C(O)NR⁸R¹¹, C₁₋₅ alkyl-C(O)OR⁸, C₁₋₅ alkyl-C(O)R¹⁰, CN, C(O)R⁸, C(O)NR⁸R¹¹, C(O)OR⁸, NR⁸C(O)NR⁸R¹¹, OC(O)NR⁸R¹¹, SO₂NR⁸R¹¹, NR⁸SO₂R⁸, OR⁸, NR⁸R¹¹, or S(O)ₙR⁸ wherein n is 0, 1 or 2;
wherein R¹⁰ and R¹² are each independently selected from H, C₁₋₇ alkyl, C₂₋₇ alkenyl, C₂₋₇ alkynyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen, OR⁸, or NR⁸R¹¹;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl; and
each R⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.

In some embodiments, the compound is a compound of Formula (I) or Formula (Ia), wherein:
R¹ is selected from C₃₋₇ alkyl, C₃₋₇ cycloalkyl, or C₁₋₃ alkyl substituted by aryl or heteroaryl;
R² is selected from H, C(O)R¹⁴, C₁₋₇ alkyl, C₃₋₇ cycloalkyl, C₁₋₅ alkyl-OR⁸; C₁₋₅ alkyl-NHCOR¹³ wherein R¹³ is pentylamino-5-oxopentyl-7-thia-2,4-diazabicyclo[3.3.0]octan-3-one; or C₁₋₃ alkyl substituted by aryl, wherein the aryl is optionally substituted by halogen, C₁₋₄ alkyl or C₃₋₅ cycloalkyl;
R³ and R⁷ are H;
R⁴ is selected from C₃₋₇ alkyl, C₃₋₇ cycloalkyl, or C₁₋₃ alkyl substituted by aryl or heteroaryl;
R⁸ is selected from H, C₁₋₇ alkyl, or OR⁸; and wherein C₁₋₇ alkyl or OR⁸ of R⁵ can form a ring with any part of X or, when Y is C(O)NR¹⁰R¹² or NR¹⁰R¹², C₁₋₇ alkyl of R⁵ can form a ring with any part of Y, wherein the ring optionally contains a carbonyl group;
R⁶ is H or C₁₋₇ alkyl;
R⁸ and R¹¹ are each independently selected from H, C₁₋₇ alkyl, C₂₋₇ alkenyl, and C₃₋₇ cycloalkyl;
X is selected from a bond, —O—C₁₋₇ alkanediyl and C₁₋₇ alkanediyl; and wherein —O—C₁₋₇ alkanediyl or C₁₋₇ alkanediyl of X can form a ring with any part of R⁸ or, when Y is C(O)NR¹⁰R¹² or NR¹⁰R¹², C₁₋₇ alkanediyl of X can form a ring with any part of Y, wherein the ring optionally contains a carbonyl group;
Y is selected from C(O)NR¹⁰R¹², C(O)OR¹⁰, NR¹⁰R¹², C₃₋₇-cycloalkyl optionally containing a heteroatom in the ring selected from O and N wherein if the heteroatom is N it is optionally substituted by R⁸; O-aryl, S-heteroaryl, O-heteroaryl wherein the O-aryl or the O-heteroaryl are optionally substituted by one or more R⁹ and wherein the S-heteroaryl is optionally substituted by one or more R¹⁴; or heteroaryl wherein the heteroaryl is optionally substituted by one or more of R⁸; and wherein, when Y is C(O)NR¹⁰R¹² or NR¹⁰R¹², Y can form a ring with any part of C₁₋₇ alkanediyl of X or any part of C₁₋₇ alkyl of R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R⁹ is selected from H, C₁₋₅ alkyl, halogen, C₁₋₅ alkyl-NR⁸R¹¹, C₁₋₅ alkyl-C(O)NR⁸R¹¹, C₁₋₅ alkyl-C(O)OR⁸, CN, C(O)R⁸, C(O)NR⁸R¹¹, C(O)OR⁸, and OR⁸;
R¹⁰ and R¹² are each independently selected from H, C₁₋₇ alkyl, C₂₋₇ alkenyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen or OR⁸; and
R¹⁴ is C₁₋₇ alkyl.

In some embodiments, Y is selected from C(O)NR¹⁰R¹², NR¹⁰R¹², C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or heteroaryl wherein the heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, Y is selected from C(O)NR¹⁰R¹², NR¹⁰R¹², C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or heteroaryl wherein the heteroaryl is optionally substituted by one or more of R⁸; and wherein, when Y is C(O)NR¹⁰R¹² or NR¹⁰R¹², Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, the compound is a compound of Formula (I) or Formula (Ia), wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R² is selected from H, C(O)R¹⁴, C(O)NR¹⁵R¹⁵, C(O)OR⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by aryl or heteroaryl, wherein the aryl or the heteroaryl is optionally substituted by halogen, C₁₋₄ alkyl or C₃₋₅ cycloalkyl; with the proviso that when R² is C(O)NR¹⁵R¹⁵, both R¹⁵ can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R⁵ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸; and wherein R⁵ can form a ring with any part of X or Y, wherein the ring optionally contains a carbonyl group;
R⁶ is selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, C₄₋₇ cycloalkenyl;
X is selected from a bond, C₁₋₇ alkanediyl, C₂₋₇ alkenediyl, C₂₋₇ alkynediyl, C₃₋₆ cycloalkanediyl, C₄₋₆ cycloalkenediyl, —O—, C₁₋₃ alkanediyl-O—, —O—C₁₋₇ alkanediyl, C₁₋₃ alkanediyl-O—C₁₋₇ alkanediyl, C₁₋₇ heteroalkanediyl, or —S—C₁₋₇ alkanediyl; and wherein X can form a ring with any part of R⁵ or Y, wherein the ring optionally contains a carbonyl group;
Y is selected from C(O)NR¹⁰R¹², NR¹⁰R¹², C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or heteroaryl wherein the heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R⁹ is selected from H, halogen, C₁₋₅ alkyl, C₂₋₅ alkenyl, C₂₋₅ alkynyl, C₃₋₅ cycloalkyl, C₁₋₅ alkyl-OR⁸, C₁₋₅ alkyl-SR⁸, C₁₋₅ alkyl-NR⁸R¹¹, C₁₋₅ alkyl-C(O)OR⁸, C₁₋₅ alkyl-C(O)NR⁸R¹¹, C₁₋₅ alkyl-C(O)R¹⁰, CN, C(O)R⁸, C(O)NR⁸R¹¹, C(O)OR⁸, NR⁸C(O)NR⁸R¹¹, OC(O)NR⁸R¹¹, SO₂NR⁸R¹¹, NR⁸SO₂R⁸, OR⁸, NR⁸R¹¹, or S(O)ₙR⁸ wherein n is 0, 1 or 2;
R¹⁰ and R¹² are each independently selected from H, C₁₋₇ alkyl, C₂₋₇ alkenyl, C₂₋₇ alkynyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen, OR⁸, or NR⁸R¹¹;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl; and
each R¹⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸.

In some embodiments, the compound is a compound of Formula (I) or Formula (Ia), wherein:
R¹ is selected from C₃₋₇ alkyl, C₃₋₇ cycloalkyl, C₁₋₃ alkyl substituted by aryl or heteroaryl;
R² is selected from H, C(O)R¹⁴, C₁₋₇ alkyl, C₃₋₇ cycloalkyl, C₁₋₅ alkyl-OR⁸; C₁₋₅ alkyl-NHCOR¹³ wherein R¹³ is pentylamino-5-oxopentyl-7-thia-2,4-diazabicyclo[3.3.0]octan-3-one; or C₁₋₃ alkyl substituted by aryl, wherein the aryl is optionally substituted by halogen, C₁₋₄ alkyl or C₃₋₅ cycloalkyl;
R³ and R⁷ are H;
R⁴ is selected from C₃₋₇ alkyl, C₃₋₇ cycloalkyl, or C₁₋₃ alkyl substituted by aryl or heteroaryl;
R⁵ is selected from H, C₁₋₇ alkyl, or OR⁸; and wherein C₁₋₇ alkyl or OR⁸ of R⁵ can form a ring with any part of X or, when Y is C(O)NR¹⁰R¹² or NR¹⁰R¹², C₁₋₇ alkyl of R⁵ can form a ring with any part of Y, wherein the ring optionally contains a carbonyl group;
R⁶ is H or C₁₋₇ alkyl;
R⁸ and R¹¹ are each independently selected from H, C₁₋₇ alkyl, C₂₋₇ alkenyl, and C₃₋₇ cycloalkyl;
X is selected from a bond, —O—C₁₋₇ alkanediyl and C₁₋₇ alkanediyl; and wherein —O—C₁₋₇ alkanediyl or C₁₋₇ alkanediyl of X can form a ring with any part of R⁵ or, when Y is C(O)NR¹⁰R¹² or NR¹⁰R¹², C₁₋₇ alkanediyl of X can form a ring with any part of Y, wherein the ring optionally contains a carbonyl group;
Y is selected from C(O)NR¹⁰R¹², NR¹⁰R¹², C₃₋₇-cycloalkyl optionally containing a heteroatom in the ring selected from O and N wherein if the heteroatom is N it is optionally substituted by R⁸; O-aryl, S-heteroaryl, O-heteroaryl wherein the O-aryl or the O-heteroaryl are optionally substituted by one or more R⁹ and wherein the S-heteroaryl is optionally substituted by one or more R¹⁴; or heteroaryl wherein the heteroaryl is optionally substituted by one or more of R⁸; and wherein, when Y is C(O)NR¹⁰R¹² or NR¹⁰R¹², Y can form a ring with any part of C₁₋₇ alkanediyl of X or any part of C₁₋₇ alkyl of R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R⁹ is selected from H, C₁₋₅ alkyl, halogen, C₁₋₅ alkyl-NR⁸R¹¹, C₁₋₅ alkyl-C(O)NR⁸R¹¹, C₁₋₅ alkyl-C(O)OR⁸, CN, C(O)R⁸, C(O)NR⁸R¹¹, C(O)OR⁸, and OR⁸;
R¹⁰ and R¹² are each independently selected from H, C₁₋₇ alkyl, C₃₋₇ cycloalkyl, C₁₋₃ alkyl-aryl, all these groups optionally substituted by halogen; and
R¹ is C₁₋₇ alkyl.

In some embodiments, Y is selected from NR¹⁰R¹², C₃₋₇-cycloalkyl optionally containing a heteroatom in the ring selected from O and N wherein if the heteroatom is N it is optionally substituted by R⁸; S-heteroaryl, wherein the S-heteroaryl is optionally substituted by one or more R¹⁴; aryl, or heteroaryl wherein the aryl or heteroaryl is optionally substituted by one or more of R⁸; and wherein Y can form a ring with any part of X or R⁵, wherein the ring optionally contains a carbonyl group; with the proviso that when Y is NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, Y is selected from NR¹⁰R¹², C₃₋₇-cycloalkyl optionally containing a heteroatom in the ring selected from O and N wherein if the heteroatom is N it is optionally substituted by R⁸; S-heteroaryl, wherein the S-heteroaryl is optionally substituted by one or more R¹⁴; aryl, or heteroaryl wherein the aryl or heteroaryl is optionally substituted by one or more of R⁸; and wherein, when Y is NR¹⁰R¹², Y can form a ring with any part of X or R⁵, wherein the ring optionally contains a carbonyl group; with the proviso that when Y is NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, the compound is a compound of Formula (I) or Formula (Ia), wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R² is selected from H, C(O)R¹⁴, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by aryl or heteroaryl, wherein the aryl or the heteroaryl is optionally substituted by halogen, C₁₋₄ alkyl or C₃₋₅ cycloalkyl; with the proviso that when R² is C(O)NR¹⁵R¹⁵, both R¹⁵ can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R⁵ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸; and wherein R⁵ can form a ring with any part of X or Y, wherein the ring optionally contains a carbonyl group;
R⁶ is selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl;
X is selected from a bond, C₁₋₇ alkanediyl, C₂₋₇ alkenediyl, C₂₋₇ alkynediyl, C₃₋₆ cycloalkanediyl, C₄₋₆ cycloalkenediyl, —O—, C₁₋₃ alkanediyl-O—, —O—C₁₋₇ alkanediyl, C₁₋₃ alkanediyl-O—C₁₋₇ alkanediyl, C₁₋₇ heteroalkanediyl, or —S—C₁₋₇ alkanediyl; and wherein X can form a ring with any part of R⁵ or Y, wherein the ring optionally contains a carbonyl group;
Y is selected from NR¹⁰R¹², C₃₋₇-cycloalkyl optionally containing a heteroatom in the ring selected from O and N wherein if the heteroatom is N it is optionally substituted by R⁸; S-heteroaryl, wherein the S-heteroaryl is optionally substituted by one or R¹⁴; aryl, or heteroaryl wherein the aryl or heteroaryl is optionally substituted by one or more of R⁸; and wherein Y can form a ring with any part of X or R⁵, wherein the ring optionally contains a carbonyl group; with the proviso that when Y is NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R¹⁰ and R¹² are each independently selected from H, C₁₋₇ alkyl, C₂₋₇ alkenyl, C₂₋₇ alkynyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen, OR⁸, or NR⁸R¹¹;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl; and
R¹⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.

In some embodiments, the compound is a compound of Formula (I) or Formula (Ia), wherein:
R¹ is selected from C₃₋₇ alkyl, C₃₋₇ cycloalkyl, or C₁₋₃ alkyl substituted by aryl or heteroaryl;
R² is selected from H, C(O)R¹⁴, C₁₋₇ alkyl, C₃₋₇ cycloalkyl, C₁₋₅ alkyl-OR⁸; C₁₋₅ alkyl-NHCOR¹³ wherein R¹³ is pentylamino-5-oxopentyl-7-thia-2,4-diazabicyclo[3.3.0]octan-3-one; or C₁₋₃ alkyl substituted by aryl, wherein the aryl is optionally substituted by halogen, C₁₋₄ alkyl or C₃₋₅ cycloalkyl;
R³ and R⁷ are H;
R⁴ is selected from C₃₋₇ alkyl, C₃₋₇ cycloalkyl, or C₁₋₃ alkyl substituted by aryl or heteroaryl;
R⁸ is selected from H, C₁₋₇ alkyl, or OR⁸; and wherein C₁₋₇ alkyl or OR⁸ of R⁵ can form a ring with any part of X or, when Y is NR¹⁰R¹², C₁₋₇ alkyl of R⁵ can form a ring with any part of Y;
R⁶ is H;
R⁸ is selected from H, C₁₋₇ alkyl, C₂₋₇ alkenyl, and C₃₋₇ cycloalkyl;
X is selected from a bond, —O—C₁₋₇ alkanediyl and C₁₋₇ alkanediyl; and wherein —O—C₁₋₇ alkanediyl or C₁₋₇ alkanediyl of X can form a ring with any part of R⁸ or, when Y is NR¹⁰R¹², C₁₋₇ alkanediyl of X can form a ring with any part of Y;
Y is selected from NR¹⁰R¹², C₃₋₇-cycloalkyl optionally containing a heteroatom in the ring selected from O and N wherein if the heteroatom is N it is optionally substituted by R⁸, wherein R⁸ is C₁₋₇ alkyl; S-heteroaryl wherein the S-heteroaryl is optionally substituted by one or more R¹⁴; aryl, or heteroaryl wherein the aryl or the heteroaryl is optionally substituted by one or more of R⁸; and wherein, when Y is NR¹⁰R¹², Y can form a ring with any part of C₁₋₇ alkanediyl of X or any part of C₁₋₇ alkyl of R⁵; with the proviso that when Y is NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R¹⁰ and R¹² are each independently selected from H, C₁₋₇ alkyl, C₃₋₇ cycloalkyl, or C₁₋₃ alkyl-aryl, all these groups optionally substituted by halogen; and
R¹⁴ is C₁₋₇ alkyl.

In some embodiments, the aryl, the heteroaryl or the S-heteroaryl group of any of the compounds of the present disclosure are preferably selected from the group consisting of phenyl, imidazole, pyridine and triazole, more preferably selected from the group consisting of phenyl, imidazole and pyridine.

In some embodiments, Y is selected from NR¹⁰R¹², C₃₋₇-cycloalkyl optionally containing a heteroatom in the ring selected from O and N wherein if the heteroatom is N it is optionally substituted by R⁸; S-heteroaryl, wherein the S-heteroaryl is optionally substituted by one or more R⁴; or heteroaryl wherein the heteroaryl is optionally substituted by one or more of R⁸; and wherein Y can form a ring with any part of X or R⁵, wherein the ring optionally contains a carbonyl group; with the proviso that when Y is NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, Y is selected from NR¹⁰R¹², C₃₋₇-cycloalkyl optionally containing a heteroatom in the ring selected from O and N wherein if the heteroatom is N it is optionally substituted by R⁸; S-heteroaryl, wherein the S-heteroaryl is optionally substituted by one or R⁴; or heteroaryl wherein the heteroaryl is optionally substituted by one or more of R⁸; and wherein, when Y is NR¹⁰R¹², Y can form a ring with any part of X or R⁵, wherein the ring optionally contains a carbonyl group; with the proviso that when Y is NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, the compound is a compound of Formula (I) or Formula (Ia), wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R² is selected from H, C(O)R¹⁴, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by aryl or heteroaryl, wherein the aryl or the heteroaryl is optionally substituted by halogen, C₁₋₄ alkyl or C₃₋₅ cycloalkyl; with the proviso that when R² is C(O)NR¹⁵R¹⁵, both R¹⁵ can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R⁸ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸; and wherein R⁵ can form a ring with any part of X or Y, wherein the ring optionally contains a carbonyl group;
R⁶ is selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl;
X is selected from a bond, C₁₋₇ alkanediyl, C₂₋₇ alkenediyl, C₂₋₇ alkynediyl, C₃₋₆ cycloalkanediyl, C₄₋₆ cycloalkenediyl, —O—, C₁₋₃ alkanediyl-O—, —O—C₁₋₇ alkanediyl, C₁₋₃ alkanediyl-O—C₁₋₇ alkanediyl, C₁₋₇ heteroalkanediyl, or —S—C₁₋₇ alkanediyl; and wherein X can form a ring with any part of R⁵ or Y, wherein the ring optionally contains a carbonyl group;
Y is selected from NR¹⁰R¹², C₃₋₇-cycloalkyl optionally containing a heteroatom in the ring selected from O and N wherein if the heteroatom is N it is optionally substituted by R⁸; S-heteroaryl, wherein the S-heteroaryl is optionally substituted by one or R¹⁴; or heteroaryl wherein the heteroaryl is optionally substituted by one or more of R⁸; and wherein Y can form a ring with any part of X or R⁵, wherein the ring optionally contains a carbonyl group; with the proviso that when Y is NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R¹⁰ and R¹² are each independently selected from H, C₁₋₇ alkyl, C₂₋₇ alkenyl, C₂₋₇ alkynyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen, OR⁸, or NR⁸R¹¹;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl; and
each R⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.

In some embodiments, the compound is a compound of Formula (I) or Formula (Ia), wherein:
R¹ is selected from C₃₋₇ alkyl, C₃₋₇ cycloalkyl, or C₁₋₃ alkyl substituted by aryl or heteroaryl;
R² is selected from H, C(O)R¹⁴, C₁₋₇ alkyl, C₃₋₇ cycloalkyl, C₁₋₅ alkyl-OR⁸; C₁₋₅ alkyl-NHCOR¹³ wherein R¹³ is pentylamino-5-oxopentyl-7-thia-2,4-diazabicyclo[3.3.0]octan-3-one; or C₁₋₃ alkyl substituted by aryl, wherein the aryl is optionally substituted by halogen, C₁₋₄ alkyl or C₃₋₅ cycloalkyl;
R³ and R⁷ are H;
R⁴ is selected from C₃₋₇ alkyl, C₃₋₇ cycloalkyl, or C₁₋₃ alkyl substituted by aryl or heteroaryl;
R⁵ is selected from H, C₁₋₇ alkyl, or OR⁸; and wherein C₁₋₇ alkyl or OR⁸ of R⁵ can form a ring with any part of X or, when Y is NR¹⁰R¹², C₁₋₇ alkyl of R⁵ can form a ring with any part of Y;
R⁶ is H;
R⁸ is selected from H, C₁₋₇ alkyl, C₂₋₇ alkenyl, and C₃₋₇ cycloalkyl;
X is selected from a bond, —O—C₁₋₇ alkanediyl and C₁₋₇ alkanediyl; and wherein —O—C₁₋₇ alkanediyl or C₁₋₇ alkanediyl of X can form a ring with any part of R⁸ or, when Y is NR¹⁰R¹², C₁₋₇ alkanediyl of X can form a ring with any part of Y;
Y is selected from NR¹⁰R¹², C₃₋₇-cycloalkyl optionally containing a heteroatom in the ring selected from O and N wherein if the heteroatom is N it is optionally substituted by R⁸, wherein R⁸ is C₁₋₇ alkyl; S-heteroaryl wherein the S-heteroaryl is optionally substituted by one or more R¹⁴; or heteroaryl wherein the heteroaryl is optionally substituted by one or more of R⁸; and wherein, when Y is NR¹⁰R¹², Y can form a ring with any part of C₁₋₇ alkanediyl of X or any part of C₁₋₇ alkyl of R⁵; with the proviso that when Y is NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸; and
R¹⁴ is C₁₋₇ alkyl.

In some embodiments, the aryl, the heteroaryl or the S-heteroaryl group of the compounds of the present disclosure are preferably selected from the group consisting of phenyl, imidazole, pyridine and triazole, more preferably selected from the group consisting of phenyl, imidazole and pyridine.

In some embodiments, Y is selected from NR¹⁰R¹² and C₃₋₇-cycloalkyl optionally containing a heteroatom in the ring selected from O and N wherein if the heteroatom is N it is optionally substituted by R⁸; and wherein Y can form a ring with any part of X or R⁵; with the proviso that when Y is NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, Y is selected from NR¹⁰R¹² and C₃₋₇-cycloalkyl optionally containing a heteroatom in the ring selected from O and N wherein if the heteroatom is N it is optionally substituted by R⁸; and wherein, when Y is NR¹⁰R¹², Y can form a ring with any part of X or R⁵; with the proviso that when Y is NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, the compound is a compound of Formula (I) or Formula (Ia), wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R² is selected from H, C(O)R¹⁴, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by aryl or heteroaryl, wherein the aryl or the heteroaryl is optionally substituted by halogen, C₁₋₄ alkyl or C₃₋₅ cycloalkyl; with the proviso that when R² is C(O)NR¹⁵R¹⁵, both R¹⁵ can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R⁸ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸; and wherein R⁵ can form a ring with any part of X or Y, wherein the ring optionally contains a carbonyl group;
R⁶ is selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl;
X is selected from a bond, C₁₋₇ alkanediyl, C₂₋₇ alkenediyl, C₂₋₇ alkynediyl, C₃₋₆ cycloalkanediyl, C₄₋₆ cycloalkenediyl, —O—, C₁₋₃ alkanediyl-O—, —O—C₁₋₇ alkanediyl, C₁₋₃ alkanediyl-O—C₁₋₇ alkanediyl, C₁₋₇ heteroalkanediyl, or —S—C₁₋₇ alkanediyl; and wherein X can form a ring with any part of R⁵ or Y, wherein the ring optionally contains a carbonyl group;
Y is selected from NR¹⁰R¹² and C₃₋₇-cycloalkyl optionally containing a heteroatom in the ring selected from O and N wherein if the heteroatom is N it is optionally substituted by R⁸; and wherein Y can form a ring with any part of X or R⁵; with the proviso that when Y is NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R¹⁰ and R¹² are each independently selected from H, C₁₋₇ alkyl, C₂₋₇ alkenyl, C₂₋₇ alkynyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen, OR⁸, or NR⁸R¹¹;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl; and
each R¹⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.

In some embodiments, the compound is a compound of Formula (I) or Formula (Ia), wherein:
R¹ is selected from C₃₋₇ alkyl, C₃₋₇ cycloalkyl, C₁₋₃ alkyl substituted by aryl or heteroaryl;
R² is selected from H, C(O)R¹⁴, wherein R¹⁴ is C₁₋₇ alkyl; C₁₋₇ alkyl, C₃₋₇ cycloalkyl, C₁₋₅ alkyl-OR⁸; C₁₋₅ alkyl-NHCOR¹³ wherein R¹³ is pentylamino-5-oxopentyl-7-thia-2,4-diazabicyclo[3.3.0]octan-3-one; or C₁₋₃ alkyl substituted by aryl, wherein the aryl is optionally substituted by halogen, C₁₋₄ alkyl or C₃₋₅ cycloalkyl;
R³ and R⁷ are H;
R⁴ is selected from C₃₋₇ alkyl, C₃₋₇ cycloalkyl, or C₁₋₃ alkyl substituted by aryl or heteroaryl;
R⁵ is selected from H and C₁₋₇ alkyl; and wherein C₁₋₇ alkyl of R⁵ can form a ring with any part of Y;
R⁶ is H;
R⁸ is selected from H, C₁₋₇ alkyl, C₂₋₇ alkenyl, and C₃₋₇ cycloalkyl;
X is selected from a bond and C₁₋₇ alkanediyl, and wherein C₁₋₇ alkanediyl of X can form a ring with any part of Y;
Y is selected from NR¹⁰R¹² or C₃₋₇-cycloalkyl optionally containing a heteroatom in the ring selected from O and N wherein if the heteroatom is N it is optionally substituted by R⁸, wherein R⁸ is C₁₋₇ alkyl; and wherein, when Y is NR¹⁰R¹², Y can form a ring with any part of C₁₋₇ alkanediyl of X or any part of C₁₋₇ alkyl of R⁵; with the proviso that when Y is NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸; and
R¹⁰ and R¹² are each independently selected from H, C₁₋₇ alkyl, C₃₋₇ cycloalkyl, C₁₋₃ alkyl-aryl, all these groups optionally substituted by halogen.

In some embodiments, the aryl or the heteroaryl group of the compounds of the present disclosure are preferably selected from the group consisting of phenyl, imidazole, pyridine and triazole, more preferably selected from the group consisting of phenyl, imidazole and pyridine.

In some embodiments, the compound is of any one of Formulae (VIIa), (VIIb), (VIIc), (VIId), (VIIe), or (VIIf):

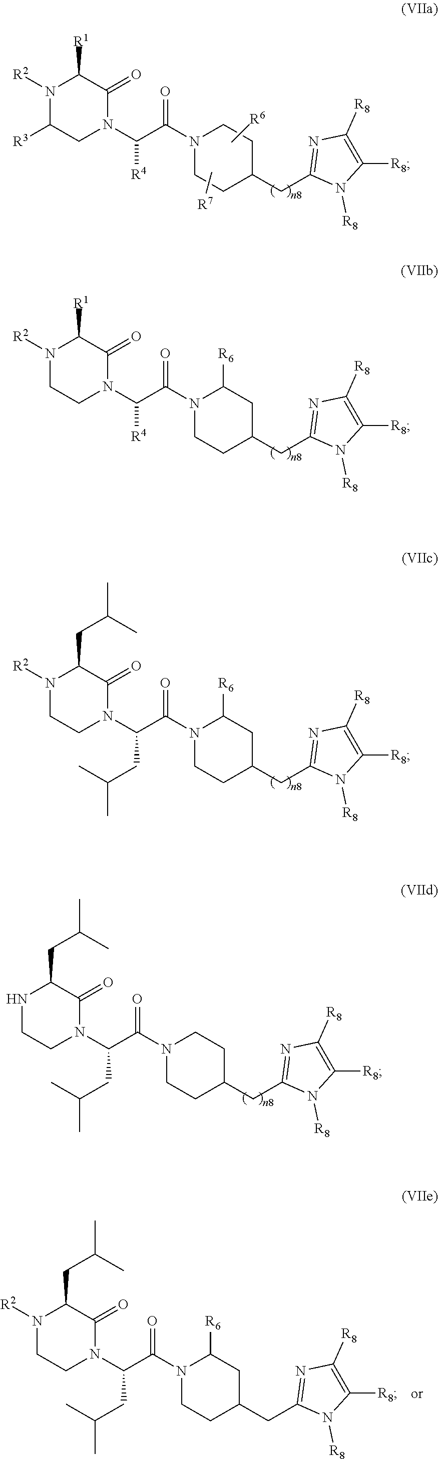

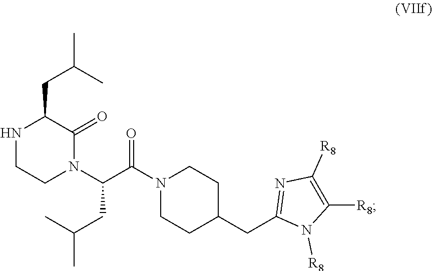

or a pharmaceutically acceptable salt, hydrate, solvate or stereoisomer thereof, wherein n8 is 0, 1, 2, 3, 4, 5, 6, or 7, preferably 1, 2, or 3, and R¹, R², R³, R⁴, R⁶, R⁷, and R⁸ are as described herein.

In some embodiments, the compound is of Formula (VIIa) or a pharmaceutically acceptable salt, hydrate, solvate or stereoisomer thereof, wherein n8 is 0, 1, 2, 3, 4, 5, 6, or 7, preferably 1, 2, or 3, and R¹, R², R³, R⁴, R⁶, R⁷, and R⁸ are as described herein.

In some embodiments, the compound is of Formula (VIIb) or a pharmaceutically acceptable salt, hydrate, solvate or stereoisomer thereof, wherein n8 is 0, 1, 2, 3, 4, 5, 6, or 7, preferably 1, 2, or 3, and R¹, R², R³, R⁴, R⁶, R⁷, and R⁸ are as described herein.

In some embodiments, the compound is of Formula (VIIc) or a pharmaceutically acceptable salt, hydrate, solvate or stereoisomer thereof, wherein n8 is 0, 1, 2, 3, 4, 5, 6, or 7, preferably 1, 2, or 3, and R¹, R², R³, R⁴, R⁶, R⁷, and R⁸ are as described herein.

In some embodiments, the compound is of Formula (VIId) or a pharmaceutically acceptable salt, hydrate, solvate or stereoisomer thereof, wherein n8 is 0, 1, 2, 3, 4, 5, 6, or 7, preferably 1, 2, or 3, and R¹, R², R³, R⁴, R⁶, R⁷, and R⁸ are as described herein.

In some embodiments, the compound is of Formula (VIIe) or a pharmaceutically acceptable salt, hydrate, solvate or stereoisomer thereof, wherein n8 is 0, 1, 2, 3, 4, 5, 6, or 7, preferably 1, 2, or 3, and R¹, R², R³, R⁴, R⁶, R⁷, and R⁸ are as described herein.

In some embodiments, the compound is of Formula (VIIf) or a pharmaceutically acceptable salt, hydrate, solvate or stereoisomer thereof, wherein n8 is 0, 1, 2, 3, 4, 5, 6, or 7, preferably 1, 2, or 3, and R¹, R², R³, R⁴, R⁶, R⁷, and R⁸ are as described herein.

In some embodiments, R⁵ is H and X—Y is

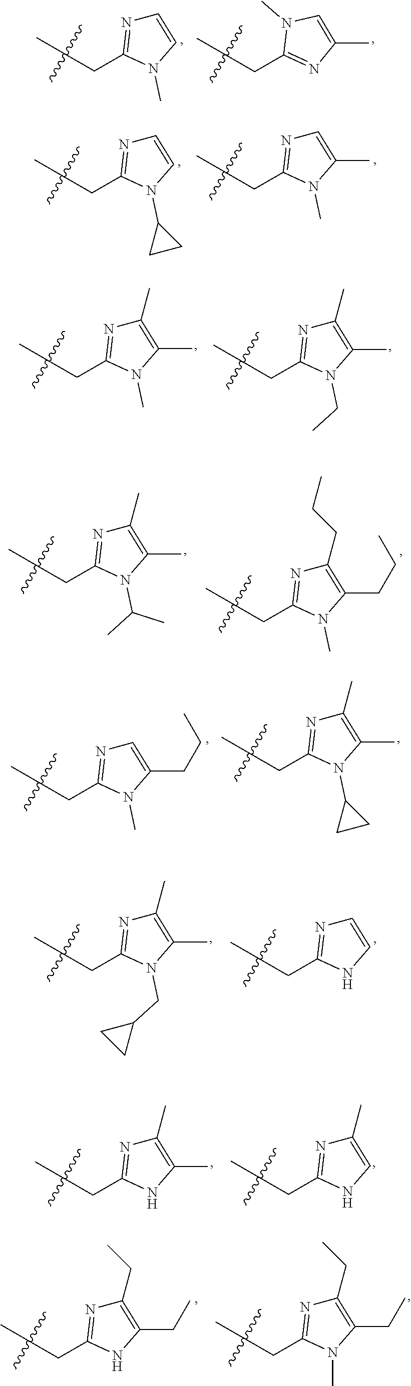

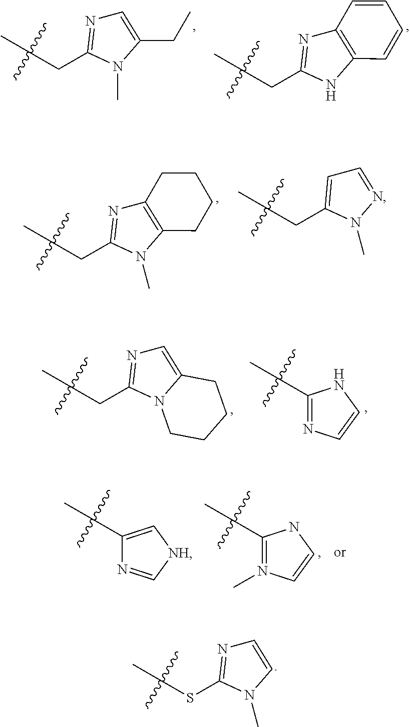

In some embodiments, Y is aryl or heteroaryl, wherein the aryl or the heteroaryl is optionally substituted by one or more of R⁸; or S-heteroaryl, wherein the S-heteroaryl is optionally substituted by one or more R¹⁴.

In some embodiments, the compound is a compound of Formula (I) or Formula (Ia), wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R² is selected from H, C(O)R¹⁴, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by aryl or heteroaryl, wherein the aryl or the heteroaryl is optionally substituted by halogen, C₁₋₄ alkyl or C₃₋₅ cycloalkyl; with the proviso that when R² is C(O)NR¹⁵R¹⁵, both R¹⁵ can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R⁵ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸;
R⁶ is selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl;
X is selected from a bond, C₁₋₇ alkanediyl, C₂₋₇ alkenediyl, C₂₋₇ alkynediyl, C₃₋₆ cycloalkanediyl, C₄₋₆ cycloalkenediyl, —O—, C₁₋₃ alkanediyl-O—, —O—C₁₋₇ alkanediyl, C₁₋₃ alkanediyl-O—C₁₋₇ alkanediyl, C₁₋₇ heteroalkanediyl, or —S—C₁₋₇ alkanediyl;
Y is aryl or heteroaryl, wherein the aryl or the heteroaryl is optionally substituted by one or more of R⁸; or S-heteroaryl, wherein the S-heteroaryl is optionally substituted by one or more R¹⁴;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, C₁₋₃ alkyl substituted by aryl or heteroaryl, wherein the aryl or the heteroaryl is optionally substituted by halogen, C₁₋₄ alkyl or C₃₋₅ cycloalkyl; and
each R⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.

In some embodiments, the compound is a compound of Formula (I) or Formula (Ia), wherein:
R¹ is selected from C₃₋₇ alkyl, C₃₋₇ cycloalkyl, or C₁₋₃ alkyl substituted by aryl or heteroaryl;
R² is selected from H, C(O)R¹⁴, C₁₋₇ alkyl, C₃₋₇ cycloalkyl, C₁₋₅ alkyl-OR⁸; C₁₋₅ alkyl-NHCOR¹³ wherein R¹³ is pentylamino-5-oxopentyl-7-thia-2,4-diazabicyclo[3.3.0]octan-3-one; or C₁₋₃ alkyl substituted by aryl, wherein the aryl is optionally substituted by halogen, C₁₋₄ alkyl or C₃₋₅ cycloalkyl;
R³ and R⁷ are H;
R⁴ is selected from C₃₋₇ alkyl, C₃₋₇ cycloalkyl, or C₁₋₃ alkyl substituted by aryl or heteroaryl;
R⁵ is selected from H and C₁₋₇ alkyl;
R⁶ is H;
R⁸ is selected from H, C₁₋₇ alkyl, C₂₋₇ alkenyl, and C₃₋₇ cycloalkyl;
X is selected from a bond and C₁₋₇ alkanediyl;
Y is aryl or heteroaryl, wherein the aryl or the heteroaryl is optionally substituted by one or more of R⁸; or S-heteroaryl, wherein the S-heteroaryl is optionally substituted by one or more R¹⁴; and
R¹⁴ is C₁₋₇ alkyl.

In some embodiments, the aryl, the heteroaryl or the S-heteroaryl group of any of the compounds of the present disclosure are preferably selected from the group consisting of phenyl, imidazole, pyridine and triazole, more preferably selected from the group consisting of phenyl, imidazole and pyridine.

In some embodiments, Y is heteroaryl, wherein the heteroaryl is optionally substituted by one or more of R⁸; or S-heteroaryl, wherein the S-heteroaryl is optionally substituted by one or more R¹⁴.

In some embodiments, the compound is a compound of Formula (I) or Formula (Ia), wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R² is selected from H, C(O)R¹⁴, C(O)NR¹⁵R¹⁵, C(O)OR⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by aryl or heteroaryl, wherein the aryl or the heteroaryl is optionally substituted by halogen, C₁₋₄ alkyl or C₃₋₅ cycloalkyl; with the proviso that when R² is C(O)NR¹⁵R¹⁵, both R⁵ can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R⁵ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸;
R⁶ is selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl;
X is selected from a bond, C₁₋₇ alkanediyl, C₂₋₇ alkenediyl, C₂₋₇ alkynediyl, C₃₋₆ cycloalkanediyl, C₄₋₆ cycloalkenediyl, —O—, C₁₋₃ alkanediyl-O—, —O—C₁₋₇ alkanediyl, C₁₋₃ alkanediyl-O—C₁₋₇ alkanediyl, C₁₋₇ heteroalkanediyl, or —S—C₁₋₇ alkanediyl;
Y is heteroaryl, wherein the heteroaryl is optionally substituted by one or more of R⁸; or S-heteroaryl, wherein the S-heteroaryl is optionally substituted by one or more R⁴;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl; and
each R¹⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.

In some embodiments, the compound is a compound of Formula (I) or Formula (Ia), wherein:
R¹ is selected from C₃₋₇ alkyl, C₃₋₇ cycloalkyl, or C₁₋₃ alkyl substituted by aryl or heteroaryl;
R² is selected from H, C(O)R¹⁴, C₁₋₇ alkyl, C₃₋₇ cycloalkyl, C₁₋₅ alkyl-OR⁸; C₁₋₅ alkyl-NHCOR¹³ wherein R¹³ is pentylamino-5-oxopentyl-7-thia-2,4-diazabicyclo[3.3.0]octan-3-one; or C₁₋₃ alkyl substituted by aryl, wherein the aryl is optionally substituted by halogen, C₁₋₄ alkyl or C₃₋₅ cycloalkyl;
R³ and R⁷ are H;
R⁴ is selected from C₃₋₇ alkyl, C₃₋₇ cycloalkyl, or C₁₋₃ alkyl substituted by aryl or heteroaryl;
R⁵ is selected from H and C₁₋₇ alkyl;
R⁶ is H;
R⁸ is selected from H, C₁₋₇ alkyl, C₂₋₇ alkenyl, and C₃₋₇ cycloalkyl;
X is selected from a bond and C₁₋₇ alkanediyl;
Y is heteroaryl, wherein the heteroaryl is optionally substituted by one or more of R⁸; or S-heteroaryl, wherein the S-heteroaryl is optionally substituted by one or more R¹⁴; and
R¹⁴ is C₁₋₇alkyl.

In some embodiments, the compound is a compound of Formula (I) or Formula (Ia), wherein:
R¹ is selected from C₃₋₇ alkyl, C₃₋₇ cycloalkyl, or C₁₋₃ alkyl substituted by aryl or heteroaryl;
R² is selected from H, C(O)R¹⁴, C₁₋₇ alkyl, C₃₋₇ cycloalkyl, C₁₋₅ alkyl-OR⁸; C₁₋₅ alkyl-NHCOR¹³ wherein R¹¹ is pentylamino-5-oxopentyl-7-thia-2,4-diazabicyclo[3.3.0]octan-3-one; or C₁₋₃ alkyl substituted by aryl, wherein the aryl is optionally substituted by halogen, C₁₋₄ alkyl or C₃₋₅ cycloalkyl;
R³ and R⁷ are H;
R⁴ is selected from C₃₋₇ alkyl, C₃₋₇ cycloalkyl, or C₁₋₃ alkyl substituted by aryl or heteroaryl;
R⁵ is selected from H, C₁₋₇ alkyl, OR⁸, and O—C₁₋₇ alkyl;
R⁶ is H;
R⁸ is selected from H, C₁₋₇ alkyl, C₂₋₇ alkenyl, and C₃₋₇ cycloalkyl;
X is selected from a bond, C₁₋₇ alkanediyl, —O—, and —O—C₁₋₇ alkanediyl;
Y is heteroaryl, wherein the heteroaryl is optionally substituted by one or more of R⁸; or S-heteroaryl, wherein the S-heteroaryl is optionally substituted by one or more R¹⁴; and
R¹ is C₁₋₇ alkyl.

In some embodiments, the aryl, the heteroaryl or the S-heteroaryl group of any of the compounds of the present disclosure are preferably selected from the group consisting of phenyl, imidazole, pyridine and triazole, more preferably selected from the group consisting of phenyl, imidazole and pyridine.

In some embodiments, R⁵, X and Y form a spirane or spiro compound at the −4 position of the piperidine ring.

In some embodiments, R⁵, X and Y form a spirane or spiro compound at the −4 position of the piperidine ring and R⁵, X and Y form

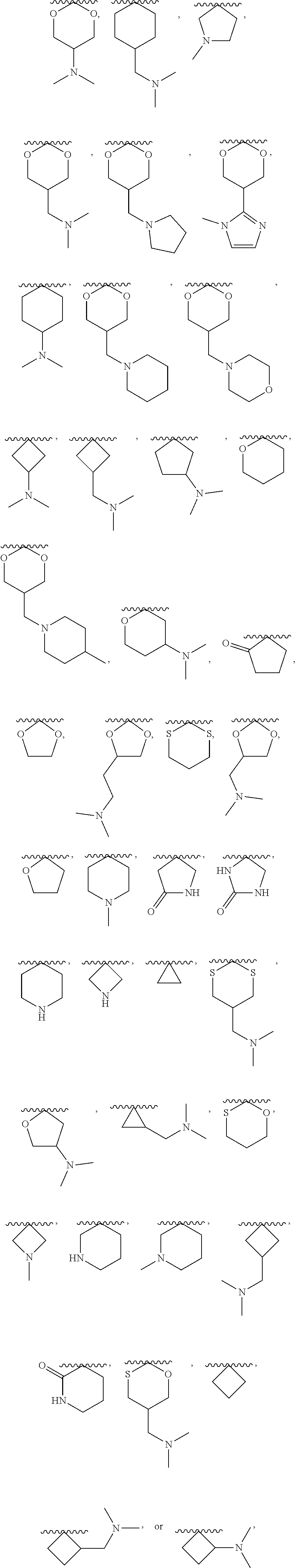

wherein [symbol] indicates the −4 position of the piperidine ring, the common atom of the spirane

In some embodiments, the compound is of any one of Formulae (VIIIa), (VIIIb), (VIIIc), (VIIId), (VIIIe), (VIIIf), (VIIIg), (VIIIh), (VIIIi), (VIIIj), (VIIIk), (VIIIl):

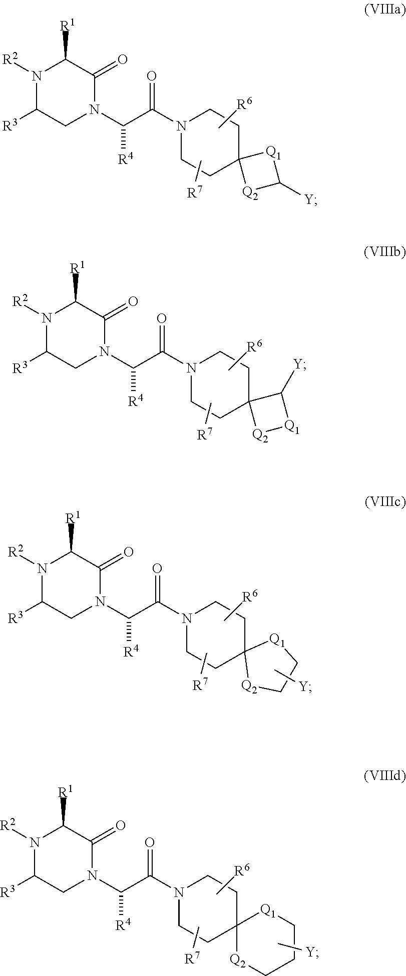

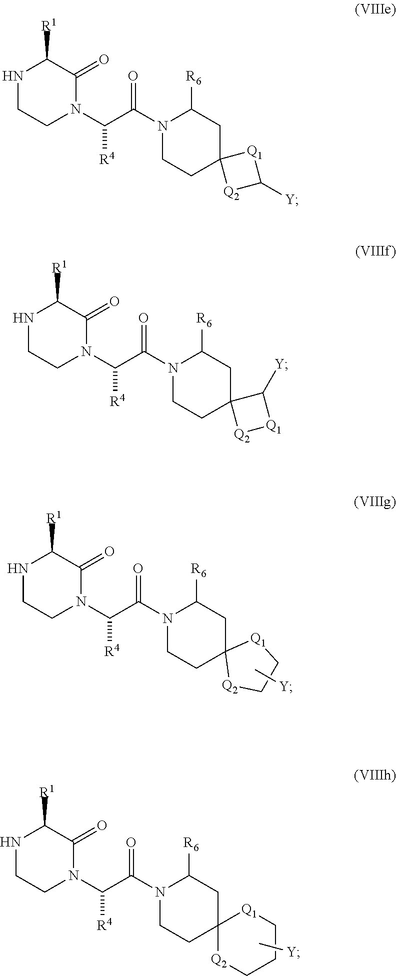

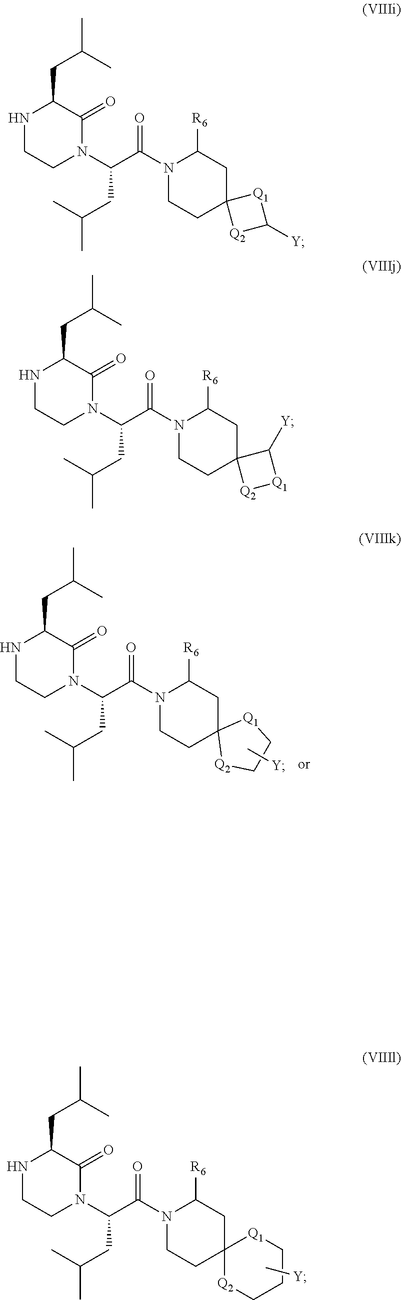

or a pharmaceutically acceptable salt, hydrate, solvate, or stereoisomer thereof, wherein Q₁ and Q₂ are each independently O, S, NR⁸, or CR⁸, and R¹, R², R³, R⁴, R⁶, R⁷, R⁸, and Y are as described herein.

In some embodiments, the compound is of Formula (VIIIa) or a pharmaceutically acceptable salt, hydrate, solvate or stereoisomer thereof, wherein Q₁ and Q₂ are each independently O, S, NR⁸, or CR⁸, and R¹, R², R³, R⁴, R⁶, R⁷, R⁸, and Y are as described herein.

In some embodiments, the compound is of Formula (VIIIb) or a pharmaceutically acceptable salt, hydrate, solvate or stereoisomer thereof, wherein Q₁ and Q₂ are each independently O, S, NR⁸, or CR⁸, and R¹, R², R³, R⁴, R⁶, R⁷, R⁸, and Y are as described herein.

In some embodiments, the compound is of Formula (VIIIc) or a pharmaceutically acceptable salt, hydrate, solvate or stereoisomer thereof, wherein Q₁ and Q₂ are each independently O, S, NR⁸, or CR⁸, and R¹, R², R³, R⁴, R⁶, R⁷, R⁸, and Y are as described herein.

In some embodiments, the compound is of Formula (VIIId) or a pharmaceutically acceptable salt, hydrate, solvate or stereoisomer thereof, wherein Q₁ and Q₂ are each independently O, S, NR⁸, or CR⁸, and R¹, R², R³, R⁴, R⁶, R⁷, R⁸, and Y are as described herein.

In some embodiments, the compound is of Formula (VIIIe) or a pharmaceutically acceptable salt, hydrate, solvate or stereoisomer thereof, wherein Q₁ and Q₂ are each independently O, S, NR⁸, or CR⁸, and R¹, R², R³, R⁴, R⁶, R⁷, R⁸, and Y are as described herein.

In some embodiments, the compound is of Formula (VIIIf) or a pharmaceutically acceptable salt, hydrate, solvate or stereoisomer thereof, wherein Q₁ and Q₂ are each independently O, S, NR⁸, or CR⁸, and R¹, R², R³, R⁴, R⁶, R⁷, R⁸, and Y are as described herein.

In some embodiments, the compound is of Formula (VIIIg) or a pharmaceutically acceptable salt, hydrate, solvate or stereoisomer thereof, wherein Q₁ and Q₂ are each independently O, S, NR⁸, or CR⁸, and R¹, R², R³, R⁴, R⁶, R⁷, R⁸, and Y are as described herein.

In some embodiments, the compound is of Formula (VIIIh) or a pharmaceutically acceptable salt, hydrate, solvate or stereoisomer thereof, wherein Q₁ and Q₂ are each independently O, S, NR⁸, or CR⁸, and R¹, R², R³, R⁴, R⁶, R⁷, R⁸, and Y are as described herein.

In some embodiments, the compound is of Formula (VIIIi) or a pharmaceutically acceptable salt, hydrate, solvate or stereoisomer thereof, wherein Q₁ and Q₂ are each independently O, S, NR⁸, or CR⁸, and R¹, R², R³, R⁴, R⁶, R⁷, R⁸, and Y are as described herein.

In some embodiments, the compound is of Formula (VIIIj) or a pharmaceutically acceptable salt, hydrate, solvate or stereoisomer thereof, wherein Q₁ and Q₂ are each independently O, S, NR⁸, or CR⁸, and R¹, R², R³, R⁴, R⁶, R⁷, R⁸, and Y are as described herein.

In some embodiments, the compound is of Formula (VIIIk) or a pharmaceutically acceptable salt, hydrate, solvate or stereoisomer thereof, wherein Q₁ and Q₂ are each independently O, S, NR⁸, or CR⁸, and R¹, R², R³, R⁴, R⁶, R⁷, R⁸, and Y are as described herein.

In some embodiments, the compound is of Formula (VIIIl) or a pharmaceutically acceptable salt, hydrate, solvate or stereoisomer thereof, wherein Q₁ and Q₂ are each independently O, S, NR⁸, or CR⁸, and R¹, R², R³, R⁴, R⁶, R⁷, R⁸, and Y are as described herein.

In some embodiments, the compound is of any one of Formulae (VIIIa1), (VIIIb1), (VIIIc1), (VIIId1), (VIIIe1), (VIIIf1), (VIIIg1), (VIIIh1), (VIIIi1), (VIIIj1), (VIIIk1), (VIIIl1).

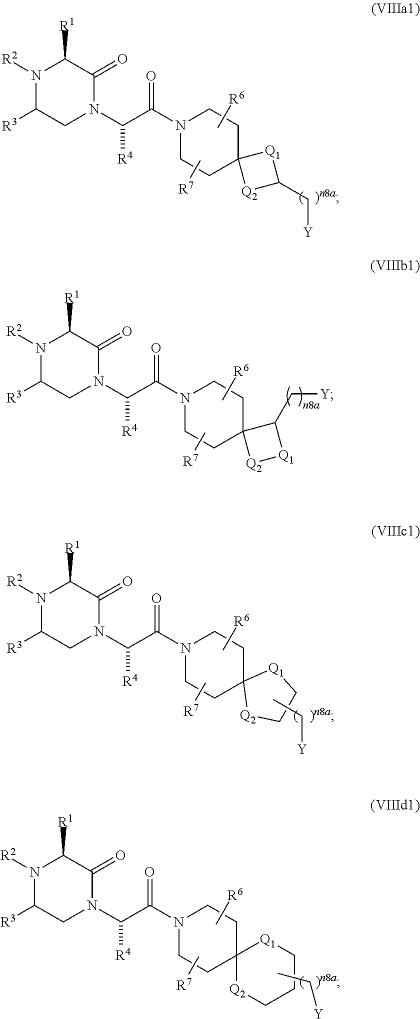

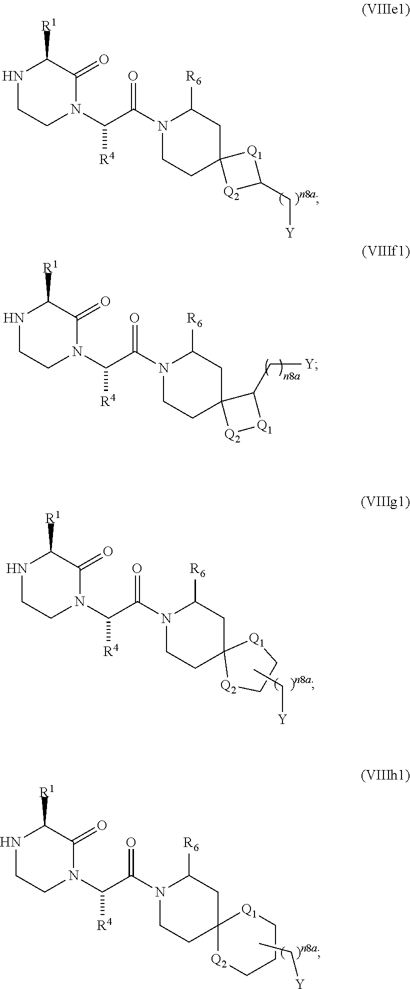

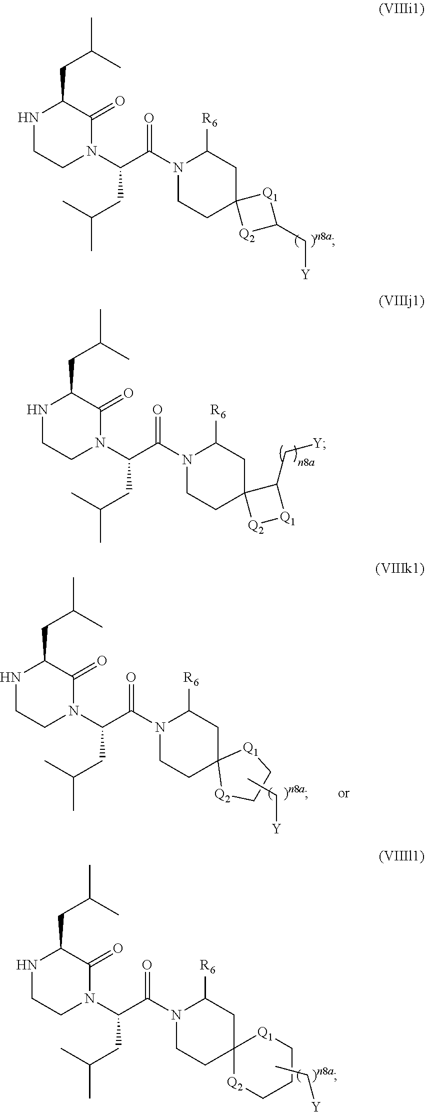

or a pharmaceutically acceptable salt, hydrate, solvate, or stereoisomer thereof, wherein Q₁ and Q₂ are each independently O, S, NR⁸, or CR⁸, n8a is 0, 1, 2, 3, 4, 5, 6, or 7, preferably 1, 2, or 3, and R¹, R², R³, R⁴, R⁶, R⁷, R⁸, and Y are as described herein.

In some embodiments, the compound is of Formula (VIIIa1) or a pharmaceutically acceptable salt, hydrate, solvate or stereoisomer thereof, wherein Q₁ and Q₂ are each independently O, S, NR⁸, or CR⁸, n8a is 0, 1, 2, 3, 4, 5, 6, or 7, preferably 1, 2, or 3, and R¹, R², R³, R⁴, R⁶, R⁷, R⁸, and Y are as described herein.

In some embodiments, the compound is of Formula (VIIIb1) or a pharmaceutically acceptable salt, hydrate, solvate or stereoisomer thereof, wherein Q₁ and Q₂ are each independently O, S, NR⁸, or CR⁸, n8a is 0, 1, 2, 3, 4, 5, 6, or 7, preferably 1, 2, or 3, and R¹, R², R³, R⁴, R⁶, R⁷, R⁸, and Y are as described herein.

In some embodiments, the compound is of Formula (VIIIc1) or a pharmaceutically acceptable salt, hydrate, solvate or stereoisomer thereof, wherein Q₁ and Q₂ are each independently O, S, NR⁸, or CR⁸, n8a is 0, 1, 2, 3, 4, 5, 6, or 7, preferably 1, 2, or 3, and R¹, R², R³, R⁴, R⁶, R⁷, R⁸, and Y are as described herein.

In some embodiments, the compound is of Formula (VIIId1) or a pharmaceutically acceptable salt, hydrate, solvate or stereoisomer thereof, wherein Q₁ and Q₂ are each independently O, S, NR⁸, or CR⁸, n8a is 0, 1, 2, 3, 4, 5, 6, or 7, preferably 1, 2, or 3, and R¹, R², R³, R⁴, R⁶, R⁷, R⁸, and Y are as described herein.

In some embodiments, the compound is of Formula (VIIIe1) or a pharmaceutically acceptable salt, hydrate, solvate or stereoisomer thereof, wherein Q₁ and Q₂ are each independently O, S, NR⁸, or CR⁸, n8a is 0, 1, 2, 3, 4, 5, 6, or 7, preferably 1, 2, or 3, and R¹, R², R³, R⁴, R⁶, R⁷, R⁸, and Y are as described herein.

In some embodiments, the compound is of Formula (VIIIf1) or a pharmaceutically acceptable salt, hydrate, solvate or stereoisomer thereof, wherein Q₁ and Q₂ are each independently O, S, NR⁸, or CR⁸, n8a is 0, 1, 2, 3, 4, 5, 6, or 7, preferably 1, 2, or 3, and R¹, R², R³, R⁴, R⁶, R⁷, R⁸, and Y are as described herein.

In some embodiments, the compound is of Formula (VIIIg1) or a pharmaceutically acceptable salt, hydrate, solvate or stereoisomer thereof, wherein Q₁ and Q₂ are each independently O, S, NR⁸, or CR⁸, n8a is 0, 1, 2, 3, 4, 5, 6, or 7, preferably 1, 2, or 3, and R¹, R², R³, R⁴, R⁶, R⁷, R⁸, and Y are as described herein.

In some embodiments, the compound is of Formula (VIIIh1) or a pharmaceutically acceptable salt, hydrate, solvate or stereoisomer thereof, wherein Q₁ and Q₂ are each independently O, S, NR⁸, or CR⁸, n8a is 0, 1, 2, 3, 4, 5, 6, or 7, preferably 1, 2, or 3, and R¹, R², R³, R⁴, R⁶, R⁷, R⁸, and Y are as described herein.

In some embodiments, the compound is of Formula (VIIIi1) or a pharmaceutically acceptable salt, hydrate, solvate or stereoisomer thereof, wherein Q₁ and Q₂ are each independently O, S, NR⁸, or CR⁸, n8a is 0, 1, 2, 3, 4, 5, 6, or 7, preferably 1, 2, or 3, and R¹, R², R³, R⁴, R⁶, R⁷, R⁸, and Y are as described herein.

In some embodiments, the compound is of Formula (VIIIj1) or a pharmaceutically acceptable salt, hydrate, solvate or stereoisomer thereof, wherein Q₁ and Q₂ are each independently O, S, NR⁸, or CR⁸, n8a is 0, 1, 2, 3, 4, 5, 6, or 7, preferably 1, 2, or 3, and R¹, R², R³, R⁴, R⁶, R⁷, R⁸, and Y are as described herein.

In some embodiments, the compound is of Formula (VIIIk1) or a pharmaceutically acceptable salt, hydrate, solvate or stereoisomer thereof, wherein Q₁ and Q₂ are each independently O, S, NR⁸, or CR⁸, n8a is 0, 1, 2, 3, 4, 5, 6, or 7, preferably 1, 2, or 3, and R¹, R², R³, R⁴, R⁶, R⁷, R⁸, and Y are as described herein.

In some embodiments, the compound is of Formula (VIIIl1) or a pharmaceutically acceptable salt, hydrate, solvate or stereoisomer thereof, wherein Q₁ and Q₂ are each independently O, S, NR⁸, or CR⁸, n8a is 0, 1, 2, 3, 4, 5, 6, or 7, preferably 1, 2, or 3, and R¹, R², R³, R⁴, R⁶, R⁷, R⁸, and Y are as described herein.

In some embodiments, Y is NR¹⁰R¹², wherein R¹⁰ and R¹² can form a ring wherein the ring contains the N of NR¹⁰R¹² and optionally one further heteroatom selected from O and N, wherein if the one further heteroatom is N, it is optionally substituted by R⁸.

In some embodiments, the compound is of any one of Formulae (IXa), (IXb), (IXc), or (IXd).

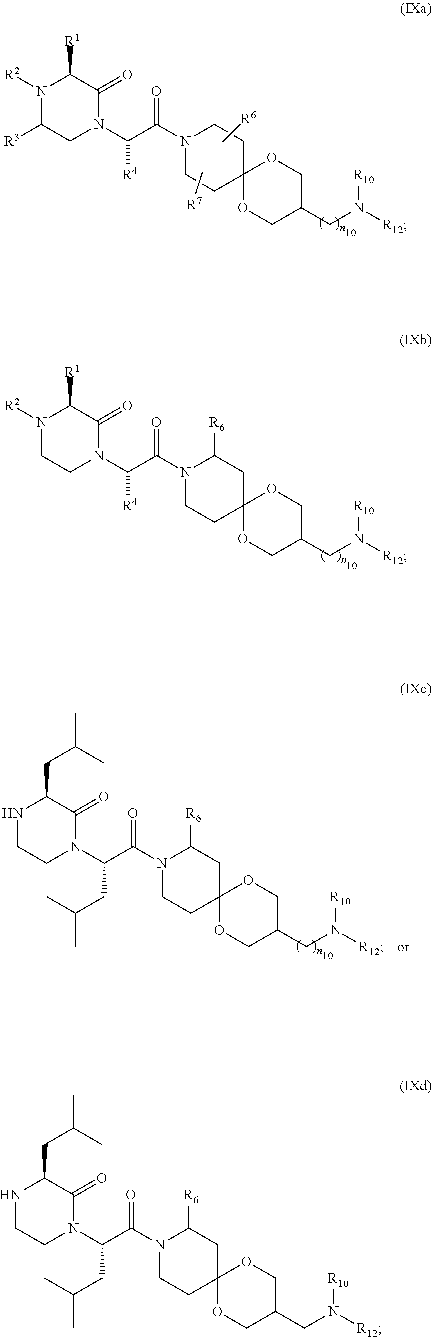

or a pharmaceutically acceptable salt, hydrate, solvate, or stereoisomer thereof, wherein n10 is 0, 1, 2, 3, 4, 5, 6, or 7, preferably 1, 2, or 3, and R¹, R², R³, R⁴, R⁶, R⁷, R¹⁰, R¹² and Y are as described herein.

In some embodiments, the compound is of Formula (IXa) or a pharmaceutically acceptable salt, hydrate, solvate, or stereoisomer thereof, wherein n10 is 0, 1, 2, 3, 4, 5, 6, or 7, preferably 1, 2, or 3, and R¹, R², R³, R⁴, R⁶, R⁷, R¹⁰, R¹² and Y are as described herein.

In some embodiments, the compound is of Formula (IXb) or a pharmaceutically acceptable salt, hydrate, solvate, or stereoisomer thereof, wherein n10 is 0, 1, 2, 3, 4, 5, 6, or 7, preferably 1, 2, or 3, and R¹, R², R³, R⁴, R⁶, R⁷, R¹⁰, R¹² and Y are as described herein.

In some embodiments, the compound is of Formula (IXc) or a pharmaceutically acceptable salt, hydrate, solvate, or stereoisomer thereof, wherein n10 is 0, 1, 2, 3, 4, 5, 6, or 7, preferably 1, 2, or 3, and R¹, R², R³, R⁴, R⁶, R⁷, R¹⁰, R¹² and Y are as described herein.

In some embodiments, the compound is of Formula (IXd) or a pharmaceutically acceptable salt, hydrate, solvate, or stereoisomer thereof, wherein n10 is 0, 1, 2, 3, 4, 5, 6, or 7, preferably 1, 2, or 3, and R¹, R², R³, R⁴, R⁶, R⁷, R¹⁰, R¹² and Y are as described herein.

In some embodiments, the compound is of any one of Formulae (IXa1), (IXb1), (IXc1), or (IXd1):

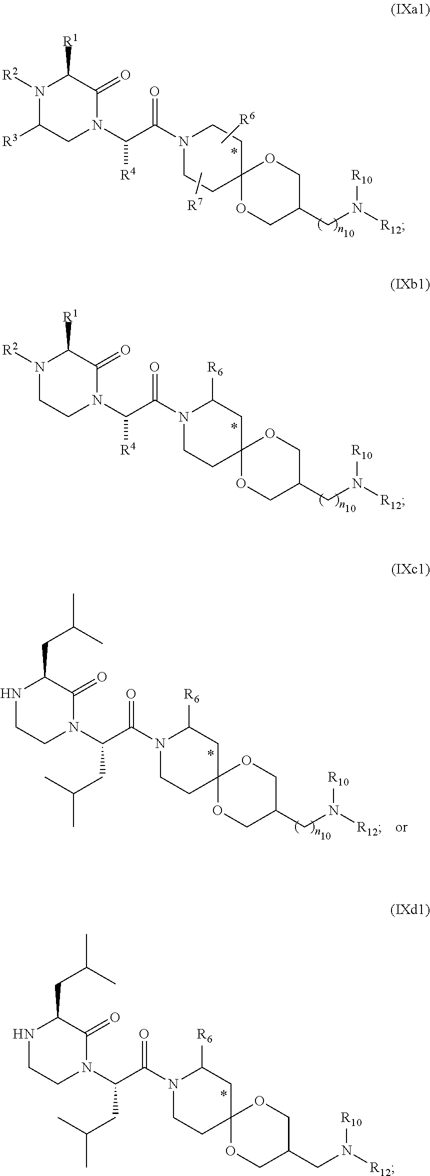

or a pharmaceutically acceptable salt, hydrate, solvate, or stereoisomer thereof, wherein n10 is 0, 1, 2, 3, 4, 5, 6, or 7, preferably 1, 2, or 3, R¹, R², R³, R⁴, R⁶, R⁷, R¹⁰, R¹² and Y are as described herein, and * indicates the Z-isomer of the spiro compound.

In some embodiments, the compound is of Formula (IXa1) or a pharmaceutically acceptable salt, hydrate, solvate, or stereoisomer thereof, wherein n10 is 0, 1, 2, 3, 4, 5, 6, or 7, preferably 1, 2, or 3, R¹, R², R³, R⁴, R⁶, R⁷, R¹⁰, R¹² and Y are as described herein, and * indicates the Z-isomer of the spiro compound.

In some embodiments, the compound is of Formula (IXb1) or a pharmaceutically acceptable salt, hydrate, solvate, or stereoisomer thereof, wherein n10 is 0, 1, 2, 3, 4, 5, 6, or 7, preferably 1, 2, or 3, R¹, R², R³, R⁴, R⁶, R⁷, R¹⁰, R¹² and Y are as described herein, and * indicates the Z-isomer of the spiro compound.

In some embodiments, the compound is of Formula (IXc1) or a pharmaceutically acceptable salt, hydrate, solvate, or stereoisomer thereof, wherein n10 is 0, 1, 2, 3, 4, 5, 6, or 7, preferably 1, 2, or 3, R¹, R², R³, R⁴, R⁶, R⁷, R¹⁰, R¹² and Y are as described herein, and * indicates the Z-isomer of the spiro compound.

In some embodiments, the compound is of Formula (IXd1) or a pharmaceutically acceptable salt, hydrate, solvate, or stereoisomer thereof, wherein n10 is 0, 1, 2, 3, 4, 5, 6, or 7, preferably 1, 2, or 3, R¹, R², R³, R⁴, R⁶, R⁷, R¹⁰, R¹² and Y are as described herein, and * indicates the Z-isomer of the spiro compound.

In some embodiments, the compound is a compound of Formula (I) or Formula (Ia), wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R² is selected from H, C(O)R¹⁴, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by aryl or heteroaryl, wherein the aryl or the heteroaryl is optionally substituted by halogen, C₁₋₄ alkyl or C₃₋₅ cycloalkyl; with the proviso that when R² is C(O)NR¹⁵R¹⁵, both R¹⁵ can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R⁵ is selected from C₁₋₇ alkyl, OR⁸, or SR⁸; wherein C₁₋₇ alkyl, OR⁸ or SR of R⁵ can form a ring with any part of X;
R⁶ is selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl;
X is selected from —O—C₁₋₇ alkanediyl, —S—C₁₋₇ alkanediyl, or C₁₋₇ alkanediyl, and wherein —O—C₁₋₇ alkanediyl, —S—C₁₋₇ alkanediyl or C₁₋₇ alkanediyl of X can form a ring with any part of R⁵;
Y is NR¹⁰R¹², wherein R¹⁰ and R¹² can form a ring wherein the ring contains the N of NR¹⁰R¹² and optionally one further heteroatom selected from O and N, wherein if the one further heteroatom is N, it is optionally substituted by R⁸;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl; and
each R¹⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.

In some embodiments, the compound is a compound of Formula (I) or Formula (Ia), wherein:
R¹ is selected from C₃₋₇ alkyl, C₃₋₇ cycloalkyl, or C₁₋₃ alkyl substituted by aryl or heteroaryl;
R² is selected from H, C(O)R¹⁴, C₁₋₇ alkyl, C₃₋₇ cycloalkyl, C₁₋₅ alkyl-OR⁸; C₁₋₅ alkyl-NHCOR¹³ wherein R¹³ is pentylamino-5-oxopentyl-7-thia-2,4-diazabicyclo[3.3.0]octan-3-one; or C₁₋₃ alkyl substituted by aryl, wherein the aryl is optionally substituted by halogen, C₁₋₄ alkyl or C₃₋₅ cycloalkyl;
R³ and R⁷ are H;
R⁴ is selected from C₃₋₇ alkyl, C₃₋₇ cycloalkyl, C₁₋₃ alkyl substituted by aryl or heteroaryl;
R⁵ is OR⁸, and OR⁸ of R⁵ can form a ring with any part of X;
R⁶ is H;
R⁸ and R¹¹ are C₁₋₇ alkyl;
X is —O—C₁₋₇ alkanediyl and wherein —O—C₁₋₇ alkanediyl of X can form a ring with any part of R⁵; and
Y is NR¹⁰R¹², wherein R¹⁰ and R¹² can form a ring wherein the ring contains the N of NR¹⁰R¹² and optionally one further heteroatom selected from O and N, wherein if the one further heteroatom is N, it is optionally substituted by R⁸.

Some preferred embodiments of the present application relate to the compounds having one of the following structures or being one of the following compounds, pharmaceutically-acceptable salts, hydrates, solvates, or stereoisomers thereof:

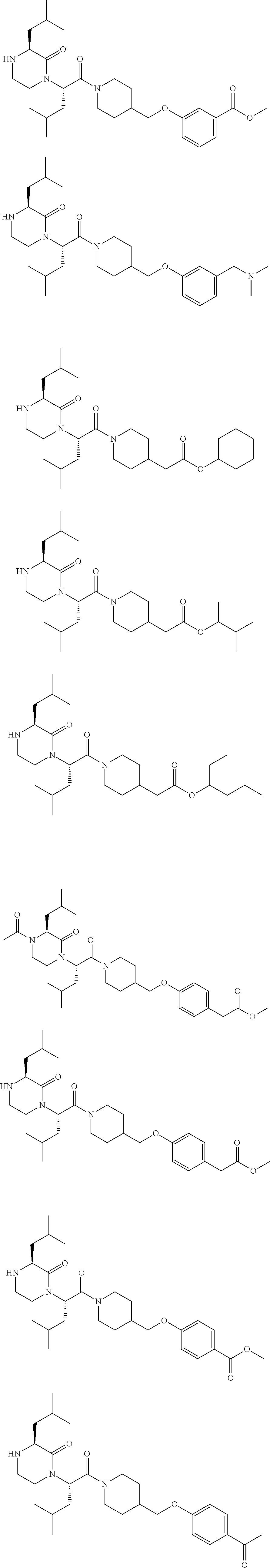

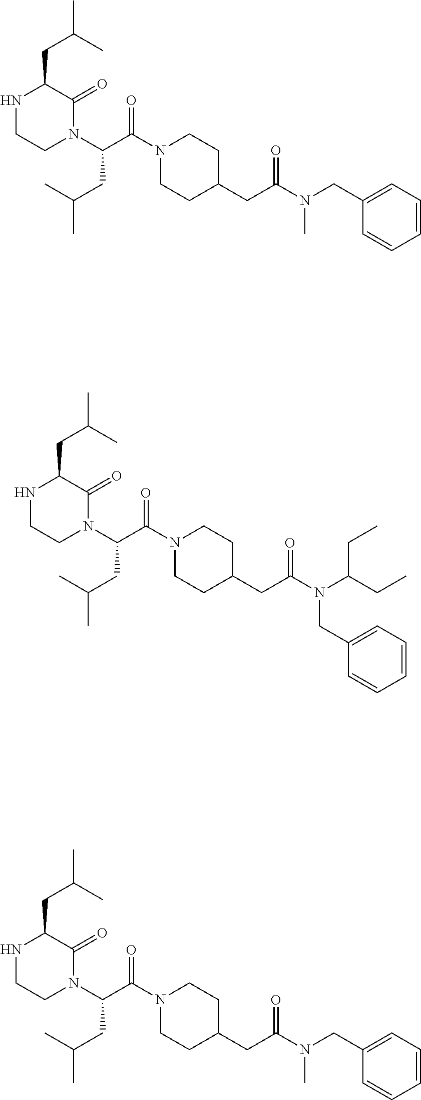 [SMILES: CC(C)C[C@@H]1NCCN([C@@H](CC(C)C)C(=O)N2CCC(CC(=O)N(C)Cc3ccccc3)CC2)C1=O ; CCC(CC)N(Cc1ccccc1)C(=O)CC1CCN(C(=O)[C@H](CC(C)C)N2CCN[C@@H](CC(C)C)C2=O)CC1 ; CC(C)C[C@@H]1NCCN([C@@H](CC(C)C)C(=O)N2CCC(CC(=O)N(C)Cc3ccccc3)CC2)C1=O]

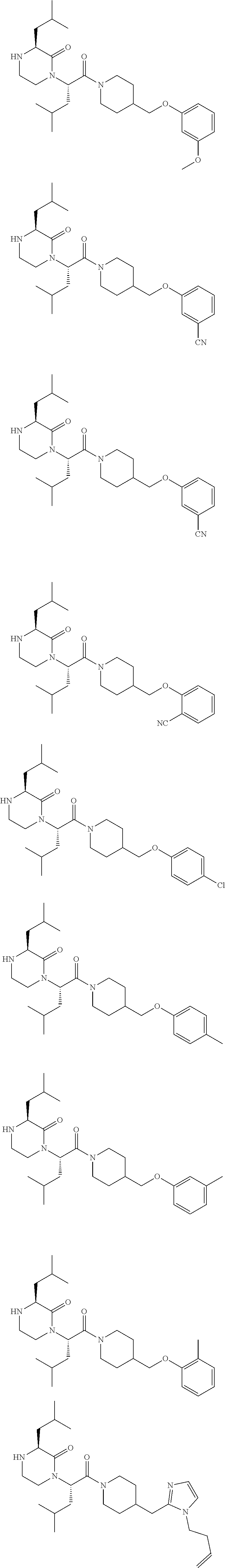

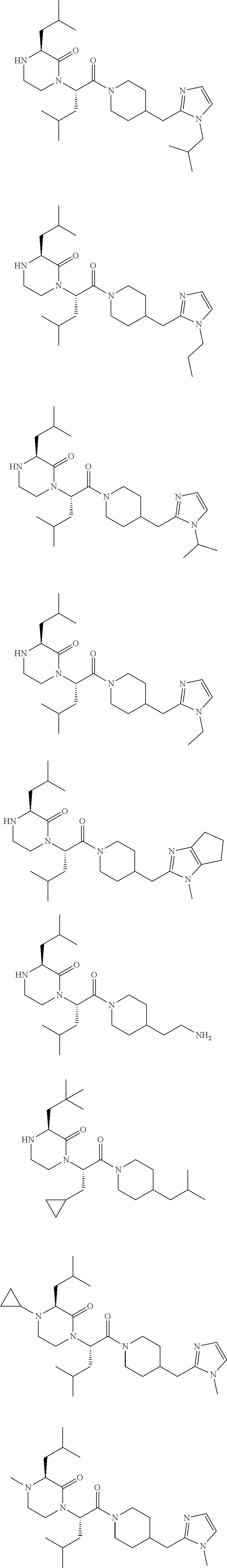

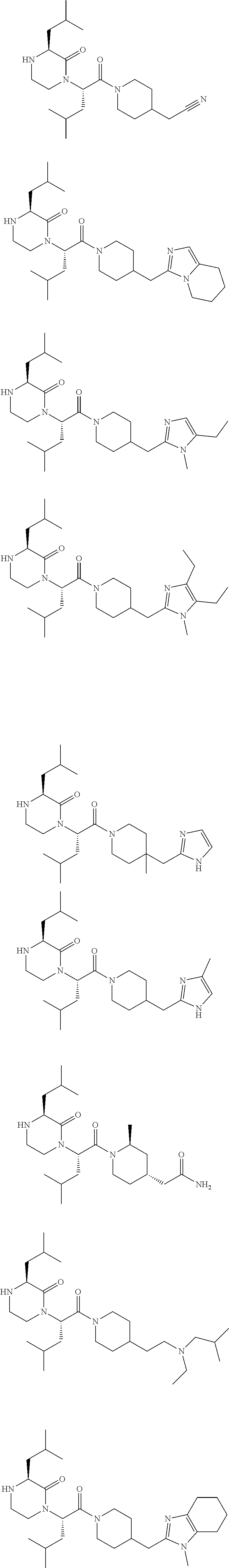

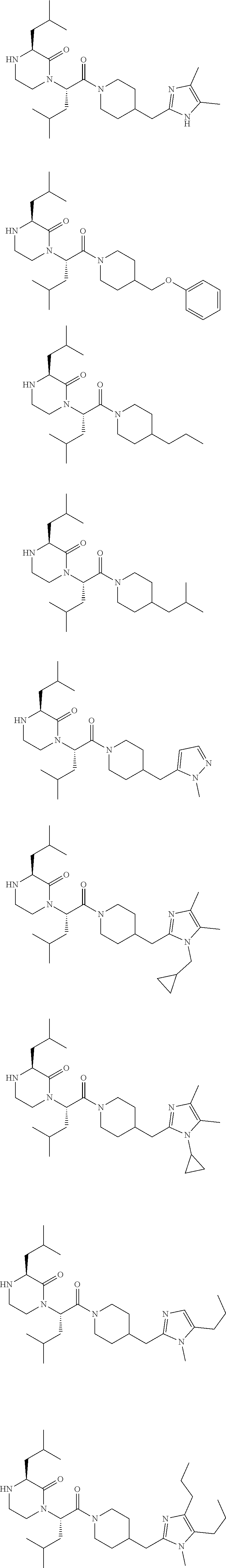

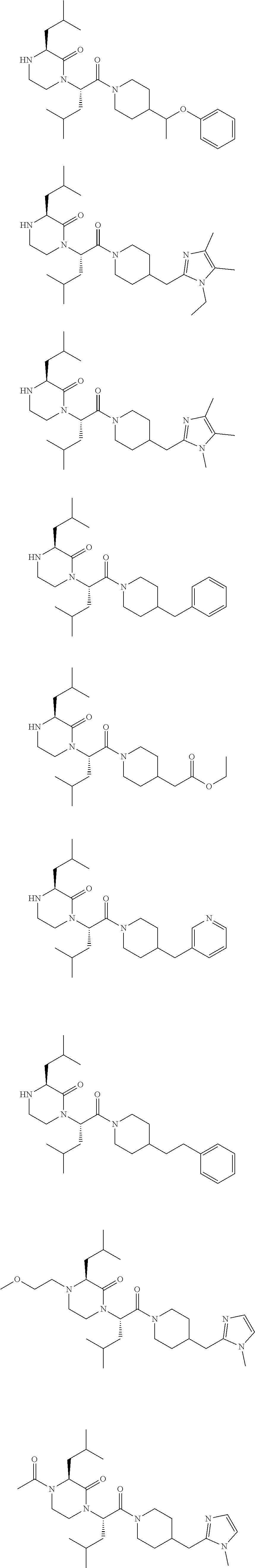

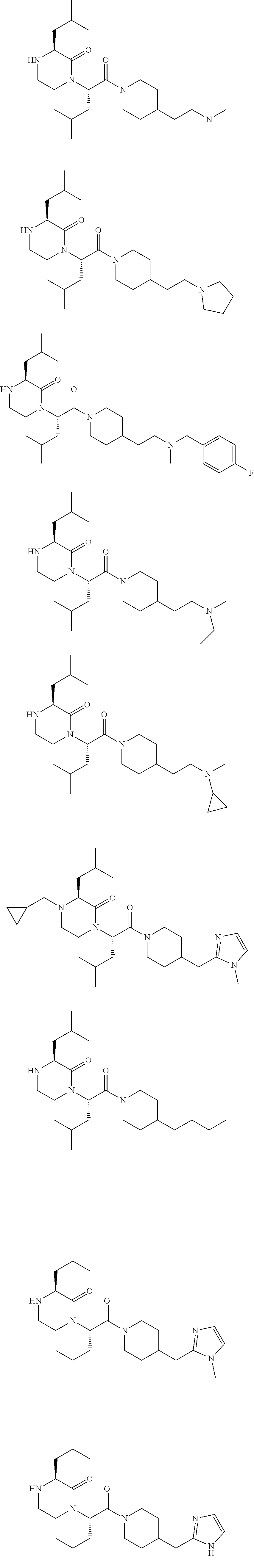

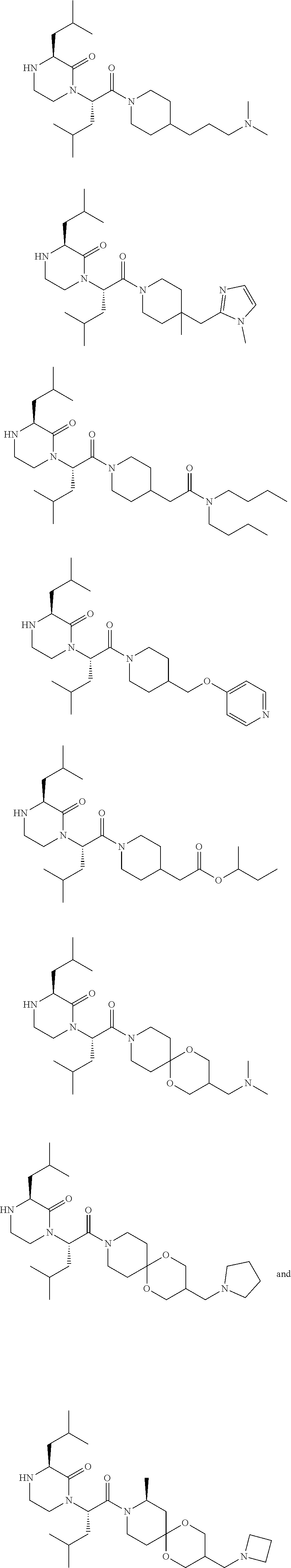

Some preferred embodiments of the present application relate to the compounds having one of the following structures or being one of the following compounds, pharmaceutically-acceptable salts, hydrates, solvates, or stereoisomers thereof:

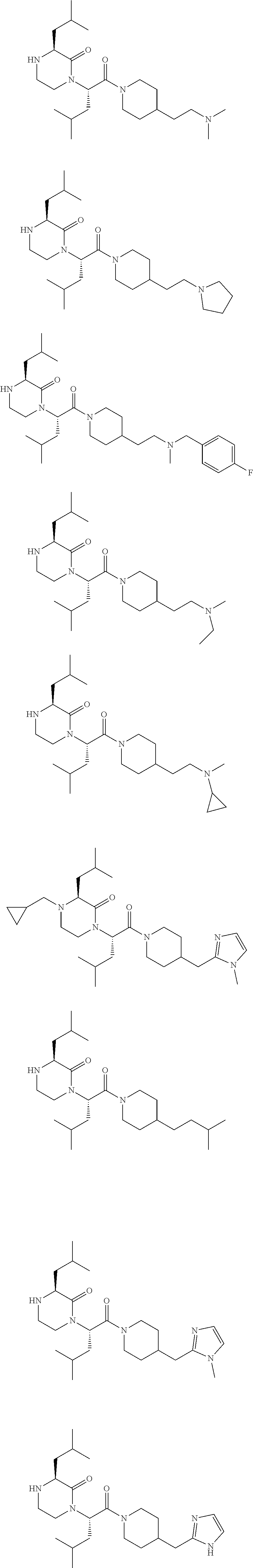

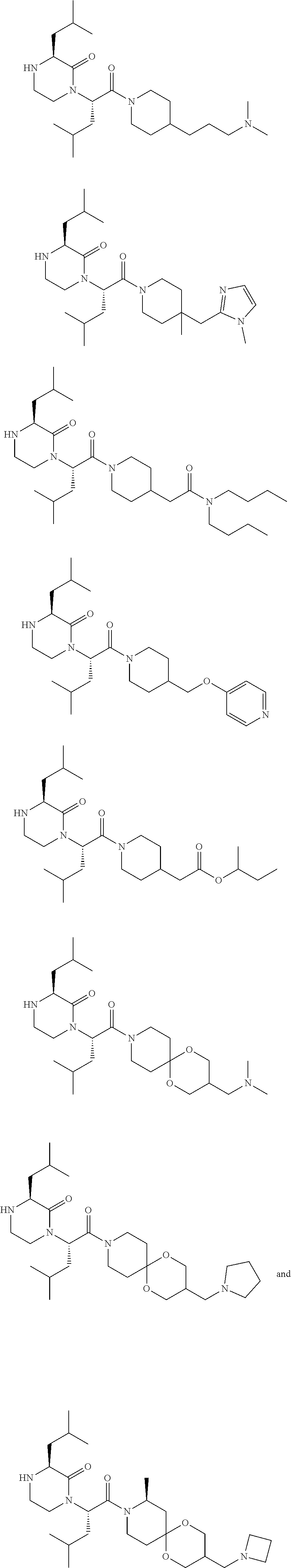

Some embodiments of the present application relate to the compounds having one of the following structures or being one of the following compounds, pharmaceutically-acceptable salts, hydrates, solvates, or stereoisomers thereof:

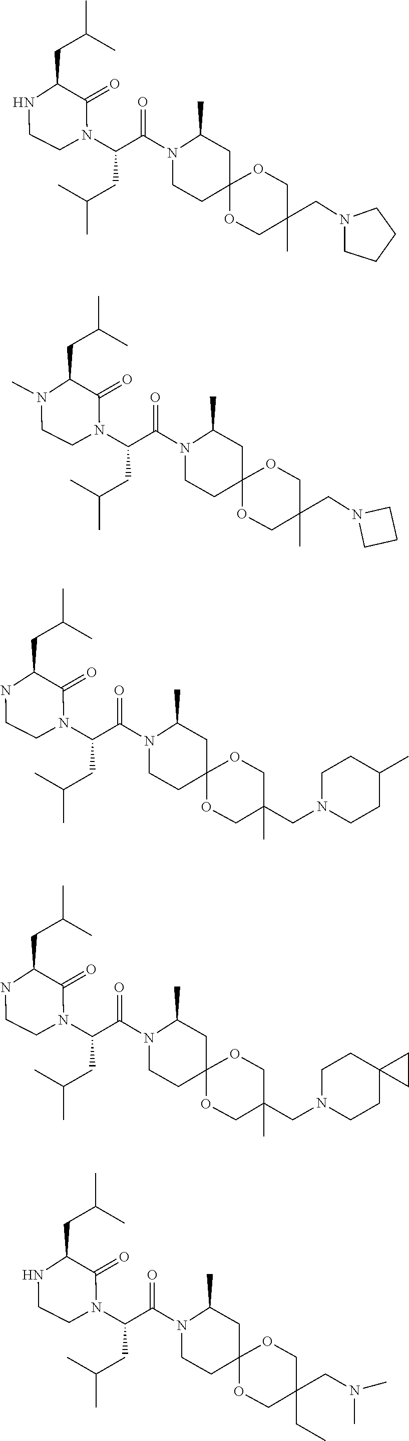 [SMILES: CC(C)C[C@@H]1NCCN([C@@H](CC(C)C)C(=O)N2CCC3(C[C@@H]2C)OCC(C)(CN2CCCC2)CO3)C1=O ; CC(C)C[C@H]1C(=O)N([C@@H](CC(C)C)C(=O)N2CCC3(C[C@@H]2C)OCC(C)(CN2CCC2)CO3)CCN1C ; CC(C)C[C@@H]1NCCN([C@@H](CC(C)C)C(=O)N2CCC3(C[C@@H]2C)OCC(C)(CN2CCC(C)CC2)CO3)C1=O ; CC(C)C[C@@H]1NCCN([C@@H](CC(C)C)C(=O)N2CCC3(C[C@@H]2C)OCC(C)(CN2CCC4(CC2)CC4)CO3)C1=O ; CCC1(CN(C)C)COC2(CCN(C(=O)[C@H](CC(C)C)N3CCN[C@@H](CC(C)C)C3=O)[C@@H](C)C2)OC1]

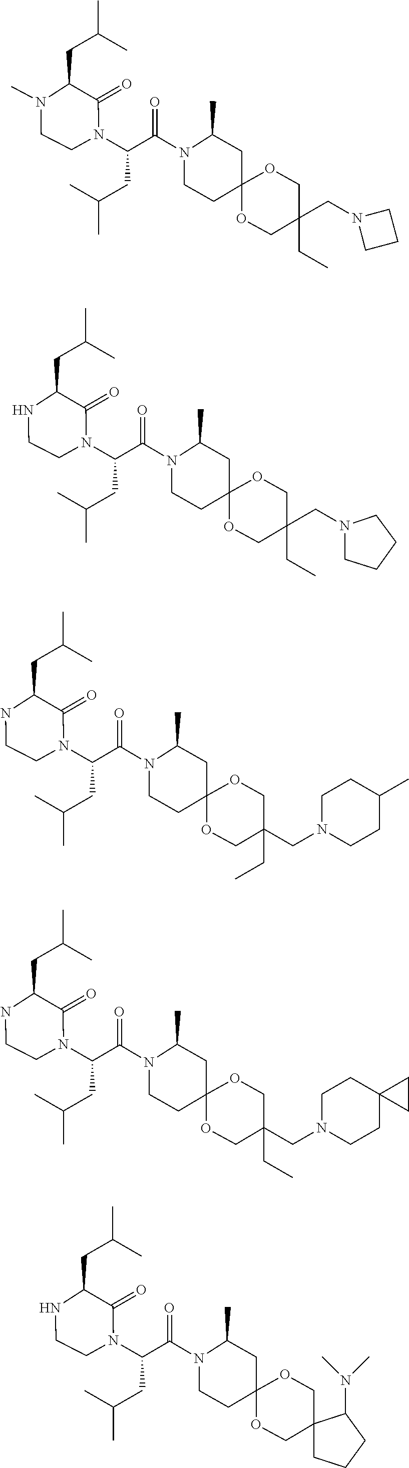 [SMILES: CCC1(CN2CCC2)COC2(CCN(C(=O)[C@H](CC(C)C)N3CCN(C)[C@@H](CC(C)C)C3=O)[C@@H](C)C2)OC1 ; CCC1(CN2CCCC2)COC2(CCN(C(=O)[C@H](CC(C)C)N3CCN[C@@H](CC(C)C)C3=O)[C@@H](C)C2)OC1 ; CCC1(CN2CCC(C)CC2)COC2(CCN(C(=O)[C@H](CC(C)C)N3CCN[C@@H](CC(C)C)C3=O)[C@@H](C)C2)OC1 ; CCC1(CN2CCC3(CC2)CC3)COC2(CCN(C(=O)[C@H](CC(C)C)N3CCN[C@@H](CC(C)C)C3=O)[C@@H](C)C2)OC1 ; CC(C)C[C@@H]1NCCN([C@@H](CC(C)C)C(=O)N2CCC3(C[C@@H]2C)OCC2(CCCC2N(C)C)CO3)C1=O]

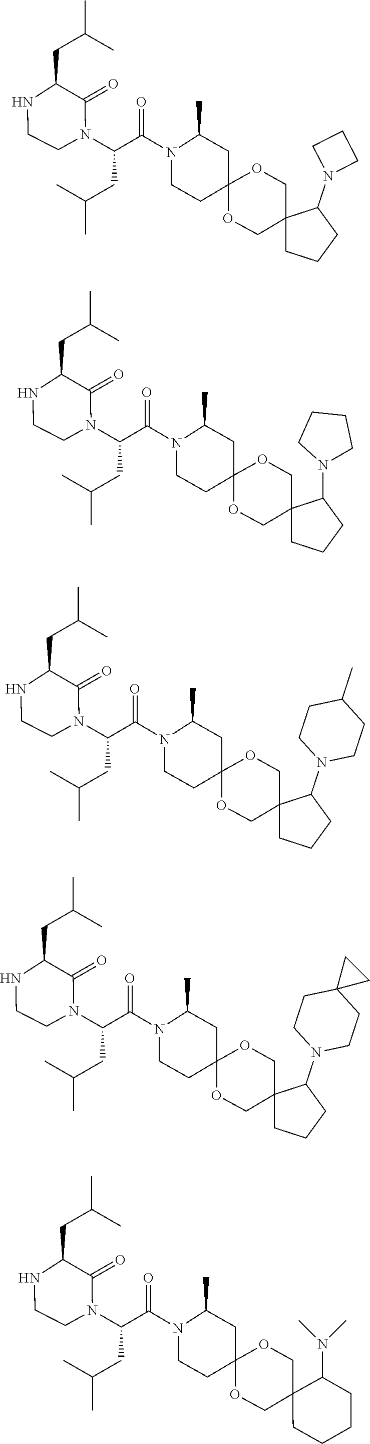 [SMILES: CC(C)C[C@@H]1NCCN([C@@H](CC(C)C)C(=O)N2CCC3(C[C@@H]2C)OCC2(CCCC2N2CCC2)CO3)C1=O ; CC(C)C[C@@H]1NCCN([C@@H](CC(C)C)C(=O)N2CCC3(C[C@@H]2C)OCC2(CCCC2N2CCCC2)CO3)C1=O ; CC(C)C[C@@H]1NCCN([C@@H](CC(C)C)C(=O)N2CCC3(C[C@@H]2C)OCC2(CCCC2N2CCC(C)CC2)CO3)C1=O ; CC(C)C[C@@H]1NCCN([C@@H](CC(C)C)C(=O)N2CCC3(C[C@@H]2C)OCC2(CCCC2N2CCC4(CC2)CC4)CO3)C1=O ; CC(C)C[C@@H]1NCCN([C@@H](CC(C)C)C(=O)N2CCC3(C[C@@H]2C)OCC2(CCCCC2N(C)C)CO3)C1=O]

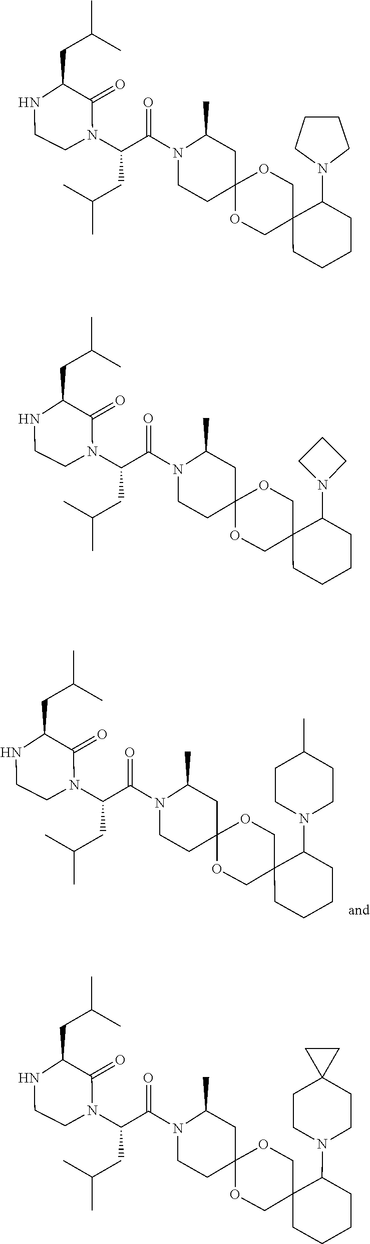 [SMILES: CC(C)C[C@@H]1NCCN([C@@H](CC(C)C)C(=O)N2CCC3(C[C@@H]2C)OCC2(CCCCC2N2CCCC2)CO3)C1=O ; CC(C)C[C@@H]1NCCN([C@@H](CC(C)C)C(=O)N2CCC3(C[C@@H]2C)OCC2(CCCCC2N2CCC2)CO3)C1=O ; CC(C)C[C@@H]1NCCN([C@@H](CC(C)C)C(=O)N2CCC3(C[C@@H]2C)OCC2(CCCCC2N2CCC(C)CC2)CO3)C1=O ; CC(C)C[C@@H]1NCCN([C@@H](CC(C)C)C(=O)N2CCC3(C[C@@H]2C)OCC2(CCCCC2N2CCC4(CC2)CC4)CO3)C1=O]

As used herein, the term “pharmaceutically acceptable salt” refers to those salts of the compounds formed by the process of the present application which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the application, or separately by reacting the free base or acid function with a suitable acid or base.

Examples of pharmaceutically acceptable salts include, but are not limited to, nontoxic acid addition salts: salts formed with inorganic acids such as hydrochloric acid, hydrobromic acid, phosphoric acid, sulfuric acid and perchloric acid, or with organic acids such as acetic acid, maleic acid, tartaric acid, citric acid, succinic acid or malonic acid. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters of the compounds formed by the process of the present application which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein, refers to those prodrugs of the compounds formed by the process of the present application which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present application. “Prodrug”, as used herein, means a compound which is convertible in vivo by metabolic means (e.g., by hydrolysis) to afford any compound delineated by the formulae of the instant application. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38(1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

This application also encompasses pharmaceutical compositions containing, and methods of treating disorders through administering, pharmaceutically acceptable prodrugs of compounds of the application. For example, compounds of the application having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the application. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxy carbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

The application also provides for a pharmaceutical composition comprising a therapeutically effective amount of a compound of the application, or an enantiomer, diastereomer, stereoisomer, or pharmaceutically acceptable salt thereof, and a pharmaceutically acceptable carrier.

In another aspect, the application provides a method of synthesizing a compound disclosed herein.

The synthesis of the compounds of the application can be found herein and in the Examples below.

Other embodiments are a method of making a compound of any of the formulae herein using any one, or combination of, reactions delineated herein. The method can include the use of one or more intermediates or chemical reagents delineated herein.

Another aspect is an isotopically labeled compound of any of the formulae delineated herein. Such compounds have one or more isotope atoms which may or may not be radioactive (e.g., ³H, ²H, ¹⁴C, ¹³C, ¹⁸F, ³⁵S, ³²P, ¹²⁵I, and ¹³¹I) introduced into the compound. Such compounds are useful for drug metabolism studies and diagnostics, as well as therapeutic applications.

A compound of the application can be prepared as a pharmaceutically acceptable acid addition salt by reacting the free base form of the compound with a pharmaceutically acceptable inorganic or organic acid. Alternatively, a pharmaceutically acceptable base addition salt of a compound of the application can be prepared by reacting the free acid form of the compound with a pharmaceutically acceptable inorganic or organic base.

Alternatively, the salt forms of the compounds of the application can be prepared using salts of the starting materials or intermediates.

The free acid or free base forms of the compounds of the application can be prepared from the corresponding base addition salt or acid addition salt from, respectively. For example, a compound of the application in an acid addition salt form can be converted to the corresponding free base by treating with a suitable base (e.g., ammonium hydroxide solution, sodium hydroxide, and the like). A compound of the application in a base addition salt form can be converted to the corresponding free acid by treating with a suitable acid (e.g., hydrochloric acid, etc.).

The compounds of the present invention may be used in the form of pharmaceutically-acceptable salts derived from inorganic or organic acids. By “pharmaceutically-acceptable salt” is meant those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically-acceptable salts are well-known in the art. The salts may be prepared in situ during the final isolation and purification of the compounds of the invention or separately by reacting a free base function with a suitable acid.

Representative acid addition salts include, but are not limited to trifluoroacetic acid (TFA), formate, acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethanesulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically-acceptable basic addition salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

Prodrugs of the compounds of the application can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the application with a suitable carbamylating agent (e.g., 1,1-acyloxyalkylcarbanochloridate, para-nitrophenyl carbonate, or the like).

Protected derivatives of the compounds of the application can be made by means known to those of ordinary skill in the art. A detailed description of techniques applicable to the creation of protecting groups and their removal include, but are not limited to, those illustrated in T. W. Greene, “Protecting Groups in Organic Chemistry”, 3rd edition, John Wiley and Sons, Inc., 1999.

Compounds of the present application can be conveniently prepared or formed during the process of the application, as solvates (e.g., hydrates). Hydrates of compounds of the present application can be conveniently prepared by recrystallization from an aqueous/organic solvent mixture, using organic solvents such as dioxin, tetrahydrofuran or methanol.

Acids and bases useful in the methods herein are known in the art. Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Bases are any basic chemical, which can be inorganic (e.g., sodium bicarbonate, potassium hydroxide) or organic (e.g., triethylamine, pyridine) in nature. Bases are useful in either catalytic or stoichiometric amounts to facilitate chemical reactions.

Combinations of substituents and variables envisioned by this application are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

When any variable (e.g., R₁₄) occurs more than one time in any constituent or formula for a compound, its definition at each occurrence is independent of its definition at every other occurrence. Thus, for example, if a group is shown to be substituted with one or more R₁₄ moieties, then R₁₄ at each occurrence is selected independently from the definition of R₁₄. Also, combinations of substituents and/or variables are permissible, but only if such combinations result in stable compounds within a designated atom's normal valency.

In addition, some of the compounds of this application have one or more double bonds, or one or more asymmetric centers. Such compounds can occur as racemates, racemic mixtures, single enantiomers, individual diastereomers, diastereomeric mixtures, and cis- or trans- or E- or Z—double isomeric forms, and other stereoisomeric forms that may be defined, in terms of absolute stereochemistry, as (R)- or (S)-, or as (D)- or (L)- for amino acids. When the compounds described herein contain olefinic double bonds or other centers of geometric asymmetry or even E or Z isomerism across several bonds and/or rings, and unless specified otherwise, it is intended that the compounds include both E and Z geometric isomers. The configuration of any carbon-carbon double bond appearing herein is selected for convenience only and is not intended to designate a particular configuration unless the text so states; thus a carbon-carbon double bond depicted arbitrarily herein as trans may be cis, trans, or a mixture of the two in any proportion. All such isomeric forms of such compounds are expressly included in the present application.

Optical isomers may be prepared from their respective optically active precursors by the procedures described herein, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981).

“Isomerism” means compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

A carbon atom bonded to four non-identical substituents is termed a “chiral center”.

“Chiral isomer” means a compound with at least one chiral center. Compounds with more than one chiral center may exist either as an individual diastereomer or as a mixture of diastereomers, termed “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds and/or other rigid structures such as a ring or polycyclic system. These configurations are differentiated in their names by the prefixes cis and trans, or Z and E, which indicate that the groups are on the same or opposite side of the double bond and/or other rigid structures such as a ring or a polycyclic system in the molecule according to the Cahn-Ingold-Prelog rules.

Furthermore, the structures and other compounds discussed in this application include all atropic isomers thereof “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques; it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose. Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-enamine. The compounds of this application may also be represented in multiple tautomeric forms, in such instances, the application expressly includes all tautomeric forms of the compounds described herein (e.g., alkylation of a ring system may result in alkylation at multiple sites, the application expressly includes all such reaction products).

In the present application, the structural formula of the compound represents a certain isomer for convenience in some cases, but the present application includes all isomers, such as geometrical isomers, optical isomers based on an asymmetrical carbon, stereoisomers, tautomers, and the like. In the present specification, the structural formula of the compound represents a certain isomer for convenience in some cases, but the present application includes all isomers, such as geometrical isomers, optical isomers based on an asymmetrical carbon, stereoisomers, tautomers, and the like.

Compounds of the present invention can exist as stereoisomers wherein asymmetric or chiral centers are present. These compounds are designated by the symbols “R” or “S”, depending on the configuration of substituents around the chiral carbon atom. The present invention contemplates various stereoisomers and mixtures thereof. Stereoisomers include enantiomers and diastereomers, and mixtures of enantiomers or diastereomers. Individual stereoisomers of compounds of the present invention can be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns.

Geometric isomers can also exist in the compounds of the present invention. The present invention contemplates the various geometric isomers and mixtures thereof resulting from the arrangement of substituents around a carbon-carbon double bond or arrangement of substituents around a carbocyclic or heterocyclic ring.

Compounds of the present invention can also exist as racemates which is given the descriptor “rac”. The term racemate, as used herein, means an equimolar mixture of a pair of enantiomers. A racemate is usually formed when synthesis results in the generation of a stereocenter. As used herein, the term racemic mixture means racemate. Compounds of the present invention can also exist as diastereomeric meso forms which is given the descriptor “rel”. The term diastereomeric meso form as used herein means achiral forms with a pseudostereogenic C-atom, which is given the descriptor “r” or “s”, respectively.

Additionally, the compounds of the present application, for example, the salts of the compounds, can exist in either hydrated or unhydrated (the anhydrous) form or as solvates with other solvent molecules. Non-limiting examples of hydrates include monohydrates, dihydrates, etc. Non-limiting examples of solvates include ethanol solvates, acetone solvates, etc.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

It should be appreciated that solvates and hydrates of the compound according to formula (I) or formula (Ia) are also within the scope of the present application. Methods of solvation are generally known in the art.

A further embodiment of the present invention may also include compounds which are identical to the compounds of formula (I) or formula (Ia) except that one or more atoms are replaced by an atom having an atomic mass number or mass different from the atomic mass number or mass usually found in nature, e.g. compounds enriched in ²H (D), ³H, ¹³C, ¹²⁷I, etc. These isotopic analogs and their pharmaceutical salts and formulations are considered useful agents in therapy and/or diagnosis, for example, but not limited to, where a fine-tuning of in vivo half-life time could lead to an optimized dosage regimen.

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired bridged macrocyclic products of the present application. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds of this application may be modified by appending various functionalities via any synthetic means delineated herein to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

The compounds of the application are defined herein by their chemical structures and/or chemical names. Where a compound is referred to by both a chemical structure and a chemical name, and the chemical structure and chemical name conflict, the chemical structure is determinative of the compound's identity.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

### Methods of Synthesizing the Compounds

The compounds of the invention may be prepared by the exemplary processes described in the following reaction schemes or by the processes described in the examples below.

Exemplary reagents and procedures for these reactions appear hereinafter. Starting materials can be purchased or readily prepared by one of ordinary skill in the art.

Compounds of the present application can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; and Greene, T. W., Wuts, P. G. M., Protective Groups in Organic Synthesis, 3^{rd} edition, John Wiley & Sons: New York, 1999, incorporated by reference herein, are useful and recognized reference textbooks of organic synthesis known to those in the art. The following descriptions of synthetic methods are designed to illustrate, but not to limit, general procedures for the preparation of compounds of the present application. The processes generally provide the desired final compound at or near the end of the overall process, although it may be desirable in certain instances to further convert the compound to a pharmaceutically acceptable salt, ester or prodrug thereof. Suitable synthetic routes are depicted in the schemes below.

Those skilled in the art will recognize if a stereocenter exists in the compounds disclosed herein. Accordingly, the present application includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds of the present application can be prepared in a number of ways well known to those skilled in the art of organic synthesis. By way of example, compounds of the present application can be synthesized using the methods described below, together with synthetic methods known in the art of synthetic organic chemistry, or variations thereon as appreciated by those skilled in the art. Preferred methods include but are not limited to those methods described below.

Compounds of the present application can be synthesized by following the steps outlined in General Scheme 1 (Method A), General Scheme 2 (Method B1 and Method B2) and General Scheme 3 (Method C) which comprise different sequences of assembling intermediates. Starting materials are either commercially available or made by known procedures in the reported literature or as illustrated.

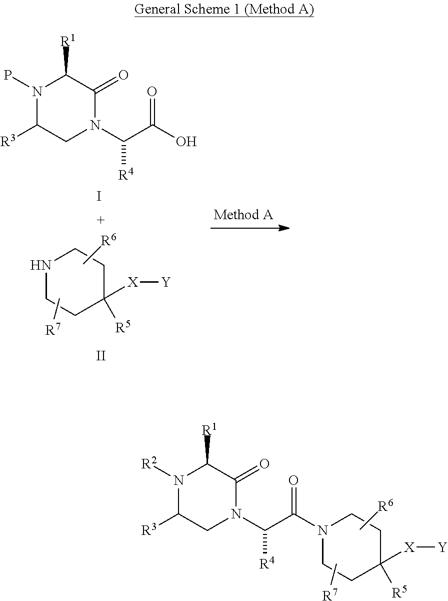

Method A: Using I where P is a suitable protecting group such as tBoc or nosyl, I and II are coupled using a dehydrating agent such as DCC or HATU in a suitable solvent such as DMF or NMP. The compounds where R² is H are obtained by deprotection under standard conditions. The compounds where is R² is C(O)R¹⁴, C(O)NR¹⁵R¹⁵ or C(O)OR¹⁵ are obtained by acylation of the secondary amine. The compounds where is R2 not the above are obtained by reductive amination of the secondary amine with the appropriate aldehyde or ketone.

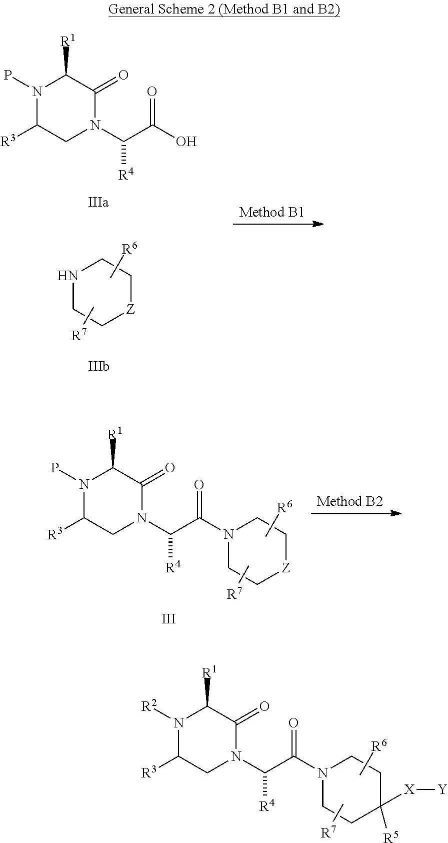

Method B1: Using the appropriate precursor IIIa and IIIb, III is prepared by amide coupling using a dehydrating agent such as DCC or HATU in a suitable solvent such as DMF or NMP.

Method B2: Z can be elaborated into the desired functional group using reaction sequences described in Table X. In cases where compound of the invention has R²=H, the starting material of Method B will have P as a protecting group, such as t-Boc or Nosyl. The compounds of the invention are then obtained by deprotection under standard conditions. The compounds where is R² is C(O)R¹⁴, C(O)NR¹⁵R¹⁵ or C(O)OR¹⁵ are obtained by acylation of the secondary amine at this point. The compounds where is R² is not the above are obtained by reductive amination of the secondary amine with the appropriate aldehyde or ketone.

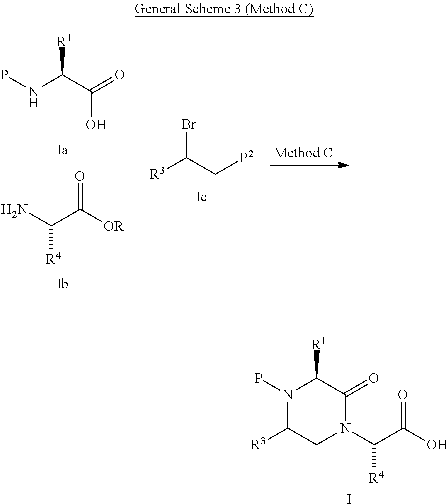

Method C: I may be prepared following the sequence described in Method C. Using the appropriate Ia bearing a protecting group P and Ib bearing a short alkyl group R, the coupling is performed by using a dehydrating agent such as DCC or HATU in a suitable solvent such as DMF or NMP. The resulting dipeptide ester is reacted with Ic. When P2 is Br, Ic is reacted in a suitable solvent such as DMF or DMSO in the presence of a base such as potassium carbonate or cesium carbonate to yield a short-chain ester derivative of I. When P2 is a protected alcohol such as OTHP or OTBDMS, the short chain ester of I is obtained by first reacting Ic with the dipeptide ester in a suitable solvent such as DMF or DMSO in the presence of a base such as potassium carbonate or cesium carbonate, followed by alcohol deprotection, followed by alcohol activation and coupling using methods like the Mitsunobu reaction or the formation of a mesylate and base-catalyzed cyclization. I is finally obtained by hydrolysis using a base such as sodium hydroxide or potassium carbonate, in a suitable solvent such as water or a water-THF mixture.

### Pharmaceutical Compositions

In a further aspect the present invention provides a pharmaceutical composition comprising a compound of formula (I) or formula (Ia) according to the invention and a pharmaceutically acceptable diluent, excipient or carrier.

In one embodiment the pharmaceutical composition further comprises another pharmaceutical active agent.

In one embodiment, the invention provides a pharmaceutical composition comprising a compound of formula (I) or formula (Ia) according to the invention and a pharmaceutically acceptable diluent, excipient or carrier, wherein said compound of formula (I) or formula (Ia) is present in a therapeutically effective amount.

The expression “effective amount” or “therapeutically effective amount” as used herein refers to an amount capable of invoking one or more of the following effects in a subject receiving the combination of the present invention: (i) inhibition or arrest of tumor growth, including, reducing the rate of tumor growth or causing complete growth arrest; (ii) reduction in the number of tumor cells; (iii) reduction in tumor size; (iv) reduction in tumor number; (v) inhibition of metastasis (i.e., reduction, slowing down or complete stopping) of tumor cell infiltration into peripheral organs; (vi) enhancement of antitumor immune response, which may, but does not have to, result in the regression or elimination of the tumor; (vii) relief, to some extent, of one or more symptoms associated with cancer; (viii) increase in progression-free survival (PFS) and/or; overall survival (OS) of the subject receiving the combination.

The compounds of the present invention may, in accordance with the invention, be administered in single or divided doses by oral, parenteral, inhalatory, rectal or topical administration including cutaneous, ophthalmic, mucosal scalp, sublingual, buccal and intranasal routes of administration; further, the compounds provided by the invention may be formulated to be used for the treatment of leukocyte populations in vivo, ex vivo and in vitro.

When the compounds of the present invention are to be administered e.g. by the oral route, they may be administered as medicaments in the form of pharmaceutical compositions which contain them in association with a pharmaceutically acceptable diluent, excipient or carrier material. Thus the present invention also provides a pharmaceutical composition comprising the compounds according to the invention as described supra and one or more pharmaceutically acceptable diluent, excipient or carrier. The pharmaceutical compositions can be prepared in a conventional manner and finished dosage forms can be solid dosage forms, for example, tablets, dragees, capsules, and the like, or liquid dosage forms, for example solutions, suspensions, emulsions and the like. Pharmaceutically acceptable diluent, excipient or carrier include sterile aqueous solutions or dispersions and sterile powders for the extemporaneous preparation of sterile injectable solutions or dispersion. The use of such media and agents for pharmaceutically active substances is known in the art.

In one embodiment, the invention provides a pharmaceutical composition comprising a compound of formula (I) or formula (Ia) according to the invention and at least one pharmaceutically acceptable diluent, excipient or carrier, wherein the composition is a tablet or a capsule, preferably a tablet.

The amount of the compounds of the invention to be administered will vary depending upon factors such as the particular compound, disease condition and its severity, according to the particular circumstances surrounding the case, including, e.g., the specific compound being administered, the route of administration, the condition being treated, the target area being treated, and the subject or host being treated

In another aspect, the application provides a pharmaceutical composition comprising a therapeutically effective amount of a compound of the present application or an enantiomer, diastereomer, or stereoisomer thereof, or pharmaceutically acceptable salt, hydrate, solvate, or prodrug thereof, and a pharmaceutically acceptable carrier.

Compounds of the application can be administered as pharmaceutical compositions by any conventional route, in particular enterally, e.g., orally, e.g., in the form of tablets or capsules, or parenterally, e.g., in the form of injectable solutions or suspensions, or topically, e.g., in the form of lotions, gels, ointments or creams, or in a nasal or suppository form. Pharmaceutical compositions comprising a compound of the present application in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present application with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

The pharmaceutical compositions of the present application comprise a therapeutically effective amount of a compound of the present application formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylenepolyoxy propylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes, oils such as peanut oil, cottonseed oil; safflower oil; sesame oil; olive oil; corn oil and soybean oil; glycols such a propylene glycol or polyethylene glycol; esters such as ethyl oleate and ethyl laurate, agar; buffering agents such as magnesium hydroxide and aluminum hydroxide; alginic acid; pyrogen-free water, isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this application can be administered to humans and other animals orally, rectally, parenterally, intracisternally, intravaginally, intraperitoneally, topically (as by powders, ointments, or drops), buccally, or as an oral or nasal spray.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous, or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this application with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

Dosage forms for topical or transdermal administration of a compound of this application include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this application.

The ointments, pastes, creams and gels may contain, in addition to an active compound of this application, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this application, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Compounds and compositions of the application can be administered in therapeutically effective amounts in a combinational therapy with one or more therapeutic agents (pharmaceutical combinations) or modalities, e.g., an anti-proliferative, anti-cancer, immunomodulatory or anti-inflammatory agent. Where the compounds of the application are administered in conjunction with other therapies, dosages of the co-administered compounds will of course vary depending on the type of co-drug employed, on the specific drug employed, on the condition being treated and so forth. Compounds and compositions of the application can be administered in therapeutically effective amounts in a combinational therapy with one or more therapeutic agents (pharmaceutical combinations) or modalities, e.g., anti-proliferative, anti-cancer, immunomodulatory or anti-inflammatory agent, and/or non-drug therapies, etc. For example, synergistic effects can occur with anti-proliferative, anti-cancer, immunomodulatory or anti-inflammatory substances. Where the compounds of the application are administered in conjunction with other therapies, dosages of the co-administered compounds will of course vary depending on the type of co-drug employed, on the specific drug employed, on the condition being treated and so forth.

Combination therapy includes the administration of the subject compounds in further combination with one or more other biologically active ingredients. For instance, the compounds of the application can be used in combination with other pharmaceutically active compounds, preferably compounds that are able to enhance the effect of the compounds of the application. The compounds of the application can be administered simultaneously (as a single preparation or separate preparation), in temporal proximity, or sequentially to the other drug therapy or treatment modality. In general, a combination therapy envisions administration of two or more drugs during a single cycle or course of therapy.

In another aspect of the application, the compounds may be administered in combination with one or more separate pharmaceutical agents, e.g., a chemotherapeutic agent, an immunotherapeutic agent, or an adjunctive therapeutic agent.

### Methods of Treatment

The compounds according to the invention as described supra have preventive and therapeutic utility in human and veterinary diseases.

Thus, in a further aspect the present invention provides the use of the compounds as described herein and the use of the pharmaceutical composition described herein for preventive and/or therapeutic purposes.

In one embodiment of the present invention, the compounds according to the invention as described herein or the pharmaceutical composition as described herein may be used as a medicament, preferably for use in human medicine and/or veterinarian medicine. Accordingly the present invention provides the compounds according to the invention as described herein or a pharmaceutical composition as described herein, for use as a medicament.

In another embodiment, the compounds according to the invention as described herein or the pharmaceutical composition as described herein may be used in a method for preventing or treating cancer in a subject.

Also provided is the use of the compounds according to the invention as described herein or the pharmaceutical composition as described herein for the manufacture of a medicament for the prevention or treatment of cancer in a subject.

Also provided is the use of the compounds according to the invention as described herein or the pharmaceutical composition as described herein for the prevention or treatment of cancer in a subject.

Also provided is a method for the prevention or treatment of cancer in a subject, comprising administering to said subject a therapeutically effective amount of the compounds according to the invention as described herein or the pharmaceutical composition as described herein.

The terms “treatment”/“treating” as used herein includes: (1) delaying the appearance of clinical symptoms of the state, disorder or condition developing in an animal, particularly a mammal and especially a human, that may be afflicted with or predisposed to the state, disorder or condition but does not yet experience or display clinical or subclinical symptoms of the state, disorder or condition; (2) inhibiting the state, disorder or condition (e.g. arresting, reducing or delaying the development of the disease, or a relapse thereof in case of maintenance treatment, of at least one clinical or subclinical symptom thereof); and/or (3) relieving the condition (i.e. causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

Preventive treatments comprise prophylactic treatments. In preventive applications, the pharmaceutical combination of the invention is administered to a subject suspected of having, or at risk for developing cancer. In therapeutic applications, the pharmaceutical combination is administered to a subject such as a patient already suffering from cancer, in an amount sufficient to cure or at least partially arrest the symptoms of the disease. Amounts effective for this use will depend on the severity and course of the disease, previous therapy, the subject's health status and response to the drugs, and the judgment of the treating physician.

In the case wherein the subject's condition does not improve, the pharmaceutical combination of the invention may be administered chronically, which is, for an extended period of time, including throughout the duration of the subject's life in order to ameliorate or otherwise control or limit the symptoms of the subject's disease or condition.

In the case wherein the subject's status does improve, the pharmaceutical combination may be administered continuously; alternatively, the dose of drugs being administered may be temporarily reduced or temporarily suspended for a certain length of time (i.e., a “drug holiday”). Once improvement of the patient's condition has occurred, a maintenance dose of the pharmaceutical combination of the invention is administered if necessary. Subsequently, the dosage or the frequency of administration, or both, is optionally reduced, as a function of the symptoms, to a level at which the improved disease is retained.

When provided preventively, the compound(s) are provided in advance of established disease. The preventive administration of a compound of the present invention serves to prevent or attenuate the evolution of disease. The therapeutic administration of a compound of the present invention serves to attenuate established disease. Thus, in accordance with the invention, a compound of the present invention can be administered either prior to the onset of disease or during the course of disease.

In one embodiment of the invention, there is provided the compounds according to the invention as described supra or the pharmaceutical composition as described supra, for use in a method for the prevention or treatment of cancer in a subject. Preferably the cancer is selected from the group consisting of head cancer and neck cancer.

### P300

Dysregulation of the cellular transcription machinery is a fundamental feature of cancer. E1A binding protein (p300) and CREB binding protein (CBP) are two closely related paralog transcriptional co-activators involved in the expression of oncogenic drivers in cancer cells (Attar and Kurdistani in Cold Spring Harbor Perspectives in Medicine 7:a026534 (2017)).

P300/CBP interact through their conserved domains with hundreds of proteins; can act synergistically or antagonistically; and modulate downstream biological processes in a highly context-dependent manner to promote either apoptosis or cell proliferation (Bedford and co-workers in Epigenetics 5(1): 9 (2010); Goodman and Smolik in Genes & Development 14(13):1553 (2000); Dancy and Cole in Chemical Reviews 115(6):2419 (2015)). These domains include the nuclear receptor interaction domain (RID), the cysteine/histidine regions CH1 (TAZ1) and CH3 (TAZ2), the CREB and MYB interaction domain (KIX), Bromodomain, the plant homeodomain (PHD), the histone acetyltransferase and/or lysine acetyltransferase domain (KAT/HAT), the ZZ type zinc finger domain (ZZ), and the interferon response binding domain (IBiD (NCBD)).

The following examples (from Dancy and Cole in Chemical Reviews 115(6):2419 (2015)) demonstrate the context-dependency of gene expression regulation by p300/CBP. For instance, Hottiger and co-workers (in EMBO Journal 17, 3124 (1998)) showed that HIV gene expression could be upregulated by tumor-necrosis factor alpha through binding of the RelA subunit of NFκB to p300/CBP—CH1 but was repressed through interferon-alpha-mediated binding of STAT2 to the same motif. In other studies, p300/CBP mediated both induction and repression of antioxidant response genes, via AP-1 binding to the C-terminal region, respectively by p53-binding to CH1/CH3 and glucocorticoid receptor-binding to the NRID domain (Avantaggiati and co-workers in Cell 89:1175 (1997); Kamei and co-workers in Cell 85:403 (1996)). P53 binding to p300/CBP/CH3 and consequent induction of p53-dependent genes results in cell cycle arrest (e.g., as a consequence of genotoxic insults), but apoptosis is induced when overexpressed E2F-1 (a central protein in cell cycle regulation that can act through p53 as well) is bound to p300/CBP/CH3 (Goodman and Smolik in Genes & Development 14:1553 (2000); Lee and co-workers in Oncogene 16:2695 (1998)). Cyclic-AMP response is both induced and repressed by p300/CBP via CREB binding to the KIX domain, respectively S6 kinase pp90RSK binding to the CH3 domain (Nakajima and co-workers in Cell 86:465 (1996)).

Modulation of cancer-relevant pathways by p300/CBP include hormone-dependent androgen receptor signaling in prostate cancer (Culig in Journal of Cell Physiology 231(2):270 (2016)); the HIF-1 alpha/VEGF pathway in hypoxia-dependent tumor growth (Masoud and Li in Acta Pharmacologica Sinica B 5(5):378 (2015)); and the interaction with tumor suppressor p53 and HPV-E6 oncoprotein in HPV-positive carcinomas (Tornesello and co-workers in Cancers (Basel) 10(7)pii:E213 (2018)).

P300 and CBP also play an important role in hematopoiesis and control processes whose disruption can lead to the development of leukemias and lymphomas (Blobel in Blood 95(3):745 (2000); Dutta and co-workers in Molecular Genetics and Metabolism 119(1-2):37 (2016)).

Taken together, these studies highlight how indispensable p300/CBP is to many cellular signaling pathways and how p300 and CBP utilize their protein-protein interactions to determine how the cell responds to environmental stimuli. This makes CBP/p300 an ideal target for the development of novel cancer therapies (Di Martile and co-workers in Oncotarget 7(34):55789 (2016); Ali and co-workers in Chemical Reviews 118(3):1216 (2018)).

Exploitation of CBP/p300 protein-protein interactions for drug discovery has nonetheless proven difficult because of the inherent highly disordered nature of the protein structure (Wright and Dyson in Nature Reviews in Molecular and Cell Biology 16(1): 18-29 (2015)). Yet specific inhibitors have been developed against highly conserved, more ordered domains such as the HAT/KAT catalytic site, KIX and bromodomain (Breen and Mapp in Current Opinion in Chemical Biology 45:195-203 (2018); Dancy and Cole in Chemical Reviews 115(6):2419-2452 (2015)).

Without being bound by any particular theory, an extremely well-conserved p300/CBP domain that can be a suitable drug target is the transcriptional adaptor and zinc finger 1 CH1/TAZ1 domain, as highlighted by several publications showing e.g. that the interaction between p300/CBP-CH1/TAZ1 and HIF1-alpha as well as the interaction between HPV-E6/E7 and p300/CBP-CH1/TAZ1 in HPV-positive Cervical and Head-and-Neck cancer can potentially be exploited for the development of anticancer therapies (Wuchano Yuan and Giordano in Oncogene 21:2253-2260(2002); Breen and Mapp in Current Opinion in Chemical Biology 45:195-203 (2018); Lao and co-workers in PNAS 111(21):7531 (2014); Kushal and co-workers in PNAS 110(39):15602 (2013); Masoud and Li in Acta Pharmacologica Sinica B 5(5):378 (2015); Burslem and co-workers in Chemical Science 8(6):4188 (2017); Fera and co-workers in Biochemistry 51 (47):9524 (2012); Xie and co-workers in Oncogene 33(8):1037 (2014); Patel and co-workers in The EMBO Journal 18(18):5061 (1999); Bernat and co-workers in Oncogene 22(39):7871 (2003)).

In summary, reprogramming the transcriptional profile of cancer cells by modulation of p300/CBP activity—for example by targeting the CH1/TAZ1 domain—represents a novel and broadly applicable approach for the treatment of cancer.

Without being bound by any particular theory, compounds of the disclosure can inhibit or modify the activity of p300 by inhibiting or modifying the activity of any p300 domain. For example, compounds of the disclosure can inhibit or modify the activity of the CH1/TAZ1, CH2/TAZ2, RID, KIX, KAT/HAT, PHD, Bromodomain, ZZ or IBiD domains. Compounds of the disclosure can inhibit or modify the interaction of p300 with any one of its protein interaction partners, or combination of protein interaction partners, through the CH1/TAZ1, CH2/TAZ2, RID, KIX, IBiD or any other p300 protein-protein interaction domain. A non-limiting list of p300 interaction partners whose interaction with p300 can be affected by compounds of the disclosure includes transcription coactivator BCL3 (BCL3), beta-catenin, breast cancer 1, early onset (BRCA1), caudal type homeobox 2 (CDX2), CCAAT enhancer binding protein beta (CEBPB) and CCAAT enhancer binding protein epsilon (CEBPE), Cbp/p300 interacting transactivator with Glu/Asp rich carboxy-terminal domain 1 (CITED1), Cbp/p300 interacting transactivator with Glu/Asp rich carboxy-terminal domain 2 (CITED2), DEAD-box helicase 5 (DDX5), deltex E3 ubiquitin ligase 1 (DTX1), EP300 interacting inhibitor of differentiation 1 (EID1), ELK1, ETS transcription factor (ELK1), estrogen receptor 1 (ESR1), flap structure-specific endonuclease 1 (FEN1), G protein pathway suppressor 2 (GPS2), hypoxia inducible factor 1 subunit alpha (HIF1A), HNF1 homeobox A (HNF1A), heterogeneous nuclear ribonucleoprotein U (HNRPU), inhibitor of growth family member 4 (ING4), inhibitor of growth family member 5 (ING5), interferon regulatory factor 2 (IRF2), lymphoid enhancer binding factor 1 (LEF1), MAF bZIP transcription factor (MAF), mastermind like transcriptional coactivator 1 (MAML1), myocyte enhancer factor 2C (MEF2C), myocyte enhancer factor 2D (MEF2D), MYB proto-oncogene like 2 (MYBL2), MDM2 proto-oncogene (Mdm2), myogenic differentiation 1 (MyoD), myocyte enhancer factor 2A (MEF2A), nuclear receptor coactivator 6 (NCOA6), nuclear factor of activated T cells 2 (NFATC2), neuronal PAS domain protein 2 (NPAS2), tumor protein p53 (P53), paired box 6 (PAX6), proliferating cell nuclear antigen (PCNA), prospero homeobox 1 (PROX1), prothymosin alpha (PTMA), peroxisome proliferator activated receptor alpha (PPARA), peroxisome proliferator activated receptor gamma (PPARG), RAR related orphan receptor A (RORA), RELA proto-oncogene, NF-kB subunit (RELA), SMAD family member 1 (SMAD1), SMAD family member 2 (SMAD2), MAD family member 7 (SMAD7), Smad nuclear interacting protein 1 (SNIP1), SS18, nBAF chromatin remodeling complex subunit (SS18), signal transducer and activator of transcription 3 (STAT3), signal transducer and activator of transcription 6 (STAT6), TAL bHLH transcription factor 1, erythroid differentiation factor (TALl), transcription factor 3 (TCF3), transcription factor AP-2 alpha (TFAP2A), trimethylguanosine synthase 1 (TGS1), transcriptional regulating factor 1 (TRERF1), tumor susceptibility 101 (TSG101), twist family bHLH transcription factor 1 (TWIST1), YY1 transcription factor (YY1) and early growth response 1 (Zif-268).

Without wishing to be bound by any particular theory, inhibiting or modifying the ability of p300 to interact with protein-protein interaction partners can inhibit or modify the ability of p300 or protein complexes comprising p300 to bind to DNA. For example, a compound of the disclosure can prevent p300 or a protein complex comprising p300 from binding to a target promoter, thereby preventing transcription of a target gene. A compound of the disclosure can prevent p300 or protein complexes comprising p300 from binding to a subset of all p300 target promoters, thereby altering the transcriptional profile of a cell, for example a cancer cell. Alternatively, or in addition, a compound of the disclosure can inhibit or modify the ability of p300 or a p300 protein complex to recruit one or more additional transcription factors, for example, transcription co-activators, to a promoter. Without limiting the possible pathways affected, a compound of the disclosure can alter the expression of genes involved in cell cycle progression, Wnt, Notch and Hedgehog signaling, DNA damage response, apoptosis, antioxidant response, Cyclic-AMP response, hormone-dependent androgen receptor signaling, hypoxia-dependent tumor growth, hematopoiesis or a combination thereof, thereby reducing the proliferation of or otherwise reducing the viability of cancer cells. For example, compounds of the disclosure can inhibit p300 interaction with CBP-HPVE6-p53, thereby rescuing p53 protein expression and acetylation and restoring the DNA damage response pathway in cervical cancer cells. Alternatively, or in addition, compounds of the disclosure can inhibit the formation of the p300/CBP-HIF1alpha protein complex, and reducing the transcription of growth factors and pro-proliferation genes such as vascular endothelial growth factor A (VEGF) in cancer cells. Alternatively, or in addition, compounds of the disclosure can disrupt p300-CH1/TAZ1 androgen receptor (AR) in castration resistant prostate cancers inhibiting the expression of AR target genes.

Without wishing to be bound by any particular theory, compounds of the disclosure can inhibit the activity of p300 in its regulation of oncogenic transcription factors that contribute to cancer progression.

Without wishing to be bound by any particular theory, compounds of the disclosure can act by inhibiting or modifying the acetyltransferase activity of the KAT/HAT domain.

An exemplary human p300 protein sequence can be found in NCBI NP_001420.2, the contents of which are hereby incorporated by reference in their entirety. An exemplary human p300 protein comprises a sequence of:

| (SEQ ID NO: 1) | |
|---|---|
| 1 | maenvvepgp psakrpklss palsasasdg tdfgslfdle hdlpdelins telgltnggd |
| 61 | inqlqtslgm vqdaaskhkq lsellrsgss pnlnmgvggp gqvmasqaqq sspglglins |
| 121 | mvkspmtqag ltspnmgmgt sgpnqgptqs tgmmnspvnq pamgmntgmn agmnpgmlaa |
| 181 | gngqgimpnq vmngsigagr grqnmqypnp gmgsagnllt eplqqgspqm ggqtglrgpq |
| 241 | plkmgmmnnp npygspytqn pgqqigasgl glqiqtktvl snnlspfamd kkavpgggmp |
| 301 | nmgqqpapqv qqpglvtpva qgmgsgahta dpekrkliqq qlvlllhahk cqrreqange |
| 361 | vrqcnlphcr tmknvlnhmt hcqsgkscqv ahcassrqii shwknctrhd cpvclplkna |
| 421 | gdkrnqqpil tgapvglgnp sslgvgqqsa pnlstvsqid pssierayaa lglpyqvnqm |
| 481 | ptqpqvqakn qqnqqpgqsp qgmrpmsnms aspmgvnggv gvqtpsllsd smlhsainsq |
| 541 | npmmsenasv pslgpmptaa qpsttgirkq wheditqdlr nhlvhklvqa ifptpdpaal |
| 601 | kdrrmenlva yarkvegdmy esannraeyy hllaekiyki qkeleekrrt rlqkqnmlpn |
| 661 | aagmvpvsmn pgpnmgqpqp gmtsngplpd psmirgsvpn qmmpritpqs glnqfgqmsm |
| 721 | aqppivprqt pplqhhgqla qpgalnppmg ygprmqqpsn qgqflpqtqf psqgmnvtni |
| 781 | plapssgqap vsqaqmssss cpvnspimpp gsqgshihcp qlpqpalhqn spspvpsrtp |
| 841 | tphhtppsig aqqppattip apvptppamp pgpqsqalhp pprqtptppt tqlpqqvqps |
| 901 | lpaapsadqp qqqprsqqst aasvptptap llppqpatpl sqpavsiegq vsnppstsst |
| 961 | evnsqaiaek qpsqevkmea kmevdqpepa dtqpedises kvedckmest eteerstelk |
| 1021 | teikeeedqp stsatqsspa pgqskkkifk peelrqalmp tlealyrqdp eslpfrqpvd |
| 1081 | pqllgipdyf divkspmdls tikrkldtgq yqepwqyvdd iwlmfnnawl ynrktsrvyk |
| 1141 | ycsklsevfe qeidpvmqsl gyccgrklef spqtlccygk qlctiprdat yysyqnryhf |
| 1201 | cekcfneiqg esvslgddps qpqttinkeq fskrkndtld pelfvectec grkmhqicvl |
| 1261 | hheiiwpagf vcdgclkksa rtrkenkfsa krlpstrlgt flenrvndfl rrqnhpesge |
| 1321 | vtvrvvhasd ktvevkpgmk arfvdsgema esfpyrtkal fafeeidgvd lcffgmhvqe |
| 1381 | ygsdcpppnq rrvyisylds vhffrpkclr tavyheilig yleyvkklgy ttghiwacpp |
| 1441 | segddyifhc hppdqkipkp krlqewykkm ldkavseriv hdykdifkqa tedrltsake |
| 1501 | lpyfegdfwp nvleesikel eqeeeerkre entsnestdv tkgdsknakk knnkktsknk |
| 1561 | sslsrgnkkk pgmpnvsndl sqklyatmek hkevffvirl iagpaanslp pivdpdplip |
| 1621 | cdlmdgrdaf ltlardkhle fsslrraqws tmcmlvelht qsqdrfvytc neckhhvetr |
| 1681 | whctvcedyd lcitcyntkn hdhkmeklgl glddesnnqq aaatqspgds rrlsiqrciq |
| 1741 | slvhacqcrn ancslpscqk mkrvvqhtkg ckrktnggcp ickqlialcc yhakhcqenk |
| 1801 | cpvpfclnik qklrqqqlqh rlqqaqmlrr rmasmqrtgv vgqqqglpsp tpatpttptg |
| 1861 | qqpttpqtpq ptsqpqptpp nsmppylprt qaagpvsqgk aagqvtpptp pqtaqpplpg |
| 1921 | pppaavemam qiqraaetqr qmahvqifqr piqhqmppmt pmapmgmnpp pmtrgpsghl |
| 1981 | epgmgptgmq qqppwsqggl pqpqqlqsgm prpammsvaq hgqplnmapq pglgqvgisp |
| 2041 | lkpgtvsqqa lqnllrtlrs pssplqqqqv lsilhanpql laafikqraa kyansnpqpi |
| 2101 | pgqpgmpqgq pglqpptmpg qqgvhsnpam qnmnpmqagv qraglpqqqp qqqlqppmgg |
| 2161 | mspqaqqmnm nhntmpsqfr dilrrqqmmq qqqqqgagpg igpgmanhnq fqqpqgvgyp |
| 2221 | pqqqqrmqhh mqqmqqgnmg qigqlpqalg aeagaslqay qqrllqqqmg spvqpnpmsp |
| 2281 | qqhmlpnqaq sphlqgqqip nslsnqvrsp qpvpsprpqs qpphsspspr mqpqpsphhv |
| 2341 | spqtssphpg lvaaqanpme qghfaspdqn smlsqlasnp gmanlhgasa tdlglstdns |
| 2401 | dlnsnlsqst ldih. |

In some embodiments, a p300 protein comprises a protein having at least 85% identity to SEQ ID NO: 1, at least 90% identity to SEQ ID NO: 1, at least 95% identity to SEQ ID NO: 1, 50 at least 96% identity to SEQ ID NO: 1, at least 97% identity to SEQ ID NO: 1, at least 98% identity to SEQ ID NO: 1, at least 99% identity to SEQ ID NO: 1 or at least 99.8% identity to SEQ ID NO: 1. In some embodiments, a p300 protein is identical to a protein of SEQ ID NO: 1.

The CH1/TAZ domain corresponds approximately to amino acids 347-414 of SEQ ID NO: 1. The KIX domain corresponds approximately to amino acids 566-646 of SEQ ID NO: 1. The bromodomain corresponds approximately to amino acids 1051-1158 of SEQ ID NO: 1. The PHD domain corresponds approximately to amino acids 1243-1277 of SEQ ID NO: 1. The HAT/KAT domain corresponds approximately to amino acids 1306-1612 of SEQ ID NO: 1. The ZZ domain corresponds approximately to amino acids 1668-1708 of SEQ ID NO: 1. The TAZ2 domain corresponds approximately to amino acids 1729-1807 of SEQ ID NO: 1.

As used herein in the context of polypeptides, nucleic acids, and chemical compounds, the term “corresponding to”, designates the position/identity of a structural element, e.g., of an amino acid residue, a nucleotide residue, or a chemical moiety, in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190th amino acid in a particular amino acid chain but rather corresponds to the residue found at position 190 in the reference polypeptide; those of ordinary skill in the art readily appreciate how to identify “corresponding” amino acids (see. e.g., Benson et al. Nucl. Acids Res. (1 Jan. 2013) 41 (D1): D36-D42; Pearson et al. PNAS Vol. 85, pp. 2444-2448, April 1988). Those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

As used herein the term “domain” refers to a section or portion of a polypeptide. In some embodiments, a “domain” is associated with a particular structural and/or functional feature of the polypeptide so that, when the domain is physically separated from the rest of its parent polypeptide, it substantially or entirely retains the particular structural and/or functional feature. In some embodiments, a domain may include a portion of a polypeptide that, when separated from that (parent) polypeptide and linked with a different (recipient) polypeptide, substantially retains and/or imparts on the recipient polypeptide one or more structural and/or functional features that characterized it in the parent polypeptide. In some embodiments, a domain is a section of a polypeptide. In some such embodiments, a domain is characterized by a particular structural element (e.g., a particular amino acid sequence or sequence motif, oc-helix character, b-sheet character, coiled-coil character, random coil character), and/or by a particular functional feature (e.g., binding activity, enzymatic activity, folding activity, signaling activity). One of ordinary skill will appreciate that domain boundaries are typically determined experimentally or via sequence alignment, and may be approximate. In some embodiments, domain boundaries may vary by at least 1, at least 2, at least 3, at least 4, at least 5, at least 6, at least 10, at least 15 or at least 20 amino acids without affecting the in vivo function of the domain.

An exemplary nucleic acid sequence encoding a p300 protein comprises a sequence of:

| (SEQ ID NO: 2) | |
|---|---|
| 1 | gagaaggagg aggacagcgc cgaggaggaa gaggttgatg gcggcggcgg agctccgaga |
| 61 | gacctcggct gggcaggggc cggccgtggc gggccgggga ctgcgcctct agagccgcga |
| 121 | gttctcggga attcgccgca gcggacgcgc tcggcgaatt tgtgctcttg tgccctcctc |
| 181 | cgggcttggg cccaggcccg gcccctcgca cttgccctta ccttttctat cgagtccgca |
| 241 | tccctctcca gccactgcga cccggcgaag agaaaaagga acttccccca ccccctcggg |
| 301 | tgccgtcgga gccccccagc ccacccctgg gtgcggcgcg gggaccccgg gccgaagaag |
| 361 | agatttcctg aggattctgg ttttcctcgc ttgtatctcc gaaagaatta aaaatggccg |
| 421 | agaatgtggt ggaaccgggg ccgccttcag ccaagcggcc taaactctca tctccggccc |
| 481 | tctcggcgtc cgccagcgat ggcacagatt ttggctctct atttgacttg gagcacgact |
| 541 | taccagatga attaatcaac tctacagaat tgggactaac caatggtggt gatattaatc |
| 601 | agcttcagac aagtcttggc atggtacaag atgcagcttc taaacataaa cagctgtcag |
| 661 | aattgctgcg atctggtagt tcccctaacc tcaatatggg agttggtggc ccaggtcaag |
| 721 | tcatggccag ccaggcccaa cagagcagtc ctggattagg tttgataaat agcatggtca |
| 781 | aaagcccaat gacacaggca ggcttgactt ctcccaacat ggggatgggc actagtggac |
| 841 | caaatcaggg tcctacgcag tcaacaggta tgatgaacag tccagtaaat cagcctgcca |
| 901 | tgggaatgaa cacagggatg aatgcgggca tgaatcctgg aatgttggct gcaggcaatg |
| 961 | gacaagggat aatgcctaat caagtcatga acggttcaat tggagcaggc cgagggcgac |
| 1021 | agaatatgca gtacccaaac ccaggcatgg gaagtgctgg caacttactg actgagcctc |
| 1081 | ttcagcaggg ctctccccag atgggaggac aaacaggatt gagaggcccc cagcctctta |
| 1141 | agatgggaat gatgaacaac cccaatcctt atggttcacc atatactcag aatcctggac |
| 1201 | agcagattgg agccagtggc cttggtctcc agattcagac aaaaactgta ctatcaaata |
| 1261 | acttatctcc atttgctatg gacaaaaagg cagttcctgg tggaggaatg cccaacatgg |
| 1321 | gtcaacagcc agccccgcag gtccagcagc caggcctggt gactccagtt gcccaaggga |
| 1381 | tgggttctgg agcacataca gctgatccag agaagcgcaa gctcatccag cagcagcttg |
| 1441 | ttctcctttt gcatgctcac aagtgccagc gccgggaaca ggccaatggg gaagtgaggc |
| 1501 | agtgcaacct tccccactgt cgcacaatga agaatgtcct aaaccacatg acacactgcc |
| 1561 | agtcaggcaa gtcttgccaa gtggcacact gtgcatcttc tcgacaaatc atttcacact |
| 1621 | ggaagaattg tacaagacat gattgtcctg tgtgtctccc cctcaaaaat gctggtgata |
| 1681 | agagaaatca acagccaatt ttgactggag cacccgttgg acttggaaat cctagctctc |
| 1741 | taggggtggg tcaacagtct gcccccaacc taagcactgt tagtcagatt gatcccagct |
| 1801 | ccatagaaag agcctatgca gctcttggac taccctatca agtaaatcag atgccgacac |
| 1861 | aaccccaggt gcaagcaaag aaccagcaga atcagcagcc tgggcagtct ccccaaggca |
| 1921 | tgcggcccat gagcaacatg agtgctagtc ctatgggagt aaatggaggt gtaggagttc |
| 1981 | aaacgccgag tcttctttct gactcaatgt tgcattcagc cataaattct caaaacccaa |
| 2041 | tgatgagtga aaatgccagt gtgccctccc tgggtcctat gccaacagca gctcaaccat |
| 2101 | ccactactgg aattcggaaa cagtggcacg aagatattac tcaggatctt cgaaatcatc |
| 2161 | ttgttcacaa actcgtccaa gccatatttc ctacgccgga tcctgctgct ttaaaagaca |
| 2221 | gacggatgga aaacctagtt gcatatgctc ggaaagttga aggggacatg tatgaatctg |
| 2281 | caaacaatcg agcggaatac taccaccttc tagctgagaa aatctataag atccagaaag |
| 2341 | aactagaaga aaaacgaagg accagactac agaagcagaa catgctacca aatgctgcag |
| 2401 | gcatggttcc agtttccatg aatccagggc ctaacatggg acagccgcaa ccaggaatga |
| 2461 | cttctaatgg ccctctacct gacccaagta tgatccgtgg cagtgtgcca aaccagatga |
| 2521 | tgcctcgaat aactccacaa tctggtttga atcaatttgg ccagatgagc atggcccagc |
| 2581 | cccctattgt accccggcaa acccctcctc ttcagcacca tggacagttg gctcaacctg |
| 2641 | gagctctcaa cccgcctatg ggctatgggc ctcgtatgca acagccttcc aaccagggcc |
| 2701 | agttccttcc tcagactcag ttcccatcac agggaatgaa tgtaacaaat atccctttgg |
| 2761 | ctccgtccag cggtcaagct ccagtgtctc aagcacaaat gtctagttct tcctgcccgg |
| 2821 | tgaactctcc tataatgcct ccagggtctc aggggagcca cattcactgt ccccagcttc |
| 2881 | ctcaaccagc tcttcatcag aattcaccct cgcctgtacc tagtcgtacc cccacccctc |
| 2941 | accatactcc cccaagcata ggggctcagc agccaccagc aacaacaatt ccagcccctg |
| 3001 | ttcctacacc tcctgccatg ccacctgggc cacagtccca ggctctacat ccccctccaa |
| 3061 | ggcagacacc tacaccacca acaacacaac ttccccaaca agtgcagcct tcacttcctg |
| 3121 | ctgcaccttc tgctgaccag ccccagcagc agcctcgctc acagcagagc acagcagcgt |
| 3181 | ctgttcctac cccaacagca ccgctgcttc ctccgcagcc tgcaactcca ctttcccagc |
| 3241 | cagctgtaag cattgaagga caggtatcaa atcctccatc tactagtagc acagaagtga |
| 3301 | attctcaggc cattgctgag aagcagcctt cccaggaagt gaagatggag gccaaaatgg |
| 3361 | aagtggatca accagaacca gcagatactc agccggagga tatttcagag tctaaagtgg |
| 3421 | aagactgtaa aatggaatct accgaaacag aagagagaag cactgagtta aaaactgaaa |
| 3481 | taaaagagga ggaagaccag ccaagtactt cagctaccca gtcatctccg gctccaggac |
| 3541 | agtcaaagaa aaagattttc aaaccagaag aactacgaca ggcactgatg ccaactttgg |
| 3601 | aggcacttta ccgtcaggat ccagaatccc ttccctttcg tcaacctgtg gaccctcagc |
| 3661 | ttttaggaat ccctgattac tttgatattg tgaagagccc catggatctt tctaccatta |
| 3721 | agaggaagtt agacactgga cagtatcagg agccctggca gtatgtcgat gatatttggc |
| 3781 | ttatgttcaa taatgcctgg ttatataacc ggaaaacatc acgggtatac aaatactgct |
| 3841 | ccaagctctc tgaggtcttt gaacaagaaa ttgacccagt gatgcaaagc cttggatact |
| 3901 | gttgtggcag aaagttggag ttctctccac agacactgtg ttgctacggc aaacagttgt |
| 3961 | gcacaatacc tcgtgatgcc acttattaca gttaccagaa caggtatcat ttctgtgaga |
| 4021 | agtgtttcaa tgagatccaa ggggagagcg tttctttggg ggatgaccct tcccagcctc |
| 4081 | aaactacaat aaataaagaa caattttcca agagaaaaaa tgacacactg gatcctgaac |
| 4141 | tgtttgttga atgtacagag tgcggaagaa agatgcatca gatctgtgtc cttcaccatg |
| 4201 | agatcatctg gcctgctgga ttcgtctgtg atggctgttt aaagaaaagt gcacgaacta |
| 4261 | ggaaagaaaa taagttttct gctaaaaggt tgccatctac cagacttggc acctttctag |
| 4321 | agaatcgtgt gaatgacttt ctgaggcgac agaatcaccc tgagtcagga gaggtcactg |
| 4381 | ttagagtagt tcatgcttct gacaaaaccg tggaagtaaa accaggcatg aaagcaaggt |
| 4441 | ttgtggacag tggagagatg gcagaatcct ttccataccg aaccaaagcc ctctttgcct |
| 4501 | ttgaagaaat tgatggtgtt gacctgtgct tctttggcat gcatgttcaa gagtatggct |
| 4561 | ctgactgccc tccacccaac cagaggagag tatacatatc ttacctcgat agtgttcatt |
| 4621 | tcttccgtcc taaatgcttg aggactgcag tctatcatga aatcctaatt ggatatttag |
| 4681 | aatatgtcaa gaaattaggt tacacaacag ggcatatttg ggcatgtcca ccaagtgagg |
| 4741 | gagatgatta tatcttccat tgccatcctc ctgaccagaa gatacccaag cccaagcgac |
| 4801 | tgcaggaatg gtacaaaaaa atgcttgaca aggctgtatc agagcgtatt gtccatgact |
| 4861 | acaaggatat ttttaaacaa gctactgaag atagattaac aagtgcaaag gaattgcctt |
| 4921 | atttcgaggg tgatttctgg cccaatgttc tggaagaaag cattaaggaa ctggaacagg |
| 4981 | aggaagaaga gagaaaacga gaggaaaaca ccagcaatga aagcacagat gtgaccaagg |
| 5041 | gagacagcaa aaatgctaaa aagaagaata ataagaaaac cagcaaaaat aagagcagcc |
| 5101 | tgagtagggg caacaagaag aaacccggga tgcccaatgt atctaacgac ctctcacaga |
| 5161 | aactatatgc caccatggag aagcataaag aggtcttctt tgtgatccgc ctcattgctg |
| 5221 | gccctgctgc caactccctg cctcccattg ttgatcctga tcctctcatc ccctgcgatc |
| 5281 | tgatggatgg tcgggatgcg tttctcacgc tggcaaggga caagcacctg gagttctctt |
| 5341 | cactccgaag agcccagtgg tccaccatgt gcatgctggt ggagctgcac acgcagagcc |
| 5401 | aggaccgctt tgtctacacc tgcaatgaat gcaagcacca tgtggagaca cgctggcact |
| 5461 | gtactgtctg tgaggattat gacttgtgta tcacctgcta taacactaaa aaccatgacc |
| 5521 | acaaaatgga gaaactaggc cttggcttag atgatgagag caacaaccag caggctgcag |
| 5581 | ccacccagag cccaggcgat tctcgccgcc tgagtatcca gcgctgcatc cagtctctgg |
| 5641 | tccatgcttg ccagtgtcgg aatgccaatt gctcactgcc atcctgccag aagatgaagc |
| 5701 | gggttgtgca gcataccaag ggttgcaaac ggaaaaccaa tggcgggtgc cccatctgca |
| 5761 | agcagctcat tgccctctgc tgctaccatg ccaagcactg ccaggagaac aaatgcccgg |
| 5821 | tgccgttctg cctaaacatc aagcagaagc tccggcagca acagctgcag caccgactac |
| 5881 | agcaggccca aatgcttcgc aggaggatgg ccagcatgca gcggactggt gtggttgggc |
| 5941 | agcaacaggg cctcccttcc cccactcctg ccactccaac gacaccaact ggccaacagc |
| 6001 | caaccacccc gcagacgccc cagcccactt ctcagcctca gcctacccct cccaatagca |
| 6061 | tgccacccta cttgcccagg actcaagctg ctggccctgt gtcccagggt aaggcagcag |
| 6121 | gccaggtgac ccctccaacc cctcctcaga ctgctcagcc accccttcca gggcccccac |
| 6181 | ctgcagcagt ggaaatggca atgcagattc agagagcagc ggagacgcag cgccagatgg |
| 6241 | cccacgtgca aatttttcaa aggccaatcc aacaccagat gcccccgatg actcccatgg |
| 6301 | cccccatggg tatgaaccca cctcccatga ccagaggtcc cagtgggcat ttggagccag |
| 6361 | ggatgggacc gacagggatg cagcaacagc caccctggag ccaaggagga ttgcctcagc |
| 6421 | cccagcaact acagtctggg atgccaaggc cagccatgat gtcagtggcc cagcatggtc |
| 6481 | aacctttgaa catggctcca caaccaggat tgggccaggt aggtatcagc ccactcaaac |
| 6541 | caggcactgt gtctcaacaa gccttacaaa accttttgcg gactctcagg tctcccagct |
| 6601 | ctcccctgca gcagcaacag gtgcttagta tccttcacgc caacccccag ctgttggctg |
| 6661 | cattcatcaa gcagcgggct gccaagtatg ccaactctaa tccacaaccc atccctgggc |
| 6721 | agcctggcat gccccagggg cagccagggc tacagccacc taccatgcca ggtcagcagg |
| 6781 | gggtccactc caatccagcc atgcagaaca tgaatccaat gcaggcgggc gttcagaggg |
| 6841 | ctggcctgcc ccagcagcaa ccacagcagc aactccagcc acccatggga gggatgagcc |
| 6901 | cccaggctca gcagatgaac atgaaccaca acaccatgcc ttcacaattc cgagacatct |
| 6961 | tgagacgaca gcaaatgatg caacagcagc agcaacaggg agcagggcca ggaataggcc |
| 7021 | ctggaatggc caaccataac cagttccagc aaccccaagg agttggctac ccaccacagc |
| 7081 | agcagcagcg gatgcagcat cacatgcaac agatgcaaca aggaaatatg ggacagatag |
| 7141 | gccagcttcc ccaggccttg ggagcagagg caggtgccag tctacaggcc tatcagcagc |
| 7201 | gactccttca gcaacagatg gggtcccctg ttcagcccaa ccccatgagc ccccagcagc |
| 7261 | atatgctccc aaatcaggcc cagtccccac acctacaagg ccagcagatc cctaattctc |
| 7321 | tctccaatca agtgcgctct ccccagcctg tcccttctcc acggccacag tcccagcccc |
| 7381 | cccactccag tccttcccca aggatgcagc ctcagccttc tccacaccac gtttccccac |
| 7441 | agacaagttc cccacatcct ggactggtag ctgcccaggc caaccccatg gaacaagggc |
| 7501 | attttgccag cccggaccag aattcaatgc tttctcagct tgctagcaat ccaggcatgg |
| 7561 | caaacctcca tggtgcaagc gccacggacc tgggactcag caccgataac tcagacttga |
| 7621 | attcaaacct ctcacagagt acactagaca tacactagag acaccttgta gtattttggg |
| 7681 | agcaaaaaaa ttattttctc ttaacaagac tttttgtact gaaaacaatt tttttgaatc |
| 7741 | tttcgtagcc taaaagacaa ttttccttgg aacacataag aactgtgcag tagccgtttg |
| 7801 | tggtttaaag caaacatgca agatgaacct gagggatgat agaatacaaa gaatatattt |
| 7861 | ttgttatggc tggttaccac cagcctttct tcccctttgt gtgtgtggtt caagtgtgca |
| 7921 | ctgggaggag gctgaggcct gtgaagccaa acaatatgct cctgccttgc acctccaata |
| 7981 | ggttttatta ttttttttaa attaatgaac atatgtaata ttaatagtta ttatttactg |
| 8041 | gtgcagatgg ttgacatttt tccctatttt cctcacttta tggaagagtt aaaacatttc |
| 8101 | taaaccagag gacaaaaggg gttaatgtta ctttaaaatt acattctata tatatataaa |
| 8161 | tatatataaa tatatattaa aataccagtt ttttttctct gggtgcaaag atgttcattc |
| 8221 | ttttaaaaaa tgtttaaaaa aaaaaaaaaa ctgcctttct tcccctcaag tcaacttttg |
| 8281 | tgctccagaa aattttctat tctgtaagtc tgagcgtaaa acttcaagta ttaaaataat |
| 8341 | ttgtacatgt agagagaaaa atgacttttt caaaaatata caggggcagc tgccaaattg |
| 8401 | atgtattata tattgtggtt tctgtttctt gaaagaattt ttttcgttat ttttacatct |
| 8461 | aacaaagtaa aaaaattaaa aagagggtaa gaaacgattc cggtgggatg attttaacat |
| 8521 | gcaaaatgtc cctgggggtt tcttctttgc ttgctttctt cctccttacc ctacccccca |
| 8581 | ctcacacaca cacacacaca cacacacaca cacacacaca cacactttct ataaaacttg |
| 8641 | aaaatagcaa aaaccctcaa ctgttgtaaa tcatgcaatt aaagttgatt acttataaat |
| 8701 | atgaactttg gatcactgta tagactgtta aatttgattt cttattacct attgttaaat |
| 8761 | aaactgtgtg agacagaca. |

In some embodiments, a nucleic acid sequence encoding a p300 protein comprises a nucleic acid sequence encoding a protein having at least 85% identity to SEQ ID NO:1, at least 90% identity to SEQ ID NO:1, at least 95% identity to SEQ ID NO: 1, at least 96% identity to SEQ ID NO: 1, at least 97% identity to SEQ ID NO: 1, at least 98% identity to SEQ ID NO: 1, at least 99% identity to SEQ ID NO: 1 or at least 99.8% identity to SEQ ID NO: 1. In some embodiments, nucleic acid sequence encoding a p300 protein comprises a nucleic acid sequence encoding a protein identical to SEQ ID NO: 1. In some embodiments, a nucleic acid sequence encoding a p300 protein comprises a nucleic acid sequence e having at least 85% identity to SEQ ID NO: 2, at least 90% identity to SEQ ID NO: 2, at least 95% identity to SEQ ID NO: 2, at least 96% identity to SEQ ID NO: 2, at least 97% identity to SEQ ID NO: 2, at least 98% identity to SEQ ID NO: 2, at least 99% identity to SEQ ID NO: 2 or at least 99.8% identity to SEQ ID NO: 2. In some embodiments, a nucleic acid sequence encoding a p300 protein comprises a nucleic acid sequence identical to SEQ ID NO: 2 or a portion or subsequence thereof.

As used herein, the term “expression” of a nucleic acid sequence refers to the generation of any gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

As used herein, the term “nucleic acid” refers to a polymer of at least three nucleotides. In some embodiments, a nucleic acid comprises DNA. In some embodiments comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis).

### Methods of Treating Cancer

Cancer is a disease caused by the uncontrolled division of cells in the body. Abnormally dividing cancer cells can form a primary tumor, which can then invade nearby tissues, and spread throughout the body through the blood and lymphatic systems (metastatic cancers). Cancer can arise from many organs and cell types in the body, including but not limited to, cells of the lymphatic system, bone marrow, blood, brain and nervous system tissue, breast, cervix, ovary, colorectal cells, stomach and gastric cells, head and neck, kidney, liver, lung, oesophagus, pancreas, prostate and skin.

As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer.

In some embodiments, a tumor may be a disperse tumor or a liquid tumor. Liquid tumors can affect bone marrow, blood cells and the lymphatic system. Exemplary liquid tumors include leukemias and lymphomas. Types of lymphomas include, but are not limited to, Hodgkin lymphomas, non-Hodgkin lymphomas, B cell lymphomas, T-cell lymphomas, Burkitt's lymphomas, mantle cell lymphomas, small lymphocytic lymphomas, histiocytic lymphomas and primary mediastinal B cell lymphomas. Types of leukemias include, but are not limited to, acute myeloid leukemia, T cell leukemias, acute lymphoblastic leukemias and chronic myelogenous leukemias.

In some embodiments, a tumor may be a solid tumor. Exemplary solid tumors include, but are not limited to Carcinomas, Sarcomas, Myelomas, germ cell tumors, carcinoid tumors, neuroendocrine tumors and tumors of mixed type (a tumor which comprises multiple types of cancer cells). Carcinomas arise from epithelial tissues, either internal or external, such as cells of the gastrointestinal tract. Exemplary carcinomas include adenocarcinoma, which develops in an organ or gland, and squamous cell carcinoma, which originates in the squamous epithelium. Sarcomas are cancers that originate in supportive or connective tissues such as bones, tendons, cartilage, muscle and fat. Exemplary sarcomas include osteosarcoma, chondrosarcoma, leiomyosarcoma, rhabdomyosarcoma, mesothelial sarcoma, fibrosarcoma, angiosarcoma, liposarcoma, glioma or astrocytoma, myxosarcoma and mesenchymous or mixed mesodermal tumors.

Tumors can arise from most organs and tissue in the body, including, but not limited to, brain and nervous tissue, breast, cervix, ovary, uterus, colorectal, stomach and gastric tissue, kidney, liver, lung oesophagus, pancreas, prostate, skin, bone, head and neck, and lung. Exemplary brain and nervous system cancers include neurogliomas and glioblastomas. Exemplary breast cancers include human breast carcinomas, breast adenocarcinomas and invasive ductal carcinomas. Exemplary cervical cancers include epidermoid carcinomas, cervical carcinomas and HPV positive cervical cancers. Exemplary ovarian cancers include ovarian carcinomas. Exemplary colorectal cancers include colorectal carcinomas and colon colorectal adenocarcinomas. Exemplary stomach and gastric cancers include gastric adenocarcinomas, stomach adenocarcinomas and gastric carcinomas. Exemplary kidney cancers include renal cell adenocarcinomas and kidney clear cell carcinomas. Exemplary liver cancers include hepatocellular carcinomas and hepatomas. Exemplary lung cancers include small cell lung cancers, non-small cell lung cancers, lung carcinomas, lung adenocarcinomas, squamous cell carcinomas and large cell carcinomas. Exemplary esophageal cancers include esophageal squamous cell carcinoma. Exemplary pancreatic cancers include pancreatic carcinoma and pancreatic ductal adenocarcinoma. Exemplary prostate cancers include prostate carcinomas, prostate adenocarcinomas and castrate resistant prostate cancers. Exemplary skin cancers include melanomas, squamous cell carcinomas and basal cell carcinomas. Exemplary head and neck cancers include squamous cell carcinomas.

As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments a human subject is an adult, adolescent, or pediatric subject (a child). In some embodiments, a subject is suffering from a disease, disorder or condition, e.g., a disease, disorder or condition that can be treated as provided herein, e.g., a cancer or a tumor listed herein. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

A cancer that is to be treated can be staged according to the American Joint Committee on Cancer (AJCC) TNM classification system, where the tumor (T) has been assigned a stage of TX, T1, TImic, TIa, Tib, Tic, T2, T3, T4, T4a, T4b, T4c, or T4d; and where the regional lymph nodes (N) have been assigned a stage of NX, NO, N1, N2, N2a, N2b, N3, N3a, N3b, or N3c; and where distant metastasis (M) can be assigned a stage of MX, M0, or M1. A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pNO, PNO (I−), PNO (I+), PNO (mol−), PNO (mol+), PN1, PN1(mi), PNla, PNib, PNic, pN2, pN2a, pN2b, pN3, pN3a, pN3b, or pN3c.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

As used herein, “monotherapy” refers to the administration of a single active or therapeutic compound to a subject in need thereof. Preferably, monotherapy will involve administration of a therapeutically effective amount of an active compound. For example, cancer monotherapy with one of the compound of the present invention, or a pharmaceutically acceptable salt, polymorph, solvate, analog or derivative thereof, to a subject in need of treatment of cancer. Monotherapy may be contrasted with combination therapy, in which a combination of multiple active compounds is administered, preferably with each component of the combination present in a therapeutically effective amount. In one aspect, monotherapy with a compound of the present invention, or a pharmaceutically acceptable salt, polymorph or solvate thereof, is more effective than combination therapy in inducing a desired biological effect.

As used herein, “treating” or “treat” describes the management and care of a patient for the purpose of combating a disease, condition, or disorder and includes the administration of a compound of the present invention, or a pharmaceutically acceptable salt, polymorph or solvate thereof, to alleviate one or more symptoms or complications of a disease, condition or disorder, or to eliminate the disease, condition or disorder. The term “treat” can also include treatment of a cell in vitro or an animal model.

A compound of the present invention, or a pharmaceutically acceptable salt, polymorph or solvate thereof, can also be used to prevent a disease, condition or disorder, or used to identify suitable candidates for such purposes. As used herein, “preventing” or “prevent” describes reducing or eliminating the onset of the symptoms or complications of the disease, condition or disorder.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions of the invention leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

In another aspect of the invention, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present invention, or a pharmaceutically acceptable salt, polymorph, solvate, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present invention, or a pharmaceutically acceptable salt, polymorph, solvate, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating cancer can result in a reduction in the rate of cellular proliferation.

Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating cancer can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of non dividing cells in a tissue sample. The proportion of proliferating cells can be equivalent to the mitotic index.

Treating cancer can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating cancer can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleomorphism.

Treating cancer can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci USA. 100(5): 2674-8, 2003. In an aspect, cell death occurs by apoptosis.

As used herein, the term “selectively” means tending to occur at a higher frequency in one population than in another population. The compared populations can be cell populations. Preferably, a compound of the present invention, or a pharmaceutically acceptable salt, polymorph or solvate thereof, acts selectively on a cancer or precancerous cell but not on a normal cell. Preferably, a compound of the present invention, or a pharmaceutically acceptable salt, polymorph or solvate thereof, acts selectively to modulate one molecular target (e.g., p300) but does not significantly modulate another molecular target (e.g., a non-target protein). The invention also provides a method for selectively inhibiting the activity of a protein such as p300. Preferably, an event occurs selectively in population A relative to population B if it occurs greater than two times more frequently in population A as compared to population B. An event occurs selectively if it occurs greater than five times more frequently in population A. An event occurs selectively if it occurs greater than ten times more frequently in population A; more preferably, greater than fifty times; even more preferably, greater than 100 times; and most preferably, greater than 1000 times more frequently in population A as compared to population B. For example, cell death would be said to occur selectively in cancer cells if it occurred greater than twice as frequently in cancer cells as compared to normal cells.

A compound of the present invention, or a pharmaceutically acceptable salt, polymorph or solvate thereof, can modulate the activity of a molecular target (e.g., p300). Modulating refers to stimulating or inhibiting an activity of a molecular target. Preferably, a compound of the present invention, or a pharmaceutically acceptable salt, polymorph or solvate thereof, modulates the activity of a molecular target if it stimulates or inhibits the activity of the molecular target by at least 2-fold relative to the activity of the molecular target under the same conditions but lacking only the presence of said compound. More preferably, a compound of the present invention, or a pharmaceutically acceptable salt, polymorph or solvate thereof, modulates the activity of a molecular target if it stimulates or inhibits the activity of the molecular target by at least 5-fold, at least 10-fold, at least 20-fold, at least 50-fold, at least 100-fold relative to the activity of the molecular target under the same conditions but lacking only the presence of said compound. The activity of a molecular target may be measured by any reproducible means. The activity of a molecular target may be measured in vitro or in vivo. For example, the activity of a molecular target may be measured in vitro by an enzymatic activity assay or a DNA binding assay, or the activity of a molecular target may be measured in vivo by assaying for expression of a reporter gene.

A compound of the present invention, or a pharmaceutically acceptable salt, polymorph or solvate thereof, does not significantly modulate the activity of a molecular target if the addition of the compound does not stimulate or inhibit the activity of the molecular target by greater than 10% relative to the activity of the molecular target under the same conditions but lacking only the presence of said compound.

Preferably, a compound of the present invention, or a pharmaceutically acceptable salt, polymorph or solvate thereof, demonstrates this differential across the range of inhibition, and the differential is exemplified at the IC₅₀, i.e., a 50% inhibition, for a molecular target of interest.

Administering a compound of the present invention, or a pharmaceutically acceptable salt, polymorph or solvate thereof, to a cell or a subject in need thereof can result in modulation (i.e., stimulation or inhibition) of an activity of a protein of interest.

Administering a compound of the present invention, or a pharmaceutically acceptable salt, polymorph or solvate thereof, to a cell or a subject in need thereof results in modulation (i.e., stimulation or inhibition) of an activity of an intracellular target (e.g., substrate).

Preferably, an effective amount of a compound of the present invention, or a pharmaceutically acceptable salt, polymorph or solvate thereof, is not significantly cytotoxic to normal cells. A therapeutically effective amount of a compound is not significantly cytotoxic to normal cells if administration of the compound in a therapeutically effective amount does not induce cell death in greater than 10% of normal cells. A therapeutically effective amount of a compound does not significantly affect the viability of normal cells if administration of the compound in a therapeutically effective amount does not induce cell death in greater than 10% of normal cells. In an aspect, cell death occurs by apoptosis.

Contacting a cell with a compound of the present invention, or a pharmaceutically acceptable salt, polymorph or solvate thereof, can induce or activate cell death selectively in cancer cells. Administering to a subject in need thereof a compound of the present invention, or a pharmaceutically acceptable salt, polymorph or solvate thereof, can induce or activate cell death selectively in cancer cells. Contacting a cell with a compound of the present invention, or a pharmaceutically acceptable salt, polymorph or solvate thereof, can induce cell death selectively in one or more cells affected by a cell proliferative disorder. Preferably, administering to a subject in need thereof a compound of the present invention, or a pharmaceutically acceptable salt, polymorph or solvate thereof, induces cell death selectively in one or more cells affected by a cell proliferative disorder.

One skilled in the art may refer to general reference texts for detailed descriptions of known techniques discussed herein or equivalent techniques. These texts include Ausubel et al, Current Protocols in Molecular Biology, John Wiley and Sons, Inc. (2005); Sambrook et al, Molecular Cloning, A Laboratory Manual (3rd edition), Cold Spring Harbor Press, Cold Spring Harbor, New York (2000); Coligan et al, Current Protocols in Immunology, John Wiley & Sons, N.Y.; Enna et al, Current Protocols in Pharmacology, John Wiley & Sons, N.Y.; Fingl et al, The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the invention.

### Dosing Regimen

An exemplary treatment regime entails administration once daily, twice daily, three times daily, every second day, twice per week, once per week. The composition of the invention is usually administered on multiple occasions. Intervals between single dosages can be, for example, less than a day, daily, every second day, twice per week, or weekly. The composition of the invention may be given as a continuous uninterrupted treatment. In an exemplary treatment regimen the compound of formula (I) or formula (Ia) according to the invention can be administered from 0.1-1500 mg per day.

As used herein, the term “therapeutically effective amount” refers to an amount that produces a desired effect (e.g., a desired biological, clinical, or pharmacological effect) in a subject or population to which it is administered. In some embodiments, the term refers to an amount statistically likely to achieve the desired effect when administered to a subject in accordance with a particular dosing regimen (e.g., a therapeutic dosing regimen). In some embodiments, the term refers to an amount sufficient to produce the effect in at least a significant percentage (e.g., at least about 25%, about 30%, about 40%, about 50%, about 60%, about 70%, about 80%, about 90%, about 95%, or more) of a population that is suffering from and/or susceptible to a disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be an amount that provides a particular desired response in a significant number of subjects when administered to patients in need of such treatment, e.g., in at least about 25%, about 30%, about 40%, about 50%, about 60%, about 70%, about 80%, about 90%, about 95%, or more patients within a treated patient population. In some embodiments, reference to a therapeutically effective amount may be a reference to an amount sufficient to induce a desired effect as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

In some embodiments, a compound or pharmaceutical composition for use in accordance with the present disclosure is formulated, dosed, and/or administered in a therapeutically effective amount using pharmaceutical compositions and dosing regimens that are consistent with good medical practice and appropriate for the relevant agent(s) and subject(s). In principle, compounds and pharmaceutical compositions can be administered by any appropriate method known in the art, including, without limitation, oral, mucosal, by-inhalation, topical, buccal, nasal, rectal, or parenteral (e.g. intravenous, infusion, intratumoral, intranodal, subcutaneous, intraperitoneal, intramuscular, intradermal, transdermal, or other kinds of administration involving physical breaching of a tissue of a subject and administration of the therapeutic composition through the breach in the tissue). In some embodiments, the compound or pharmaceutical composition is administered directly to the tumor (e.g., by intratumoral injection).

In some embodiments, a dosing regimen for a particular active agent may involve intermittent or continuous (e.g., by perfusion or other slow release system) administration, for example to achieve a particular desired pharmacokinetic profile or other pattern of exposure in one or more tissues or fluids of interest in the subject receiving therapy.

In some embodiments, different agents administered in combination may be administered via different routes of delivery and/or according to different schedules. Alternatively or additionally, in some embodiments, one or more doses of a first active agent is administered substantially simultaneously with, and in some embodiments via a common route and/or as part of a single composition with, one or more other active agents.

Factors to be considered when optimizing routes and/or dosing schedule for a given therapeutic regimen may include, for example, the particular indication being treated, the clinical condition of a subject (e.g., age, overall health, prior therapy received and/or response thereto) the site of delivery of the agent, the nature of the agent (e.g. an antibody or other polypeptide-based compound), the mode and/or route of administration of the agent, the presence or absence of combination therapy, and other factors known to medical practitioners. For example, in the treatment of cancer, relevant features of the indication being treated may include, for example, one or more of cancer type, stage, location.

In some embodiments, one or more features of a particular pharmaceutical composition and/or of a utilized dosing regimen may be modified over time (e.g., increasing or decreasing the amount of active agent in any individual dose, increasing or decreasing time intervals between doses), for example in order to optimize a desired therapeutic effect or response (e.g., inhibition or modulation of a p300 gene or gene product).

In general, type, amount, and frequency of dosing of compounds or pharmaceutical compositions in accordance with the present invention are governed by safety and efficacy requirements that apply when one or more relevant agent(s) is/are administered to a mammal, preferably a human. In general, such features of dosing are selected to provide a particular, and typically detectable, therapeutic response as compared to what is observed absent therapy.

In some embodiments, a “therapeutically effective amount” or “therapeutically effective dose” is an amount of a compound or pharmaceutical composition of the disclosure, or a combination of two or more compounds or pharmaceutical compositions of the disclosure, or a combination of a compound or pharmaceutical composition of the disclosure with one or more additional therapeutic agent(s), which inhibits, totally or partially, the progression of the condition or alleviates, at least partially, one or more symptoms of the condition. In some embodiments, a therapeutically effective amount can be an amount which is prophylactically effective. In some embodiments, an amount which is therapeutically effective may depend upon a patient's size and/or gender, the condition to be treated, severity of the condition and/or the result sought. In some embodiments, a therapeutically effective amount refers to that amount that results in amelioration of at least one symptom in a patient. In some embodiments, for a given patient, a therapeutically effective amount may be determined by methods known to those of skill in the art.

In some embodiments, toxicity and/or therapeutic efficacy of a compound or pharmaceutical composition of the disclosure can be determined by standard pharmaceutical procedures in cell cultures or experimental animals, e.g., for determining the maximum tolerated dose (MTD) and the ED₅₀ (effective dose for 50% maximal response). Typically, the dose ratio between toxic and therapeutic effects is the therapeutic index; in some embodiments, this ratio can be expressed as the ratio between MTD and ED₅₀. Data obtained from such cell culture assays and animal studies can be used in formulating a range of dosage for use in humans.

In some embodiments, dosage may be guided by monitoring the effect of a compound or pharmaceutical composition of the disclosure on one or more pharmacodynamic markers of p300 function in diseased or surrogate tissue. For example, cell culture or animal experiments can be used to determine the relationship between doses required for changes in pharmacodynamic markers such as p300 downstream target genes or p53 acetylation and doses required for therapeutic efficacy can be determined in cell culture or animal experiments or early stage clinical trials. In some embodiments, dosage of a compound or pharmaceutical composition of the disclosure lies preferably within a range of circulating concentrations that include the ED₅₀ with little or no toxicity. In some embodiments, dosage may vary within such a range, for example depending upon the dosage form employed and/or the route of administration utilized. The exact formulation, route of administration and dosage can be chosen by the individual physician in view of the patient's condition. In the treatment of crises or severe conditions, administration of a dosage approaching the MTD may be required to obtain a rapid response.

In some embodiments, dosage amount and/or interval may be adjusted individually, for example to provide plasma levels of an active moiety which are sufficient to maintain, for example a desired effect, or a minimal effective concentration (MEC) for a period of time required to achieve therapeutic efficacy. In some embodiments, MEC for a particular compound or pharmaceutical composition of the disclosure can be estimated, for example, from in vitro data and/or animal experiments. Dosages necessary to achieve the MEC will depend on individual characteristics and route of administration. In some embodiments, high pressure liquid chromatography (HPLC) assays or bioassays can be used to determine plasma concentrations.

In some embodiments, dosage intervals can be determined using the MEC value. In certain embodiments, a compound or pharmaceutical composition of the disclosure should be administered using a regimen which maintains plasma levels above the MEC for 10-90% of the time, preferably between 30-90% and most preferably between 50-90% until the desired amelioration of a symptom is achieved. In other embodiments, different MEC plasma levels will be maintained for differing amounts of time. In cases of local administration or selective uptake, the effective local concentration of the drug may not be related to plasma concentration.

One of skill in the art can select from a variety of administration regimens and will understand that an effective amount of a particular a compound or pharmaceutical composition of the disclosure may be dependent on the subject being treated, on the subject's weight, the severity of the affliction, the manner of administration and/or the judgment of the prescribing physician.

### Combination Therapy

In some embodiments, compounds or pharmaceutical compositions of the disclosure can be administered to a subject in need thereof as a cancer monotherapy. Alternatively, or in addition, compounds or pharmaceutical compositions of the disclosure can be administered to a subject in need thereof in combination with at least one additional cancer therapy.

In some embodiments, the at least one additional cancer therapy comprises a standard of care for the cancer of the subject. As used herein, “standard of care” refers to a treatment of a particular cancer that is accepted by persons of skill in the art as the generally accepted treatment for that indication, and whose practice is common amongst medical professionals. For example, standard of care for primary tumors that can be surgically resected without undue risk to the subject comprises surgical removal of the tumor. The person of ordinary skill in the art will readily understand what is a “standard of care” for a particular cancer indication.

In some embodiments, the at least one additional cancer therapy comprises surgical resection of the cancer, radiation therapy, or a combination thereof.

In some embodiments, compounds or pharmaceutical compositions of the disclosure can be used in combination with another therapeutic agent to treat cancer in the subject in a combinational therapy. In some embodiments, the combinational therapy is in addition to a standard of care therapies, surgical resection and/or radiation therapy.

In some embodiments, compounds or pharmaceutical compositions of the disclosure can optionally contain, and/or be administered in combination with, one or more additional therapeutic agents, such as a cancer therapeutic agent, e.g., a chemotherapeutic agent or a biological agent.

An additional agent can be, for example, a therapeutic agent that is e.g., an anti-cancer agent, or an agent that ameliorates a symptom associated with the disease or condition being treated. The additional agent also can be an agent that imparts a beneficial attribute to the therapeutic composition (e.g., an agent that affects the viscosity of the composition). For example, in some embodiments, compounds or pharmaceutical compositions of the disclosure are administered to a subject who has received, is receiving, and/or will receive therapy with another therapeutic agent or modality (e.g., with a chemotherapeutic agent, surgery, radiation, or a combination thereof).

Some embodiments of combination therapy modalities provided by the present disclosure provide, for example, administration of compounds or pharmaceutical compositions of the disclosure and additional cancer therapeutic agent(s) in a single pharmaceutical formulation.

Some embodiments provide administration of compounds or pharmaceutical compositions of the disclosure and administration of additional cancer therapeutic agent(s) in separate pharmaceutical formulations. In some embodiments, the compounds or pharmaceutical compositions of the disclosure and the additional cancer therapeutic agent are administered simultaneously. Simultaneous administration can be by the same modality (e.g., both by oral administration), or by different modalities (e.g., one oral, one injected). In some embodiments, the compounds or pharmaceutical compositions of the disclosure and the additional cancer therapeutic agent are administered in temporal proximity. For example, the compounds or pharmaceutical compositions of the disclosure and the additional cancer therapeutic agent are administered within 1 minute, 2 minutes, 5 minutes, 10 minutes, 15 minutes, 30 minutes, 1 hour, 2 hours, 3 hours, 4 hours, 5 hours, 6 hours, 12 hours or 24 hours of each other. In some embodiments, the compounds or pharmaceutical compositions of the disclosure and the additional cancer therapeutic agent are administered in sequence. For example, the compounds or pharmaceutical compositions of the disclosure and the additional cancer therapeutic agent can be administered in an alternating sequence.

In some embodiments, the at least one additional cancer therapeutic agent comprises a chemotherapeutic agent.

Examples of chemotherapeutic agents that can be used in combination with compound or pharmaceutical composition described herein include platinum compounds (e.g., cisplatin, carboplatin, and oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, and bendamustine), antitumor antibiotics (e.g., daunorubicin, doxorubicin, idarubicin, epirubicin, mitoxantrone, bleomycin, mytomycin C, plicamycin, and dactinomycin), taxanes (e.g., paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, and nelarabine), topoisomerase inhibitors (e.g., topotecan and irinotecan), hypomethylating agents (e.g., azacitidine and decitabine), proteasome inhibitors (e.g., bortezomib), epipodophyllotoxins (e.g., etoposide and teniposide), DNA synthesis inhibitors (e.g., hydroxyurea), vinca alkaloids (e.g., vincristine, vindesine, vinorelbine, and vinblastine), tyrosine kinase inhibitors (e.g., imatinib, dasatinib, nilotinib, sorafenib, and sunitinib), nitrosoureas (e.g., carmustine, fotemustine, and lomustine), hexamethylmelamine, mitotane, angiogenesis inhibitors (e.g., thalidomide and lenalidomide), steroids (e.g., prednisone, dexamethasone, and prednisolone), hormonal agents (e.g., enzalutamide, tamoxifen, raloxifene, leuprolide, bicalutamide, granisetron, and flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, and oxaprozin), selective cyclooxygenase-2 (COX-2) inhibitors, or any combination thereof.

In some embodiments, the additional agent affects (e.g., inhibits) histone modifications, such as histone acetylation or histone methylation. In certain embodiments, an additional anticancer agent is selected from the group consisting of chemotherapeutics (such as 2CdA, 5-FU, 6-Mercaptopurine, 6-TG, Abraxane™, Accutane®, Actinomycin-D, Adriamycin®, Alimta®, all-trans retinoic acid, amethopterin, Ara-C, Azacitadine, BCNU, Blenoxane®, Camptosar®, CeeNU®, Clofarabine, Clolar™, Cytoxan®, daunorubicin hydrochloride, DaunoXome®, Dacogen®, DIC, Doxil®, Ellence®, Eloxatin®, Emcyt®, etoposide phosphate, Fludara®, FUDR®, Gemzar®, Gleevec®, hexamethylmelamine, Hycamtin®, Hydrea®, Idamycin®, Ifex®, ixabepilone, Ixempra®, L-asparaginase, Leukeran®, liposomal Ara-C, LPAM, Lysodren, Matulane®, mithracin, Mitomycin-C, Myleran®, Navelbine®, Neutrexin®, nilotinib, Nipent®, Nitrogen Mustard, Novantrone®, Oncaspar®, Panretin®, Paraplatin®, Platinol®, prolifeprospan 20 with carmustine implant, Sandostatin®, Targretin®, Tasigna®, Taxotere®, Temodar®, TESPA, Trisenox®, Valstar®, Velban®, Vidaza™, vincristine sulfate, VM 26, Xeloda® and Zanosar®); biologies (such as Alpha Interferon, Bacillus Calmette-Guerin, Bexxar®, Campath®, Ergamisol®, Erlotinib, Herceptin®, Interleukin-2, Iressa®, lenalidomide, Mylotarg®, Ontak®, Pegasys®, Revlimid®, Rituxan®, Tarceva™, Thalomid®, Velcade® and Zevalin™); small molecules (such as Tykerb@); corticosteroids (such as dexamethasone sodium phosphate, DeltaSone® and Delta-Cortef®); hormonal therapies (such as Arimidex®, Aromasin®, Casodex®, Cytadren®, Eligard®, Eulexin®, Evista®, Faslodex®, Femara®, Halotestin®, Megace®, Nilandron®, Nolvadex®, Plenaxis™ and Zoladex®); and radiopharmaceuticals (such as Iodotope®, Metastron®, Phosphocol® and Samarium SM-153).

Examples of biological agents that can be used in combination with the compositions and methods described herein include monoclonal antibodies (e.g., rituximab, cetuximab, obinutuzumab, ofatumumab, ibritumomab, brentuximab, bevacizumab, panitumumab, pembrolizumab, tositumomab, trastuzumab, alemtuzumab, gemtuzumab ozogamicin, bevacizumab, catumaxomab, denosumab, obinutuzumab, ofatumumab, ramucirumab, pertuzumab, ipilimumab, nivolumab, nimotuzumab, lambrolizumab, pidilizumab, siltuximab, tremelimumab, or others known in the art), enzymes (e.g., L-asparaginase), cytokines (e.g., interferons and interleukins), growth factors (e.g., colony stimulating factors and erythropoietin) or inhibitors thereof, cancer vaccines, gene therapy vectors, or any combination thereof. In some embodiments, the growth factor inhibitor comprises an inhibitor of vascular endothelial growth factor A (VEGFA). In some embodiments, the inhibitor of VEGFA comprises Avastin® (bevacizumab).

In some embodiments, biological agents comprise adoptive cell therapies. For example, chimeric antigen receptor T cell (CAR-T) therapies. In some embodiments, the adoptive cell therapy is autologous. In some embodiments, the adoptive cell therapy is allogeneic.

In some embodiments, the at least one additional cancer therapeutic agent comprises an immune checkpoint inhibitor. Immune checkpoint inhibitors target immune checkpoints, which regulate the immune system, and under certain circumstances, can prevent the immune system from targeting tumors. In some embodiments, the immune checkpoint comprises a PD-1/PD-L1 immune checkpoint. In some embodiments, the immune checkpoint comprises a CLTA-4 immune checkpoint. In some embodiments, the immune checkpoint inhibitor is an antibody or a small molecule. Exemplary PD-1 inhibitors include, but are not limited, nivolumab and pembrolizumab. Exemplary PD-L1 inhibitors include, but are not limited to, atezolizumab, avelumab and durvalumab. Exemplary CLTA-4 inhibitors include, but are not limited to, ipilimumab.

The additional agents that can be used in combination with compositions and methods of the disclosure as set forth above are for illustrative purposes and not intended to be limiting. The combinations embraced by this disclosure, include, without limitation, one or more compounds or pharmaceutical compositions as provided herein and at least one additional agent selected from the categories or lists above or otherwise provided herein. The compounds and pharmaceutical compositions of the disclosure can also be used in combination with one or with more than one additional agent, e.g., with two, three, four, five, or six, or more, additional agents.

In some embodiments, treatment methods described herein are performed on subjects for which other treatments of the medical condition have failed or have had less success in treatment through other means, e.g., in subjects having a cancer refractory to standard-of-care treatment. Additionally, the treatment methods described herein can be performed in conjunction with one or more additional treatments of the medical condition, e.g., in addition to or in combination with standard-of-care treatment. For instance, the method can comprise administering a cancer regimen, e.g., nonmyeloablative chemotherapy, surgery, hormone therapy, and/or radiation, prior to, substantially simultaneously with, in temporal proximity to, in sequence with or after the administration of a compound or pharmaceutical composition described herein.

### Additional Methods

The invention encompasses methods comprising providing at least one compound, measuring the activity of the compound and determining if the activity of the compound is above or below a predetermined level.

Methods of measuring the activity of a compound will be readily apparent to one of ordinary skill in the art. Exemplary methods include measuring growth-inhibitory concentration (GI50) in vitro in a cell proliferation assay or a colony survival assay. Cell proliferation can be measured using any technique known in the art. For example, cell proliferation can be measured by measuring colony formation using stains such as Crystal Violet/DBPS and measuring 600 nm absorbance. Alternatively, or in addition, cells can be treated with a dye that permeabilizes the cells and reacts with certain enzymes to provide a measure of metabolic activity (for example, MTT or WST-1). Proliferation can be measured using fluorescence dyes such as CyQUANT (ThermoFisher Scientific). Alternatively, or in addition, cell proliferation can be measured by examining one or more proliferation markers, such as BrdU incorporation or proliferating cell nuclear antigen (PCNA) expression.

Alternatively, or in addition, the method of measuring activity of a compound of the disclosure comprises measuring an effect of the compound on tumor growth in an animal. Exemplary animal cancer models include, but are not limited to, patient derived xenograft (PDX) cancer models, transgenic models and gene knock out or gene knock in models that modify one or more tumor suppressor or oncogenes and syngeneic models. In a PDX model, cancer cells derived from a patient or cell line isolated or derived from a cancer of interest are transplanted into an immune deficient animal. In some embodiments, the immune deficient animal is a severely compromised immune deficient (SCID) mouse, a NOD-SCID mouse, or a recombination-activity gene 2 (Rag2) knockout mouse, which prevents transplant rejection. In a syngeneic model, e.g. a syngeneic mouse model, tumor tissues from the same genetic background as the given immuno-competent mouse strain are transplanted into the mouse to induce tumor formation. Optionally, cancer cells can be transformed with one or more markers to facilitate analysis, for example, a Luciferase gene to mark PDX acute myeloid leukemia cells transplanted into an immune deficient mouse via bone marrow engraftment. In some embodiments, the animal model is an animal that has been genetically modified to contain mutations that lead to cancer, for example by knocking out one or more genes which suppress cancer formation, or introducing (knocking in) one or more mutations that cause cancer, optionally in a tissue specific manner using tissue specific drivers and recombination cassettes such as Cre-LOX. For example, mice that are engineered to be p53+/− can be used to study cancers as these animals spontaneously give rise to tumors in clones of cells that have lost the wild type p53 allele.

Alternatively, or in addition, the method of measuring activity comprises measuring a change in RNA expression of certain genes in tumor-derived cell cultures, blood, diseased tissues or diseased organs of treated individuals. The gene or genes can be, for example, genes that are regulated by p300. p300 regulation of target genes can be either direct (e.g. transcriptional regulation, through p300 activity at the cognate gene promoter), or indirect (e.g., through p300 regulation of upstream transcription factors involved in regulation of a target gene). Exemplary p300 target genes include, but are not limited to, androgen response genes such as kallikrein related peptidase 3/prostate-specific antigen (KLK3/PSA), transmembrane serine protease 2 (TMPRSS2) and solute carrier family 45 member 3 (SLC45A3), VEGF and P53.

Alternatively, or in addition, the method of measuring activity comprises measuring the change in RNA expression of p300 target genes in vitro in cell culture assays. Methods of measuring RNA expression of p300 target genes will be readily apparent to one of ordinary skill in the art. For example, levels of RNA expression can be measured using high throughput sequencing methods, microarrays, reverse transcription polymerase chain reaction (RT-PCR), quantitative RT-PCR (RT-qPCR) and droplet digital PCR (ddPCR) as well as any other method known in the art. In some embodiments, the method of measuring activity comprises measuring the change in RNA expression of Androgen Receptor—responsive genes in vitro in cell culture assays (for example, KLK3, TMPRSS2 and/or SLC45A3). In some embodiments, the method of measuring activity comprises measuring the amount of Tumor-specific Protein 53 (p5³) in vitro in cell culture assays.

In some embodiments, the method of measuring activity comprises measuring the amount of acetylated p53 lysine 382 (p53K382Ac) in vitro in cell culture assays. The amount of acetylated p53 lysine 382 can be measured, for example, by using a p53K382Ac specific antibody and Western Blot.

In some embodiments, the method of measuring activity comprises measuring the amount of Prostate-Specific Antigen protein in serum of treated individuals. The amount of PSA can be measured, for example, with a PSA specific antibody and by Western Blot or ELISA.

In some embodiments, the method of measuring activity comprises measuring the amount of Vascular Endothelial Growth Factor (VEGF) protein in serum, diseased tissues or diseased organs of treated individuals. The amount of VEGF protein can be measured, for example, with a VEGF specific antibody and by Western Blot or ELISA. Alternatively, or in addition, the method of measuring activity comprises measuring the amount of VEGF RNA in serum, diseased tissues or diseased organs of treated individuals.

### Kits and Article of Manufacture

The disclosure provides kits comprising the compounds and pharmaceutical compositions of the disclosure and instructions for use in treating a cancer in a subject in need thereof.

In some embodiments, the kits further comprise at least one additional cancer therapeutic agent. Any additional cancer therapeutic described herein is envisaged as being within the scope of a kit of the disclosure. In some embodiments, the compounds or pharmaceutical compositions of the disclosure and the at least one additional cancer therapeutic are different compositions. In some embodiments, the compounds or pharmaceutical compositions of the disclosure and the at least one additional cancer therapeutic formulated in the same composition.

In some embodiments, the at least one additional cancer therapeutic agent comprises a chemotherapeutic agent. Exemplary chemotherapeutic agents include, but are not limited to a platinum compound, an alkylating agent, an antitumor antibiotic, a taxane, an antimetabolite, a nucleoside analog, a topoisomerase inhibitor, a hypomethylating agent, a proteasome inhibitor, an epipodophyllotoxin, a DNA synthesis inhibitor, a vinca alkaloid, a tyrosine kinase inhibitor, a nitrosourea, hexamethylmelamine, mitotane, an angiogenesis inhibitor, a steroid, a hormonal agent, an aromatase inhibitor, arsenic trioxide, tretinoin, a nonselective cyclooxygenase inhibitor, a selective cyclooxygenase-2 (COX-2) inhibitors, or a combination thereof.

In some embodiments, the additional cancer therapeutic agent comprises a biological agent. Exemplary biological agents include, but are not limited to, an antibody therapy, an adoptive cell therapy, an enzyme, a cytokine, a growth factor or inhibitor thereof, a gene therapy a cancer vaccine or a combination thereof.

In some embodiments, the additional cancer therapeutic agent comprises an immune checkpoint inhibitor. The immune checkpoint inhibitor can be a small molecule, or an antibody. Exemplary antibodies include, but are not limited to, nivolumab, pembrolizumab, atezolizumab, avelumab, durvalumab or ipilimumab.

Kits comprising the compounds and pharmaceutical compositions of the disclosure are for the use in treating a cancer in a subject. Exemplary cancers include liquid tumors such as leukemias and lymphomas, and solid tumors such as carcinomas, sarcomas, myelomas, germ cell tumors, carcinoid tumors, neuroendocrine tumors or tumors of mixed type. Exemplary cancers include, but are not limited to prostate cancer, colon cancer, head-and-neck cancer, cervical cancer, brain or nervous system cancer, ovarian cancer, stomach or gastric cancer, kidney cancer, liver cancer, oesophageal cancer, pancreatic cancer, skin cancer and lung cancer.

Articles of manufacture include, but are not limited to labels, instructional pamphlets, vials and syringes.

### ENUMERATED EMBODIMENTS

The invention may be defined by reference to the following enumerated, illustrative embodiments:

1. A compound of formula (Ia)

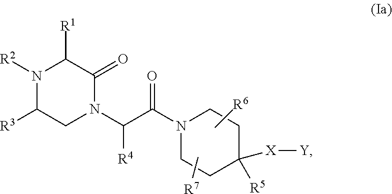

or a pharmaceutically acceptable salt, hydrate, solvate or stereoisomer thereof, wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by cycloalkyl, aryl or heteroaryl, wherein the cycloalkyl, aryl or the heteroaryl is optionally substituted by halogen, C₁₋₄ alkyl or C₃₋₅ cycloalkyl;
R² is selected from H, C(O)R¹⁴, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by cycloalkyl, aryl or heteroaryl, wherein the cycloalkyl, aryl or the heteroaryl is optionally substituted by halogen, C₁₋₄ alkyl or C₃₋₅ cycloalkyl; with the proviso that when R² is C(O)NR¹⁵R¹⁵, both R¹⁵ can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, or C₄₋₇ cycloalkenyl, all optionally substituted by halogen, OR⁸, NR⁸R¹¹, or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by cycloalkyl, aryl or heteroaryl, wherein the cycloalkyl, aryl or the heteroaryl is optionally substituted by halogen, C₁₋₄ alkyl or C₃₋₅ cycloalkyl;
R⁵ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸; and wherein R⁵ can form a ring with any part of X or Y, wherein the ring optionally contains a carbonyl group;
R⁶ is selected from H, C₁₋₇ alkyl, C₂₋₇ alkenyl, C₂₋₇ alkynyl, C₃₋₇ cycloalkyl, C₄₋₇ cycloakenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; and wherein R⁶ can form a ring with any part of X; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl;
X is selected from a bond, C₁₋₇ alkanediyl, C₂₋₇ alkenediyl, C₂₋₇ alkynediyl, C₃₋₉ cycloalkanediyl, C₄₋₆ cycloalkenediyl, —O—, C₁₋₃ alkanediyl-O—, —O—C₁₋₇ alkanediyl, —O—C₃₋₉ cycloalkanediyl, C₁₋₃ alkanediyl-O—C₁₋₇ alkanediyl, C₁₋₇ heteroalkanediyl, or —S—C₁₋₇ alkanediyl; and wherein X can form a ring or a polycyclic system with any part of R⁵, R⁶, or Y, wherein the ring optionally contains a carbonyl group;
Y is selected from H, C(O)NR¹⁰R¹², C(O)OR¹⁰, R¹⁰NC(O)NR¹⁰R¹², OC(O)R¹⁰, OC(O)NR¹⁰R¹², S(O)ₙR⁸ wherein n is 0, 1 or 2, SO₂NR¹⁰R¹², NR¹⁰SO₂R¹⁰, NR¹⁰R¹², HNCOR⁸, CN, C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or aryl, heteroaryl wherein the aryl or heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R⁹ is selected from H, halogen, C₁₋₅ alkyl, C₂₋₅ alkenyl, C₂₋₅ alkynyl, C₃₋₅ cycloalkyl, C₁₋₅ alkyl-OR⁸, C₁₋₅ alkyl-SR⁸, C₁₋₅ alkyl-NR⁸R¹¹, C₁₋₅ alkyl-C(O)OR⁸, C₁₋₅ alkyl-C(O)NR⁸R¹¹, C₁₋₅ alkyl-C(O)R¹⁰, CN, C(O)R⁸, C(O)NR⁸R¹¹, C(O)OR⁸, NR⁸C(O)NR⁸R¹¹, OC(O)NR⁸R¹¹, SO₂NR⁸R¹¹, NR⁸SO₂R⁸, OR⁸, NR⁸R¹¹, or S(O)ₙR⁸ wherein n is 0, 1 or 2;
R¹⁰ and R¹² are each independently selected from H, C₁₋₇ alkyl, C₂₋₇ alkenyl, C₂₋₇ alkynyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen, OR⁸, or NR⁸R¹¹;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl; and
each R¹⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.
   2. A compound of formula (I)

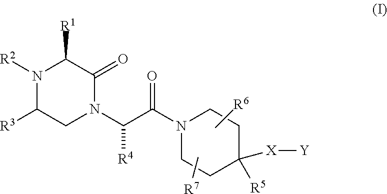

or a pharmaceutically acceptable salt, hydrate, solvate or stereoisomer thereof, wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R² is selected from H, C(O)R¹⁴, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR⁸, C₁₋₃ alkanediyl-O—C₁₋₃ alkanediyl-O—C₁₋₃ alkanediyl, C₁₋₅ alkyl-NHCOR¹³, or C₁₋₃ alkyl substituted by aryl or heteroaryl, wherein the aryl or the heteroaryl is optionally substituted by halogen, C₁₋₄ alkyl or C₃₋₅ cycloalkyl; with the proviso that when R² is C(O)NR¹⁵R¹⁵, both R¹⁵ can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
R⁵ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR⁸, or SR⁸; and wherein R⁵ can form a ring with any part of X or Y, wherein the ring optionally contains a carbonyl group;
R⁶ is selected from H, C₁₋₇ alkyl, C₂₋₇ alkenyl, C₂₋₇ alkynyl, C₃₋₇ cycloalkyl, C₄₋₇ cycloalkenyl, all optionally substituted by halogen, OR⁸, NR⁸R¹¹; or C₁₋₃ alkyl substituted by C(O)NR⁸R¹¹; C₁₋₃ alkyl substituted by aryl or heteroaryl, wherein the aryl or the heteroaryl is optionally substituted by halogen, C₁₋₄ alkyl or C₃₋₅ cycloalkyl; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl;
X is selected from a bond, C₁₋₇ alkanediyl, C₂₋₇ alkenediyl, C₂₋₇ alkynediyl, C₃₋₆ cycloalkanediyl, C₄₋₆ cycloalkenediyl, —O—, C₁₋₃ alkanediyl-O—, —O—C₁₋₇ alkanediyl, C₁₋₃ alkanediyl-O—C₁₋₇ alkanediyl, C₁₋₇ heteroalkanediyl, or —S—C₁₋₇ alkanediyl; and wherein X can form a ring with any part of R⁵ or Y, wherein the ring optionally contains a carbonyl group;
Y is selected from H, C(O)NR¹⁰R¹², C(O)OR¹⁰, R¹⁰NC(O)NR¹⁰R¹², OC(O)R¹⁰, OC(O)NR¹⁰R¹², S(O)ₙR⁸ wherein n is 0, 1 or 2, SO₂NR¹⁰R¹², NR¹⁰SO₂R¹⁰, NR¹⁰R¹², HNCOR⁸, CN, C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; aryl, or heteroaryl wherein the aryl or heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R⁹ is selected from H, halogen, C₁₋₅ alkyl, C₂₋₅ alkenyl, C₂₋₅ alkynyl, C₃₋₅ cycloalkyl, C₁₋₅ alkyl-OR⁸, C₁₋₅ alkyl-SR^{B}, C₁₋₅ alkyl-NR⁸R¹¹, C₁₋₅ alkyl-C(O)OR⁸, C₁₋₅ alkyl-C(O)NR⁸R¹¹, C₁₋₅ alkyl-C(O)R¹⁰, CN, C(O)R⁸, C(O)NR⁸R¹¹, C(O)OR⁸, NR⁸C(O)NR⁸R¹¹, OC(O)NR⁸R¹¹, SO₂NR⁸R¹¹, NR⁸SO₂R⁸, OR⁸, NR⁸R¹¹, or S(O)ₙR⁸ wherein n is 0, 1 or 2;
R¹⁰ and R¹² are each independently selected from H, C₁₋₇ alkyl, C₂₋₇ alkenyl, C₂₋₇ alkynyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen, OR⁸, or NR⁸R¹¹;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl; and
each R¹⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR⁸.
   3. The compound according to any one of the previous embodiments, wherein R¹ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl.
   4. The compound according to any one of the previous embodiments, wherein R¹ is selected from C₂₋₇ alkyl, C₃₋₇ cycloalkyl, or C₁₋₃ alkyl substituted by aryl or heteroaryl, wherein the aryl or the heteroaryl is optionally substituted by halogen, C₁₋₄ alkyl or C₃₋₅ cycloalkyl.
   5. The compound according to any one of the previous embodiments, wherein R¹ is selected from C₃₋₇ alkyl, C₃₋₇ cycloalkyl, or C₁₋₃ alkyl substituted by aryl or heteroaryl.
   6. The compound according to any one of the previous embodiments, wherein R² is selected from H, C(O)R¹⁴, C(O)OR¹⁵, C₁₋₇ alkyl, C₃₋₇ cycloalkyl, C₁₋₃ alkanediyl-O—C₁₋₃ alkanediyl-O—C₁₋₃ alkanediyl, C₁₋₅ alkyl-OR⁸, C₁₋₅ alkyl-NHCOR¹³, or C₁₋₃ alkyl substituted by aryl, wherein the aryl is optionally substituted by halogen, C₁₋₄ alkyl or C₃₋₅ cycloalkyl.
   7. The compound according to any one of the previous embodiments, wherein R² is selected from H, C(O)R¹⁴, wherein R¹⁴ is C₁₋₇ alkyl; C₁₋₇ alkyl, C₃₋₇ cycloalkyl, C₁₋₅ alkyl-OR⁸, C₁₋₅ alkyl-NHCOR¹³, wherein R¹³ is pentylamino-5-oxopentyl-7-thia-2,4-diazabicyclo[3.3.0]octan-3-one; or C₁₋₃ alkyl substituted by aryl, wherein the aryl is optionally substituted by halogen, C₁₋₄ alkyl or C₃₋₅ cycloalkyl.
   8. The compound according to any one of the previous embodiments, wherein R³ and R⁷ are each independently selected from H, C₁₋₇ alkyl, C₂₋₇ alkenyl, C₂₋₇ alkynyl, C₃₋₇ cycloalkyl, or C₄₋₇ cycloalkenyl.
   9. The compound according to any one of the previous embodiments, wherein R³ and R⁷ are H.
   10. The compound according to any one of the previous embodiments, wherein R⁴ is selected from C₁₋₇ alkyl, C₃₋₇ cycloalkyl, or C₁₋₃ alkyl substituted by aryl or heteroaryl, wherein the aryl or the heteroaryl is optionally substituted by halogen, C₁₋₄ alkyl or C₃₋₅ cycloalkyl.
   11. The compound according to any one of the previous embodiments, wherein R⁴ is selected from C₂₋₇ alkyl, C₃₋₇ cycloalkyl, or C₁₋₃ alkyl substituted by aryl or heteroaryl, wherein the aryl or the heteroaryl is optionally substituted by halogen, C₁₋₄ alkyl or C₃₋₅ cycloalkyl.
   12. The compound according to any one of the previous embodiments, wherein R⁴ is selected from C₃₋₇ alkyl, C₃₋₇ cycloalkyl, or C₁₋₃ alkyl substituted by aryl or heteroaryl.
   13. The compound according to any one of the previous embodiments, wherein the compound is of any one of Formulae (IIa), (IIb), or (IIc):

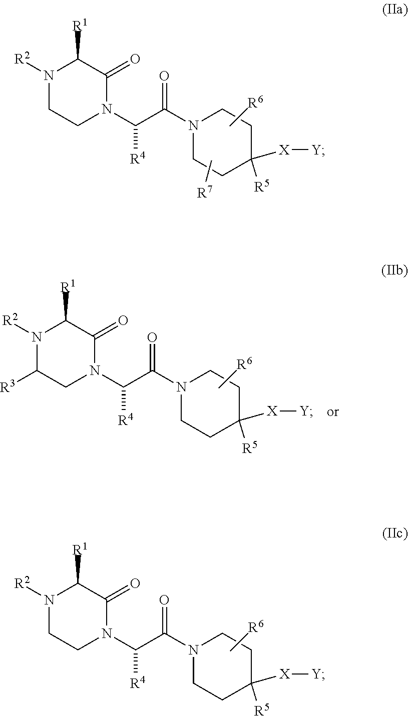

or a pharmaceutically acceptable salt, hydrate, solvate or stereoisomer thereof, wherein R¹, R², R³, R⁴, R⁵, R⁶, R⁷, X, and Y are as described herein.
14. The compound according to any one of the previous embodiments, wherein the compound is of any one of Formulae (IIIa), (IIIb), (IIIc), or (IIId):

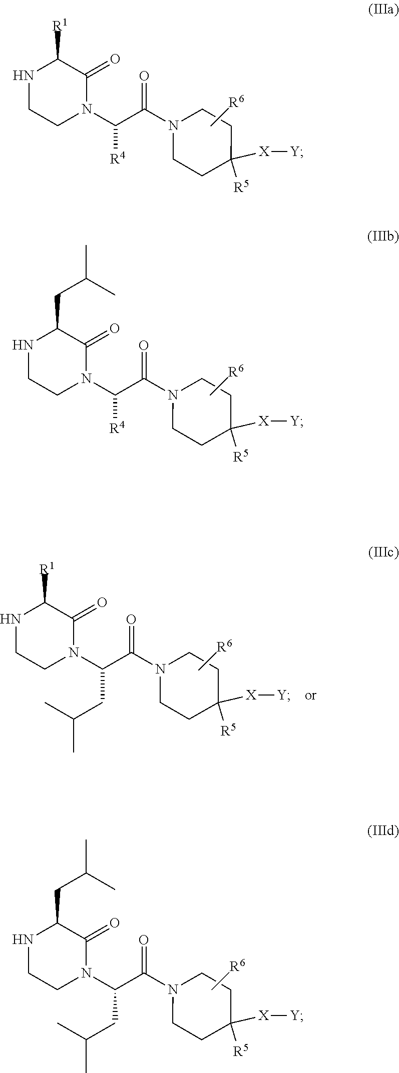

or a pharmaceutically acceptable salt, hydrate, solvate or stereoisomer thereof, wherein R¹, R⁴, R⁵, R⁶, X, and Y are as described herein.
15. The compound according to any one of the previous embodiments, wherein R⁵ is selected from H, C₁₋₇ alkyl, OR⁸, or SR⁸; and wherein C₁₋₇ alkyl, OR⁸ or SR⁸ of R⁵ can form a ring with any part of X or Y, wherein the ring optionally contains a carbonyl group.
16. The compound according to any one of the previous embodiments, wherein R⁶ is selected from H, C₁₋₇ alkyl, C₂₋₇ alkenyl, C₂₋₇ alkynyl, C₃₋₇ cycloalkyl, or C₄₋₇ cycloalkenyl; or is imidazolidinone.
17. The compound according to any one of the previous embodiments, wherein R⁶ is H, C₁₋₇ alkyl, or imidazolidinone.
18. The compound according to any one of the previous embodiments, wherein the compound is of any one of Formulae (IVa), (IVb), (IVc) or (IVd):

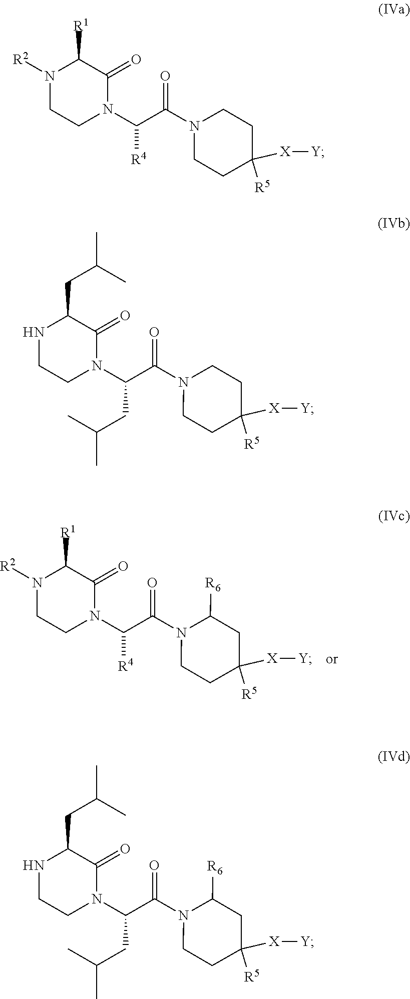

or a pharmaceutically acceptable salt, hydrate, solvate or stereoisomer thereof, wherein R¹, R², R⁴, R⁵, R⁶, X, and Y are as described herein.
19. The compound according to any one of the previous embodiments, wherein R⁸ and R¹¹ are each independently selected from H, C₁₋₇ alkyl, C₂₋₇ alkenyl, or C₃₋₇ cycloalkyl.
20. The compound according to any one of the previous embodiments, wherein R⁹ is selected from H, C₁₋₅ alkyl, halogen, C₁₋₅ alkyl-NR⁸R¹¹, C₁₋₅ alkyl-C(O)OR⁸, C₁₋₅ alkyl-C(O)NR⁸R¹¹, CN, C(O)R⁸, C(O)NR⁸R¹¹, C(O)OR⁸, or OR⁸.
21. The compound according to any one of the previous embodiments, wherein R¹⁰ and R¹² are each independently selected from H, C₁₋₇ alkyl, C₂₋₇ alkenyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen or OR⁸.
22. The compound according to any one of the previous embodiments, wherein R¹⁴ is selected from C₁₋₇ alkyl, C₃₋₇ cycloalkyl, or C₁₋₃ alkyl substituted by aryl or heteroaryl, wherein the aryl or the heteroaryl is optionally substituted by halogen, C₁₋₄ alkyl or C₃₋₅ cycloalkyl.
23. The compound according to any one of the previous embodiments, wherein R¹⁴ is selected from C₁₋₇ alkyl or C₃₋₇ cycloalkyl.
24. The compound according to any one of the previous embodiments, wherein R¹⁴ is C₁₋₇ alkyl.
25. The compound according to any one of the previous embodiments, wherein each R¹⁵ is independently selected from H, C₁₋₇ alkyl, or C₃₋₇ cycloalkyl.
26. The compound according to any one of the previous embodiments, wherein each R¹⁵ is independently selected from H, C₁₋₇ alkyl.
27. The compound according to any one of the previous embodiments, wherein X is selected from a bond, C₁₋₇ alkanediyl, —O—, C₁₋₃ alkanediyl-O—, —O—C₁₋₇ alkanediyl, C₁₋₃ alkanediyl-O—C₁₋₇ alkanediyl, C₁₋₇ heteroalkanediyl, or —S—C₁₋₇ alkanediyl; and wherein X can form a ring with any part of R⁵ or Y, wherein the ring optionally contains a carbonyl group.
28. The compound according to any one of the previous embodiments, wherein X is selected from a bond and C₁₋₇ alkanediyl, and wherein C₁₋₇ alkanediyl of X can form a ring with any part of Y.
29. The compound according to any one of the previous embodiments, wherein X is selected from a bond, —O—C₁₋₇ alkanediyl, —S—C₁₋₇ alkanediyl and C₁₋₇ alkanediyl, and wherein —O—C₁₋₇ alkanediyl, —S—C₁₋₇ alkanediyl or C₁₋₇ alkanediyl of X can form a ring with any part of R⁵, wherein the ring optionally contains a carbonyl group.
30. The compound according to any one of the previous embodiments, wherein Y is selected from H, C(O)NR¹⁰R¹², C(O)OR¹⁰, NR¹⁰R¹², CN, C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or aryl, heteroaryl wherein the aryl or heteroaryl is optionally substituted by one or more of R⁸; and wherein when Y is C(O)NR¹⁰R¹² or NR¹⁰R¹², Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.
31. The compound according to any one of the previous embodiments, wherein Y is selected from H, C(O)NR¹⁰R¹², C(O)OR¹⁰, NR¹⁰R¹², CN, C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or aryl, heteroaryl wherein the aryl or heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.
32. The compound according to any one of the previous embodiments, wherein Y is selected from C(O)NR¹⁰R¹², NR¹⁰R¹², C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or heteroaryl wherein the heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸.
33. The compound according to any one of the previous embodiments, wherein the compound is of any one of Formulae (Va), (Vb), (Vc), or (Vd):

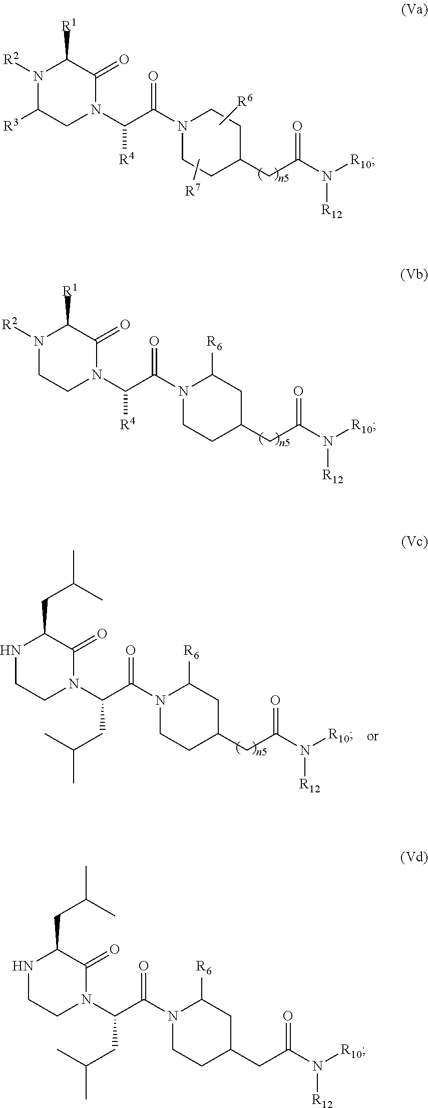

or a pharmaceutically acceptable salt, hydrate, solvate or stereoisomer thereof, wherein n5 is 0, 1, 2, 3, 4, 5, 6, or 7, preferably 1, 2, or 3, and R¹, R², R³, R⁴, R⁶, R⁷, R¹⁰ and R¹² are as described herein.
34. The compound according to any one of the previous embodiments, wherein the compound is of any one of Formulae (VIa), (VIb), (VIc), or (VId):

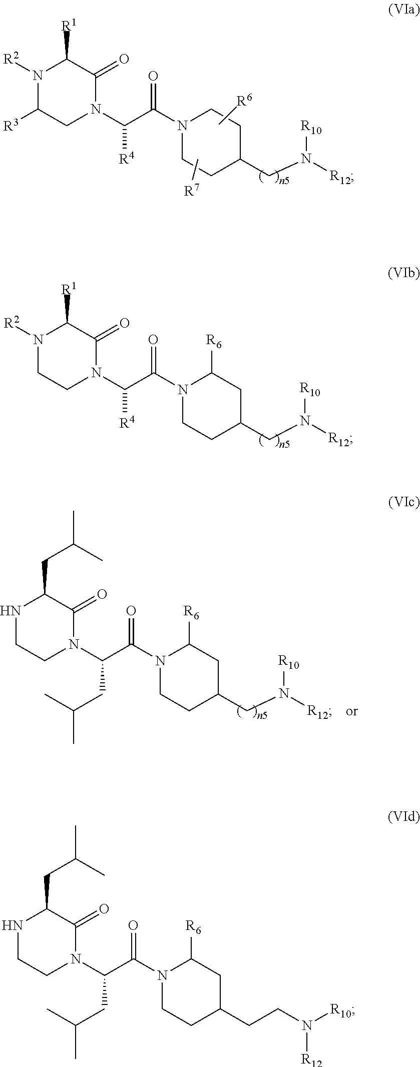

or a pharmaceutically acceptable salt, hydrate, solvate or stereoisomer thereof, wherein n5 is 0, 1, 2, 3, 4, 5, 6, or 7, preferably 1, 2, or 3, and R¹, R², R³, R⁴, R⁶, R⁷, R¹⁰ and R¹² are as described herein.
35. The compound according to any one of the previous embodiments, wherein Y is selected from NR¹⁰R¹² and C₃₋₇-cycloalkyl optionally containing a heteroatom in the ring selected from O and N wherein if the heteroatom is N it is optionally substituted by R⁸; and wherein Y can form a ring with any part of X or R⁵; with the proviso that when Y is NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸. 36. The compound according to any one of the previous embodiments, wherein R⁵ is selected from H and C₁₋₇ alkyl; wherein C₁₋₇ alkyl of R⁵ can form a ring with any part of Y;
wherein X is selected from a bond and C₁₋₇ alkanediyl, and wherein C₁₋₇ alkanediyl of X can form a ring with any part of Y;
wherein Y is selected from NR¹⁰R¹² and C₃₋₇-cycloalkyl optionally containing a heteroatom in the ring wherein the heteroatom is N and is optionally substituted by R⁸ wherein R⁸ is C₁₋₇ alkyl;
wherein Y can form a ring with any part of C₁₋₇ alkanediyl of X or with any part of C₁₋₇ alkyl of R⁵; with the proviso that when Y is NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸; and
wherein R¹⁰ and R¹² are each independently selected from H, C₁₋₇ alkyl, C₃₋₇ cycloalkyl, C₁₋₃ alkyl-aryl, all these groups optionally substituted by halogen.
   37. The compound according to any one of the previous embodiments, wherein R⁵ is selected from C₁₋₇ alkyl, OR⁸, or SR⁸; wherein C₁₋₇ alkyl, OR⁸ or SR⁸ of R⁵ can form a ring with any part of X;
wherein X is selected from —O—C₁₋₇ alkanediyl, —S—C₁₋₇ alkanediyl, or C₁₋₇ alkanediyl, and wherein —O—C₁₋₇ alkanediyl, —S—C₁₋₇ alkanediyl or C₁₋₇ alkanediyl of X can form a ring with any part of R⁵; and
wherein Y is NR¹⁰R¹² wherein R¹⁰ and R¹² can form a ring wherein the ring contains the N of NR¹⁰R¹² and optionally one further heteroatom selected from O and N, wherein if the one further heteroatom is N, it is optionally substituted by R⁸.
   38. The compound according to any one of the previous embodiments,
wherein R⁵ is OR⁸, wherein R⁸ of OR⁸ is C₁₋₇ alkyl, and wherein OR⁸ of R⁵ can form a ring with any part of X;
wherein X is —O—C₁₋₇ alkanediyl and wherein —O—C₁₋₇ alkanediyl of X can form a ring with any part of R⁵; and
wherein Y is NR¹⁰R¹² wherein R¹⁰ and R¹² can form a ring wherein the ring contains the N of NR¹⁰R¹² and four or five carbon atoms.
   39. The compound according to any one of the previous embodiments, wherein the compound is of any one of Formulae (VIIa), (VIIb), (VIIc), (VIId), (VIIe), or (VIIf):

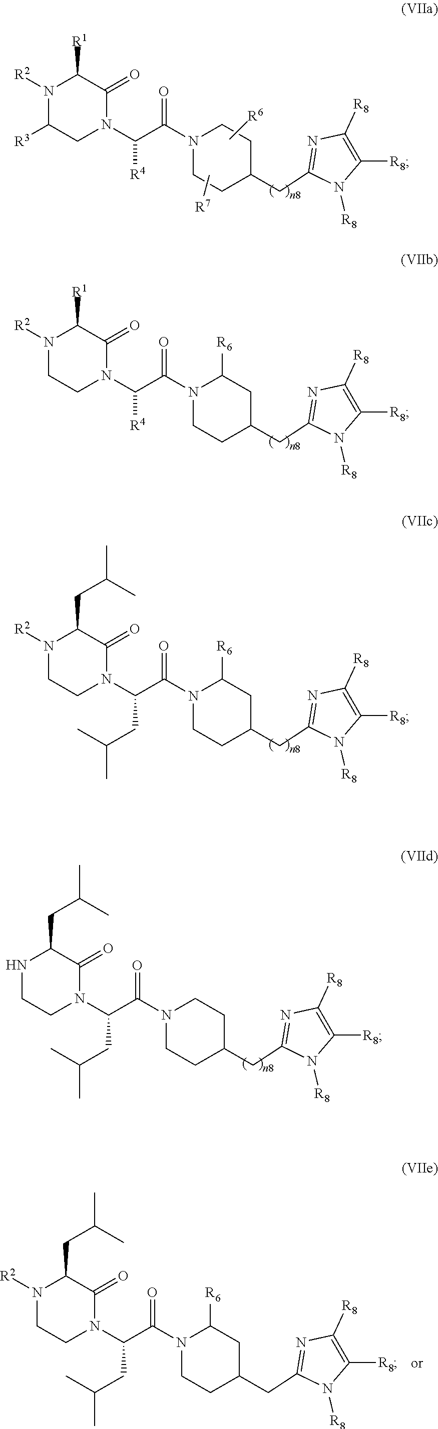

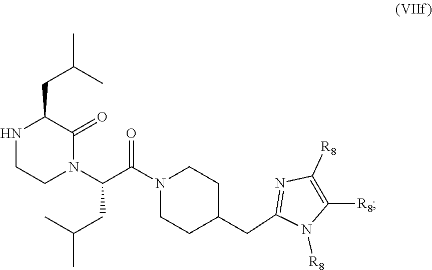

or a pharmaceutically acceptable salt, hydrate, solvate or stereoisomer thereof, wherein n8 is 0, 1, 2, 3, 4, 5, 6, or 7, preferably 1, 2, or 3, and R¹, R², R³, R⁴, R⁶, R⁷, and R⁸ are as described herein.
40. The compound according to any one of the previous embodiments,
wherein Y is aryl or heteroaryl, wherein the aryl or heteroaryl is optionally substituted by one or more of R⁸; or S-heteroaryl, wherein the S-heteroaryl is optionally substituted by one or more R¹⁴.
   41. The compound according to any one of the previous embodiments,
wherein R⁵ is selected from H and C₁₋₇ alkyl;
wherein X is selected from a bond and C₁₋₇ alkanediyl;
wherein Y is heteroaryl, wherein the heteroaryl is optionally substituted by one or more of R⁸; or S-heteroaryl, wherein the S-heteroaryl is optionally substituted by one or more R¹⁴.
   42. The compound according to any one of the previous embodiments,
wherein Y is C(O)NR¹⁰R¹²; and wherein R¹⁰ and R¹² can form a ring wherein the ring contains the N of NR¹⁰R¹² and optionally one further heteroatom selected from O and N, wherein if the one further heteroatom is N, it is optionally substituted by R⁸.
   43. The compound according to any one of the previous embodiments, wherein R⁵ is selected from H and C₁₋₇ alkyl;
wherein X is selected from a bond and C₁₋₇ alkanediyl;
wherein Y is C(O)NR¹⁰R¹²; and wherein R¹⁰ and R¹² can form a ring wherein the ring contains the N of NR¹⁰R¹² and optionally one further heteroatom selected from O and N, wherein if the one further heteroatom is N, it is optionally substituted by R⁸; and wherein R¹⁰ and R¹² are each independently selected from H, C₁₋₇ alkyl, C₂₋₇ alkenyl, C₃₋₇ cycloalkyl, C₁₋₃ alkyl-aryl.
   44. The compound according to any one of the previous embodiments, wherein Y is selected from S-aryl, O-aryl, S-heteroaryl, O-heteroaryl, wherein the S-aryl, O-aryl, S-heteroaryl, O-heteroaryl are optionally substituted by one or more R⁹ or R¹⁴.
   45. The compound according to any one of the previous embodiments,
wherein R⁵ is selected from H and C₁₋₇ alkyl;
wherein X is selected from a bond and C₁₋₇ alkanediyl,
wherein Y is selected from O-aryl and O-heteroaryl, wherein the O-aryl or O-heteroaryl is optionally substituted by one or more R⁹;
wherein R⁹ is selected from H, C₁₋₅ alkyl, halogen, C₁₋₅ alkyl-NR⁸R¹¹, C₁₋₅ alkyl-C(O)OR⁸, C₁₋₅ alkyl-C(O)NR⁸R¹¹, CN, C(O)R⁸, C(O)NR⁸R¹¹, C(O)OR⁸, and OR⁸.
   46. The compound according to any one of the previous embodiments, wherein Y is C(O)OR¹⁰.
   47. The compound according to any one of the previous embodiments, wherein
wherein R⁵ is selected from H and C₁₋₇ alkyl;
wherein X is selected from a bond and C₁₋₇ alkanediyl;
wherein Y is C(O)OR¹⁰; and
wherein R¹⁰ is selected from H, C₁₋₇ alkyl, C₂₋₇ alkenyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by OR₈.
   48. The compound according to any one of the previous embodiments, wherein Y is H.
   49. The compound according to any one of the previous embodiments, wherein
wherein R⁵ is C₁₋₇ alkyl;
wherein X is a bond; and
wherein Y is H.
   50. The compound according to any one of the previous embodiments, wherein Y is CN.
   51. The compound according to any one of the previous embodiments, wherein
R⁵ is H;
X is C₁₋₇ alkanediyl; and
Y is CN.
   52. The compound according to any one of the previous embodiments, wherein the compound is of any one of Formulae (VIIIa), (VIIIb), (VIIIc), (VIIId), (VIIIe), (VIIIf), (VIIIg), (VIIIh), (VIIIi), (VIIIj), (VIIIk), (VIIIl):

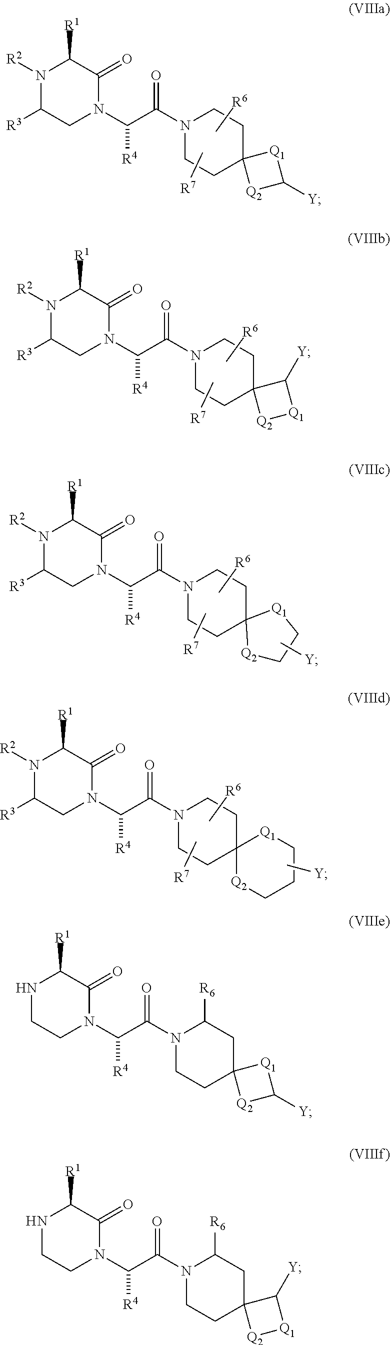

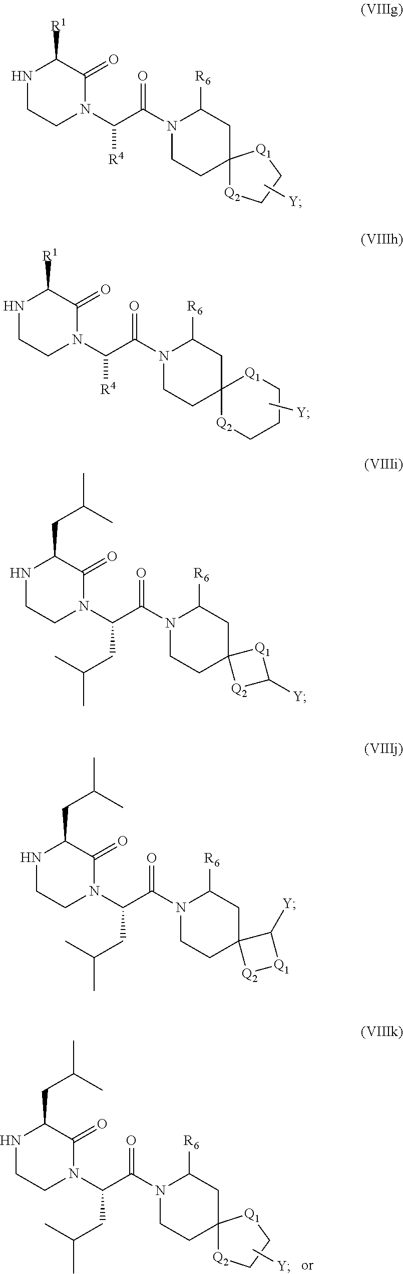

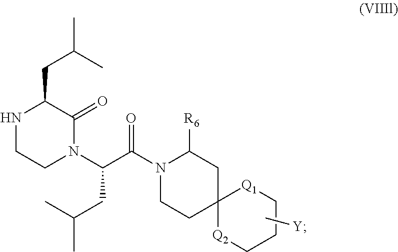

or a pharmaceutically acceptable salt, hydrate, solvate, or stereoisomer thereof, wherein Q₁ and Q₂ are each independently O, S, NR⁸, or CR⁸, and R¹, R², R³, R⁴, R⁶, R⁷, R⁸, and Y are as described herein.
53. The compound according to any one of the previous embodiments, wherein the compound is of any one of Formulae (IXa), (IXb), (IXc), or (IXd):

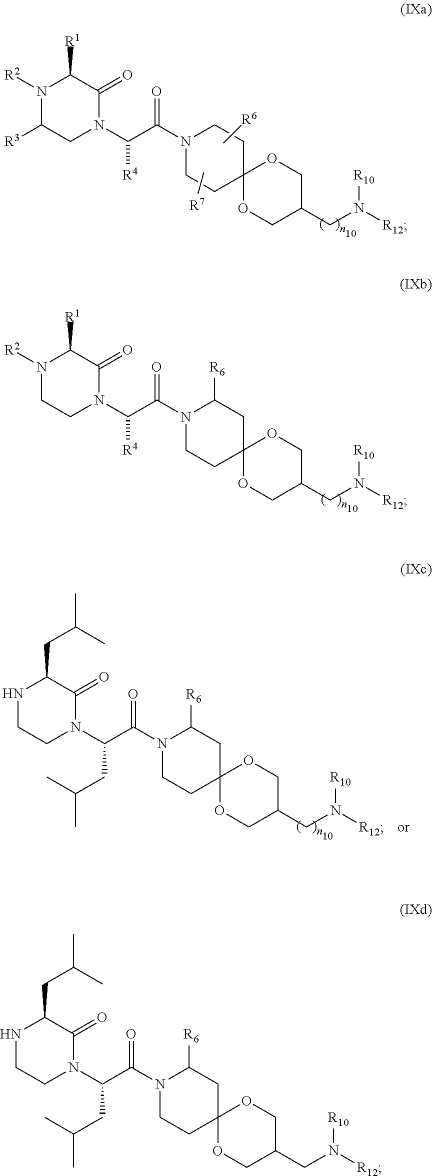

or a pharmaceutically acceptable salt, hydrate, solvate, or stereoisomer thereof, wherein n10 is 0, 1, 2, 3, 4, 5, 6, or 7, preferably 1, 2, or 3, and R¹, R², R³, R⁴, R⁶, R⁷, R¹⁰, R¹² and Y are as described herein.
54. A compound selected from the group consisting of:

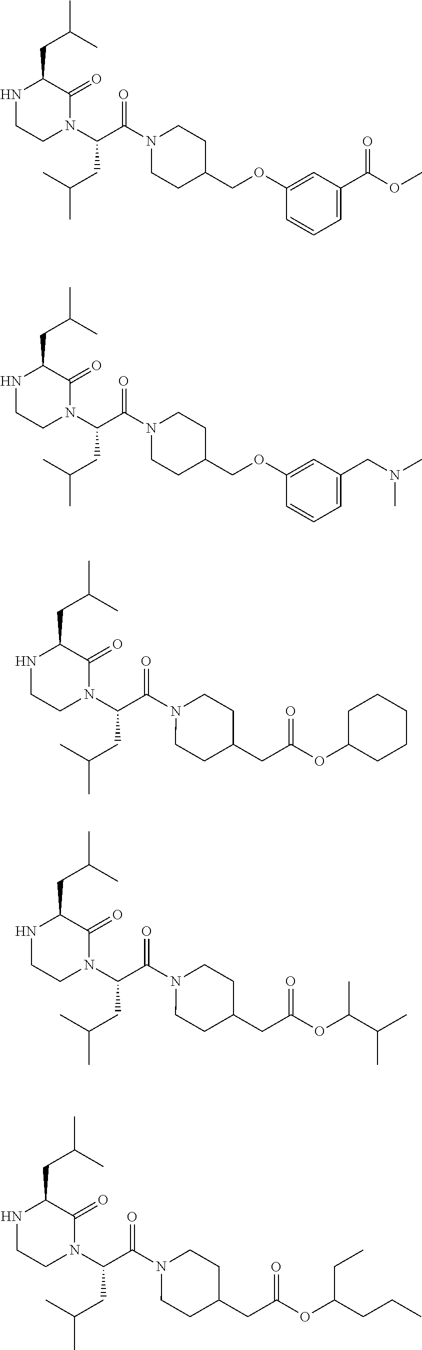

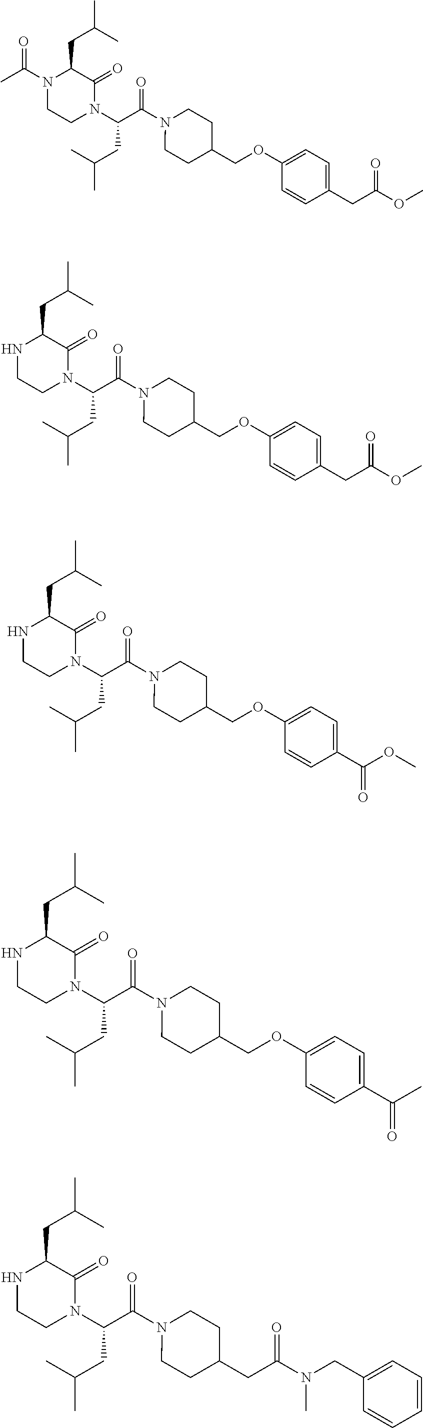 [SMILES: COC(=O)Cc1ccc(OCC2CCN(C(=O)[C@H](CC(C)C)N3CCN(C(C)=O)[C@@H](CC(C)C)C3=O)CC2)cc1 ; COC(=O)Cc1ccc(OCC2CCN(C(=O)[C@H](CC(C)C)N3CCN[C@@H](CC(C)C)C3=O)CC2)cc1 ; COC(=O)c1ccc(OCC2CCN(C(=O)[C@H](CC(C)C)N3CCN[C@@H](CC(C)C)C3=O)CC2)cc1 ; CC(=O)c1ccc(OCC2CCN(C(=O)[C@H](CC(C)C)N3CCN[C@@H](CC(C)C)C3=O)CC2)cc1 ; CC(C)C[C@@H]1NCCN([C@@H](CC(C)C)C(=O)N2CCC(CC(=O)N(C)Cc3ccccc3)CC2)C1=O]

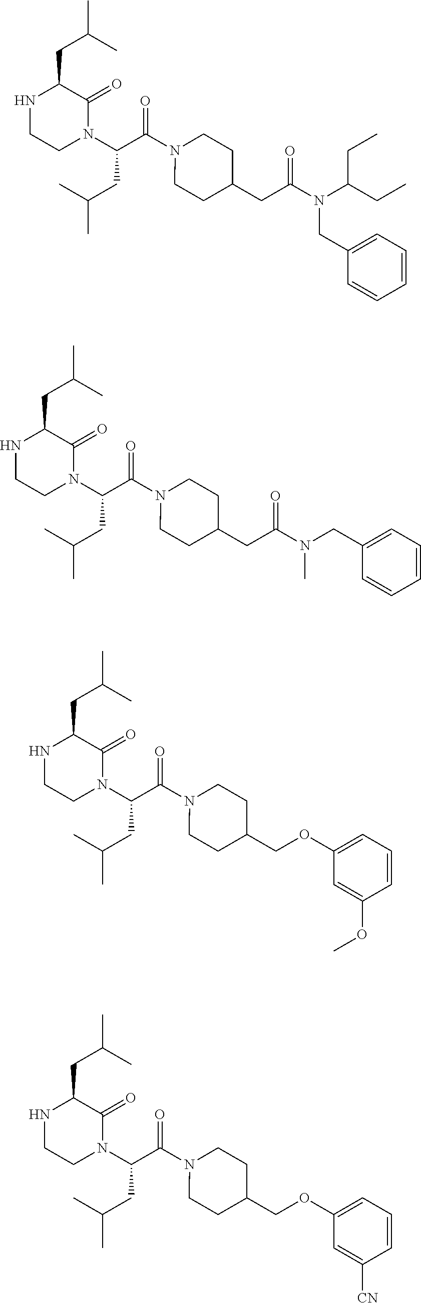 [SMILES: CCC(CC)N(Cc1ccccc1)C(=O)CC1CCN(C(=O)[C@H](CC(C)C)N2CCN[C@@H](CC(C)C)C2=O)CC1 ; CC(C)C[C@@H]1NCCN([C@@H](CC(C)C)C(=O)N2CCC(CC(=O)N(C)Cc3ccccc3)CC2)C1=O ; COc1cccc(OCC2CCN(C(=O)[C@H](CC(C)C)N3CCN[C@@H](CC(C)C)C3=O)CC2)c1 ; CC(C)C[C@@H]1NCCN([C@@H](CC(C)C)C(=O)N2CCC(COc3cccc(C#N)c3)CC2)C1=O]

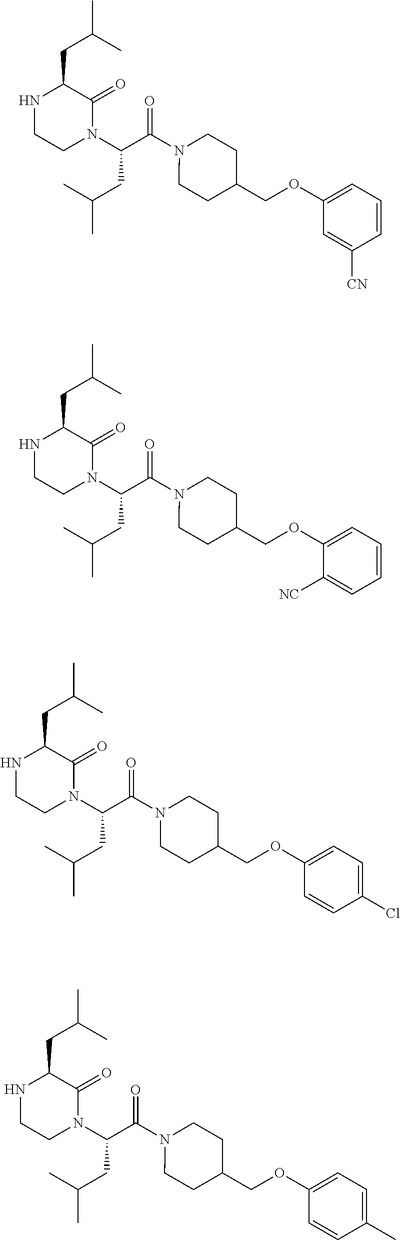 [SMILES: CC(C)C[C@@H]1NCCN([C@@H](CC(C)C)C(=O)N2CCC(COc3cccc(C#N)c3)CC2)C1=O ; CC(C)C[C@@H]1NCCN([C@@H](CC(C)C)C(=O)N2CCC(COc3ccccc3C#N)CC2)C1=O ; CC(C)C[C@@H]1NCCN([C@@H](CC(C)C)C(=O)N2CCC(COc3ccc(Cl)cc3)CC2)C1=O ; Cc1ccc(OCC2CCN(C(=O)[C@H](CC(C)C)N3CCN[C@@H](CC(C)C)C3=O)CC2)cc1]

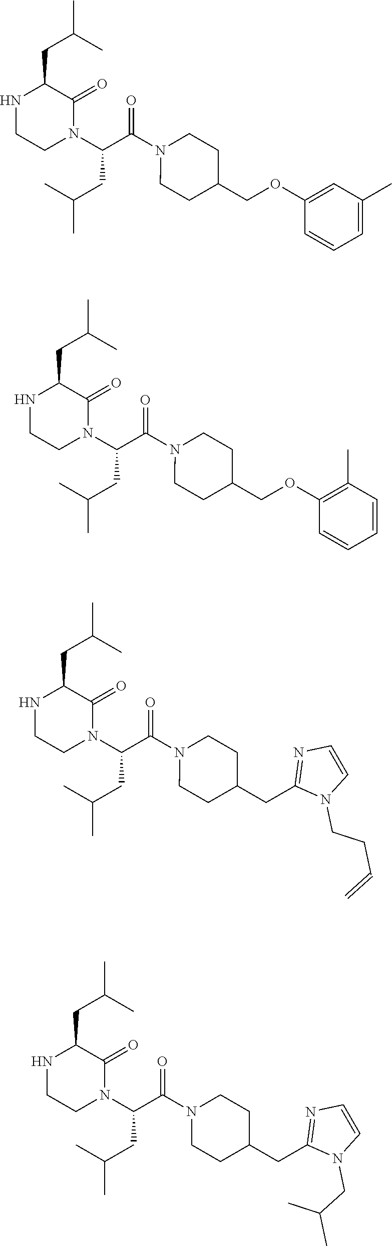 [SMILES: Cc1cccc(OCC2CCN(C(=O)[C@H](CC(C)C)N3CCN[C@@H](CC(C)C)C3=O)CC2)c1 ; Cc1ccccc1OCC1CCN(C(=O)[C@H](CC(C)C)N2CCN[C@@H](CC(C)C)C2=O)CC1 ; C=CCCn1ccnc1CC1CCN(C(=O)[C@H](CC(C)C)N2CCN[C@@H](CC(C)C)C2=O)CC1 ; CC(C)C[C@@H]1NCCN([C@@H](CC(C)C)C(=O)N2CCC(Cc3nccn3CC(C)C)CC2)C1=O]

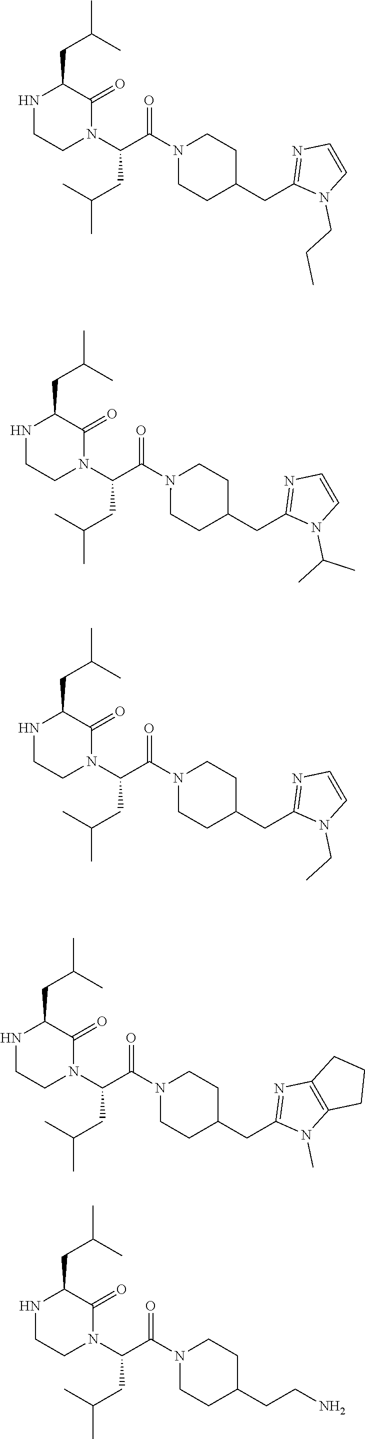

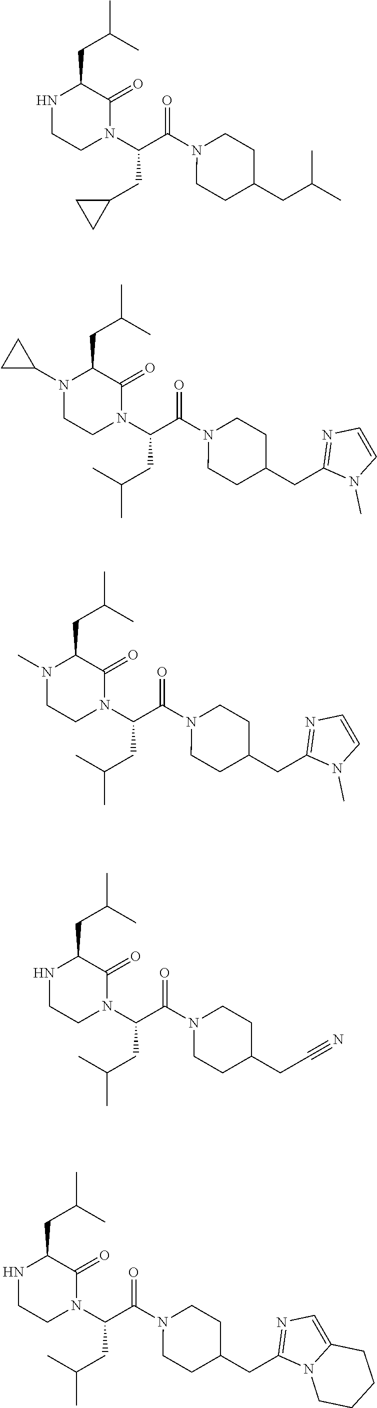

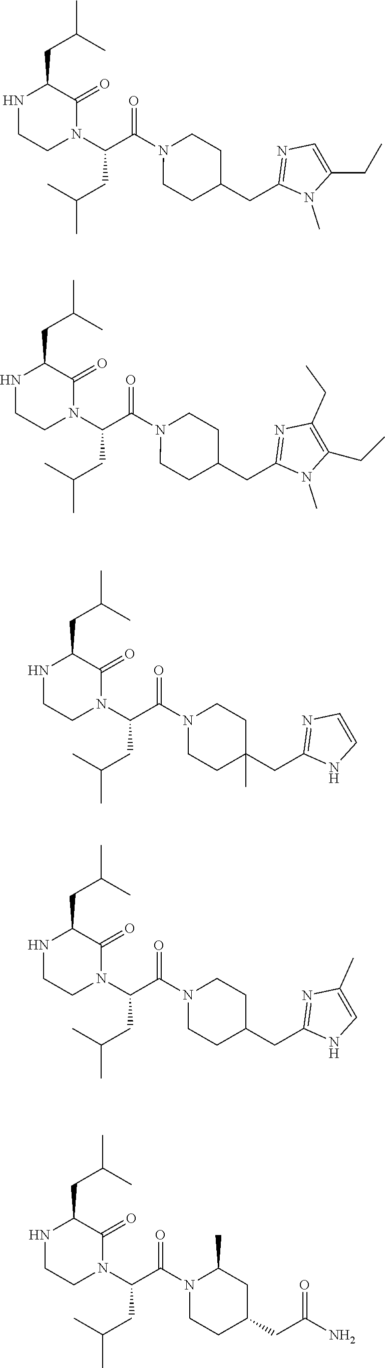

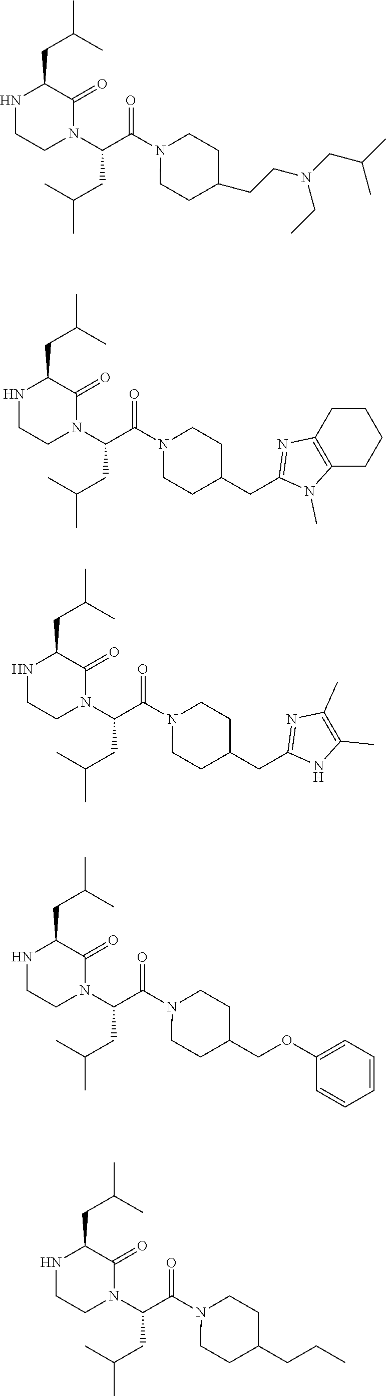

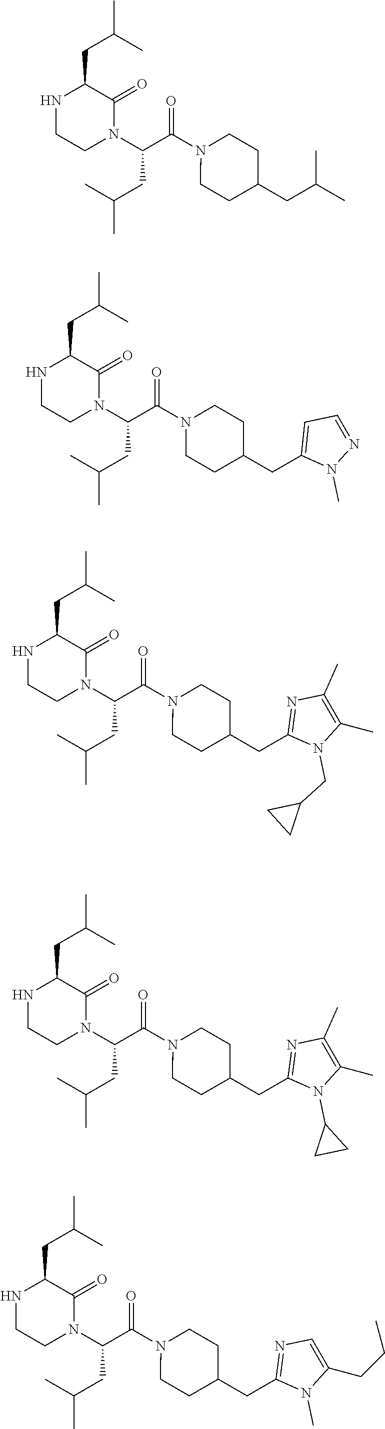

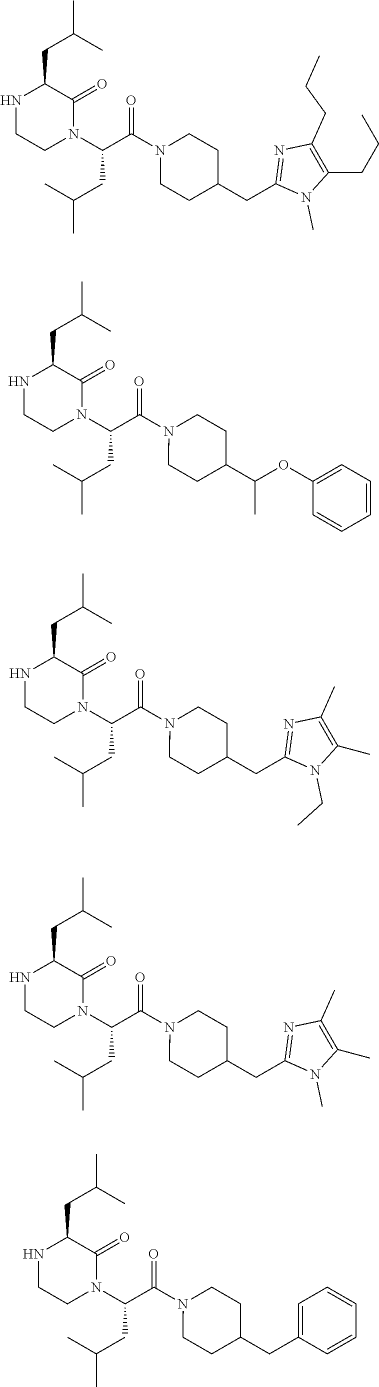

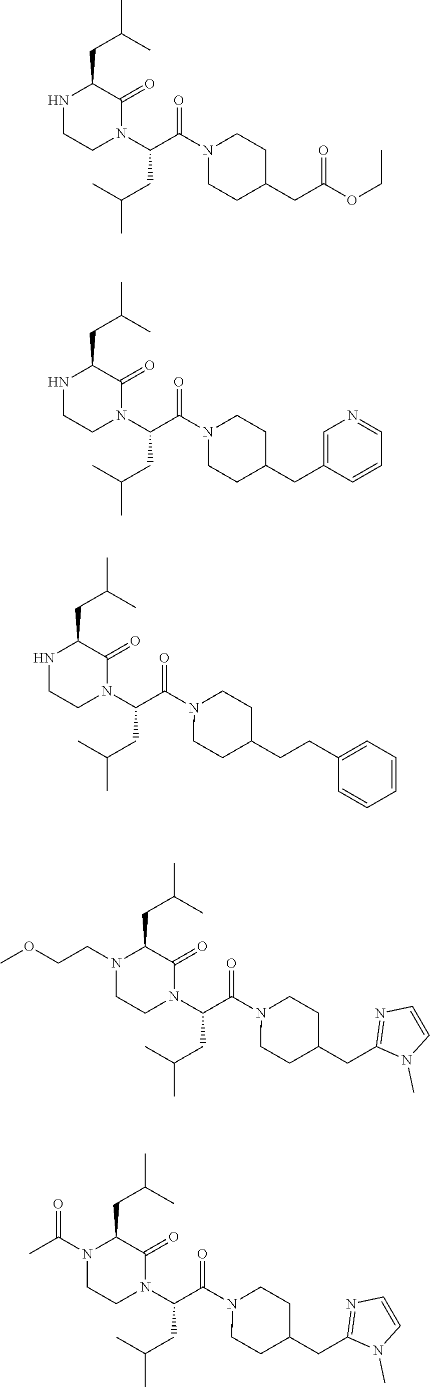

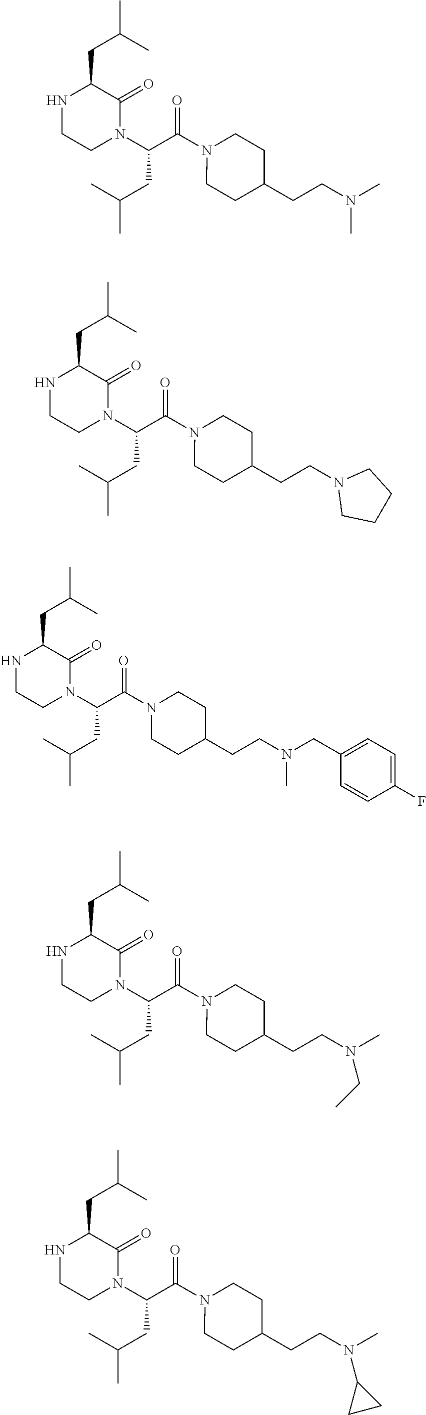 [SMILES: CC(C)C[C@@H]1NCCN([C@@H](CC(C)C)C(=O)N2CCC(CCN(C)C)CC2)C1=O ; CC(C)C[C@@H]1NCCN([C@@H](CC(C)C)C(=O)N2CCC(CCN3CCCC3)CC2)C1=O ; CC(C)C[C@@H]1NCCN([C@@H](CC(C)C)C(=O)N2CCC(CCN(C)Cc3ccc(F)cc3)CC2)C1=O ; CCN(C)CCC1CCN(C(=O)[C@H](CC(C)C)N2CCN[C@@H](CC(C)C)C2=O)CC1 ; CC(C)C[C@@H]1NCCN([C@@H](CC(C)C)C(=O)N2CCC(CCN(C)C3CC3)CC2)C1=O]

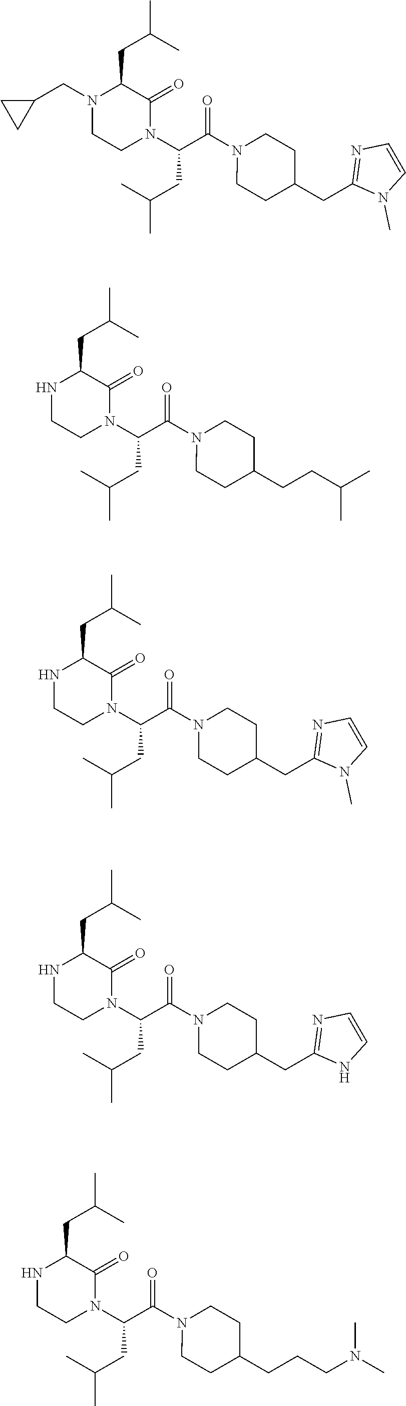 [SMILES: CC(C)C[C@H]1C(=O)N([C@@H](CC(C)C)C(=O)N2CCC(Cc3nccn3C)CC2)CCN1CC1CC1 ; CC(C)CCC1CCN(C(=O)[C@H](CC(C)C)N2CCN[C@@H](CC(C)C)C2=O)CC1 ; CC(C)C[C@@H]1NCCN([C@@H](CC(C)C)C(=O)N2CCC(Cc3nccn3C)CC2)C1=O ; CC(C)C[C@@H]1NCCN([C@@H](CC(C)C)C(=O)N2CCC(Cc3ncc[nH]3)CC2)C1=O ; CC(C)C[C@@H]1NCCN([C@@H](CC(C)C)C(=O)N2CCC(CCCN(C)C)CC2)C1=O]

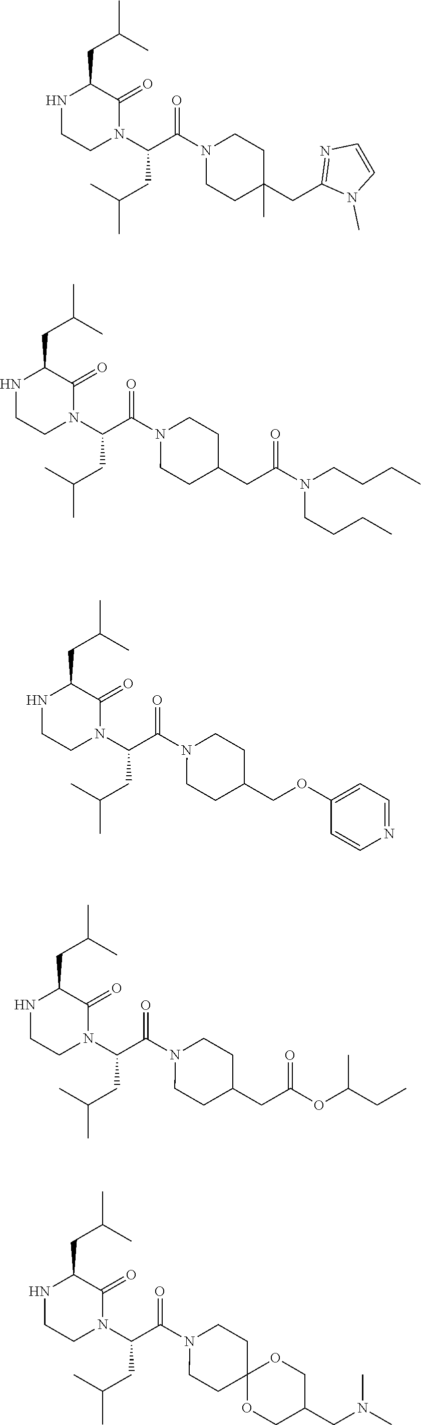 [SMILES: CC(C)C[C@@H]1NCCN([C@@H](CC(C)C)C(=O)N2CCC(C)(Cc3nccn3C)CC2)C1=O ; CCCCN(CCCC)C(=O)CC1CCN(C(=O)[C@H](CC(C)C)N2CCN[C@@H](CC(C)C)C2=O)CC1 ; CC(C)C[C@@H]1NCCN([C@@H](CC(C)C)C(=O)N2CCC(COc3ccncc3)CC2)C1=O ; CCC(C)OC(=O)CC1CCN(C(=O)[C@H](CC(C)C)N2CCN[C@@H](CC(C)C)C2=O)CC1 ; CC(C)C[C@@H]1NCCN([C@@H](CC(C)C)C(=O)N2CCC3(CC2)OCC(CN(C)C)CO3)C1=O]

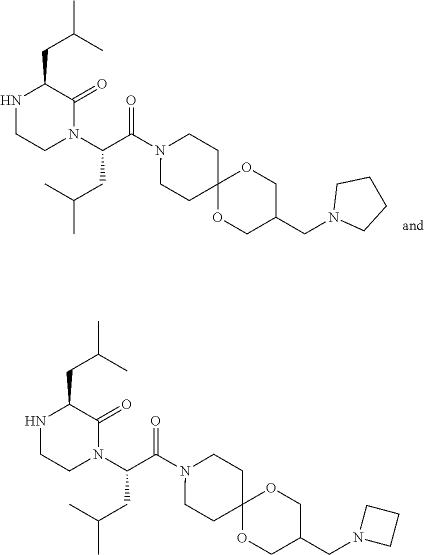 [SMILES: CC(C)C[C@@H]1NCCN([C@@H](CC(C)C)C(=O)N2CCC3(CC2)OCC(CN2CCCC2)CO3)C1=O ; CC(C)C[C@@H]1NCCN([C@@H](CC(C)C)C(=O)N2CCC3(CC2)OCC(CN2CCC2)CO3)C1=O]

55. A compound selected from the group consisting of:

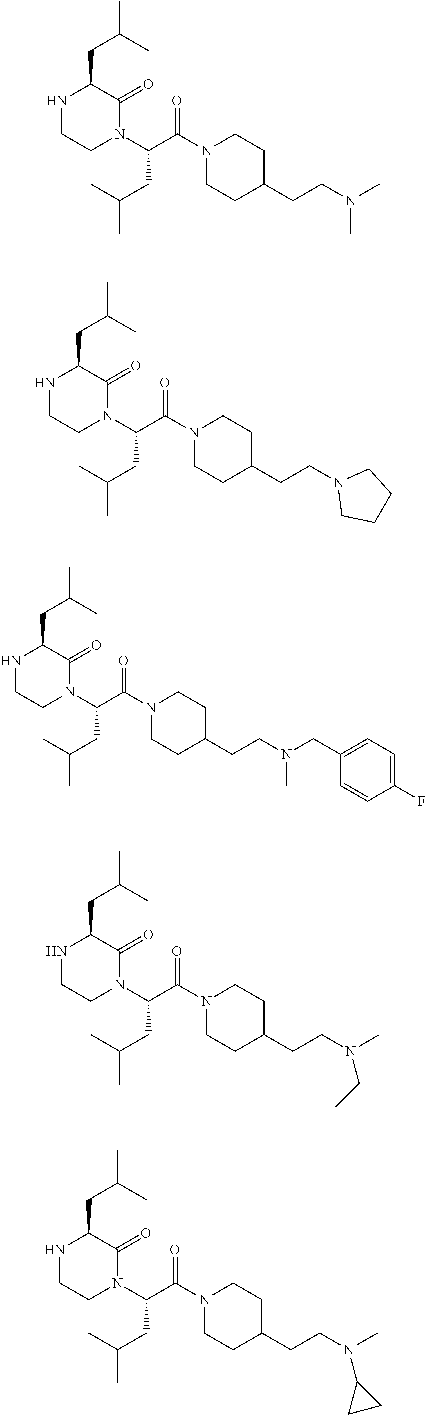 [SMILES: CC(C)C[C@@H]1NCCN([C@@H](CC(C)C)C(=O)N2CCC(CCN(C)C)CC2)C1=O ; CC(C)C[C@@H]1NCCN([C@@H](CC(C)C)C(=O)N2CCC(CCN3CCCC3)CC2)C1=O ; CC(C)C[C@@H]1NCCN([C@@H](CC(C)C)C(=O)N2CCC(CCN(C)Cc3ccc(F)cc3)CC2)C1=O ; CCN(C)CCC1CCN(C(=O)[C@H](CC(C)C)N2CCN[C@@H](CC(C)C)C2=O)CC1 ; CC(C)C[C@@H]1NCCN([C@@H](CC(C)C)C(=O)N2CCC(CCN(C)C3CC3)CC2)C1=O]

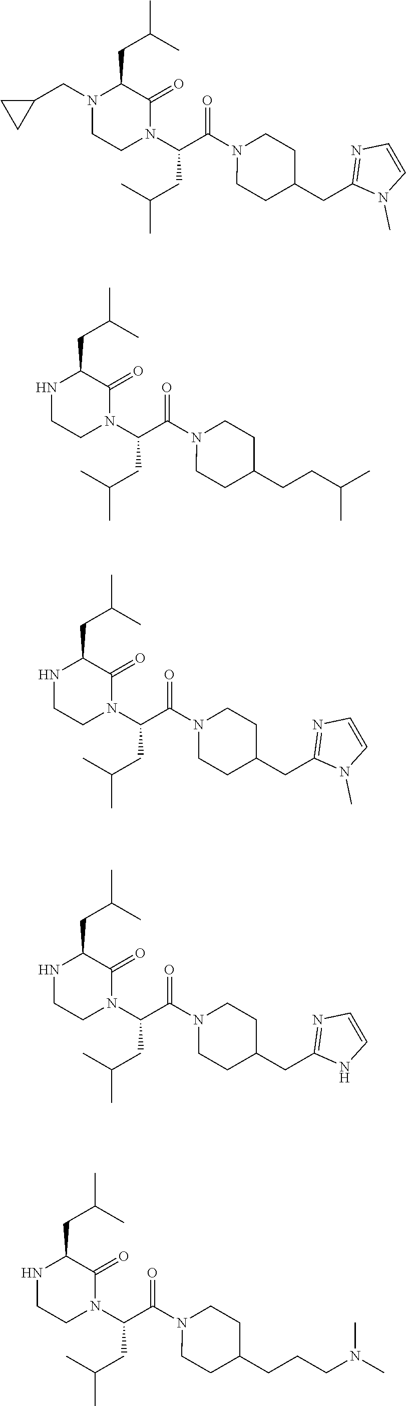 [SMILES: CC(C)C[C@H]1C(=O)N([C@@H](CC(C)C)C(=O)N2CCC(Cc3nccn3C)CC2)CCN1CC1CC1 ; CC(C)CCC1CCN(C(=O)[C@H](CC(C)C)N2CCN[C@@H](CC(C)C)C2=O)CC1 ; CC(C)C[C@@H]1NCCN([C@@H](CC(C)C)C(=O)N2CCC(Cc3nccn3C)CC2)C1=O ; CC(C)C[C@@H]1NCCN([C@@H](CC(C)C)C(=O)N2CCC(Cc3ncc[nH]3)CC2)C1=O ; CC(C)C[C@@H]1NCCN([C@@H](CC(C)C)C(=O)N2CCC(CCCN(C)C)CC2)C1=O]

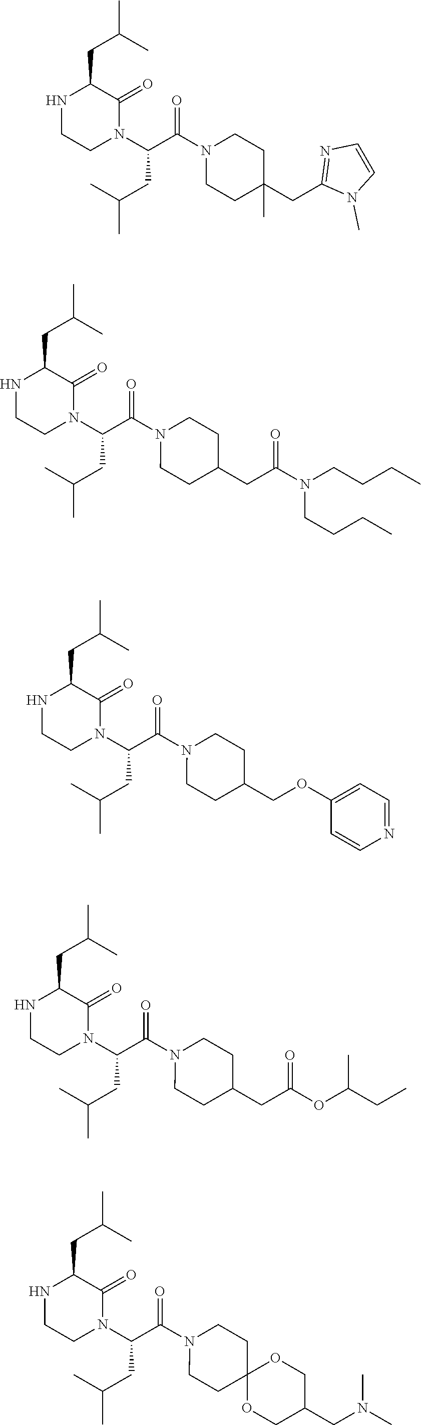 [SMILES: CC(C)C[C@@H]1NCCN([C@@H](CC(C)C)C(=O)N2CCC(C)(Cc3nccn3C)CC2)C1=O ; CCCCN(CCCC)C(=O)CC1CCN(C(=O)[C@H](CC(C)C)N2CCN[C@@H](CC(C)C)C2=O)CC1 ; CC(C)C[C@@H]1NCCN([C@@H](CC(C)C)C(=O)N2CCC(COc3ccncc3)CC2)C1=O ; CCC(C)OC(=O)CC1CCN(C(=O)[C@H](CC(C)C)N2CCN[C@@H](CC(C)C)C2=O)CC1 ; CC(C)C[C@@H]1NCCN([C@@H](CC(C)C)C(=O)N2CCC3(CC2)OCC(CN(C)C)CO3)C1=O]

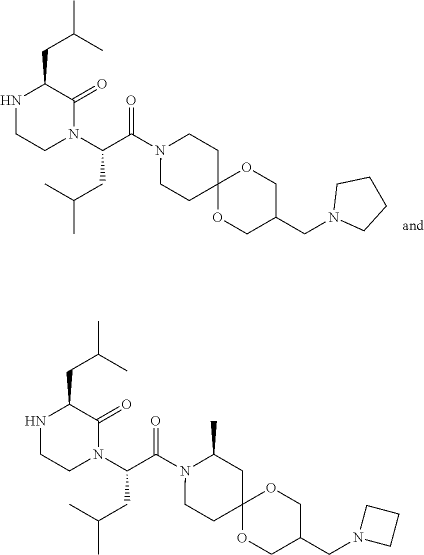 [SMILES: CC(C)C[C@@H]1NCCN([C@@H](CC(C)C)C(=O)N2CCC3(CC2)OCC(CN2CCCC2)CO3)C1=O ; CC(C)C[C@@H]1NCCN([C@@H](CC(C)C)C(=O)N2CCC3(C[C@@H]2C)OCC(CN2CCC2)CO3)C1=O]

56. The compound according to any one of the previous embodiments, wherein the compound is selected from a compound of any one of Table 2 or Table 3.
57. A pharmaceutical composition comprising a compound according to any one of the previous embodiments and a pharmaceutically acceptable diluent, excipient or carrier.
58. The pharmaceutical composition according to embodiment 57, further comprising an additional pharmaceutically active agent.
59. The pharmaceutical composition according to embodiment 58, wherein the additional pharmaceutically active agent comprises an additional cancer therapy.
60. The compound according to any one of embodiments 1-56 or the pharmaceutical composition according to embodiment 57 or 58 for use as a medicament.
61. The compound according to any one of embodiments 1-56 or the or the pharmaceutical composition according to embodiment 57 or 58 for use in a method for preventing or treating cancer in a subject in need thereof.
62. The compound according to any one of embodiments 1-56 or the pharmaceutical composition according to embodiment 57 or 58 for the use in the manufacture of a medicament for the treatment of cancer in a subject in need thereof.
63. A method of treating cancer in a subject in need thereof, comprising administering to the subject a therapeutically effective amount of a composition comprising the compound of any one of embodiments 1-56 or the pharmaceutical composition of embodiment 57 or 58.
64. The method or composition for use according to any one of embodiments 61-63, wherein the cancer comprises a solid tumor or a liquid tumor.
65. The method or composition for use according to embodiment 64, wherein the solid tumor is a primary tumor or a metastatic tumor.
66. The method or composition for use according to embodiment 64, wherein the solid tumor is a carcinoma, a sarcoma, a myeloma, a germ cell tumor, a carcinoid tumor, a neuroendocrine tumor or a tumor of mixed type.
67. The method or composition for use according to embodiment 64, wherein the cancer comprises a lymphoma, a leukemia, a brain cancer, a nervous system cancer, a breast cancer, a cervical cancer, an ovarian cancer, a colorectal cancer, a stomach cancer, a gastric cancer, a kidney cancer, a liver cancer, a lung cancer, an oesophageal cancer, a pancreatic cancer, a prostate cancer, a colon cancer, a skin cancer or a head-and-neck cancer.
68. The method or composition for use according to embodiment 64, wherein the liquid tumor is a leukemia or a lymphoma.
69. The method of composition for use according to any one of embodiments 61-66, wherein the cancer is Stage I, Stage IIA, Stage IIB, Stage IIIA, Stage IIIB, Stage IIIC, or Stage IV cancer.
70. The method or composition for use according to any one of embodiments 61-69, wherein the subject is a mouse, a rat, a rabbit, a non-human primate or a human.
71. The method or composition for use according to embodiment 70, wherein the human is a child, an adolescent or an adult.
72. The method of composition for use according to any one of embodiments 61-71, wherein the compound or pharmaceutical composition is suitable for oral administration.
73. The method of composition for use according to any one of embodiments 61-71, wherein the compound or pharmaceutical composition is suitable for parenteral administration.
74. The method or composition for use according to embodiment 73, wherein the parenteral administration comprises subcutaneous administration, intravenous injection, intravenous infusion, intraperitoneal injection, intramuscular injection or intratumoral injection.
75. The method or composition for use according to any one of embodiments 61-74, wherein the method or use of the composition further comprises at least one additional cancer therapy.
76. The method or composition for use according to embodiment 75, wherein the at least one additional cancer therapy comprises a standard of care for the cancer.
77. The method or composition for use according to embodiment 75 or 76, wherein the at least one additional cancer therapy comprises surgical resection of the cancer, radiation therapy, or a combination thereof.
78. The method or composition for use according to embodiment 75, wherein the at least one additional cancer therapy comprises administration of at least one additional cancer therapeutic agent.
79. The method or composition for use according to embodiment 78, wherein the administration comprises simultaneous administration of the compound or pharmaceutical composition and the at least one additional cancer therapeutic agent.
80. The method or composition for use according to embodiment 79, wherein the compound or pharmaceutical composition and the at least one additional cancer therapeutic agent are in the same composition.
81. The method or composition for use according to embodiment 78, wherein the administration comprises administration in temporal proximity of the compound or pharmaceutical composition and the at least one additional cancer therapeutic agent.
82. The method or composition for use according to embodiment 78, wherein the administration comprises sequential administration of the compound or pharmaceutical composition and the at least one additional cancer therapeutic agent.
83. The method or composition for use according to any one of embodiments 78-82, wherein the at least one additional cancer therapeutic agent comprises a chemotherapeutic agent.
84. The method or composition for use according to embodiment 83, wherein the chemotherapeutic agent comprises a platinum compound, an alkylating agent, an antitumor antibiotic, a taxane, an antimetabolite, a nucleoside analog, a topoisomerase inhibitor, a hypomethylating agent, a proteasome inhibitor, an epipodophyllotoxin, a DNA synthesis inhibitor, a vinca alkaloid, a tyrosine kinase inhibitor, a nitrosourea, hexamethylmelamine, mitotane, an angiogenesis inhibitor, a steroid, a hormonal agent, an aromatase inhibitor, arsenic trioxide, tretinoin, a nonselective cyclooxygenase inhibitor, a selective cyclooxygenase-2 (COX-2) inhibitors, or a combination thereof.
85. The method or composition for use according to any one of embodiments 78-82, wherein the at least one additional cancer therapeutic agent comprises a biological agent.
86. The method or composition for use according to embodiment 85, wherein the biological agent comprises an antibody therapy, an adoptive cell therapy, an enzyme, a cytokine, a growth factor, an inhibitor of a growth factor, a gene therapy a cancer vaccine or a combination thereof.
87. The method or composition for use according to embodiment 86, wherein the antibody therapy comprises ituximab, cetuximab, obinutuzumab, ofatumumab, ibritumomab, brentuximab, bevacizumab, panitumumab, pembrolizumab, tositumomab, trastuzumab, alemtuzumab, gemtuzumab ozogamicin, bevacizumab, catumaxomab, denosumab, obinutuzumab, ofatumumab, ramucirumab, pertuzumab, ipilimumab, nivolumab, nimotuzumab, lambrolizumab, pidilizumab, siltuximab, tremelimumab.
88. The method or composition for use according to embodiment 86, wherein the adoptive cell therapy comprises a chimeric antigen receptor T cell (CAR-T) therapy.
89. The method or composition for use according to embodiment 88, wherein the adoptive cell therapy is autologous or allogeneic.
90. The method or composition for use according to any one of embodiments 78-82, wherein the at least one additional cancer therapeutic agent comprises an immune checkpoint inhibitor.
91. The method or composition for use according to embodiment 90, wherein the immune checkpoint inhibitor comprises nivolumab, pembrolizumab, atezolizumab, avelumab, durvalumab or ipilimumab.
92. The method or composition for use according to embodiment 86, wherein the antibody therapy comprises a VEGFA antibody.
93. The method or composition for use according to embodiment 92, wherein the VEGFA antibody comprises bevacizumab (Avastin®).
94. The method or composition for use according to any one of embodiments 61-93, wherein the method or use of the composition alleviates a sign or a symptom of the cancer.
95. The method or composition for use according to embodiment 94, wherein alleviating a sign or a symptom of the cancer comprises a reduction in tumor volume, a reduction in tumor size, a reduction in tumor number, a decrease in the rate of growth of a tumor or a combination thereof.
96. A kit, comprising the compound according to any one of embodiments 1-56 or the pharmaceutical composition according to embodiment 57 or 58 and instructions for use in treating cancer in a subject in need thereof.
97. The kit according to embodiment 96, further comprising at least one additional cancer therapeutic agent.
98. The kit according to embodiment 97, wherein the at least one additional cancer therapeutic agent comprises a chemotherapeutic agent.
99. The kit according to embodiment 98, wherein the chemotherapeutic agent comprises a platinum compound, an alkylating agent, an antitumor antibiotic, a taxane, an antimetabolite, a nucleoside analog, a topoisomerase inhibitor, a hypomethylating agent, a proteasome inhibitor, an epipodophyllotoxin, a DNA synthesis inhibitor, a vinca alkaloid, a tyrosine kinase inhibitor, a nitrosourea, hexamethylmelamine, mitotane, an angiogenesis inhibitor, a steroid, a hormonal agent, an aromatase inhibitor, arsenic trioxide, tretinoin, a nonselective cyclooxygenase inhibitor, a selective cyclooxygenase-2 (COX-2) inhibitors, or a combination thereof.
100. The kit of embodiment according to embodiment 97, wherein the at least one additional cancer therapeutic agent comprises a biological agent.
101. The kit according to embodiment 100, wherein biological agent comprises an antibody therapy, an adoptive cell therapy, an enzyme, a cytokine, a growth factor, an inhibitor of a growth factor, a gene therapy a cancer vaccine or a combination thereof.
102. The kit according to embodiment 97, wherein the at least one additional cancer therapeutic agent comprises an immune checkpoint inhibitor.
103. The kit according to embodiment 102, wherein the immune checkpoint inhibitor comprises nivolumab, pembrolizumab, atezolizumab, avelumab, durvalumab or ipilimumab.

### EXAMPLES

Abbreviations used in the following examples and elsewhere herein are:
DCC: dicyclohexylcarbodiimide
DCM: dichloromethane
DMF: dimethylformamide
DMP: Dess-Martin periodinane; 1,1,1-Triacetoxy-1,1-dihydro-1,2-benziodoxol-3(1H)-one
DMSO: dimethylsulfoxide
HATU: 1-[bis(dimethylamino)methylene]-1H-1,2,3-triazolo[4,5-b]pyridinium 3-oxid hexafluorophosphate
MS: mass spectrum
NMP: N-methyl-2-pyrrolidone
NMR: nuclear magnetic resonance
Nosyl: 2-nitrosulfonyl
TBDMS: t-butyldimethylsilyl
tBOC or BOC: t-butyloxycarbonyl
TFA: trifluoroacetic acid
THF: tetrahydrofuran
THP: 2-tetrahydropyranyl

The compounds have been prepared in accordance to the following schemes/methods. However, other methods are known for the synthesis.

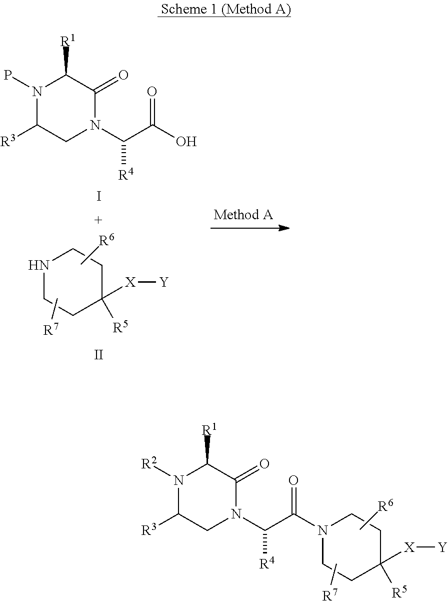

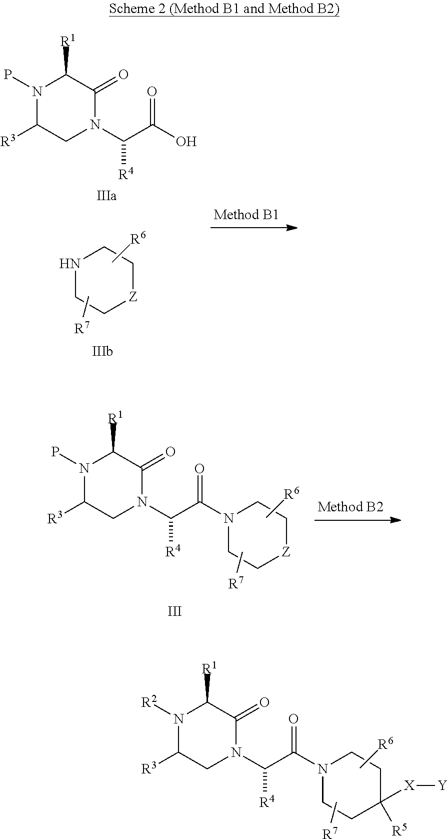

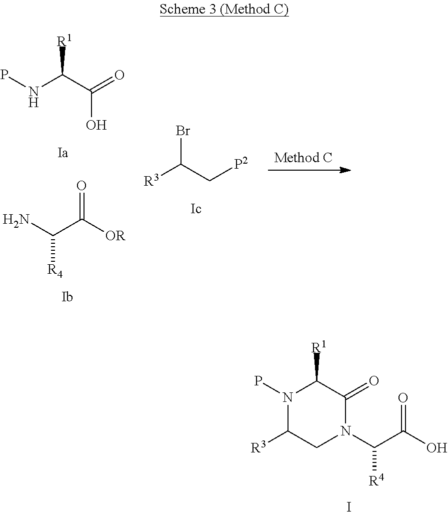

### Synthesis of I, (S)-2-[(S)-3-isobutyl-4-(o-nitrophenylsulfonyl)-2-oxo-1-piperazinyl]-4-methylvaleric acid according to synthetic method C (Scheme 3)

### Synthesis of Ia (S)-4-methyl-2-(o-nitrophenylsulfonylamino)valeric acid

To a solution of L-leucine (1 eq.) and N,N-diisopropylethylamine (3.2 eq.) in a water/THF solvent mixture cooled at 0° C. is added o-(chlorosulfonyl)nitrobenzene (1.3 eq.). The solution is allowed to warm up to room temperature and stirred overnight. The residue is acidified, extracted and concentrated to yield the title compound as an orange solid.

¹H NMR (300 MHz, CD₃OD), δ (ppm): 8.16-8.03 (m, 1H), 7.90-7.73 (m, 3H), 4.07 (dd, 1H), 1.88-1.72 (m, 1H), 1.66-1.51 (m, 2H), 0.94 (d, 3H), 0.88 (d, 3H)

MS⁻: 315 (M−H)

### Synthesis of methyl (S)-2-[(S)-4-methyl-2-(o-nitrophenylsulfonylamino)valerylamino]-4-methylvalerate

To a solution of Ia ((S)-4-methyl-2-(o-nitrophenylsulfonylamino)valeric acid) (1 eq.), Ib (methyl (S)-2-amino-4-methylvalerate hydrochloride) (1.1 eq.) and N,N-diisopropylethylamine (3 eq.) in DMF is added HATU reagent (1.1 eq.). The solution is stirred at room temperature overnight. After aqueous work-up, the organic residue is concentrated and re-crystallized to yield the title compound as a colourless solid.

¹H NMR (300 MHz, CDCl₃), δ (ppm): 8.14-8.09 (m, 1H), 7.94-7.88 (m, 1H), 7.78-7.70 (m, 2H), 6.48 (d, 1H), 6.15 (d, 1H), 4.45-4.35 (m, 1H), 4.06-3.97 (m, 1H), 3.68 (s, 3H), 1.80-1.37 (m, 8H), 0.90-0.85 (m, 6H), 0.82-0.78 (m, 6H)

MS⁻: 442 (M−H)

### Synthesis of methyl (S)-2-[(S)-3-isobutyl-4-(o-nitrophenylsulfonyl)-2-oxo-1-piperazinyl]-4-methylvalerate

To a solution of methyl (S)-2-[(S)-4-methyl-2-(o-nitrophenylsulfonylamino)valerylamino]-4-methylvalerate (1 eq.) and Ic (1,2-dibromoethane) (4 eq.) in DMF is added potassium carbonate (4 eq.). The mixture is stirred at 65° C. overnight. After aqueous work-up, the organic residue is purified by flash chromatography to yield the title compound as an orange solid.

¹H NMR (300 MHz, CDCl₃), δ (ppm): 8.06-8.02 (m, 1H), 7.78-7.66 (m, 3H), 5.09 (dd, 1H), 4.53 (dd, 1H), 4.07-3.98 (m, 1H), 3.65-3.55 (m, 1H), 3.58 (s, 3H), 3.45-3.36 (m, 1H), 3.18-3.10 (m, 1H), 1.77-1.61 (m, 5H), 1.41-1.24 (m, 1H), 0.96-0.85 (m, 12H)

MS⁺: 470 (M+H)

### Synthesis of I (S)-2-[(S-3-isobutyl-4-(o-nitrophenylsulfonyl)-2-oxo-1-piperazinyl]-4-methylvaleric acid

To a solution of methyl (S)-2-[(S)-3-isobutyl-4-(o-nitrophenylsulfonyl)-2-oxo-1-piperazinyl]-4-methylvalerate (1 eq.) in methanol is added a solution of lithium hydroxide (1.5 eq.) in water. The mixture is stirred at room temperature for 2 h then concentrated. The residue is acidified, extracted and concentrated to a gum that is re-crystallized to yield the title compound as a colourless solid.

¹H NMR (300 MHz, CDCl₃), δ (ppm): 8.85-7.85 (br s, 1H), 8.06-8.01 (m, 1H), 7.78-7.63 (m, 3H), 5.09 (dd, 1H), 4.59 (m, 1H), 4.14-4.05 (m, 1H), 3.65-3.54 (m, 1H), 3.45-3.33 (m, 1H), 3.17-3.09 (m, 1H), 1.78-1.61 (m, 5H), 1.45-1.30 (m, 1H), 0.97-0.87 (m, 12H)

MS⁻: 454 (M−H)

### Synthesis of (S)-1-[(S)-1-({4-[(1-cyclopropyl-1H-imidazol-2-yl)methyl]-1-piperidyl}carbonyl)-3-methylbutyl]-3-isobutyl-2-piperazinone following synthetic method A (Scheme 1)

### Synthesis of II

### Synthesis of tert-butyl 4-(2-hydroxyethyl)-1-piperidinecarboxylate

To a solution of 2-(4-piperidyl)ethanol (1 eq.) in a mixture THF/water is added di-tert-butyl dicarbonate (1.3 eq.) and sodium bicarbonate (2 eq.). The mixture is stirred at room temperature overnight. After aqueous work-up, the residue is purified by flash chromatography to yield the title compound as a light yellow oil.

¹H NMR (300 MHz, CDCl₃), δ (ppm): 4.08 (br d, 2H), 3.71 (t, 2H), 2.70 (br t, 2H), 1.73-1.47 (m, 6H), 1.46 (s, 9H), 1.21-1.04 (m, 2H)

### Synthesis of tert-butyl 4-(formylmethyl)-1-piperidinecarboxylate

To a solution of tert-butyl 4-(2-hydroxyethyl)-1-piperidinecarboxylate (1 eq.) in DCM is added DMP (2 eq.). The mixture is stirred at room temperature overnight. After aqueous work-up, the residue is purified by flash chromatography to yield the title compound as a yellow oil.

¹H NMR (300 MHz, CDCl₃), δ (ppm): 9.78 (br s, 1H), 4.08 (br d, 2H), 2.74 (br t, 2H), 2.39 (d, 2H), 2.12-1.89 (m, 1H), 1.79-1.64 (m, 2H), 1.45 (s, 9H), 1.26-1.10 (m, 2H)

### Synthesis of tert-butyl 4-[(1-cyclopropyl-1H-imidazol-2-yl)methyl]-1-piperidinecarboxylate

To a solution of tert-butyl 4-(formylmethyl)-1-piperidinecarboxylate (1 eq.) and glyoxal (1.2 eq.) in methanol is added cyclopropylamine (2 eq.) and ammonium acetate (1 eq.). The mixture is stirred at room temperature overnight then concentrated. The residue is purified by flash chromatography to yield the title compound as a yellow oil.

¹H NMR (300 MHz, CDCl₃), δ (ppm): 6.89 (s, 1H), 6.75 (s, 1H), 4.12-3.93 (m, 2H), 3.16-3.07 (m, 1H), 2.77-2.58 (m, 4H), 1.72-1.57 (m, 2H), 1.40 (s, 9H), 1.22-0.86 (m, 7H)

### Synthesis of II, 1-cyclopropyl-2-[(4-piperidyl)methyl]-1H-imidazole

To a cooled solution of tert-butyl 4-[(1-cyclopropyl-1H-imidazol-2-yl)methyl]-1-piperidinecarboxylate in DCM is added TFA. The solution is stirred at room temperature for 1 h then concentrated and the residue is neutralized with sodium hydroxide to yield the title compound as a yellow gum.

¹H NMR (300 MHz, CD₃OD), δ (ppm): 7.06 (s, 1H), 6.91 (s, 1H), 3.27-3.20 (m, 2H), 2.95-2.79 (m, 4H), 2.20-2.05 (m, 1H), 1.86-1.73 (m, 2H), 1.56-1.25 (m, 3H), 1.08-0.99 (m, 2H), 0.94-0.86 (m, 2H)

### Synthesis of (S)-1-[(S)-1-({4-[(1-cyclopropyl-1H-imidazol-2-yl)methyl]-1-piperidyl}carbonyl)-3-methylbutyl]-3-isobutyl-4-(o-nitrophenylsulfonyl)-2-piperazinone

To a cooled solution of I (vide supra) (1 eq.) and II (1-cyclopropyl-2-[(4-piperidyl)methyl]-1H-imidazole) (1 eq.) in DMF is added HATU reagent (2 eq.). The solution is stirred at room temperature overnight. After an aqueous work-up, the organic residue is purified by flash chromatography to yield the title compound as a yellow oil.

¹H NMR (300 MHz, CDCl₃), δ (ppm): 8.12-8.04 (m, 1H), 7.84-7.64 (m, 3H), 7.07-6.90 (m, 2H), 5.62 (t, 0.55H), 5.48 (t, 0.45H), 4.54-4.33 (m, 2H), 4.24-4.14 (m, 0.5H), 4.03-3.84 (m, 1.5H), 3.57-3.43 (m, 1H), 3.40-3.00 (m, 4H), 2.86-2.78 (m, 1.5H), 2.71-2.33 (m, 1.5H), 2.27-2.04 (m, 1H), 1.79-1.30 (m, 9H), 1.21-1.10 (m, 2H), 1.05-0.95 (m, 2H), 0.95-0.83 (m, 12H), 0.83-0.76 (m, 2H)

MS⁺: 643 (M+H)

### Synthesis of (S)-1-[(S)-1-({4-[(1-cyclopropyl-1H-imidazol-2-yl)methyl]-1-piperidyl}carbonyl)-3-methylbutyl]-3-isobutyl-2-piperazinone

A mixture of (S)-1-[(S)-1-({4-[(1-cyclopropyl-1H-imidazol-2-yl)methyl]-1-piperidyl}carbonyl)-3-methylbutyl]-3-isobutyl-4-(o-nitrophenylsulfonyl)-2-piperazinone (1 eq.), 2-mercaptoethanol (2 eq.) and cesium carbonate (3 eq.) in DMF is stirred at room temperature for 2 h. After filtration, concentration and flash chromatography purification, the title compound is obtained as a colourless oil.

¹H NMR (300 MHz, CD₃OD), δ (ppm): 6.98 (s, 1H), 6.82 (s, 1H), 5.63-5.52 (m, 1H), 4.58-4.42 (m, 1H), 4.19-3.99 (m, 1H), 3.48-3.40 (m, 1H), 3.38-3.27 (m, 2H), 3.19-3.03 (m, 2H), 2.98-2.85 (m, 1H), 2.80 (d, 2H), 2.77-2.61 (m, 1H), 2.27-2.09 (m, 1H), 1.90-1.43 (m, 8H), 1.30-1.06 (m, 4H), 1.04-0.86 (m, 15H)

MS⁺: 458 (M+H), 480 (M+Na)

### Synthesis of (S)-1-[(S)-3-methyl-1-({4-[2-(4-methyl-1-piperazinyl)-2-oxoethyl]-1-piperidyl}carbonyl)butyl]-3-isobutyl-2-piperazinone following synthetic method B (Scheme 2)

### Synthesis of III (methyl (1-{(S)-2-[(S)-3-isobutyl-4-(o-nitrophenylsulfonyl)-2-oxo-1-piperazinyl]-4-methylvaleryl}-4-piperidyl)acetate) by method B1

HATU reagent (1.5 eq.) is added to a cooled solution of IIIa ((S)-2-[(S)-3-isobutyl-4-(o-nitrophenylsulfonyl)-2-oxo-1-piperazinyl]-4-methylvaleric acid, vide supra) (1 eq.) and IIIb (methyl (4-piperidyl)acetate) (2 eq.) in DMF. The solution is stirred at room temperature overnight. After an aqueous work-up, the organic residue is purified by flash chromatography to yield the title compound as a light yellow solid.

¹H NMR (300 MHz, CDCl₃), δ (ppm): 8.13-8.05 (m, 1H), 7.81-7.67 (m, 3H), 5.65-5.57 (m, 0.5H), 5.54-5.46 (m, 0.5H), 4.52-4.39 (m, 2H), 4.21-4.12 (m, 0.5H), 4.00-3.86 (m, 1.5H), 3.69 (s, 3H), 3.57-3.32 (m, 1.5H), 3.30-3.16 (m, 1.5H), 3.01 (t, 0.5H), 2.77 (t, 0.5H), 2.62-2.40 (m, 1H), 2.32-2.18 (m, 2H), 2.08-1.91 (m, 1H), 1.82-1.32 (m, 8H), 1.22-0.97 (m, 2H), 0.97-0.78 (m, 12H)

### Synthesis of (1-{(S)-2-[(S)-3-isobutyl-4-(o-nitrophenylsulfonyl)-2-oxo-1-piperazinyl]-4-methylvaleryl}-4-piperidyl)acetic acid (part of method B2)

Sodium hydroxide (1.5 eq.) is added to a solution of methyl (1-{(S)-2-[(S)-3-isobutyl-4-(o-nitrophenylsulfonyl)-2-oxo-1-piperazinyl]-4-methylvaleryl}-4-piperidyl)acetate (1 eq.) in a mixture methanol/water. The solution is stirred at room temperature overnight then acidified and extracted to yield the titled compound as a light yellow solid.

¹H NMR (300 MHz, CDCl₃), δ (ppm): 8.14-8.07 (m, 1H), 7.81-7.67 (m, 3H), 5.67-5.60 (m, 0.5H), 5.56-5.48 (m, 0.5H), 4.55-4.40 (m, 2H), 4.24-4.14 (m, 0.5H), 4.00-3.88 (m, 1.5H), 3.58-3.34 (m, 1.5H), 3.31-3.16 (m, 1.5H), 3.07-2.96 (m, 0.5H), 2.81-2.69 (m, 0.5H), 2.62-2.41 (m, 1H), 2.36-2.22 (m, 2H), 2.09-1.91 (m, 1H), 1.86-1.32 (m, 8H), 1.25-0.99 (m, 2H), 0.97-0.77 (m, 12H)

MS⁻: 579 (M−H)

### Synthesis of (S)-1-[(S)-3-methyl-1-({4-[2-(4-methyl-1-piperazinyl)-2-oxoethyl]-1-piperidyl}carbonyl)butyl]-3-isobutyl-4-(o-nitrophenylsulfonyl)-2-piperazinone (part of method B2)

Oxalyl chloride (2 eq.) is added to a cooled solution of (1-{(S)-2-[(S)-3-isobutyl-4-(o-nitrophenylsulfonyl)-2-oxo-1-piperazinyl]-4-methylvaleryl}-4-piperidyl)acetic acid (1 eq.) and catalytic DMF in DCM. The solution is stirred at room temperature for 3 hours then 1-methylpiperazine (5 eq.) is added at 0° C. and the solution is stirred at room temperature overnight. Concentration and flash chromatography purification yield to the title compound as a light yellow gum.

¹H NMR (300 MHz, CDCl₃), δ (ppm): 8.10-8.04 (m, 1H), 7.82-7.67 (m, 3H), 5.67-5.60 (m, 0.6H), 5.53-5.46 (m, 0.4H), 4.55-4.39 (m, 2H), 4.20-4.11 (m, 0.6H), 3.97-3.85 (m, 1.4H), 3.70-3.60 (m, 2H), 3.56-3.32 (m, 3.6H), 3.28-3.15 (m, 1.4H), 3.02 (t, 0.6H), 2.74-2.62 (m, 0.4H), 2.62-2.43 (m, 1H), 2.43-2.34 (m, 4H), 2.34-1.96 (m, 6H), 1.87-1.33 (m, 8H), 1.25-0.97 (m, 2H), 0.96-0.80 (m, 12H)

### Synthesis of (S)-1-[(S)-3-methyl-1-({4-[2-(4-methyl-1-piperazinyl)-2-oxoethyl]-1-piperidyl}carbonyl)butyl]-3-isobutyl-2-piperazinone (part of method B2)

A mixture of (S)-1-[(S)-3-methyl-1-({4-[2-(4-methyl-1-piperazinyl)-2-oxoethyl]-1-piperidyl}carbonyl)butyl]-3-isobutyl-4-(o-nitrophenylsulfonyl)-2-piperazinone (1 eq.), polymer-supported benzyl mercaptan (2.5-5 eq.) and cesium carbonate (3 eq.) in DMF is shaken at room temperature overnight. After filtration, concentration, and flash chromatography purification if necessary, the title compound is obtained as a light yellow gum.

¹H NMR (300 MHz, CD₃OD), δ (ppm): 5.62-5.52 (m, 1H), 4.56-4.42 (m, 1H), 4.18-4.01 (m, 1H), 3.67-3.56 (m, 4H), 3.47-3.41 (m, 1H), 3.39-3.29 (m, 2H), 3.19-3.04 (m, 2H), 3.00-2.87 (m, 1H), 2.79-2.63 (m, 1H), 2.50-2.40 (m, 4H), 2.40-2.35 (m, 2H), 2.33 (s, 3H), 2.14-2.00 (m, 1H), 1.90-1.46 (m, 8H), 1.27-1.04 (m, 2H), 1.02-0.92 (m, 12H)

MS⁺: 478 (M+H), 500 (M+Na).

### Examples 1-319

The following compounds have been synthesized according to the methods outlined supra and the compounds have been characterized by their nmr signals. Syntheses of precursor I was according to Method C. Starting material for precursor I used was as indicated in Table 1. Syntheses of precursor II and III was as indicated in Tables 2 and 3 below.

TABLE 1
Starting material for precursor I

| Ex./Cmpd. # | Precursor Ia | Precursor Ib | Precursor Ic |
|---|---|---|---|
| 1-7, 9-32, | N-(2-nosyl)-L-leucine | L-leucine methyl ester | 1,2-dibromoethane |
| 36-47, 52-60, | | hydrochloride | |
| 63-87, 95-103, | | | |
| 111, 124, 125, | | | |
| 135-138, | | | |
| 141-229 | | | |
| 8, 33, 34, 109 | (S)-3-cyclopropyl-2-(o- | Methyl (S)-2-amino-3- | 1,2-dibromoethane |
| | nitrophenylsulfonylamino)propionic | cyclopropylpropionate hydrochloride | |
| | acid | | |
| 35, 115, 133, | N-(2-nosyl)-L-α-neopentylglycine | Methyl (S)-2-amino-3- | 1,2-dibromoethane |
| 134 | | cyclopropylpropionate hydrochloride | |
| 48, 51, 88 | N-(2-nosyl)-L-leucine | L-phenylalanine methyl | 1,2-dibromoethane |
| | | ester hydrochloride | |
| 49, 50, 90 | N-(2-nosyl)-L-leucine | L-valine methyl ester | 1,2-dibromoethane |
| | | hydrochloride | |
| 61, 62, 91 | (S)-3-cyclopropyl-2-(o- | L-leucine methyl ester | 1,2-dibromoethane |
| | nitrophenylsulfonylamino)propionic | hydrochloride | |
| | acid | | |
| 89 | N-(2-nosyl)-L-phenylalanine | L-leucine methyl ester | 1,2-dibromoethane |
| | | hydrochloride | |
| 92 | (S)-3-cyclopropyl-2-(o- | Methyl (S)-2-aminovalerate | 1,2-dibromoethane |
| | nitrophenylsulfonylamino)propionic | hydrochloride | |
| | acid | | |
| 93 | (S)-3-cyclopropyl-2-(o- | Methyl (S)-2-aminohexanoate | 1,2-dibromoethane |
| | nitrophenylsulfonylamino)propionic | hydrochloride | |
| | acid | | |
| 94 | (S)-3-cyclopropyl-2-(o- | Methyl (S)-2-amino-4,4- | 1,2-dibromoethane |
| | nitrophenylsulfonylamino)propionic | dimethylvalerate hydrochloride | |
| | acid | | |
| 104 | N-(2-nosyl)-L-leucine | Methyl (S)-2-aminohexanoate | 1,2-dibromoethane |
| | | hydrochloride | |
| 105 | N-(2-nosyl)-L-leucine | Methyl (S)-2-amino-3- | 1,2-dibromoethane |
| | | cyclopropylpropionate hydrochloride | |
| 106 | N-(2-nosyl)-L-norleucine | Methyl (S)-2-amino-3- | 1,2-dibromoethane |
| | | cyclopropylpropionate hydrochloride | |
| 107 | N-(2-nosyl)-L-norleucine | Methyl (S)-2-aminohexanoate | 1,2-dibromoethane |
| | | hydrochloride | |
| 108 | N-(2-nosyl)-L-norleucine | Methyl (S)-2-aminovalerate | 1,2-dibromoethane |
| | | hydrochloride | |
| 110 | N-(2-nosyl)-L-norleucine | L-leucine methyl ester | 1,2-dibromoethane |
| | | hydrochloride | |
| 112 | N-(2-nosyl)-L-α-neopentylglycine | Methyl (S)-2-aminovalerate | 1,2-dibromoethane |
| | | hydrochloride | |
| 113 | N-(2-nosyl)-L-α-neopentylglycine | Methyl (S)-2-aminohexanoate | 1,2-dibromoethane |
| | | hydrochloride | |
| 114 | N-(2-nosyl)-L-α-neopentylglycine | L-leucine methyl ester | 1,2-dibromoethane |
| | | hydrochloride | |
| 116 | N-(2-nosyl)-L-leucine | Methyl (S)-2-amino-4,4- | 1,2-dibromoethane |
| | | dimethylvalerate hydrochloride | |
| 117 | N-(2-nosyl)-L-norvaline | Methyl (S)-2-amino-4,4- | 1,2-dibromoethane |
| | | dimethylvalerate hydrochloride | |
| 118 | N-(2-nosyl)-L-α-neopentylglycine | Methyl (S)-2-amino-4,4- | 1,2-dibromoethane |
| | | dimethylvalerate hydrochloride | |
| 119, 139 | N-(2-nosyl)-L-leucine | L-alanine methyl ester | 1,2-dibromoethane |
| | | hydrochloride | |
| 120 | N-(2-nosyl)-L-leucine | Methyl (S)-2-amino-3- | 1,2-dibromoethane |
| | | cyclohexylpropionate hydrochloride | |
| 121, 140 | N-(2-nosyl)-L-alanine | L-leucine methyl ester | 1,2-dibromoethane |
| | | hydrochloride | |
| 122 | (S)-3-cyclohexyl-2-(o- | L-leucine methyl ester | 1,2-dibromoethane |
| | nitrophenylsulfonylamino) propionic | hydrochloride | |
| | acid | | |
| 123 | N-(2-nosyl)-L-norleucine | Methyl (S)-2-amino-4,4- | 1,2-dibromoethane |
| | | dimethylvalerate hydrochloride | |
| 126 | N-(2-nosyl)-L-leucine | L-isoleucine methyl | 1,2-dibromoethane |
| | | ester hydrochloride | |
| 127 | N-(2-nosyl)-L-isoleucine | L-leucine methyl ester | 1,2-dibromoethane |
| | | hydrochloride | |
| 128 | N-(2-nosyl)-L-norvaline | L-leucine methyl ester | 1,2-dibromoethane |
| | | hydrochloride | |
| 129 | N-(2-nosyl)-L-norvaline | Methyl (S)-2-aminohexanoate | 1,2-dibromoethane |
| | | hydrochloride | |
| 130 | N-(2-nosyl)-L-norvaline | Methyl (S)-2-aminovalerate | 1,2-dibromoethane |
| | | hydrochloride | |
| 131 | N-(2-nosyl)-L-leucine | Methyl (S)-2-aminovalerate | 1,2-dibromoethane |
| | | hydrochloride | |
| 132 | N-(2-nosyl)-L-norvaline | Methyl (S)-2-amino-3- | 1,2-dibromoethane |
| | | cyclopropylpropionate hydrochloride | |

All starting materials for precursor I have been obtained from Sigma-Aldrich, except (S)-2-Amino-3-cyclohexylpropionic acid, (S)-2-Amino-3-cyclopropylpropionic acid, Methyl (S)-2-aminovalerate hydrochloride, Methyl (S)-2-aminohexanoate hydrochloride and Methyl (S)-2-amino-3-cyclohexylpropionate hydrochloride, (all Combi-Blocks), Methyl (S)-2-amino-4,4-dimethylvalerate hydrochloride (Enamine BB), and Methyl (S)-2-amino-3-cyclopropylpropionate hydrochloride (Activate Scientific).

TABLE 2
Compounds made through Method A

| | | | | Characteristic | |
|---|---|---|---|---|---|
| | Ex. | | | ¹H NMR | |
| | Cmpd | | Precursor II | signals (300 | |
| Structure | # | name | synthesis | MHz) | Activity |
| 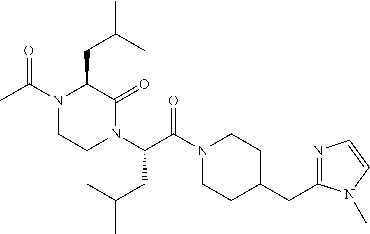 [SMILES: CC(=O)N1CCN([C@@H](CC(C)C)C(=O)N2CCC(Cc3nccn3C)CC2)C(=O)[C@@H]1CC(C)C] | 1 | (S)-1-[(S)-3-Methyl- 1-({4-[(1-methyl- 1H-imidazol-2- yl)methyl]-1- piperidyl}car- bonyl)butyl]-4- acetyl-3- isobutyl-2- piperazinone | 1-methyl-2-[(4- piperidyl)methyl]- 1H-imidazole (Enamine BB) | CD₃OD: δ 6.98, (s, 1H), 3.64 (br s, 3H), 2.17 (m, 3H), 0.98 (m, 12H) | +++ |
| 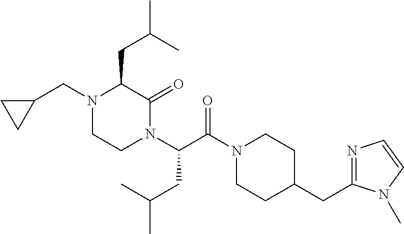 [SMILES: CC(C)C[C@H]1C(=O)N([C@@H](CC(C)C)C(=O)N2CCC(Cc3nccn3C)CC2)CCN1CC1CC1] | 2 | (S)-1-[(S)-3-Methyl- 1-({4-[(1-methyl- 1H-imidazol-2- yl)methyl]-1- piperidyl}car- bonyl)butyl]-4- (cyclopropylmethyl)- 3-isobutyl-2- piperazinone | 1-methyl-2-[(4- piperidyl)methyl]- 1H-imdiazole- (Enamine BB) | CD₃OD: δ 6.97 (s, 1H), 3.64 (br s, 3H), 0.95 (m, 14H), 0.15 (m, 2H) | +++ |
| 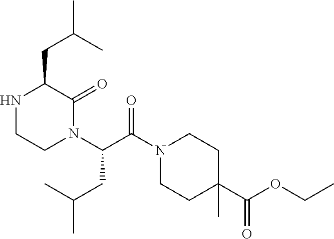 [SMILES: CCOC(=O)C1(C)CCN(C(=O)[C@H](CC(C)C)N2CCN[C@@H](CC(C)C)C2=O)CC1] | 3 | Ethyl 1-{(S)-2-[(S)- 3-isobutyl-2-oxo-1- piperazinyl]-4- methylvaleryl}-4- methyl-4- piperidinecarboxylate | Ethyl 4-methyl- 4- piperidinecar- boxylate (Combi- Blocks) | CD₃OD: δ 5.47 (m, 1H), 2.11- 1.93 (br m, 2H), 1.22-1.12 (m, 6H), 0.88 (m, 12H) | ++ |
| 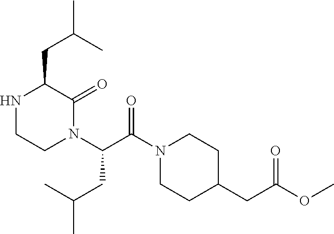 [SMILES: COC(=O)CC1CCN(C(=O)[C@H](CC(C)C)N2CCN[C@@H](CC(C)C)C2=O)CC1] | 4 | Methyl (1-{(S)-2- [(S)-3-isobutyl-2- oxo-1-piperazinyl]- 4-methylvaleryl}-4- piperidyl)acetate | Methyl (4- piperidyl)acetate (Combi-Blocks) | CD₃OD: δ 5.55 (m, 1H), 4.47 (t, 1H), 3.66 (s, 3H), 2.30 (d, 2H), 0.95 (m, 12H) | ++ |
| 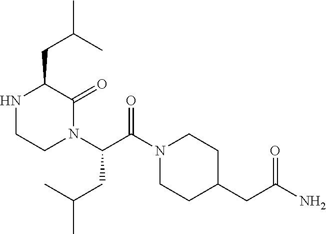 [SMILES: CC(C)C[C@@H]1NCCN([C@@H](CC(C)C)C(=O)N2CCC(CC(N)=O)CC2)C1=O] | 5 | (1-{(S)-2-[(S)-3- Isobutyl-2-oxo-1- piperazinyl]-4- methylvaleryl}-4- piperidyl)actamide | (4-Piperidyl)acet- amide (Enamine BB) | CD₃OD: δ 5.56 (m, 1H), 4.47 (t, 1H), 2.15 (m, 2H), 2.03 (m, 1H), 0.95 (m, 12H) | +* |
| 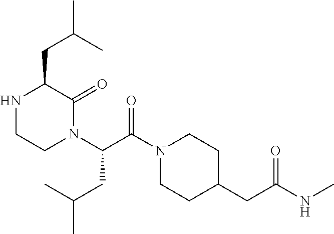 [SMILES: CNC(=O)CC1CCN(C(=O)[C@H](CC(C)C)N2CCN[C@@H](CC(C)C)C2=O)CC1] | 6 | (S)-1-[(S)-3-Methyl- 1-({4-[2- (methylamino)-2- oxoethyl]-1- piperidyl}car- bonyl)butyl]-3- isobutyl-2- piperazinone | 1- (Methylamino)- 2-(4-piperidyl)-1- ethanone (Enamine BB) | CD₃OD: δ 5.55 (m, 1H), 4.46 (t, 1H), 2.71 (s, 3H), 1.24-1.03 (br m, 3H), 0.95 (m, 12H) | +* |
| 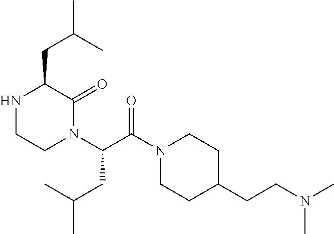 [SMILES: CC(C)C[C@@H]1NCCN([C@@H](CC(C)C)C(=O)N2CCC(CCN(C)C)CC2)C1=O] | 7 | (S)-1-[(S)-1-({4-[2- (Dimethylamino)eth- yl]-1- piperidyl}car- bonyl)-3- methylbutyl]-3- isobutyl-2- piperazinone | tert-Butyl 4-[2- hydroxyethyl)-1- piperidinecar- boxylate; DMP oxidation to aldehyde, reductive amination with dimethylamine, BOC removal | CD₃OD: δ 5.55 (m, 1H), 4.46 (t, 1H), 2.43 (t, 2H), 2.29 (s, 6H), 0.95 (m, 12H) | ++++ |
| 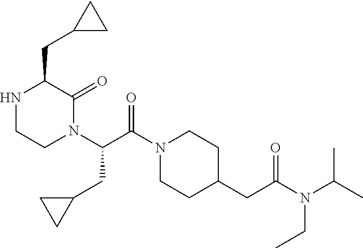 [SMILES: CCN(C(=O)CC1CCN(C(=O)[C@H](CC2CC2)N2CCN[C@@H](CC3CC3)C2=O)CC1)C(C)C] | 8 | (S)-1-[(S)-2-(4-{2- [N- Ethyl(isopropyl)ami- no]-2-oxoethyl}-1- piperidyl)-1- (cyclopropylmethyl)- 2-oxoethyl]-3- (cyclopropylmethyl)- 2-piperazinone | (1-tert- Butoxycarbonyl- 4- piperidyl)acetic acid: amide coupling with N- Ethyl(isopropyl) amine, BOC removal | CD₃OD: δ 5.55 (m, 1H), 1.27- 1.08 (br m, 12H), 0.47 (m, 4H), 0.13 (m, 4H) | +* |
| 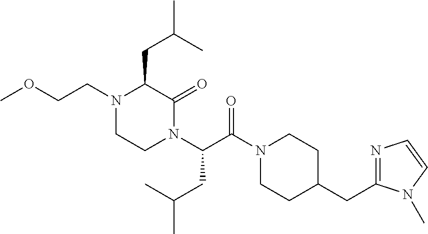 [SMILES: COCCN1CCN([C@@H](CC(C)C)C(=O)N2CCC(Cc3nccn3C)CC2)C(=O)[C@@H]1CC(C)C] | 9 | (S)-1-[(S)-3-Methyl- 1-({4-[(1-methyl- 1H-imidazol-2- yl)methyl]-1- piperidyl}car- bonyl)butyl]-3- isobutyl-4- (2-methoxyethyl)- 2-piperazinone | 1-methyl-2-[(4- piperidyl)methyl]- 1H-imidazole (Enamine BB) | CD₃OD: δ 6.98 (s, 1H), 6.86 (s, 1H), 5.55 (m, 1H), 3.64, (s, 3H), 3.32 (m, 3H), 0.95 (m, 14H) | +++ |
| 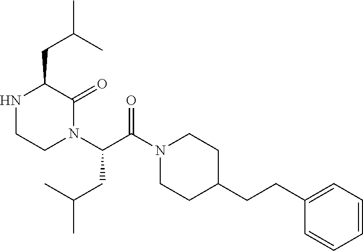 [SMILES: CC(C)C[C@@H]1NCCN([C@@H](CC(C)C)C(=O)N2CCC(CCc3ccccc3)CC2)C1=O] | 10 | (S)-1-{(S)-3-Methyl- 1-[(4-phenethyl-1- piperidyl)car- bonyl]butyl}-3- isobutyl-2- piperazinone | 4- phenethylpiperidine (Enamine BB) | CD₃OD: δ 7.29-7.11 (br m, 5H), 5.57 (m, 1H), 1.81 (m, 4H), 1.57 (m, 7H), 0.96 (m, 13H) | +++ |
| 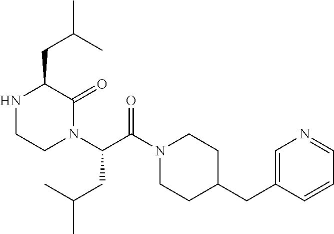 [SMILES: CC(C)C[C@@H]1NCCN([C@@H](CC(C)C)C(=O)N2CCC(Cc3cccnc3)CC2)C1=O] | 11 | (S)-1-[(S)-3-Methyl- 1-({4-[(3- pyridyl)methyl]-1- piperidyl}car- bonyl)butyl]-3- isobutyl-2- piperazinone | 3-(piperidin-4- ylmethyl)pyridine (Enamine BB) | CD₃OD: δ 8.39 (m, 2H), 7.41 (m, 1H), 7.18 (m, 1H), 5.53 (m, 1H), 4.51 (t, 1H), 0.89 (m, 14H) | +++ |
| 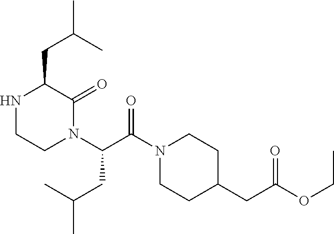 [SMILES: CCOC(=O)CC1CCN(C(=O)[C@H](CC(C)C)N2CCN[C@@H](CC(C)C)C2=O)CC1] | 12 | Ethyl (1-{(S)-2-[(S)- 3-isobutyl-2-oxo-1- piperazinyl]-4- methylvaleryl}-4- piperidyl)acetate | Ethyl (4- piperidyl)acetate (Combi-Blocks) | CD₃OD: δ 5.64 (m, 1H), 4.56 (t, 1H), 4.21 (q, 2H), 2.37 (m, 2H), 1.33 (t, 3H), 1.04 (m, 12H) | +++ |
| 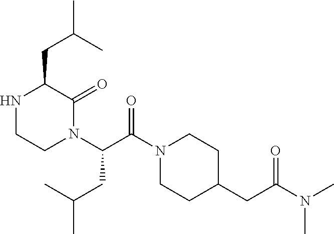 [SMILES: CC(C)C[C@@H]1NCCN([C@@H](CC(C)C)C(=O)N2CCC(CC(=O)N(C)C)CC2)C1=O] | 13 | (S)-1-[(S)-1-({4-[2- (Dimethylamino)- 2-oxoethyl]-1- piperidyl}carbonyl)- 3-methylbutyl]-3- isobutyl-2- piperazinone | 1- (Dimethylamino)- 2-(4-piperidyl)- 1-ethanone (Enamine BB) | CD₃OD: δ 5.61 (m, 1H), 4.53 (t, 1H), 3.12 (s, 3H), 2.99 (s, 3H), 2.40 (m, 3H), 1.01 (m, 12H) | +* |
| 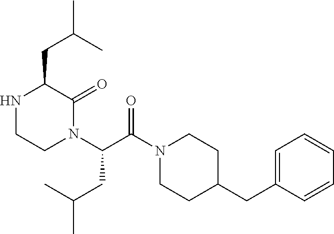 [SMILES: CC(C)C[C@@H]1NCCN([C@@H](CC(C)C)C(=O)N2CCC(Cc3ccccc3)CC2)C1=O] | 14 | (S)-1-{(S)-1-[(4- Benzyl-1- piperidyl)carbonyl]- 3-methylbutyl}-3- isobutyl-2- piperazinone | 4-Benzyl- piperidine | CD₃OD: δ 7.18 (m, 5H), 5.57 (m, 1H), 4.52 (t, 1H), 1.67 (m, 10H), 0.93 (m, 12H) | +++ |
| 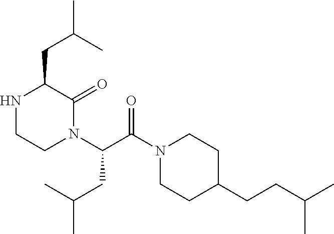 [SMILES: CC(C)CCC1CCN(C(=O)[C@H](CC(C)C)N2CCN[C@@H](CC(C)C)C2=O)CC1] | 15 | (S)-1-{(S)-1-[(4- Isopentyl-1- piperidyl)carbonyl]- 3-methylbutyl}-3- isobutyl-2- piperazinone | 4-Isopentyl- piperidine (Enamine BB) | CD₃OD: δ 5.54 (m, 1H), 4.48 (t, 1H), 1.58 (m, 12H), 1.14 (m, 4H), 0.93 (m, 12H), 0.81 (m, 6H) | +++ |
| 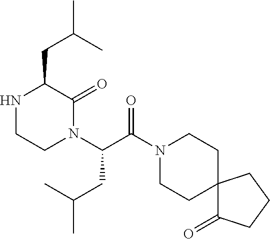 [SMILES: CC(C)C[C@@H]1NCCN([C@@H](CC(C)C)C(=O)N2CCC3(CCCC3=O)CC2)C1=O] | 16 | (S)-1-[(S)-1-{(8- Azaspiro[4.5]decan- 8-oyl)carbonyl}-3- methylbutyl]-3- isobutyl-2- piperazinone | 8-azaspiro[4.5]dec- an-1-one (Enamine BB) | CDCl3: δ, 5.56 (q, 1H), 4.45 (t, 1H), 2.35 (m 2H), 0.95, (m, 12H). | ++ |
| 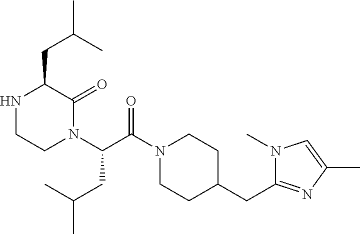 [SMILES: Cc1cn(C)c(CC2CCN(C(=O)[C@H](CC(C)C)N3CCN[C@@H](CC(C)C)C3=O)CC2)n1] | 17 | (S)-1-[(S)-3-Methyl- 1-({4-[(1-methyl-4- methyl-1H- imidazol-2- yl)methyl]-1- piperidyl}car- bonyl)butyl]-3- isobutyl-2- piperazinone | 2-(4- piperidyl)ethanol; tBOC protection, oxidation to aldehyde, N- methyl imidazole construcution (Amide formation, oxidation, cyclization), tBOC removal | CD₃OD: δ 6.65 (s, 1H), 5.56 (q, 1H), 4.57 (t, 1H), 3.51 (s, 3H), 2.15 (s, 3H), 0.95 (m, 12H). | ++ |
| 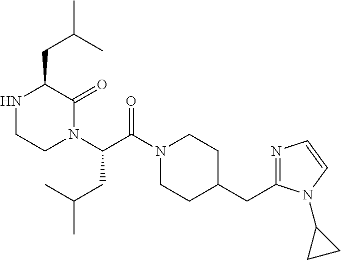 [SMILES: CC(C)C[C@@H]1NCCN([C@@H](CC(C)C)C(=O)N2CCC(Cc3nccn3C3CC3)CC2)C1=O] | 18 | (S)-1-[(S)-1-({4-[(1- Cyclopropyl-1H- imidazol-2- yl)methyl]-1- piperidyl}carbonyl)- 3-methylbutyl]-3- isobutyl-2- piperazinone | 2-(4- piperidyl)ethanol; tBOC protection, oxidation to aldehyde, N- cyclopropylamine imidazole construction (with ammonia, cyclopropylamine and glyoxal), tBOC removal | CD₃OD: δ, 6.95 (S, 1H), 6.82 (s, 1H), 5.50 (q, 1H), 4.45 (t, 1H), 0.95, (m, 16H). | ++ |
| 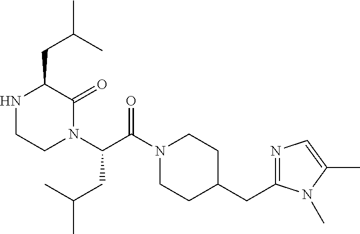 [SMILES: Cc1cnc(CC2CCN(C(=O)[C@H](CC(C)C)N3CCN[C@@H](CC(C)C)C3=O)CC2)n1C] | 19 | (S)-1-[(S)-3-Methyl- 1-({4-[(1-methyl-5- methyl-1H- imidazol-2- yl)methyl]-1- piperidyl}car- bonyl)butyl]-3- isobutyl-2- piperazinone | 2-(4- piperidyl)ethanol; tBOC protection, oxidation to aldehyde, N- methyl imidazole construction (Amide formation, oxidation, cyclization, tBOC removal | CD₃OD: δ, 6.75 (s, 1H), 5.55 (q, 1H), 4.45 (t, 1H), 3.55 (s, 3H), 2.50 (s, 3H), 0.96, (m, 12H). | ++ |
| 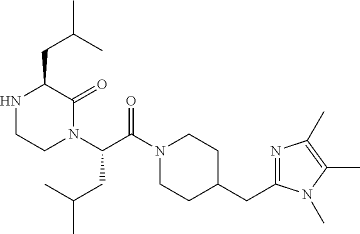 [SMILES: Cc1nc(CC2CCN(C(=O)[C@H](CC(C)C)N3CCN[C@@H](CC(C)C)C3=O)CC2)n(C)c1C] | 20 | (S)-1-[(S)-3-Methyl- 1-({4-[(1-methyl- 4,5-dimethyl-1H- imdiazol-2- yl)methyl]-1- piperidyl}car- bonyl)butyl]-3- isobutyl-2- piperazinone | 2-(4- piperidyl)ethanol; tBOC protection, DMP oxidation to aldehyde, imidazole formation with biacetyl, NH₄OAc and methylamine, BOC removal. | ¹H NMR (CD₃OD) δ 0.89 (m, 12H), 1.22 (m, 1H), 1.67 (m, 8H), 2.67 (m. 3H), 3.01 (m, 4H), 3.42 (m, 2H), 3.54 (s, 3H), 5.55 (m, 1H). | +++ |
| 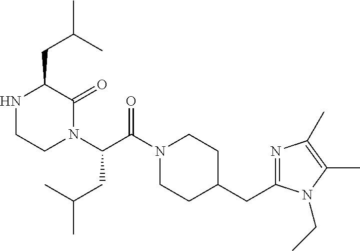 [SMILES: CCn1c(CC2CCN(C(=O)[C@H](CC(C)C)N3CCN[C@@H](CC(C)C)C3=O)CC2)nc(C)c1C] | 21 | (S)-1-[(S)-1-({4-[(1- Ethyl-4,5-dimethyl- 1H-imidazol-2- yl)methyl]-1- piperidyl}carbonyl)- 3-methylbutyl]-3- isobutyl-2- piperazinone | 2-(4- piperidyl)ethanol; tBOC protection, DMP oxidation to aldehyde, imidazole formation with biacetyl, NH₄OAc and ethylamine, BOC removal. | ¹H NMR (CD₃OD) δ 0.96 (m, 12H), 1.17 (m, 2H), 1.28 (m, 3H), 1.54 (m, 3H), 1.74 (m, 6H), 2.04 (m, 1H), 2.67 (m, 3H), 3.96 (m, 2H) | +++ |
| 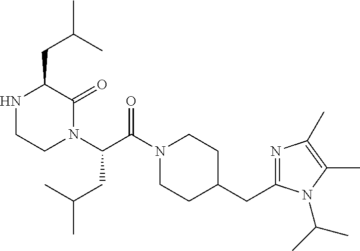 [SMILES: Cc1nc(CC2CCN(C(=O)[C@H](CC(C)C)N3CCN[C@@H](CC(C)C)C3=O)CC2)n(C(C)C)c1C] | 22 | (S)-1-[(S)-1-({4-[(1- Isopropyl-4,5- dimethyl-1H- imidazol-2- yl)methyl]-1- piperidyl}carbonyl)- 3-methylbutyl]-3- isobutyl-2- piperazinone | 2-(4- piperidyl)ethanol; tBOC protection, DMP oxidation to aldehyde, imidazole formation with biacetyl, NH₄OAc and isopropylamine, BOC removal. | ¹H NMR (CD₃OD) δ 0.96 (m, 12H), 1.22 (m, 2H), 1.56 (m, 9H), 2.66 (m, 1H), 2.85 (m, 2H), 5.56 (m, 1H) | ++ |
| 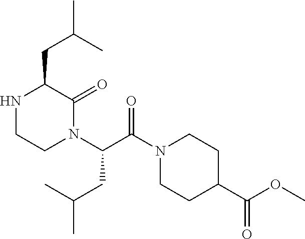 [SMILES: COC(=O)C1CCN(C(=O)[C@H](CC(C)C)N2CCN[C@@H](CC(C)C)C2=O)CC1] | 23 | Methyl 1-{(S)-2- [(S)-3-isobutyl-2- oxo-1-piperazinyl]- 4-methylvaleryl}-4- piperidinecar- boxylate | Methyl 4- piperidinecar- boxylate | CD₃OD: δ 5.49 (m, 1H), 3.61 (m, 3H), 1.93- 1.81 (br m, 2H), 0.88 (m, 12H) | +* |
| 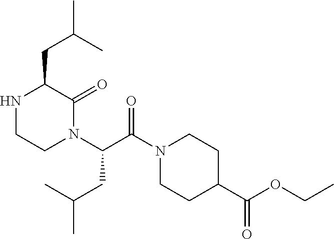 [SMILES: CCOC(=O)C1CCN(C(=O)[C@H](CC(C)C)N2CCN[C@@H](CC(C)C)C2=O)CC1] | 24 | Ethyl 1-{(S)-2-[(S)- 3-isobutyl-2-oxo-1- piperazinyl]-4- methylvaleryl}-4- piperidinecar- boxylate | Ethyl 4- piperidinecar- boxylate | CD₃OD: δ 5.52 (m, 1H), 1.96- 1.84 (br m, 2H), 1.21 (t, 3H), 0.91 (m, 12H) | +* |
| 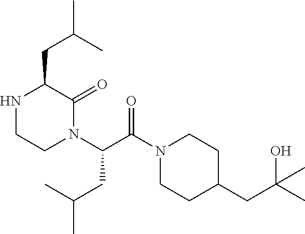 [SMILES: CC(C)C[C@@H]1NCCN([C@@H](CC(C)C)C(=O)N2CCC(CC(C)(C)O)CC2)C1=O] | 25 | (S)-1-[(S)-1-{[4-(2- Hydroxy-2- methylpropyl)-1- piperidyl]carbonyl}- 3-methylbutyl]-3- isobutyl-2- piperazinone | Methyl (4- piperidyl)acetate; benzyl protection, di- methylation, benzyl removal | CD₃OD: δ 5.57 (m, 1H), 4.43 (m, 1H), 1.23 (s, 6H), 0.97 (m, 12H) | ++ |
| 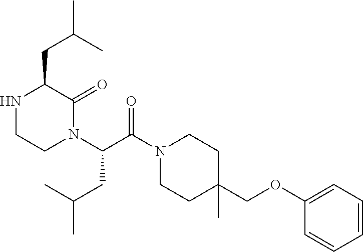 [SMILES: CC(C)C[C@@H]1NCCN([C@@H](CC(C)C)C(=O)N2CCC(C)(COc3ccccc3)CC2)C1=O] | 26 | (S)-1-[(S)-3-Methyl- 1-{[4-methyl-4- (phenoxymethyl)- 1- piperidyl]car- bonyl}butyl]-3- isobutyl-2- piperazinone | tert-Butyl 4- (hydroxymethyl)- 4-methyl-1- piperidinecar- yboxlate (Combi- Blocks); activation of alcohol, substitution with phenol, tBOC removal | CD₃OD: δ 7.26 (t, 2H), 5.60 (m, 1H), 1.17 (m, 3H), 0.97 (m, 12H) | +++ |
| 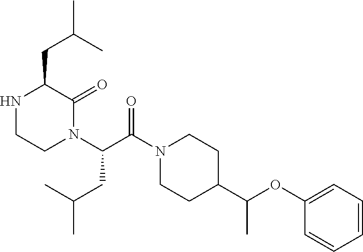 [SMILES: CC(C)C[C@@H]1NCCN([C@@H](CC(C)C)C(=O)N2CCC(C(C)Oc3ccccc3)CC2)C1=O] | 27 | (S)-1-[(S)-3-Methyl- 1-{[4-(1- phenoxyethyl)-1- piperidyl]car- bonyl}butyl]-3- isobutyl-2- piperazinone | tert-Butyl 4-(1- hydroxyethyl)-1- piperidinecar- boxylate (AstaTech); activation of alcohol, substitution with phenol, tBOC removal | CD₃OD: δ 7.26 (t, 2H), 5.59 (m, 1H), 1.26 (d, 3H), 0.96 (m, 12H) | +++ |
| 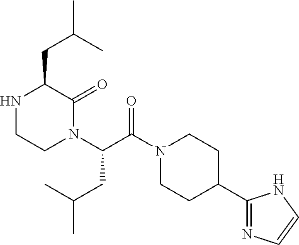 [SMILES: CC(C)C[C@@H]1NCCN([C@@H](CC(C)C)C(=O)N2CCC(c3ncc[nH]3)CC2)C1=O] | 28 | (S)-1-[(S)-1-{[4-(1H- Imidazol-2-yl)-1- piperidyl]carbonyl}- 3-methylbutyl]-3- isobutyl-2- piperazinone | 2-(4-Piperidyl)- 1H-imidazole (Enamine BB) | CD₃OD: δ 6.95 (s, 2H), 5.59 (m, 1H), 4.54 (m, 1H), 2.01 (d, 2H), 0.95 (m, 12H) | +* |
| 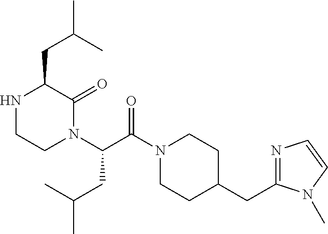 [SMILES: CC(C)C[C@@H]1NCCN([C@@H](CC(C)C)C(=O)N2CCC(Cc3nccn3C)CC2)C1=O] | 29 | (S)-1-[(S)-3-Methyl- 1-({4-[(1-methyl- 1H-imidazol-2- yl)methyl]-1- piperidyl}car- bonyl)butyl]-3- isobutyl-2- piperazinone | 1-methyl-2-[(4- piperidyl)methyl]- 1H-imidazole (Enamine BB) | CD₃OD: δ 6.98 (s, 1H), 6.86 (s, 1H), 5.55 (m, 1H), 4.47 (t, 1H), 4.07 (m, 1H), 3.64 (s, 3H), 3.42 (m, 1H), 3.33 (m, 1H), 3.19-2.98 (br m, 2H), 2.91 (m, 1H), 2.77-2.57 (br m, 3H), 2.04 (m, 1H), 1.92- 1.02 (br m, | +++ |
| | | | | 11H), 0.95 (m, | |
| | | | | 12H) | |
| 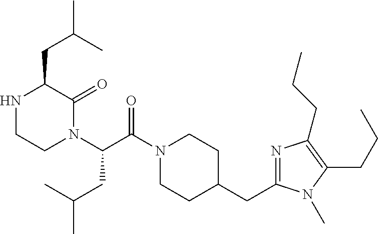 [SMILES: CCCc1nc(CC2CCN(C(=O)[C@H](CC(C)C)N3CCN[C@@H](CC(C)C)C3=O)CC2)n(C)c1CCC] | 30 | (S)-1-[(S)-3-Methyl- 1-({4-[(1-methyl- 4,5-dipropyl-1H- imidazol-2- yl)methyl]-1- piperidyl}car- bonyl)butyl]-3- isobutyl-2- piperazinone | p-toluene- sulfonylmethyl isocyanide; alkylation with 1- iodopropane in the presence of NaOH and NBu₄I, Van Leusen imidazole formation using butyraldehyde and methylamine, deprotonation with nBuLi | CD₃OD: δ 5.55 (m, 1H), 4.49 (t, 1H), 3.58 (s, 3H), 2.57 (t, 2H), 2.50 (t, 2H), 2.03 (m, 1H), 0.95 (m, 18H) | +++ |
| | | | followed by | | |
| | | | addition to tert- | | |
| | | | butyl 4-formyl-1- | | |
| | | | piperidinecarbox- | | |
| | | | ylate, mesylation | | |
| | | | of alcohol with | | |
| | | | methanesulfonyl | | |
| | | | chloride, | | |
| | | | catalytic | | |
| | | | hydrogenation | | |
| | | | using Pd/C and | | |
| | | | H₂ at 40 psi, BOC | | |
| | | | removal | | |
| 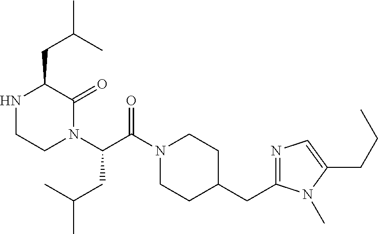 [SMILES: CCCc1cnc(CC2CCN(C(=O)[C@H](CC(C)C)N3CCN[C@@H](CC(C)C)C3=O)CC2)n1C] | 31 | (S)-1-[(S)-3-Methyl- 1-({4-[(1-methyl-5- propyl-1H- imidazol-2- yl)methyl]-1- piperidyl}car- bonyl)butyl]-3- isobutyl-2- piperazinone | p- toluenesulfonyl methyl isocyanide; Van Leusen imidazole formation using butyraldehyde and methylamine, deprotonation with nBuLi followed by addition to tert- butyl 4-formyl-1- piperidinecarbox- | CD₃OD: δ 6.66 (s, 1H), 5.55 (m, 1H), 4.47 (t, 1H), 3.52 (s, 3H), 2.54 (t, 2H), 2.02 (m, 1H), 0.97 (m, 15H) | +++ |
| | | | ylate, mesylation | | |
| | | | of alcohol with | | |
| | | | methanesulfonyl | | |
| | | | chloride, | | |
| | | | elimination of | | |
| | | | mesylate at | | |
| | | | 110° C., catalytic | | |
| | | | hydrogenation | | |
| | | | using Pd/C and | | |
| | | | H₂ at 40 psi, BOC | | |
| | | | removal | | |
| 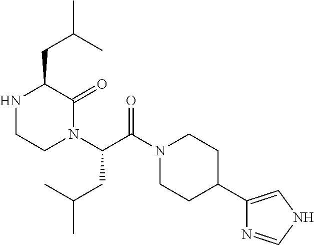 [SMILES: CC(C)C[C@@H]1NCCN([C@@H](CC(C)C)C(=O)N2CCC(c3c[nH]cn3)CC2)C1=O] | 32 | (S)-1-[(S)-1-{[4-(1H- Imidazol-4-yl)-1- piperidyl]carbonyl}- 3-methylbutyl]-3- isobutyl-2- piperazinone | 4-(4-Piperidyl)- 1H-imidazole (Enamine BB) | CD₃OD: δ 7.68 (s, 1H), 6.87 (s, 1H), 5.59 (m, 1H), 4.54 (m, 1H), 2.03 (d, 2H), 0.95 (m, 12H) | ++ |
| 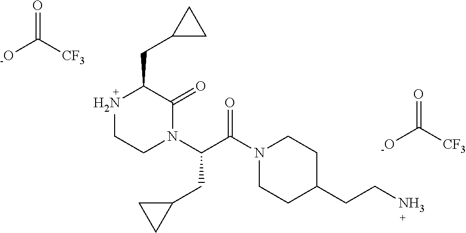 | 33 | (S)-1-{(S)-2-[4-(2- Aminoethyl)-1- piperidyl]-1- (cyclopropylmethyl)- 2-oxoethyl}-3- (cyclopropylmethyl)- 2-piperazinone (2TFA) | 4-[2-(tert- Butoxycarbonyl- amino)ethyl]piper- idine (Combi- Blocks) | CD₃OD: δ 5.54 (m, 1H), 2.99 (m, 2H), 0.76- 0.43 (br m, 5H), 0.31-0.33- 0.096 (br m, 4H) | ++ |
| 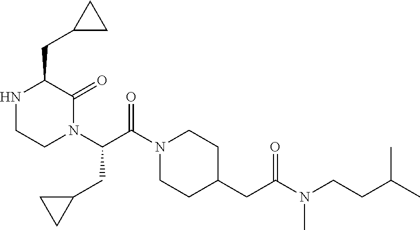 [SMILES: CC(C)CCN(C)C(=O)CC1CCN(C(=O)[C@H](CC2CC2)N2CCN[C@@H](CC3CC3)C2=O)CC1] | 34 | (S)-1-[(S)-2-(4-{2- [N- Methyl(isopentyl) amino]-2- oxoethyl}- 1-piperidyl)-1- (cyclopropylmethyl)- 2-oxoethyl]-3- (cyclopropylmethyl)- 2-piperazinone | (1-tert- Butoxycarbonyl- 4- piperidyl)acetic acid: amide coupling with N- Methyl(isopentyl) amine, BOC removal | CD₃OD: δ 5.57 (m, 1H), 4.50 (t, 1H), 4.09 (t, 1H), 0.96 (m, 6H), 0.47 (m, 4H), 0.14 (m, 4H) | +* |
| 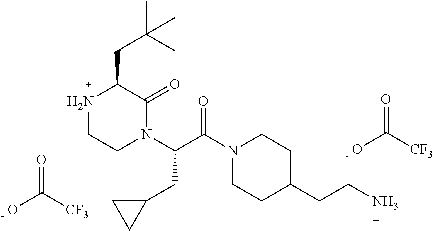 | 35 | (S)-1-{(S)-2-[4-(2- Aminoethyl)-1- piperidyl]-1- (cyclopropylmethyl)- 2-oxoethyl}-3- neopentyl-2- piperazinone (2TFA) | 4-[2-(tert- Butoxy- carbonylamino) ethyl]piperidine (Combi-Blocks) | CD₃OD: δ 5.52 (m, 1H), 4.49 (d, 1H), 1.03 (s, 9H), 0.69 (m, 1H), 0.50 (m, 2H), 0.16 (m, 2H) | ++ |
| 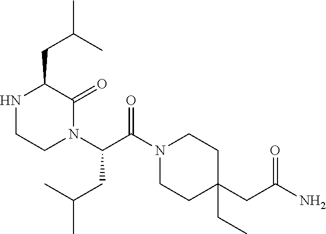 [SMILES: CCC1(CC(N)=O)CCN(C(=O)[C@H](CC(C)C)N2CCN[C@@H](CC(C)C)C2=O)CC1] | 36 | (1-{(S)-2-[(S)-3- Isobutyl-2-oxo-1- piperazinyl]-4- methylvaleryl}-4- ethyl-4- piperidyl)acetamide | 1-Boc-4- piperidone, Meldrum's acid condensation, Grignard reaction, amide formation, tBOC removal | CDCl3: δ, 5.70 (m, 1H), 5.50 (t, 1H), 2.18 (2s, 2H), 0.95, (m, 15H). | ++ |
| 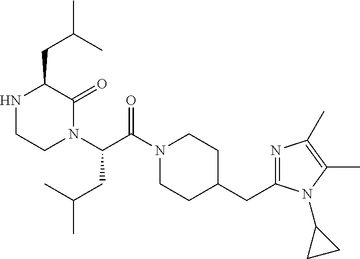 [SMILES: Cc1nc(CC2CCN(C(=O)[C@H](CC(C)C)N3CCN[C@@H](CC(C)C)C3=O)CC2)n(C2CC2)c1C] | 37 | (S)-1-[(S)-1-({4-[(1- Cyclopropyl-4,5- dimethyl-1H- imidazol-2- yl)methyl]-1- piperidyl}carbonyl)- 3-methylbutyl]-3- isobutyl-2- piperazinone | 2-(4- piperidyl)ethanol; tBOC protection, DMP oxidation to aldehyde, imidazole formation with biacetyl, NH₄OAC and cyclopropylamine, BOC removal. | ¹H NMR (CD₃OD) δ 0.96 (m, 12H), 1.17 (m, 6H), 1.65 (m, 8H), 2.71 (m, 3H), 5.56 (m, 1H) | +++ |
| 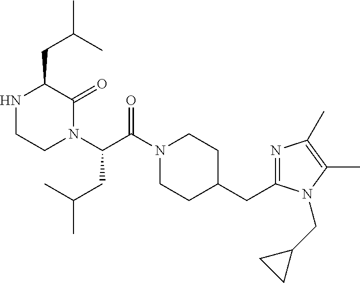 [SMILES: Cc1nc(CC2CCN(C(=O)[C@H](CC(C)C)N3CCN[C@@H](CC(C)C)C3=O)CC2)n(CC2CC2)c1C] | 38 | (S)-1-[(S)-1-[(4-{[1- (Cyclopropylmethyl)- 4,5-dimethyl-1H- imidazol-2- yl]methyl}-1- piperidyl)carbonyl]- 3-methylbutyl]-3- isobutyl-2- piperazinone | 2-(4- piperidyl)ethanol; tBOC protection, DMP oxidation to aldehyde, imidazole formation with biacetyl, NH₄OAc and cyclopropyl- methanamine, BOC removal. | 1H NMR (CD₃OD) δ 0.38 (m, 2H), 0.61 (m, 2H), 0.95 (m, 12H), 2.61 (m, 3H), 3.82 (m, 2H), 5.56 (m, 1H) | +++ |
| 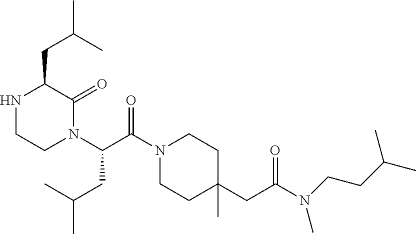 [SMILES: CC(C)CCN(C)C(=O)CC1(C)CCN(C(=O)[C@H](CC(C)C)N2CCN[C@@H](CC(C)C)C2=O)CC1] | 39 | (S)-1-[(S)-1-[(4-{2- [N- Methyl(isopentyl) amino]-2- oxoethyl}- 4-methyl-1- piperidyl)carbonyl]- 3-methylbutyl]-3- isobutyl-2- piperazinone | (4-Methyl-1-tert- butoxycarbonyl- 4- piperidyl)acetic acid (AstaTech) and N,3- dimethylbutan- 1-amine; amide formation, BOC removal. | 1H NMR (CD₃OD) δ 0.96 (m, 18H), 1.15 (m, 3H), 1.62 (m, 14H), 3.56 (m, 7H), 5.56 (m, 1H) | ++ |
| 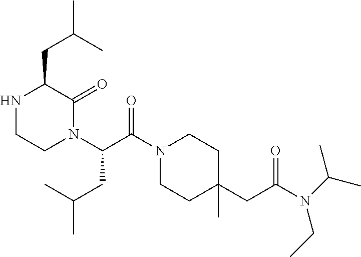 [SMILES: CCN(C(=O)CC1(C)CCN(C(=O)[C@H](CC(C)C)N2CCN[C@@H](CC(C)C)C2=O)CC1)C(C)C] | 40 | (S)-1-[(S)-1-[(4-{2- [N- Ethyl(isopropyl) amino]-2- oxoethyl}-4- methyl-1- piperidyl)carbonyl]- 3-methylbutyl]-3- isobutyl-2- piperazinone | (4-Methyl-1-tert- butoxycarbonyl- 4- piperidyl)acetic acid (AstaTech) and N- Ethyl(isopropyl) amine; amide formation, BOC removal. | ¹H NMR (CD₃OD) δ 0.96 (m, 12H), 1.19 (m, 12H), 1.63 (m, 10H), 2.40 (m, 2H), 5.58 (m, 1H) | +* |
| 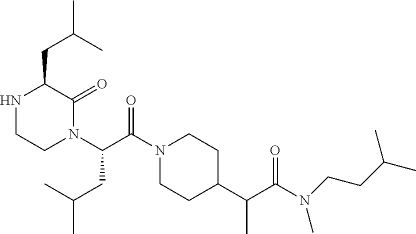 [SMILES: CC(C)CCN(C)C(=O)C(C)C1CCN(C(=O)[C@H](CC(C)C)N2CCN[C@@H](CC(C)C)C2=O)CC1] | 41 | (S)-1-[(S)-1-[(4-{2- [N- Methyl(isopentyl) amino]-1-methyl- 2-oxoethyl}-1- piperidyl)carbonyl]- 3-methylbutyl]-3- isobutyl-2- piperazinone | 2-(1-tert- Butoxycarbonyl- 4-piperidyl)pro- pionic acid (AstaTech) and N,3- dimethylbutan- 1-amine; amide formation, BOC removal. | ¹H NMR (CD₃OD) δ 0.96 (m, 18H), 1.10 (m, 6H), 2.62 (m, 2H), 4.52 (m, 1H), 5.56 (m, 1H) | ++ |
| 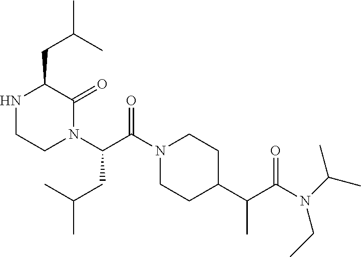 [SMILES: CCN(C(=O)C(C)C1CCN(C(=O)[C@H](CC(C)C)N2CCN[C@@H](CC(C)C)C2=O)CC1)C(C)C] | 42 | (S)-1-[(S)-1-[(4-{2- [N- Ethyl(isopropyl)ami- no]-1-methyl-2- oxoethyl}-1- piperidyl)carbonyl]- 3-methylbutyl]-3- isobutyl-2- piperazinone | 2-(1-tert- Buxoycarbonyl- 4-piperidyl)pro- pionic acid (AstaTech) and N- Ethyl(isopropyl) amine; amide formation, BOC rremoval. | ¹H NMR (CD₃OD) δ 0.96 (m, 14H), 1.17 (m, 16H), 1.68 (m, 10H), 5.56 (m, 1H) | +* |
| 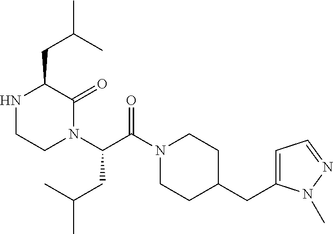 [SMILES: CC(C)C[C@@H]1NCCN([C@@H](CC(C)C)C(=O)N2CCC(Cc3ccnn3C)CC2)C1=O] | 43 | (S)-1-[(S)-3-Methyl- 1-({4-[(2-methyl- 2H-pyrazol-3- yl)methyl]-1- piperidyl}car- bonyl)butyl]-3- isobutyl-2- piperazinone | 1-Methyl-5-[(4- piperidyl)methyl]- 1H-pyraozle (Enamine) | CD₃OD: δ 7.35 (s, 1H), 6.09 (s, 1H), 5.55 (m, 1H), 4.48 (t, 1H), 3.78 (s, 3H), 2.88 (m, 1H), 0.95 (m, 12H) | +++ |
| 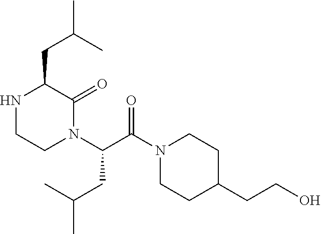 [SMILES: CC(C)C[C@@H]1NCCN([C@@H](CC(C)C)C(=O)N2CCC(CCO)CC2)C1=O] | 44 | (S)-1-[(S)-1-{[4-(2- Hydroxyethyl)-1- piperidyl]carbonyl}- 3-methylbutyl]-3- isobutyl-2- piperazinone | 2-(4- Piperidyl)ethanol | CD₃OD: δ 5.56 (m, 1H), 4.47 (t, 1H), 3.62 (t, 3H), 2.67 (q, 1H), 0.95 (m, 12H) | ++ |
| 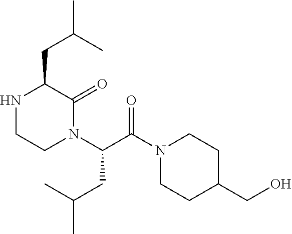 [SMILES: CC(C)C[C@@H]1NCCN([C@@H](CC(C)C)C(=O)N2CCC(CO)CC2)C1=O] | 45 | (S)-1-[(S)-1-{[4- (Hydroxymethyl)- 1- piperidyl]carbonyl}- 3-methylbutyl]-3- isobutyl-2- piperazinone | (4-Piperi- dyl)methanol | CD₃OD: δ 5.57 (m, 1H), 4.51 (t, 1H), 2.67 (q, 1H), 1.13 (m, 2H), 0.95 (m, 12H) | +* |
| 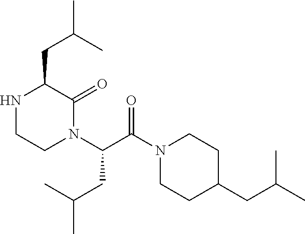 [SMILES: CC(C)CC1CCN(C(=O)[C@H](CC(C)C)N2CCN[C@@H](CC(C)C)C2=O)CC1] | 46 | (S)-1-{(S)-1-[(4- Isobutyl-1- piperidyl)carbonyl]- 3-methylbutyl}-3- isobutyl-2- piperazinone | 4-Isobutylpiperidine (Enamine BB) | CD₃OD: δ 5.56 (m, 1H), 4.46 (t, 1H), 2.66 (q, 1H), 1.87-1.38 (br m, 10H), 1.20-1.02 (br m, 3H), 1.02- 0.72 (br m, 19H) | +++ |
| 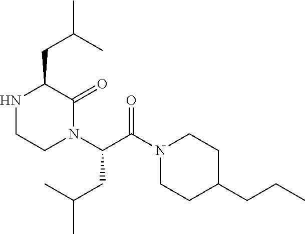 [SMILES: CCCC1CCN(C(=O)[C@H](CC(C)C)N2CCN[C@@H](CC(C)C)C2=O)CC1] | 47 | (S)-1-{(S)-3-Methyl- 1-[(4-propyl-1- piperidyl)car- bonyl]butyl}-3- isobutyl-2- piperazinone | 4- Propylpiperidine | CD₃OD: δ 5.56 (m, 1H), 4.46 (t, 1H), 2.64 (q, 1H), 1.92-1.44 (br m, 9H), 1.44-1.19 (br m, 4H), 1.19- 0.81 (br m, 17H) | +++ |
| 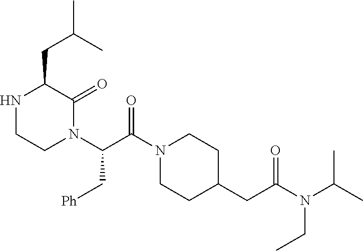 [SMILES: CCN(C(=O)CC1CCN(C(=O)[C@H](Cc2ccccc2)N2CCN[C@@H](CC(C)C)C2=O)CC1)C(C)C] | 48 | (S)-1-[(S)-2-(4-{2- [N- Ethyl(isopropyl)ami- no]-2-oxoethyl}-1- piperidyl)-1-benzyl- 2-oxoethyl]-3- isobutyl-2- piperazinone | (1-tert- Butoxycarbonyl- 4- piperidyl)acetic acid: amide coupling with N- Ethyl(isopropyl) amine, BOC removal | CD₃OD: δ 7.27 (m, 5H), 5.76 (m, 1H), 4.52 (m, 2H), 4.09 (m, 2H), 1.25- 1.07 (br m, 12H), 0.96- 0.80 (m, 8H) | ++ |
| 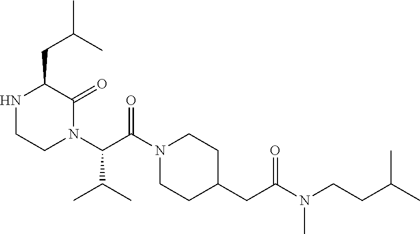 [SMILES: CC(C)CCN(C)C(=O)CC1CCN(C(=O)[C@H](C(C)C)N2CCN[C@@H](CC(C)C)C2=O)CC1] | 49 | (S)-1-[(S)-1-[(4-{2- [N- Methyl(isopentyl) amino]-2- oxoethyl}-1- piperidyl)carbonyl]- 2-methylpropyl]- 3-isobutyl-2- piperazinone | (1-tert- Butoxycarbonyl- 4- piperidyl)acetic acid; amide coupling with N- Methyl(isopentyl) amine, BOC removal | CD₃OD: δ 5.13 (m, 1H), 4.52 (t, 1H), 4.25 (m, 1H), 1.01- 0.84 (br m, 19H) | +* |
| 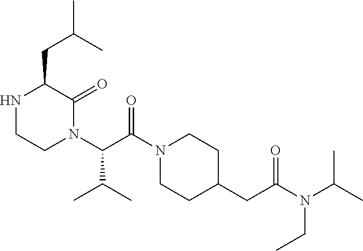 [SMILES: CCN(C(=O)CC1CCN(C(=O)[C@H](C(C)C)N2CCN[C@@H](CC(C)C)C2=O)CC1)C(C)C] | 50 | (S)-1-[(S)-1-[4-{2- [N- Ethyl(isopropyl)ami- no]-2-oxoethyl}-1- piperidyl)carbonyl]- 2-methylpropyl]- 3-isobutyl-2- piperazinone | (1-tert- Butoxycarbonyl- 4- piperidyl)acetic acid: amide coupling with N- Ethyl(isopropyl) amine, BOC removal | CD₃OD: δ 5.13 (m, 1H), 4.55 (m, 2H), 4.22 (m, 2H), 1.26- 1.07 (br m, 12H), 1.03- 0.81 (br m, 14H) | +* |
| 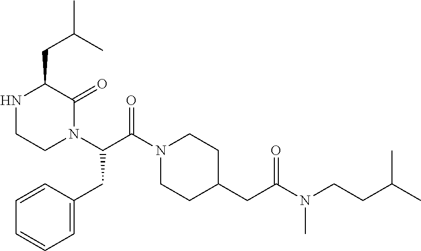 | 51 | (2-(1-((S)-2-((S)-3- isobutyl-2- oxopiperazin-1-yl)- 3-phenyl- propanoyl)piperidin- 4-yl)-N- isopentyl-N- methylacetamide | (1-tert- Butoxycarbonyl- 4- piperidyl)acetic acid; amide coupling with N- Methyl(isopentyl) amine, BOC removal | CD₃OD: δ 7.25 (m, 5H), 5.77 (m, 1H), 4.47 (t, 1H), 4.01 (t, 1H), 1.01-0.81 (m, 14H) | ++ |
| 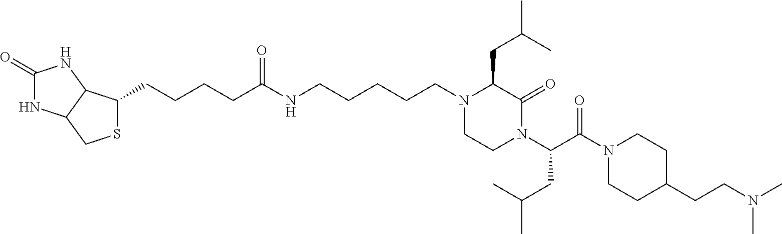 [SMILES: CC(C)C[C@H]1C(=O)N([C@@H](CC(C)C)C(=O)N2CCC(CCN(C)C)CC2)CCN1CCCCCNC(=O)CCCC[C@@H]1SCC2NC(=O)NC21] | 52 | (6S)-6-(5-{5-[(S)-4- [(S)-1-({4-[2- (Dimethylamino)eth- yl]-1- piperidyl}carbonyl)- 3-methylbutyl]-2- isobutyl-3-oxo-1- piperazinyl]pentyla- mino}-5- oxopentyl)-7-thia- 2.4- diazabicyclo[3.3.0] octan-3-one | N,N-Dimethyl[2- (4-piperi- dyl)ethyl]amine (Enamine BB) | CD₃OD: δ 5.55 (m, 1H), 4.49 (m, 2H), 3.19 (m, 6H), 2.28 (s, 6H), 1.91- 1.27 (br m, 25H), 1.0-0.87 (br m, 13H) | ++ |
| 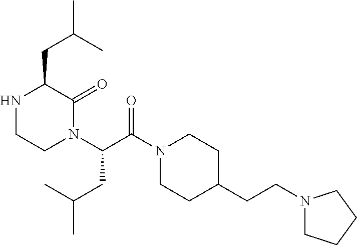 [SMILES: CC(C)C[C@@H]1NCCN([C@@H](CC(C)C)C(=O)N2CCC(CCN3CCCC3)CC2)C1=O] | 53 | (S)-1-[(S)-3-Methyl- 1-({4-[2-(1- pyrrolidinyl)ethyl]- 1- piperidyl}car- bonyl)butyl]-3- isobutyl-2- piperazinone | tert-Butyl 4-(2- hydroxyethyl)-1- piperidinecarbox- ylate, oxidation to aldehyde, reductive amination with pyrrolidine, BOC deprotection | CD₃OD: δ 5.56 (m, 1H), 4.48 (t, 1H), 2.69 (m, 6H), 1.71 (m, 16H), 0.96 (m, 12H) | ++++ |
| 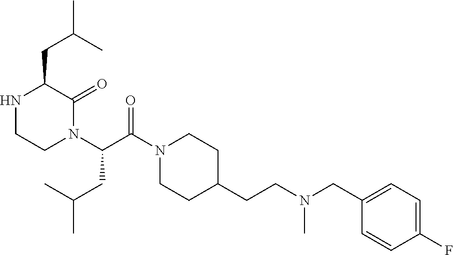 [SMILES: CC(C)C[C@@H]1NCCN([C@@H](CC(C)C)C(=O)N2CCC(CCN(C)Cc3ccc(F)cc3)CC2)C1=O] | 54 | (S)-1-[(S)-1-{[4-(2- {N-Methyl[(p- fluorophenyl)meth- yl]amino}ethyl)-1- piperidyl]carbonyl}- 3-methylbutyl]-3- isobutyl-2- piperazinone | tert-Butyl 4-(2- hydroxyethyl)-1- piperidinecarbox- ylate, oxidation to aldehyde, reductive amination with N-Methyl[(p- fluorophenyl)meth- yl]amine, BOC removal | CD₃OD: δ 7.35 (m, 2H), 7.07 (m, 2H), 5.58 (m, 1H), 4.46 (t, 1H), 3.53 (s, 2H), 2.23 (s, 3H), 0.97 (m, 12H) | ++++ |
| 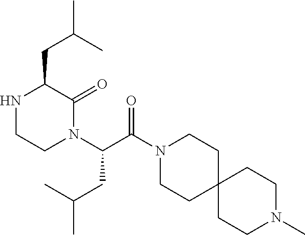 [SMILES: CC(C)C[C@@H]1NCCN([C@@H](CC(C)C)C(=O)N2CCC3(CCN(C)CC3)CC2)C1=O] | 55 | (S)-1-[(S)-3-Methyl- 1-{(9-methyl-3,9- diaza-3- spiro[5.5]undecyl)car- bonyl}butyl]-3- isobutyl-2- piperazinone | 3-Methyl-3,9- diazaspiro[5.5]un- decane (Enamine BB) | CD₃OD: δ 5.56 (m, 1H), 2.50 (m, 4H), 2.33 (s, 3H), 1.62 (m, 14H), 0.95 (m, 12H) | +++ |
| 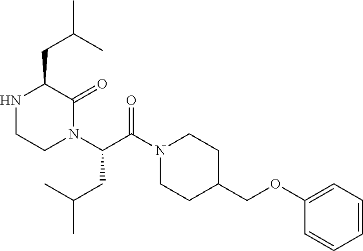 [SMILES: CC(C)C[C@@H]1NCCN([C@@H](CC(C)C)C(=O)N2CCC(COc3ccccc3)CC2)C1=O] | 56 | (S)-1-[(S)-3-Methyl- 1-{[4- (phenoxymethyl)- 1- piperidyl]car- bonyl}butyl]-3- isobutyl-2- piperazinone | 4- (Phenoxymeth- yl)piperidine (Enamine BB) | CD₃OD: δ 7.28 (t, 2H), 6.93 (m, 3H), 5.62 (m, 1H), 4.57 (t, 1H), 3.87 (d, 2H), 0.99 (m, 12H) | +++ |
| 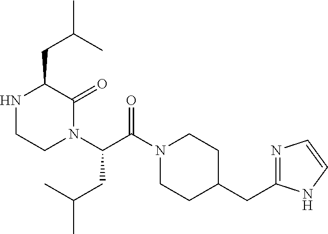 [SMILES: CC(C)C[C@@H]1NCCN([C@@H](CC(C)C)C(=O)N2CCC(Cc3ncc[nH]3)CC2)C1=O] | 57 | (S)-1-[(S)-1-({4- [(1H-Imidazol-2- yl)methyl]-1- piperidyl}carbonyl)- 3-methylbutyl]-3- isobutyl-2- piperazinone | 2-[(4- Piperidyl)methyl]- 1H-imidazole (Enamine BB) | CD₃OD: δ 6.86 (s, 2H), 5.45 (m, 1H), 4.37 (t, 1H), 2.57 (d, 2H), 0.85 (m, 12H) | +++ |
| 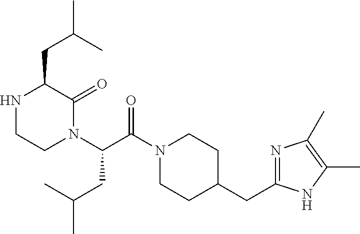 [SMILES: Cc1nc(CC2CCN(C(=O)[C@H](CC(C)C)N3CCN[C@@H](CC(C)C)C3=O)CC2)[nH]c1C] | 58 | (S)-1-[(S)-1-({4- [(4,5-Dimethyl-1H- imidazol-2- yl)methyl]-1- piperidyl}carbonyl)- 3-methylbutyl]-3- isobutyl-2- piperazinone | tert-Butyl 4-(2- hydroxyethyl)-1- piperidinecarbox- ylate, oxidation to aldehyde, imidazole formation using ammonium acetate and 2,3- butanedione, BOC deprotection | CD₃OD: δ 5.37 (m, 1H), 4.29 (t, 1H), 2.36 (d, 2H), 1.90 (s, 6H), 0.77 (m, 12H) | +++ |
| 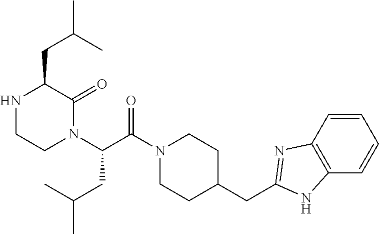 [SMILES: CC(C)C[C@@H]1NCCN([C@@H](CC(C)C)C(=O)N2CCC(Cc3nc4ccccc4[nH]3)CC2)C1=O] | 59 | (S)-1-[(S)-1-({4- [(1,3-Benzimidazol- 2-yl)methyl]-1- piperidyl}carbonyl)- 3-methylbutyl]-3- isobutyl-2- piperazinone | 2-[(4- Piperidyl)methyl]- 1,3- benzimidazole (Enamine BB) | CD₃OD: δ 7.45 (dd, 2H), 7.14 (dd, 2H), 5.49 (m, 1H), 4.43 (t, 1H), 2.79 (d, 2H), 0.89 (m, 12H) | ++ |
| 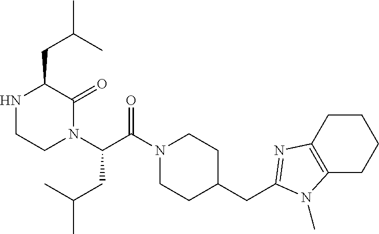 [SMILES: CC(C)C[C@@H]1NCCN([C@@H](CC(C)C)C(=O)N2CCC(Cc3nc4c(n3C)CCCC4)CC2)C1=O] | 60 | (S)-1-[(S)-3-Methyl- 1-({4-[(1-methyl- 1,3-diaza-4,5,6,7- tetrahydro-1H- inden-2-yl)methyl]- 1- piperidyl}car- bonyl)butyl]-3- isobutyl-2- piperazinone | tert-Butyl 4-(2- hydroxyethyl)-1- piperidinecarbox- ylate, oxidation to aldehyde, imidazole formation using ammonium acetate, methylamine and 1,2- cyclohexanedione, BOC deprotection | CD₃OD: δ 5.54 (m, 1H), 4.46 (t, 1H), 3.43 (s, 3H), 2.47 (d, 2H), 0.94 (m, 12H) | +++ |
| 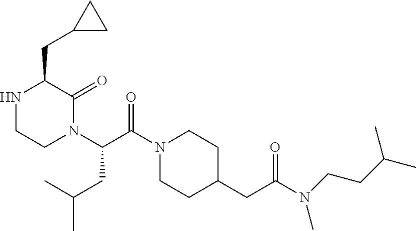 [SMILES: CC(C)CCN(C)C(=O)CC1CCN(C(=O)[C@H](CC(C)C)N2CCN[C@@H](CC3CC3)C2=O)CC1] | 61 | (S)-1-[(S)-1-[(4-{2- [N- methyl(isopentyl) amino]-2- oxoethyl}-1- piperidyl)carbonyl]- 3-methylbutyl]-3- (cyclopropylmethyl)- 2-piperazinone | (1-tert- Butoxycarbonyl- 4- piperidyl)acetic acid and N,3- dimethylbutan- 1-amine; amide formation, BOC removal. | 1H NMR (CD₃OD) δ 0.16 (m, 2H), 0.49 (m, 2H), 0.96 (m, 12H), 1.15 (m, 2H), 1.60 (m, 11H), 2.33 (m, 2H), 4.08 (m, 1H), 5.56 (m, 1H) | ++ |
| 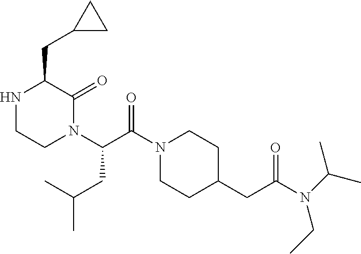 [SMILES: CCN(C(=O)CC1CCN(C(=O)[C@H](CC(C)C)N2CCN[C@@H](CC3CC3)C2=O)CC1)C(C)C] | 62 | (S)-1-[(S)-1-[(4-{2- [N- Ethyl(isopropyl)ami- no]-2-oxoethyl}-1- piperidyl)carbonyl]- 3-methylbutyl]-3- (cyclopropylmethyl)- 2-piperazinone | (1-tert- Butoxycarbonyl- 4- piperidyl)acetic acid and 2- (isopropylamino) ethan-1-ylium; amide formation, BOC removal. | ¹H NMR (CD₃OD) δ 0.15 (m, 2H), 0.47 (m, 2H), 0.96 (m, 6H), 1.18 (m, 10H), 1.66 (m, 7H), 2.34 (m, 2H), 5.57 (m, 1H) | ++ |
| 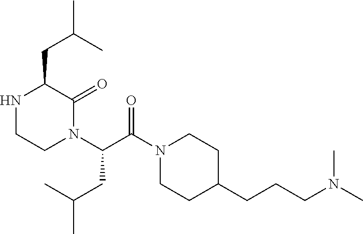 [SMILES: CC(C)C[C@@H]1NCCN([C@@H](CC(C)C)C(=O)N2CCC(CCCN(C)C)CC2)C1=O] | 63 | (S)-1-[(S)-1-({4-[3- (Dimethylamino)pro- pyl]-1- piperidyl}carbonyl)- 3-methylbutyl]-3- isobutyl-2- piperazinone | N,N-dimethyl-3- (piperidin-4- yl)propan-1- amine (Enamine BB) | ¹H NMR (CD₃OD) ∂ 0.99 (m, 14H), 1.29 (m, 2H), 1.67 (m, 11H), 2.42 (S, 6H), 2.52 (m, 2H), 4.47 (m, 1H), 5.57 (m, 1H) | +++ |
| 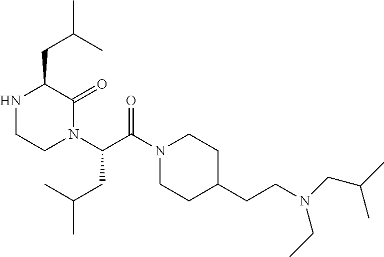 [SMILES: CCN(CCC1CCN(C(=O)[C@H](CC(C)C)N2CCN[C@@H](CC(C)C)C2=O)CC1)CC(C)C] | 64 | (S)-1-[(S)-1-[(4-{2- [N- Ethyl(isobutyl)ami- no]ethyl}-1- piperidyl)carbonyl]- 3-methylbutyl]-3- isobutyl-2- piperazinone | N-ethyl-2- methyl-N-(2- (piperidin-4- yl)ethyl)propan- 1-amine (Enamine BB) | ¹H NMR (CD₃OD) δ 1.00 (m, 24H), 1.61 (m, 14H), 2.25 (m, 2H), 2.57 (m, 5H), 4.08 (m, 1H), 5.56 (m, 1H) | +++ |
| 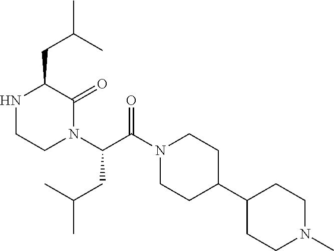 [SMILES: CC(C)C[C@@H]1NCCN([C@@H](CC(C)C)C(=O)N2CCC(C3CCN(C)CC3)CC2)C1=O] | 65 | (S)-1-{(S)-3-Methyl- 1-[(1′-methyl-4,4′- bipiperidyl-1- yl)carbonyl]butyl}- 3-isobutyl-2- piperazinone | 1-methyl-4,4′- bipiperidine dihydrochloride (Matrix Scientific) | ¹H NMR (CD₃OD) δ 0.97 (m, 12H), 1.14 (m, 4H), 1.56 (m, 14H), 2.39 (s, 3H), 5.56 (m, 1H) | +++ |
| 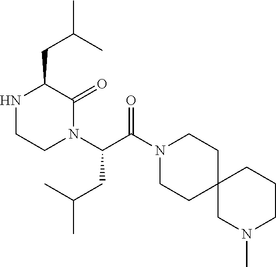 [SMILES: CC(C)C[C@@H]1NCCN([C@@H](CC(C)C)C(=O)N2CCC3(CCCN(C)C3)CC2)C1=O] | 66 | (S)-1-[(S)-3-Methyl- 1-{(2-methyl-2,9- diaza-9- spiro[5.5]undecyl)car- bonyl}butyl]-3- isobutyl-2- piperazinone | 2-methyl-2,9- diazaspiro[5.5]un- decane dihydrochloride (Enamine BB) | ¹H NMR (CD₃OD) δ 0.97 (m, 12H), 1.56 (m, 12H), 3.12 (m, 1H), 5.56 (m, 1H) | +++ |
| 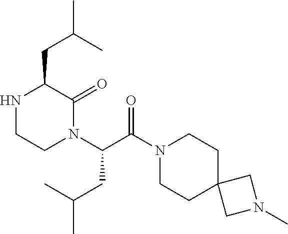 [SMILES: CC(C)C[C@@H]1NCCN([C@@H](CC(C)C)C(=O)N2CCC3(CC2)CN(C)C3)C1=O] | 67 | (S)-1-[(S)-3-Methyl- 1-{(2-methyl-2,7- diaza-7- spiro[3.5]nonyl)car- bonyl}butyl]-3- isobutyl-2- piperazinone | 2-methyl-2,7- diaza- spiro[3.5]nonane dihydrochloride (AstaTech) | ¹H NMR (CD₃OD) δ 0.96 (m, 12H), 1.68, m, 10H), 2.68 (s, 3H), 3.64 (m, 6H), 5.54 (m, 1H) | ++ |
| 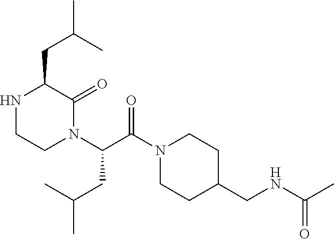 [SMILES: CC(=O)NCC1CCN(C(=O)[C@H](CC(C)C)N2CCN[C@@H](CC(C)C)C2=O)CC1] | 68 | (S)-1-[(S)-1-{[4- (Acetylaminometh- yl)-1- piperidyl]car- bonyl}- 3-methylbutyl]-3- isobutyl-2- piperazinone | 1-{[(4- Piperidyl)meth- yl]amino}-1- ethanone (Enamine BB) | CD₃OD: δ 5.56 (m, 1H), 4.48 (t, 1H), 2.66 (q, 1H), 1.94 (s, 3H), 1.26-0.80 (br m, 15H) | +* |
| 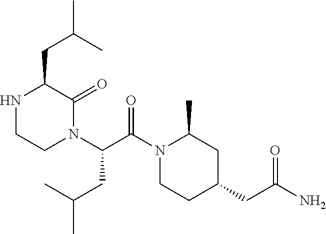 [SMILES: CC(C)C[C@@H]1NCCN([C@@H](CC(C)C)C(=O)N2CC[C@@H](CC(N)=O)C[C@@H]2C)C1=O] | 69 | [(2S,4R)-1-{(S)-2- [(S)-3-Isobutyl-2- oxo-1-piperazinyl]- 4-methylvaleryl}-2- methyl-4- piperidyl]acetamide | tert-Butyl (S)-2- methyl-4-oxo-1- piperidinecarbox- ylate (AstaTech); Wittig reaction with methyl (triphenylphos- phoranyli- dene)acetate, catalytic hydrogenation using Pd/C and H₂, conversion of ester to amide using NH₄OH, | CD₃OD: δ 5.60- 4.43 (br m, 1H), 2.10 (m, 2H), 1.92-1.42 (br m, 8H), 1.42-1.05 (br m, 5H), 0.95 (m, 12H) | +++ |
| | | | BOC removal, | | |
| | | | diastereomeric | | |
| | | | resolution by | | |
| | | | column | | |
| | | | chromatography | | |
| 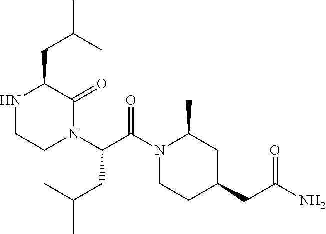 [SMILES: CC(C)C[C@@H]1NCCN([C@@H](CC(C)C)C(=O)N2CC[C@H](CC(N)=O)C[C@@H]2C)C1=O] | 70 | [(2S,4S)-1-{(S)-2- [(S)-3-Isobutyl-2- oxo-1-piperazinyl]- 4-methylvaleryl}-2- methyl-4- piperidyl]acetamide | tert-Butyl (S)-2- methyl-4-oxo-1- piperidinecarbox- ylate (AstaTech); Wittig reaction with methyl (triphenylphos- phoranyli- dene)acetate, catalytic hydrogenation using Pd/C and H₂, conversion of ester to amide using NH₄OH, | CD₃OD: δ 5.49 (t, 1H), 2.09- 1.69 (br m, 5H), 1.69-1.42 (br m, 4H), 1.42-1.09 (br m, 6H), 0.95 (m, 12H) | ++ |
| | | | BOC removal, | | |
| | | | diastereomeric | | |
| | | | resolution by | | |
| | | | column | | |
| | | | chromatography | | |
| 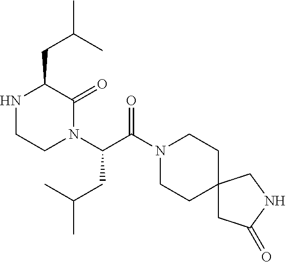 [SMILES: CC(C)C[C@@H]1NCCN([C@@H](CC(C)C)C(=O)N2CCC3(CC2)CNC(=O)C3)C1=O] | 71 | (S)-1-[(S)-3-Methyl- 1-{(3-oxo-2,8- diaza-8- spiro[4.5]decyl)car- bonyl}butyl]-3- isobutyl-2- piperazinone | 2,8-Diaza-3- spiro[4.5]decanone (Combi- Blocks) | CD₃OD: δ 5.59 (t, 1H), 2.30 (d, 2H), 1.82 (m, 2H), 1.74-1.40 (br m, 8H), 0.97 (m, 12H) | +* |
| 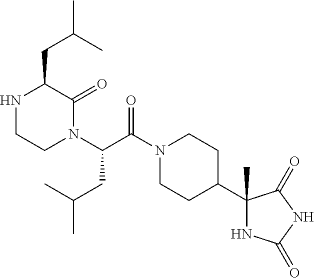 [SMILES: CC(C)C[C@@H]1NCCN([C@@H](CC(C)C)C(=O)N2CCC([C@@]3(C)NC(=O)NC3=O)CC2)C1=O] | 72 | (R)-5-(1-{(S)-2-[(S)- 3-Isobutyl-2-oxo-1- piperazinyl]-4- methylvaleryl}-4- piperidyl)-5- methyl-2,4- imidazolidinedione | 5-Methyl-5-(4- piperidyl)-2,4- imidazolidinedione (Enamine BB); diastereomeric resolution by column chromatography | CD₃OD: δ 5.56 (m, 1H), 4.57 (t, 1H), 2.60 (m, 1H), 1.39 (s, 3H), 0.95 (m 12H) | +* |
| 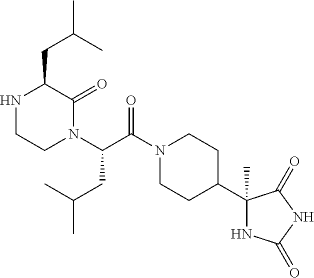 [SMILES: CC(C)C[C@@H]1NCCN([C@@H](CC(C)C)C(=O)N2CCC([C@]3(C)NC(=O)NC3=O)CC2)C1=O] | 73 | (S)-5-(1-{(S)-2-[(S)- 3-Isobutyl-2-oxo-1- piperazinyl]-4- methylvaleryl}-4- piperidyl)-5- methyl-2,4- imidazolidinedione | 5-Methyl-5-(4- piperidyl)-2,4- imidazolidinedione (Enamine BB); diastereomeric resolution by column chromatography | CD₃OD: δ 5.67- 5.47 (br m, 1H), 4.58 (t, 1H), 2.61 (m, 1H), 1.38 (s, 3H), 0.95 (m, 12H) | +* |
| 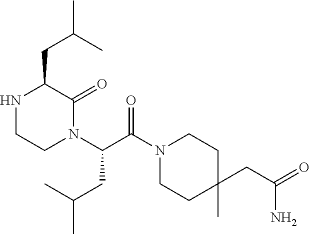 [SMILES: CC(C)C[C@@H]1NCCN([C@@H](CC(C)C)C(=O)N2CCC(C)(CC(N)=O)CC2)C1=O] | 74 | (1-{(S)-2-[(S)-3- Isobutyl-2-oxo-1- piperazinyl]-4- methylvaleryl}-4- methyl-4- piperidyl)acetamide | (4-Methyl-1-tert- butoxycarbonyl- 4- piperidyl)acetic acid (AstaTech); amide coupling with NH₄OH, BOC removal | CD₃OD: δ 5.56 (m, 1H), 2.19 (d, 2H), 1.14 (m, 3H), 0.95 (m, 12H) | ++ |
| 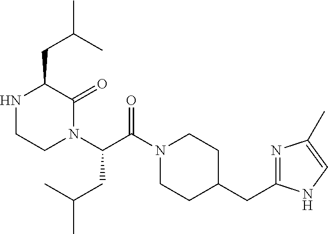 [SMILES: Cc1c[nH]c(CC2CCN(C(=O)[C@H](CC(C)C)N3CCN[C@@H](CC(C)C)C3=O)CC2)n1] | 75 | (S)-1-[(S)-3-Methyl- 1-({4-[(4-methyl- 1H-imidazol-2- yl)methyl]-1- piperidyl}car- bonyl)butyl]-3- isobutyl-2- piperazinone | tert-Butyl 4-(2- hydroxyethyl)-1- piperidinecarbox- ylate, oxidation to aldehyde, imidazole formation using ammonium acetate and pyruvaldehyde, BOC deprotection | CD₃OD: δ 6.59 (s, 1H), 5.52 (m, 1H), 4.45 (t, 1H), 2.57 (d, 2H), 0.93 (m, 12H) | +++ |
| 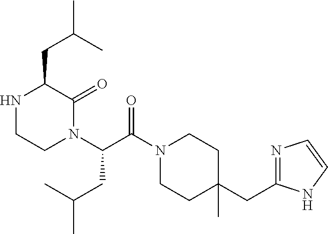 [SMILES: CC(C)C[C@@H]1NCCN([C@@H](CC(C)C)C(=O)N2CCC(C)(Cc3ncc[nH]3)CC2)C1=O] | 76 | (S)-1-[(S)-1-({4- [(1H-Imidazol-2- yl)methyl]-4- methyl-1- piperidyl}carbonyl)- 3-methylbutyl]-3- isobutyl-2- piperazinone | tert-Butyl 4-(2- hydroxyethyl)-4- methyl-1- piperidinecarbox- ylate (Combi- Blocks), oxidation to aldehyde, imidazole formation using ammonium acetate and glyoxal, BOC deprotection | CD₃OD: δ 6.89 (s, 2H), 5.44 (m, 1H), 4.45 (t, 1H), 1.46 (m, 10H), 0.94 (s, 3H), 0.84 (m, 12H) | +++ |
| 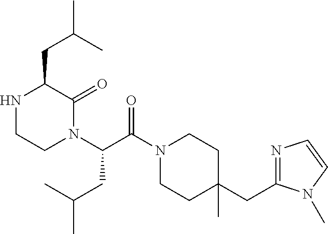 [SMILES: CC(C)C[C@@H]1NCCN([C@@H](CC(C)C)C(=O)N2CCC(C)(Cc3nccn3C)CC2)C1=O] | 77 | (S)-1-[(S)-3-Methyl- 1-({4-methyl-4-[(1- methyl-1H- imidazol-2- yl)methyl]-1- piperidyl}car- bonyl)butyl]-3- isobutyl-2- piperazinone | tert-Butyl 4-(2- hydroxyethyl)-4- methyl-1- piperidinecarbox- ylate (Combi- Blocks), oxidation to aldehyde, imidazole formation using ammonium acetate, methylamine and glyoxal), BOC | CDCl₃: δ 7.01 (s, 1H), 6.90 (s, 1H), 5.56 (m, 1H), 3.66 (s, 3H), 1.09 (s, 3H), 0.96 (m, 12H) | +++ |
| | | | deprotection | | |
| 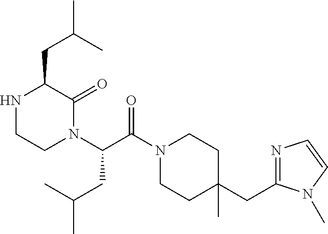 [SMILES: CC(C)C[C@@H]1NCCN([C@@H](CC(C)C)C(=O)N2CCC(C)(Cc3nccn3C)CC2)C1=O] | 78 | (S)-1-[(S)-1-({4- [(4,5-Diethyl-1H- imidazol-2- yl)methyl]-1- piperidyl}carbonyl)- 3-methylbutyl]-3- isobutyl-2- piperazinone | tert-Butyl 4-(2- hydroxyethyl)-1- piperidinecarbox- yalte, oxidation to aldehyde, imidazole formation using ammonium acetate, and 3,4- hexanedione, BOC deprotection | CD₃OD: δ 5.53 (m, 1H), 2.66 (q, 4H), 1.25 (t, 6H), 1.01 (m, 12H) | ++ |
| 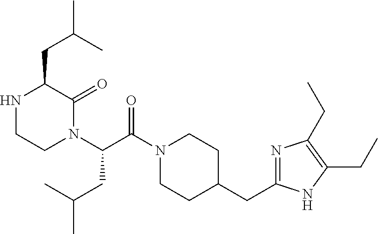 [SMILES: CCc1nc(CC2CCN(C(=O)[C@H](CC(C)C)N3CCN[C@@H](CC(C)C)C3=O)CC2)[nH]c1CC] | 79 | (S)-1-[(S)-1-({4- [(4,5-Diethyl-1- methyl-1H- imidazol-2- yl)methyl]-1- piperidyl}carbonyl)- 3-methylbutyl]-3- isobutyl-2- piperazinone | tert-Butyl 4-(2- hydroxyethyl)-1- piperidinecarbox- ylate, oxidation to aldehyde, imidazole formation using ammonium acetate, methylamine and 3,4- hexanedione, BOC deprotection | CD₃OD: δ 5.54 (m, 1H), 3.73 (s, 3H), 2.69 (m, 4H), 1.22 (m, 6H), 0.99 (m, 12H) | +++ |
| 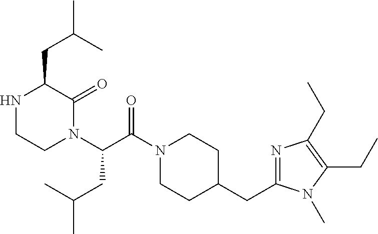 [SMILES: CCc1nc(CC2CCN(C(=O)[C@H](CC(C)C)N3CCN[C@@H](CC(C)C)C3=O)CC2)n(C)c1CC] | 80 | (S)-1-[(S)-1-({4-[(5- Ethyl-1-methyl-1H- imidazol-2- yl)methyl]-1- piperidyl}carbonyl)- 3-methylbutyl]-3- isobutyl-2- piperazinone | p- toluenesulfonyl methyl isocyanide; Van Leusen imidazole formation using propionaldehyde and methylamine, deprotonation with nBuLi followed by addition to tert- butyl 4-formyl-1- piperidinecarbox- | CD₃OD: δ 6.62 (s, 1H), 5.55 (m, 1H), 4.48 (t, 1H), 3.51 (s, 3H), 2.57 (q, 2H), 1.25 (t, 3H), 0.95 (m, 12H) | +++ |
| | | | ylate, mesylation | | |
| | | | of alcohol, | | |
| | | | elimination of | | |
| | | | mesylate by | | |
| | | | heat, | | |
| | | | hydrogenation, | | |
| | | | BOC | | |
| | | | deprotection | | |
| 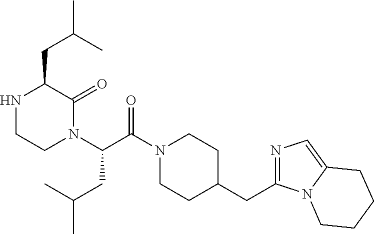 [SMILES: CC(C)C[C@@H]1NCCN([C@@H](CC(C)C)C(=O)N2CCC(Cc3ncc4n3CCCC4)CC2)C1=O] | 81 | (S)-1-[(S)-1-({4- [(2,3a-Diaza- 4,5,6,7- tetrahydroinden-3- yl)methyl]-1- piperidyl}carbonyl)- 3-methylbutyl]-3- isobutyl-2- piperazinone | 2,3a-Diaza- 4,5,6,7- tetrahydroindene (Enamine BB), deprotonation with nBuLi followed by addition to tert- butyl 4-formyl-1- piperidinecarbox- ylate, mesylation of alcohol, elimination of mesylate by heat, | CD₃OD: δ 6.64 (s, 1H), 5.56 (m, 1H), 4.48 (t, 1H), 3.92 (t, 2H), 2.76 (t, 2H), 2.64 (d, 2H), 0.96 (m, 12H) | +++ |
| | | | hydrogenation, | | |
| | | | BOC | | |
| | | | deprotection | | |
| 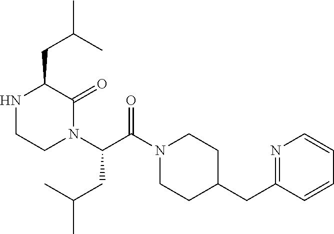 [SMILES: CC(C)C[C@@H]1NCCN([C@@H](CC(C)C)C(=O)N2CCC(Cc3ccccn3)CC2)C1=O] | 82 | (S)-1-[(S)-3-Methyl- 1-({4-[(2- pyridyl)methyl]-1- piperidyl}car- bonyl)butyl]-3- isobutyl-2- piperazinone | 2-[(4- Piperidyl)meth- yl]pyridine (Enamine BB) | CD₃OD: δ 8.47 (s, 1H), 7.78 (t, 1H), 7.30 (m, 2H), 5.57 (m, 1H), 4.48 (t, 1H), 2.76 (d, 2H), 0.97 (m, 12H) | ++ |
| 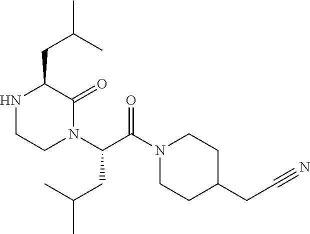 [SMILES: CC(C)C[C@@H]1NCCN([C@@H](CC(C)C)C(=O)N2CCC(CC#N)CC2)C1=O] | 83 | (1-{(S)-2-[(S)-3- Isobutyl-2-oxo-1- piperazinyl]-4- methylvaleryl}-4- piperidyl)acetonitrile | (4- Piperidyl)acetoni- trile (Enamine BB) | CD₃OD: δ 5.63 (m, 1H), 4.59 (t, 1H), 2.55 (d, 2H), 1.01 (m, 12H) | +++ |
| 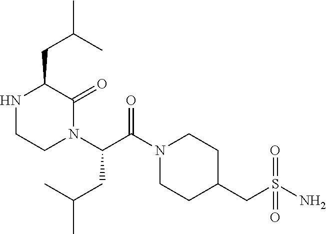 [SMILES: CC(C)C[C@@H]1NCCN([C@@H](CC(C)C)C(=O)N2CCC(CS(N)(=O)=O)CC2)C1=O] | 84 | (1-{(S)-2-[(S)-3- Isobutyl-2-oxo-1- piperazinyl]-4- methylvaleryl}-4- piperidyl)methane- sulfonamide | (4- Piperidyl)methane- sulfonamide (AstaTech) | CD₃OD: δ 5.66 (m, 1H), 4.55 (m, 1H), 2.09 (d, 2H), 1.04 (m, 12H) | +* |
| 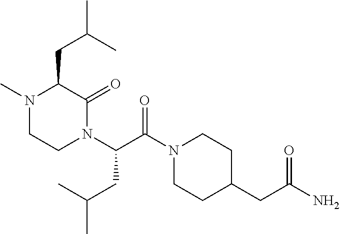 [SMILES: CC(C)C[C@H]1C(=O)N([C@@H](CC(C)C)C(=O)N2CCC(CC(N)=O)CC2)CCN1C] | 85 | (1-{(S)-2-[(S)-3- Isobutyl-4-methyl- 2-oxo-1- piperazinyl]-4- methylvaleryl}-4- piperidyl)acetamide | (4-Piperidyl)acet- amide (Enamine BB) | CDCl₃: δ 5.53 (m, 1H), 4.17 (d, 1H), 3.10- 2.87 (br m, 2H), 2.33 (m, 3H), 1.00-0.76 (br m, 12H) | ++ |
| 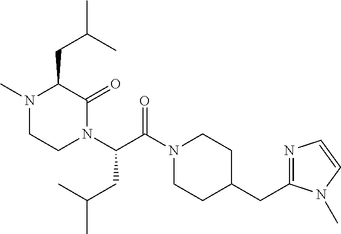 [SMILES: CC(C)C[C@H]1C(=O)N([C@@H](CC(C)C)C(=O)N2CCC(Cc3nccn3C)CC2)CCN1C] | 86 | (S)-1-[(S)-3-Methyl- 1-({4-[(1-methyl- 1H-imidazol-2- yl)methyl]-1- piperidyl}car- bonyl)butyl]-3- isobutyl-4- methyl-2- piperazinone | 1-methyl-2-[(4- piperidyl)methyl]- 1H-imidazole (Enamine BB) | CD₃OD: δ 6.97 (s, 1H), 6.85 (s, 1H), 5.52 (m, 1H), 4.47 (t, 1H), 3.64 (s, 3H), 2.36 (m, 3H), 1.03-0.81 (br m, 12H) | +++ |
| 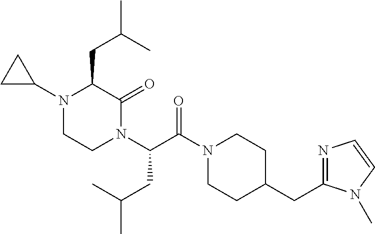 [SMILES: CC(C)C[C@@H](C(=O)N1CCC(Cc2nccn2C)CC1)N1CCN(C2CC2)[C@@H](CC(C)C)C1=O] | 87 | (S)-1-[(S)-3-Methyl- 1-({4-[(1-methyl- 1H-imidazol-2- yl)methyl]-1- piperidyl}car- bonyl)butyl]-4- cyclopropyl-3- isobutyl-2- piperazinone | 1-methyl-2-[(4- piperidyl)methyl]- 1H-imidazole (Enamine BB) | CD₃OD: δ 6.96 (s, 1H), 6.84 (s, 1H), 5.58 (m, 1H), 4.47 (m, 1H), 3.63 (s, 3H), 0.93 (m, 12H), 0.66- 0.33 (br m, 4H) | +++ |
| 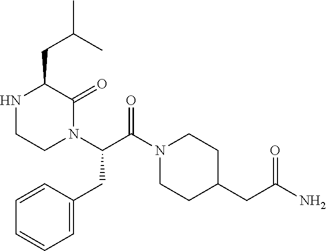 [SMILES: CC(C)C[C@@H]1NCCN([C@@H](Cc2ccccc2)C(=O)N2CCC(CC(N)=O)CC2)C1=O] | 88 | (1-{(S)-2-[(S)-3- Isobutyl-2-oxo-1- piperazinyl]-3- phenylpropionyl}- 4- piperidyl)acetamide | (4-Piperidyl)acet- amide (Enamine BB) | CD₃OD: δ 7.35- 7.15 (br m, 5H), 5.86-5.67 (br m, 1H), 4.46 (t, 1H), 2.63 (t, 1H), 0.93-0.81 (br m, 6H) | ++ |
| 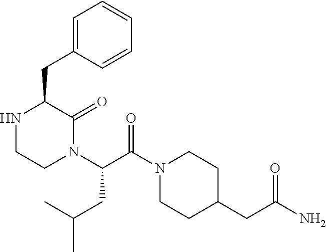 [SMILES: CC(C)C[C@@H](C(=O)N1CCC(CC(N)=O)CC1)N1CCN[C@@H](Cc2ccccc2)C1=O] | 89 | (1-{(S)-2-[(S)-3- Benzyl-2-oxo-1- piperazinyl]-4- methylvaleryl}-4- piperidyl)acetamide | (4-Piperidyl)acet- amide (Enamine BB) | CD₃OD: δ 7.39- 7.16 (br m, 5H), 5.58 (m, 1H), 4.46 (t, 1H), 2.68 (q, 1H), 0.94 (m, 6H) | +* |
| 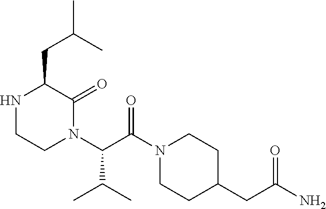 [SMILES: CC(C)C[C@@H]1NCCN([C@H](C(=O)N2CCC(CC(N)=O)CC2)C(C)C)C1=O] | 90 | (1-{(S)-2-[(S)-3- Isobutyl-2-oxo-1- piperazinyl]-3- methylbutyryl}-4- piperidyl)acetamide | (4-Piperidyl)acet- amide (Enamine BB) | CD₃OD: δ 5.12 (m, 1H), 4.52 (t, 1H), 2.69 (q, 1H), 2.33 (m, 1H), 1.00-0.78 (br m, 12H) | ND |
| 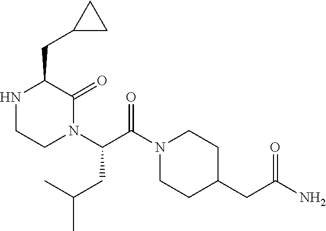 [SMILES: CC(C)C[C@@H](C(=O)N1CCC(CC(N)=O)CC1)N1CCN[C@@H](CC2CC2)C1=O] | 91 | (1-{(S)-2-[(S)-3- (Cyclopropylmethyl)- 2-oxo-1- piperazinyl]-4- methylvaleryl}-4- piperidyl)acetamide | (4-Piperidyl)acet- amide (Enamine BB) | CD₃OD: δ 5.57 (m, 1H), 4.47 (t, 1H), 2.69 (q, 1H), 0.96 (m, 6H), 0.85 (m, 1H), 0.58-0.41 (br m, 2H), 0.26-0.06 (br m, 2H) | +* |
| 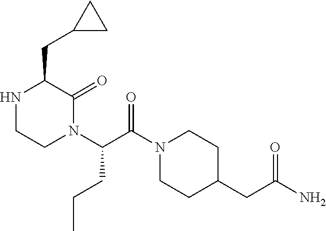 [SMILES: CCC[C@@H](C(=O)N1CCC(CC(N)=O)CC1)N1CCN[C@@H](CC2CC2)C1=O] | 92 | (1-{(S)-2-[(S)-3- (Cyclopropylmethyl)- 2-oxo-1- piperazinyl]valeryl}- 4- piperidyl)acetamide | (4-Piperidyl)acet- amide (Enamine BB) | CD₃OD: δ 5.46 (m, 1H), 4.48 (t, 1H), 2.69 (q, 1H), 1.43-1.01 (br m, 5H), 0.95 (m, 3H), 0.85 (m, 1H), 0.59-0.39 (br m, 2H), 0.26- 0.06 (br m, 2H) | +* |
| 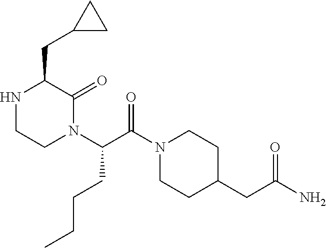 [SMILES: CCCC[C@@H](C(=O)N1CCC(CC(N)=O)CC1)N1CCN[C@@H](CC2CC2)C1=O] | 93 | (1-{(S)-2-[(S)-3- (Cyclopropylmethyl)- 2-oxo-1- piperazinyl]hexa- noyl}-4- piperidyl)acetamide | (4-Piperidyl)acet- amide (Enamine BB) | CD₃OD: δ 5.45 (m, 1H), 4.48 (t, 1H), 2.69 (q, 1H), 1.53-1.00 (br m, 7H), 1.00-0.75 (br m, 4H), 0.59- 0.39 (br m, 2H), 0.25-0.04 (br m, 2H) | +* |
| 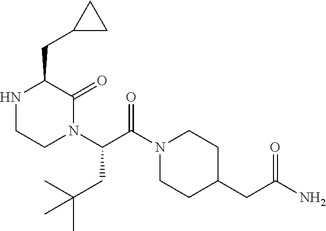 [SMILES: CC(C)(C)C[C@@H](C(=O)N1CCC(CC(N)=O)CC1)N1CCN[C@@H](CC2CC2)C1=O] | 94 | (1-{(S)-2-[(S)-3- (Cyclopropylmethyl)- 2-oxo-1- piperazinyl]-4,4- dimethylvaleryl}-4- piperidyl)acetamide | (4-Piperidyl)acet- amide (Enamine BB) | CD₃OD: δ 5.65 (m, 1H), 4.47 (t, 1H), 2.69 (q, 1H), 0.99-0.75 (br m, 10H), 1.00-0.75 (br m, 4H), 0.57- 0.40 (br m, 2H), 0.24-0.06 (br m, 2H) | ND |
| 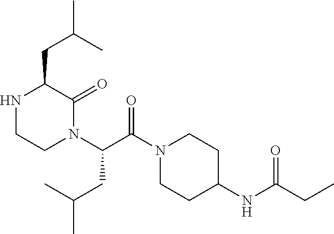 [SMILES: CCC(=O)NC1CCN(C(=O)[C@H](CC(C)C)N2CCN[C@@H](CC(C)C)C2=O)CC1] | 95 | (S)-1-{(S)-3-Methyl- 1-[(4- propionylamino-1- piperidyl)car- bonyl]butyl}-3- isobutyl-2- piperazinone | 1-(4- Piperidylamino)- 1-propanone (Enamine BB) | CD₃OD: δ 5.61 (m, 1H), 2.23 (q, 2H), 1.16 (t, 3H), 1.01 (m, 12H) | +* |
| 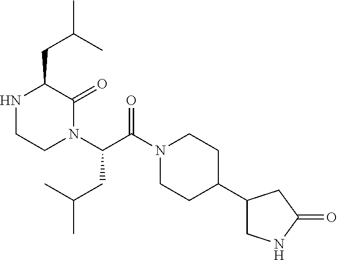 [SMILES: CC(C)C[C@@H]1NCCN([C@@H](CC(C)C)C(=O)N2CCC(C3CNC(=O)C3)CC2)C1=O] | 96 | (S)-1-[(S)-3-Methyl- 1-{[4-(5-oxo-3- pyrrolidinyl)-1- piperidyl]car- bonyl}butyl]-3- isobutyl-2- piperazinone | 4-(4-Piperidyl)-2- pyrrolidinone (Enamine BB) | CD₃OD: δ 5.59 (m, 1H), 4.54 (t, 1H), 2.37 (m, 2H), 1.70 (m, 9H), 1.04 (m, 12H) | ++ |
| 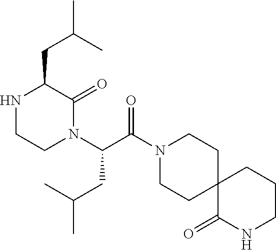 [SMILES: CC(C)C[C@@H]1NCCN([C@@H](CC(C)C)C(=O)N2CCC3(CCCNC3=O)CC2)C1=O] | 97 | (S)-1-[(S)-1-{(2,9- Diazaspiro[5.5]un- decan-9- oyl)carbonyl}-3- methylbutyl]-3- isobutyl-2- piperazinone | 2,9-Diaza-1- spiro[5.5]un- decanone (Combi-Blocks) | CD₃OD: δ 5.67 (m, 1H), 3.18 (m, 2H), 1.82 (m, 16H), 1.03 (m, 12H) | ++ |
| 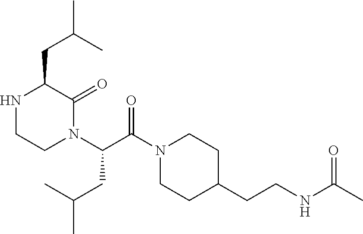 [SMILES: CC(=O)NCCC1CCN(C(=O)[C@H](CC(C)C)N2CCN[C@@H](CC(C)C)C2=O)CC1] | 98 | (S)-1-[(S)-1-{[4-(1- Acetylaminoethyl)- 1- piperidyl]carbonyl}- 3-methylbutyl]-3- isobutyl-2- piperazinone | 1-[2-(4- Piperidyl)ethyl- amino]-1- ethanone (Matrix Scientific) | CD₃OD: δ 5.64 (m, 1H), 4.55 (t, 1H), 2.01 (s, 3H), 1.04 (m, 12H) | +* |
| 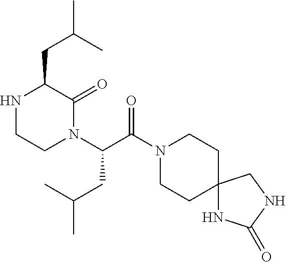 [SMILES: CC(C)C[C@@H]1NCCN([C@@H](CC(C)C)C(=O)N2CCC3(CC2)CNC(=O)N3)C1=O] | 99 | 8-{(S)-2-[(S)-3- Isobutyl-2-oxo-1- piperazinyl]-4- methylvaleryl}- 1,3,8-triaza-2- spiro[4.5]decanone | 1,3,8-Triaza-2- spiro[4.5] decanone (AstaTech) | CD₃OD: δ 5.58 (m, 1H), 2.71 (d, 2H), 1.68 (m, 10H), 0.94 (m, 12H) | +* |
| 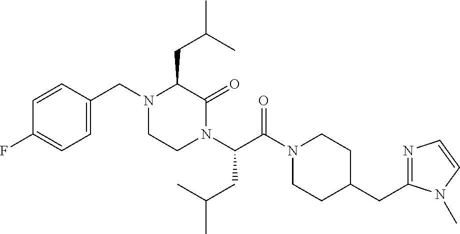 [SMILES: CC(C)C[C@H]1C(=O)N([C@@H](CC(C)C)C(=O)N2CCC(Cc3nccn3C)CC2)CCN1Cc1ccc(F)cc1] | 100 | (S)-1-[(S)-3-Methyl- 1-({4-[(1-methyl- 1H-imidazol-2- yl)methyl]-1- piperidyl}car- bonyl)butyl]-4-[(p- fluorophenyl)meth- yl]-3-isobutyl-2- piperazinone | 1-methyl-2-[(4- piperidyl)methyl]- 1H-imidazole (Enamine BB) | CD₃OD: δ 7.34 (m, 2H) 7.05 (m, 2H), 5.55 (m, 1H), 4.48 (t, 1H), 3.62 (s, 3H), 0.93 (m, 12H) | ++++ |
| 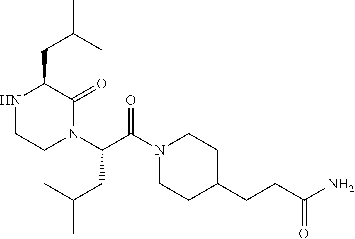 [SMILES: CC(C)C[C@@H]1NCCN([C@@H](CC(C)C)C(=O)N2CCC(CCC(N)=O)CC2)C1=O] | 101 | 3-(1-{(S)-2-[(S)-3- Isobutyl-2-oxo-1- piperazinyl]-4- methylvaleryl}-4- piperidyl)propiona- mide | 3-(4- Piperidyl)pro- pionamide (Enamine BB) | CD₃OD: δ 5.63 (m, 1H), 4.53 (t, 1H), 4.14 (m, 1H), 2.31 (m, 2H), 1.75 (m, 11H), 1.01 (m, 12H) | ++ |
| 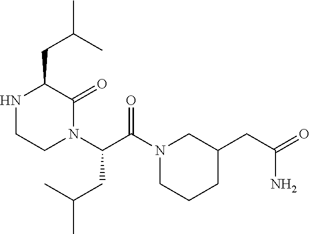 [SMILES: CC(C)C[C@@H]1NCCN([C@@H](CC(C)C)C(=O)N2CCCC(CC(N)=O)C2)C1=O] | 102 | (1-{(S)-2-[(S)-3- Isobutyl-2-oxo-1- piperazinyl]-4- methylvaleryl}-3- piepridyl)acetamide | (3-Piperidyl)acet- amide (Enamine BB) | CD₃OD: δ 5.61 (m, 1H), 2.20 (m, 2H), 1.70 (m, 11H), 0.99 (m, 12H) | +* |
| 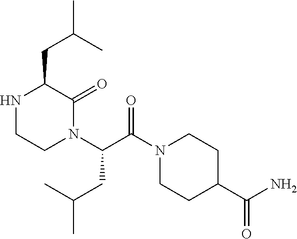 [SMILES: CC(C)C[C@@H]1NCCN([C@@H](CC(C)C)C(=O)N2CCC(C(N)=O)CC2)C1=O] | 103 | 1-{(S)-2-[(S)-3- Isobutyl-2-oxo-1- piperazinyl]-4- methylvaleryl}-4- piperidinecarboxa- mide | 4-Piperidine- carboxamide | CD₃OD: δ 5.64 (m, 1H), 2.59 (m, 1H), 2.10 (m, 1H), 1.74 (m, 10H), 1.01 (m, 12H) | +* |
| 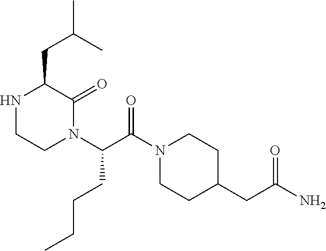 [SMILES: CCCC[C@@H](C(=O)N1CCC(CC(N)=O)CC1)N1CCN[C@@H](CC(C)C)C1=O] | 104 | (1-{(S)-2-[(S)-3- Isobutyl-2-oxo-1- piperazinyl]hexa- noyl}-4- piperidyl)acetamide | (4-Piperidyl)acet- amide (Enamine BB) | CD₃OD: δ 5.43 (m, 1H), 4.47 (t, 1H), 2.69 (q, 1H), 1.01-0.82 (br m, 9H) | +* |
| 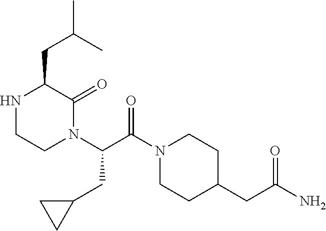 [SMILES: CC(C)C[C@@H]1NCCN([C@@H](CC2CC2)C(=O)N2CCC(CC(N)=O)CC2)C1=O] | 105 | (1-{(S)-2-{(S)-3- Isobutyl-2-oxo-1- piperazinyl]-3- cyclopropylpro- pionyl}-4- piperidyl)acetamide | (4-Piperidyl)acet- amide (Enamine BB) | CD₃OD: δ 5.55 (m, 1H), 4.48 (t, 1H), 2.69 (q, 1H), 0.95 (m, 6H), 0.66 (m, 1H), 0.53-0.35 (br m, 2H), 0.22-0.04 (br m, 2H) | +* |
| 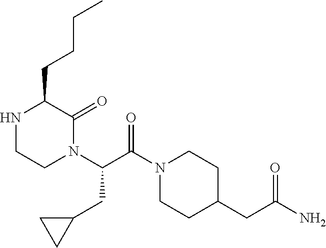 [SMILES: CCCC[C@@H]1NCCN([C@@H](CC2CC2)C(=O)N2CCC(CC(N)=O)CC2)C1=O] | 106 | (1-{(S)-2-[(S)-3- Butyl-2-oxo-1- piperazinyl]-3- cyclopropylpro- pionyl}-4- piperidyl)acetamide | (4-Piperidyl)acet- amide (Enamine BB) | CD₃OD: δ 5.56 (m, 1H), 4.48 (t, 1H), 2.69 (q, 1H), 1.50-1.01 (br m, 6H), 0.93 (m, 3H), 0.66 (m, 1H), 0.56-0.36 (br m, 2H), 0.22- 0.02 (br m, 2H) | ND |
| 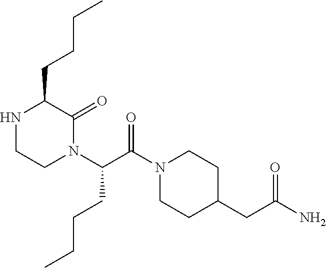 [SMILES: CCCC[C@@H]1NCCN([C@@H](CCCC)C(=O)N2CCC(CC(N)=O)CC2)C1=O] | 107 | (1-{(S)-2-[(S)-3- Butyl-2-oxo-1- piperazinyl]hex- anoyl}-4- piperidyl)acetamide | (4-Piperidyl)acet- amide (Enamine BB) | CD₃OD: δ 5.45 (m, 1H), 4.47 (t, 1H), 2.69 (q, 1H), 1.53-1.01 (br m, 10H), 0.93 (m, 6H) | +* |
| 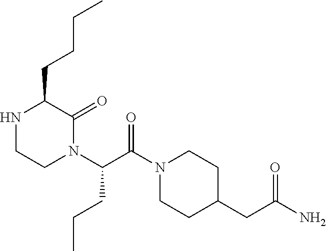 [SMILES: CCCC[C@@H]1NCCN([C@@H](CCC)C(=O)N2CCC(CC(N)=O)CC2)C1=O] | 108 | (1-{(S)-2-[(S)-3- Butyl-2-oxo-1- piperazinyl]valeryl}- 4- piperidyl)acetamide | (4-Piperidyl)acet- amide (Enamine BB) | CD₃OD: δ 5.46 (m, 1H), 4.47 (t, 1H), 2.69 (q, 1H), 1.61-1.01 (br m, 8H), 0.95 (m, 6H) | +* |
| 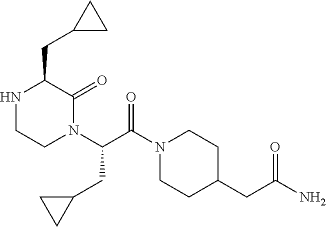 [SMILES: NC(=O)CC1CCN(C(=O)[C@H](CC2CC2)N2CCN[C@@H](CC3CC3)C2=O)CC1] | 109 | (1-{(S)-2-[(S)-3- (Cyclopropylmethyl)- 2-oxo-1- piperazinyl]-3- cyclopropylpro- pionyl}-4- piperidyl)acetamide | (4-Piperidyl)acet- amide (Enamine BB) | CD₃OD: δ 5.56 (m, 1H), 4.49 (t, 1H), 2.69 (q, 1H), 0.85 (m, 1H), 0.66 (m, 1H), 0.57-0.34 (br m, 4H), 0.23-0.01 (br m, 4H) | +* |
| 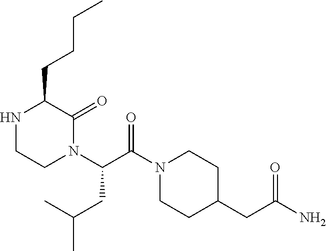 [SMILES: CCCC[C@@H]1NCCN([C@@H](CC(C)C)C(=O)N2CCC(CC(N)=O)CC2)C1=O] | 110 | (1-{(S)-2-[(S)-3- Butyl-2-oxo-1- piperazinyl]-4- methylvaleryl}-4- piperidyl)acetamide | (4-Piperidyl)acet- amide (Enamine BB) | CD₃OD: δ 5.57 (m, 1H), 4.47 (t, 1H), 2.68 (q, 1H), 1.25-1.05 (br m, 2H), 0.95 (m, 9H) | +* |
| 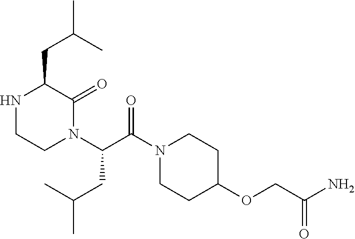 [SMILES: CC(C)C[C@@H]1NCCN([C@@H](CC(C)C)C(=O)N2CCC(OCC(N)=O)CC2)C1=O] | 111 | (1-{(S)-2-[(S)-3- Isobutyl-2-oxo-1- piperazinyl]-4- methylvaleryl}-4- piperidyloxy)aceta- mide | (4-Piperidyl- oxy)acetamide (AstaTech) | CD₃OD: δ 5.65 (m, 1H), 4.07 (s, 2H), 3.51 (m, 1H), 1.77 (m, 10H), 1.03 (m, 12H) | +* |
| 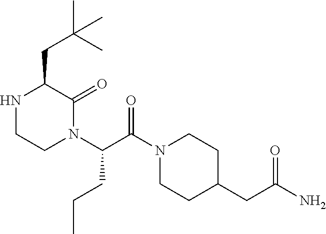 [SMILES: CCC[C@@H](C(=O)N1CCC(CC(N)=O)CC1)N1CCN[C@@H](CC(C)(C)C)C1=O] | 112 | (1-{(S)-2-[(S)-3- Neopentyl-2-oxo- 1- piperazinyl]valeryl}- 4- piperidyl)acetamide | (4-Piperidyl)acet- amide (Enamine BB) | CD₃OD: δ 5.49 (m, 1H), 4.51 (t, 1H), 2.19 (d, 2H), 2.06 (m, 2H), 1.03 (m, 12H) | +* |
| 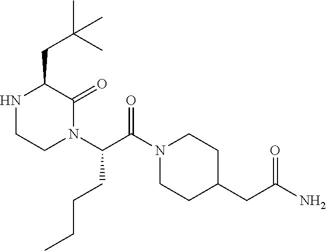 [SMILES: CCCC[C@@H](C(=O)N1CCC(CC(N)=O)CC1)N1CCN[C@@H](CC(C)(C)C)C1=O] | 113 | (1-{(S)-2-[(S)-3- Neopentyl-2-oxo- 1- piperazinyl]hex- anoyl}-4- piperidyl)acetamide | (4-Piperidyl)acet- amide (Enamine BB) | CD₃OD: δ 5.46 (m, 1H), 4.51 (t, 1H), 2.19 (d, 2H), 2.06 (m, 2H), 1.78 (m, 4H), 1.02 (s, 9H), 0.95 (t, 3H) | +* |
| 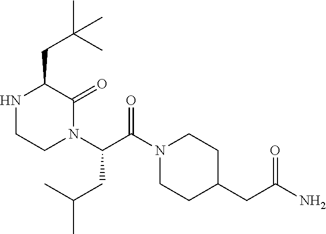 [SMILES: CC(C)C[C@@H](C(=O)N1CCC(CC(N)=O)CC1)N1CCN[C@@H](CC(C)(C)C)C1=O] | 114 | (1-{(S)-2-[(S)-3- Neopentyl-2-oxo- 1-piperazinyl]-4- methylvaleryl}-4- piperidyl)acetamide | (4-Piperidyl)acet- amide (Enamine BB) | CD₃OD: δ 5.60 (m, 1H), 4.51 (t, 1H), 2.19 (d, 2H), 2.06 (m, 2H), 1.03 (s, 9H), 0.99 (m, 6H) | ND |
| 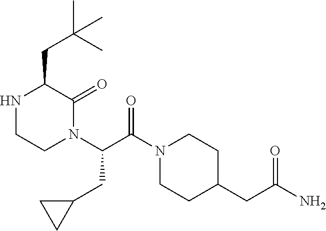 [SMILES: CC(C)(C)C[C@@H]1NCCN([C@@H](CC2CC2)C(=O)N2CCC(CC(N)=O)CC2)C1=O] | 115 | (1-{(S)-2-[(S)-3- Neopentyl-2-oxo- 1-piperazinyl]-3- cyclopropylpro- pionyl}-4- piperidyl)acetamide | (4-Piperidyl)acet- amide (Enamine BB) | CD₃OD: δ 5.58 (m, 1H), 4.52 (t, 1H), 2.20 (d, 2H), 1.03 (s, 9H), 0.69 (m, 1H), 0.49 (m, 2H), 0.16 (m, 2H) | ++ |
| 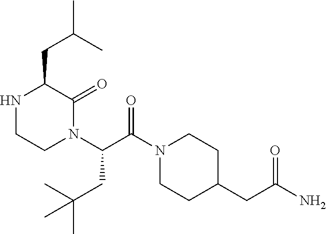 [SMILES: CC(C)C[C@@H]1NCCN([C@@H](CC(C)(C)C)C(=O)N2CCC(CC(N)=O)CC2)C1=O] | 116 | (1-{(S)-2-[(S)-3- Isobutyl-2-oxo-1- piperazinyl]-4,4- dimethylvaleryl}-4- piperidyl)acetamide | (4-Piperidyl)acet- amide (Enamine BB) | CD₃OD: δ 5.68 (m, 1H), 4.51 (t, 1H), 2.08 (m, 2H), 1.82 (m, 4H), 0.97 (m, 15H) | +* |
| 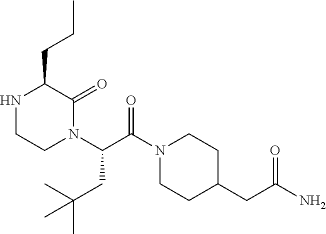 [SMILES: CCC[C@@H]1NCCN([C@@H](CC(C)(C)C)C(=O)N2CCC(CC(N)=O)CC2)C1=O] | 117 | (1-{(S)-2-[(S)-2- Oxo-3-propyl-1- piperazinyl]-4,4- dimethylvaleryl}-4- piperidyl)acetamide | (4-Piperidyl)acet- amide (Enamine BB) | CD₃OD: δ 5.70 (m, 1H), 4.52 (t, 1H), 1.85 (m, 3H), 1.69 (m, 1H), 0.98 (m, 12H) | +* |
| 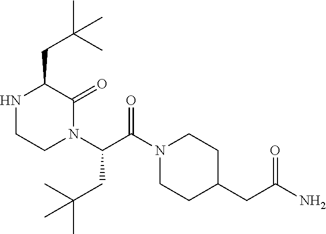 [SMILES: CC(C)(C)C[C@@H]1NCCN([C@@H](CC(C)(C)C)C(=O)N2CCC(CC(N)=O)CC2)C1=O] | 118 | (1-{(S)-2-[(S)-3- Neopentyl-2-oxo- 1-piperazinyl]-4,4- dimethylvaleryl}-4- piperidyl)acetamide | (4-Piperidyl)acet- amide (Enamine BB) | CD₃OD: δ 5.68 (m, 1H), 4.50 (t, 1H), 2.18 (d, 2H), 2.05 (m, 3H), 1.81 (m, 2H), 1.02 (s, 9H), 0.96 (d, 9H) | +* |
| 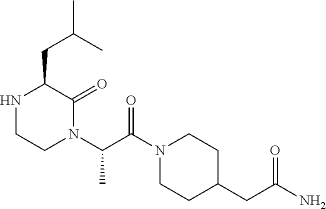 [SMILES: CC(C)C[C@@H]1NCCN([C@@H](C)C(=O)N2CCC(CC(N)=O)CC2)C1=O] | 119 | (1-{(S)-2-[(S)-3- Isobutyl-2-oxo-1- piperazinyl]pro- pionyl}-4- piperidyl)acetamide | (4-Piperidyl)acet- amide (Enamine BB) | CD₃OD: δ 5.51 (m, 1H), 4.52 (t, 1H), 2.20 (d, 2H), 1.34 (d, 3H), 0.99 (t, 6H) | +* |
| 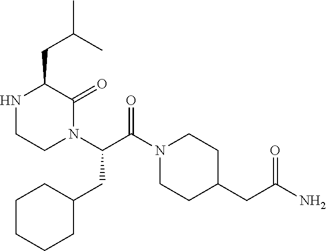 [SMILES: CC(C)C[C@@H]1NCCN([C@@H](CC2CCCCC2)C(=O)N2CCC(CC(N)=O)CC2)C1=O] | 120 | (1-{(S)-2-[(S)-3- Isobutyl-2-oxo-1- piperazinyl]-3- cyclohexylpro- pionyl}-4- piperidyl)acetamide | (4-Piperidyl)acet- amide (Enamine BB) | CD₃OD: δ 5.64 (m, 1H), 4.52 (t, 1H), 3.48 (dd, 1H), 2.19 (d, 2H), 1.80 (m, 10H), 1.00 (s, 6H) | ++ |
| 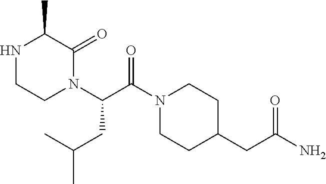 [SMILES: CC(C)C[C@@H](C(=O)N1CCC(CC(N)=O)CC1)N1CCN[C@@H](C)C1=O] | 121 | (1-{(S)-2-[(S)-3- Methyl-2-oxo-1- piperazinyl]-4- methylvaleryl}-4- piperidyl)acetamide | (4-Piperidyl)acet- amide (Enamine BB) | CD₃OD: δ 5.63 (m, 1H), 4.53 (t, 1H), 3.56 (q, 1H), 2.21 (d, 2H), 1.42 (d, 3H), 1.02 (m, 6H) | +* |
| 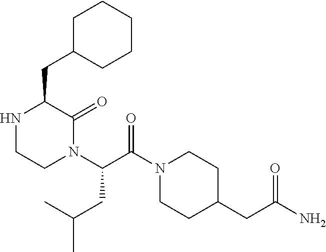 [SMILES: CC(C)C[C@@H](C(=O)N1CCC(CC(N)=O)CC1)N1CCN[C@@H](CC2CCCCC2)C1=O] | 122 | (1-{(S)-2-[(S)-3- (Cyclohexylmethyl)- 2-oxo-1- piperazinyl]-4- methylvaleryl}-4- piperidyl)acetamide | (4-Piperidyl)acet- amide (Enamine BB) | CD₃OD: δ 5.59 (m, 1H), 4.50 (t, 1H), 3.50 (dd, 1H), 2.18 (d, 2H), 1.77 (m, 10H), 0.99 (m, 6H) | ++ |
| 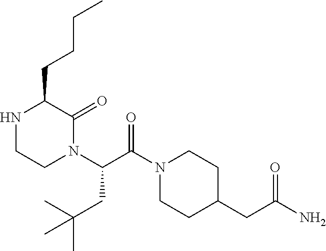 [SMILES: CCCC[C@@H]1NCCN([C@@H](CC(C)(C)C)C(=O)N2CCC(CC(N)=O)CC2)C1=O] | 123 | (1-{(S)-2-[(S)-3- Butyl-2-oxo-1- piperazinyl]-4,4- dimethylvaleryl}-4- piperidyl)acetamide | (4-Piperidyl)acet- amide (Enamine BB) | CD₃OD: δ 5.65 (m, 1H), 4.47 (t, 1H), 2.69 (q, 1H), 1.22-1.00 (br m, 2H), 0.93 (m, 12H) | +* |
| 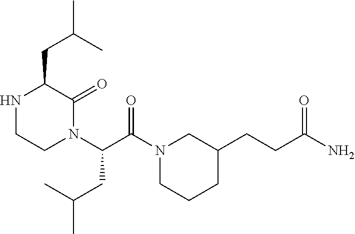 [SMILES: CC(C)C[C@@H]1NCCN([C@@H](CC(C)C)C(=O)N2CCCC(CCC(N)=O)C2)C1=O] | 124 | 3-(1-{(S)-2-[(S)-3- Isobutyl-2-oxo-1- piperazinyl]-4- methylvaleryl}-3- piperidyl)propiona- mide | 3-(3- Piperidyl)pro- pionamide (Enamine BB) | CD₃OD: δ 5.56 (m, 1H), 4.43- 4.04 (br m, 1H), 2.34-2.16 (br m, 2H), 0.95 (m, 12H) | +* |
| 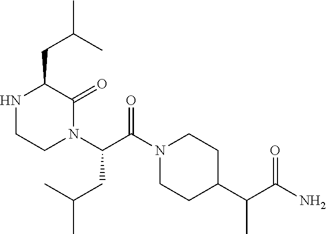 [SMILES: CC(C)C[C@@H]1NCCN([C@@H](CC(C)C)C(=O)N2CCC(C(C)C(N)=O)CC2)C1=O] | 125 | 2-(1-{(S)-2-[(S)-3- Isobutyl-2-oxo-1- piperazinyl]-4- methylvaleryl}-4- piperidyl)propiona- mide | 2-(1-tert- Butoxycarbonyl- 4-piperidyl)pro- pionic acid (AstaTech); amide coupling with NH₄OH, BOC removal | CD₃OD: δ 5.55 (m, 1H), 4.52 (m, 1H), 2.13 (m, 1H), 1.12 (d, 3H), 0.95 (m, 12H) | +* |
| 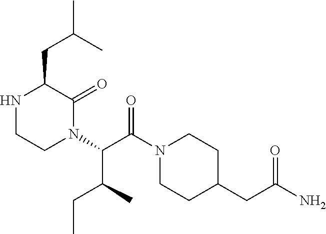 [SMILES: CC[C@H](C)[C@@H](C(=O)N1CCC(CC(N)=O)CC1)N1CCN[C@@H](CC(C)C)C1=O] | 126 | (1-{(2S,3S)-2-[(S)-3- Isobutyl-2-oxo-1- piperazinyl]-3- methylvaleryl}-4- piperidyl)acetamide | (4-Piperidyl)acet- amide (Enamine BB) | CD₃OD: δ 5.25 (dd, 1H), 4.56 (t, 1H), 2.19 (d, 2H), 1.84 (m, 4H), 0.97 (m, 12H) | +* |
| 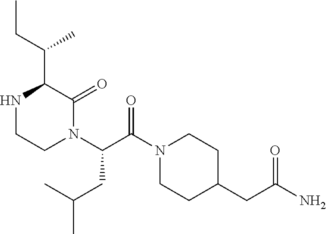 [SMILES: CC[C@H](C)[C@@H]1NCCN([C@@H](CC(C)C)C(=O)N2CCC(CC(N)=O)CC2)C1=O] | 127 | {1-[(S)-2-{(S)-3-[(S)- 1-Methylpropyl]-2- oxo-3,4,5,6- tetrahydro-1H- pyrazin-1-yl}-4- methylvaleryl]-4- piepridyl}acetamide | (4-Piperidyl)acet- amide (Enamine BB) | CD₃OD: δ 5.57 (m, 1H), 4.47 (t, 1H), 2.14 (d, 2H), 1.78 (m, 3H), 1.51 (m, 2H), 0.96 (m, 12H) | +* |
| 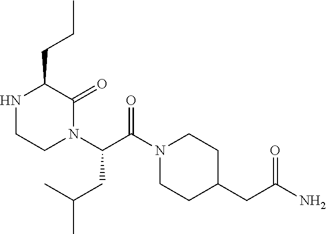 [SMILES: CCC[C@@H]1NCCN([C@@H](CC(C)C)C(=O)N2CCC(CC(N)=O)CC2)C1=O] | 128 | (1-{(S)-2-[(S)-2- Oxo-3-propyl-1- piperazinyl]-4- methylvaleryl}-4- piperidyl)acetamide | (4-Piperidyl)acet- amide (Enamine BB) | CD₃OD: δ 5.60 (m, 1H), 4.50 (t, 1H), 3.14 (m, 2H), 2.17 (d, 2H), 0.99 (m, 9H) | +* |
| 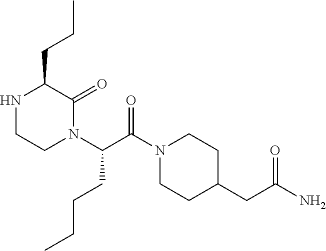 [SMILES: CCCC[C@@H](C(=O)N1CCC(CC(N)=O)CC1)N1CCN[C@@H](CCC)C1=O] | 129 | (1-{(S)-2-[(S)-2- Oxo-3-propyl-1- piperazinyl]hex- anoyl}-4- piperidyl)acetamide | (4-Piperidyl)acet- amide (Enamine BB) | CD₃OD: δ 5.48 (m, 1H), 4.50 (t, 1H), 3.44 (dd, 2H), 2.17 (d, 2H), 1.45 (m, 4H), 0.97 (m, 9H) | +* |
| 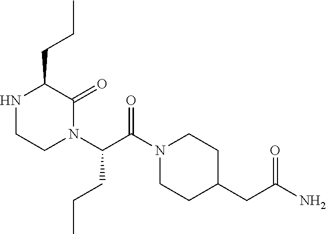 [SMILES: CCC[C@@H]1NCCN([C@@H](CCC)C(=O)N2CCC(CC(N)=O)CC2)C1=O] | 130 | (1-{(S)-2-[(S)-2- Oxo-3-propyl-1- piperazinyl]valeryl}- 4- piperidyl)acetamide | (4-Piperidyl)acet- amide (Enamine BB) | CD₃OD: δ 5.50 (m, 1H), 4.51 (t, 1H), 3.12 (m, 2H), 2.18 (d, 2H), 1.78 (m, 6H), 1.48 (m, 2H), 0.99 (t, 6H) | +* |
| 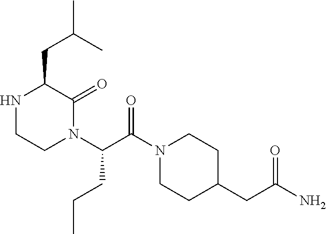 [SMILES: CCC[C@@H](C(=O)N1CCC(CC(N)=O)CC1)N1CCN[C@@H](CC(C)C)C1=O] | 131 | (1-{(S)-2-[(S)-3- Isobutyl-2-oxo-1- piperazinyl]valeryl}- 4- piperidyl)acetamide | (4-Piperidyl)acet- amide (Enamine BB) | CD₃OD: δ 5.49 (m, 1H), 4.51 (t, 1H), 3.13 (m, 2H), 2.18 (d, 2H), 1.79 (m, 6H), 0.98 (m, 9H) | +* |
| 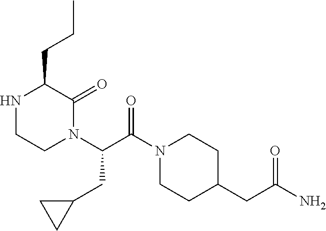 [SMILES: CCC[C@@H]1NCCN([C@@H](CC2CC2)C(=O)N2CCC(CC(N)=O)CC2)C1=O] | 132 | (1-{(S)-2-[(S)-2- Oxo-3-propyl-1- piperazinyl]-3- cyclopropylpro- pionyl}-4- piperidyl)acetamide | (4-Piperidyl)acet- amide (Enamine BB) | CD₃OD: δ 5.59 (m, 1H), 4.54 (t, 1H), 2.19 (d, 2H), 0.99 (t, 3H), 0.69 (m, 1H), 0.49 (m, 2H), 0.16 (m, 2H) | +* |
| 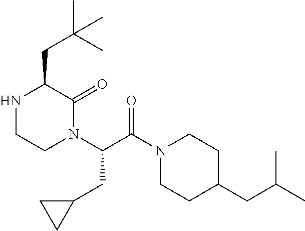 [SMILES: CC(C)CC1CCN(C(=O)[C@H](CC2CC2)N2CCN[C@@H](CC(C)(C)C)C2=O)CC1] | 133 | (S)-1-[(S)-1- (Cyclopropylmethyl)- 2-(4-isobutyl-1- piperidyl)-2- oxoethyl]-3- neopentyl-2- piperazinone | 4- Isobutylpiperidine (Enamine BB) | CD₃OD: δ 5.58 (q, 1H), 4.54 (t, 1H), 0.96 (m, 9H), 0.85 (d, 6H), 0.63 (m, 1H), 0.42 (m, 2H), 0.08 (m, 2H) | +++ |
| 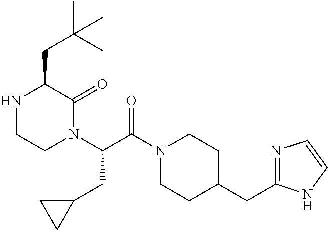 [SMILES: CC(C)(C)C[C@@H]1NCCN([C@@H](CC2CC2)C(=O)N2CCC(Cc3ncc[nH]3)CC2)C1=O] | 134 | (S)-1-[(S)-1- (Cyclopropylmethyl)- 2-{4-[(1H- imidazol-2- yl)methyl]-1- piperidyl}-2- oxoethyl]-3- neopentyl-2- piperazinone | 2-[(4- Piperidyl)methyl]- 1H-imidazole (Enamine BB) | CD₃OD: δ 6.91 (s, 2H), 5.51 (m, 1H), 4.45 (t, 1H), 0.96 (m, 9H), 0.62 (m, 1H), 0.41 (m, 2H), 0.08 (m, 2H) | +* |
| 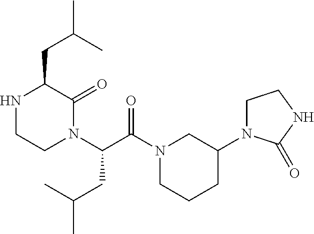 [SMILES: CC(C)C[C@@H]1NCCN([C@@H](CC(C)C)C(=O)N2CCCC(N3CCNC3=O)C2)C1=O] | 135 | 1-(1-{(S)-2-[(S)-3- Isobutyl-2-oxo-1- piperazinyl]-4- methylvaleryl}-3- piperidyl)-2- imidazolidinone | 1-(3-Piperidyl)-2- imidazolidinone (Enamine BB) | CD₃OD: δ 5.59 (m, 1H), 4.53 (t, 1H), 2.99 (m, 1H), 1.71 (m, 10H), 0.99 (m, 12H) | +* |
| 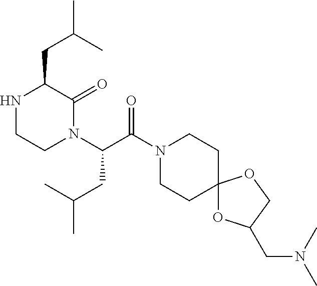 [SMILES: CC(C)C[C@@H]1NCCN([C@@H](CC(C)C)C(=O)N2CCC3(CC2)OCC(CN(C)C)O3)C1=O] | 232 | (S)-1-[(S)-1-({2- [(Dimethyl- amino)methyl]- 1,4-dioxa-8-aza-8- spiro[4.5]decyl}car- bonyl)-3- methylbutyl]-3- isobutyl-2- piperazinone | Benzyl 4-oxo-1- piperidinecarbox- ylate, ketalization with Glycerol, mesylation of alcohol, substitution of mesylate using Dimethylamine, Cbz removal | CD₃OD: δ 5.59, (dd, 1H), 4.37 (m, 1H), 2.39 (s, 6H), 0.97 (m, 12H | ++++ |
| 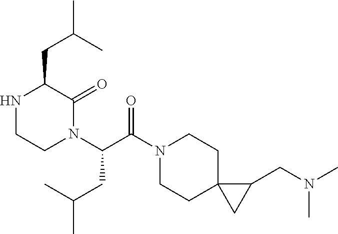 [SMILES: CC(C)C[C@@H]1NCCN([C@@H](CC(C)C)C(=O)N2CCC3(CC2)CC3CN(C)C)C1=O] | 233 | (S)-1-[(S)-1-({1- [(Dimethyl- amino)methyl]-6- aza-6- spiro[2.5]octyl}car- bonyl)-3- methylbutyl]-3- isobutyl-2- piperazinone | tert-Butyl 1- (hydroxymethyl)- 6-aza-6- spiro[2.5]octane carboxylate (Enamine BB), mesylation of alcohol, substitution of mesylate using Dimethylamine, BOC removal | CD₃OD: δ 5.63-5.55, (m, 1H), 2.30 (s, 6H), 1.00-0.93 (m, 12H), 0.28 (m, 1H) | +++ |
| 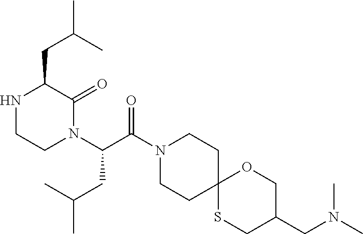 [SMILES: CC(C)C[C@@H]1NCCN([C@@H](CC(C)C)C(=O)N2CCC3(CC2)OCC(CN(C)C)CS3)C1=O] | 234 | (S)-1-[(S)-1-({3- [(Dimethyl- amino)methyl]- 1-oxa-5- thia-9-aza-9- spiro[5.5]un- decyl}car- bonyl)-3- methylbutyl]-3- isobutyl-2- piperazinone | (2,2-Dimethyl- 1,3-dioxan-5- yl)methanol (Combi-Blocks), mesylation of alcohol, substitution with potassium thioacetate, reduction of thioacetate to thiol, ketalization with Benzyl 4-oxo-1- iperidinecarboxyl- | CD₃OD: δ 5.58 (dd, 1H), 2.82- 2.72 (m, 1H), 2.53-2.43 (m, 1H), 0.97 (m, 12H) | ++++ |
| | | | ate, mesylation | | |
| | | | of alcohol, | | |
| | | | substitution of | | |
| | | | mesylate using | | |
| | | | Dimethylamine, | | |
| | | | Cbz removal | | |
| 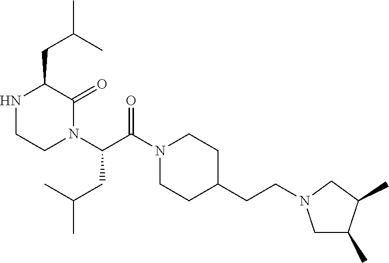 [SMILES: CC(C)C[C@@H]1NCCN([C@@H](CC(C)C)C(=O)N2CCC(CCN3C[C@@H](C)[C@@H](C)C3)CC2)C1=O] | 235 | (S)-1-[(S)-1-[(4-{2- [(3R,4S)-3,4- Dimethyl-1- pyrrolidinyl]ethyl}- 1- piperidyl)carbonyl]- 3-methylbutyl]-3- isobutyl-2- piperazinone | tert-Butyl 4- (formylmethyl)- 1- piperidinecarbox- ylate, reductive amination with (3R,4S)-3,4- Dimethylpyrroli- dine (ChemBridge BB), BOC removal | CD₃OD: δ 5.55 (m, 1H), 4.50 (m, 1H), 2.75 (2s, 6H), 0.96 (m, 12H) | ++++ |
| 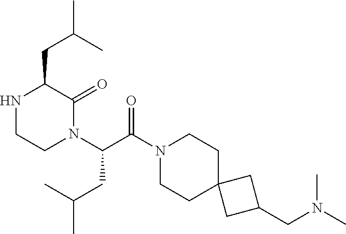 [SMILES: CC(C)C[C@@H]1NCCN([C@@H](CC(C)C)C(=O)N2CCC3(CC2)CC(CN(C)C)C3)C1=O] | 236 | (S)-1-[(S)-1-({2- [(Dimethyl- amino)methyl]- 7-aza-7- spiro[3.5]nonyl}car- bonyl)-3- methylbutyl]-3- isobutyl-2- piperazinone | tert-Butyl 2-oxo- 7-aza-7- spiro[3.5]nonane carboxylate (AstaTech), Wittig reaction with Methyltriphenyl phosphonium bromide, hydroboration, oxidation to aldehyde, reductive amination with | CD₃OD: δ 5.56 (m, 1H), 2.96 (m, 1H), 2.24 (s, 6H), 0.95 (m, 12H) | *+ |
| | | | Dimethylamine, | | |
| | | | BOC removal | | |
| 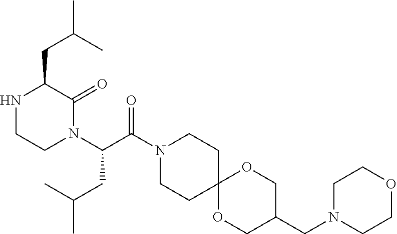 [SMILES: CC(C)C[C@@H]1NCCN([C@@H](CC(C)C)C(=O)N2CCC3(CC2)OCC(CN2CCOCC2)CO3)C1=O] | 237 | (S)-1-[(S)-3-Methyl- 1-({3- (morpholinomethyl)- 1,5-dioxa-9-aza- 9- spiro[5.5]un- decyl}car- bonyl)butyl]-3- isobutyl-2- piperazinone | Benzyl 4-oxo-1- piperidinecarbox- ylate, ketalization with (2,2-Dimethyl- 1,3-dioxan-5- yl)methanol (Combi-Blocks), tosylation of alcohol, substitution of tosylate using Morpholine, Cbz removal | CDCl₃: δ 5.56 (t, 1H), 2.34 (br s, 4H), 2.26 (t, 2H), 0.90 (m, 12H) | ++++ |
| 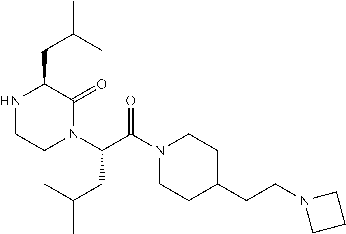 [SMILES: CC(C)C[C@@H]1NCCN([C@@H](CC(C)C)C(=O)N2CCC(CCN3CCC3)CC2)C1=O] | 238 | (S)-1-[(S)-1-({4-[2- (1- Azetidinyl)ethyl]-1- piperidyl}carbonyl)- 3-methylbutyl]-3- isobutyl-2- piperazinone | tert-Butyl 4- (formylmethyl)- 1- piperidinecarbox- ylate, reductive amination with Azetidine, BOC removal | CD₃OD: δ 5.56 (m, 1H), 4.47 (m, 1H), 4.08 (m, 1H), 2.63 (m, 3H), 2.16 (m, 2H), 1.66 (m, 10H), 1.32 (m, 2H), 1.03 (m, 15H) | ++++ |
| 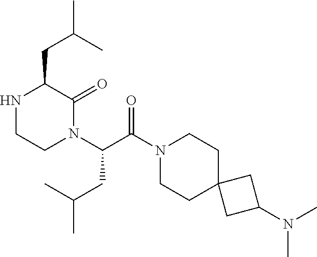 [SMILES: CC(C)C[C@@H]1NCCN([C@@H](CC(C)C)C(=O)N2CCC3(CC2)CC(N(C)C)C3)C1=O] | 239 | (S)-1-[(S)-1-({2- (Dimethylamino)- 7-aza-7- spiro[3.5]nonyl}car- bonyl)-3- methylbutyl]-3- isobutyl-2- piperazinone | tert-Butyl 2-oxo- 7-aza-7- spiro[3.5]nonane carboxylate (AstaTech), reductive amination with Dimethylamine, BOC removal | CD₃OD: δ 5.56 (m, 1H), 2.91 (m, 1H), 2.72 (m, 1H), 2.12 (m, 8H), 1.65 (m, 13H), 0.96 (m, 12H) | *+ |
| 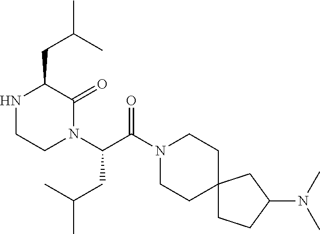 [SMILES: CC(C)C[C@@H]1NCCN([C@@H](CC(C)C)C(=O)N2CCC3(CCC(N(C)C)C3)CC2)C1=O] | 240 | (S)-1-[(S)-1-({2- (Dimethylamino)- 8-aza-8- spiro[4.5]decyl}car- bonyl)-3- methylbutyl]-3- isobutyl-2- piperazinone | tert-Butyl 2-oxo- 8- azaspiro[4.5]dec- ane-8- carboxylate (AstaTech), reductive amination with Dimethylamine, BOC removal | CD₃OD: δ 5.56 (m, 1H), 3.67 (m, 2H), 2.91 (m, 1H), 2.71 (m, 1H), 2.31 (s, 6H), 1.63 (m, 17H), 0.97 (m, 12H) | ++++ |
| 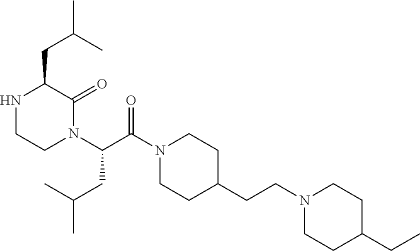 [SMILES: CCC1CCN(CCC2CCN(C(=O)[C@H](CC(C)C)N3CCN[C@@H](CC(C)C)C3=O)CC2)CC1] | 241 | (S)-1-[(S)-1-({4-[2- (4-Ethyl-1- piperidyl)ethyl]-1- piperidyl}carbonyl)- 3-methylbutyl]-3- isobutyl-2- piperazinone | tert-Butyl 4- (formylmethyl)- 1- piperidinecarbox- ylate, reductive amination with 4-Ethylpiperidine (AstaTech), BOC removal | CD₃OD: δ 5.55 (m, 1H), 4.47 (m, 1H), 4.06 (m, 1H), 2.92 (m, 7H), 1.98 (m, 2H), 1.65 (m, 14H), 1.12 (m, 24H) | ++++ |
| 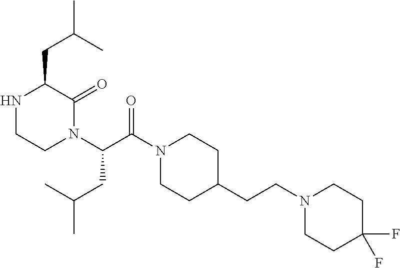 [SMILES: CC(C)C[C@@H]1NCCN([C@@H](CC(C)C)C(=O)N2CCC(CCN3CCC(F)(F)CC3)CC2)C1=O] | 242 | (S)-1-[(S)-1-({4-[2- (4,4-Difluoro-1- piperidyl)ethyl]-1- piperidyl}carbonyl)- 3-methylbutyl]-3- isobutyl-2- piperazinone | tert-Butyl 4- (formylmethyl)- 1- piperidinecarbox- ylate, reductive amination with 4,4- Difluoropiperidine (AstaTech), BOC removal | CD₃OD: δ 5.56 (m, 1H), 4.47 (m, 1H), 4.08 (m, 2H), 2.57 (m, 6H), 1.75 (m, 15H), 1.05 (m, 15H) | ++++ |
| 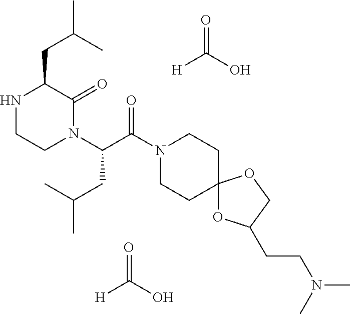 [SMILES: CC(C)C[C@@H]1NCCN([C@@H](CC(C)C)C(=O)N2CCC3(CC2)OCC(CCN(C)C)O3)C1=O ; O=CO ; O=CO] | 243 | (S)-1-[(S)-1-({2-[2- (Dimethylamino)eth- yl]-1,4-dioxa-8- aza-8- spiro[4.5]decyl}car- bonyl)-3- methylbutyl]-3- isobutyl-2- piperazinone, bis formic acid salt | Benzyl 4-oxo-1- piperidinecarbox- ylate, ketalization with 1,2,4-Butanetriol (Combi-Blocks), tosylation of alcohol, substitution of tosylate using Dimethylamine, Cbz removal, Formic acid treatment | CD₃OD: δ 5.56 (m, 1H), 2.89 (s, 6H), 2.12- 1.35 (br m, 12H), 1.07- 0.85 (br m, 12H) | ++++ |
| 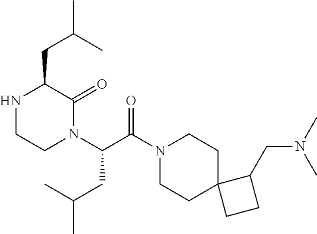 [SMILES: CC(C)C[C@@H]1NCCN([C@@H](CC(C)C)C(=O)N2CCC3(CCC3CN(C)C)CC2)C1=O] | 244 | (S)-1-[(S)-1-({1- [(Dimethyl- amino)methyl]- 7-aza-7- spiro[3.5]nonyl}car- bonyl)-3- methylbutyl]-3- isobutyl-2- piperazinone | tert-Butyl 1-oxo- 7-aza-7- spiro[3.5]nonane carboxylate (AstaTech), Wittig reaction with Methyltriphenyl phosphonium bromide, hydroboration, oxidation to aldehyde, reductive amination with | CD₃OD: δ 5.57 (m, 1H), 3.14 (m, 2H), 3.02- 2.64 (br m, 2H), 2.48 (m, 1H), 0.96 (m, 12H) | ++++ |
| | | | Dimethylamine, | | |
| | | | BOC removal | | |
| 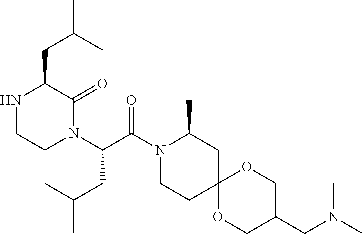 [SMILES: CC(C)C[C@@H]1NCCN([C@@H](CC(C)C)C(=O)N2CCC3(C[C@@H]2C)OCC(CN(C)C)CO3)C1=O] | 245 | (S)-1-[(S)-1-({(S)-3- [(Dimethyl- amino)methyl]- 8-methyl- 1,5-dioxa-9-aza-9- spiro[5.5]un- decyl}car- bonyl)-3- methylbutyl]-3- isobutyl-2- piperazinone | (S)-1-[(S)-1- Phenylethyl]-2- methyl-4- piperidinone (AstaTech), ketalization with (2,2-Dimethyl- 1,3-dioxan-5- yl)methanol (Combi-Blocks), tosylation of alcohol, substitution of tosylate with Dimethylamine, | CD₃OD: δ 5.54 (m, 1H), 3.68 (m, 2H), 3.11 (m, 1H), 2.52- 2.16 (br m, 9H), 0.95 (m, 12H) | ++++ |
| | | | catalytic | | |
| | | | hydrogenation | | |
| 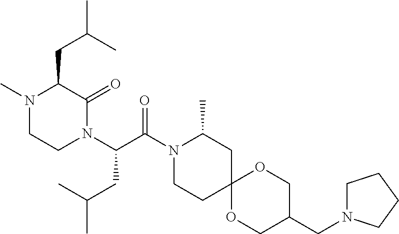 [SMILES: CC(C)C[C@H]1C(=O)N([C@@H](CC(C)C)C(=O)N2CCC3(C[C@H]2C)OCC(CN2CCCC2)CO3)CCN1C] | 246 | (S)-1-[(S)-1-({(R)-8- Methyl-3-[(1- pyrrolidinyl)methyl]- 1,5-dioxa-9-aza- 9- spiro[5.5]un- decyl}carbonyl)-3- methylbutyl]-3- isobutyl-4-methyl- 2-piperazinone | (R)-1-[(S)-1- Phenylethyl)-2- methyl-4- piperidinone (AstaTech), ketalization with (2,2-Dimethyl- 1,3-dioxan-5- yl)methanol (Combi-Blocks), tosylation of alcohol, substitution of tosylate with Pyrrolidine, | CDCl₃: δ 5.56 (m, 1H), 3.07- 2.76 (br m, 2H), 2.33 (s, 3H), 1.09-0.76 (br m, 12H) | ++++ |
| | | | catalytic | | |
| | | | hydrogenation | | |
| 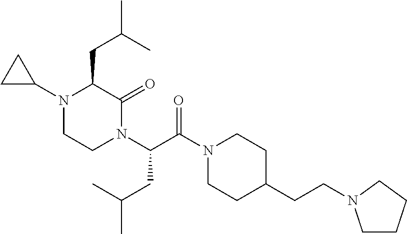 [SMILES: CC(C)C[C@@H](C(=O)N1CCC(CCN2CCCC2)CC1)N1CCN(C2CC2)[C@@H](CC(C)C)C1=O] | 247 | (S)-1-[(S)-3-Methyl- 1-({4-[2-(1- pyrrolidineyl)ethyl]- 1- piperidyl}car- bonyl)butyl]-4- cyclopropyl-3- isobutyl-2- piperazinone | tert-Butyl 4- (formylmethyl)- 1- piperidinecarbox- ylate, reductive amination with Pyrrolidine, BOC removal | CD₃OD: δ 5.58 (m, 1H), 4.47 (m, 1H), 3.58 (m, 1H), 0.94 (m, 12H), 0.62- 0.38 (br m, 4H | ++++ |
| 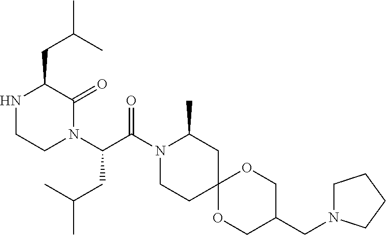 [SMILES: CC(C)C[C@@H]1NCCN([C@@H](CC(C)C)C(=O)N2CCC3(C[C@@H]2C)OCC(CN2CCCC2)CO3)C1=O] | 248 | (S)-1-[(S)-1-({(S)-8- Methyl-3-[(1- pyrrolidinyl)methyl]- 1,5-dioxa-9-aza- 9- spiro[5.5]un- decyl}carbonyl)-3- methylbutyl]-3- isobutyl-2- piperazinone | (S)-1-[(S)-1- Phenylethyl]-2- methyl-4- piperidinone (AstaTech), ketalization with (2,2-Dimethyl- 1,3-dioxan-5- yl)methanol (Combi-Blocks), tosylation of alcohol, substitution of tosylate with Pyrrolidine, | CD₃OD: δ 5.54 (m, 1H), 4.13- 3.90 (m, 2H), 3.78-3.61 (m, 2H), 2.74-2.55 (m, 5H), 0.95 (m, 12H) | ++++ |
| | | | catalytic | | |
| | | | hydrogenation | | |
| 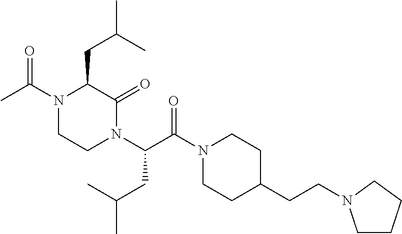 [SMILES: CC(=O)N1CCN([C@@H](CC(C)C)C(=O)N2CCC(CCN3CCCC3)CC2)C(=O)[C@@H]1CC(C)C] | 249 | (S)-1-[(S)-3-Methyl- 1-({4-[2-(1- pyrrolidinyl)ethyl]- 1- piperidyl}car- bonyl)butyl]-4- acetyl-3- isobutyl-2- piperazinone | tert-Butyl 4- (formylmethyl)- 1- piperidinecarbox- ylate, reductive amination with Pyrrolidine, BOC removal | CD₃OD: δ 5.51 (m, 1H), 1.52 (m, 4H), 2.28 (s, 3H), 0.88 (m, 12H) | ++++ |
| 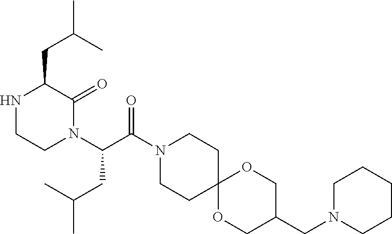 [SMILES: CC(C)C[C@@H]1NCCN([C@@H](CC(C)C)C(=O)N2CCC3(CC2)OCC(CN2CCCCC2)CO3)C1=O] | 250 | (S)-1-[(S)-3-Methyl- 1-({3- (piperidinomethyl)- 1,5-dioxa-9-aza-9- spiro[5.5]un- decyl}car- bonyl)butyl]-3- isobutyl-2- piperazinone | Benzyl 4-oxo-1- piperidinecarbox- ylate, ketalization with (2,2-Dimethyl- 1,3-dioxan- yl)methanol (Combi-Blocks), tosylation of alcohol, substitution of tosylate using Piperidine, Cbz removal | CD₃OD: δ 5.61 (t, 1H), 3.16 (m, 1H), 2.23 (t, 2H), 1.59- 1.35 (m, 10H), 0.97 (m, 12H) | ++++ |
| 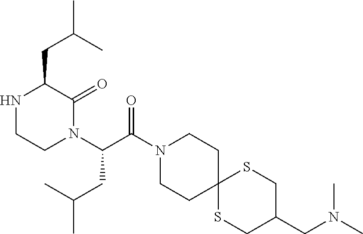 [SMILES: CC(C)C[C@@H]1NCCN([C@@H](CC(C)C)C(=O)N2CCC3(CC2)SCC(CN(C)C)CS3)C1=O] | 251 | (S)-1-[(S)-1-({3- [(Dimethyl- amino)methyl]- 1,5-dithia- 9-aza-9- spiro[5.5]un- decyl}car- bonyl)-3- methylbutyl]-3- isobutyl-2- piperazinone | 3-Bromo-2- (bromo- methyl)propionic acid (AstaTech), substitution with potassium thioacetate, esterification of carboxylic acid, reduction with LAH, thio- ketalization with Benzyl 4-oxo-1- piperidinecarbox- ylate, mesylation | CD₃OD: δ 5.58 (t, 1H), 2.62 (m, 2H), 2.21 (s, 6H), 1.72- 1.36 (m, 6H) 0.97 (m, 12H) | ++++ |
| | | | of alcohol, | | |
| | | | substitution of | | |
| | | | mesylate using | | |
| | | | Dimethylamine, | | |
| | | | Cbz removal | | |
| 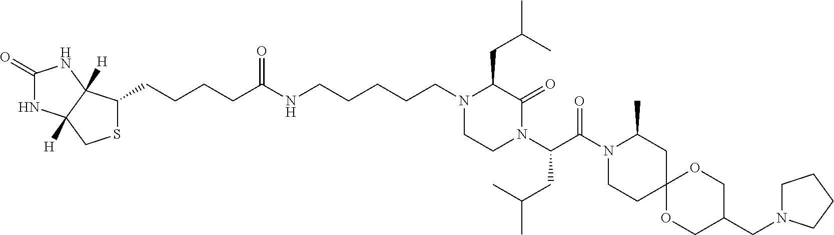 [SMILES: CC(C)C[C@H]1C(=O)N([C@@H](CC(C)C)C(=O)N2CCC3(C[C@@H]2C)OCC(CN2CCCC2)CO3)CCN1CCCCCNC(=O)CCCC[C@@H]1SC[C@@H]2NC(=O)N[C@@H]21] | 252 | (1R,5S,6S)-6-(5-{5- [(S)-4-[(S)-1-({(S)-8- Methyl-3-[(1- pyrrolidinyl)methyl]- 1,5-dioxa-9-aza- 9- spiro[5.5]un- decyl}car- bonyl)-3- methylbutyl]-2- isobutyl-3-oxo-1- piperazinyl]pentyla- mino}-5- oxopentyl)-7-thia- 2.4-diaza- bicyclo[3.3.0] | (S)-1-[(S)-1- Phenylethyl]-2- methyl-4- piperidinone (AstaTech), ketalization with (2,2-Dimethyl- 1,3-dioxan-5- yl)methanol (Combi-Blocks), tosylation of alcohol, substitution of tosylate with Pyrrolidine, catalytic | CD₃OD: δ 5.54 (m, 1H), 4.51 (m, 1H), 4.32 (m, 1H), 4.12- 3.36 (br m, 4H), 1.89-1.15 (br m, 25H), 0.97 (m, 12H) | ++++ |
| | | octan-3-one | hydrogenation | | |
| 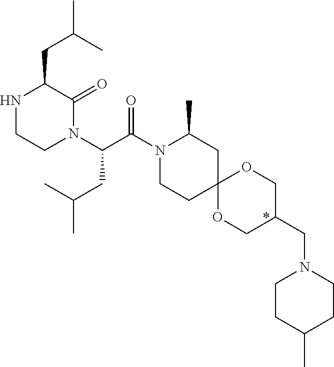 [SMILES: CC(C)C[C@@H]1NCCN([C@@H](CC(C)C)C(=O)N2CCC3(C[C@@H]2C)OCC(CN2CCC(C)CC2)CO3)C1=O] | 253 | (3S)-1-[(2S)-4- methyl-1-oxo-1- [(3s,6s,8S)-8- methyl-3-[(4- methylpiperidin-1- yl)methyl]-1,5- dioxa-9- azaspiro[5.5]undec- an-9-yl]pentan-2- yl]-3-(2- methylpropyl)piper- azin-2-one (^{≠}), isomer Z | (S)-1-[(S)-1- Phenylethyl]-2- methyl-4- piperidinone (AstaTech), ketalization with (2,2-Dimethyl- 1,3-dioxan-5- yl)methanol (Combi-Blocks), tosylation of alcohol, separation of E and Z isomer, substitution of tosylate with 4- Methylpiperidine, catalytic hydrogenation | CD₃OD: δ 5.54 (m, 1H), 3.92 (m, 2H), 3.69 (m, 2H), 2.27 (d, 2H), 0.97 (m, 15H) | ++++ |
| 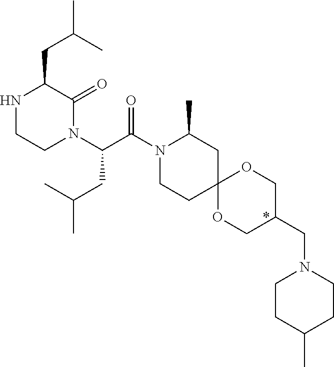 [SMILES: CC(C)C[C@@H]1NCCN([C@@H](CC(C)C)C(=O)N2CCC3(C[C@@H]2C)OCC(CN2CCC(C)CC2)CO3)C1=O] | 254 | (3S)-1-[(2S)-4- methyl-1-oxo-1- [(3r,6r,8S)-8- methyl-3-[(4- methylpiperidin-1- yl)methyl]-1,5- dioxa-9- azaspiro[5.5]undec- an-9-yl]pentan-2- yl]-3-(2- methylpropyl)piper- azin-2-one (^{≠}), isomer E | (S)-1-[(S)-1- Phenylethyl]-2- methyl-4- piperidinone (AstaTech), ketalization with (2,2-Dimethyl- 1,3-dioxan-5- yl)methanol (Combi-Blocks), tosylation of alcohol, separation of E and Z isomer, substitution of tosylate with 4- Methylpiperidine, catalytic hydrogenation | CD₃OD: δ 5.54 (m, 1H), 3.99 (m, 2H), 3.69 (m, 2H), 2.35 (d, 2H), 0.97 (m, 15H) | ++++ |
| 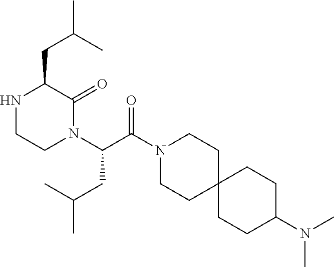 [SMILES: CC(C)C[C@@H]1NCCN([C@@H](CC(C)C)C(=O)N2CCC3(CCC(N(C)C)CC3)CC2)C1=O] | 255 | (S)-1-[(S)-1-({9- (Dimethylamino)- 3-aza-3- spiro[5.5]un- decyl}car- bonyl)-3- methylbutyl]-3- isobutyl-2- piperazinone | tert-Butyl-9-oxo- 3-aza-3- spiro[5.5]undecane- carboxylate (AstaTech), reductive amination with Dimethylamine, BOC removal | CD₃OD: δ 5.55 (m, 1H), 2.30 (s, 6H), 2.22 (m, 1H), 0.96 (m, 12H) | +++ |
| 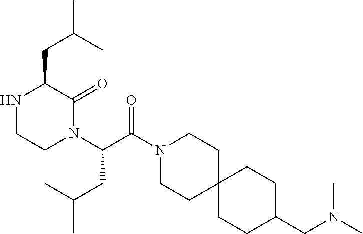 [SMILES: CC(C)C[C@@H]1NCCN([C@@H](CC(C)C)C(=O)N2CCC3(CCC(CN(C)C)CC3)CC2)C1=O] | 256 | (S)-1-[(S)-1-({9- [(Dimethyl- amino)methyl]- 3-aza-3- spiro[5.5]un- decyl}car- bonyl)-3- methylbutyl]-3- isobutyl-2- piperazinone | tert-Butyl 9-oxo- 3-aza-3- spiro[5.5]undecane- carboxylate (AstaTech), Wittig reaction with Methyltriphenyl phosphonium bromide, hydroboration, oxidation to aldehyde, reductive amination with | CD₃OD: δ 5.58 (m, 1H), 3.15 (td, 1H), 2.92 (td, 1H), 2.25 (s, 6H), 0.98 (m, 12H) | ++++ |
| | | | Dimethylamine, | | |
| | | | BOC removal | | |
| 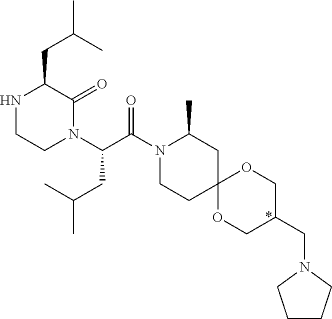 [SMILES: CC(C)C[C@@H]1NCCN([C@@H](CC(C)C)C(=O)N2CCC3(C[C@@H]2C)OCC(CN2CCCC2)CO3)C1=O] | 257 | (3S)-1-[(2S)-4- methyl-1-oxo-1- [(3r,6r,8S)-8- methyl-3- (pyrrolidin-1- ylmethyl)-1,5- dioxa-9- azaspiro[5.5]undec- an-9-yl]pentan-2- yl]-3-(2- methylpropyl)piper- azin-2-one (^{≠}), isomer E | (S)-1-[(S)-1- Phenylethyl]-2- methyl-4- piperidinone (AstaTech), ketalization with (2,2-Dimethyl- 1,3-dioxan-5- yl)methanol (Combi-Blocks), tosylation of alcohol, separation of E and Z isomer, substitution of tosylate with Pyrrolidine, catalytic hydrogenation | CD₃OD: δ 5.53 (m, 1H), 4.45 (m, 1H), 4.10 (m, 2H), 3.75 (m, 2H), 2.33 (m, 1H), 1.92 (m, 4H), 0.96 (m, 12H) | ++++ |
| 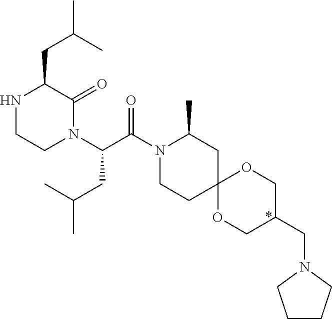 [SMILES: CC(C)C[C@@H]1NCCN([C@@H](CC(C)C)C(=O)N2CCC3(C[C@@H]2C)OCC(CN2CCCC2)CO3)C1=O] | 258 | (3S)-1-[(2S)-4- methyl-1-oxo-1- [(3s,6s,8S)-8- methyl-3- (pyrrolidin-1- ylmethyl)-1,5- dioxa-9- azaspiro[5.5]undec- an-9-yl]pentan-2- yl]-3-(2- methylpropyl)piper- azin-2-one (^{≠}), isomer Z | (S)-1-[(S)-1- Phenylethyl]-2- methyl-4- piperidinone (AstaTech), ketalization with (2,2-Dimethyl- 1,3-dioxan-5- yl)methanol (Combi-Blocks), tosylation of alcohol, separation of E and Z isomer, substitution of tosylate with Pyrrolidine, catalytic hydrogenation | CD₃OD: δ 5.57 (m, 1H), 4.49 (m, 1H), 4.02 (m, 2H), 3.76 (m, 2H), 2.70 (m, 4H), 2.45 (m, 1H), 1.89 (m, 4H), 1.00 (m, 12H) | ++++ |
| 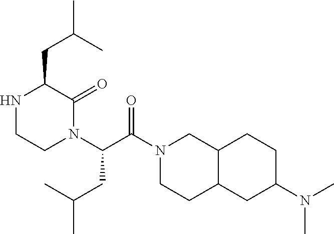 [SMILES: CC(C)C[C@@H]1NCCN([C@@H](CC(C)C)C(=O)N2CCC3CC(N(C)C)CCC3C2)C1=O] | 259 | (S)-1-[(S)-1- {[(4aS,8aR)-6- (Dimethylamino)per- hydroisoquinolin-2- yl]carbonyl}-3- methylbutyl]-3- isobutyl-2- piperazinone | tert-Butyl 6- hydroxy- decahydroiso- quinoline-2- carboxylate (Enamine BB), oxidation to ketone, reductive amination with Dimethylamine, BOC removal | CDCl₃: δ 5.40 (m, 1H), 2.78 (m, 2H), 2.28 and 2.11 (s, 6H), 0.75 (m, 12H) | *+ |
| 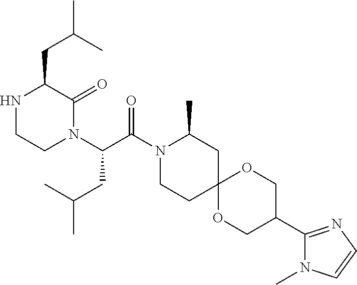 [SMILES: CC(C)C[C@@H]1NCCN([C@@H](CC(C)C)C(=O)N2CCC3(C[C@@H]2C)OCC(c2nccn2C)CO3)C1=O] | 260 | (S)-1-[(S)-1-({(S)-8- Methyl-3-(1- methyl-1H- imidazol-2-yl)-1,5- dioxa-9-aza-9- spiro[5.5]un- decyl}carbonyl)-3- methylbutyl]-3- isobutyl-2- piperazinone | (S)-1-[(S)-1- Phenylethyl]-2- methyl-4- piperidinone (AstaTech), ketalization with (2,2-Dimethyl- 1,3-dioxan-5- yl)methanol (Combi-Blocks), oxidation of alcohol to aldehyde, imidazole formation (with Glyoxal, methylamine and ammonium | CD₃OD: δ 7.02 (d, 1H), 6.91 (d, 1H), 5.55 (m, 1H), 3.71 (s, 3H), 1.08- 0.86 (br m, 12H) | ND |
| | | | acetate), | | |
| | | | catalytic | | |
| | | | hydrogenation | | |
| 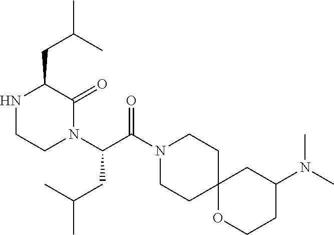 [SMILES: CC(C)C[C@@H]1NCCN([C@@H](CC(C)C)C(=O)N2CCC3(CC2)CC(N(C)C)CCO3)C1=O] | 261 | (S)-1-[(S)-1-({4- (Dimethylamino)- 1-oxa-9-aza-9- spiro[5.5]un- decyl}carbonyl)-3- methylbutyl]-3- isobutyl-2- piperazinone | tert-Butyl 4-oxo- 1-oxa-9-aza-9- spiro[5.5]undecane- carboxylate (Enamine BB), reductive amination with Dimethylamine, BOC removal | CD₃OD: δ 5.60-5.52 (m, 1H), 4.29-4.10 (m, 1H), 2.68 (s, 6H), 0.97 (m, 12H) | +++ |
| 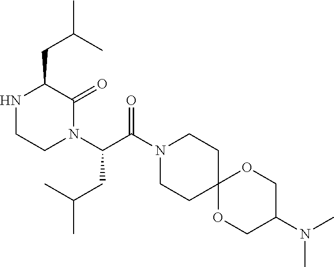 [SMILES: CC(C)C[C@@H]1NCCN([C@@H](CC(C)C)C(=O)N2CCC3(CC2)OCC(N(C)C)CO3)C1=O] | 262 | (S)-1-[(S)-1-({3- (Dimethylamino)- 1,5-dioxa-9-aza-9- spiro[5.5]un- decyl}carbonyl)-3- methylbutyl]-3- isobutyl-2- piperazinone | 2-Amino-1,3- propanediol, Fmoc protection of amine, ketalization with Benzyl 4-oxo-1- piperidinecarbox- ylate, Fmoc removal, reductive amination with formaldehyde, Cbz removal | CD₃OD): δ 5.58 (dd, 1H), 2.48 (m, 1H), 2.34 (s, 6H), 0.96 (m, 12H) | ++++ |
| 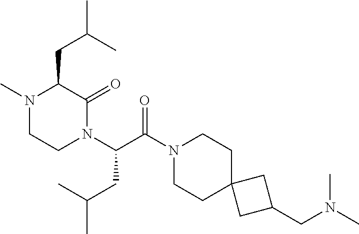 | 263 | (S)-1-[(S)-1-({2- [(Dimethyl- amino)methyl]- 7-aza-7- spiro[3.5]nonyl}car- bonyl)-3- methylbutyl]-3- isobutyl-4-methyl- 2-piperazinone | tert-Butyl 2-oxo- 7-aza-7- spiro[3.5]nonane carboxylate (AstaTech), Wittig reaction with Methyltriphenyl phosphonium bromide, hydroboration, oxidation to aldehyde, reductive amination with | CDCl₃): δ 5.52 (m, 1H), 2.32 (s, 3H), 2.19 (s, 6H), 0.93 (m, 15H) | ++++ |
| | | | Dimethylamine, | | |
| | | | BOC removal | | |
| 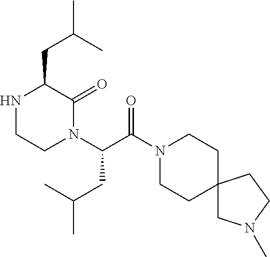 [SMILES: CC(C)C[C@@H]1NCCN([C@@H](CC(C)C)C(=O)N2CCC3(CCN(C)C3)CC2)C1=O] | 264 | (S)-1-[(S)-3-Methyl- 1-{(2-methyl-2,8- diaza-8- spiro[4.5]decyl)car- bonyl}butyl]-3- isobutyl-2- piperazinone | 2-Methyl-2,8- diaza- spiro[4.5]decane (citrate salt, Combi-Blocks) | CD₃OD: δ 5.56 (m, 1H), 3.13 (m, 1H), 2.91 (m, 1H), 2.78 (m, 2H), 2.45 (s, 3H), 1.66 (m, 13H), 0.96 (m, 12H) | +++ |
| 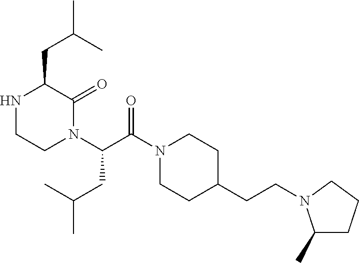 [SMILES: CC(C)C[C@@H]1NCCN([C@@H](CC(C)C)C(=O)N2CCC(CCN3CCC[C@H]3C)CC2)C1=O] | 265 | (S)-1-[(S)-1-[(4-{2- [(R)-2-Methyl-1- pyrrolidinyl]ethyl}- 1- piperidyl)carbonyl]- 3-methylbutyl]-3- isobutyl-2- piperazinone | tert-Butyl 4- (formylmethyl)- 1- piperidinecarbox- ylate, reductive amination with (R)-2- Methylpyrrolidine, BOC removal | CD₃OD: δ 5.56 (m, 1H), 4.08 (m, 1H), 2.66 (m, 1H), 2.47 (m, 1H), 2.22 (m, 2H), 2.02 (m, 1H), 1.67 (m, 14H), 1.13 (m, 5H), 0.96 (m, 12H) | ++++ |
| 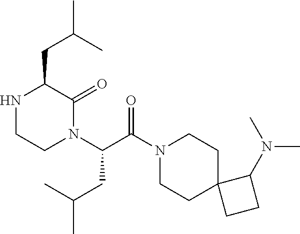 [SMILES: CC(C)C[C@@H]1NCCN([C@@H](CC(C)C)C(=O)N2CCC3(CCC3N(C)C)CC2)C1=O] | 266 | (S)-1-[(S)-1-({1- (Dimethylamino)- 7-aza-7- spiro[3.5]nonyl}car- bonyl)-3- methylbutyl]-3- isobutyl-2- piperazinone | tert-Butyl 1-oxo- 7-aza-7- spiro[3.5]nonane carboxylate (AstaTech), reductive amination with Dimethylamine, BOC removal | CD₃OD: δ 5.56 (m, 1H), 3.13 (m, 1H), 2.91 (m, 1H), 2.78 (m, 2H), 2.45 (s, 3H), 1.66 (m, 13H), 0.96 (m, 12H) | *+ |
| 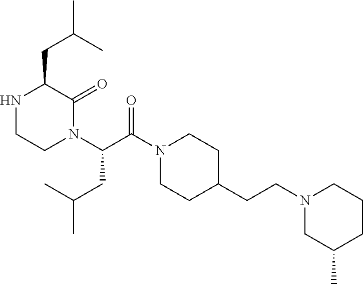 [SMILES: CC(C)C[C@@H]1NCCN([C@@H](CC(C)C)C(=O)N2CCC(CCN3CCC[C@H](C)C3)CC2)C1=O] | 267 | (S)-1-[(S)-1-[(4-{2- [(S)-3-Methyl-1- piperidyl]ethyl}-1- piperidyl)carbonyl]- 3-methylbutyl]-3- isobutyl-2- piperazinone | tert-Butyl 4- (formylmethyl)- 1- piperidinecarbox- ylate, reductive amination with (S)-3- Methylpiperidine (AstaTech), BOC removal | CD₃OD: δ 5.56 (m, 1H), 4.47 (m, 1H), 4.08 (m, 1H), 2.67 (m, 1H), 2.39 (m, 2H), 1.68 (m, 18H), 1.02 (m, 19H) | ++++ |
| 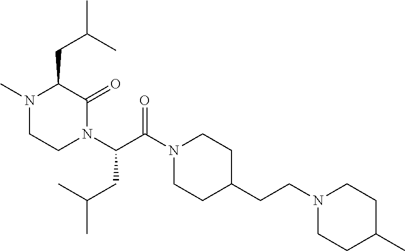 | 268 | (S)-1-[(S)-3-Methyl- 1-({4-[2-(4-methyl- 1-piperidyl)ethyl]- 1- piperidyl}car- bonyl)butyl]-3- isobutyl-4- methyl-2- piperazinone | tert-Butyl 4- (formylmethyl)- 1- piperidinecarbox- ylate, reductive amination with 4- Methylpiperidine, BOC removal | CD₃OD: δ 5.53 (m, 1H), 4.46 (m, 1H), 4.08 (m, 1H), 2.62 (m, 2H), 2.39 (s, 5H), 1.61 (m, 23H), 0.93 (m, 15H) | ++++ |
| 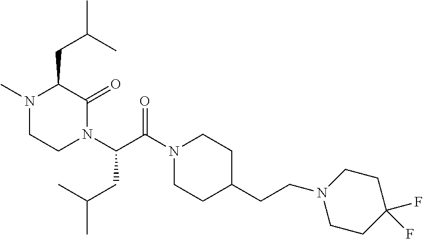 [SMILES: CC(C)C[C@H]1C(=O)N([C@@H](CC(C)C)C(=O)N2CCC(CCN3CCC(F)(F)CC3)CC2)CCN1C] | 269 | (S)-1-[(S)-1-({4-[2- (4,4-Difluoro-1- piperidyl)ethyl]-1- piperidyl}carbonyl)- 3-methylbutyl]-3- isobutyl-4-methyl- 2-piperazinone | tert-Butyl 4- (formylmethyl)- 1- piperidinecarbox- ylate, reductive amination with 4,4- Difluoropiperidine- (AstaTech), BOC removal | CD₃OD: δ 5.53 (m, 1H), 4.47 (m, 1H), 4.09 (m, 1H), 3.05 (m, 2H), 2.37 (s, 3H), 1.75 (m, 15H), 1.00 (m, 15H) | *+ |
| 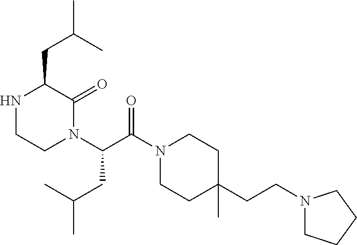 [SMILES: CC(C)C[C@@H]1NCCN([C@@H](CC(C)C)C(=O)N2CCC(C)(CCN3CCCC3)CC2)C1=O] | 270 | (S)-1-[(S)-3-Methyl- 2-({4-methyl-4-[2- (1- pyrrolidinyl)ethyl]- 1- piperidyl}car- bonyl)butyl]-3- isobutyl-2- piperazinone | tert-Butyl 4-(2- hydroxyethyl)-4- methyl-1- piperidinecarbox- ylate (Combi- Blocks), oxidation to aldehyde, reductive amination with Pyrrolidine, BOC removal | CD₃OD: δ 5.55 (m, 1H), 3.19- 3.06 (br m, 1H), 2.00-1.26 (br m, 16H), 1.04 (d, 3H), 0.95 (m, 12H) | +++ |
| 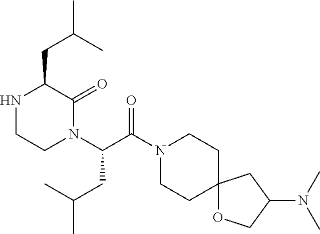 [SMILES: CC(C)C[C@@H]1NCCN([C@@H](CC(C)C)C(=O)N2CCC3(CC2)CC(N(C)C)CO3)C1=O] | 271 | (S)-1-[(S)-1-({3- (Dimethylamino)- 1-oxa-8-aza-8- spiro[4.5]decyl}car- bonyl)-3- methylbutyl]-3- isobutyl-2- piperazinone | tert-Butyl 3-oxo- 1-oxa-8- azaspiro[4.5]dec- ane-8- carboxylate (AstaTech), reductive amination with Dimethylamine, BOC removal | CD₃OD: δ 5.55 (m, 1H), 2.24 (s, 6H), 2.18- 1.98 (br m, 1H), 1.92-1.40 (br m, 11H), 0.95 (m, 12H) | ++++ |
| 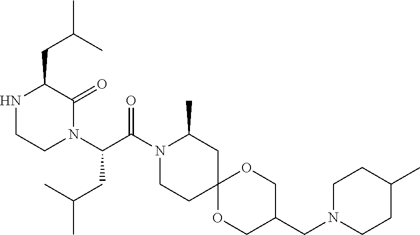 [SMILES: CC(C)C[C@@H]1NCCN([C@@H](CC(C)C)C(=O)N2CCC3(C[C@@H]2C)OCC(CN2CCC(C)CC2)CO3)C1=O] | 272 | (S)-1-[(S)-1-({(S)-8- Methyl-3-[(4- methyl-1- piperidyl)methyl]- 1,5-dioxa-9-aza-9- spiro[5.5]un- decyl}carbonyl)-3- methylbutyl]-3- isobutyl-2- piperazinone | (S)-1-[(S)-1- Phenylethyl]-2- methyl-4- piperidinone (AstaTech), ketalization with (2,2-Dimethyl- 1,3-dioxan-5- yl)methanol (Combi-Blocks), tosylation of alcohol, substitution of tosylate with 4- Methylpiperidine, | CD₃OD: δ 5.54 (m, 1H), 3.68 (m, 2H), 3.11 (m, 1H), 2.55- 1.90 (br m, 7H), 0.95 (m, 15H) | ++++ |
| | | | catalytic | | |
| | | | hydrogenation | | |
| 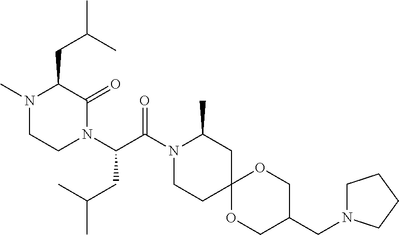 [SMILES: CC(C)C[C@H]1C(=O)N([C@@H](CC(C)C)C(=O)N2CCC3(C[C@@H]2C)OCC(CN2CCCC2)CO3)CCN1C] | 273 | (S)-1-[(S)-1-({(S)-8- Methyl-3-[(1- pyrrolidinyl)methyl]- 1,5-dioxa-9-aza- 9- spiro[5.5]un- decyl}carbonyl)-3- methylbutyl]-3- isobutyl-4-methyl- 2-piperazinone | (S)-1-[(S)-1- Phenylethyl]-2- methyl-4- piperidinone (AstaTech), ketalization with (2,2-Dimethyl- 1,3-dioxan-5- yl)methanol (Combi-Blocks), tosylation of alcohol, substitution of tosylate with Pyrrolidine, | CDCl₃: δ 5.50 (t, 1H), 3.00- 2.35 (br m, 10H), 2.31 (s, 3H), 1.00-0.80 (br m, 12H) | ++++ |
| | | | catalytic | | |
| | | | hydrogenation | | |
| 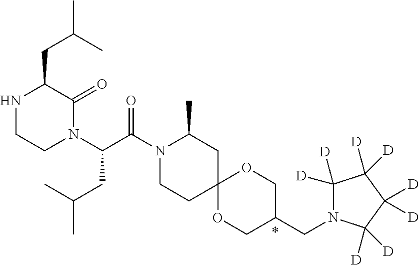 [SMILES: [2H]C1([2H])N(CC2COC3(CCN(C(=O)[C@H](CC(C)C)N4CCN[C@@H](CC(C)C)C4=O)[C@@H](C)C3)OC2)C([2H])([2H])C([2H])([2H])C1([2H])[2H]] | 274 | (3S)-1-[(2S)-4- methyl-1-oxo-1- [(3s,6s,8S)-8- methyl-3- [(²H₈)pyrrolidin-1- ylmethyl]-1,5- dioxa-9- azaspiro[5.5]undec- an-9-yl]pentan-2- y]-3-(2- methylpropyl)piper- azin-2-one (^{≠}), isomer Z | (S)-1-[(S)-1- Phenylethyl]-2- methyl-4- piperidinone (AstaTech), ketalization with (2,2-Dimethyl- 1,3-dioxan-5- yl)methanol (Combi-Blocks), tosylation of alcohol, separation of E and Z isomer, substitution of tosylate with Pyrrolidine-d₈, | CD₃OD: δ 5.54 (m, 1H), 2.46 (m, 3H), 2.07 (m, 2H), 0.96 (m, 12H) | ++++ |
| | | | catalytic | | |
| | | | hydrogenation | | |
| 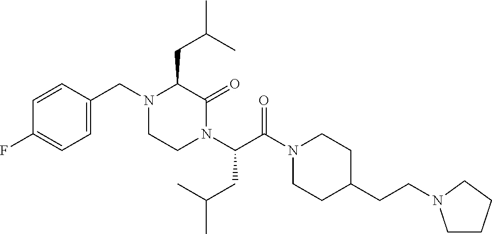 [SMILES: CC(C)C[C@H]1C(=O)N([C@@H](CC(C)C)C(=O)N2CCC(CCN3CCCC3)CC2)CCN1Cc1ccc(F)cc1] | 275 | (S)-1-[(S)-3-Methyl- 1-({4-[2-(1- pyrrolidinyl)ethyl]- 1- piperidyl}car- bonyl)butyl]-4-[(p- fluorophenyl)meth- yl]-3-isobutyl-2- piperazinone | tert-Butyl 4- (formylmethyl)- 1- piperidinecarbox- ylate, reductive amination with Pyrrolidine, BOC removal | CD₃OD: δ 7.24 (m, 2H), 6.97 (m, 2H), 5.56 (m, 1H), 4.14 (t, 1H), 0.89 (m, 12H) | ++++ |
| 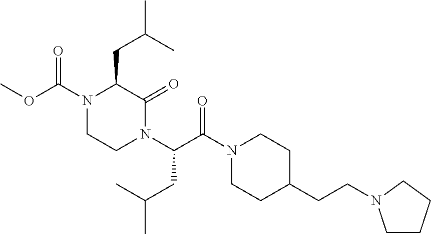 [SMILES: COC(=O)N1CCN([C@@H](CC(C)C)C(=O)N2CCC(CCN3CCCC3)CC2)C(=O)[C@@H]1CC(C)C] | 276 | Methyl (S)-4-[(S)-3- methyl-1-({4-[2-(1- pyrrolidinyl)ethyl]- 1- piperidyl}car- bonyl)butyl]-2- isobutyl-3-oxo-1- piperazinecar- boxylate | tert-Butyl 4- (formylmethyl)- 1- piperidinecarbox- ylate, reductive amination with Pyrrolidine, BOC removal | CD₃OD: δ 5.55 (m, 1H), 3.74 (d, 3H), 3.05 (m, 1H), 1.46 (m, 4H), 0.97 (m, 12H) | ++++ |
| 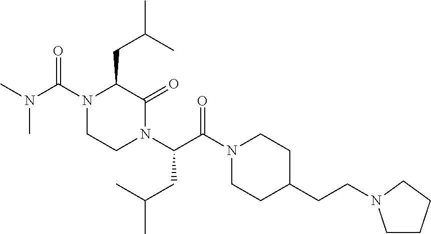 [SMILES: CC(C)C[C@@H](C(=O)N1CCC(CCN2CCCC2)CC1)N1CCN(C(=O)N(C)C)[C@@H](CC(C)C)C1=O] | 277 | (S)-1-[(S)-3-Methyl- 1-({4-[2-(1- pyrrolidinyl)ethyl]- 1- piperidyl}car- bonyl)butyl]-4- (dimethylamino)car- bonyl-3-isobutyl- 2-piperazinone | tert-Butyl 4- (formylmethyl)- 1- piperidinecarbox- ylate, reductive amination with Pyrrolidine, BOC removal | CD₃OD: δ 5.55 (m, 1H), 4.48 (d, 2H), 4.00 (m, 1H), 2.81 (d, 6H), 0.97 (m, 12H) | *+ |
| 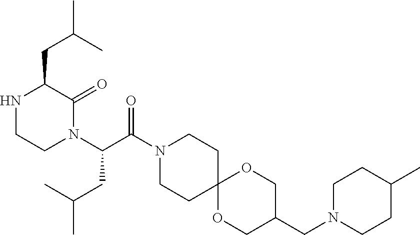 [SMILES: CC(C)C[C@@H]1NCCN([C@@H](CC(C)C)C(=O)N2CCC3(CC2)OCC(CN2CCC(C)CC2)CO3)C1=O] | 278 | (S)-1-[(S)-3-Methyl- 1-({3-[(4-methyl-1- piperidyl)methyl]- 1,5-dioxa-9-aza-9- spiro[5.5]un- decyl}car- bonyl)butyl]-3- isobutyl-2- piperazinone | Benzyl 4-oxo-1- piperidinecarbox- ylate, ketalization with (2,2-Dimethyl- 1,3-dioxan-5- yl)methanol (Combi-Blocks), tosylation of alcohol, substitution of tosylate using 4- Methylpiperidine, Cbz removal | CD₃OD: δ 5.59 (t, 1H), 3.91 (br m, 1H), 2.23 (t, 2H), 1.69-1.41 (m, 8H), 1.39-1.25 (m, 1H) 0.97 (m, 15H) | ++++ |
| 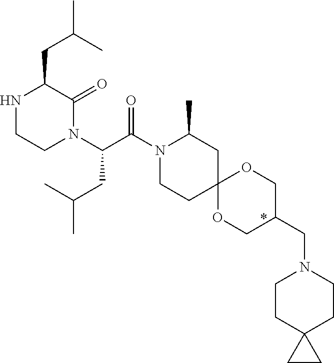 [SMILES: CC(C)C[C@@H]1NCCN([C@@H](CC(C)C)C(=O)N2CCC3(C[C@@H]2C)OCC(CN2CCC4(CC2)CC4)CO3)C1=O] | 279 | (3S)-1-[(2S)-4- methyl-1-oxo-1- [(3s,6s,8S)-3-{6- azaspiro[2.5]octan- 6-ylmethyl}-8- methyl-1,5-dioxa- 9- azaspiro[5.5]undec- an-9-yl]pentan-2- yl]-3-(2- methylpropyl)piper- azin-2-one (^{≠}), isomer Z | (S)-1-[(S)-1- Phenylethyl]-2- methyl-4- piperidinone (AstaTech), ketalization with (2,2-Dimethyl- 1,3-dioxan-5- yl)methanol (Combi-Blocks), tosylation of alcohol, separation of E and Z isomer, substitution of tosylate with 6- Azaspiro[2.5]oc- tane (AstaTech), cattalytic hydrogenation | CD₃OD: δ 5.45 (t, 1H), 4.63 (m, 1H), 1.89 (m, 1H), 0.83 (m, 12H), 0.16 (s, 4H) | ++++ |
| 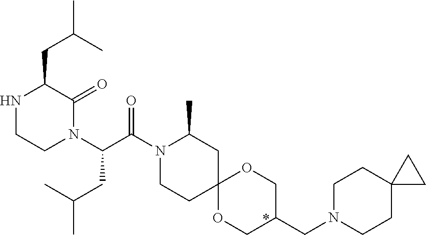 [SMILES: CC(C)C[C@@H]1NCCN([C@@H](CC(C)C)C(=O)N2CCC3(C[C@@H]2C)OCC(CN2CCC4(CC2)CC4)CO3)C1=O] | 280 | (3S)-1-[(2S)-4- methyl-1-oxo-1- [(3r,6r,8S)-3-{6- azaspiro[2.5]octan- 6-ylmethyl}-8- methyl-1,5-dioxa-9- azaspiro[5.5]undec- an-9-yl]pentan-2- yl]-3-(2- methylpropyl)piper- azin-2-one (^{≠}), isomer E | (S)-1-[(S)-1- Phenylethyl]-2- methyl-4- piperidinone (AstaTech), ketalization with (2,2-Dimethyl- 1,3-dioxan-5- yl)methanol (Combi-Blocks), tosylation of alcohol, separation of E and Z isomer, substitution of | CD₃OD: δ 5.54 (m, 1H), 4.01 (m, 2H), 3.71 (m, 2H), 0.97 (m, 12H), 0.30 (s, 4H) | ++++ |
| | | | tosylate with 6- | | |
| | | | Azaspiro[2.5]oc- | | |
| | | | tane (AstaTech), | | |
| | | | catalytic | | |
| | | | hydrogenation | | |
| 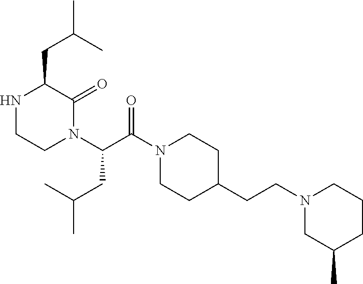 [SMILES: CC(C)C[C@@H]1NCCN([C@@H](CC(C)C)C(=O)N2CCC(CCN3CCC[C@@H](C)C3)CC2)C1=O] | 281 | (S)-1-[(S)-1-[(4-{2- [(R)-3-Methyl-1- piperidyl]ethyl}-1- piperidyl)carbonyl]- 3-methylbutyl]-3- isobutyl-2- piperazinone | tert-Butyl 4- (formylmethyl)- 1- piperidinecarbox- ylate, reductive amination with (R)-3- Methylpiperidine (AstaTech), BOC removal | CD₃OD: δ 5.59 (m, 1H), 4.51 (t, 1H), 2.04 (m, 1H), 0.99 (m, 12H), 0.94 (d, 3H) | ++++ |
| 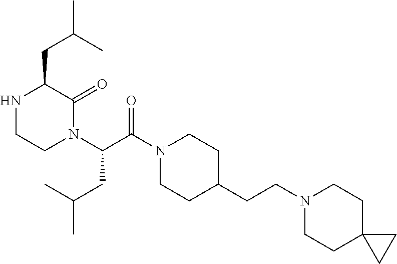 [SMILES: CC(C)C[C@@H]1NCCN([C@@H](CC(C)C)C(=O)N2CCC(CCN3CCC4(CC3)CC4)CC2)C1=O] | 282 | (S)-1-[(S)-1-[(4-{2- (6-Aza-6- spiro[2.5]octyl)eth- yl}-1- piperidyl)carbonyl]- 3-methylbutyl]-3- isobutyl-2- piperazinone | tert-Butyl 4- (formylmethyl)- 1- piperidinecarbox- ylate, reductive amination with 6- Azaspiro[2.5]oc- tane (AstaTech), BOC removal | CD₃OD: δ 5.58 (m, 1H), 4.48 (t, 1H), 3.35 (m, 2H), 3.11 (m, 2H), 0.97 (m, 12H), 0.36 (s, 4H) | ++++ |
| 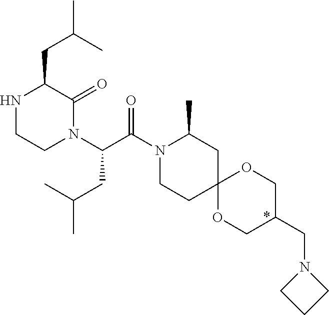 [SMILES: CC(C)C[C@@H]1NCCN([C@@H](CC(C)C)C(=O)N2CCC3(C[C@@H]2C)OCC(CN2CCC2)CO3)C1=O] | 283 | (3S)-1-[(2S)-4- methyl-1-oxo-1- [(3r,6r,8S)-3- (azetidin-1- ylmethyl)-8- methyl-1,5-dioxa- 9- azaspiro[5.5]undec- an-9-yl]pentan-2- yl]-3-(2- methylpropyl)piper- azin-2-one (^{≠}), isomer E | (S)-1-[(S)-1- Phenylethyl]-2- methyl-4- piperidinone (AstaTech), ketalization with (2,2-Dimethyl- 1,3-dioxan-5- yl)methanol (Combi-Blocks), tosylation of alcohol, separation of E and Z isomer, substitution of tosylate with Azetidine, catalytic hydrogenation | CD₃OD: δ 5.54 (m, 1H), 3.96 (m, 2H), 3.67 (m, 2H), 2.51 (d, 2H), 1.89- 1.42 (m, 5H), 0.97 (m, 12H) | ++++ |
| 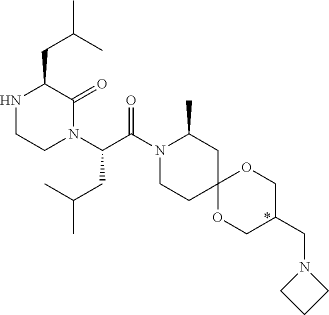 [SMILES: CC(C)C[C@@H]1NCCN([C@@H](CC(C)C)C(=O)N2CCC3(C[C@@H]2C)OCC(CN2CCC2)CO3)C1=O] | 284 | (3S)-1-[(2S)-4- methyl-1-oxo-1- [(3s,6s,8S)-3- (azetidin-1- ylmethyl)-8- methyl-1,5-dioxa- 9- azaspiro[5.5]undec- an-9-yl]pentan-2- yl]-3-(2- methylpropyl)piper- azin-2-one (^{≠}), isomer Z | (S)-1-[(S)-1- Phenylethyl]-2- methyl-4- piperidinone (AstaTech), ketalization with (2,2-Dimethyl- 1,3-dioxan-5- yl)methanol (Combi-Blocks), tosylation of alcohol, separation of E and Z isomer, substitution of tosylate with Azetidine, catalytic hydrogenation | CD₃OD: δ 5.54 (m, 1H), 3.91 (m, 2H), 3.69 (m, 2H), 2.52 (d, 2H), 1.7- 1.44 (m, 5H), 0.97 (m, 12H) | ++++ |
| 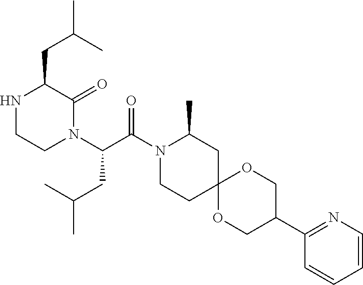 [SMILES: CC(C)C[C@@H]1NCCN([C@@H](CC(C)C)C(=O)N2CCC3(C[C@@H]2C)OCC(c2ccccn2)CO3)C1=O] | 285 | (S)-1-[(S)-1-({(S)-8- Methyl-3-(2- pyridyl)-1,5-dioxa- 9-aza-9- spiro[5.5]un- decyl}carbonyl)-3- methylbutyl]-3- isobutyl-2- piperazinone | 2-(2-Pyridyl)-1,3- propanediol (Combi-Blocks), ketalization with (S)-1-[(S)-1- Phenylethyl]-2- methyl-4- piperidinone (AstaTech), catalytic hydrogenation | CD₃OD): δ 8.51 (s, 1H), 7.80 (t, 1H), 7.51 (dd, 1H), 7.31 (t, 1H), 5.56 (m, 1H), 0.97 (m, 12H) | ND |
| 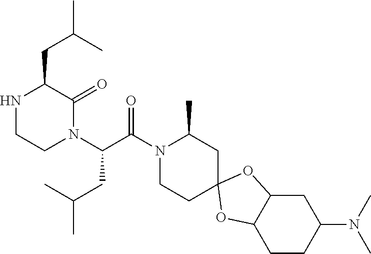 [SMILES: CC(C)C[C@@H]1NCCN([C@@H](CC(C)C)C(=O)N2CCC3(C[C@@H]2C)OC2CCC(N(C)C)CC2O3)C1=O] | 286 | (S)-1-[(S)-1-({(2′S)- 5-(Dimethylamino)- 2′-methyl- 3a,4,5,6,7,7a- hexahydrospiro[in- dene-2,4′- piperidin]-1′- yl}carbonyl)-3- methylbutyl]-3- isobutyl-2- piperazinone | 4-Amino-1,2- cyclohexanediol (Enamine BB), reductive amination with formaldehyde ketalization with (S)-1-[(S)-1- Phenylethyl]-2- methyl-4- piperidinone (AstaTech), catalytic hydrogenation | CD₃OD: δ 5.55 (m, 1H), 3.12 (m, 1H), 2.91 (m, 1H), 2.28 (br, 3, 6H), 1.94-1.44 (m, 13H), 0.96 (m, 12H) | ++++ |
| 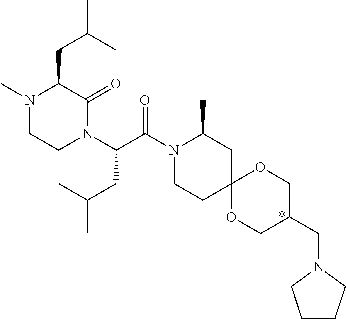 [SMILES: CC(C)C[C@H]1C(=O)N([C@@H](CC(C)C)C(=O)N2CCC3(C[C@@H]2C)OCC(CN2CCCC2)CO3)CCN1C] | 287 | (3S)-4-methyl-1- [(2S)-4-methyl-1- oxo-1-[(3s,6s,8S)-8- methyl-3- (pyrrolidin-1- ylmethyl)-1,5- dioxa-9- azaspiro[5.5]undec- an-9-yl]pentan-2- yl]-3-(2- methylpropyl)piper- azin-2-one (^{≠}), isomer Z | (S)-1-[(S)-1- Phenylethyl]-2- methyl-4- piperidinone (AstaTech), ketalization with (2,2-Dimethyl- 1,3-dioxan-5- yl)methanol (Combi-Blocks), tosylation of alcohol, separation of E and Z isomer, substitution of tosylate with Pyrrolidine, catalytic hydrogenation | CDCl₃: δ 5.43 (t, 1H), 4.66 (m, 1H), 3.57 (m, 2H), 2.23 (s, 3H), 2.03 (m, 1H), 0.83 (m, 12H) | ++++ |
| 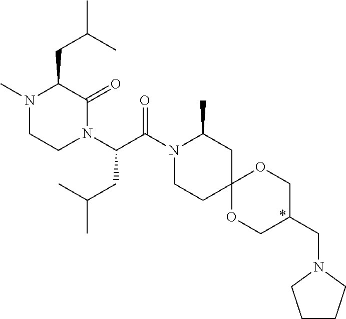 [SMILES: CC(C)C[C@H]1C(=O)N([C@@H](CC(C)C)C(=O)N2CCC3(C[C@@H]2C)OCC(CN2CCCC2)CO3)CCN1C] | 288 | (3S)-4-methyl-1- [(2S)-4-methyl-1- oxo-1-[(3r,6r,8S)-8- methyl-3- (pyrrolidin-1- ylmethyl)-1,5- dioxa-9- azaspiro[5.5]undec- an-9-yl]pentan-2- yl]-3-(2- methylpropyl)piper- azin-2-one (^{≠}), isomer E | (S)-1-[(S)-1- Phenylethyl]-2- methyl-4- piperidinone (AstaTech), ketalization with (2,2-Dimethyl- 1,3-dioxan-5- yl)methanol (Combi-Blocks), tosylation of alcohol, separation of E and Z isomer, substitution of tosylate with Pyrrolidine, catalytic hydrogenation | CD₃OD: δ 5.45 (t, 1H), 4.67 (m, 1H), 3.92 (m, 2H), 3.63 (m, 2H), 2.26 (s, 3H), 1.92 (m, 1H), 0.86 (m, 12H) | ++++ |
| 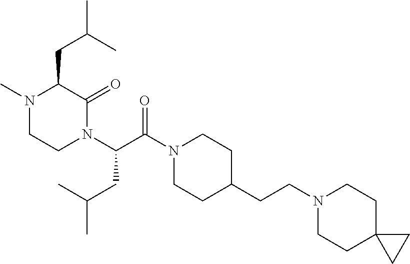 [SMILES: CC(C)C[C@H]1C(=O)N([C@@H](CC(C)C)C(=O)N2CCC(CCN3CCC4(CC3)CC4)CC2)CCN1C] | 289 | (S)-1-[(S)-1-[(4-{2- (6-Aza-6- spiro[2.5]octyl)eth- yl}-1- piperidyl)carbonyl]- 3-methylbutyl]-3- isobutyl-4-methyl- 2-piperazinone | tert-Butyl 4- (formylmethyl)- 1- piperidinecarbox- ylate, reductive amination with 6- Azaspiro[2.5]oc- tane (AstaTech), BOC removal | CD₃OD: δ 5.55 (m, 1H), 4.48 (t, 1H), 2.38 (s, 3H), 0.33 (s, 4H) | ++++ |
| 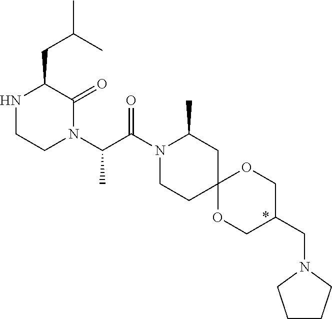 [SMILES: CC(C)C[C@@H]1NCCN([C@@H](C)C(=O)N2CCC3(C[C@@H]2C)OCC(CN2CCCC2)CO3)C1=O] | 290 | (3S)-3-(2- methylpropyl)-1- [(2S)-1-oxo-1- [(3s,6s,8S)-8- methyl-3- (pyrrolidin-1- ylmethyl)-1,5- dioxa-9- azaspiro[5.5]undec- an-9-yl]propan-2- yl)piperazin-2-one (^{≠}), isomer Z | (S)-1-[(S)-1- Phenylethyl]-2- methyl-4- piperidinone (AstaTech), ketalization with (2,2-Dimethyl- 1,3-dioxan-5- yl)methanol (Combi-Blocks), tosylation of alcohol, separation of E and Z isomer, substitution of tosylate with Pyrrolidine, catalytic hydrogenation | CD₃OD: δ 5.45 (t, 1H), 4.63 (m, 1H), 1.89 (m, 1H), 0.83 (m, 12H), 0.16 (s, 4H) | ++++ |
| 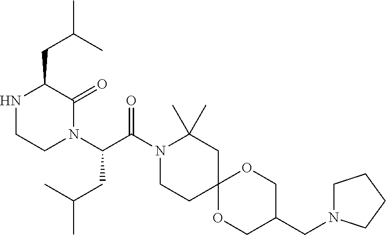 [SMILES: CC(C)C[C@@H]1NCCN([C@@H](CC(C)C)C(=O)N2CCC3(CC2(C)C)OCC(CN2CCCC2)CO3)C1=O] | 291 | (S)-1-[(S)-1-({8,8- Dimethyl-3-[(1- pyrrolidinyl)methyl]- 1,5-dioxa-9-aza- 9- spiro[5.5]un- decyl}carbonyl)-3- methylbutyl]-3- isobutyl-2- piperazinone | 1-Benzyl-2,2- dimethyl-4- piperidinone (AstaTech), ketalization with (2,2-Dimethyl- 1,3-dioxan-5- yl)methanol (Combi-Blocks), tosylation of alcohol, substitution of tosylate with Pyrrolidine, catalytic | CDCl₃: δ 5.41 (m, 1H), 3.85 (m, 2H), 3.55 (m, 2H), 1.38 (s, 6H 0.84 (m, 12H) | *+ |
| | | | hydrogenation | | |
| 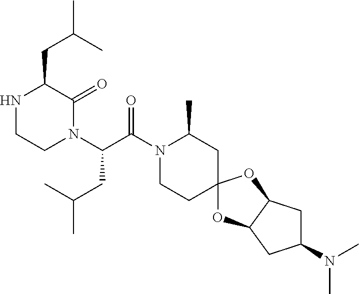 | 292 | (S)-1-[(S)-1- ({(1R,5S,2′S)-7- (Dimethylamino)- 2′-methylspiro[2.4-di- oxabicyclo[3.3.0]oc- tane-3,4′- piperidin]-1′- yl}carbonyl)-3- methylbutyl]-3- isobutyl-2- piperazinone | (1R,2S,4R)-4- Amino-1,2- cyclopentanediol (HCl salt, Enamine BB), reductive amination with Formaldehyde, ketalization with (S)-1-[(S)-1- Phenylethyl]-2- methyl-4- piperidinone (AstaTech), catalytic hydrogenation | CD₃OD: δ 5.53 (m, 1H), 2.63 (m, 1H), 2.40- 2.17 (br m, 8H), 0.96 (m, 12H) | ND |
| 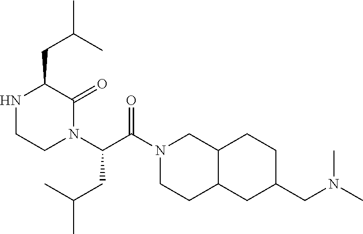 [SMILES: CC(C)C[C@@H]1NCCN([C@@H](CC(C)C)C(=O)N2CCC3CC(CN(C)C)CCC3C2)C1=O] | 293 | (S)-1-[(S)-1-({6- [(Dimethyl- amino)methyl]per- hydroiso- quinol-2- yl}carbonyl)-3- methylbutyl]-3- isobutyl-2- piperazinone | tert-Butyl 6- hydroxy- decahydroiso- quinoline-2- carboxylate (Enamine BB), oxidation to ketone, Wittig reaction with Methyltriphenyl phosphonium bromide, hydroboration, oxidation to aldehyde, | CDCl₃: δ 5.48 (t, 1H), 3.05 (m, 2H), 2.86 (m, 2H), 2.22 (s, 6H), 0.84 (m, 12H) | *+ |
| | | | reductive | | |
| | | | amination with | | |
| | | | Dimethylamine, | | |
| | | | BOC removal | | |
| 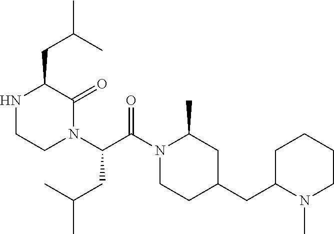 [SMILES: CC(C)C[C@@H]1NCCN([C@@H](CC(C)C)C(=O)N2CCC(CC3CCCCN3C)C[C@@H]2C)C1=O] | 294 | (S)-1-[(S)-1-({(2S)- 2-Methyl-4-[(1- methyl-2- piperidyl)methyl]- 1- piperidyl}carbonyl)- 3-methylbutyl]-3- isobutyl-2- piperazinone | tert-Butyl 2- (hydroxymethyl)- 1- piperidinecarbox- ylate (Combi- Blocks), Mitsunobu reaction with 1,3- Benzothiazole-2- thiol (Combi- Blocks), oxidation to sulfone, Julia- Kocienski | CD₃OD: δ 5.37 (t, 1H), 4.21 (m, 1H), 3.84 (m, 1H), 2.41 (s, 3H), 0.84 (m, 12H) | *+ |
| | | | olefination with | | |
| | | | with (S)-1-[(S)-1- | | |
| | | | Phenylethyl]-2- | | |
| | | | methyl-4- | | |
| | | | piperidinone | | |
| | | | (AstaTech), BOC | | |
| | | | removal, | | |
| | | | reductive | | |
| | | | amination with | | |
| | | | Formaldehyde, | | |
| | | | catalytic | | |
| | | | hydrogenation | | |
| 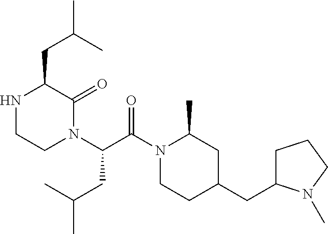 [SMILES: CC(C)C[C@@H]1NCCN([C@@H](CC(C)C)C(=O)N2CCC(CC3CCCN3C)C[C@@H]2C)C1=O] | 295 | (S)-1-[(S)-1-({(2S)- 2-Methyl-4-[(1- methyl-2- pyrrolidinyl)methyl]- 1- piperidyl}carbonyl)- 3-methylbutyl]-3- isobutyl-2- piperazinone | tert-Butyl 2- (hydroxymethyl)- 1- pyrrolidinecarbox- ylate (TCI America), Mitsunobu reaction with 1,3- Benzothiazole-2- thiol (Combi- Blocks), oxidation to sulfone, Julia- Kocienski | CD₃OD: δ 5.37 (t, 1H), 4.16 (m, 1H), 2.78 (m, 2H), 2.24 (s, 3H), 0.80 (m, 12H) | *+ |
| | | | olefination with | | |
| | | | with (S)-1-[(S)-1- | | |
| | | | Phenylethyl]-2- | | |
| | | | methyl-4- | | |
| | | | piperidione | | |
| | | | (AstaTech), BOC | | |
| | | | removal, | | |
| | | | reductive | | |
| | | | amination with | | |
| | | | Formaldehyde, | | |
| | | | catalytic | | |
| | | | hydrogenation | | |
| 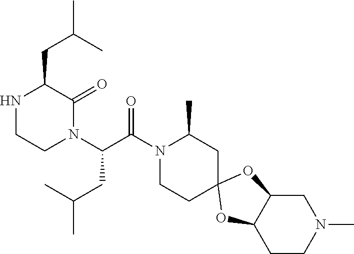 | 296 | (S)-1-[(S)-1- ({(3aS,7aR,2′S)-5- Methyl-2′-methyl- 3a,4,5,6,7,7a- hexahydrospiro[in- dene-2,4′- piperidin]-1′- yl}carbonyl)-3- methylbutyl]-3- isobutyl-2- piperazinone | (3S,4R)-3,4- Piperidinediol (HCl salt, AstaTech), reductive amination with Formaldehyde, ketalization with (S)-1-[(S)-1- Phenylethyl]-2- methyl-4- piperidinone (AstaTech), catalytic hydrogenation | CDCl₃: δ 5.54 (m, 1H), 4.14 (m, 5H), 2.25 (s, 3H), 0.92 (m, 15H) | ND |
| 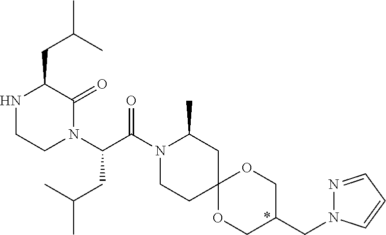 [SMILES: CC(C)C[C@@H]1NCCN([C@@H](CC(C)C)C(=O)N2CCC3(C[C@@H]2C)OCC(Cn2cccn2)CO3)C1=O] | 297 | (3S)-1-[(2S)-4- Methyl-1-oxo-1- [(3s,6s,8S)-8- methyl-3-(1H- pyrazol-1- ylmethyl)-1,5- dioxa-9- azaspiro[5.5]undec- an-9-yl]pentan-2- yl]-3-(2- methylpropyl)piper- azin-2-one (^{≠}), isomer Z | (S)-1-[(S)-1- Phenylethyl]-2- methyl-4- piperidinone (AstaTech), ketalization with (2,2-Dimethyl- 1,3-dioxan-5- yl)methanol (Combi-Blocks), tosylation of alcohol, separation of E and Z isomer, subsitution of | CDCl₃: δ 7.46 (s, 1H), 7.36 (s, 1H), 6.20 (s, 1H), 5.50 (t, 1H), 0.92 (m, 15H) | *+ |
| | | | tosylate with 1H- | | |
| | | | Pyrazole, | | |
| | | | catalytic | | |
| | | | hydrogenation | | |
| 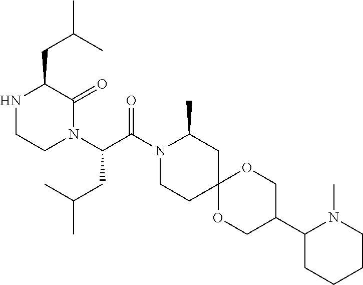 [SMILES: CC(C)C[C@@H]1NCCN([C@@H](CC(C)C)C(=O)N2CCC3(C[C@@H]2C)OCC(C2CCCCN2C)CO3)C1=O] | 298 | (S)-1-[(S)-1-({(S)-8- Methyl-3-(1- methyl-2- piperidyl)-1,5- dioxa-9-aza-9- spiro[5.5]un- decyl}carbonyl)-3- methylbutyl]-3- isobutyl-2- piperazinone | 2-(1- Methylpiperidin- 2-yl)propane- 1,3-diol (Oakwood Chemical), ketalization with (S)-1-[(S)-1- Phenylethyl]-2- methyl-4- piperidinone (AstaTech), catalytic hydrogenation | CD₃OD: δ 5.58- 5.49 (m, 1H), 3.17-3.07 (m, 1H), 2.38 (s, 3H), 1.00-0.92 (m, 12H) | ++++ |
| 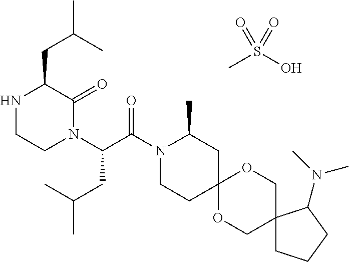 | 299 | (S)-1-[(S)-1-({(10S)- 1- (Dimethylamino)- 10-methyl-7,14- dioxa-11-aza-11- dispiro[4.2.5.2]pen- dtaecyl}carbonyl)- 3-methylbutyl]-3- isobutyl-2- piperazinone, methanesulfonic acid salt | Methyl 2- oxocyclopentane carboxylate, alkylation with Benzyloxymethyl chloride, reduction with LAH, catalytic hydrogenation, ketalization with (S)-1-[(S)-1- Phenylethyl]-2- methyl-4- piperidinone (AstaTech), mesylation of | CD₃OD: δ 5.60- 5.48 (m, 1H), 4.16-4.05 (m, 2H), 3.01-2.88 (m, 6H), 1.06- 0.94 (m, 12H) | *+ |
| | | | alcohol, | | |
| | | | substitution of | | |
| | | | mesylate with | | |
| | | | Dimethylamine, | | |
| | | | catalytic | | |
| | | | hydrogenation | | |
| 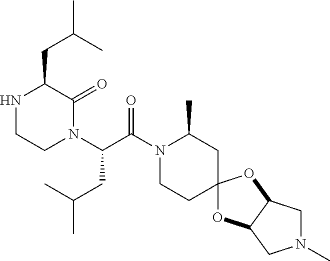 | 300 | (S)-1-[(S)-1-({(2′S)- 7-Methyl-2′- methylspiro[2.4- dioxa-7- azabicyclo[3.3.0]oc- tane-3,4′- piperidin]-1′- yl}carbonyl)-3- methylbutyl]-3- isobutyl-2- piperazinone | cis-Pyrrolidine- 3,4-diol (HCl salt, Combi-Blocks), reductive amination with Formaldehyde, ketalization with (S)-1-[(S)-1- Phenylethyl]-2- methyl-4- piperidinone (AstaTech), catalytic hydrogenation | CD₃OD: δ 5.56 (m, 1H), 4.76 (m, 1H), 4.68 (m, 1H), 2.37 (s, 3H), 0.96 (m, 12H) | ND |
| 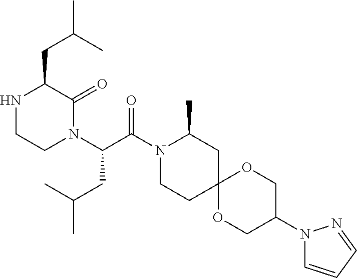 [SMILES: CC(C)C[C@@H]1NCCN([C@@H](CC(C)C)C(=O)N2CCC3(C[C@@H]2C)OCC(n2cccn2)CO3)C1=O] | 301 | (S)-1-[(S)-1-({(S)-8- Methyl-3-(1H- pyrazol-1-yl)-1,5- dioxa-9-aza-9- spiro[5.5]un- decyl}carbonyl)-3- methylbutyl]-3- isobutyl-2- piperazinone | Glycerol, protection of primary alcohols with tert- Butyldimethylsilyl chloride, mesylation of secondary alcohol, substitution of mesylate with 1H-Pyrazole, TBDMS ether removal, ketalization with (S)-1-[(S)-1- Phenylethyl]-2- | CDCl₃: δ 7.52 (b s, 1H), 745 (b s, 1H), 6.26 (b s, 1H), 5.55 (m, 1H), 0.92 (m, 15H) | *+ |
| | | | methyl-4- | | |
| | | | piperidinone | | |
| | | | (AstaTech), | | |
| | | | catalytic | | |
| | | | hydrogenation | | |
| 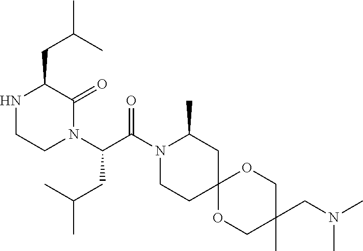 [SMILES: CC(C)C[C@@H]1NCCN([C@@H](CC(C)C)C(=O)N2CCC3(C[C@@H]2C)OCC(C)(CN(C)C)CO3)C1=O] | 302 | (S)-1-[(S)-1-({(S)-3- [(Dimethyl- amino)methyl]-3,8- dimethyl-1,5- dioxa-9-aza-9- spiro[5.5]un- decyl}carbonyl)-3- methylbutyl]-3- isobutyl-2- piperazinone | 2- (Hydroxymethyl)- 2-methyl-1,3- propanediol (Combi-Blocks), ketalization with (S)-1-[(S)-1- Phenylethyl]-2- methyl-4- piperidinone (AstaTech), oxidation of alcohol to aldehyde, reductive amination with | CD₃OD: δ 5.54 (m, 1H), 4.58 (m, 2H), 2.34 (m, 6H), 1.52 (m, 10H), 0.95 (m, 15H) | ++++ |
| | | | Dimethylamine, | | |
| | | | catalytic | | |
| | | | hydrogenation | | |

TABLE 3
Compounds made through Method B1 and B2

| | | | | Character- | |
|---|---|---|---|---|---|
| | | | | istic | |
| | Ex. | | | ¹H NMR | Ac- |
| | Cmpd | | Precursor III | signals | tiv- |
| Structure | # | name | synthesis | (300 MHz) | ity |
| 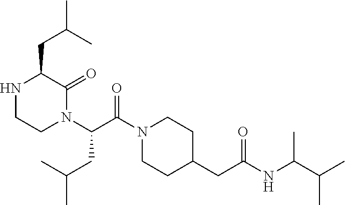 [SMILES: CC(C)C[C@@H]1NCCN([C@@H](CC(C)C)C(=O)N2CCC(CC(=O)NC(C)C(C)C)CC2)C1=O] | 136 | (S)-1-[(S)-1-({4-[2-(1,2- Dimethyl- propylamino)- 2-oxoethyl]-1- piperidyl}carbonyl)-3- methylbutyl]-3- isobutyl-2-piperazinone | (B1) IIIb-Z = CHCH₂C(O)OCH₃ (Combi-Blocks), (B2) hydrolysis, amide coupling | CD₃OD: δ 5.57 (m, 1H), 4.48 (t, 1H), 4.09 (m, 1H), 0.96 (m, 18H) | ++ |
| 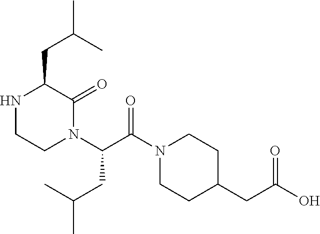 [SMILES: CC(C)C[C@@H]1NCCN([C@@H](CC(C)C)C(=O)N2CCC(CC(=O)O)CC2)C1=O] | 137 | (1-{(S)-2- [(S)-3-Isobutyl- 2-oxo-1- piperazinyl]-4- methylvaleryl}-4- piperidyl)acetic acid | (B1) IIIb-Z = CHCH₂C(O)OCH₂CH₃ (Combi-Blocks), (B2) hydrolysis | CD₃OD: δ 5.48 (m, 1H), 4.39 (t, 1H), 2.61 (m, 2H), 1.62 (m, 11H), 0.88 (m, 12H) | +* |
| 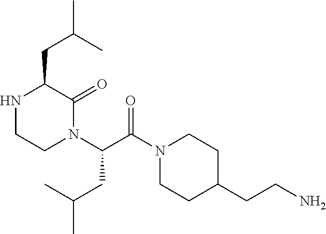 [SMILES: CC(C)C[C@@H]1NCCN([C@@H](CC(C)C)C(=O)N2CCC(CCN)CC2)C1=O] | 138 | (S)-1-[(S)- 1-{[4-(2- Aminoethyl)-1- piperidyl]carbonyl}-3- methylbutyl]-3- isobutyl-2- piperazinone | (B1) IIIb-Z = CHCH₂CH₂NHBOC (Combi-Blocks), (B2) BOC deprotection | CD₃OD: δ 5.53 (m, 1H), 4.46 (t, 1H), 2.99 (m, 2H), 1.68 (m, 10H), 0.97 (m, 12H) | +++ |
| 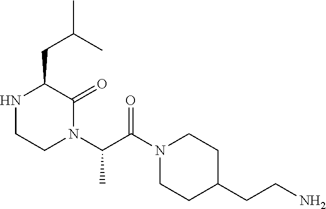 [SMILES: CC(C)C[C@@H]1NCCN([C@@H](C)C(=O)N2CCC(CCN)CC2)C1=O] | 139 | (S)-1-{(S)-2-[4-(2- Aminoethyl)-1- piperidyl]-1- methyl-2- oxoethyl}-3- isobutyl-2- piperazinone | (B1) IIIb-Z = CHCH₂CH₂NHBOC (Combi-Blocks), (B2) BOC deprotection | CD₃OD: δ 4.80 (m, 1H), 3.84 (d, 1H), 2.36 (m, 2H), 0.75 (d, 3H), 0.36 (m, 6H) | +* |
| 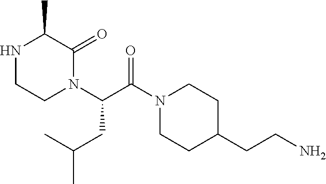 [SMILES: CC(C)C[C@@H](C(=O)N1CCC(CCN)CC1)N1CCN[C@@H](C)C1=O] | 140 | (S)-1-[(S)-1-{[4-(2- Aminoethyl)-1- piperidyl]carbonyl}-3- methylbutyl]- 3-methyl- 2-piperazinone | (B1) IIIb-Z = CHCH₂CH₂NHBOC (Combi-Blocks), (B2) BOC deprotection | CD₃OD: δ 4.77 (m, 1H), 3.76 (d, 1H), 2.27 (m, 2H), 0.88 (m, 3H), 0.24 (m, 6H) | +* |
| 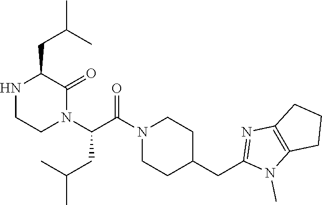 [SMILES: CC(C)C[C@@H]1NCCN([C@@H](CC(C)C)C(=O)N2CCC(Cc3nc4c(n3C)CCC4)CC2)C1=O] | 141 | (S)-1-[(S)-3- Methyl-1- {[4-({4-methyl-2.4- diazabicyclo[3.3.0]octa- 1(5),2-dien-3- yl}methyl)-1- piperidyl]car- bonyl}butyl]-3- isobutyl-2- piperazinone | Z = CHCH₂COOH (Combi-Blocks), amide coupling with 2-aminocyclo- pentanol, DMP oxidation to ketone, imine formation with methylamine using Ti(OiPr)₄, cyclization to imidazole by treatment with PCl₅ | CD₃OD: δ 5.55 (m, 1H), 4.47 (t, 1H), 3.55 (s, 3H), 2.65 (m, 6H), 2.49 (m, 2H), 0.95 (m, 12H) | +++ |
| 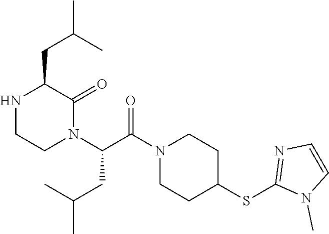 [SMILES: CC(C)C[C@@H]1NCCN([C@@H](CC(C)C)C(=O)N2CCC(Sc3nccn3C)CC2)C1=O] | 142 | (S)-1-[(S)-3-Methyl-1- {[4-(1-methyl-1H- imidazol-2-ylthio)-1- piperidyl]car- bonyl}butyl]-3- isobutyl-2- piperazinone | Z = CHOH, Mitsunobu reaction with 1- methyl-1H- imidazole-2-thiol | CD₃OD: δ 7.25 (s, 1H), 7.06 (s, 1H), 5.53 (m, 1H), 3.75 (s, 3H), 0.95 (m, 12H) | ++ |
| 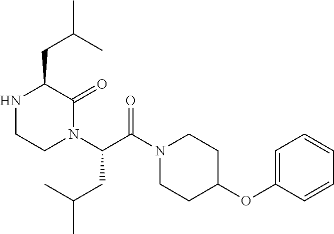 [SMILES: CC(C)C[C@@H]1NCCN([C@@H](CC(C)C)C(=O)N2CCC(Oc3ccccc3)CC2)C1=O] | 143 | (S)-1-{(S)-3-Methyl-1- [(4-phenoxy-1- piperidyl)car- bonyl]butyl}-3- isobutyl-2- piperazinone | Z = CHOH, Mitsunobu reaction with phenol | CD₃OD: δ 7.27 (t, 2H), 7.01- 6.88 (br m, 3H), 5.59 (m, 1H), 4.63 (m, 1H), 2.93 (m, 1H), 0.95 (m, 12H) | ++ |
| 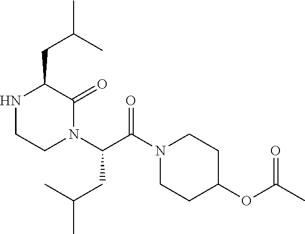 [SMILES: CC(=O)OC1CCN(C(=O)[C@H](CC(C)C)N2CCN[C@@H](CC(C)C)C2=O)CC1] | 144 | 1-{(S)-2-[(S)-3-Isobutyl- 2-oxo-1-piperazinyl]-4- methylvaleryl}-4- piperidyl acetate | Z = CHOH, acylation with acyl chloride | CD₃OD: δ 5.57 (m, 1H), 4.99 (m, 1H), 2.91 (m, 1H), 2.05 (d, 3H), 0.95 (m, 12H) | +* |
| 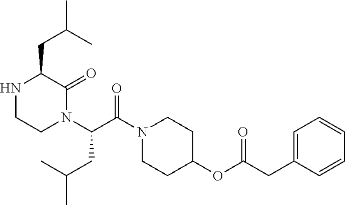 [SMILES: CC(C)C[C@@H]1NCCN([C@@H](CC(C)C)C(=O)N2CCC(OC(=O)Cc3ccccc3)CC2)C1=O] | 145 | 1-{(S)-2-[(S)-3-Isobutyl- 2-oxo-1-piperazinyl]-4- methylvaleryl}-4- piperidyl phenylacetate | Z = CHOH, acylation with phenylacetyl chloride | CD₃OD: δ 7.40-7.21 (br m, 5H), 5.55 (m, 1H), 5.00 (m, 1H), 3.65 (d, 2H), 2.90 (m, 1H), 0.95 (m, 12H) | +* |
| 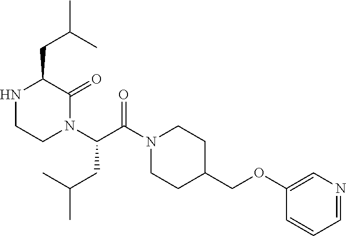 [SMILES: CC(C)C[C@@H]1NCCN([C@@H](CC(C)C)C(=O)N2CCC(COc3cccnc3)CC2)C1=O] | 146 | (S)-1-[(S)-3-Methyl-1- ({4-[(3- pyridyloxy)methyl]-1- piperidyl}car- bonyl)butyl]-3- isobutyl-2- piperazinone | Z = CHCH₂OH, Mitsunobu reaction with 3- Pyridinol | CD₃OD: δ 8.23 (d, 1H), 8.12 (dd, 1H), 7.43 (d, 1H), 7.35 (dd, 1H), 5.59 (m, 1H), 3.95 (d, 2H), 0.95 (m, 12H) | +* |
| 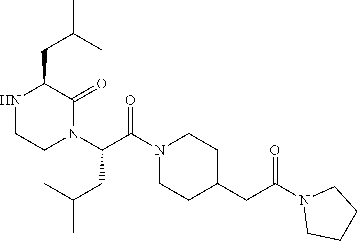 [SMILES: CC(C)C[C@@H]1NCCN([C@@H](CC(C)C)C(=O)N2CCC(CC(=O)N3CCCC3)CC2)C1=O] | 147 | (S)-1-[(S)-3-Methyl-1- ({4-[2-oxo-2-(1- pyrrolidinyl)ethyl]-1- piperidyl}car- bonyl)butyl]-3- isobutyl-2- piperazinone | (B1) IIIb-Z = CHCH₂C(O)OCH₃ (Combi-Blocks), (B2) hydrolysis, amide coupling | CD₃OD: δ 5.57 (m, 1H), 3.51 (t, 2H), 2.31 (m, 2H), 0.97 (m, 12H) | ++ |
| 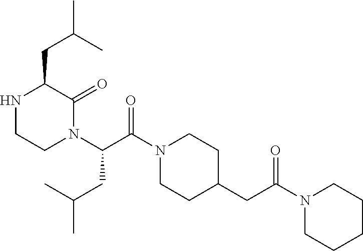 [SMILES: CC(C)C[C@@H]1NCCN([C@@H](CC(C)C)C(=O)N2CCC(CC(=O)N3CCCCC3)CC2)C1=O] | 148 | (S)-1-[(S)-3-Methyl-1- {[4-(2-oxo-2- piperidinoethyl)-1- piperidyl]car- bonyl}butyl]-3- isobutyl-2- piperazinone | (B1) IIIb-Z = CHCH₂C(O)OCH₃ (Combi-Blocks), (B2) hydrolysis, amide coupling | CD₃OD: δ 5.57 (m, 1H), 3.54 (m, 4H), 2.36 (m, 2H), 0.97 (m, 12H) | ND |
| 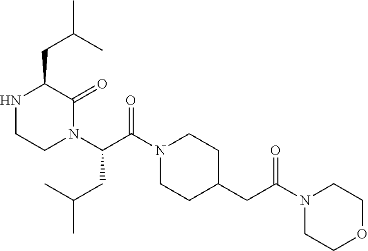 [SMILES: CC(C)C[C@@H]1NCCN([C@@H](CC(C)C)C(=O)N2CCC(CC(=O)N3CCOCC3)CC2)C1=O] | 149 | (S)-1-[(S)-3-Methyl-1- {[4-(2-morpholino-2- oxoethyl)-1- piperidyl]car- bonyl}butyl]-3- isobutyl-2- piperazinone | (B1) IIIb-Z = CHCH₂C(O)OCH₃ (Combi-Blocks), (B2) hydrolysis, amide coupling | CD₃OD: δ 5.57 (m, 1H), 3.66 (m, 4H), 2.37 (m, 2H), 0.97 (m, 12H) | +* |
| 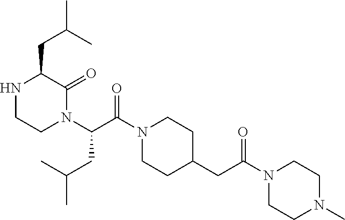 [SMILES: CC(C)C[C@@H]1NCCN([C@@H](CC(C)C)C(=O)N2CCC(CC(=O)N3CCN(C)CC3)CC2)C1=O] | 150 | (S)-1-[(S)-3-Methyl-1- ({4-[2-(4-methyl-1- piperazinyl)-2- oxoethyl]-1- piperidyl}car- bonyl)butyl]-3- isobutyl-2- piperazinone | (B1) IIIb-Z = CHCH₂C(O)OCH₃ (Combi-Blocks), (B2) hydrolysis, amide coupling | CD₃OD: δ 5.57 (m, 1H), 3.61 (m, 4H), 2.33 (s, 3H), 0.97 (m, 12H) | ++ |
| 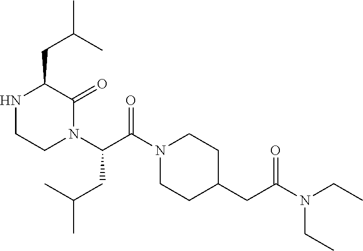 [SMILES: CCN(CC)C(=O)CC1CCN(C(=O)[C@H](CC(C)C)N2CCN[C@@H](CC(C)C)C2=O)CC1] | 151 | (S)-1-[(S)-1-({4-[2- (Diethylamino)-2- oxoethyl]-1- piperidyl}carbonyl)-3- methylbutyl]-3- isobutyl-2-piperazinone | (B1) IIIb-Z = CHCH₂C(O)OCH₃ (Combi-Blocks), (B2) hydrolysis, amide coupling | CD₃OD: δ 5.57 (m, 1H), 3.41 (m, 6H), 2.33 (m, 2H), 0.97 (m, 12H) | +* |
| 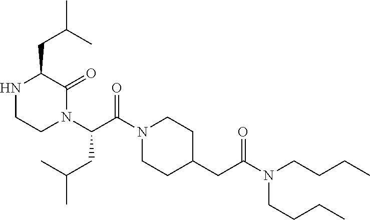 [SMILES: CCCCN(CCCC)C(=O)CC1CCN(C(=O)[C@H](CC(C)C)N2CCN[C@@H](CC(C)C)C2=O)CC1] | 152 | (S)-1-[(S)-1-({4-[2- (Dibutylamino)-2- oxoethyl]-1- piperidyl}carbonyl)-3- methylbutyl]-3- isobutyl-2-piperazinone | (B1) IIIb-Z = CHCH₂C(O)OCH₃ (Combi-Blocks), (B2) hydrolysis, amide coupling | CD₃OD: δ 5.57 (m, 1H), 2.33 (m, 2H), 1.36 (m, 6H), 0.98 (m, 12H) | +++ |
| 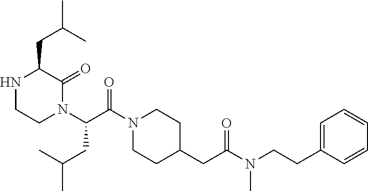 [SMILES: CC(C)C[C@@H]1NCCN([C@@H](CC(C)C)C(=O)N2CCC(CC(=O)N(C)CCc3ccccc3)CC2)C1=O] | 153 | (S)-1-[(S)-1-[(4-{2-[N- Methyl(phenethyl)a- mino]-2-oxoethyl}-1- piperidyl)carbonyl]-3- methylbutyl]-3- isobutyl-2-piperazinone | (B1) IIIb-Z = CHCH₂C(O)OCH₃ (Combi-Blocks), (B2) hydrolysis, amide coupling | CD₃OD: δ 7.34-7.18 (m, 5H), 5.55 (m, 1H), 3.63 (m, 2H), 0.97 (m, 12H) | ++ |
| 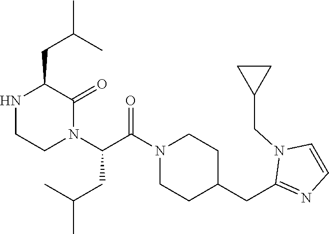 [SMILES: CC(C)C[C@@H]1NCCN([C@@H](CC(C)C)C(=O)N2CCC(Cc3nccn3CC3CC3)CC2)C1=O] | 154 | (S)-1-[(S)-1-[(4-{[1- (Cyclopropylmethyl)- 1H-imidazol-2- yl]methyl}-1- piperidyl)carbonyl]-3- methylbutyl]-3- isobutyl-2-piperazinone | B1) IIIb- Z = CHCH₂(1H- imidazole) (Enamine BB) (B2) N-alkylation | CD₃OD: δ, 6.77 (d, 2H), 5.56 (q, 1H), 4.57 (t, 1H), 0.9 (m, 12H), 0.67 (m 2H), 0.33 (m, 2H). | ++ |
| 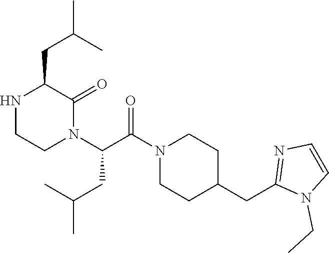 [SMILES: CCn1ccnc1CC1CCN(C(=O)[C@H](CC(C)C)N2CCN[C@@H](CC(C)C)C2=O)CC1] | 155 | (S)-1-[(S)-1-({4-[(1- Ethyl-1H-imidazol-2- yl)methyl]-1- piperidyl}carbonyl)-3- methylbutyl]-3- isobutyl-2-piperazinone | B1) IIIb- Z = CHCH₂(1H- imidazole) (Enamine BB) (B2) N-alkylation | CD₃OD: δ, 6.95 (S, 1H), 6.85 (s, 1H), 5.56 (q, 1H), 4.50 (t, 1H), 0.95, (m, 15H). | +++ |
| 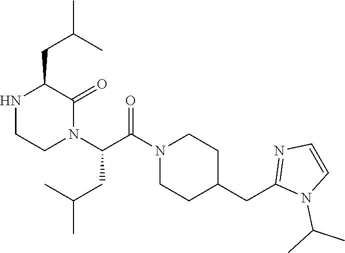 [SMILES: CC(C)C[C@@H]1NCCN([C@@H](CC(C)C)C(=O)N2CCC(Cc3nccn3C(C)C)CC2)C1=O] | 156 | (S)-1-[(S)-1-({4-[(1- Isopropyl-1H-imidazol- 2-yl)methyl]-1- piperidyl}carbonyl)-3- methylbutyl]-3- isobutyl-2-piperazinone | B1) IIIb- Z = CHCH₂(1H- imidazole) (Enamine BB) (B2) N-alkylation | CD₃OD: δ, 7.70 (s, 1H), 7.50 (s, 1H), 5.56 (q, 1H), 4.50 (t, 1H), 1.5 (d, 6H), 0.95 (m, 12H). | +++ |
| 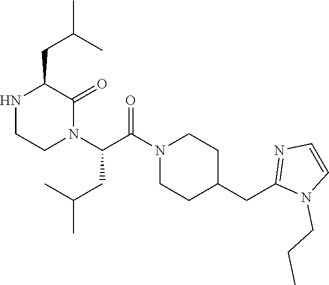 [SMILES: CCCn1ccnc1CC1CCN(C(=O)[C@H](CC(C)C)N2CCN[C@@H](CC(C)C)C2=O)CC1] | 157 | (S)-1-[(S)-3-Methyl-1- ({4-[(1-propyl-1H- imidazol-2- yl)methyl]-1- piperidyl}car- bonyl)butyl]-3- isobutyl-2- piperazinone | B1) IIIb- Z = CHCH₂(1H- imidazole) (Enamine BB) (B2) N-alkylation | CD₃OD: δ 6.96 (S, 1H), 6.86 (s, 1H), 5.55 (q, 1H)), 4.52 (t, 1H), 0.95, (m, 15H). | +++ |
| 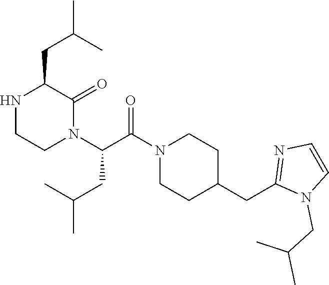 [SMILES: CC(C)C[C@@H]1NCCN([C@@H](CC(C)C)C(=O)N2CCC(Cc3nccn3CC(C)C)CC2)C1=O] | 158 | (S)-1-[(S)-1-({4-[(1- Isobutyl-1H- imidazol-2- yl)methyl]-1- piperidyl}carbonyl)-3- methylbutyl]-3- isobutyl-2-piperazinone | B1) IIIb- Z = CHCH₂(1H- imidazole) (Enamine BB) (B2) N-alkylation | CD₃OD: δ 7.08 (S, 1H), 6.86 (s, 1H), 5.60 (q, 1H), 4.50 (t, 1H), 0.90, (m, 18H). | +++ |
| 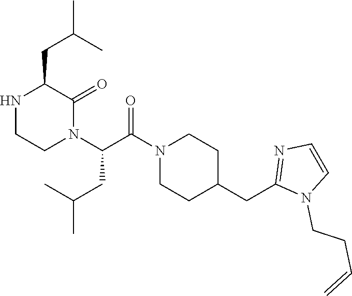 [SMILES: C=CCCn1ccnc1CC1CCN(C(=O)[C@H](CC(C)C)N2CCN[C@@H](CC(C)C)C2=O)CC1] | 159 | (S)-1-[(S)-1-[(4-{[1-(3- Butenyl)-1H-imidazol-2- yl]methyl}-1- piperidyl)carbonyl]-3- methylbutyl]-3- isobutyl-2-piperazinone | B1) IIIb- Z = CHCH₂(1H- imidazole) (Enamine BB) (B2) N-alkylation | CD₃OD: δ, 6.80 (m (1H), 6.75 (s, 1H), 5.56 (q, 1H) 4.45 (t, 1H), 0.94, (m, 12H). | +++ |
| 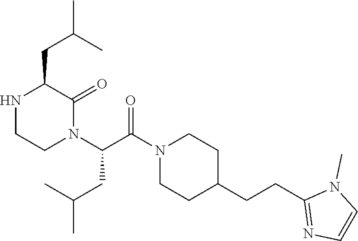 [SMILES: CC(C)C[C@@H]1NCCN([C@@H](CC(C)C)C(=O)N2CCC(CCc3nccn3C)CC2)C1=O] | 160 | (S)-1-[(S)-3-Methyl-1- ({4-[2-(1-methyl-1H- imidazol-2-yl)ethyl]-1- piperidyl}car- bonyl)butyl]-3- isobutyl-2- piperazinone | B1) IIIb- Z = CHCH₂CH₂OH, (B2) oxidation to aldehyde, N- methyl imidazole construction (with ammonia, methylamine and glyoxal), tBOC removal | CDCl3: δ, 6.90 (S, 1H), 6.78 (s, 1H), 5.60 (q, 1H), 4.50 (t, 1H), 3.55 (s, 3H), 0.95, (m, 13H). | +++ |
| 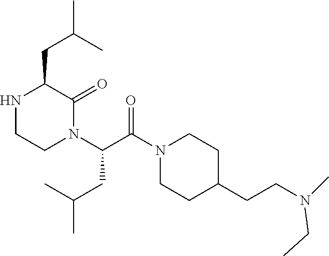 [SMILES: CCN(C)CCC1CCN(C(=O)[C@H](CC(C)C)N2CCN[C@@H](CC(C)C)C2=O)CC1] | 161 | (S)-1-[(S)-1-({4-[2-(N- Methyl-N- ethylamino)ethyl]-1- piperidyl}carbonyl)-3- methylbutyl]-3- isobutyl-2-piperazinone | Z = CHCH₂CH₂OH, oxidation to aldehyde, reductive amination | ¹H NMR (CD₃OD): δ 0.98 (m, 12H), 1.23 (m, 5H), 1.71 (m, 10H), 2.86 (m, 4H), 4.48 (m, 1H), 5.55 (m, 1H) | ++++ |
| 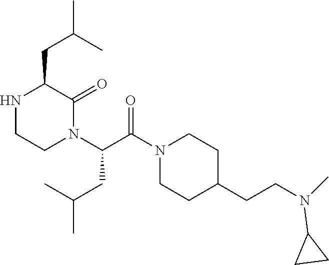 [SMILES: CC(C)C[C@@H]1NCCN([C@@H](CC(C)C)C(=O)N2CCC(CCN(C)C3CC3)CC2)C1=O] | 162 | (S)-1-[(S)-1-({4-[2-(N- Methyl-N- cyclopropylamino)ethyl]- 1-piperidyl}carbonyl)- 3-methylbutyl]-3- isobutyl-2-piperazinone | Z = CHCH₂CH₂OH, oxidation to aldehyde, reductive amination | ¹H NMR CD₃OD: δ 0.48 (m, 2H), 0.97 (m, 12H), 1.09 (m, 2H), 1.64 (m, 12H), 2.36 (s, 3H), 2.62 (m, 3H), 5.57 (m, 1H) | ++++ |
| 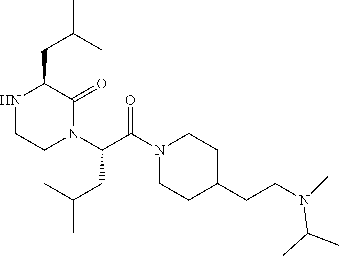 [SMILES: CC(C)C[C@@H]1NCCN([C@@H](CC(C)C)C(=O)N2CCC(CCN(C)C(C)C)CC2)C1=O] | 163 | (S)-1-[(S)-1-[(4-{2-[N- Methyl(isopropyl)a- mino]ethyl}-1- piperidyl)carbonyl]-3- methylbutyl]-3- isobutyl-2-piperazinone | Z = CHCH₂CH₂OH, oxidation to aldehyde, reductive amination | ¹H NMR CD₃OD: δ 0.96 (m, 12H), 1.15 (m, 5H), 2.34 (m, 2H), 4.08 (m, 1H), 4.49 (m, 1H), 5.56 (m, 1H) | ++ |
| 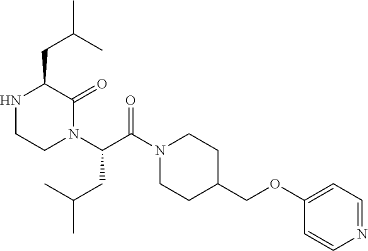 [SMILES: CC(C)C[C@@H]1NCCN([C@@H](CC(C)C)C(=O)N2CCC(COc3ccncc3)CC2)C1=O] | 164 | (S)-1-[(S)-3-Methyl-1- ({4-[(4- pyridyloxy)methyl]-1- piperidyl}car- bonyl)butyl]-3- isobutyl-2- piperazinone | Z = CHCH₂OH, Mitsunobu reaction with 4- Pyridinol | CD₃OD: δ 8.33 (d, 2H), 6.99 (d, 2H), 5.58 (m, 1H), 3.98 (d, 2H), 0.95 (m, 12H) | +++ |
| 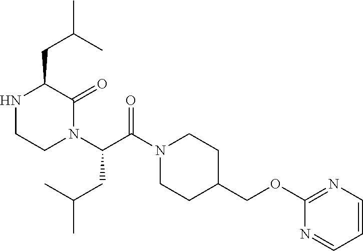 [SMILES: CC(C)C[C@@H]1NCCN([C@@H](CC(C)C)C(=O)N2CCC(COc3ncccn3)CC2)C1=O] | 165 | (S)-1-[(S)-3-Methyl-1- ({4-[(2- pyrimidinyloxy)methyl]- 1- piperidyl}car- bonyl)butyl]-3- isobutyl-2- piperazinone | Z = CHCH₂OH, Mitsunobu reaction with methyl 2- hydroxypyrimi- dine | CD₃OD: δ 8.55 (d, 2H), 7.09 (t, 1H), 5.58 (m, 1H), 4.28 (d, 2H), 0.95 (m, 12H) | +* |
| 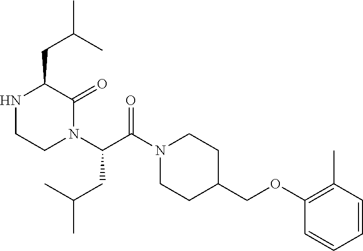 [SMILES: Cc1ccccc1OCC1CCN(C(=O)[C@H](CC(C)C)N2CCN[C@@H](CC(C)C)C2=O)CC1] | 166 | (S)-1-[(S)-3-Methyl-1- ({4-[(o- tolyloxy)methyl]-1- piperidyl}car- bonyl)butyl]-3- isobutyl-2- piperazinone | Z = CHCH₂OH, Mitsunobu reaction with o- cresol | CD₃OD: δ 7.17-7.03 (br m, 2H), 6.90-6.74 (br m, 2H), 5.59 (m, 1H), 3.85 (d, 2H), 2.18 (s, 3H), 0.95 (m, 12H) | +++ |
| 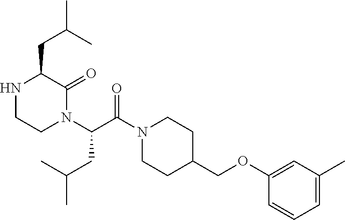 [SMILES: Cc1cccc(OCC2CCN(C(=O)[C@H](CC(C)C)N3CCN[C@@H](CC(C)C)C3=O)CC2)c1] | 167 | (S)-1-[(S)-3-Methyl-1- ({4-[(m- tolyloxy)methyl]-1- piperidyl}car- bonyl)butyl]-3- isobutyl-2- piperazinone | Z = CHCH₂OH, Mitsunobu reaction with m- cresol | CD₃OD: δ 7.11 (t, 1H), 6.77- 6.60 (br m, 3H), 5.58 (m, 1H), 3.82 (d, 2H), 2.29 (s, 3H), 0.95 (m, 12H) | +++ |
| 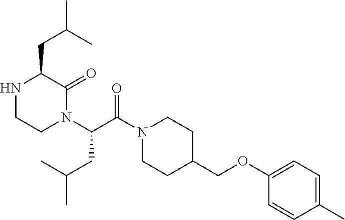 [SMILES: Cc1ccc(OCC2CCN(C(=O)[C@H](CC(C)C)N3CCN[C@@H](CC(C)C)C3=O)CC2)cc1] | 168 | (S)-1-[(S)-3-Methyl-1- ({4-[(p- tolyloxy)methyl]-1- piperidyl}car- bonyl)butyl]-3- isobutyl-2- piperazinone | Z = CHCH₂OH, Mitsunobu reaction with p- cresol | CD₃OD: δ 7.05 (d, 2H), 6.78 (d, 2H), 5.58 (m, 1H), 3.80 (d, 2H), 2.24 (s, 3H), 0.95 (m, 12H) | +++ |
| 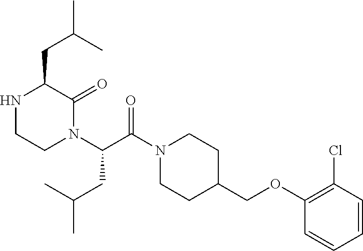 [SMILES: CC(C)C[C@@H]1NCCN([C@@H](CC(C)C)C(=O)N2CCC(COc3ccccc3Cl)CC2)C1=O] | 169 | (S)-1-[(S)-1-({4-[(o- Chlorophenoxy)methyl]- 1-piperidyl}carbonyl)- 3-methylbutyl]-3- isobutyl-2-piperazinone | Z = CHCH₂OH, Mitsunobu reaction with o- chlorophenol | CD₃OD: δ 7.34 (d, 1H), 7.24 (t, 1H), 7.04 (d, 1H), 6.90 (t, 1H), 5.60 (m, 1H), 4.00- 3.87 (m, 2H), 0.96 (m, 12H) | +++ |
| 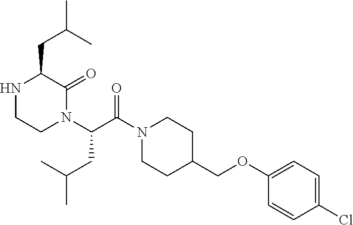 [SMILES: CC(C)C[C@@H]1NCCN([C@@H](CC(C)C)C(=O)N2CCC(COc3ccc(Cl)cc3)CC2)C1=O] | 170 | (S)-1-[(S)-1-({4-[(p- Chlorophenoxy)methyl]- 1-piperidyl}carbonyl)- 3-methylbutyl]-3- isobutyl-2-piperazinone | Z = CHCH₂OH, Mitsunobu reaction with p- chlorophenol | CD₃OD: δ 7.23 (d, 2H), 6.89 (d, 2H), 5.59 (m, 1H), 3.84 (d, 2H), 0.95 (m, 12H) | +++ |
| 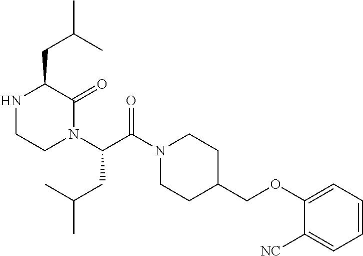 [SMILES: CC(C)C[C@@H]1NCCN([C@@H](CC(C)C)C(=O)N2CCC(COc3ccccc3C#N)CC2)C1=O] | 171 | o-[(1-{(S)-2-[(S)-3- Isobutyl-2-oxo-1- piperazinyl]-4- methylvaleryl}-4- piperidyl)meth- oxy]benzonitrile | Z = CHCH₂OH, Mitsunobu reaction with salicylonitrile | CD₃OD: δ 7.66-7.50 (br m, 2H), 7.15 (d, 1H), 7.05 (t, 1H), 5.59 (m, 1H), 4.02 (d, 2H), 0.94 (m, 12H) | +++ |
| 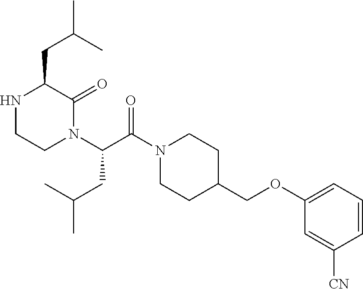 [SMILES: CC(C)C[C@@H]1NCCN([C@@H](CC(C)C)C(=O)N2CCC(COc3cccc(C#N)c3)CC2)C1=O] | 172 | m-[(1-{(S)-2-[(S)-3- Isobutyl-2-oxo-1- piperazinyl]-4- methylvaleryl}-4- piperidyl)meth- oxy]benzonitrile | Z = CHCH₂OH, Mitsunobu reaction with m- hydroxybenzo- nitrile | CD₃OD: δ 7.44 (t, 1H), 7.33- 7.19 (br m, 3H), 5.59 (m, 1H), 3.91 (d, 2H), 0.95 (m, 12H) | +++ |
| 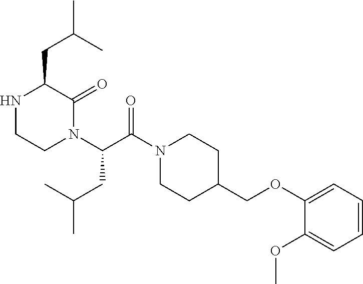 [SMILES: COc1ccccc1OCC1CCN(C(=O)[C@H](CC(C)C)N2CCN[C@@H](CC(C)C)C2=O)CC1] | 173 | (S)-1-[(S)-1-({4-[(o- Methoxy- phenoxy)methyl]-1- piperidyl}carbonyl)-3- methylbutyl]-3- isobutyl-2-piperazinone | Z = CHCH₂OH, Mitsunobu reaction with guaiacol | CD₃OD: δ 6.98-6.78 (br m, 4H), 5.58 (m, 1H), 3.94- 3.83 (br m, 2H), 3.81 (s, 3H), 0.95 (m, 12H) | ++ |
| 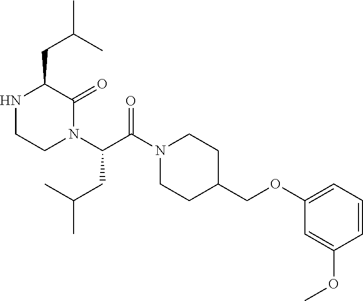 [SMILES: COc1cccc(OCC2CCN(C(=O)[C@H](CC(C)C)N3CCN[C@@H](CC(C)C)C3=O)CC2)c1] | 174 | (S)-1-[(S)-1-({4-[(m- Methoxy- phenoxy)methyl]-1- piperidyl}carbonyl)-3- methylbutyl]-3- isobutyl-2-piperazinone | Z = CHCH₂OH, Mitsunobu reaction with m- methoxyphenol | CD₃OD: δ 7.14 (t, 1H), 6.54- 6.42 (br m, 3H), 5.59 (m, 1H), 3.82 (d, 2H), 3.75 (s, 3H), 0.95 (m, 12H) | +++ |
| 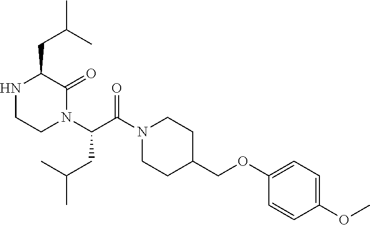 [SMILES: COc1ccc(OCC2CCN(C(=O)[C@H](CC(C)C)N3CCN[C@@H](CC(C)C)C3=O)CC2)cc1] | 175 | (S)-1-[(S)-1-({4-[(p- Methoxy- phenoxy)methyl]-1- piperidyl}carbonyl)-3- methylbutyl]-3- isobutyl-2-piperazinone | Z = CHCH₂OH, Mitsunobu reaction with p- methoxyphenol | CD₃OD: δ 6.91-6.77 (br m, 4H), 5.58 (m, 1H), 3.78 (d, 2H), 3.73 (s, 3H), 0.95 (m, 12H) | +++ |
| 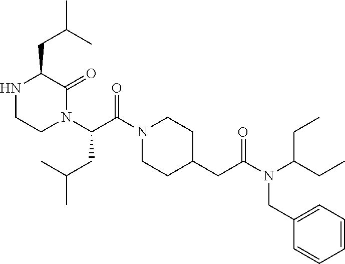 [SMILES: CCC(CC)N(Cc1ccccc1)C(=O)CC1CCN(C(=O)[C@H](CC(C)C)N2CCN[C@@H](CC(C)C)C2=O)CC1] | 176 | (S)-1-[(S)-1-[(4-{2- [(Benzyl)(1- ethylpropyl)amino]-2- oxoethyl}-1- piperidyl)carbonyl]-3- methylbutyl]-3- isobutyl-2-piperazinone | (B1) IIIb-Z = CHCH₂C(O)OCH₃ (Combi-Blocks), (B2) hydrolysis, amide coupling | CD₃OD: δ 7.41-7.18 (m, 5H), 5.57 (m, 1H), 0.97 (m, 12H), 0.90-0.78 (m, 6H) | +++ |
| 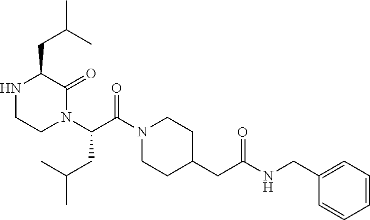 [SMILES: CC(C)C[C@@H]1NCCN([C@@H](CC(C)C)C(=O)N2CCC(CC(=O)NCc3ccccc3)CC2)C1=O] | 177 | (S)-1-[(S)-1-{[4-(2- Benzylamino-2- oxoethyl)-1- piperidyl]carbonyl}-3- methylbutyl]-3- isobutyl-2-piperazinone | (B1) IIIb-Z = CHCH₂C(O)OCH₃ (Combi-Blocks), (B2) hydrolysis, amide coupling | CD₃OD: δ 5.57 (m, 1H), 4.38 (s, 2H), 2.20 (m, 2H), 0.96 (m, 12H) | ++ |
| 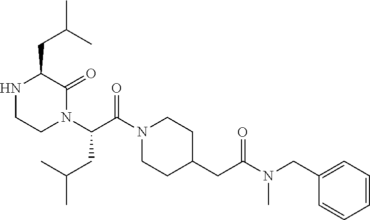 [SMILES: CC(C)C[C@@H]1NCCN([C@@H](CC(C)C)C(=O)N2CCC(CC(=O)N(C)Cc3ccccc3)CC2)C1=O] | 178 | (S)-1-[(S)-1-[(4-{2-[N- Methyl(benzyl)amino]- 2-oxoethyl}-1- piperidyl)carbonyl]-3- methylbutyl]-3- isobutyl-2-piperazinone | (B1) IIIb-Z = CHCH₂C(O)OCH₃ (Combi-Blocks), (B2) hydrolysis, amide coupling | CD₃OD: δ 5.57 (m, 1H), 4.64 (m, 2H), 2.42 (m, 2H), 0.96 (m, 12H) | +++ |
| 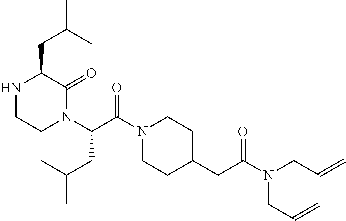 [SMILES: C=CCN(CC=C)C(=O)CC1CCN(C(=O)[C@H](CC(C)C)N2CCN[C@@H](CC(C)C)C2=O)CC1] | 179 | (S)-1-[(S)-1-({4-[2- (Diallylamino)-2- oxoethyl]-1- piperidyl}carbonyl)-3- methylbutyl]-3- isobutyl-2-piperazinone | (B1) IIIb-Z = CHCH₂C(O)OCH₃ (Combi-Blocks), (B2) hydrolysis, amide coupling | CD₃OD: δ 5.93-5.72 (m, 2H), 5.57 (m, 1H), 2.35 (m, 2H), 0.97 (m, 12H) | ++ |
| 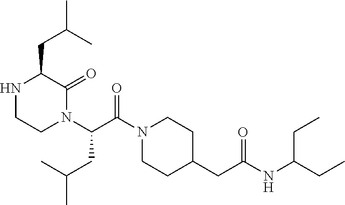 [SMILES: CCC(CC)NC(=O)CC1CCN(C(=O)[C@H](CC(C)C)N2CCN[C@@H](CC(C)C)C2=O)CC1] | 180 | (S)-1-[(S)-1-({4-[2-(1- Ethylpropylamino)-2- oxoethyl]-1- piperidyl}carbonyl)-3- methylbutyl]-3- isobutyl-2-piperazinone | (B1) IIIb-Z = CHCH₂C(O)OCH₃ (Combi-Blocks), (B2) hydrolysis, amide coupling | CD₃OD: δ 5.58 (m, 1H), 3.67 (m, 1H), 2.17 (m, 2H), 1.01- 0.87 (m, 18H) | ++ |
| 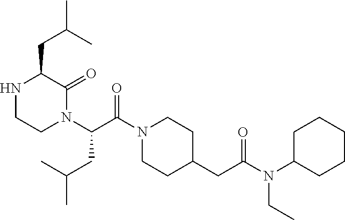 [SMILES: CCN(C(=O)CC1CCN(C(=O)[C@H](CC(C)C)N2CCN[C@@H](CC(C)C)C2=O)CC1)C1CCCCC1] | 181 | (S)-1-[(S)-1-({4-[2-(N- Ethyl-N- cyclohexylamino)-2- oxoethyl]-1- piperidyl}carbonyl)-3- methylbutyl]-3- isobutyl-2-piperazinone | (B1) IIIb-Z = CHCH₂C(O)OCH₃ (Combi-Blocks), (B2) hydrolysis, amide coupling | CD₃OD: δ 5.56 (m, 1H), 2.35 (m, 2H), 1.23-1.10 (m, 5H), 0.96 (m, 12H) | ++ |
| 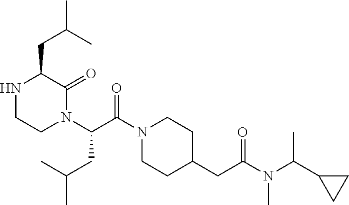 [SMILES: CC(C)C[C@@H]1NCCN([C@@H](CC(C)C)C(=O)N2CCC(CC(=O)N(C)C(C)C3CC3)CC2)C1=O] | 182 | (S)-1-[(S)-1-[(4-{2-[N- Methyl(1-cyclopropyl- ethyl)amino]-2- oxoethyl}-1- piperidyl)carbonyl]-3- methylbutyl]-3- isobutyl-2-piperazinone | (B1) IIIb-Z = CHCH₂C(O)OCH₃ (Combi-Blocks), (B2) hydrolysis, amide coupling | CD₃OD: δ 5.57 (m, 1H), 2.39- 2.26 (m, 2H), 0.96 (m, 12H), 0.24-0.12 (m, 1H) | +* |
| 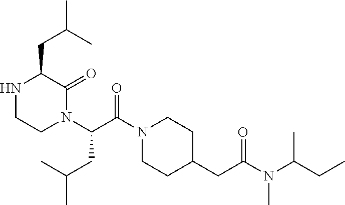 [SMILES: CCC(C)N(C)C(=O)CC1CCN(C(=O)[C@H](CC(C)C)N2CCN[C@@H](CC(C)C)C2=O)CC1] | 183 | (S)-1-[(S)-1-[(4-{2-[N- Methyl(sec- butyl)amino]-2- oxoethyl}-1- piperidyl)carbonyl]-3- methylbutyl]-3- isobutyl-2-piperazinone | (B1) IIIb-Z = CHCH₂C(O)OCH₃ (Combi-Blocks), (B2) hydrolysis, amide coupling | CD₃OD: δ 5.57 (m, 1H), 2.36 (m, 2H), 0.97 (m, 12H), 0.92-0.81 (m, 3H) | +* |
| 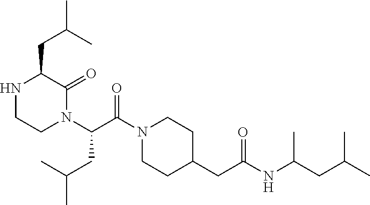 [SMILES: CC(C)CC(C)NC(=O)CC1CCN(C(=O)[C@H](CC(C)C)N2CCN[C@@H](CC(C)C)C2=O)CC1] | 184 | (S)-1-[(S)-1-({4-[2-(1,3- Dimethylbutylamino)-2- oxoethyl]-1- piperidyl}carbonyl)-3- methylbutyl]-3- isobutyl-2-piperazinone | (B1) IIIb-Z = CHCH₂C(O)OCH₃ (Combi-Blocks), (B2) hydrolysis, amide coupling | CD₃OD: δ 5.56 (m, 1H), 4.01 (m, 1H), 2.11 (m, 2H), 0.95 (m, 18H) | ++ |
| 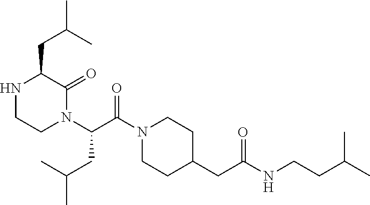 [SMILES: CC(C)CCNC(=O)CC1CCN(C(=O)[C@H](CC(C)C)N2CCN[C@@H](CC(C)C)C2=O)CC1] | 185 | (S)-1-[(S)-1-({4-[2- (Isopentylamino)-2- oxoethyl]-1- piperidyl}carbonyl)-3- methylbutyl]-3- isobutyl-2-piperazinone | (B1) IIIb-Z = CHCH₂C(O)OCH₃ (Combi-Blocks), (B2) hydrolysis, amide coupling | CD₃OD: δ 5.57 (m, 1H), 3.21 (t, 2H), 2.13 (m, 2H), 0.96 (m, 18H) | ++ |
| 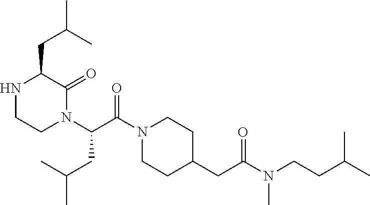 [SMILES: CC(C)CCN(C)C(=O)CC1CCN(C(=O)[C@H](CC(C)C)N2CCN[C@@H](CC(C)C)C2=O)CC1] | 186 | (S)-1-[(S)-1-[(4-{2-[N- Methyl(isopentyl)a- mino]-2-oxoethyl}-1- piperidyl)carbonyl]-3- methylbutyl]-3- isobutyl-2-piperazinone | (B1) IIIb-Z = CHCH₂C(O)OCH₃ (Combi-Blocks), (B2) hydrolysis, amide coupling | CD₃OD: δ 5.58 (m, 1H), 3.40 (m, 4H), 2.34 (m, 2H), 0.97 (m, 18H) | ++ |
| 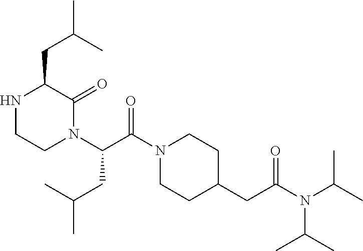 [SMILES: CC(C)C[C@@H]1NCCN([C@@H](CC(C)C)C(=O)N2CCC(CC(=O)N(C(C)C)C(C)C)CC2)C1=O] | 187 | (S)-1-[(S)-1-[(4-{2- [Bis(isopropyl)amino]- 2-oxoethyl}-1- piperidyl)carbonyl]-3- methylbutyl]-3- isobutyl-2-piperazinone | (B1) IIIb-Z = CHCH₂C(O)OCH₃ (Combi-Blocks), (B2) hydrolysis, amide coupling | CD₃OD: δ 5.57 (m, 1H), 2.31 (m, 2H), 1.39 (d, 6H), 0.96 (m, 12H) | ++ |
| 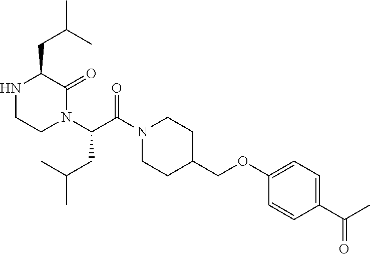 [SMILES: CC(=O)c1ccc(OCC2CCN(C(=O)[C@H](CC(C)C)N3CCN[C@@H](CC(C)C)C3=O)CC2)cc1] | 188 | (S)-1-[(S)-1-({4-[(p- Acetylphenoxy)methyl]- 1-piperidyl}carbonyl)-3- methylbutyl]-3- isobutyl-2-piperazinone | Z = CHCH₂OH, Mitsunobu reaction with 4′- hydroxyacetophenone | CD₃OD: δ 7.96 (d, 2H), 7.00 (d, 2H), 5.59 (m, 1H), 3.95 (d, 2H), 2.54 (s, 3H), 0.95 (m, 12H) | +++ |
| 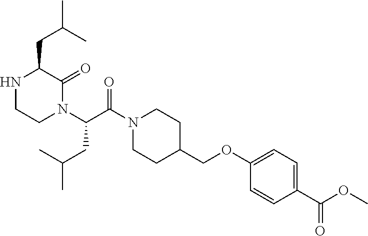 [SMILES: COC(=O)c1ccc(OCC2CCN(C(=O)[C@H](CC(C)C)N3CCN[C@@H](CC(C)C)C3=O)CC2)cc1] | 189 | Methyl p-[(1-{(S)- 2-[(S)- 3-isobutyl-2-oxo-1- piperazinyl]-4- methylvaleryl}-4- piperidyl)meth- oxy]benzoate | Z = CHCH₂OH, Mitsunobu reaction with methyl p- hydroxybenzoate | CD₃OD: δ 7.95 (d, 2H), 6.98 (d, 2H), 5.59 (m, 1H), 3.94 (d, 2H), 3.86 (s, 3H), 0.95 (m, 12H) | +++ |
| 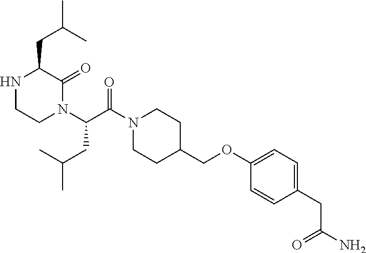 [SMILES: CC(C)C[C@@H]1NCCN([C@@H](CC(C)C)C(=O)N2CCC(COc3ccc(CC(N)=O)cc3)CC2)C1=O] | 190 | {p-[(1-{(S)-2-[(S)-3- Isobutyl-2-oxo-1- piperazinyl]-4- methylvaleryl}-4- piperidyl)meth- oxy]phenyl}acetamide | Z = CHCH₂OH, Mitsunobu reaction with 4- hydroxyphenyl- acetamide | CD₃OD: δ 7.20 (d, 2H), 6.86 (d, 2H), 5.58 (m, 1H), 3.84 (d, 2H), 3.43 (s, 2H), 0.95 (m, 12H) | ++ |
| 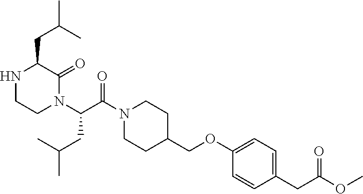 [SMILES: COC(=O)Cc1ccc(OCC2CCN(C(=O)[C@H](CC(C)C)N3CCN[C@@H](CC(C)C)C3=O)CC2)cc1] | 191 | Methyl {p-[(1-{(S)-2- [(S)-3-isobutyl-2-oxo-1- piperazinyl]-4- methylvaleryl}-4- piperidyl)meth- oxy]phenyl}acetate | Z = CHCH₂OH, Mitsunobu reaction with methyl 4- hydroxyphenyl- acetate | CD₃OD: δ 7.15 (d, 2H), 6.84 (d, 2H), 5.58 (m, 1H), 3.81 (d, 2H), 3.64 (s, 3H), 3.54 (s, 2H), 0.94 (m, 12H) | +++ |
| 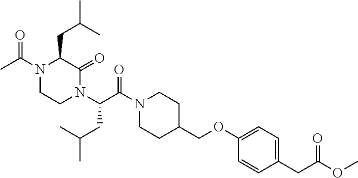 [SMILES: COC(=O)Cc1ccc(OCC2CCN(C(=O)[C@H](CC(C)C)N3CCN(C(C)=O)[C@@H](CC(C)C)C3=O)CC2)cc1] | 192 | Methyl {p-[(1-{(S)-2- [(S)-4-acetyl-3-isobutyl- 2-oxo-1-piperazinyl]-4- methylvaleryl}-4- piperidyl)meth- oxy]phenyl}acetate | Z = CHCH₂OH, Mitsunobu reaction with methyl 4- hydroxyphenyl- acetate | CDCl₃: δ 7.18 (d, 2H), 6.83 (d, 2H), 3.80 (d, 2H), 3.68 (s, 3H), 3.56 (s, 3H), 0.97 (m, 12H) | +++ |
| 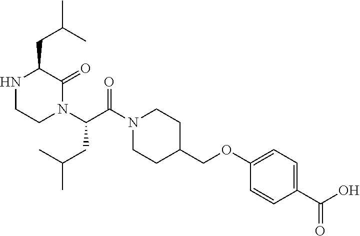 [SMILES: CC(C)C[C@@H]1NCCN([C@@H](CC(C)C)C(=O)N2CCC(COc3ccc(C(=O)O)cc3)CC2)C1=O] | 193 | p-[(1-{(S)-2-[(S)-3- Isobutyl-2-oxo-1- piperazinyl]-4- methylvaleryl}-4- piperidyl)meth- oxy]benzoic acid | Z = CHCH₂OH, Mitsunobu reaction with methyl p- hydroxybenzoate, ester hydrolysis | CD₃OD: δ 7.89 (d, 2H), 6.86 (d, 2H), 5.59 (m, 1H), 3.90 (d, 2H), 0.95 (m, 12H) | +* |
| 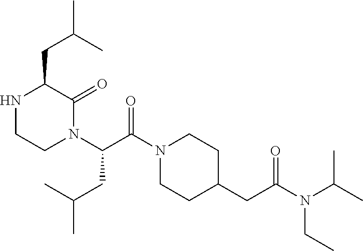 [SMILES: CCN(C(=O)CC1CCN(C(=O)[C@H](CC(C)C)N2CCN[C@@H](CC(C)C)C2=O)CC1)C(C)C] | 194 | (S)-1-[(S)-1-[(4-{2-[N- Ethyl(isopropyl)amino]- 2-oxoethyl}-1- piperidyl)carbonyl]-3- methylbutyl]-3- isobutyl-2-piperazinone | (B1) IIIb-Z = CHCH₂C(O)OCH₃ (Combi-Blocks), (B2) hydrolysis, amide coupling | CD₃OD: δ 5.57 (m, 1H), 2.35 (m, 2H), 1.26-1.11 (m, 11H), 0.97 (m, 12H) | ++ |
| 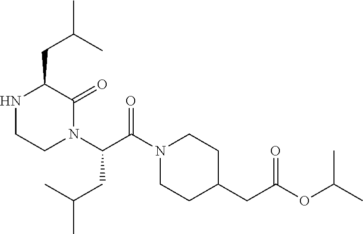 [SMILES: CC(C)C[C@@H]1NCCN([C@@H](CC(C)C)C(=O)N2CCC(CC(=O)OC(C)C)CC2)C1=O] | 195 | Isopropyl (1-{(S)- 2-[(S)- 3-isobutyl-2-oxo-1- piperazinyl]-4- methylvaleryl}-4- piperidyl)acetate | (B1) IIIb-Z = CHCH₂C(O)OCH₃ (Combi-Blocks), (B2) hydrolysis, ester coupling | CD₃OD: δ 5.57 (m, 1H), 2.27 (m, 2H), 1.24 (d, 6H), 0.96 (m, 12H) | ++ |
| 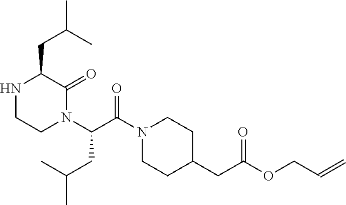 [SMILES: C=CCOC(=O)CC1CCN(C(=O)[C@H](CC(C)C)N2CCN[C@@H](CC(C)C)C2=O)CC1] | 196 | Allyl (1-{(S)-2-[(S)-3 isobutyl-2-oxo-1- piperazinyl]-4- methylvaleryl}-4- piperidyl)acetate | (B1) IIIb-Z = CHCH₂C(O)OCH₃ (Combi-Blocks), (B2) hydrolysis, ester coupling | CD₃OD: δ 6.03-5.88 (m, 1H), 5.57 (m, 1H), 2.35 (m, 2H), 0.96 (m, 12H) | +* |
| 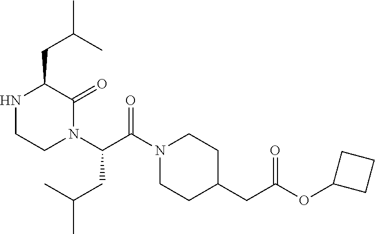 [SMILES: CC(C)C[C@@H]1NCCN([C@@H](CC(C)C)C(=O)N2CCC(CC(=O)OC3CCC3)CC2)C1=O] | 197 | Cyclobutyl (1-{(S)-2-[(S)- 3-isobutyl-2-oxo-1- piperazinyl]-4- methylvaleryl}-4- piperidyl)acetate | (B1) IIIb-Z = CHCH₂C(O)OCH₃ (Combi-Blocks), (B2) hydrolysis, ester coupling | CD₃OD: δ 5.57 (m, 1H), 4.98 (m, 1H), 4.48 (t, 1H), 0.96 (m, 12H) | ++ |
| 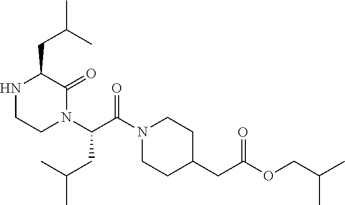 [SMILES: CC(C)COC(=O)CC1CCN(C(=O)[C@H](CC(C)C)N2CCN[C@@H](CC(C)C)C2=O)CC1] | 198 | Isobutyl (1-{(S)-2-[(S)-3- isobutyl-2-oxo-1- piperazinyl]-4- methylvaleryl}-4- piperidyl)acetate | (B1) IIIb-Z = CHCH₂C(O)OCH₃ (Combi-Blocks), (B2) hydrolysis, ester coupling | CD₃OD: δ 5.57 (m, 1H), 3.88 (d, 2H), 2.32 (m, 2H), 0.97 (m, 18H) | ++ |
| 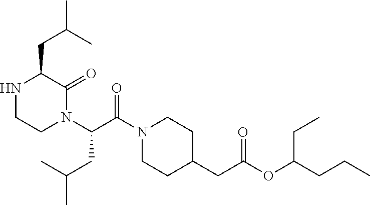 [SMILES: CCCC(CC)OC(=O)CC1CCN(C(=O)[C@H](CC(C)C)N2CCN[C@@H](CC(C)C)C2=O)CC1] | 199 | 1-Ethylbutyl (1-{(S)-2- [(S)-3-isobutyl-2-oxo-1- piperazinyl]-4- methylvaleryl}-4- piperidyl)acetate | (B1) IIIb-Z = CHCH₂C(O)OCH₃ (Combi-Blocks), (B2) hydrolysis, ester coupling | CD₃OD: δ 5.57 (m, 1H), 4.48 (t, 1H), 2.31 (m, 2H, 1.01- 0.87 (m, 18H) | +++ |
| 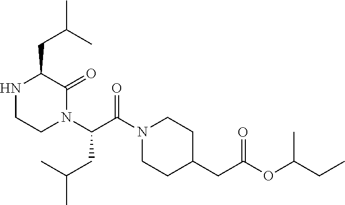 [SMILES: CCC(C)OC(=O)CC1CCN(C(=O)[C@H](CC(C)C)N2CCN[C@@H](CC(C)C)C2=O)CC1] | 200 | sec-Butyl (1-{(S)-2-[(S)- 3-isobutyl-2-oxo-1- piperazinyl]-4- methylvaleryl}-4- piperidyl)acetate | (B1) IIIb-Z = CHCH₂C(O)OCH₃ (Combi-Blocks), (B2) hydrolysis, ester coupling | CD₃OD: δ 5.58 (m, 1H), 2.29 (m, 2H), 1.22 (d, 3H), 1.00- 0.89 (m, 15H) | +++ |
| 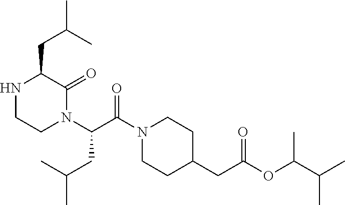 [SMILES: CC(C)C[C@@H]1NCCN([C@@H](CC(C)C)C(=O)N2CCC(CC(=O)OC(C)C(C)C)CC2)C1=O] | 201 | 1,2-Dimethylpropyl (1- {(S)-2-[(S)-3-isobutyl-2- oxo-1-piperazinyl]-4- methylvaleryl}-4- piperidyl)acetate | (B1) IIIb-Z = CHCH₂C(O)OCH₃ (Combi-Blocks), (B2) hydrolysis, ester coupling | CD₃OD: δ 5.58 (m, 1H), 4.74 (m, 1H), 2.30 (m, 2H), 0.96 (m, 18H) | +++ |
| 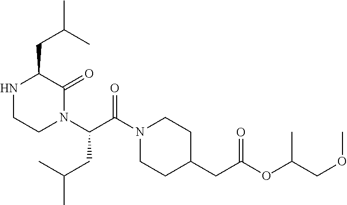 [SMILES: COCC(C)OC(=O)CC1CCN(C(=O)[C@H](CC(C)C)N2CCN[C@@H](CC(C)C)C2=O)CC1] | 202 | 2-Methoxy-1- methylethyl (1-{(S)-2- [(S)-3-isobutyl-2-oxo-1- piperazinyl]-4- methylvaleryl}-4- piperidyl)acetate | (B1) IIIb-Z = CHCH₂C(O)OCH₃ (Combi-Blocks), (B2) hydrolysis, ester coupling | CD₃OD: δ 5.58 (m, 1H), 5.09 (m, 1H), 3.36 (s, 3H), 0.97 (m, 12H) | +* |
| 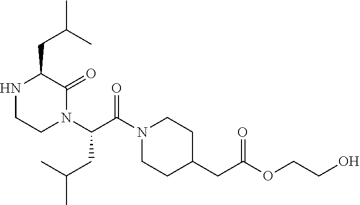 [SMILES: CC(C)C[C@@H]1NCCN([C@@H](CC(C)C)C(=O)N2CCC(CC(=O)OCCO)CC2)C1=O] | 203 | 2-Hydroxyethyl (1-{(S)-2-[(S)-3- isobutyl-2-oxo- 1-piperazinyl]-4- methylvaleryl}-4- piperidyl)acetate | (B1) IIIb-Z = CHCH₂C(O)OCH₃ (Combi-Blocks), (B2) hydrolysis, ester coupling | CD₃OD: δ 5.58 (m, 1H), 4.35 (t, 2H), 2.37 (m, 2H), 0.97 (m, 12H) | ++ |
| 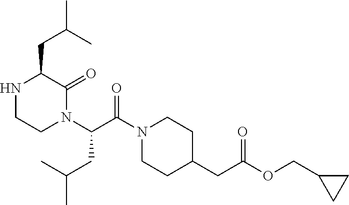 [SMILES: CC(C)C[C@@H]1NCCN([C@@H](CC(C)C)C(=O)N2CCC(CC(=O)OCC3CC3)CC2)C1=O] | 204 | Cydopropylmethyl (1- {(S)-2-[(S)-3-isobutyl-2- oxo-1-piperazinyl]-4- methylvaleryl}-4- piperidyl)acetate | (B1) IIIb-Z = CHCH₂C(O)OCH₃ (Combi-Blocks), (B2) hydrolysis, ester coupling | CD₃OD: δ 5.58 (m, 1H), 3.93 (d, 2H), 0.97 (m, 12H), 0.29 (m, 2H) | ++ |
| 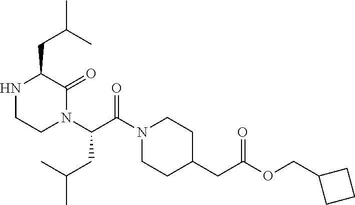 [SMILES: CC(C)C[C@@H]1NCCN([C@@H](CC(C)C)C(=O)N2CCC(CC(=O)OCC3CCC3)CC2)C1=O] | 205 | Cyclobutylmethyl (1- {(S)-2-[(S)-3-isobutyl-2- oxo-1-piperazinyl]-4- methylvaleryl}-4- piperidyl)acetate | (B1) IIIb-Z = CHCH₂C(O)OCH₃ (Combi-Blocks), (B2) hydrolysis, ester coupling | CD₃OD: δ 5.58 (m, 1H), 4.07 (d, 2H), 2.31 (m, 2H), 0.97 (m, 12H) | ++ |
| 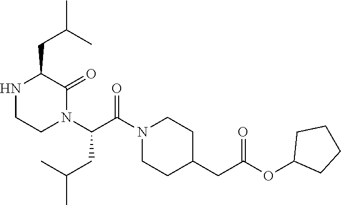 [SMILES: CC(C)C[C@@H]1NCCN([C@@H](CC(C)C)C(=O)N2CCC(CC(=O)OC3CCCC3)CC2)C1=O] | 206 | Cyclopentyl (1-{(S)-2- [(S)-3-isobutyl-2-oxo-1- piperazinyl]-4- methylvaleryl}-4- piperidyl)acetate | (B1) IIIb-Z = CHCH₂C(O)OCH₃ (Combi-Blocks), (B2) hydrolysis, ester coupling | CD₃OD: δ 5.58 (m, 1H), 5.16 (m, 1H), 2.27 (m, 2H), 0.97 (m, 12H) | ++ |
| 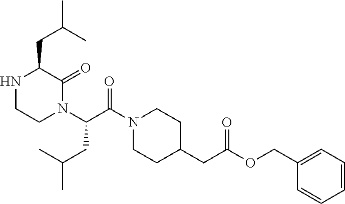 [SMILES: CC(C)C[C@@H]1NCCN([C@@H](CC(C)C)C(=O)N2CCC(CC(=O)OCc3ccccc3)CC2)C1=O] | 207 | Benzyl (1-{(S)-2-[(S)-3- isobutyl-2-oxo-1- piperazinyl]-4- methylvaleryl}-4- piperidyl)acetate | (B1) IIIb-Z = CHCH₂C(O)OCH₃ (Combi-Blocks), (B2) hydrolysis, ester coupling | CD₃OD: δ 5.57 (m, 1H), 5.13 (s, 2H), 2.36 (m, 2H), 0.96 (m, 12H) | ++ |
| 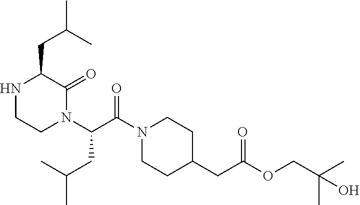 [SMILES: CC(C)C[C@@H]1NCCN([C@@H](CC(C)C)C(=O)N2CCC(CC(=O)OCC(C)(C)O)CC2)C1=O] | 208 | 2-Hydroxy-2- methylpropyl (1-{(S)-2- [(S)-3-isobutyl-2-oxo-1- piperazinyl]-4- methylvaleryl}-4- piperidyl)acetate | (B1) IIIb-Z = CHCH₂C(O)OCH₃ (Combi-Blocks), (B2) hydrolysis, ester coupling | CD₃OD: δ 5.58 (m, 1H), 3.94 (s, 2H), 2.37 (m, 2H), 0.96 (m, 12H) | +* |
| 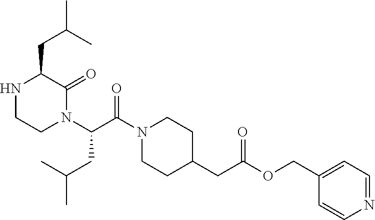 [SMILES: CC(C)C[C@@H]1NCCN([C@@H](CC(C)C)C(=O)N2CCC(CC(=O)OCc3ccncc3)CC2)C1=O] | 209 | (4-Pyridyl)methyl (1- {(S)-2-[(S)-3-isobutyl-2- oxo-1-piperazinyl]-4- methylvaleryl}-4- piperidyl)acetate | (B1) IIIb-Z = CHCH₂C(O)OCH₃ (Combi-Blocks), (B2) hydrolysis, ester coupling | CD₃OD: δ 8.55-8.47 (m, 2H), 7.43 (m, 2H), 5.56 (m, 1H), 0.96 (m, 12H) | ++ |
| 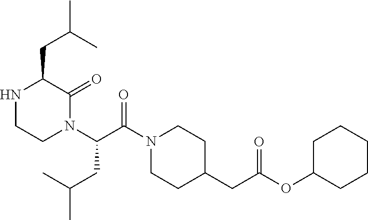 [SMILES: CC(C)C[C@@H]1NCCN([C@@H](CC(C)C)C(=O)N2CCC(CC(=O)OC3CCCCC3)CC2)C1=O] | 210 | Cyclohexyl (1-{(S)-2- [(S)-3-isobutyl-2-oxo-1- piperazinyl]-4- methylvaleryl}-4- piperidyl)acetate | (B1) IIIb-Z = CHCH₂C(O)OCH₃ (Combi-Blocks), (B2) hydrolysis, ester coupling | CD₃OD: δ 5.58 (m, 1H), 4.77 (m, 1H), 2.29 (m, 2H), 0.97 (m, 12H) | +++ |
| 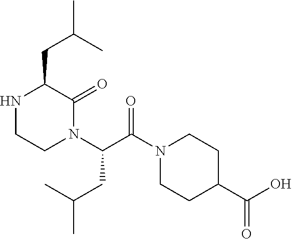 [SMILES: CC(C)C[C@@H]1NCCN([C@@H](CC(C)C)C(=O)N2CCC(C(=O)O)CC2)C1=O] | 211 | 1-{(S)-2-[(S)-3-Isobutyl- 2-oxo-1-piperazinyl]-4- methylvaleryl}-4- piperidinecarboxylic acid | (B1) IIIb-Z = CHC(O)OCH₃, (B2) hydrolysis | CD₃OD: δ 5.53 (m, 1H), 4.46- 4.22 (br m, 1H), 2.31 (m, 1H), 0.89 (m, 12H) | +* |
| 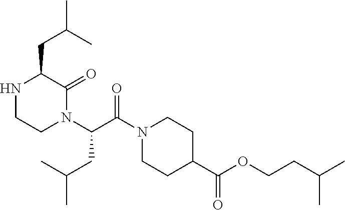 [SMILES: CC(C)CCOC(=O)C1CCN(C(=O)[C@H](CC(C)C)N2CCN[C@@H](CC(C)C)C2=O)CC1] | 212 | Isopentyl 1-{(S)-2-[(S)-3- isobutyl-2-oxo-1- piperazinyl]-4- methylvaleryl}-4- piperidinecarboxylate | (B1) IIIb-Z = CHC(O)OCH₃, (B2) hydrolysis ester coupling | CD₃OD: δ 5.59 (m, 1H), 4.16 (m, 2H), 2.68 (m, 1H), 0.97 (m, 18H) | ++ |
| 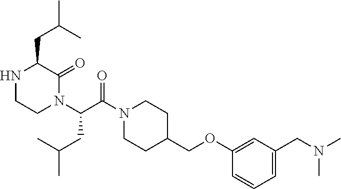 [SMILES: CC(C)C[C@@H]1NCCN([C@@H](CC(C)C)C(=O)N2CCC(COc3cccc(CN(C)C)c3)CC2)C1=O] | 213 | (S)-1-[(S)-1-{[4-({m- [(Dimethyl- amino)meth- yl]phenoxy}methyl)-1- piperidyl]carbonyl}-3- methylbutyl]-3- isobutyl-2-piperazinone | Z = CHCH₂OH, Mitsunobu reaction with m- hydroxy- benzaldehyde, reductive amination with dimethylamine | CD₃OD: δ 7.27 (t, 1H), 7.00- 6.86 (br m, 3H), 5.58 (m, 1H), 3.88 (d, 2H), 3.68 (s, 2H), 2.41 (s, 6H), | +++ |
| | | | | 0.95 (m, | |
| | | | | 12H) | |
| 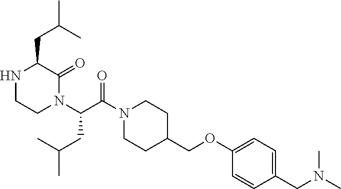 [SMILES: CC(C)C[C@@H]1NCCN([C@@H](CC(C)C)C(=O)N2CCC(COc3ccc(CN(C)C)cc3)CC2)C1=O] | 214 | (S)-1-[(S)-1-{ [4-({p- [(Dimethyl- amino)meth- yl]phenoxy} methyl)-1- piperidyl] carbonyl}-3- methylbutyl]-3- isobutyl-2- piperazinone | Z = CHCH₂OH, Mitsunobu reaction with p- hydroxy- benzaldehyde, reductive amination with dimethylamine | CD₃OD: δ 7.21 (d, 2H), 6.88 (d, 2H), 5.59 (m, 1H), 3.85 (d, 2H), 3.40 (s, 2H), 2.21 (s, 6H), 0.95 (m, | +++ |
| | | | | 12H) | |
| 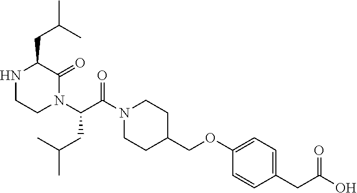 [SMILES: CC(C)C[C@@H]1NCCN([C@@H](CC(C)C)C(=O)N2CCC(COc3ccc(CC(=O)O)cc3)CC2)C1=O] | 215 | {p-[(1-{(S)-2- [(S)-3- Isobutyl-2- oxo-1- piperazinyl]-4- methylvaleryl}- 4- piperidyl) methoxy] phenyl} acetic acid | Z = CHCH₂OH, Mitsunobu reaction with methyl 4- hydroxyphenyl- acetate, ester hydrolysis | CD₃OD: δ 7.20 (d, 2H), 6.81 (d, 2H), 5.58 (m, 1H), 3.83 (d, 2H), 3.39 (s, 2H), 0.95 (m, 12H) | +* |
| 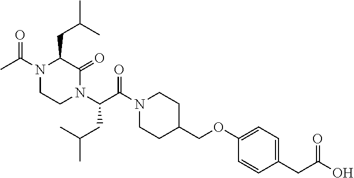 [SMILES: CC(=O)N1CCN([C@@H](CC(C)C)C(=O)N2CCC(COc3ccc(CC(=O)O)cc3)CC2)C(=O)[C@@H]1CC(C)C] | 216 | {p-[(1-{(S)-2- [(S)-4- Acetyl-3- isobutyl-2-oxo- 1-piperazinyl]- 4-methyl- valeryl}-4- piperidyl) methoxy] phenyl} acetic acid | Z = CHCH₂OH, Mitsunobu reaction with methyl 4- hydroxyphenyl- acetate, ester hydrolysis | CD₃OD: δ 7.20 (d, 2H), 6.81 (d, 2H), 5.58 (m, 1H), 5.04 (m, 1H), 3.39 (s, 2H), 2.13 (s, 3H), 0.97 (m, | +* |
| | | | | 12H) | |
| 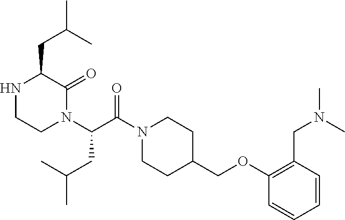 [SMILES: CC(C)C[C@@H]1NCCN([C@@H](CC(C)C)C(=O)N2CCC(COc3ccccc3CN(C)C)CC2)C1=O] | 217 | (S)-1-[(S)-1-{[4-({o- [(Dimethyl- amino)meth- yl]phenoxy}methyl)-1- piperidyl]carbonyl}-3- methylbutyl]-3- isobutyl-2-piperazinone | Z = CHCH₂OH, tosylation with tosyl choride, substitution reaction with salicylaldehyde, reductive amination with dimethylamine | CD₃OD: δ 7.42-7.30 (br m, 2H), 7.06 (d, 1H), 6.99 (t, 1H), 5.58 (m, 1H), 3.95 (m, 4H), 2.60 (s, 6H), 0.95 (m, 12H) | +++ |
| 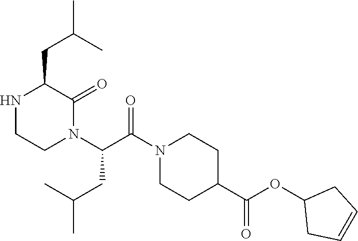 [SMILES: CC(C)C[C@@H]1NCCN([C@@H](CC(C)C)C(=O)N2CCC(C(=O)OC3CC=CC3)CC2)C1=O] | 218 | 3-Cyclopenten-1-yl 1-{(S)-2-[(S)- 3-isobutyl-2- oxo-1-piperazinyl]-4- methylvaleryl}-4- piperidinecarboxylate | (B1) IIIb-Z = CHC(O)OCH₃, (B2) hydrolysis, ester coupling | CD₃OD: δ 5.74 (s, 2H), 5.58 (m, 1H), 2.64 (m, 1H), 0.98 (m, 12H) | ++ |
| 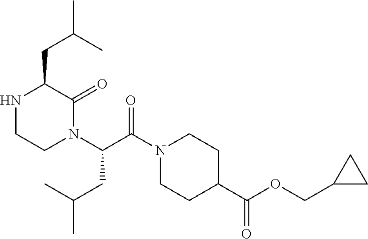 [SMILES: CC(C)C[C@@H]1NCCN([C@@H](CC(C)C)C(=O)N2CCC(C(=O)OCC3CC3)CC2)C1=O] | 219 | Cydopropylmethyl 1- {(S)-2-[(S)-3-isobutyl-2- oxo-1-piperazinyl]-4- methylvaleryl}-4- piperidinecarboxylate | (B1) IIIb-Z = CHC(O)OCH₃, (B2) hydrolysis, ester coupling | CD₃OD: δ 5.60 (m, 1H), 2.70 (m, 1H), 0.98 (m, 12H), 0.31 (m, 2H) | +* |
| 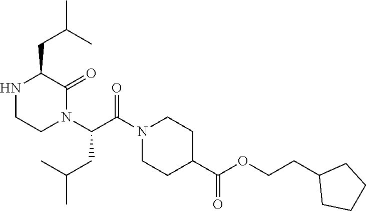 [SMILES: CC(C)C[C@@H]1NCCN([C@@H](CC(C)C)C(=O)N2CCC(C(=O)OCCC3CCCC3)CC2)C1=O] | 220 | 2-Cyclopentylethyl 1- {(S)-2-[(S)-3-isobutyl-2- oxo-1-piperazinyl]-4- methylvaleryl}-4- piperidinecarboxylate | (B1) IIIb-Z = CHC(O)OCH₃, (B2) hydrolysis, ester coupling | CD₃OD: δ 5.58 (m, 1H), 4.13 (m, 2H), 2.67 (m, 1H), 0.97 (m, 12H) | +* |
| 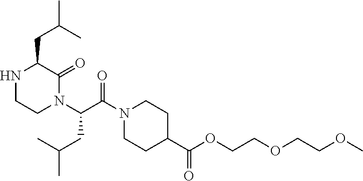 [SMILES: COCCOCCOC(=O)C1CCN(C(=O)[C@H](CC(C)C)N2CCN[C@@H](CC(C)C)C2=O)CC1] | 221 | 2-(2- Methoxyethoxy)ethyl 1- {(S)-2-[(S)-3-isobutyl-2- oxo-1-piperazinyl]-4- methylvaleryl}-4- piperidinecarboxylate | (B1) IIIb-Z = CHC(O)OCH₃, (B2) hydrolysis, ester coupling | CD₃OD: δ 5.58 (m, 1H), 3.37 (s, 3H), 2.71 (m, 1H), 0.97 (m, 12H) | ND |
| 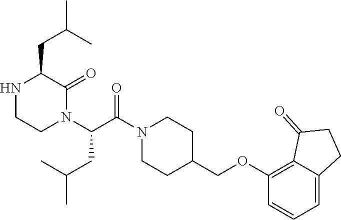 [SMILES: CC(C)C[C@@H]1NCCN([C@@H](CC(C)C)C(=O)N2CCC(COc3cccc4c3C(=O)CC4)CC2)C1=O] | 222 | (S)-1-[(S)-3-Methyl-1- ({4-[(3-oxo-4- indanyloxy)methyl]-1- piperidyl}car- bonyl)butyl]-3- isobutyl-2- piperazinone | (B1) IIIb-Z = CHCH₂OH, (B2) Mitsunobu reaction | CD₃OD: 5.60 (m, 1H), 3.87 (d, 2H), 2.63 (d, 2H), 0.97 (m, 12H) | ++ |
| 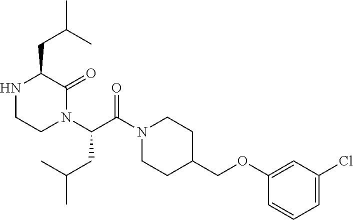 [SMILES: CC(C)C[C@@H]1NCCN([C@@H](CC(C)C)C(=O)N2CCC(COc3cccc(Cl)c3)CC2)C1=O] | 223 | (S)-1-[(S)-1-({4-[(m- Chlorophenoxy)meth- yl]-1-piperidyl}car- bonyl)- 3-methylbutyl]-3- isobutyl-2-piperazinone | (B1) IIIb-Z = CHCH₂OH, (B2) Mitsunobu reaction | CD₃OD: δ 6.95 (s, 1H), 5.60 (m, 1H), 3.87 (d, 2H), 0.97 (m, 12H) | +++ |
| 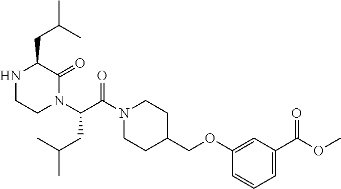 [SMILES: COC(=O)c1cccc(OCC2CCN(C(=O)[C@H](CC(C)C)N3CCN[C@@H](CC(C)C)C3=O)CC2)c1] | 224 | Methyl m-[(1-{(S)-2- [(S)-3-isobutyl-2-oxo-1- piperazinyl]-4- methylvaleryl}-4- piperidyl)meth- oxy]benzoate | (B1) IIIb-Z = CHCH₂OH, (B2) Mitsunobu reaction | CD₃OD: δ 7.54 (s, 1H), 5.60 (m, 1H), 3.92 (m, 5H), 0.97 (m, 12H) | +++ |
| 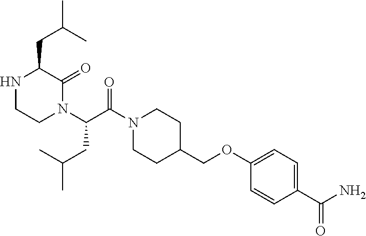 [SMILES: CC(C)C[C@@H]1NCCN([C@@H](CC(C)C)C(=O)N2CCC(COc3ccc(C(N)=O)cc3)CC2)C1=O] | 225 | p-[(1-{(S)-2-[(S)-3- Isobutyl-2-oxo-1- piperazinyl]-4- methylvaleryl}-4- piperidyl)meth- oxy]benzamide | (B1) IIIb-Z = CHCH₂OH, (B2) Mitsunobu reaction | CD₃OD: δ 7.85 (d, 2H), 5.60 (m, 1H), 3.94 (d, 2H), 0.97 (m, 12H) | ++ |
| 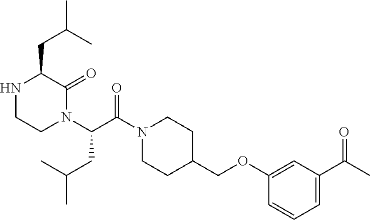 [SMILES: CC(=O)c1cccc(OCC2CCN(C(=O)[C@H](CC(C)C)N3CCN[C@@H](CC(C)C)C3=O)CC2)c1] | 226 | (S)-1-[(S)-1-({4-[(m- Acetylphenoxy)methyl]- 1-piperidyl}carbonyl)-3- methylbutyl]-3- isobutyl-2-piperazinone | (B1) IIIb-Z = CHCH₂OH, (B2) Mitsunobu reaction | CD₃OD: δ 5.60 (m, 1H), 3.94 (d, 2H), 2.60 (s, 3H), 0.97 (m, 12H) | +++ |
| 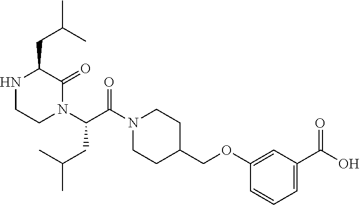 [SMILES: CC(C)C[C@@H]1NCCN([C@@H](CC(C)C)C(=O)N2CCC(COc3cccc(C(=O)O)c3)CC2)C1=O] | 227 | m-[(1-{(S)-2-[(S)-3- Isobutyl-2-oxo-1- piperazinyl]-4- methylvaleryl}-4- piperidyl)meth- oxy]benzoic acid | (B1) IIIb-Z = CHCH₂OH, (B2) Mitsunobu reaction | CD₃OD: δ 7.53 (m, 2H), 5.60 (m, 1H), 3.91 (d, 2H), 0.96 (m, 12H) | +* |
| 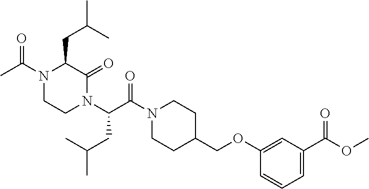 [SMILES: COC(=O)c1cccc(OCC2CCN(C(=O)[C@H](CC(C)C)N3CCN(C(C)=O)[C@@H](CC(C)C)C3=O)CC2)c1] | 228 | Methyl m-[(1-{(S)-2- [(S)-4-acetyl-3-isobutyl- 2-oxo-1-piperazinyl]-4- methylvaleryl}-4- piperidyl)meth- oxy]benzoate | (B1) IIIb-Z = CHCH₂OH, (B2) Mitsunobu reaction | CDCl₃: δ 5.71-5.51 (m, 1H), 3.92 (s, 3H), 2.11 (m, 3H), 0.98 (m, 12H) | +++ |
| 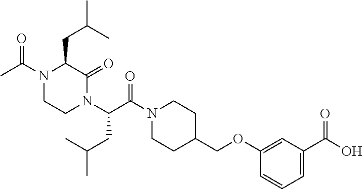 [SMILES: CC(=O)N1CCN([C@@H](CC(C)C)C(=O)N2CCC(COc3cccc(C(=O)O)c3)CC2)C(=O)[C@@H]1CC(C)C] | 229 | m-[(1-{(S)-2-[(S)-4- Acetyl-3-isobutyl-2-oxo- 1-piperazinyl]-4- methylvaleryl}-4- piperidyl)meth- oxy]benzoic acid | (B1) IIIb-Z = CHCH₂OH, (B2) Mitsunobu reaction | CD₃OD: δ 7.53 (m, 2H), 5.59 (m, 1H), 2.14 (m, 3H), 0.98 (m, 12H) | ND |
| 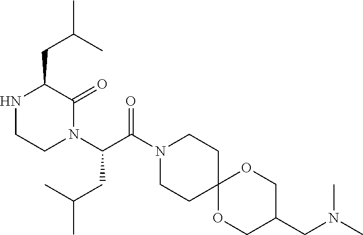 [SMILES: CC(C)C[C@@H]1NCCN([C@@H](CC(C)C)C(=O)N2CCC3(CC2)OCC(CN(C)C)CO3)C1=O] | 230 | (S)-1-[(S)-1-({3- [(Dimethyl- amino)methyl]-1,5- dioxa-9-aza-9- spiro[5.5]un- decyl}carbonyl)-3- methylbutyl]-3- isobutyl-2-piperazinone | (B1) IIIb-Z = C═O, (B2) ketalization using (2,2- dimethyl-1,3- dioxan-5- yl)methanol (Combi-Blocks), tosylation of alcohol, substitution of tosylate using HNMe₂ | CD₃OD: δ 5.57 (m, 1H), 3.98 (d, 2H), 2.88 (m, 1H), 2.36 (d, 2H), 2.26 (s, 6H), 0.95 (m, 12H) | ++++ |
| 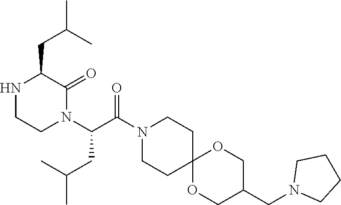 [SMILES: CC(C)C[C@@H]1NCCN([C@@H](CC(C)C)C(=O)N2CCC3(CC2)OCC(CN2CCCC2)CO3)C1=O] | 231 | (S)-1-[(S)-3-methyl-1- ({3-[(1- pyrrolidinyl)methyl]- l,5-dioxa-9-aza-9- spiro[5.5]un- decyl}carbonyl)butyl]-3- isobutyl-2- piperazinone | (B1) IIIb-Z = C═O, (B2) ketalization using (2,2- dimethyl-1,3- dioxan-5- yl)methanol (Combi-Blocks), tosylation of alcohol, substitution of tosylate using pyrrolidine | CD₃OD: δ 5.57 (m, 1H), 3.42 (d, 2H), 1.65 (m, 4H), 1.00 (m, 12H) | ++++ |
| 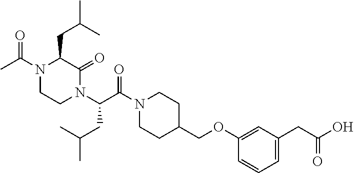 [SMILES: CC(=O)N1CCN([C@@H](CC(C)C)C(=O)N2CCC(COc3cccc(CC(=O)O)c3)CC2)C(=O)[C@@H]1CC(C)C] | 303 | {m-[(1-{(S)-2-[(S)-4- Acetyl-3-isobutyl-2-oxo- 1-piperazinyl]-4- methylvaleryl}-4- piperidyl)meth- oxy]phenyl}acetic acid | (B1) IIIb-Z = CHCH₂OH (B2) Mitsunobu reaction with Methyl (m- hydroxy- phenyl)acetate, ester hydrolysis | CD₃OD: δ 7.14 (t, 1H), 5.58 (m, 1H), 2.13 (m, 3H), 0.98 (m, 12H) | +* |
| 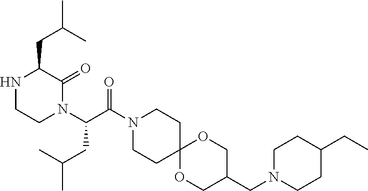 [SMILES: CCC1CCN(CC2COC3(CCN(C(=O)[C@H](CC(C)C)N4CCN[C@@H](CC(C)C)C4=O)CC3)OC2)CC1] | 304 | (S)-1-[(S)-1-({3-[(4- Ethyl-1- piperidyl)methyl]-1,5- dioxa-9-aza-9- spiro[5.5]un- decyl}carbonyl)-3- methylbutyl]-3- isobutyl-2-piperazinone | (B1) IIIb-Z = C═O (Combi-Blocks) (B2) ketalization with (2,2-Dimethyl- 1,3-dioxan-5- yl)methanol (Combi-Blocks), mesylation of alcohol, substitution of mesylate using 4- Ethylpiperidine (AstaTech) | CD₃OD: δ 5.59 (dd, 1H), 3.98 (br d, 2H), 2.33 (d, 2H), 1.01- 0.89 (m, 15H) | ++++ |
| 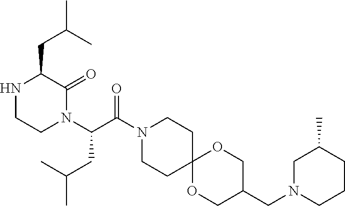 [SMILES: CC(C)C[C@@H]1NCCN([C@@H](CC(C)C)C(=O)N2CCC3(CC2)OCC(CN2CCC[C@@H](C)C2)CO3)C1=O] | 305 | (S)-1-[(S)-1-[(3-{[(R)-3- Methyl-1- piperidyl]methyl}-1,5- dioxa-9-aza-9- spiro[5.5]un- decyl)carbonyl]-3- methylbutyl]-3- isobutyl-2-piperazinone | (B1) IIIb-Z = C═O (Combi-Blocks) (B2) ketalization with (2,2-Dimethyl- 1,3-dioxan-5- yl)methanol (Combi-Blocks), mesylation of alcohol, substitution of mesylate using (R)- 3-Methylpiperidine (AstaTech) | CD₃OD: δ 5.59 (m, 1H), 4.03- 3.93 (m, 2H), 2.95- 2.80 9m, 3H), 2.40-2.33 (m, 2H) | ++++ |
| 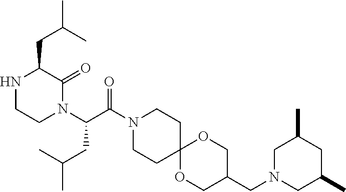 [SMILES: CC(C)C[C@@H]1NCCN([C@@H](CC(C)C)C(=O)N2CCC3(CC2)OCC(CN2C[C@H](C)C[C@H](C)C2)CO3)C1=O] | 306 | (S)-1-[(S)-1-({3-[(3,5- Dimethyl-1- piperidyl)methyl]-1,5- dioxa-9-aza-9- spiro[5.5]un- decyl}carbonyl)-3- methylbutyl]-3- isobutyl-2-piperazinone | (B1) IIIb-Z = C═O (Combi-Blocks) (B2) ketalization with (2,2-Dimethyl- 1,3-dioxan-5- yl)methanol (Combi-Blocks), mesylation of alcohol, substitution of mesylate using 3,5- cis- Dimethylpiperidine (AstaTech) | CD₃OD: δ 5.58 (dd, 1H), 2.34 (d, 2H), 0.97 (m, 12H), 0.89 (d, 6H) | ++++ |
| 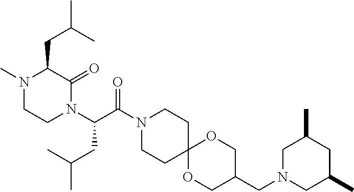 | 307 | (S)-1-[(S)-1-({3-[(3,5- Dimethyl-1- piperidyl)methyl]-1,5- dioxa-9-aza-9- spiro[5.5]un- decyl}carbonyl)-3- methylbutyl]-3- isobutyl-4-methyl-2- piperazinone | (B1) IIIb-Z = C═O (Combi-Blocks) (B2) ketalization with (2,2-Dimethyl- 1,3-dioxan-5- yl)methanol (Combi-Blocks), mesylation of alcohol, substitution of mesylate using cis- 3,5- Dimethylpiperidine | CDCl₃: δ 5.57 (m, 1H), 2.49 (m, 1H), 2.33 (s, 3H), 0.97- 0.79 (m, 18H) | ++++ |
| | | | (AstaTech) | | |
| 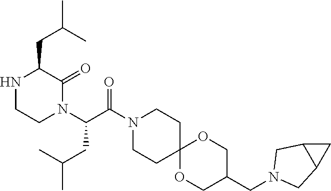 [SMILES: CC(C)C[C@@H]1NCCN([C@@H](CC(C)C)C(=O)N2CCC3(CC2)OCC(CN2CC4CC4C2)CO3)C1=O] | 308 | (S)-1-[(S)-1-[(3-{(3- Azabicyclo[3.1.0]hex-3- yl)methyl}-1,5-dioxa-9- aza-9- spiro[5.5]un- decyl)carbonyl]-3- methylbutyl]-3- isobutyl-2-piperazinone | (B1) IIIb-Z = C═O (Combi-Blocks) (B2) ketalization with (2,2-Dimethyl- 1,3-dioxan-5- yl)methanol (Combi-Blocks), mesylation of alcohol, substitution of mesylate using 3- Azabicyclo[3.1.0]hex- ane (AstaTech) | CD₃OD: δ 5.61-5.55 (m, 1H), 2.66 (m, 1H), 0.97 (m, 12H), 0.40-0.31 (m, 1H) | ++++ |
| 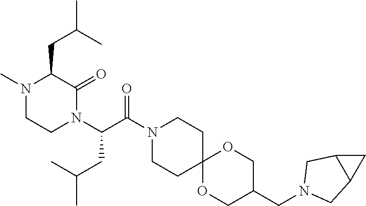 [SMILES: CC(C)C[C@H]1C(=O)N([C@@H](CC(C)C)C(=O)N2CCC3(CC2)OCC(CN2CC4CC4C2)CO3)CCN1C] | 309 | (S)-1-[(S)-1-[(3-{(3- Azabicyclo[3.1.0]hex-3- yl)methyl}-1,5-dioxa-9- aza-9- spiro[5.5]un- decyl)carbonyl]-3- methylbutyl]-3- isobutyl-4-methyl-2- piperazinone | (B1) IIIb-Z = C═O (Combi-Blocks) (B2) ketalization with (2,2-Dimethyl- 1,3-dioxan-5- yl)methanol (Combi-Blocks), mesylation of alcohol, substitution of mesylate using 3- Azabicyclo[3.1.0]hex- ane (AstaTech) | CDCl₃: δ 5.62-5.54 (m, 1H), 2.34 (s, 3H), 0.98- 0.87 (m, 12H), 0.38-0.26 (m, 1H) | ++++ |
| 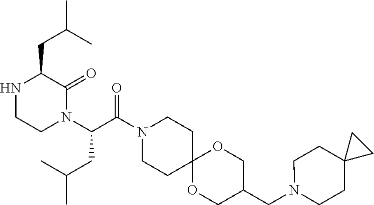 [SMILES: CC(C)C[C@@H]1NCCN([C@@H](CC(C)C)C(=O)N2CCC3(CC2)OCC(CN2CCC4(CC2)CC4)CO3)C1=O] | 310 | (S)-1-[(S)-1- [(3-{(6-Aza-6- spiro[2.5]octyl)methyl}- 1,5-dioxa-9-aza-9- spiro[5.5]un- decyl)carbonyl]-3- methylbutyl]-3- isobutyl-2-piperazinone | (B1) IIIb-Z = C═O (Combi-Blocks) (B2) ketalization with (2,2-Dimethyl- 1,3-dioxan-5- yl)methanol (Combi-Blocks), mesylation of alcohol, substitution of mesylate using 6- Azaspiro[2.5]oc- tane (AstaTech) | CD₃OD: δ 5.62-5.56 (m, 1H), 2.43-2.37 (m, 2H), 0.97 (m, 12H), 0.31 (s, 4H) | ++++ |
| 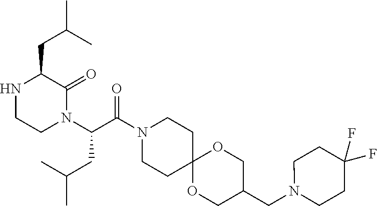 [SMILES: CC(C)C[C@@H]1NCCN([C@@H](CC(C)C)C(=O)N2CCC3(CC2)OCC(CN2CCC(F)(F)CC2)CO3)C1=O] | 311 | (S)-1-[(S)-1-({3-[(4,4- Difluoro-1- piperidyl)methyl]-1,5- dioxa-9-aza-9- spiro[5.5]un- decyl}carbonyl)-3- methylbutyl]-3- isobutyl-2-piperazinone | (B1) IIIb-Z = C═O (Combi-Blocks) (B2) ketalization with (2,2-Dimethyl- 1,3-dioxan-5- yl)methanol (Combi-Blocks), mesylation of alcohol, substitution of mesylate using 4,4- Difluoropiperidine (AstaTech) | CD₃OD: δ 5.62-5.55 (m, 1H), 2.96-2.76 (m, 3H), 2.43 (d, 2H), 0.97 (m, 12H) | *+ |
| 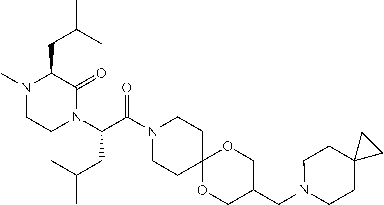 [SMILES: CC(C)C[C@H]1C(=O)N([C@@H](CC(C)C)C(=O)N2CCC3(CC2)OCC(CN2CCC4(CC2)CC4)CO3)CCN1C] | 312 | (S)-1-[(S)-1- [(3-{(6-Aza-6- spiro[2.5]oc- tyl)methyl}- 1,5-dioxa-9-aza-9- spiro[5.5]undecyl)car- bonyl]-3- methylbutyl]-3- isobutyl-4-methyl-2- piperazinone | (B1) IIIb-Z = C═O (Combi-Blocks) (B2) ketalization with (2,2-Dimethyl- 1,3-dioxan-5- yl)methanol (Combi-Blocks), mesylation of alcohol, substitution of mesylate using 6- Azaspiro[2.5]octane (AstaTech) | CDCl₃: δ 5.62-5.55 (m, 1H), 2.34 (s, 3H), 0.98- 0.87 (m, 12H), 0.26 (s, 4H) | ++++ |
| 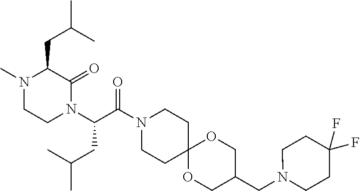 [SMILES: CC(C)C[C@H]1C(=O)N([C@@H](CC(C)C)C(=O)N2CCC3(CC2)OCC(CN2CCC(F)(F)CC2)CO3)CCN1C] | 313 | (S)-1-[(S)-1-({3-[(4,4- Difluoro-1- piperidyl)methyl]-1,5- dioxa-9-aza-9- spiro[5.5]undecyl}car- bonyl)-3- methylbutyl]-3- isobutyl-4-methyl-2- piperazinone | (B1) IIIb-Z = C═O (Combi-Blocks) (B2) ketalization with (2,2-Dimethyl- 1,3-dioxan-5- yl)methanol (Combi-Blocks), mesylation of alcohol, substitution of mesylate using 4,4- Difluoropiperidine (AstaTech) | CDCl₃: δ 5.62-5.55 (m, 1H), 2.86-2.80 (m, 1H), 2.35 (s, 3H), 0.98- 0.87 (m, 12H) | ND |
| 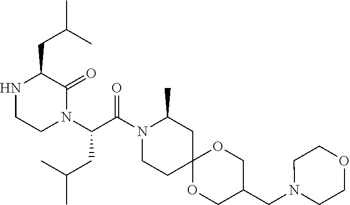 [SMILES: CC(C)C[C@@H]1NCCN([C@@H](CC(C)C)C(=O)N2CCC3(C[C@@H]2C)OCC(CN2CCOCC2)CO3)C1=O] | 314 | (S)-1-[(S)-1-({(S)-8- Methyl-3- (morpholinomethyl)- 1,5-dioxa-9-aza-9- spiro[5.5]undecyl}car- bonyl)-3- methylbutyl]-3- isobutyl-2-piperazinone | (B1) IIIb-Z = C═O (HCl salt, AstaTech) (B2) ketalization with (2,2-Dimethyl- 1,3-dioxan-5- yl)methanol (Combi-Blocks), mesylation of alcohol, substitution of mesylate using Morpholine | CD₃OD: δ 5.54 (m, 1H), 3.68 (m, 6H), 2.55-1.87 (br m, 9H), 0.95 (m, 12H) | ++++ |
| 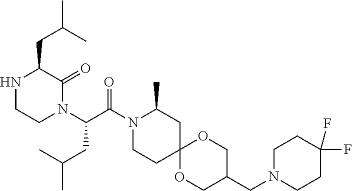 [SMILES: CC(C)C[C@@H]1NCCN([C@@H](CC(C)C)C(=O)N2CCC3(C[C@@H]2C)OCC(CN2CCC(F)(F)CC2)CO3)C1=O] | 315 | (S)-1-[(S)-1- ({(S)-3-[(4,4- Difluoro-1- piperidyl)methyl]-8- methyl-1,5-dioxa-9-aza- 9- spiro[5.5]undecyl}car- bonyl)-3- methylbutyl]-3- isobutyl-2-piperazinone | (B1) IIIb-Z = C═O (HCl salt, AstaTech) (B2) ketalization with (2,2-Dimethyl- 1,3-dioxan-5- yl)methanol (Combi-Blocks), mesylation of alcohol, substitution of mesylate using 4,4- Difluoropiperidine (AstaTech) | CD₃OD: δ 5.54 (m, 1H), 4.11- 3.59 (br m, 5H), 3.05-2.79 (br m, 2H), 0.95 (m, 12H) | ++++ |
| 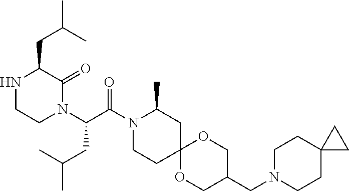 [SMILES: CC(C)C[C@@H]1NCCN([C@@H](CC(C)C)C(=O)N2CCC3(C[C@@H]2C)OCC(CN2CCC4(CC2)CC4)CO3)C1=O] | 316 | (S)-1-[(S)-1-{[(S)-3-{(6- Aza-6- spiro[2.5]octyl)methyl}- 8-methyl-1,5-dioxa-9- aza-9- spiro[5.5]undecyl]car- bonyl}-3- methylbutyl]-3- isobutyl-2-piperazinone | (B1) IIIb-Z = C═O (HCl salt, AstaTech) (B2) ketalization with (2,2-Dimethyl- 1,3-dioxan-5- yl)methanol (Combi-Blocks), mesylation of alcohol, substitution of mesylate using 6- Azaspiro[2.5]oc- tane (AstaTech) | CD₃OD: δ 5.55 (m, 1H), 4.09- 3.83 (br m, 2H), 2.61-1.88 (br m, 9H), 0.95 (m, 12H), 0.29 (s, 4H) | ++++ |
| 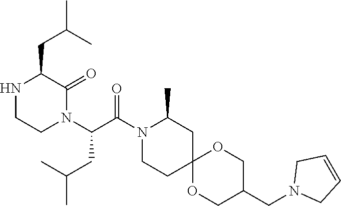 [SMILES: CC(C)C[C@@H]1NCCN([C@@H](CC(C)C)C(=O)N2CCC3(C[C@@H]2C)OCC(CN2CC=CC2)CO3)C1=O] | 317 | (S)-1-[(S)-1- ({(S)-3-[(2,5- Dihydro-1H-pyrrol-1- yl)methyl]-8-methyl- 1,5-dioxa-9-aza-9- spiro[5.5]undecyl}car- bonyl)-3- methylbutyl]-3- isobutyl-2-piperazinone | (B1) IIIb-Z = C═O (HCl salt, AstaTech) (B2) ketalization with (2,2-Dimethyl- 1,3-dioxan-5- yl)methanol (Combi-Blocks), mesylation of alcohol, substitution of mesylate using 2,5- Dihydro-1H- pyrrole (AstaTech) | CD₃OD: δ 5.81 (s, 2H), 5.52 (m, 1H), 4.12-3.89 (br m, 2H), 0.95 (m, 12H) | ++++ |
| 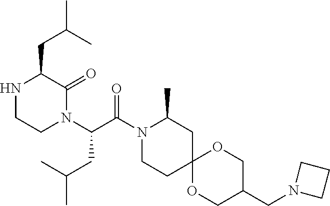 [SMILES: CC(C)C[C@@H]1NCCN([C@@H](CC(C)C)C(=O)N2CCC3(C[C@@H]2C)OCC(CN2CCC2)CO3)C1=O] | 318 | (S)-1-[(S)-1-({(S)-3-[(1- Azetidinyl)methyl]-8- methyl-1,5-dioxa-9-aza- 9- spiro[5.5]undecyl}car- bonyl)-3- methylbutyl]-3- isobutyl-2-piperazinone | (B1) IIIb-Z = C═O (HCl salt, AstaTech) (B2) ketalization with (2,2-Dimethyl- 1,3-dioxan-5- yl)methanol (Combi-Blocks), mesylation of alcohol, substitution of mesylate using Azetidine | CD₃OD: δ 5.52 (m, 1H), 2.59- 1.97 (br m, 6H), 1.91-1.42 (br m, 9H), 0.95 (m, 12H) | ++++ |
| 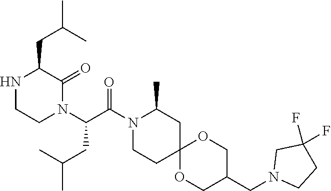 [SMILES: CC(C)C[C@@H]1NCCN([C@@H](CC(C)C)C(=O)N2CCC3(C[C@@H]2C)OCC(CN2CCC(F)(F)C2)CO3)C1=O] | 319 | (S)-1-[(S)-1-({(S)-3- [(3,3-Difluoro-1- pyrrolidinyl)methyl]-8- methyl-1,5-dioxa-9-aza- 9- spiro[5.5]undecyl}car- bonyl)- 3-methylbutyl]-3- isobutyl-2-piperazinone | (B1) IIIb-Z = C═O (HCl salt, AstaTech) (B2) ketalization with (2,2-Dimethyl- 1,3-dioxan-5- yl)methanol (Combi-Blocks), mesylation of alcohol, substitution of mesylate using 3,3- Difluoropyrrolidine (AstaTech) | CD₃OD: δ 5.53 (m, 1H), 4.10- 3.88 (br m, 2H), 3.87-3.60 (br m, 5H), 0.95 (m, 12H) | ++++ |

All starting materials for precursor II and III have been obtained from Sigma-Aldrich unless otherwise noted in Table 2 and 3 above.

The biological properties of compounds were investigated by way of the experimental protocols described below:

### Colony Formation Assay (CFA)

Cell culture: CaSki cells were obtained from CLS GmbH, Eppelheim, Germany (cat. 300145) and subcultured in ready-to-use RPMI1640 Media (CLS GmbH cat. 820700) after addition of “Antibiotic Antimycotic Solution” (Sigma-Aldrich, St Louis, USA, cat. A5955) at 1:100 dilution. CaSki cells were expanded and aliquots kept frozen in liquid nitrogen according to manufacturer's instructions. Once thawed, aliquots were passaged every second or third day at a seed density of 20′000 cells/cm²; and used for a maximum of twenty passages.

Preparation of the cells for CFA: Cell culture flasks were rinsed twice with Ca++/Mg++-free Phosphate-Buffered Saline (DPBS, CLS GmbH cat. 860015) and incubated with Accutase (CLS GmbH cat. 830100) for 15 min at 37° C. Cells were resuspended in a four-fold volume of ready-to use RPMI, centrifuged at 300 g for 10 minutes, the supernatant discarded and the cells resuspended in ready-to-use RPMI1640 by pipetting up and down ten times with a serological pipette. Cell density was determined with a Via-1 Cassette (Chemometec, Allerod, Denmark) on a Nucleocounter NC 3000 (Chemometec). The required amount of cells was first diluted 1:10 in ready-to-use RPMI; cells pipetted five times up-down with a serological pipette; and the 1:10 solution added to the final volume needed for the whole assay setup. Cells were mixed again by pipetting up and down twenty times with a serological pipette. The final cell density was 70 cells/ml.

Seeding: Cells were seeded column-wise in 6-well plates, while triplicates were treated row-wise. Three ml cell suspension solution (210 cells) were added per well.

Treatment: Compound stock solutions were prepared at a concentration of 30 mM in 50% DMSO/50% water; and diluted in the same solution so that the volume added to the wells was of 5 μl and the final DMSO well concentration 0.08%. Untreated cells (negative controls) were incubated in a) medium only; and b) 0.08% DMSO. Test performance was monitored by a standard treatment with a fixed concentration of a reference compound (10 μM Compound 57) that resulted in ˜80-90% inhibition of colony formation. Tests showing less than 75% or more than 95% inhibition were repeated. The plates were swirled gently after addition of the compounds and the cells incubated for 14 days at 37° C. Culture incubation solutions were replaced after one week.

Staining: Colonies were washed twice with ice-cold DPBS and fixed on ice with 1 ml ice-cold 10% methanol solution for 30 min. The methanol solution was removed and colonies incubated with 0.1% Crystal Violet/DBPS for 20 min at room temperature. The wells were rinsed with water at room temperature, let dry and colonies were counted. Compounds e.g., compounds of Table 2 and 3 were classified as follows:

| | |
|---|---|
| ++++ | more than 90% inhibition of colony formation at a concentration |
| | of 6 μM |
| +++ | more than 90% inhibition of colony formation at a concentration |
| | of 50 μM |
| ++ | more than 50% inhibition of colony formation at a concentration |
| | of 50 μM |
| +* | greater than 0% but less than or equal to 50% inhibition of |
| | colony formation at a concentration of 50 μM |
| *+ | greater than 0% but less than or equal to 50% inhibition of |
| | colony formation at a concentration of 0.7 μM |
| ND | activity not detectable at 0.7 μM in this assay |

### Tumor Growth Inhibition in a Patient-Derived Xenograft Model of Head and Neck Cancer

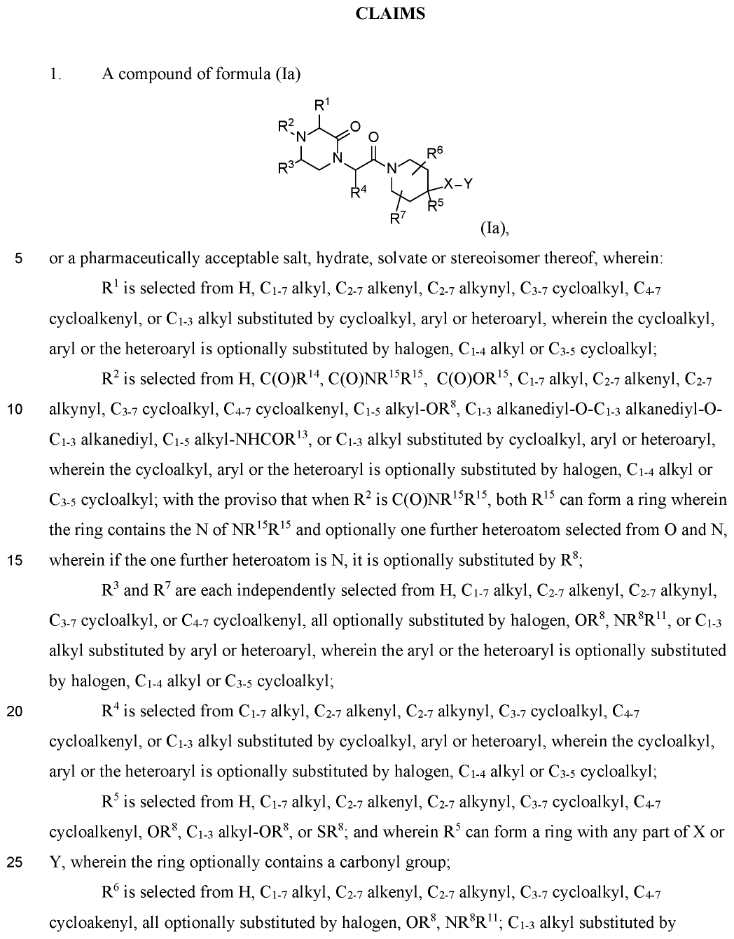

NMRI nude mice bearing HN11873 subcutaneous tumors (Experimental Pharmacology and Oncology Berlin Buch GmbH, Berlin, Germany) were treated p.o. BID with either vehicle (control) or 30 mg/kg test compound (S)-1-[(S)-1-({4-[(1H-Imidazol-2-yl)methyl]-1-piperidyl}carbonyl)-3-methylbutyl]-3-isobutyl-2-piperazinone. The test compound was dissolved in 0.5% methylcellulose with tween 80. Treatment started at a mean tumor volume of 100 mm³ at study day 17. The experiment was finished at day 58 of the study because of large tumors in group A and unchanged outcome. Tumor diameters were determined twice weekly. Statistical analysis was performed with the software Graph Pad Prism, Vers. 5.02 by using 2-Way-ANOVA with Bonferroni posttest. FIG. 1 shows tumor growth inhibition for the vehicle and the test compound.

### Example 320: Colony Formation Assay (CFA) with a Cervical Cancer Cell Line

Cell culture. CaSki cells (cervical cancer) were obtained from CLS GmbH, Eppelheim, Germany (cat #300145) and subcultured in ready-to-use RPMI1640 Media (CLS #820700) after addition of “Antibiotic Antimycotic Solution” (Sigma-Aldrich, St Louis, USA) at 1:100 dilution. The cells were expanded, and aliquots kept frozen in liquid nitrogen according to manufacturer's instructions; tested for mycoplasma contamination and genotyped (Microsynth AG, Balgach, Switzerland). Once thawed, aliquots were passaged every second or third day at a seed density of 20′000 cells/cm²; and used for a maximum of twenty passages. For passaging, cell culture flasks were rinsed twice with Ca++/Mg++-free Phosphate-Buffered Saline (DPBS, CLS #860015) and incubated with Accutase (CLS #830100) for 15 min at 37° C. Cells were resuspended in a four-fold volume of ready-to-use RPMI, centrifuged at 300 g for ten minutes, the supernatant discarded and the cells resuspended in ready-to-use RPMI1640 by pipetting up and down five times with a serological pipette. Cell density was determined with a Via-1 Cassette (Chemometec, Allerod, Denmark) on a Nucleocounter NC 3000 (Chemometec). Where not stated, consumables and chemicals were purchased from well-known suppliers.

Preparation of the cells for CFA. Cell culture flasks were rinsed twice with DPBS and incubated with Accutase for 15 min at 37° C. Cells were resuspended in a four-fold volume of ready-to use RPMI, centrifuged at 300 g for ten minutes, the supernatant discarded and the cells resuspended in ready-to-use RPMI1640 by pipetting up and down ten times with a serological pipette. Cell density was determined with a Via-1 Cassette on a Nucleocounter NC 3000. The required amount of cells was first diluted 1:10 in ready-to-use RPMI; cells pipetted five times up-down with a serological pipette; and the 1:10 solution added to the final volume needed for the whole assay setup. Cells were mixed again by pipetting up and down twenty times with a serological pipette. The cell density used for the experiment was of 140 cells/ml.

Seeding. Cells were seeded row-wise in 12-well plates, while triplicates were treated column-wise (i.e. all the first rows were seeded first, then the second rows, and finally the third rows). One and a half ml cell suspension solution (a total of 210 cells) were added per well.

Treatment. Compound stock solutions were prepared at a concentration of 30 mM in 50% DMSO/50% water; and diluted in the same solution so that the volume added to the wells was of 2.5 μl and the final DMSO well concentration of 0.08%. Untreated cells (negative controls) were incubated in a) medium only; and b) 0.08% DMSO. Test performance was monitored by a standard treatment with a fixed concentration of a reference compound that resulted in ˜80-90% inhibition of colony formation. The plates were swirled gently after addition of the compounds and the cells incubated for nine days at 37° C. Culture incubation solutions were replaced after five days.

Staining. Colonies were washed twice with ice-cold DPBS and fixed on ice with 1 ml ice-cold 10% methanol solution for 30 min. The methanol solution was removed and colonies incubated with 0.1% Crystal Violet/DBPS for 20 min at room temperature. The wells were rinsed consecutively with 1.5, 1, and 0.5 ml water at room temperature and let dry.

Destaining and data evaluation. Colony-bound crystal violet was solubilized in 500 μl 10% Acetic Acid. Plates were shaked for 30 sec and the acetic acid solution was transferred to a 2 ml well in a 96-deep-well plate. Five-hundred microliter 10% acetic acid were added again to each well of the 12-well plates, the plates shaken for 30 sec and the acetic acid transferred to the same well of the 96-deep-well plate, mixed well and 100 μl transferred to a clear-bottom 96-Well plate. Absorbance was measured at 600 nm with a SpectraMax M2e reader (Molecular Devices, Sunnyvale, CA, USA). Percentage of colony formation was calculated from the average of the well triplicates compared to the untreated DMSO controls after subtraction of blank values. Blanks were obtained from wells incubated for nine days with ready-to-use RPMI1640 only and processed the same way as colony-containing wells. A typical result is shown in TABLE 4 for four cell lines and four different compounds.

TABLE 4
Shows the GI50 (50% growth inhibition) concentrations (nM = nmol/lt)
for Compound 258; Compound 287; Compound 279; Compound 253;
and Compound 284, in a 9-day colony survival assay of cancer cell
lines derived from prostate (PC3); lung (A549); cervix (CaSki);
and colon (HCT116).

| Compound | PC3 | A549 | CaSki | HCT116 |
|---|---|---|---|---|
| Compound 258 | 3 | 5 | 2 | 1.5 |
| Compound 287 | 7 | 20 | 13 | 1.5 |
| Compound 279 | 1.5 | 5 | 4 | 1 |
| Compound 253 | 4 | 7 | 4.5 | 2 |
| Compound 284 | 3 | 10 | 6 | 1.5 |

### Example 321: Cell Proliferation Screening with a Sixty-Seven Cancer Cell Line Panel

For screening using a proliferation assay, the cells used were cultured at 37° C. with 5% CO2, except for those being cultured with L-15 medium (37° C. and 100% air, TABLE 7). Compound stock solutions (10 mM) were made in sterile water, aliquoted and stored at room temperature. Cisplatin was used as reference Control. Plastic consumables, culture media and supplements were purchased from well-known suppliers (TABLE 8).

Cell seeding (day −1). When necessary, cells were trypsinized using standard methods (see e.g. example 01). Cells were collected and resuspended in 5-6 mL of appropriate culture medium, counted and diluted to the needed density. Fifty-four μl (microliter) cells were seeded per well in a 384-well plate. Extra four wells per cell line were seeded on an additional plate for Day 0 reading (baseline cell density). Cells were incubated at 37° C. overnight.

Compound treatment and Day 0 reading (day 0). Tenfold final concentration of test compounds and Cisplatin were prepared in cell culture media (work dilutions). Six μl of work dilution solutions were dispensed into the corresponding wells in 384-well plates to bring the total volume up to 60 μl. Conditions were tested in triplicate. The plates were then incubated at 37° C. for five days. For the day 0 reading, 30 μl of CTG and 6 μl cell culture medium were added to the day 0 plates, contents mixed for 2 min on a plate shaker, and the plates incubated for 10 min at room temperature in the dark. Luminescence was recorded on an EnVision Multi Label Reader (2104-0010A, PerkinElmer, USA).

Endpoint CTG reading (day 5). The amounts of cells in the plates was determined by endpoint CTG-test. Thirty μl of CTG were added to the 60 μl of cell culture per well, contents mixed for 2 min on a plate shaker, and the plates incubated for 10 min at room temperature in the dark. Luminescence was recorded on an EnVision Multi Label Reader.

Data analysis. Fifty percent growth inhibition concentrations (GI50) were calculated based on percentage of control data (untreated cells) from each cell line (TABLE 5 (this example) and TABLE 6). Curves were fitted using a nonlinear regression model with a sigmoidal dose response. Examples of obtained curves are depicted in FIG. 2A-2F.

TABLE 5
Compound 258 GI50 for sixty-seven tested cell lines. Shows the GI50
(50% growth inhibition) concentration (nM = nmol/lt) for Compound
258 in a 5-day cell proliferation assay of 67 cancer cell lines
derived from 15 different tissues/organs. Cells with a value
“>10,000” mean that GI50 is higher than the highest
tested concentration (10 μM).

| Tumor Type | Cell Line | GI50 (nM) |
|---|---|---|
| Lymphoma | Granta-519 | 38 |
| | KARPAS-422 | 48 |
| | KARPAS-299 | 5 |
| | Ramos | 27 |
| | Daudi | 80 |
| | Raji | 28 |
| Leukemia | MOLM-13 | 4 |
| | HL-60 | 5 |
| | Kasumi-1 | 19 |
| | Jurkat | 18 |
| | MOLT-4 | 9 |
| | K562 | 17 |
| | U937 | 22 |
| | HS-5 | 5 |
| Brain/Nerves | H4 | 11 |
| | SF268 | 30 |
| Breast | MCF7 | 55 |
| | BT-20 | 15 |
| | BT474 | 72 |
| | SK-BR-3 | 12 |
| | AU565 | 3 |
| Cervix | CaSki | 35 |
| | SiHa | 52 |
| | MS751 | 51 |
| | DoTc2 4510 | 14 |
| | HT-3 | 330 |
| Ovary | OVCAR-3 | 44 |
| | OVCAR-4 | 12 |
| Colorectal | Caco-2 | 10 |
| | HCT116 | 5 |
| | HT-29 | 4 |
| | SW480 | 22 |
| | SW48 | 9 |
| | SW948 | 10 |
| | SW620 | 4 |
| Stomach/Gastric | MKN-45 | 4 |
| | IM95m | 4 |
| | MKN-1 | 2 |
| | HS 746.T | 8 |
| | SNU-16 | 13 |
| | SNU-5 | 4 |
| Kidney | 786-O | 28 |
| | Caki 2 | 14 |
| Liver | HEP-3B | 23 |
| | Hep G2 | 6 |
| | HUH-7 | 10 |
| Lung | A549 | 10 |
| | HCC4006 | 8 |
| | H460 | 11 |
| | HCC2935 | >10,000 |
| | MRC-5 | 12 |
| Oesophagus | KYSE-150 | 11 |
| | KYSE-270 | 44 |
| Pancreas | MIA PaCa-2 | 11 |
| | PANC-1 | 22 |
| Prostate | LNCaP-FGC | 2 |
| | DU145 | 7 |
| | 22RV1 | 9 |
| | PC-3 | 13 |
| | RPWE-1 | 4 |
| | VCAP | 128 |
| Skin | WM-266-4 | 41 |
| | SK-MEL28 | 50 |
| | SK-MEL5 | 51 |
| | A375 | 6 |
| | Malme-3M | >10,000 |
| | HDFA (fibroblast) | >10,000 |

TABLE 6
Cell proliferation GI50 (nM) of Compound 279, Compound 258
and Compound 284. Shows the GI50 (50% growth inhibition)
concentration (nM = nmol/lt) for Compound 258; Compound
279; and Compound 284 in a 5-day cell proliferation assay
of twelve cancer cell lines derived from six different
tissues/organs. Cells with a value “>10,000” mean
that GI50 is higher than the highest tested
concentration (10 μM).

| | | Compound | Compound | Compound |
|---|---|---|---|---|
| Tissue/Organ | Cell line | 279 | 258 | 284 |
| Skin (fibroblast) | HDFA | >10,000 | >10,000 | >10,000 |
| Colorectal | HCT 116 | 3 | 5 | 9 |
| | HS 746.T | 5 | 8 | 14 |
| | HT-29 | 4 | 4 | 7 |
| | SW48 | 6 | 9 | 14 |
| | SW620 | 2 | 4 | 7 |
| | SW948 | 3 | 10 | 12 |
| Leukemia | HS-5 | 3 | 5 | 10 |
| Lung | MRC-5 | 10 | 12 | 25 |
| Prostate | RWPE-1 | 3 | 4 | 10 |
| Stomach | SNU-16 | 10 | 13 | 22 |
| | SNU-5 | 3 | 4 | 6 |

TABLE 7
Example cell culture media for human
cancer cell lines used in the proliferation assay.

| Cell Line | Tissue Origin/Growth property | Culture medium | |
|---|---|---|---|
| HL-60 | Blood/Suspension | RPMI-1640 + | 10% FBS |
| Kasumi-1 | Blood/Suspension | RPMI-1640 + | 20% FBS |
| Granta-519 | Blood/Suspension | DMEM + | 10% FBS |
| H4 | Brain&Nerves/Adherent | RPMI1640 + | 10% FBS + Glutamax |
| BT-20 | Breast/Adherent | MEM + | 0.01 mM NEAA + |
| | | 10% FBS | |
| MCF7 | Breast/Adherent | MEM + | 0.01 mM NEAA + |
| | | 10% FBS + | 10 μg/mL Insulin |
| SK-BR-3 | Breast/Adherent | McCoy's 5a + | 10% FBS |
| Caco-2 | Colorectum/Adherent | MEM + | 0.01 mM NEAA + |
| | | 10% FBS | |
| SW480 | Colorectum/Adherent | L-15 + | 10% FBS |
| KYSE-270 | Esophagus/Adherent | RPMI-1640/F12 + | 2% FBS |
| Caki2 | Kidney/Adherent | McCoy's 5a + | 10% FBS |
| A549 | Lung/Adherent | Ham's F12K + | 10% FBS |
| OVCAR3 | Ovary/Adherent | RPMI1640 + | 20% FBS + 10 |
| | | μg/ml Insulin | |
| MIA PaCa-2 | Pancreas/Adherent | DMEM + | 10% FBS + 2.5% HS |

TABLE 8
Reagent suppliers.

| Reagent | Supplier | Cat# |
|---|---|---|
| F-12K | GIBCO | 21127022 |
| RPMI1640 | GIBCO | C22400500BT |
| DMEM | Hyclone | SH30243.01 |
| McCoy's 5A | GIBCO | 12330-031 |
| MEM | Hyclone | SH30024.01 |
| IMDM | GIBCO | 31980-030 |
| FBS | ExCell | FND500 |
| L-15 | GIBCO | 11415-064 |
| NEAA | GIBCO | 11140-050 |
| L-Glutamine 200 mM | GIBCO | 25030-081 |
| Trypsin (0.25%) | Hyclone | SH30042.02 |
| Insulin | Sigma | 11070-73-8 |
| GlutaMAX | GIBCO | 35050-061 |
| DMSO | Amresco | 0231 |
| 384 well cell culture plate | Corning | 3765 |
| CellTiter-Glo Luminescent Cell | Promega | G7573 |
| Viability kit (CTG) | | |

### Example 322: Castration Resistant Prostate Cancer Patient-Derived Xenograft

NSG NOD.Cg-PrkdcscidIj2rgtm1Wjl/SzJ mice (6-8 weeks of age) were injected with patient-derived castration-resistant prostate tumor cells (model #PR6511, Crown Bioscience UK Ltd Hillcrest, Osgathorpe Leicestershire, UK). The cells were inoculated subcutaneously into the left flank of 40 male NSG mice. When tumors reached a mean volume of approximately 100 mm³ (i.e. 100 mm³+/−54 mm³, FIG. 3), mice were randomly assigned to five treatment groups of each eight mice (TABLE 9). Body weights were measured three times weekly prior to the initiation of dosing and daily thereafter. Tumor burden was assessed by caliper measurement three times weekly. Dosing breaks were applied when tolerability issues arose (body weight loss>10). Upon completion of the scheduled dosing phase at day 57, tumor outgrowth was monitored for groups 3, 4 and w up to study day 77. Groups 1 and 2 were terminated on study day 57. Results of the experiment are shown in TABLES 10 and 11; and FIGS. 3 and 4. The experiment was performed in the Crown Bioscience Ltd. testing facility (Crown Bioscience UK Ltd Hillcrest, Dodgeford Lane, Osgathorpe Leicestershire LE12 9TE, UK).

TABLE 9
CRPC PDX mice treatment group assignment.

| | | | Treatment |
|---|---|---|---|
| Group | Compound | Formulation | schedule |
| 1 | Vehicle | 0.5% Methylcellulose + | p.o. QD |
| | | 0.1% Tween-80 | |
| 2 | Enzalutamide | 1% w/v Carboxymethyl | p.o. QD, 30 mg/kg |
| | | Cellulose (CMC; low | |
| | | viscosity); 0.1% v/v | |
| | | Tween-80; 5% v/v DMSO | |
| 3 | Compound | 0.5% Methylcellulose + | p.o. QD, 10 mg/kg, |
| | 258 | 0.1% Tween-80 | 3 mg/kg from day |
| | | | 51 on |
| 4 | Compound | 0.5% Methylcellulose + | p.o. QD, 6 mg/kg |
| | 258 | 0.1% Tween-80 | |
| 5 | Compound | 0.5% Methylcellulose + | p.o. QD, 6 mg/kg |
| | 284 | 0.1% Tween-80 | |

p.o. = oral administration (oral gavage).
QD = once a day.
v/v = volume/volume

TABLE 10
CRPC PDX RESPONSE SUMMARY.

| Treatment (group) | PR | CR | TFS |
|---|---|---|---|
| Vehicle (1) | 0 | 0 | 0 |
| Enzalutamide (2) | 0 | 0 | 0 |
| Compound 258 10 mg/kg (3) | 2 | 4 | 3 |
| Compound 258 6 mg/kg (4) | 2 | 3 | 3 |
| Compound 284 6 mg/kg (5) | 2 | 3 | 3 |

PR (partial regression) = number of mice presenting a tumor size ≤ 50% lower than initial tumor size during at least 3 consecutive measurements and ≥ 13.5 mm³ for one or more of these three measurements;
CR (complete regression) = number of mice presenting ≤ 13.5 mm³ tumor size during at least 3 consecutive measurements;
TFS (Tumor Free Survival) = number of complete regressions recorded up to Group Day End.
Animals were scored only once during the study for a PR or CR event and only as CR if both PR and CR criteria were satisfied.

TABLE 11
CRPC PDX TWO-WAY ANOVA (tumor
size at the end of the study).

| | | Compound | Compound | Compound |
|---|---|---|---|---|
| | Enzalut- | 258 | 258 | 284 |
| vs. | amide | 10 mg/kg | 6 mg/kg | 6 mg/kg |
| Vehicle | n.s. | p < 0.0001 | p < 0.0001 | p < 0.0001 |
| Enzalutamide | — | p < 0.0001 | p < 0.0001 | p < 0.0001 |
| Compound 258 | — | — | n.s. | n.s. |
| 10 mg/kg | | | | |
| Compound 258 | — | — | — | n.s. |
| 6 mg/kg | | | | |
| Compound 284 | — | — | — | — |
| 6 mg/kg | | | | |

n.s. = not significant.

### Example 323: Cell-Derived Xenograft Mouse Model for Prostate Cancer

Male BALB/c nude mice (7-9 weeks of age) were injected with hormone-refractory DU-145 human prostate cancer cells. The cells were inoculated subcutaneously into the left flank of 48 mice. When tumors reached a mean volume of approximately 105 mm³, mice were randomly assigned to eight treatment groups of each six mice (TABLE 12). Body weights were measured three times weekly prior to the initiation of dosing and daily thereafter. Tumor burden was assessed by caliper measurement three times weekly. Dosing breaks were applied when tolerability issues arose (body weight loss>10%). The study was terminated 4 weeks post first dosing. Results of the experiment are shown in TABLE 13, FIG. 5 and FIG. 6. The experiment was performed in the Crown Bioscience Ltd. (Beijing) testing facility (Ground Floor, Light Muller Building, Changping Sector of Zhongguancun Scientific Park, No. 21 Huoju Road, Changping District, Beijing, CHIN).

TABLE 12
DU-145 CDX mice treatment group assignment.

| | | | Treatment |
|---|---|---|---|
| Group | Compound | Formulation | schedule |
| 1 | Vehicle | 0.5% Methylcellulose + 0.1% | p.o. QD |
| | | Tween-80 | |
| 2 | Compound | 0.5% Methylcellulose + 0.1% | p.o. QD, |
| | 258 | Tween-80 | 10 mg/kg |
| 3 | Compound | 0.5% Methylcellulose + 0.1% | p.o. QD, 6 |
| | 258 | Tween-80 | mg/kg |
| 4 | Compound | 0.5% Methylcellulose + 0.1% | p.o. QD, 3 |
| | 258 | Tween-80 | mg/kg |
| 5 | Compound | 0.5% Methylcellulose + 0.1% | p.o. QD, 1 |
| | 258 | Tween-80 | mg/kg |
| 6 | Compound | 0.5% Methylcellulose + 0.1% | p.o. QD, 3 |
| | 279 | Tween-80 | mg/kg |
| 7 | Compound | 0.5% Methylcellulose + 0.1% | p.o. QD, 3 |
| | 253 | Tween-80 | mg/kg |
| 8 | Compound | 0.5% Methylcellulose + 0.1% | p.o. QD, 3 |
| | 284 | Tween-80 | mg/kg |

p.o. = oral administration (oral gavage).
QD = once a day.

TABLE 13
Test compound antitumoral activity on subcutaneous DU-
145 prostate cancer CDX model in Male BALB/c Nude Mice.

| | | Tumor Size | T/C | P |
|---|---|---|---|---|
| Group | Treatment Description | (mm³)^{a} day 35 | (%) | value^{b} |
| 1 | Vehicle | 693 ± 116 | — | — |
| 2 | Compound 258, 10 mg/kg, | 47 ± 4 | 6.8 | 0.003 |
| | QD × 4 weeks | | | |
| 3 | Compound 258, 6 mg/kg, | 32 ± 2 | 4.6 | 0.002 |
| | QD × 4 weeks | | | |
| 4 | Compound 258, 3 mg/kg, | 105 ± 25 | 15.2 | 0.003 |
| | QD × 4 weeks | | | |
| 5 | Compound 258, 1 mg/kg, | 378 ± 27 | 54.5 | 0.4 |
| | QD × 4 weeks | | | |
| 6 | Compound 279, 3 mg/kg, | 516 ± 59 | 74.5 | 0.204 |
| | QD × 4 weeks | | | |
| 7 | Compound 253, 3 mg/kg, | 480 ± 49 | 69.3 | 0.120 |
| | QD × 4 weeks | | | |
| 8 | Compound 284, 3 mg/kg, | 347 ± 48 | 50.1 | 0.02 |
| | QD × 4 weeks | | | |

^{a}Mean ± SEM;
^{b}vs. vehicle control (T-test);
T/C = tumor-to-control ratio (volume/volume)

### Example 324: Cell-Derived Xenograft Mouse Model for Colorectal Cancer

Female BALB/c nude mice (8-9 weeks of age) were injected with HCT116 human colon cancer cells. The cells were inoculated subcutaneously into the left flank of 48 mice. When tumors reached a mean volume of approximately 100 mm³, mice were randomly assigned to eight treatment groups of each six mice (TABLE 14). Body weights were measured three times weekly prior to the initiation of dosing and daily thereafter. Tumor burden was assessed by caliper measurement twice weekly. Dosing breaks were applied when tolerability issues arose (body weight loss>10%). The study was terminated four weeks post first dosing. Results of the experiment are shown in TABLE 15, FIG. 7 and FIG. 8. The experiment was performed in the Crown Bioscience Ltd. (Beijing) testing facility (Ground Floor, Light Muller Building, Changping Sector of Zhongguancun Scientific Park, No. 21 Huoju Road, Changping District, Beijing, CHN).

TABLE 14
HCT116 CDX mice treatment group assignment.

| | | | Treatment |
|---|---|---|---|
| Group | Compound | Formulation | schedule |
| 1 | Vehicle | 0.5% Methylcellulose + 0.1% | p.o. QD |
| | | Tween-80 | |
| 2 | Avastin | PBS | i.v. BIW, |
| | | | 10 mg/kg |
| 3 | Compound | 0.5% Methylcellulose + 0.1% | p.o. BIW, |
| | 258 | Tween-80 | 20 mg/kg |
| 4 | Compound | 0.5% Methylcellulose + 0.1% | p.o. QD, 6 |
| | 258 | Tween-80 | mg/kg |
| 5 | Compound | 0.5% Methylcellulose + 0.1% | p.o. QD, 3 |
| | 258 | Tween-80 | mg/kg |
| 6 | Compound | 0.5% Methylcellulose + 0.1% | p.o. QD, 6 |
| | 279 | Tween-80 | mg/kg |
| 7 | Compound | 0.5% Methylcellulose + 0.1% | p.o. QD, 6 |
| | 284 | Tween-80 | mg/kg |
| 8 | Compound | 0.5% Methylcellulose + 0.1% | p.o. QD, 3 |
| | 284 | Tween-80 | mg/kg |

p.o. = oral administration (oral gavage).
i.v. = intravenous administration.
QD = once a day.
BIW = twice a week.

TABLE 15
Test compound antitumoral activity on subcutaneous HCT116
colorectal cancer CDX model in female BALB/c nude mice.

| | | Tumor Size | T/C | P |
|---|---|---|---|---|
| Group | Treatment Description | (mm³)^{a} day 37 | (%) | value^{b} |
| 1 | Vehicle | 1187.9 ± 154.3 | — | — |
| 2 | Avastin, 10 mg/kg, BIW | 719.9 ± 93.9 | 60.6 | 0.027 |
| 3 | Compound 258, 20 mg/kg, | 696.8 ± 93.7 | 58.7 | 0.022 |
| | BIW | | | |
| 4 | Compound 258, 6 mg/kg, QD | 253.2 ± 39.2 | 21.3 | <0.001 |
| 5 | Compound 258, 3 mg/kg, QD | 436.2 ± 58.9 | 36.7 | 0.001 |
| 6 | Compound 279, 6 mg/kg, QD | 1064.0 ± 141.1 | 89.6 | 0.567 |
| 7 | Compound 284, 6 mg/kg, QD | 942.0 ± 114.9 | 79.3 | 0.230 |
| 8 | Compound 284, 3 mg/kg, QD | 827.4 ± 125.7 | 69.7 | 0.100 |

^{a}Mean ± SEM;
^{b}vs. vehicle control (T-test);
T/C = tumor-to-control ratio (volume/volume)
Group-2 vs. Group-3, p = 0.865;
Group-2 vs. Group-4, p = 0.001;
Group-2 vs. Group-5, p = 0.028;
Group-2 vs. Group-6, p = 0.070;
Group-2 vs. Group-7, p = 0.165;
Group-2 vs. Group-8, p = 0.509;

### Example 325: Cell-Derived Xenograft Mouse Model for Gastric Cancer

Female BALB/c nude mice (8-9 weeks of age) were injected with MKN45 human gastric adenocarcinoma cells. The cells were inoculated subcutaneously into the left flank of 48 mice. When tumors reached a mean volume of approximately 103 mm³, mice were randomly assigned to eight treatment groups of each six mice (TABLE 16). Body weights were measured three times weekly prior to the initiation of dosing and daily thereafter. Tumor burden was assessed by caliper measurement twice weekly. Dosing breaks were applied when tolerability issues arose (body weight loss>10%). The study was terminated four weeks post first dosing. Results of the experiment are shown in TABLE 17, FIG. 9 and FIG. 10. The experiment was performed in the Crown Bioscience Ltd. (Beijing) testing facility (Ground Floor, Light Muller Building, Changping Sector of Zhongguancun Scientific Park, No. 21 Huoju Road, Changping District, Beijing, CHN).

TABLE 16
MKN45 CDX mice treatment group assignment.

| | | | Treatment |
|---|---|---|---|
| Group | Compound | Formulation | schedule |
| 1 | Vehicle | 0.5% Methylcellulose + 0.1% | p.o. QD |
| | | Tween-80 | |
| 2 | Paclitaxel | PBS | i.v. QW, 12 |
| | | | mg/kg |
| 3 | Compound | 0.5% Methylcellulose + 0.1% | p.o. BIW, 20 |
| | 258 | Tween-80 | mg/kg |
| 4 | Compound | 0.5% Methylcellulose + 0.1% | p.o. QD, 6 |
| | 258 | Tween-80 | mg/kg |
| 5 | Compound | 0.5% Methylcellulose + 0.1% | p.o. QD, 3 |
| | 258 | Tween-80 | mg/kg |
| 6 | Compound | 0.5% Methylcellulose + 0.1% | p.o. QD, 6 |
| | 253 | Tween-80 | mg/kg |
| 7 | Compound | 0.5% Methylcellulose + 0.1% | p.o. QD, 6 |
| | 284 | Tween-80 | mg/kg |
| 8 | Compound | 0.5% Methylcellulose + 0.1% | p.o. QD, 3 |
| | 284 | Tween-80 | mg/kg |

p.o. = oral administration (oral gavage).
i.v. = intravenous administration.
QD = once a day.
QW = once a week.

TABLE 17
Test compound antitumoral activity on subcutaneous MKN45
gastric cancer CDX model in female BALB/c nude mice.

| | | Tumor Size | T/C | P |
|---|---|---|---|---|
| Group | Treatment Description | (mm³)^{a} day 29 | (%) | value^{b} |
| 1 | Vehicle | 1189.6 ± 139.6 | — | — |
| 2 | Paclitaxel, 12 mg/kg, QW | 554.9 ± 52.8 | 46.6 | 0.002 |
| 3 | Compound 258, 20 mg/kg, | 763.4 ± 84.5 | 64.2 | 0.026 |
| | BIW | | | |
| 4 | Compound 258, 6 mg/kg, QD | 186.9 ± 17.3 | 15.7 | <0.001 |
| 5 | Compound 258, 3 mg/kg, QD | 428.0 ± 52.5 | 36.0 | <0.001 |
| 6 | Compound 253, 6 mg/kg, QD | 506.5 ± 71.0 | 42.6 | 0.001 |
| 7 | Compound 284, 6 mg/kg, QD | 592.6 ± 109.7 | 49.8 | 0.007 |
| 8 | Compound 284, 3 mg/kg, QD | 709.0 ± 55.6 | 59.6 | 0.010 |

^{a}Mean ± SEM;
^{b}vs. vehicle control (T-test);
T/C = tumor-to-control ratio (volume/volume)
Group-2 vs. Group-3, p = 0.063;
Group-2 vs. Group-4, p < 0.001;
Group-2 vs. Group-5, p = 0.119;
Group-2 vs. Group-6, p = 0.596;
Group-2 vs. Group-7, p = 0.764;
Group-2 vs. Group-8, p = 0.072

### Example 326: Cell-Derived Xenograft Mouse Model for Cervical Cancer

Female BALB/c nude mice (7-9 weeks of age) were injected with SiHa human HPV16-positive (Human Papillomavirus type 16) cervical cancer cells. The cells were inoculated subcutaneously into the left flank of 48 mice. When tumors reached a mean volume of approximately 111 mm³, mice were randomly assigned to eight treatment groups of each six mice (TABLE 18). Body weights were measured three times weekly prior to the initiation of dosing and daily thereafter. Tumor burden was assessed by caliper measurement twice weekly. Dosing breaks were applied when tolerability issues arose (body weight loss>10%). The study was terminated four weeks post first dosing. Results of the experiment are shown in TABLE 19, FIG. 11 and FIG. 12. The experiment was performed in the Crown Bioscience Ltd. (Beijing) testing facility (Ground Floor, Light Muller Building, Changping Sector of Zhongguancun Scientific Park, No. 21 Huoju Road, Changping District, Beijing, CHIN).

TABLE 18
SiHa CDX mice treatment group assignment.

| | | | Treatment |
|---|---|---|---|
| Group | Compound | Formulation | schedule |
| 1 | Vehicle | 0.5% Methylcellulose + | p.o. BID |
| | | 0.1% Tween-80 | |
| 2 | Compound | 0.5% Methylcellulose + | p.o. BID, |
| | 248 | 0.1% Tween-80 | 1 mg/kg |
| 3 | Compound | 0.5% Methylcellulose + | p.o. BID, |
| | 248 | 0.1% Tween-80 | 3 mg/kg |
| 4 | Compound | 0.5% Methylcellulose + | p.o. QD, |
| | 248 | 0.1% Tween-80 | 6 mg/kg |
| 5 | Compound | 0.5% Methylcellulose + | p.o. BID, |
| | 258 | 0.1% Tween-80 | 1 mg/kg |
| 6 | Compound | 0.5% Methylcellulose + | p.o. BID, |
| | 258 | 0.1% Tween-80 | 3 mg/kg, |
| | | | p.o. QD from |
| | | | treatment |
| | | | day 7 on |
| 7 | Compound | 0.5% Methylcellulose + | p.o. BID, |
| | 273 | 0.1% Tween-80 | 3 mg/kg |
| 8 | Compound | 0.5% Methylcellulose + | p.o. BID, |
| | 318 | 0.1% Tween-80 | 3 mg/kg |

p.o. = oral administration (oral gavage).
QD = once a day.
BID = twice a day.

TABLE 19
Test compound antitumoral activity on subcutaneous HPV16-positive
SiHa cervical cancer CDX model in female BALB/c nude mice.

| | | Tumor Size | T/C | P |
|---|---|---|---|---|
| Group | Treatment Description | (mm³)^{a} day 37 | (%) | value^{b} |
| 1 | Vehicle | 936 ± 89 | — | — |
| 2 | Compound 248, 1 mg/kg, BID | 586 ± 82 | 62.6 | 0.016 |
| 3 | Compound 248, 3 mg/kg, BID | 178 ± 27 | 19 | <0.001 |
| 4 | Compound 248, 6 mg/kg, QD | 238 ± 37 | 25.4 | <0.001 |
| 5 | Compound 258, 1 mg/kg, BID | 296 ± 27 | 31.6 | <0.001 |
| 6 | Compound 258, 3 mg/kg, | 104 ± 30 | 11.1 | <0.001 |
| | BID/QD | | | |
| 7 | Compound 273, 3 mg/kg, BID | 942.0 ± 114.9 | 79.3 | 0.230 |
| 8 | Compound 318, 3 mg/kg, BID | 351 ± 68 | 37.5 | <0.001 |

^{a}Mean ± SEM;
^{b}vs. vehicle control (T-test);
T/C = tumor-to-control ratio (volume/volume)

### Example 327: Cell-Derived Xenograft Mouse Model for Acute Myeloid Leukemia Tumor Spread (Bone Marrow Engraftment)

Twenty-four female NOD-SCID mice (NOD. CB 17-Prkdc^{scid}/J, 4-5 weeks of age) were pretreated for two days once daily i.p. with 100 mg/kg cyclophosphamide in order to reduce the endogenous bone marrow population and to facilitate bone marrow engraftment of MOLM13-Luc cells, an acute myeloid leukemia cell line transduced using a plasmid encoding a luciferase-neomycin fusion protein (cell line identifier #200, Proqinase GmbH, Freiburg, Germany). Forty-eight hours after the last cyclophosphamide treatment, one million MOLM13-Luc cells in 100 μl 0.9% NaCl were intravenously implanted into the animals. In the following study period, the growth of the MOLM13-Luc cells was monitored on days 4, 8, 11, 15 and 19 using in vivo bioluminescence imaging. On day 8, animals were randomly assigned to three treatment groups of six mice each (TABLE 20) and treatment was initiated for all groups on the same day. The study was terminated 19 days post first dosing. Animals were weighed and euthanized by cervical dislocation. Selected organs (femur, lumbar spine, lymph nodes (inguinal and axillary) and peritoneal carcinomatosis samples from fatty tissues) were collected, weighed, appropriately processed and the luciferase activity of the homogenates measured using an ex vivo luciferase assay (#E1501, Promega, Madison, WI, USA) according to the instructions from the manufacturer. The luciferase activity was read with an Enspire Reader (Perkin Elmer, Waltham, MA, USA). Except for lymph nodes, organ weights were determined during necropsy in order to normalize luciferase activities. Results of the experiment are shown in FIGS. 13-15. The experiment was performed in the Proqinase animal test facility (Proqinase GmbH, D-79106 Freiburg, Germany).

TABLE 20
MOLM13-Luc CDX mice treatment group assignment.

| | | | Treatment |
|---|---|---|---|
| Group | Compound | Formulation | schedule |
| 1 | Vehicle | 0.5% Methylcellulose + 0.1% | p.o. QD |
| | | Tween-80 | |
| 2 | Compound | 0.5% Methylcellulose + 0.1% | p.o. QD, |
| | 258 | Tween-80 | 1 mg/kg |
| 3 | Compound | 0.5% Methylcellulose + 0.1% | p.o. QD, |
| | 258 | Tween-80 | 3 mg/kg |
| 4 | Compound | 0.5% Methylcellulose + 0.1% | p.o. QD, |
| | 258 | Tween-80 | 6 mg/kg |

p.o. = oral administration (oral gavage).
QD = once a day.

### Example 328: Patient-Derived Xenograft Mouse Model for Head-and-Neck Squamous Cell Carcinoma

Female NMRI nu/nu mice were injected with cells derived from a human head-and-neck cancer (model #HN10309, an HPV-positive head-and-neck squamous cell carcinoma, EPO-GmbH, Berlin, Germany). The cells were inoculated subcutaneously into the left flank of 24 mice. Thirty-three days later tumors reached a mean volume of approximately 116 mm³, and mice were randomly assigned to three treatment groups of each eight mice (TABLE 21). Body weights were measured twice weekly. Tumor burden was assessed by caliper measurement twice weekly. Dosing breaks were applied when tolerability issues arose (body weight loss>0%). The study was terminated four weeks post first dosing. Results of the experiment are shown in TABLE 22, FIG. 16 and FIG. 17. The experiment was performed in the EPO animal testing facility (Experimental Pharmacology and Oncology GmbH, 13125 Berlin-Buch, Germany).

TABLE 21
HNSCC PDX mice treatment group assignment.

| | | | Treatment |
|---|---|---|---|
| Group | Compound | Formulation | schedule |
| A | Vehicle | 0.5% Methylcellulose + | p.o. BID |
| | | 0.1% Tween-80 | |
| B | Compound | 0.5% Methylcellulose + | p.o. BID, |
| | 282 | 0.1% Tween-80 | 30 mg/kg |
| C | Compound | 0.5% Methylcellulose + | p.o. BID, |
| | 248 | 0.1% Tween-80 | 30 mg/kg |

p.o. = oral administration (oral gavage).
BID = twice a day.

TABLE 22
Test compound antitumoral activity on subcutaneous
HPV-positive HNSCC model in female NMRI nu/nu mice.

| | | Compound | | Compound | |
|---|---|---|---|---|---|
| Day | Vehicle | 282 | Significance | 248 | Significance |
| 33 | 0.1156 | 0.1161 | ns | 0.1159 | ns |
| 35 | 0.1499 | 0.1308 | ns | 0.1248 | ns |
| 38 | 0.2386 | 0.1751 | ns | 0.1389 | ns |
| 41 | 0.2908 | 0.2086 | ns | 0.1093 | * |
| 45 | 0.3670 | 0.3560 | ns | 0.1794 | * |
| 49 | 0.5079 | 0.4920 | ns | 0.2398 | *** |
| 53 | 0.6693 | 0.6364 | ns | 0.2964 | *** |
| 56 | 0.6875 | 0.6558 | ns | 0.2569 | *** |

Bonferroni posttests to vehicle group (mean TV values in cm³).
n.s not significant;
* = p < 0.05,
** = p < 0.01;
*** = p < 0.001.

### Example 329: Patient-Derived Xenograft Mouse Model for Head-and-Neck Squamous Cell Carcinoma

Female NMRI nu/nu mice were injected with cells derived from a human head-and-neck cancer (model #HN11303, an HPV-positive head-and-neck squamous cell carcinoma, EPO-GmbH, Berlin, Germany). The cells were inoculated subcutaneously into the left flank of 40 mice. Thirteen days later tumors reached a mean volume of approximately 107 mm³, and mice were randomly assigned to five treatment groups of each eight mice (TABLE 23). Body weights were measured twice weekly. Tumor burden was assessed by caliper measurement twice weekly. Dosing breaks were applied when tolerability issues arose (body weight loss>10). The study was terminated five weeks post first dosing. Results of the experiment are shown in TABLE 24, FIG. 18 and FIG. 19. The experiment was performed in the EPO animal testing facility (Experimental Pharmacology and Oncology GmbH, 13125 Berlin-Buch, Germany).

TABLE 23
HNSCC PDX mice treatment group assignment.

| Group | Compound | Formulation | Treatment schedule |
|---|---|---|---|
| A | Vehicle | 0.5% Methylcellulose + | i.p. BID |
| | | 0.1% Tween-80 | |
| B | Compound | 0.5% Methylcellulose + | i.p. BID, 30 mg/kg |
| | 57 | 0.1% Tween-80 | |
| C | Compound | 0.5% Methylcellulose + | i.p. BID, 30 mg/kg day 13-17 |
| | 248 | 0.1% Tween-80 | dosing break day 18-19 |
| | | | i.p. QD, 10 mg/kg day 20 |
| | | | i.p. QD, 3 mg/kg day 21-31 |
| | | | i.p. QD, 10 mg/kg day 32-47 |
| D | Compound | 0.5% Methylcellulose + | i.p. BID, 30 mg/kg |
| | 282 | 0.1% Tween-80 | |
| E | Compound | 0.5% Methylcellulose + | i.p. BID, 30 mg/kg day 13-27 |
| | 273 | 0.1% Tween-80 | dosing break day 28-31 |
| | | | i.p. QD, 10 mg/kg day 32-47 |

i.p. = intraperitoneal injection.
BID = twice a day.
QD = once a day.

TABLE 24
Test compound antitumoral activity on subcutaneous
HPV-positive HNSCC model in female NMRI nu/nu mice.

| | | Compound | | Compound | | Compound | | Compound | |
|---|---|---|---|---|---|---|---|---|---|
| Day | Vehicle | 57 | Sign. | 248 | Sign. | 282 | Sign. | 273 | Sign. |
| 13 | 0.1060 | 0.1073 | ns | 0.1064 | ns | 0.1073 | ns | 0.1063 | ns |
| 15 | 0.1286 | 0.1106 | ns | 0.0958 | ns | 0.1061 | ns | 0.1073 | ns |
| 18 | 0.1363 | 0.1166 | ns | 0.0916 | ns | 0.1040 | ns | 0.0948 | ns |
| 21 | 0.1566 | 0.1081 | ns | 0.0511 | *** | 0.0971 | * | 0.0875 | ** |
| 25 | 0.1650 | 0.1233 | ns | 0.0499 | *** | 0.1111 | ns | 0.0451 | *** |
| 29 | 0.1805 | 0.1421 | ns | 0.0288 | *** | 0.1030 | *** | 0.0194 | *** |
| 33 | 0.2081 | 0.2009 | ns | 0.0068 | *** | 0.1273 | *** | 0.0056 | *** |
| 36 | 0.1988 | 0.1776 | ns | 0.0041 | *** | 0.1185 | *** | 0.0031 | *** |

Bonferroni posttests to vehicle group (mean TV values in cm³).
ns not significant;
* = p < 0.05;
** = p < 0.01;
*** = p < 0.001.
According to RECIST guidelines (Response Evaluation Criteria In Solid Tumors; Eisenhauer et al., Eur. J. Cancer 45 (2009) 228-247), treatment outcome of all mice in group C (Compound 248) and E (Compound 273) was classified as complete remission.

### Example 330: Cell-Derived Syngeneic Mouse Model for Colorectal Carcinoma Combined with an Immuno-Oncology Treatment

Female BALB/c mice (6-8 weeks of age) were injected with cells derived from a syngeneic chemically induced colon cancer (CT-26 cells). The cells were inoculated subcutaneously into the left flank of 60 mice. After seven days, tumors reached a mean volume of approximately 100 mm³, and mice were randomly assigned to six treatment groups of each ten mice (TABLE 25). Body weights were measured daily until treatment start, then twice weekly. Tumor burden was assessed twice weekly by caliper measurement. The study was terminated four weeks post first dosing. Results of the experiment are shown in TABLE 26, FIG. 20 and FIG. 21. The experiment was performed in the Crown Bioscience Inc. (Beijing) animal testing facility (Ground Floor, Light Muller Building, Changping Sector of Zhongguancun Scientific Park, No. 21 Huoju Street, Changping District, Beijing, China, 102200).

TABLE 25
CT26 tumor model mice treatment group assignment.

| | | | Treatment |
|---|---|---|---|
| Group | Compound | Formulation | schedule |
| 1 | Vehicle | 0.5% Methylcellulose + | p.o. QD, |
| | Vehicle | 0.1% Tween-80 | i.p BIW |
| 2 | Vehicle | 0.5% Methylcellulose + | p.o. QD, |
| | Anti-PD-1* | 0.1% Tween-80 | i.p BIW, |
| | | | 10 mg/kg |
| 3 | Compound 258 | 0.5% Methylcellulose + | p.o. QD, |
| | Vehicle | 0.1% Tween-80 | 1 mg/kg |
| | | | i.p BIW |
| 4 | Compound 258 | 0.5% Methylcellulose + | p.o. QD, |
| | Vehicle | 0.1% Tween-80 | 3 mg/kg |
| | | | i.p BIW |
| 5 | Compound 258 | 0.5% Methylcellulose + | p.o. QD, |
| | Anti-PD-1* | 0.1% Tween-80 | 1 mg/kg |
| | | | i.p BIW, |
| | | | 10 mg/kg |
| 6 | Compound 258 | 0.5% Methylcellulose + | p.o. QD, |
| | Anti-PD-1* | 0.1% Tween-80 | 3 mg/kg |
| | | | i.p BIW, |
| | | | 10 mg/kg |

p.o. = oral administration (oral gavage).
BIW = twice a week.
QD = once a day.
*= RMP1-14 antibody.

TABLE 26
Survival data for test compound antitumoral activity on
subcutaneous syngeneic colon cancer model in female
BALB/c mice in combination with anti-PD1 antibodies
(mice alive between day 21 and day 36 of the experiment).

| | | | | | Compound | Compound |
|---|---|---|---|---|---|---|
| | | | Compound | Compound | 258 | 258 |
| | | Anti- | 258 | 258 | 1 mg/kg + | 3 mg/kg + |
| Day | Vehicle | PD1 | 1 mg/kg | 3 mg/kg | Anti-PD1 | Anti-PD1 |
| 21 | 10 | 10 | 10 | 10 | 10 | 10 |
| 23 | 9 | 10 | 10 | 8 | 10 | 10 |
| 25 | 7 | 7 | 5 | 6 | 10 | 10 |
| 28 | 2 | 5 | 2 | 2 | 7 | 9 |
| 30 | 1 | 5 | 1 | 0 | 6 | 8 |
| 32 | 0 | 3 | 0 | 0 | 4 | 4 |
| 34 | 0 | 3 | 0 | 0 | 4 | 4 |
| 36 | 0 | 3 | 0 | 0 | 4(*) | 4(*) |

(*)One mouse in group 5 (combination of Compound 258 1 mg/kg and anti-PD1 antibodies) and one mouse in group 6 (combination Compound 258 3 mg/kg and anti-PD1 antibodies) had a complete tumor regression (tumor volume not measurable).

### Example 331: P300/CBP—Androgen Receptor Target Gene Expression Regulation in Prostate Cancer Cells

LNCaP prostate cancer cells (CLS GmbH) were seeded at a density of 15′000 cells/cm² in 48-well cell culture-treated plates and cultured for 72 hours in RPMI1640 medium (Sigma-Aldrich) supplemented with Glutamax I (ThermoFisher-Gibco), “Antibiotic and Antimycotic Solution” (Sigma-Aldrich) and 1% fetal calf serum (Sigma-Aldrich). AR-driven gene expression response was induced by addition of the androgen signaling agonist dihydrotestosterone (Selleck Chemicals, Houston, TX, USA) to a concentration of 100 nanomol/Lt for 4 hours. Cells were treated with Compound 258 during dihydrotestosterone induction. Culture medium was carefully removed, cells were washed 1× with Phosphate-Buffered Saline (Sigma-Aldrich) and lysed using the SingleShot Cell Lysis Kit (Bio-Rad, Hercules, CA, USA). Gene expression of well-known AR-responsive genes prostate-specific antigen (KLK3, ThermoFisher), transmembrane serine protease 2 (TMPRSS2, ThermoFisher) and prostein (SLC45A3, ThermoFisher) was assessed by quantitative PCR after reverse transcription of the LNCaP RNA with the Applied Biosystem High-Capacity cDNA Reverse Transcription Kit (ThermoFisher). Gene expression was normalized against four reference genes (RPLP0, GUSB, GAPDH and ACTB, all probe detection systems were from Bio-Rad). Results of the experiment are shown in FIG. 22.

### Example 332: P300/CBP—Androgen Receptor-Dependent Protein Expression Regulation in a Castration-Resistant Prostate Cancer Patient-Derived Xenograft Mouse Model

Mice from groups 1, 3 and 4 of the CPRC prostate patient-derived xenograft model (TABLE 9, EXAMPLE 322) were used to analyze serum PSA at the end of the experiment. In groups 3 and 4, only serum from mice that had a detectable tumor was analyzed. PSA was detected by ELISA (Human Kallikrein 3/PSA Quantikine ELISA Kit, R&D Systems, Minneapolis, MN, USA) according to manufacturer's instructions. Minimal relative expected PSA-levels were calculated based on minimal PSA/tumor size ratio of vehicle-treated mice. Results of the analysis are presented in FIG. 22.

### Example 333: P300/CBP—Hypoxia Inducible Factor Alpha-Dependent Protein Expression Regulation in Two Gastric/Colorectal Cancer Cell-Derived Xenograft Mouse Model

Mice from groups 1, 2, 4 and 5 of the HCT-116 colorectal cancer model (TABLE 12, EXAMPLE 324) and MKN45 gastric cancer model (TABLE 14, EXAMPLE 325) were used to analyze tumor Vascular Endothelial Growth Factor A (VEGF) protein expression at the end of the experiment. Tumors homogenates were prepared using a 2× Lysis Buffer (RayBiotech Life, Norcross, GA, USA) according to manufacturer's instructions. VEGF was quantified in 50 micrograms tumor by ELISA (Human VEGF Quantikine ELISA Kit, R&D Systems) according to manufacturer's instructions. Results of the analysis are presented in FIG. 24.

### Example 334: P53 Protein Reactivation in an HPV16-Positive Cervical Cancer Tumor Cell Line

HPV16-positive cervical cancer cells CaSki were subcultured as described in EXAMPLE 1, seeded at a density of 20′000 cells/cm²; and readily treated with Compound 258 for 72 hrs. For western blot analysis, cells were removed from the cell culture vessels by trypsinization, washed twice with PBS (Sigma-Aldrich), lysed with a RIPA-buffer/Protease inhibitor cocktail (Sigma-Aldrich) by shaking on ice for 30 minutes followed by sonication on ice, 20 minutes centrifugation at 16′000×g, 4° C. Lysates were mixed 1:1 with 4× Lammli Buffer (Bio-Rad) prior to loading onto a Mini-PROTEAN TGX precast 4-20% PAGE gel (Bio-Rad). Gels were run for 1 hour at 110V. Gel were transferred to a Trans-Blot Turbo Mini PVDF membrane (Bio-Rad) using a Bio-Rad Trans-Blot Turbo System according to manufacturer's instructions. Equal amounts of cells were used, and the total transferred protein visualized on a Bio-Rad ChemiDoc Touch Imager prior to incubation with the primary antibody. After blocking for 1 hr with 5% non-fat-dry-milk (for p53 detection) or 5% BSA (for detection of p53-acetyllysine-382) in 1×TBS with 0.1% Tween-20 (Bio-Rad), blots were incubated overnight at 4° C. with p53 or p53-acetyl-lysine-382—specific antibodies (mouse monoclonal antibodies (SC-47698 from Santa Cruz Biotechnologies, Dallas, TX, USA), respectively rabbit polyclonal antibodies (#2525S from Cell Signaling Technologies, Boston, MA, USA), both at 1:1′000 dilution) and subsequently with the corresponding secondary HRP-conjugated antibodies (Cell Signaling Technologies, both 1:1′000 dilution) according to manufacturer's instructions. Specific antibody binding was detected with the SuperSignal West Femto Maximum Sensitivity Substrate (ThermoFisher) and visualized using a Bio-Rad ChemiDoc Touch Imager. Results from the experiment are shown in FIG. 25.

### EQUIVALENTS

Those skilled in the art will recognize, or be able to ascertain using no more than routine experimentation, many equivalents to the specific embodiments and methods described herein. Such equivalents are intended to be encompassed by the scope of the present application.

All patents, patent applications, and literature references cited herein are hereby expressly incorporated by reference.

1. (canceled)

2. A method according to General Scheme 1 or General Scheme 2 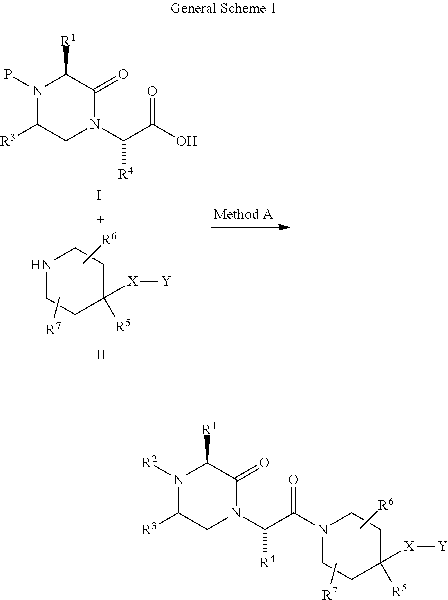 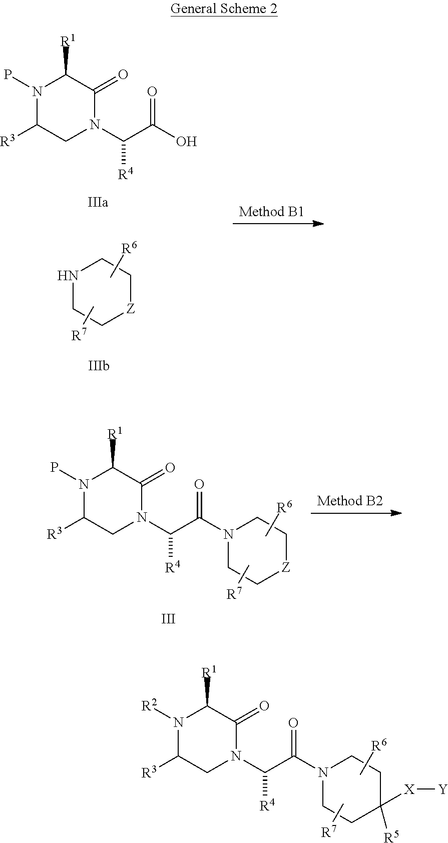
for producing a compound of formula (Ia) 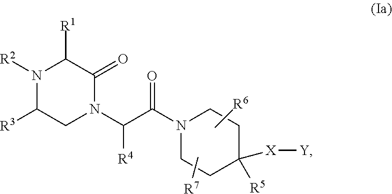
or a pharmaceutically acceptable salt, hydrate, solvate or stereoisomer thereof, wherein:
R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by cycloalkyl, aryl or heteroaryl, wherein the cycloalkyl, aryl or the heteroaryl is optionally substituted by halogen, C₁₋₄ alkyl or C₃₋₅ cycloalkyl;
R² is selected from H, C(O)R¹⁴, C(O)NR¹⁵R¹⁵, C(O)OR¹⁵, C₁₋₇ alkyl, C₂₋₇ alkenyl, C₂₋₇ alkynyl, C₃₋₇ cycloalkyl, C₄₋₇ cycloalkenyl, C₁₋₅ alkyl-OR^{B}, C₁₋₃ alkanediyl-O—C₁₋₃ alkanediyl-O—C₁₋₃ alkanediyl, C₁₋₅ alkyl-NHCOR¹³, or C₁₋₃ alkyl substituted by cycloalkyl, aryl or heteroaryl, wherein the cycloalkyl, aryl or the heteroaryl is optionally substituted by halogen, C₁₋₄ alkyl or C₃₋₅ cycloalkyl; with the proviso that when R² is C(O)NR¹⁵R¹⁵, both R can form a ring wherein the ring contains the N of NR¹⁵R¹⁵ and optionally one further heteroatom selected from O and N, wherein if the one further heteroatom is N, it is optionally substituted by R⁸;
R³ and R⁷ are each independently selected from H, C₁₋₇ alkyl, C₂₋₇ alkenyl, C₂₋₇ alkynyl, C₃₋₇ cycloalkyl, or C₄₋₇ cycloalkenyl, all optionally substituted by halogen, OR⁸, NR⁸R¹¹, or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by cycloalkyl, aryl or heteroaryl, wherein the cycloalkyl, aryl or the heteroaryl is optionally substituted by halogen, C₁₋₄ alkyl or C₃₋₅ cycloalkyl;
R⁵ is selected from H, C₁₋₇ alkyl, C₂₋₇ alkenyl, C₂₋₇ alkynyl, C₃₋₇ cycloalkyl, C₄₋₇ cycloalkenyl, OR⁸, C₁₋₃ alkyl-OR^{B}, or SR⁸; and wherein R⁵ can form a ring with any part of X or Y, wherein the ring optionally contains a carbonyl group;
R⁶ is selected from H, C₁₋₇ alkyl, C₂₋₇ alkenyl, C₂₋₇ alkynyl, C₃₋₇ cycloalkyl, C₄₋₇ cycloakenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; and wherein R⁶ can form a ring with any part of X; or is imidazolidinone;
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl;
X is selected from a bond, C₁₋₇ alkanediyl, C₂₋₇ alkenediyl, C₂₋₇ alkynediyl, C₃₋₉ cycloalkanediyl, C₄₋₆ cycloalkenediyl, —O—, C₁₋₃ alkanediyl-O—, —O—C₁₋₇ alkanediyl, —O—C₃₋₉ cycloalkanediyl, C₁₋₃ alkanediyl-O—C₁₋₇ alkanediyl, C₁₋₇ heteroalkanediyl, or —S—C₁₋₇ alkanediyl; and wherein X can form a ring or a polycyclic system with any part of R⁵, R⁶, or Y, wherein the ring optionally contains a carbonyl group;
Y is selected from H, C(O)NR¹⁰R¹², C(O)OR¹⁰, R¹⁰NC(O)NR¹⁰R¹², OC(O)R¹⁰, OC(O)NR¹⁰R¹², S(O)ₙR⁸ wherein n is 0, 1 or 2, SO₂NR¹⁰R¹², NR¹⁰SO₂R¹⁰, NR¹⁰R¹², HNCOR^{B}, CN, C₃₋₇-cycloalkyl optionally containing a heteroatom in the ring selected from O and N wherein if the heteroatom is N it is optionally substituted by R⁸; S-aryl, O-aryl, S-heteroaryl, O-heteroaryl wherein the S-aryl, O-aryl, S-heteroaryl, O-heteroaryl are optionally substituted by one or more R⁹ or R¹⁴; or aryl, heteroaryl wherein the aryl or heteroaryl is optionally substituted by one or more of R⁸; and wherein Y can form a ring with any part of X or R⁵, wherein the ring optionally contains a carbonyl group; with the proviso that when Y is C(O)NR¹⁰R¹² or NR¹⁰R¹², R¹⁰ and R¹² can form a ring wherein the ring contains the N of NR¹⁰R¹² and optionally one further heteroatom selected from O and N, wherein if the one further heteroatom is N, it is optionally substituted by R⁸;
R⁹ is selected from H, halogen, C₁₋₅ alkyl, C₂₋₅ alkenyl, C₂₋₅ alkynyl, C₃₋₅ cycloalkyl, C₁₋₅ alkyl-OR^{B}, C₁₋₅ alkyl-SR^{B}, C₁₋₅ alkyl-NR⁸R¹¹, C₁₋₅ alkyl-C(O)OR^{B}, C₁₋₅ alkyl-C(O)NR⁸R¹¹, C₁₋₅ alkyl-C(O)R¹⁰, CN, C(O)R⁸, C(O)NR⁸R¹¹, C(O)OR⁸, NR⁸C(O)NR⁸R¹¹, OC(O)NR⁸R¹¹, SO₂NR⁸R¹¹, NR⁸SO₂R⁸, OR⁸, NR⁸R¹¹, or S(O)ₙR⁸ wherein n is 0, 1 or 2;
R¹⁰ and R¹² are each independently selected from H, C₁₋₇ alkyl, C₂₋₇ alkenyl, C₂₋₇ alkynyl, C₃₋₇ cycloalkyl, C₄₋₇ cycloalkenyl, C₁₋₃ alkanediyl-O—C₁₋₃ alkanediyl-O—C₁₋₃ alkanediyl, C₁₋₃ alkyl-aryl, or C₁₋₃ alkyl-heteroaryl, all these groups optionally substituted by halogen, OR⁸, or NR⁸R¹¹;
R¹³ is C₁₋₅ alkyl substituted by a bicyclic ring optionally containing at least one heteroatom and a carbonyl group;
R¹⁴ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by aryl or heteroaryl, wherein the aryl or the heteroaryl is optionally substituted by halogen, C₁₋₄ alkyl or C₃₋₅ cycloalkyl;
each R¹⁵ is independently selected from H, C₁₋₇ alkyl, C₂₋₇ alkenyl, C₂₋₇ alkynyl, C₃₋₇ cycloalkyl, C₄₋₇ cycloalkenyl, OR⁸, or C₁₋₃ alkyl-OR^{B}; and
P is a suitable protecting group such as t-Boc or nosyl.

3. The method according to claim 2, wherein Method A in General Scheme 1 involves the use of a dehydrating agent in a suitable solvent, and optionally wherein the dehydrating agent is DCC (dicyclohexylcarbodiimide) or HATU (Hexafluorophosphate Azabenzotriazole Tetramethyl Uronium) and/or wherein the suitable solvent is DMF (dimethylformamide) or NMP (N-Methyl-2-pyrrolidone).

4. The method according to claim 2, wherein Method B1 in General Scheme 2 involves the use of a dehydrating agent in a suitable solvent, and optionally wherein the dehydrating agent is DCC (dicyclohexylcarbodiimide) or HATU (Hexafluorophosphate Azabenzotriazole Tetramethyl Uronium) and/or wherein the suitable solvent is DMF (dimethylformamide) or NMP (N-Methyl-2-pyrrolidone).

5. The method according to claim 2, wherein the compound of formula (Ia) is produced according to General Scheme 1 and the Precursor II used and the corresponding compound produced are as shown in Table 2.

6. The method according to claim 5, where in the compound of formula (Ia) is 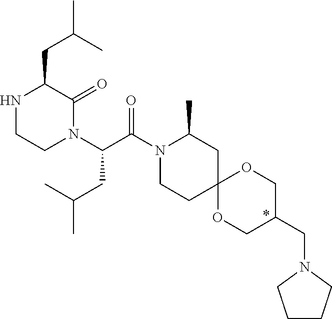 [SMILES: CC(C)C[C@@H]1NCCN([C@@H](CC(C)C)C(=O)N2CCC3(C[C@@H]2C)OCC(CN2CCCC2)CO3)C1=O]
and the Precursor II is

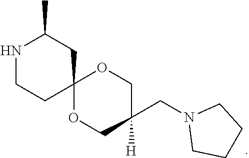 [SMILES: C[C@H]1C[C@]2(CCN1)OC[C@H](CN1CCCC1)CO2]

7. The method according to claim 2, wherein the compound of formula (Ia) is produced according to General Scheme 2 and the synthesis of Precursor III and the corresponding compound produced are as shown in Table 3.

8. The method according to claim 2, wherein the Precursor I is prepared according to General Scheme 3:

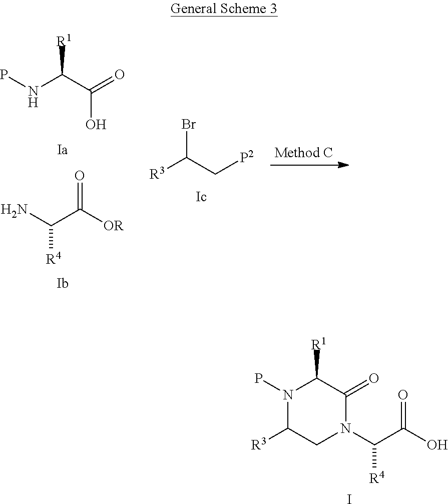

9. The method according to claim 8, wherein the compound of formula (Ia) is produced according to General Scheme 1, and the Precursors Ia, Ib, Ic for producing Precursor I and the corresponding produced are as shown in Table 1.

10. A method of producing a Precursor III according to Method B1 below, 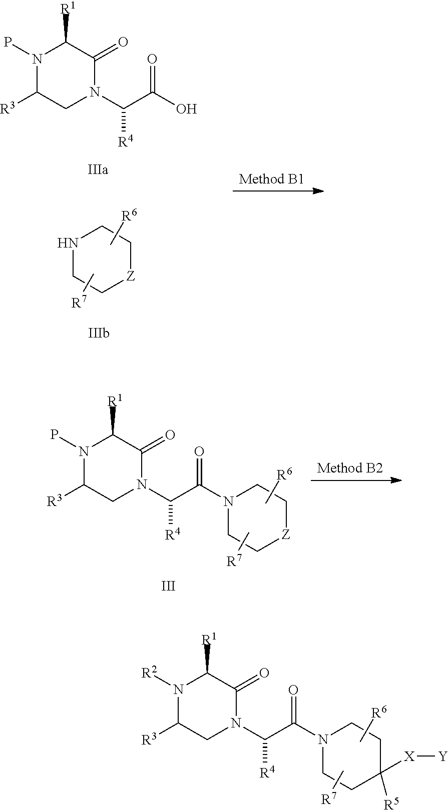
wherein R¹ is selected from H, C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by cycloalkyl, aryl or heteroaryl, wherein the cycloalkyl, aryl or the heteroaryl is optionally substituted by halogen, C₁₋₄ alkyl or C₃₋₅ cycloalkyl;
R³ and R⁷ are each independently selected from H, C₁₋₇ alkyl, C₂₋₇ alkenyl, C₂₋₇ alkynyl, C₃₋₇ cycloalkyl, or C₄₋₇ cycloalkenyl, all optionally substituted by halogen, OR^{B}, NR⁸R¹¹, or C₁₋₃ alkyl substituted by aryl or heteroaryl, wherein the aryl or the heteroaryl is optionally substituted by halogen, C₁₋₄ alkyl or C₃₋₅ cycloalkyl;
R⁴ is selected from C₁₋₇ alkyl, C₂₋₇ alkenyl, C₂₋₇ alkynyl, C₃₋₇ cycloalkyl, C₄₋₇ cycloalkenyl, or C₁₋₃ alkyl substituted by cycloalkyl, aryl or heteroaryl, wherein the cycloalkyl, aryl or the heteroaryl is optionally substituted by halogen, C₁₋₄ alkyl or C₃₋₅ cycloalkyl;
R⁶ is selected from H, C₁₋₇ alkyl, C₂₋₇ alkenyl, C₂₋₇ alkynyl, C₃₋₇ cycloalkyl, C₄₋₇ cycloakenyl, all optionally substituted by halogen, OR⁸, NR⁸R¹¹; C₁₋₃ alkyl substituted by C(O)NR⁸R¹¹; or C₁₋₃ alkyl substituted by aryl or heteroaryl, wherein the aryl or the heteroaryl is optionally substituted by halogen, C₁₋₄ alkyl or C₃₋₅ cycloalkyl; and wherein R⁶ can form a ring with any part of X; or is imidazolidinone; and
R⁸ and R¹¹ are each independently selected from H, C₁₋₇ alkyl, C₂₋₇ alkenyl, C₂₋₇ alkynyl, C₃₋₇ cycloalkyl, or C₄₋₇ cycloalkenyl.

11. The method of producing a Precursor III according to claim 10, wherein Z is selected from the group consisting of: CHCH₂C(O)OCH₃, CHCH₂C(O)OCH₂CH₃, CHCH₂CH₂NHBOC, CHOH, CHCH₂OH, CHCH₂(1H-imidazole), CHCH₂CH₂OH, CHC(O)OCH₃, and C═O.
